(19) United States
(12) Patent Application Publication
(10) Pub. No.: US 2026/0253670 A1
(43) Pub. Date: Aug. 27, 2026

(54) SYSTEMS AND METHODS FOR DIAGNOSING A DISEASE OR A CONDITION

(71) Applicant: Icahn School of Medicine at Mount Sinai, New York, NY (US)

(72) Inventors: Stuart Sealfon, New York, NY (US); Xi Chen, New York, NY (US); Zijun Zhang, New York, NY (US); Olga G. Troyanskaya, Princeton, NJ (US); Zidong Zhang, Princeton, NJ (US); Weiguang Mao, Pittsburgh, PA (US); Maria Chikina, Pittsburgh, PA (US); Daniel Chawla, New Haven, CT (US); Steven Kleinstein, New Haven, CT (US)

(21) Appl. No.: 19/108,141

(22) PCT Filed: Sep. 1, 2023
(86) PCT No.: PCT/US2023/073358

§ 371 Date: Feb. 28, 2025

Related U.S. Application Data
(60) Provisional application No. 63/403,687, filed on Sep. 2, 2022.

(51) Int. Cl. G16B 20/30; C12Q 1/6883; G16B 30/10; G16B 40/20; G16B 40/30

(52) U.S. Cl. CPC G16B 20/30; C12Q 1/6883; G16B 30/10; G16B 40/20; G16B 40/30; C12Q 2600/158

(57) ABSTRACT

Systems and methods for diagnosing a disease, a condition, or a characteristic in a subject are provided. In one such method, a future severity of an infection or inflammatory disease in a subject afflicted with the infection or inflammatory disease is predicted by obtaining a plurality of methylation levels. Each respective methylation level in the plurality of methylation levels represents a corresponding methylation level at a CpG site at a corresponding genetic locus in a plurality of genetic loci in a biological sample obtained from the subject. The plurality of methylation levels are inputted into a model comprising a plurality of parameters, where the model applies the plurality of parameters to the plurality of methylation levels to generate as output from the model an indication as to future severity of an infection or inflammatory disease in the subject.

### CROSS REFERENCE TO RELATED APPLICATIONS

This Application claims priority to U.S. Provisional Patent Application Ser. No. 63/403,687, entitled “Systems and Methods for Diagnosing a Disease or a Condition,” filed Sep. 2, 2022, which is hereby incorporated by reference in its entirety for all purposes.

### STATEMENT REGARDING FEDERALLY SPONSORED RESEARCH OR DEVELOPMENT

This invention was made with government support under N6600119C4022, awarded by the Defense Advanced Research Projects Agency (DARPA), by 9700130 awarded by Defense Health Agency through the Naval Medical Research Center, by R01 GM071966 awarded by the National Institute of Health (NIH), and by DK046943 awarded by the National Institute of Health (NIH). The government has certain rights in the invention.

### TECHNICAL FIELD

This specification describes using various computational tools to diagnose a disease or a condition.

### BACKGROUND

Standard tests for diagnosing a disease, a condition or an infection involve a variety of technologies including PCR assays, and antigen-binding assays, microbial cultures to name a few.

Despite the diversity and progress in technologies, standard tests generally share common design principle, which is to a detect a mutation, a defective protein, enzyme, or quantify the presence of a pathogen in patient samples. However standard tests have poor detection, false positive or negative results.

To overcome these limitations, there is a need in the art for new systems and methods for diagnosing accurately and effectively various characteristics, conditions and/or diseases and/or infections.

### SUMMARY

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

Advantageously, the present disclosure provides robust techniques for identifying a disease, or a condition in a subject.

One aspect of the present disclosure provides a method for determining a SARS-CoV-2 infection status of a test subject. The method includes sequencing a plurality of mRNA molecules from a biological sample obtained from the test subject, which obtains a plurality of sequence reads of RNA from the test subject. The method further includes aligning each respective sequence read in the plurality of sequence reads to a reference human transcriptome, thereby obtaining a corresponding plurality of aligned sequence reads. Moreover, the method includes using the corresponding plurality of aligned sequence reads to determine a corresponding spliced in amount for each respective alternative splicing event in a plurality of alternative splicing events, in which each respective alternative splicing event in the plurality of alternative splicing events is for a corresponding gene in a plurality of genes. Furthermore, the method includes, responsive to inputting the corresponding spliced in amount for each alternative splicing event in the plurality of alternative splicing events into a model obtaining, as output from the model, a SARS-CoV-2 infection status of the test subject.

Another aspect of the present disclosure provides a method for constructing a model that determines whether a subject is afflicted with a condition. The method comprises: A) for each respective first subject in a first plurality of subjects not afflicted with the condition, obtaining a first RNA-seq dataset comprising a respective discrete attribute value for each gene transcript in a corresponding first plurality of gene transcripts, for each cell in a respective first plurality of cells from a corresponding first biological sample from the respective first subject, and obtaining a first ATAC-seq dataset comprising a respective ATAC fragment count for each corresponding ATAC peak in a corresponding first plurality of ATAC peaks, for each respective cell in a respective second plurality of cells from a corresponding second biological sample from the respective subject. The method further comprises B) for each respective second subject in a second plurality of subjects afflicted with the condition, obtaining a second RNA-seq dataset comprising a respective discrete attribute value for each gene transcript in a corresponding second plurality of gene transcripts, for each cell in a respective third plurality of cells from a corresponding third biological sample from the respective second subject, and obtaining a second ATAC-seq dataset comprising a respective ATAC fragment count for each ATAC peak in a corresponding second plurality of ATAC peaks, for each respective cell in a respective fourth plurality of cells from a corresponding fourth biological sample from the respective subject. The first RNA-seq dataset and the second RNA-seq dataset are used to identify a plurality of candidate genes having differential transcription. The first ATAC-seq dataset and the second ATAC-seq dataset are used identify a plurality of candidate ATAC peaks having differential accessibility between the first plurality of subjects and the second plurality of subjects. For each respective transcription factor motif in a plurality of transcription factor motifs, the respective transcription factor motif is mapped onto the plurality of candidate ATAC peaks form a plurality of mapped transcription factor motifs. A model is constructed that determines whether a subject is afflicted with a condition using ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

Another aspect of the present disclosure provides a method for predicting a protective immune response level to a subsequent SARS-CoV-2 infection in a subject is provided. The method comprises (a) measuring DNA methylation in a plurality of genomic regions using a biological sample taken from the subject before infection, (b) measuring DNA methylation in the plurality of genomic regions using a biological sample taken from the subject during infection, (c) comparing the pattern of DNA methylation in the plurality of genomic regions between (a) and (b); and (d) predicting the protective immune response level based on the comparison of the pattern of DNA methylation in step (c). In this aspect of the present disclosure, when the pattern of DNA methylation in the plurality of genomics regions is similar between (a) and (b), the immune response level to a subsequent SARS-CoV-2 infection in a subject is predicted to be non-protective.

Another aspect of the present disclosure provides a method of evaluating a gene signature associated with a target condition that can afflict a host species is provided, where the gene signature comprises a first plurality of positive genes that are up-regulated when the test subject has the target condition and a second plurality of genes that are down-regulated when the test subject has the target condition. The method comprises A) obtaining an indication of each gene in the first plurality of positive genes; B) obtaining an indication of each gene in the second plurality of negative genes; C) obtaining a plurality of datasets, where each dataset in the plurality of datasets includes transcriptional data for each respective subject in a corresponding plurality of subjects and an indication of whether the respective subject has or does not have a respective test condition in a plurality of test conditions, the plurality of datasets includes at least one dataset for each test condition in the plurality of test conditions, and at least one test condition in the plurality of test conditions is the target condition. For each respective dataset in a plurality of datasets, for each respective time point in a set of time points represented by the respective dataset, for each respective subject in the respective dataset, a score is determined for the respective subject at the respective time point by determining a difference between a geometric mean of abundance values for the first plurality of positive genes and a geometric mean of abundance values for the second plurality of positive genes indicated in the respective dataset, and an area under a receiver operator characteristic curve (AUROC) value is determined for the respective dataset for the test condition using the respective score for each subject in the respective dataset at each respective timepoint. A performance of the gene signature is evaluated using the AUROC value of each dataset in the plurality of datasets associated with the target condition. Further, a cross-reactivity of the gene signature from the AUROC value of each dataset is evaluated in the plurality of datasets associated with a test condition that is other than the target condition.

Another aspect of the present disclosure provides a method for detecting a SARS-CoV-2 infection in a test subject. The method comprises measuring the transcriptional level of expression and/or measuring the epigenetic level of a set of signature genes in a blood sample from the test subject, where the set of signature genes comprises PIF1, BANF1, ROCK2, DOCK5, SLK, TVP23B, GUDC1, ARAP2, SLC25A46, TCEAL3, EHD3, and wherein the blood sample comprises plasmablast cells and T cells.

Another aspect of the present disclosure provides a method for determining whether a subject has a characteristic. The method comprises sequencing a plurality of mRNA molecules from a biological sample obtained from the subject, thereby obtaining a plurality of sequence reads of RNA from the subject; aligning each respective sequence read in the plurality of sequence reads to a reference human transcriptome, thereby obtaining a corresponding plurality of aligned sequence reads; using the corresponding plurality of aligned sequence reads to determine a corresponding transcript abundance in a plurality of transcript abundances, wherein each respective transcript abundance in the plurality of transcript abundances represents a transcript abundance of a corresponding gene in a plurality of genes; and inputting the plurality of transcript abundances into each respective neural network in a plurality of neural networks. Each respective neural network in the plurality of neural networks represents a different gene set in a plurality of gene sets, and each respective neural network in the plurality of neural networks comprises: (a) a corresponding plurality of input nodes, each respective input node in the corresponding plurality of input nodes for a different transcript abundance in the plurality of transcript abundance abundances, and (b) a representation of the corresponding gene set in the form of (i) a corresponding plurality of hidden nodes, each hidden node representing a gene in the corresponding gene set, and (ii) a corresponding plurality of edges, where each edge in the corresponding plurality of edges interconnects an input node in the plurality of input nodes to a hidden node in the corresponding plurality of hidden nodes with a corresponding edge weight, responsive to the inputting, obtaining a plurality of predictions, each prediction in the plurality of predictions from a neural network in the plurality of neural networks; and responsive to inputting the plurality of predictions into an ensemble model obtaining, as output form the ensemble model a prediction of whether the subject has the characteristic.

Another aspect of the present disclosure provides a method for predicting gene regulation mechanisms. The method comprises: (a) measuring chromatin accessibility and gene expression from single cell multi-omics datasets; (b) selecting regulatory regions comprising one or more proximal transcription start site (TSS) regions and one or more distal TSS regions; and (c) identifying one or more transcription factors (TFs) involved in regulating one or more target genes.

Another aspect of the present disclosure provides a predictive machine learning model. In some embodiments, the data is reduced to latent variables (LVs) using PLIER which incorporates outside prior information, such as pathways. In some embodiments, specific set of informative LVs are selected. In some embodiments, a machine learning (ML) model is trained.

### INCORPORATION BY REFERENCE

All publications, patents, and patent applications mentioned in this specification are herein incorporated by reference in their entireties for all purposes to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an exemplary system topology including a computer system, in accordance with an exemplary embodiment of the present disclosure.

FIGS. 2, 3A, and 3B collectively illustrate an overview of MAGICAL for mapping disease-associated regulatory circuits from scRNA-seq and scATAC-seq data. FIG. 2 illustrates a chart depicting that, in the 3D genome, the altered gene expression in cells between disease and control conditions can be attributed to the chromatin accessibility changes of proximal and distal chromatin sites regulated by TFs. (b) To identify disease-associated regulatory circuits in a selected cell type (including ATAC assay cells and RNA assay cells from samples being compared), MAGICAL selects DAS as candidate regions and DEG as candidate genes. Then, the filtered ATAC data and RNA data of differentially accessible sites (DAS) and differentially expressed genes (DEG) are used as input to a hierarchical Bayesian framework pre-embedded with the prior TF motifs and TAD boundaries. The chromatin activity A is modelled as a linear combination of TF-peak binding confidence B and the hidden TF activity T, with contamination of data noise NA. The gene expression R is modelled as a linear combination of B, T, and peak-gene looping confidence L, with contamination of data noise NR. MAGICAL estimates the posterior probabilities P(B|A,T), P(T|A,B) and P(L|R,B,T) by iteratively sampling variables B, T, and L to optimize against the data noise NA and NR in both modalities. Finally, regulatory circuits with high posterior probabilities of B and L (e.g., a high confidence circuit with inferred interactions between TF1, Site2 and Gene1) are selected. The accuracy and cell type specificity of the inferred peak-gene looping interactions were evaluated by checking their enrichment with cell-type matched chromatin interactions in Hi-C experiments. For the identified TFs, peaks, and genes in circuits, the accuracy of each using independent ChIP-seq, scATAC-seq, and scRNA-seq data was checked. Finally, as a demonstration of the utility of MAGICAL, the circuit target genes were used as features to predict disease states.

FIGS. 4A, 4B, 4C, 4D, 4E, and 4F collectively illustrate validation of COVID-19-associated circuit chromatin sites and genes. FIG. 4A provides a chart depicting the systems and methods of the present disclosure applied to a COVID-19 PBMC single-cell multiomics dataset and identified circuits for the clinical mild and severe groups, respectively, in which the systems and methods validated the circuit-associated chromatin sites and genes using newly generated and independent COVID-19 single-cell datasets. FIG. 4B provides a chart depicting UMAPs of a newly generated independent scATAC-seq dataset including 16K cells from 6 COVID-19 subjects and 9K cells from 3 controls showed chromatin accessibility changes in CD8 TEM, CD14 Mono, and NK cell types. FIGS. 4C and 4D collectively depict the systems and methods of the present disclosure precision of MAGICAL selected circuit sites is significantly higher than the that of the original DAS, the nearest DAS to DEG or all DAS in the same TAD with DEG. FIGS. 4E and 4F collectively depict the precision of circuit genes are significantly higher than the that of DEG. FIGS. 4C and 4E collectively depict, for mild COVID-19, MAGICAL identified 645 sites in CD8 TEM, 599 sites in CD14 Mono and 148 sites in NK, regulating 153 genes, 183 genes and 60 genes, respectively. (d, f) For severe COVID-19, MAGICAL identified 78 sites, 202 sites and 62 sites in the three cell types, regulating 25 genes, 81 genes, and 26 genes, respectively. FIGS. 4C, 4D, 4E, and 4F collectively depict precision is defined as the proportion of the identified circuit sites/genes to be differentially accessible and differentially expressed in the same cell type between infection and control conditions in independent datasets. Results are presented as bar plots where the height represent the precision and the error bar represent the 95% confidence interval. Significance evaluation is done using two-side Fisher's exact test.

FIGS. 5A, 5B, 5C, 5D, 5E, 5F, 5G, 511, and 51 collectively illustrate MAGICAL accurately identified distal regulatory chromatin sites and epi-driven genes associated with S. aureus infection. FIG. 5A depicts collected PBMC samples from 10 MRSA infected, 11 MSSA-infected, and 23 healthy control subjects and generated same-sample scRNA-seq and scATAC-seq data using separate assays. FIG. 5B depicts UMAP of integrated scRNA-seq data with 18 PBMC cell subtypes. FIG. 5C depicts UMAP of integrated scATAC-seq data with 13 PBMC cell subtypes. Under-represented subtypes including cDC1, CD4, TEM, CD8 CTL, pDC, and Plasmablast, altogether representing less than 5% of cells in the scRNA-seq data, were not recovered from the scATAC-seq data. FIG. 5D depicts the number of MAGICAL-identified regulatory circuits for each cell type and in contrast analysis. FIG. 5E depicts the number of shared and specific circuits between cell types. FIG. 5F depicts enrichment of circuit peak-gene interactions in each cell type with cell type-specific pcHi-C interactions. FIGS. 5G, 511, and 51 collectively depict analyzed MAGICAL-identified regulatory circuits for CD14 monocytes. FIG. 5G depicts TF motif enrichment analysis in circuit sites showed that AP-1 proteins are mostly significantly enriched at chromatin regions with increased accessibility in the infection condition. The log 2FC is calculated for each TF by dividing the number of binding sites with increased chromatin activity in the infection condition by the number of sites with decreased activity. FIG. 5G depicts, in total, 633 circuit sites were identified by MAGICAL. In comparison to all accessible chromatin sites, an increased proportion of circuit sites were in the range of 15 Kb to 25 Kb relative to gene TSS. The center points represent the fold change between the proportion of circuit sites and background sites in each window. The upper and lower points represent the 95% confidence interval. FIG. 51 depicts the circuit genes were significantly enriched with experimentally confirmed epi-genes in monocytes. All significance evaluation is assessed using the adjusted p-value of one-side hypergeometric test.

FIGS. 6A and 6B collectively illustrate an overview of MAGICAL-identified circuit genes robustly predict S. aureus infection and bacteria antibody sensitivity. FIG. 6A depicts circuit genes in common to MRSA and MSSA infections achieved a near-perfect classification of S. aureus infected and uninfected samples in multiple independent datasets (one adult dataset and two pediatric datasets). FIG. 6B depicts circuit genes that differed between MRSA and MSSA showed predictive value of antibiotic sensitivity in independent patient samples (three pediatric datasets).

FIG. 7 illustrates an overview of distribution learning of the hidden TF activity. Within one cell type of a sample, the systems and methods of the present disclosure assume that the distribution of TF activity (regulatory effect of a protein), is identical across cells from the same sample, regardless of if those cells are sequenced by the ATAC assay or RNA assay. However, there are no protein level measures so the TF activity is a hidden variable and needs to be estimated. Although precisely estimating the TF activity in each cell can be hard, its distribution can be learned from the multiomcs data. MAGICAL iteratively learns the TF activity distribution, approximates TF activities in individual cells by drawing samples from the learned distribution, and fits chromatin accessibility and gene expression data respectively using the estimated TF activity and other already estimated variables to optimize against data noise in both modalities.

FIGS. 8A and 8B collectively illustrate an overview of benchmarking MAGICAL and existing methods on one condition single cell multiomics data. FIG. 8A depicts the precision of peak-gene interactions identified by each method using the 10×PBMC multiome dataset, with validation on experimental chromatin interactions in blood cells curated in the 4DGenome database. MAGICAL identified 3721 peak-gene interactions. FIG. 8A depicts the precision of peak-gene interactions identified by each method using the GM12878 SHARE-seq dataset, with validation on distal chromatin interactions captured by an H3K27ac HiChIP experiment in GM12878 cell line. MAGICAL identified 5177 peak-gene interactions. Two baseline approaches are included in the comparisons as references: (1) for each candidate gene, pairing all sites with it if in the same TAD; (2) for each gene, pairing the nearest peak with it based on their genomic distance. Results were presented as boxplots where the center line represented the median of the precision after n=50 rounds of random sampling and the error bar represented the 95% confidence interval of the precision. The significance p-value was assessed using two-wide Fisher's exact test.

FIGS. 9A, 9B, 9C 9D, and 9E collectively illustrate an overview of COVID-19 PBMC validation of scATAC-seq data integration and peak calling using quality cells. FIG. 9A depicts distribution of TSS enrichment and nucleosome ratio of cells in scATAC-seq data of 8 samples. FIG. 9B depicts the number of peaks called per cell type using MACS2. Peaks are annotated as distal (>2 Kb), proximal (<2 Kb), exonic or intronic. FIGS. 9C and 9D collectively depict UMAPs of cells in the integrated scATAC-seq data with number and color representing conditions (FIG. 9C) or samples (FIG. 9D). FIG. 9E shows PBMC scATACseq quality cell QC information.

FIGS. 10A and 10B collectively illustrate an overview of S. aureus PBMC scRNA-seq data integration using quality cells. FIG. 10A depicts distribution of number of features (transcript) in quality cells selected for each disease sample. FIG. 10B depicts percent of mitochondrial of quality cells selected for each disease sample. FIGS. 10C and 10D collectively depict UMAPs of cells in the integrated object with color representing conditions or samples. Cells from all samples were well mixed in individual cell clusters, with rand index 0.016.

FIGS. 11A, 11B, 11C and 11D collectively illustrate an overview of S. aureus PBMC scATAC-seq data integration and peak calling using quality cells. (a) Distribution of TSS enrichment and nucleosome ratio of selected quality cells for each sample. (b) The number of peaks called per cell type using MACS2. Peaks are annotated as distal (>2 Kb), proximal (<2 Kb), exonic or intronic. FIGS. 11C and 11D depict UMAPs of cells in the integrated scATAC-seq data with number and color representing conditions (c) or samples (d). Cells from all samples were well mixed in individual cell clusters, with rand index 0.033.

FIGS. 12A, 12B, 12C, 12D, 12E, and 12F collectively illustrate an overview of integrated scRNA-seq and scATAC-seq data for MRSA, MSSA, and uninfected control samples. FIGS. 12A and 12B depicts UMAP of scRNA-seq data for each sample group with color representing cell types. FIGS. 12C and 12D depicts UMAP of scATAC-seq data for each sample group with number and color representing cell types. FIG. 12E depicts UMAPs of gene expression of cell type markers in the identified cell types. FIG. 12F depicts UMAPs of chromatin accessibility (gene TSS+body) of cell type markers.

FIG. 13 illustrates an overview of number of DEG or DAS identified for each contrast analysis within individual cell types.

FIG. 14 illustrates an overview of number of validating the inferred TF-chromatin region linkage in MAGICAL circuits in CD14 monocytes using ChIP-seq data from the Cistrome database. MAGICAL identified AP-1 proteins as top regulators in the circuits. During the assessment of chromatin region similarity between circuit chromatin sites and top 1000 peaks in each ChIP-seq profile (human) in the Cistrome database, JUN and FOS are top ranked too.

FIG. 15 illustrates an overview of number of enrichment of inflammatory disease GWAS loci in circuit chromatin sites. Results are presented as enrichment z-score for MAGICAL-selected circuit chromatin sites in each cell type with inflammatory diseases GWAS loci (including celiac disease, Crohn's disease, inflammatory bowel disease, type 1 diabetes, multiple sclerosis, primary biliary cirrhosis, rheumatoid arthritis, systemic lupus erythematosus, ulcerative colitis, psoriasis), or with GWAS loci of control diseases (Alzheimer's, ADHD, bipolar depression, Schizophrenia, Parkinson's, type 2 diabetes). Dots represent individual diseases (n=10 for inflammatory diseases and n=6 for control diseases). Central values represent the median z-score, the box extends from the 25th to the 75^{th} percentile, and the whiskers extend to the maximum and minimum values no further than 1.5 times the interquartile range from the hinge. With each cell type, GWAS traits with fewer than 5 overlapped loci with circuit sites were hold out from this evaluation. The significance p-value between enrichment scores of two disease groups was assessed using two-wide Wilcoxon ranksum test.

FIGS. 16A, 16B, 16C, 16D, 16E, and 16F collectively illustrate an overview of validating circuit genes on independent microarray datasets. FIG. 16A depicts S. aureus versus control prediction AUCs for models that are trained with circuit genes selected above each individual cutoff (n=20 rounds of running). FIG. 16B depicts S. aureus vs control differential expression π-values of 117 circuit genes identified using the systems an methods of the present disclosure and 366 standard DEG in the validation microarray datasets. Significance p-value is assessed using one-side Wilconxin Ranksum test. FIG. 16C depicts MRSA vs MSSA prediction AUCs for models that were trained with circuit genes selected above each individual cutoff (n=20 rounds of running). FIG. 16D depicts MRSA versus MSSA prediction AUCs for models that were trained with DEG selected above the same cutoff (n=20 rounds of running). Central lines in boxplots represent the median value, the box extends from the 25th to the 75th percentile, and the whiskers extend to the maximum and minimum values no further than 1.5 times the interquartile range from the hinge. FIG. 16E depicts ROC curves of predictive DEG selected by a Minimum Redundancy Maximum Relevance (MRMR) algorithm. FIG. 16F depicts ROC curves of predictive DEG selected by LASSO regression.

FIGS. 17A and 17B illustrates a schematic of the SARS-CoV-2 study design and alignment of subjects by infection timing. FIG. 17A Examples of three subject trajectories are shown arranged by study time (top) and infection pseudo-time, aligned by diagnosis (bottom). FIG. 17B Participants and samples are summarized by gender, race, ethnicity, and reported symptoms. All analyses of methylation changes associated with SARS-CoV-2 infection used preinfection samples as the Control group. The methylation data from the 28 never infected participants were used for the model evaluation of this group, n.a., not applicable; NA, not available.

FIGS. 18A, 18B, 18C, 18D, 18E and 18F collectively illustrate prolonged blood DNA methylation changes in asymptomatic and mild SARS-CoV-2 infections. FIG. 18A illustrates a number of DMS or DEG in each pseudotime period vs. pre-infection controls (nominal p<10-4). Numbers were either corrected for cell type proportions or uncorrected. FIG. 18B illustrates scatter plots of differential methylation at the sites in FIG. 18A for asymptomatic (n=68) versus mild (n=65) infections. FIGS. 18C, 18D and 18E illustrate scatter plots of differential expression (log 2 fold change) or methylation (normalized delta-beta) at the indicated periods for the DEG and DMS in FIG. 1D of Mao et al., 2023, “A methylation clock model of mild SARS-CoV-2 infection provides insight into immune dysregulation,” Molecular Systems Biology 19 e:11361 which is hereby incorporated by reference. FIG. 18F illustrates scatter plots comparing the changes in methylation levels compared with control following asymptomatic (n=68) and mildly symptomatic (n=65) infections for the First and Mid time period. These plots correspond to the same analysis shown for EarlyPost and LatePost in FIG. 18B.

FIGS. 19A, 19B, and 19C collectively illustrate characteristics of differential methylation following SARS-CoV-2 infection. FIG. 19A Schematic showing the features evaluated by enrichment analysis for association with postinfection hypomethylated sites in each DMS cluster. FIG. 19B illustrates enrichment of TFBS by cluster within a 200-bp window centered at each DMS. FIG. 19C illustrates Top five pathways showing enrichment of DMS-associated genes in each cluster. In FIGS. 19B and 19C, FDR<0.05 for at least one cluster, fold, fold enrichment.

FIGS. 20A, 20B, 20C, 20D, 20E, 20F, and 20G collectively illustrate a SARS-CoV-2 infection methylation clock. FIG. 20A illustrates regression model predicting time since infection at a top portion, and correlation and significance of models restricted to shorter time windows at a bottom portion. FIG. 20B illustrates comparison of the ten most frequently utilized sites when regression models are repeatedly generated for each time window. FIG. 20C illustrates accuracy of binary blood methylation classification models as the AUC, in distinguishing samples from pre-infection, infection, and post-infection pseudotime periods. FIG. 20D illustrates accuracy of blood methylation multiclass classifier in classifying samples from time periods relative to infection. FIG. 20E illustrates a schematic of the procedure utilized for nested cross-validation of all machine learning models generated. The left panel indicates one outer iteration for developing the model M built from the training set. The right side gives the data summary derived from all outer iterations. FIGS. 20F and 20G. Comparison of multiclass classifier performance on samples from male and female participants. 20F, Receiver operator curve obtained from multiclass classifier applied to samples from female participants. The 95% confidence intervals are indicated in the key. 20G, Receiver operator curve obtained from multiclass classifier applied to samples from male participants. The 95% confidence intervals are indicated in the key.

FIGS. 21A, 21B, 21C, 21D, 21E, and 21F illustrate Post-SARS-CoV-2 infection methylation pattern comparison with other conditions. FIG. 21A illustrates performance of a binary classifier trained to distinguish postinfection (EarlyPost or LatePost) vs. controls in other datasets. * marks current study datasets. “SARSCoV-2 Sero− vs. Sero+”: retrospective study dataset of Marine recruits exposed during late March-early April 2020, assayed for blood DNA methylation in mid-July, and distinguished by SARS-CoV-2 serology status. “Arrival at Quarantine vs. Later”: PCR-negative study participants upon arrival vs. later during training. FIG. 21B illustrates Receiver operator curve and significance of AUC for datasets showing FDR<0.05 in panel (A). FIGS. C and D illustrate enrichment of 20 most significantly hypomethylated DMS ranked by absolute delta beta values relative to top hypomethylated DMS in EarlyPost (C) or LatePost (D) vs. Control. FIG. 21E illustrates top-ranked hypomethylated DMS upon SARS-CoV-2 infection compared with other diseases showing enrichment in (C, D). Sites identified both in the SARS-CoV-2 study and at least one other condition are highlighted. Light gray sites were ranked in this study but not assayed in other studies. Gene annotations are indicated. FIG. 21F summarizes the datasets from infections and inflammatory diseases used in the present study. Abbreviations: NA, not available; n.a., not applicable.

FIGS. 22A, 22B, 22C, 22D, and 22E illustrate how persistent methylation state predicts future infection trajectories. FIG. 22A is a schematic illustration of the trained immunity phenomenon and expectations of possible protective and antiprotective effects of the post-SARS-CoV-2 methylation state. FIG. 22B illustrates a correlation between maximum relative viral level during infection and the probabilities of misclassification as EarlyPost (Left) using a multiclassifier model; correlation of two hypomethylated IFI44L sites with viral load (Right). A.U., arbitrary units, calculated as 80-(minimum cycle threshold PCR result) for each participant. FIG. 22C illustrates postinfection-like state is significantly associated with negative outcomes following SARS-CoV-2 infection in an older cohort with severe outcomes. As infection outcomes and postinfection probabilities (see FIG. 22E) are both associated with age, age was regressed out from the input methylation data for this analysis, showing these results are independent of subject age. The boxplot displays the 25th, 50th, and 75th percentiles, with whiskers that extend up to 1.5 times the interquartile range or the range of the data, whichever is smaller. P-values are from the Wilcoxon rank-sum test. FIG. 22D illustrates how there is no significant difference comparing samples following BCG vaccination of human subjects or BCG stimulation in vitro with respect to the model prediction probabilities as post-SARS-CoV-2 infection. The boxplot displays the 25th, 50th, and 75th percentiles, with whiskers that extend up to 1.5 times the interquartile range or the range of the data, whichever is smaller. P-values are from the Wilcoxon rank-sum test. FIG. 22E illustrates application of the multiclass classifier on a reference methylation cohort shows a strong positive correlation between age and prediction probabilities as Post. Results are comparable in males and females.

FIG. 23A illustrates a processing pipeline used for RNA-Seq data normalization in accordance with some embodiments of the present disclosure.

FIG. 23B illustrates processing pipeline used for methylation data normalization, in accordance with some embodiments of the present disclosure.

FIGS. 24A, 24B, 24C, 24D, and 24E collectively illustrate a multi-objective framework to identify a COVID-19 transcriptional signature. FIG. 24A illustrates a data compendium was curated to support the two main goals of the optimization framework, COVID-19 detection and cross-reactivity. The detection component included COVID-19 blood transcriptomes, ATAC-seq data and pathway knowledgebase; the cross-reactivity component included blood transcriptomes on viral, bacterial and non-infectious conditions.

FIG. 24B illustrates an optimization framework was based on a multi-objective fitness function that evaluated any proposed signature along three dimensions: detection, consistency with ATAC-seq and pathways, and cross-reactivity. An ideal (‘utopia’) signature would have high detection in COVID-19 studies, high consistency with ATAC-seq and pathways, and no detection in non-COVID-19 studies. FIG. 24C illustrates a fitness function was optimized in training studies with a genetic algorithm that returned a population of high-fitness solutions. To avoid over-fitting to the training studies, candidate signatures were then evaluated in independent development studies. Signature selection was based on proximity to the utopia point in both training and development studies. FIG. 24D illustrates a detection and cross-reactivity of the selected signature was tested against a third set of validation studies. FIG. 24E illustrates a framework included a strategy based on deconvolution of bulk transcriptomes and single cell data analysis, to infer the cell types that contribute to the signature performance.

FIGS. 25A, 25B, 25C, and 25D collectively illustrate identification of an 11-gene COVID-19 transcriptional signature. FIG. 25A illustrates a scatter plot, in which each point in the scatter plot corresponds to a candidate solution returned by the optimization framework. The selected signature (black point) satisfied the following criteria: (i) consistently low distance from the ideal signature when evaluated on training and development studies; (ii) high signature stability. The signature stability measured how often the genes in a signature appear also in other signatures. A higher stability favored a more robust selection process. FIG. 25B illustrates distributions of AUROC values were obtained by evaluating the signature on all the studies used for signature selection, both training and development. The color code corresponds to the four main study classes: COVID-19, other viral, bacterial, and non-infectious contrasts. The point size represents the study sample size. FIG. 25C illustrates a network shows functional, blood-specific connections involving the signature genes, and their pathway annotation as obtained from Greene et al., 2015. FIG. 25D illustrates genes in the selected signature showed high consistency between their RNA-seq scores and ATAC-seq scores. Scores were defined by combining the significance p-value and the fold-change for each gene in a single metric.

FIGS. 26A, 26B, 26C, and 26D collectively illustrate multi-cohort validation of the COVID-19 signature. FIG. 26A illustrates the COVID-19 signature was validated in multiple independent studies involving COVID-19 and non-COVID-19 contrasts. The study GSE1613151 provided data on three types of contrasts: COVID-19, viral respiratory infections, and bacterial respiratory infections. The ROC curves show the signature performance for these contrasts. FIG. 26B illustrates validation of the COVID-19 signature using the study GSE149689, providing data on COVID-19 and viral contrasts. FIG. 26C illustrates distributions of AUROC values in the four main study classes (COVID-19, other viral, bacterial, and non-infectious) were obtained by evaluating the signature on further independent validation studies from the public domain. FIG. 26D illustrates the COVID-19 signature performance was compared with that of four previously published signatures (σ1: Thair et al., 2021a; σ2: Lee et al., 2020; σ3: McClain et al., 2021; σ4: Aschenbrenner et al., 2021). For each signature and study class, the median AUROC values were obtained in the same set of validation studies. Furthermore, the significance of the resulting robustness and cross-reactivity were assessed based on hypothesis testing. Solid squares correspond to performance with p<0.05 based on a one-tailed t-test. Of the five signatures, only the signature optimized with this approach achieved significant performance for all study classes.

FIGS. 27A, 27B, and 27C collectively illustrate COVID-19 signature performance increases with disease severity. Three studies that included COVID-19 samples were used to explore whether the COVID-19 signature performance depended on severity. The three studies differed in the granularity of their annotations of COVID-19 disease severity. To harmonize the severity groups for analysis, the present disclosure defined three gradations: mild/moderate, severe, and critical. In some studies, mild/moderate also included asymptomatic cases, while critical also included cases that eventually resulted in death. FIG. 27A illustrates a study by Schulte-Schrepping et al. included (n=25) samples from mild and severe COVID-19 patients from the same cohort (Schulte-Schrepping et al., 2020). Shown are the distributions of the COVID-19 signature scores in the two groups (left panel), and the ROC curve showing signature performance when discriminating the mild and severe cases (right panel). The COVID-19 signature score in any given sample is defined as the geometric mean of expression levels of the up-regulated genes, minus the geometric mean of the expression levels of the down-regulated genes in the COVID-19 signature (see Methods). FIGS. 27B and 27C collectively illustrate three AUROC values correspond to the COVID-19 signature performance when discriminating each severity class from healthy samples in the study by the COMBAT consortium (FIG. 27B, n=99, COvid-19 Multi-omics Blood ATlas (COMBAT) Consortium, 2022) and the study by Stephenson et al. (FIG. 27C, n=113, Stephenson et al., 2021).

FIGS. 28A, 28B, and 28C collectively illustrate cell type changes explain COVID-19 signature performance. FIG. 28B illustrates a three-step strategy was developed to infer the immune cell types contributing to the identified COVID-19 signature. First, cell type specific signatures were retrieved from the Immune Response in Silico database (Abbas et al., 2005); second, each cell type signature was associated with a performance vector, a set of AUROC values produced by the signature in all the available studies; third, a combinatorial fit was applied to identify the combination of cell types whose performance vector best correlated with the performance vector associated with the COVID-19 signature. FIG. 28B illustrates a performance vector resulting from the combination of plasmablasts and memory T cells provided the best alignment with the COVID-19 performance vector. In the scatter plot, each point is a study, and its coordinates are the AUROC values for that study produced by the signature combining plasmablasts and memory T cells (x-axis), and by the COVID-19 signature (y-axis). FIG. 28C illustrates four subpanels show the AUROC distributions corresponding to the following four signatures: the COVID-19 signature, the plasmablasts' signature, the memory T cells' signature, and the signature combining plasmablasts and memory T cells. Solid (empty) boxplots indicate that the goals of detection and lack of cross-reactivity have (not) been satisfied based on hypothesis testing (p<0.05 based on a one-tailed t-test).

FIGS. 29A, 29B, and 29C collectively illustrate PIF1+EHD3+ plasmablasts as main mediators of COVID-19 detection. FIG. 29A illustrates a model of the COVID-19 signature performance, that connects the signature genes to plasmablasts and memory T cells according to their known specific expression in these cell types. These cell types play complementary roles for the signature: plasmablasts mediate COVID-19 detection, and memory T cells control against viral cross-reactivity. FIG. 29B illustrates a hypothesis that plasmablasts are major mediators of COVID-19 detection was tested in a single-cell RNA-seq study comparing COVID-19 against healthy controls. In a leave-one-out analysis for each cell type, removing plasmablasts (red point) produced the largest drop in COVID-19 detection. FIG. 29C illustrates in a leave-one-gene-out restricted to plasmablasts, removing PIF1 and EHD3 produced the largest drop in COVID-19 detection.

FIGS. 30A, 30B, 30C, 30D, and 30E collectively illustrate a curated set of human transcriptional infection signatures. FIG. 30A illustrates a standardized process was used to identify and curate published blood-based (whole blood or PBMC) transcriptional signatures of infection in humans from NCBI PubMed. Selection focused on signatures to detect general responses to viral (V) and bacterial (B) infections compared to control subjects. Signatures developed to differentiate viral from bacterial infections in a direct contrast (V/B) were also included. Signatures were parsed into positive (up-regulated with respect to the intended contrast) and negative (down-regulated) gene lists. Each signature was annotated with metadata including method of derivation, cohort details, and accessions for discovery datasets. Overall, this workflow produced 24 signatures curated for evaluation. FIGS. 30B, 30C, and 30D collectively illustrates a composition of each group of signatures (11 viral, 7 bacterial, and 6 V/B signatures) was characterized, including signature size, most frequently occurring genes and significantly enriched pathways (FDR<0.05, selected examples are displayed). Frequency of occurrence for each gene is listed in parentheses. Enrichments were computed based on the total pool of genes in each signature group. FIG. 30E illustrates pairwise Jaccard similarity coefficients were computed between signatures using concatenated positive and negative gene lists.

FIGS. 31A, 31B, 31C, 31D, 31E, and 31F collectively illustrate a compendium of human transcriptional infection datasets. FIG. 31A illustrates a standardized procedure was used to build a compendium of human transcriptional infection datasets profiling PBMCs or whole blood. After a systematic search of NCBI GEO, 150 datasets were selected that profile in-vivo responses to viral, bacterial, and parasitic infections, as well as immunomodulating non-infectious conditions. Datasets were passed through a standardized pre-processing pipeline. A total of 17,501 individual samples were annotated with condition type (e.g., infectious, non-infectious, healthy control) as well as infection type (e.g., viral, bacterial, parasitic) and the corresponding causative pathogen (e.g., influenza virus). Datasets were annotated with a study design (either cross-sectional or longitudinal). FIG. 31B illustrates datasets were labeled hierarchically by condition(s) profiled: infectious/non-infectious, viral/bacterial/other, and by unique pathogen. Within each layer of the hierarchy, bar heights correspond to the relative frequency of dataset labels. FIGS. 31C, 31D, and 31E collectively illustrates evaluated technical characteristics of the viral and bacterial datasets within this compendium that may impact downstream analyses. ‘The present disclosure compared the number of subjects per dataset (FIG. 31C), the number of datasets following each study design (FIG. 31D), the frequency of platform manufacturers (FIG. 31E), and the frequency of whole blood and PBMC samples (FIG. 31F).

FIGS. 32A, 32B, and 3C collectively illustrate establishing a general framework for signature evaluation. FIG. 32A illustrates, given a signature as input, a standardized evaluation framework was developed to calculate performance metrics across the data compendium. Signatures are scored for each subject in a target transcriptomic dataset using a geometric mean score approach that accommodates both cross-sectional and longitudinal study designs. The subject scores, paired with group labels, are used to compute an AUROC. AUROC statistics measuring performance for the intended and unintended conditions of a signature are reported as robustness and cross-reactivity, respectively. FIG. 32B illustrates a performance of curated signatures was computed in their respective discovery datasets. FIG. 32 illustrates how all 24 signatures were evaluated using geometric mean scoring and logistic regression scoring (see Methods). Performance was summarized for each signature as the median AUROC across evaluated datasets containing at least 15 cases and 15 controls. FIGS. 33A, 33B, 33C, 33D, 33E, 33F, 33G, 33H, 33I, 33J, and 33K collectively illustrate existing signatures of bacterial and viral infection are generally robust when evaluated in independent data. FIGS. 33A and 33B collectively illustrate viral (FIG. 33A) and bacterial (FIG. 33B) signature robustness was evaluated in independent datasets profiling intended infections and healthy controls. Ridge plots indicate AUROC distributions for each signature. Signatures with a median AUROC greater than 0.70 were considered robust. $ indicates a signature derived using non-infectious illness controls. FIG. 33C illustrates V/B signature robustness was evaluated by computing AUROCs for distinguishing viral infections from bacterial infections in independent datasets profiling both infection types. $ indicates a signature derived using non-infectious illness controls. FIGS. 33D and 33E collectively illustrate signature robustness was also evaluated separately for selected pathogens that were not included during signature discovery. Viral signature performance was evaluated in HIV infection (FIG. 33D), where the only available datasets were those profiling HIV infected subjects and healthy controls. Bacterial signature performance was evaluated in B. pseudomallei infection compared to healthy controls (FIG. 33E) and compared to non-infectious illness controls. FIG. 33F illustrates one dataset in the compendium (GSE103119, median V/B signature AUROC<0.50) was unique in its profiling of Mycoplasma infection. V/B signature AUROCs were compared for this dataset when including (+) or excluding (−) this pathogen (paired Wilcoxon signed-rank test). For FIGS. 33A, 33B, 33C, 33D, 33E, and 33F, distributions shown in color indicate signature robustness. FIG. 33G illustrates all 24 signatures were evaluated in male and female subjects separately. FIG. 33H illustrates a viral signature performance was compared between acute and chronic infection datasets (Wilcoxon signed-rank test). FIG. 331 illustrates a viral signature performance was compared between symptomatic and asymptomatic subjects in a dataset profiling H3N2 influenza virus infections. FIGS. 33J and 33K illustrate Viral (J) and bacterial (K) signature robustness was evaluated in independent datasets profiling intended infections and non-infectious controls. Ridge plots indicate AUROC distributions for each signature. $ indicates signatures derived using non-infectious controls.

FIGS. 34A, 34B, 34C, 34D, 34E, 34F, 34G, and 34H collectively illustrate nearly all infection signatures are cross-reactive with unintended infections or non-infectious conditions. FIG. 34A illustrates robust viral signatures were evaluated for cross-reactivity in datasets profiling bacterial infections and healthy controls. Signatures with median AUROCs greater than 0.60 were considered cross-reactive. FIG. 34B illustrates cross-reactivity was further separated by bacterial class, using datasets in the compendium where this information was available. C. Robust bacterial signatures were evaluated for cross-reactivity in datasets profiling viral infections and healthy controls. FIGS. 34D, 34E, and 34F collectively illustrate all 22 robust signatures were evaluated for cross-reactivity in parasitic infection (FIG. 34D), obesity (FIG. 34E), and aging (FIG. 34F) datasets. V/B signatures were considered cross-reactive if they had a median AUROC greater than 0.60 or less than 0.40 ($). This latter condition reflects that the designation of positive and negative genes in V/B signatures is arbitrary, and prediction in either direction is relevant to cross-reactivity. Signatures indicated in bold lettering were derived from discovery cohorts containing both pediatric and adult subjects. For FIGS. 34A, 34B, 34C, 34D, 34E, and 34F, distributions shown in color indicate a lack of signature cross-reactivity. FIGS. 34G and 34H illustrate how bacterial signature cross-reactivity was examined separately for different classes of viral pathogens, using datasets where this information was available. Viral classes were defined by presence of a viral envelope (FIG. 34G) and type of viral genome (FIG. 34H). Viral classes were included if at least 5 datasets profiled this type of pathogen. Distributions shown in color indicate a lack of signature cross-reactivity.

FIGS. 35A, 35B, 35C, 35D, 35E, 35F, 35G and 35H collectively illustrate analysis of influenza signatures demonstrates a trade-off between robustness and cross-reactivity. A targeted literature search for influenza signatures was performed as a case study of single-pathogen signatures. FIGS. 35B and 35C collectively illustrate robustness (FIG. 35B) and cross-reactivity (FIG. 35C) of influenza signatures were evaluated. General viral signature V10 was included as a positive control for viral detection. FIG. 35D illustrates a meta-analysis procedure used to develop V10, a signature that was not cross-reactive with unintended infections, was adapted to generate a pool of 124 candidate signature genes that discriminate influenza infection from healthy control samples. 100,000 synthetic signatures were generated by randomly sampling these candidate genes. Performance was characterized over the space of candidate signatures (gray shading depicting density). Signatures comprising the Pareto front (white points) were identified to define signatures with locally optimal robustness and cross-reactivity characteristics. Pink shading indicates proximity to an ideal influenza signature with perfect robustness and no cross-reactivity. FIG. 35E illustrates a similar analysis was carried out using a new set of candidate genes generated from the results of a meta-analysis directly contrasting influenza infection with non-influenza viral infection samples. FIG. 35F illustrates a local neighborhood along the Pareto front in (FIG. 35E) was defined (gray points), and the relationship between signature size and signature robustness was examined. FIG. 35G illustrates each synthetic signature was separated into two signatures by removing either its positive (black points) or negative (grey points) gene sets. Performance was evaluated independently for each of these signatures. FIG. 35H illustrates the correlation between cross-reactivity (<AUROC> in non-influenza studies) and signature size was examined for the Pareto front signatures (white points) and their local neighborhood (gray points). N=100 Pareto region signatures.

FIGS. 36A and 36B collectively illustrate exemplary methods for implementing an aspect of the present disclosure, in which optional embodiments are indicated by dashed boxes, in accordance with some embodiments of the present disclosure.

FIGS. 37A, 37B, and 37C collectively illustrate meta-analysis of COVID-19 mRNA training studies and correlation with ATAC-seq data. FIG. 37A illustrates a volcano plot shows the results of a meta-analysis of the COVID-19 contrasts. The aim of the meta-analysis was to identify a pool of genes differentially expressed across the COVID-19 contrasts used for signature training. The x-axis shows the combined effect size, while the y-axis shows the combined False Discovery Rate (FDR). Each point in the volcano plot is a gene. Red corresponds to up-regulated genes; blue to down-regulated genes; gray to genes not significantly regulated. FIG. 37B illustrates a scatter plot shows the relationship between RNA-seq data and ATAC-seq data. The x-axis and y-axis represent scores corresponding to RNA-seq and ATAC-seq data, respectively. For each gene, these scores aggregate the effect size and the statistical significance (see Methods). FIG. 37C illustrates a histogram shows the distribution of correlation values between RNA-seq scores and ATAC-seq scores for sets of genes randomly extracted from the pool of genes differentially expressed by COVID-19. The distribution provides a background reference to assess the significance of the correlation between RNA-seq scores and ATAC-seq scores corresponding to the selected COVID-19 signature.

FIGS. 38A, 38B, 38C, and 38D collectively illustrate an overview of stability analysis of the solution space. FIG. 38A illustrates a representation of a generic signature as a binary vector. Each component of the vector corresponds to a gene, and takes on the value of 1 or 0 depending on whether the gene belongs or does not belong to the signature. FIG. 38B illustrates, given a set of candidate signatures, the present disclosure introduced a stability metric at the gene and signature levels. The stability of a gene in the solution space is the frequency at which the gene appears across the solutions. After calculating the stability of each gene, the present disclosure computes the stability of any given signature as the average stability of its member genes. FIG. 38C illustrates a histogram shows the distribution of stability values across the solution space. The stability of the selected signature, indicated by the dashed vertical line, is larger than the mean of the distribution. FIG. 38D illustrates the stability value of genes in the selected signature (black segment), in the context of the background stability values of all genes (white histogram).

FIG. 39 illustrates an overview of a COVID-19 signature that is insensitive to age differences, in which boxplots show the distribution of COVID-19 signature scores for each sample (points) and for each study in the COVID-19 validation studies (facet) where information on age was available. The COVID-19 signature score in any given sample is defined as the geometric mean of expression levels of the up-regulated genes, minus the geometric mean of the expression levels of the down-regulated genes in the COVID-19 signature. The following three studies were considered: GSE149689 (n=17), GSE162562 (n=108), GSE166253 (n=23). The COVID-19 signature score in any given sample is defined as the geometric mean of expression levels of the up-regulated genes, minus the geometric mean of the expression levels of the down-regulated genes in the COVID-19 signature (see Methods). The p-values resulting from an ANOVA test to compare the signature scores across age groups were not significant (p>0.05).

FIG. 40 illustrates an overview of a COVID-19 signature that is insensitive to sex differences, in which boxplots show the distribution of COVID-19 signature scores for each sample (points) and for each study in the COVID-19 validation studies (facet) where information on sex was available. The COVID-19 signature score in any given sample is defined as the geometric mean of expression levels of the up-regulated genes, minus the geometric mean of the expression levels of the down-regulated genes in the COVID-19 signature. The following five studies were considered: GSE149689 (n=17), GSE152418 (n=34), GSE152641 (n=86), GSE162562 (n=108), GSE166253 (n=23). The p-values resulting from a t-test to compare the signature scores across sex groups were not significant (p>0.05).

FIG. 41 illustrates COVID-19 signature does not cross-react with pregnancy. A. The boxplot shows the distribution of COVID-19 signature scores (see Methods) for samples in study GSE108497. Each point is a sample from pregnant and non-pregnant healthy women (left panel, n=187). The ROC curve shows signature performance when discriminating pregnant and non-pregnant samples (right panel). B-C. COVID-19 signature scores and ROC curves when subsetting the data by pregnancy stage. The AUROC values were all lower than 0.5, indicating no signature cross-reactivity with pregnancy.

FIG. 42 illustrates an overview of AUROC distributions produced by previously published signatures in validation studies, in which four boxplots show the distribution of AUROC values obtained with four previously published COVID-19 signatures, denoted as σ₁, σ₂, σ and σ₄. For each signature and study class (COVID-19, viral, bacterial, and non-infectious), the present disclosure reports the AUROC values obtained in the same set of validation studies (n=43).

FIG. 43 provides an outline of a framework for interpretable machine learning that combines prior knowledge, bioinformatic analysis tools, and ensemble modeling in accordance with an aspect of the present disclosure.

FIG. 44 illustrates how an ensemble classifier in accordance with the present disclosure systematically improved the accuracy distribution observed with the individual neural networks.

FIG. 45 illustrates statistics on pre-processing of an annotation libraries in accordance with an embodiment of the present disclosure.

FIGS. 46A, 46B, 46C, 46D, and 46 illustrate application of the ensemble model of the present disclosure to kidney plant rejection.

FIGS. 48 and 49 illustrate normalization of Gene Set Enrichment Analysis (GSEA) scores to account for the diversity in library size and gene set size in accordance with an embodiment of the present disclosure.

FIGS. 50A, 50B, 50C, 50D, 50E, and 50F collectively illustrate global analysis of base learners for pathway and regulatory annotation libraries in accordance with an embodiment of the present disclosure.

FIGS. 52A, 52B and 52C collectively illustrate exemplary methods for determining whether a subject has a characteristic using a neural network ensemble method in which optional blocks are indicated by dashed boxes in accordance with an aspect of the present disclosure.

FIGS. 53A, 53B, and 53C collectively illustrate the motivation and workflow for identification of cis-regulatory circuitry in accordance with an embodiment of the present disclosure. FIG. 53A depicts percentage of eQTLs and enhancers from gold standard databases located inside and outside of ATAC peaks called in a human PBMC single nucleus multiome data. Reference blood eQTLs are obtained from the GTEx DAPG fine-mapped eQTLs database. Reference blood enhancers are obtained from the enhancerAtlas database. FIGS. 53B and 53C depict a schematic of a method in accordance with the present disclosure in which single nucleus multiome (RNAseq+ATACseq within each cell) is taken as input, and scanned for potential cis-TF binding sites by motif analysis. A linear model is fitted for gene expression as a function of chromatin accessibility and TF expression to each cell in the dataset to select highly significant regulatory circuits. The circuits identified are supported by the coincidence of TF expression, binding site accessibility and target gene expression within individual cells.

FIGS. 54A, 54B, 54C and 55D collectively illustrate an overview of performance and utility of the methods and systems of part 6 of the present disclosure. FIG. 54A depicts number of regulatory circuits identified by TRIPOD 12 and CREMA at false discovery rate cutoff=0.005. The circuits from CREMA were categorized as “inside called peaks” or “outside called peaks” depending on whether the binding site of the circuit overlapped with any chromatin peak. Because the circuit inference from TRIPOD was restricted to the chromatin peaks, all the circuits from TRIPOD are inside called peaks. FIG. 54B depicts percentage of true regulatory regions recovered by TRIPOD and CREMA when controlling for the precision in the peak regions. Predictions from the two methods were selected at different FDR cutoffs to calculate the precision of regulatory peak prediction and recovery of true, regulatory regions from the reference gold standards (see methods of part 6). Reference blood eQTLs are obtained from the GTEx DAPG fine-mapped eQTLs database. Reference blood enhancers are obtained from the enhancerAtlas database. FIGS. 54C and 54D depict cis-regulatory domains outside of called peaks resolve major cell types in human PBMC and mouse pituitary respectively. UMAP dimension reductions were calculated by using only the accessibilities of the cis-regulatory domains discovered outside of ATAC peaks as features. Cell type annotations were from independent analysis using the expression of known marker genes (see methods of part 6).

FIGS. 55A, 55B, 55C, 55D and 55E collectively illustrate an overview of Gata2-Pcsk1 circuit in the pituitary gonadotrope cells. FIG. 55A is a schematic showing the analysis of Gata2 circuits by CREMA in the mouse pituitary and validation by differentially expressed genes in the conditional Gata2 knockout data. (p=3.5×10⁻⁶, Z=4.5, df=1, one-sided z-test of two proportions). FIG. 55B depicts detailed view of an identified Gata2-Pcsk1 circuit where Gata2 interacts with a cis regulatory domain located ~61 kb upstream of the TSS of Pcsk1. Normalized accessibilities were plotted separately for cells with and without Pcsk1 expression. Zoomed in plot showing the detailed chromatin accessibility pattern around the Gata2 binding site (red arrow). FIG. 55C illustrates UMAPs showing the expression of Pcsk1 in the pituitary cells and the cell type annotations, and FIGS. 55D and 55E depicts Box plot and point plot showing the pseudobulk RNA of Pcsk1 and pseudobulk ATAC of the Gata2 site in each cell type of the wild type mouse pituitary samples (n=3) and gonadotrope conditional Gata2 knockout samples (n=3).

FIGS. 56A, 56B, and 56C collectively illustrate an overview of regulatory circuitry of human immune cells. FIG. 56A depicts selected identified TF modules and their activities in immune cell types in accordance with the present disclosure. FIG. 56B depicts selected identified regulatory circuits in the TCF7 module that are shared between naive T cells and central memory T cells, and circuits in the TCF7 module that are specific to one of the two cell types in accordance with the present disclosure. GO terms annotated to these target genes are labeled below. FIG. 56C depicts example of a queried gene LTA and the list of identified regulatory circuits targeting this gene in accordance with the present disclosure.

FIG. 57 illustrates an overview of percentage of eQTLs and enhancers from gold standard databases that locate inside and outside of ATAC peaks called in a human PBMC single nucleus multiome data in accordance with the present disclosure.

FIGS. 58A and 58B illustrate an overview of percentage of true regulatory regions recovered by TRIPOD and by the systems and methods of the present disclosure when controlling for the precision in the peak regions. Predictions from the two methods were selected at different FDR cutoffs to calculate the precision of regulatory peak prediction and recovery of true regulatory regions from the gold standards.

FIG. 59 illustrates an overview of expression of Gata2 in the mouse pituitary tissue (upper) and the corresponding cell type annotations in the same UMAP space (lower) in accordance with the present disclosure.

FIGS. 60A, 60B, 60C, 60D, 60E, 60F, 60G, 60H, 60I, 60J, 60K, 60L, 60M, 60N, 600, 60P, 60Q, 60R, 60S, 60T, 60U, 60V, 60W, 60X, and 60Y illustrate COVID-19 host regulatory circuits identified by MAGICAL in which COVID-19-associated circuit genes, chromatin sites and regulatory TFs in each cell type in accordance with an embodiment of the present disclosure.

FIGS. 61A and 61B illustrate S. aureus PBMC scRNA-seq quality cell QC information, QC thresholds and the number of quality cells in each scRNA-seq profile, in accordance with an embodiment of the present disclosure.

FIG. 62 illustrates S. aureus. aureus PBMC scATACseq quality cell QC information, in accordance with an embodiment of the present disclosure.

### DETAILED DESCRIPTION

The implementations described herein provide various technical solutions for determining the status of a disease, condition, or infection in a test subject.

Advantageously, the present disclosure further provides various systems and methods for diagnosing a disease or a condition.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” means within 1 or more than 1 standard deviation, per the practice in the art. In some embodiments, “about” means a range of 20%, ±10%, +5%, or +1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. All numerical values within the detailed description herein are modified by “about” the indicated value, and consider experimental error and variations that would be expected by a person having ordinary skill in the art. The term “about” can have the meaning as commonly understood by one of ordinary skill in the art. In some embodiments, the term “about” refers to ±10%. In some embodiments, the term “about” refers to ±5%.

As used herein, the term “subject,” “training subject,” or “test subject” refers to any living or non-living organism, including but not limited to a human (e.g., a male human, female human, fetus, pregnant female, child, or the like) and/or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. The terms “subject” and “patient” are used interchangeably herein and can refer to a human or non-human animal who is known to have, or potentially has, a medical condition or disorder, such as, e.g., kidney disease. In some embodiments, a subject is a “normal” or “control” subject, e.g., a subject that is not known to have a medical condition or disorder. In some embodiments, a subject is a male or female of any stage (e.g., a man, a woman, or a child).

A subject from whom an image and/or biopsy is obtained using any of the methods or systems described herein can be of any age and can be an adult, infant or child. In some cases, the subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old).

As used herein, the terms “control,” “healthy,” and “normal” describe a subject and/or an image from a subject that does not have a particular condition (e.g., kidney disease), has a baseline condition (e.g., prior to onset of the particular condition), or is otherwise healthy. In an example, a method as disclosed herein can be performed to diagnose a renal disease and/or a kidney graft failure in a subject having a renal disease using a trained model, where the model is trained using one or more training images obtained from the subject prior to the onset of the condition (e.g., at an earlier time point), or from a different, healthy subject. A control image can be obtained from a control subject, or from a database.

The term “normalize” as used herein means transforming a value or a set of values to a common frame of reference for comparison purposes. For example, when one or more pixel values corresponding to one or more pixels in a respective image are “normalized” to a predetermined statistic (e.g., a mean and/or standard deviation of one or more pixel values across one or more images), the pixel values of the respective pixels are compared to the respective statistic so that the amount by which the pixel values differ from the statistic can be determined.

As used interchangeably herein, the terms “classifier”, “model” and “machine learning model” refers to a machine learning model or algorithm. In some embodiments, such a model is a supervised machine learning model. Nonlimiting examples of supervised learning models include, but are not limited to, logistic regression models, neural networks, support vector machines, Naive Bayes algorithms, nearest neighbor models, random forest models, decision tree models, boosted trees models, multinomial logistic regression, linear models, linear regression, GradientBoosting, mixture models, hidden Markov models, Gaussian NB models, linear discriminant analysis, or any combinations thereof. In some embodiments, a machine learning model is a multinomial classifier. In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level classifier).

Neuralnetworks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xᵢ, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in performing the methods disclosed herein. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes classifiers suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. Nearest neighbor models can be memory-based and include no model to be fit. For nearest neighbors, given a query point xo (a first image), the k training points x(r), r, . . . , k (here the training images) closest in distance to xo are identified and then the point xo is classified using the k nearest neighbors. In some embodiments, the distance to these neighbors is a function of the values of a discriminating set. In some embodiments, Euclidean distance in feature space is used to determine distance as d₍ᵢ₎=∥x₍ᵢ₎−x_{(O)}∥. Typically, when the nearest neighbor algorithm is used, the value data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Randomforest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (e.g., a linear classifier) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in a training dataset. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

The term “classification” can refer to any number(s) or other characters(s) that are associated with a particular property of a sample. For example, a “+” symbol (or the word “positive”) can signify that a sample is classified as having a desired outcome or characteristic, whereas a “−” symbol (or the word “negative”) can signify that a sample is classified as having an undesired outcome or characteristic. In another example, the term “classification” refers to a respective outcome or characteristic (e.g., high risk, medium risk, low risk). In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff value refers to a value above which results are excluded. In some embodiments, a threshold value is a value above or below which a particular classification applies. Either of these terms can be used in either of these contexts.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

The terms “sequence reads” or “reads,” used interchangeably herein, refer to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that vary in size from tens to hundreds to thousands of base pairs. Illumina parallel sequencing can provide sequence reads vary to a lesser extent (e.g., where most sequence reads are of a length of about 200 bp or less). A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes (e.g., in hybridization arrays or capture probes) or amplification techniques, such as the polymerase chain reaction (PCR) or linear amplification using a single primer or isothermal amplification.

As disclosed herein, the terms “sequencing,” “sequence determination,” and the like refer generally to any and all biochemical processes that may be used to determine the order of biological macromolecules such as nucleic acids or proteins. For example, sequencing data can include all or a portion of the nucleotide bases in a nucleic acid molecule such as a DNA fragment.

Several aspects are described below with reference to example applications for illustration. Numerous specific details, relationships, and methods are set forth to provide a full understanding of the features described herein. The features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

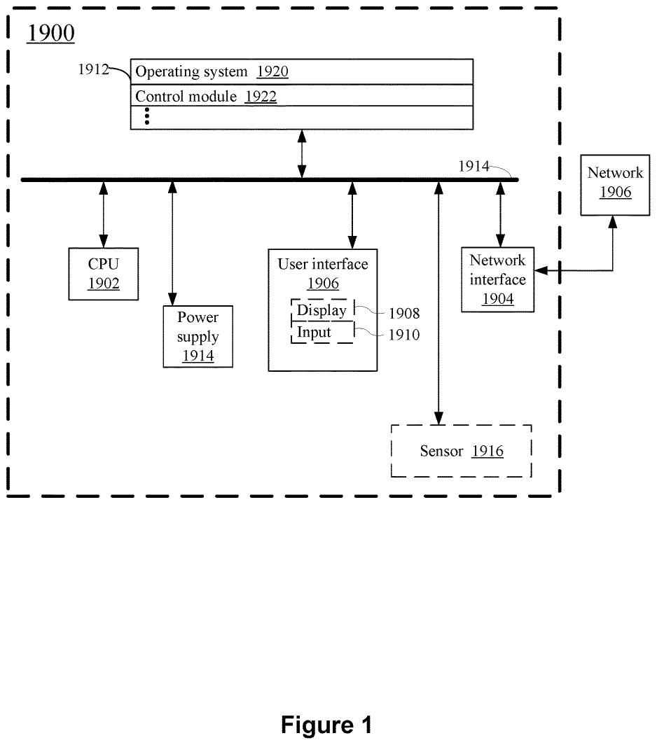

In the present disclosure, unless expressly stated otherwise, descriptions of devices and systems will include implementations of one or more computers. For instance, and for purposes of illustration in FIG. 1, a computer system 1900 is represented as single device that includes all the functionality of the computer system 1900. However, the present disclosure is not limited thereto. For instance, in some embodiments, the functionality of the computer system 1900 is spread across any number of networked computers and/or reside on each of several networked computers and/or by hosted on one or more virtual machines and/or containers at a remote location accessible across a communications network (e.g., communications network 1906 of FIG. 1). One of skill in the art will appreciate that a wide array of different computer topologies is possible for the computer system 1900, and other devices and systems of the preset disclosure, and that all such topologies are within the scope of the present disclosure. Moreover, rather than relying on a physical communications network 1906, the illustrated devices and systems may wirelessly transmit information between each other. As such, the exemplary topology shown in FIG. 1 merely serves to describe the features of an embodiment of the present disclosure in a manner that will be readily understood to one of skill in the art.

FIG. 1 depicts a block diagram of a distributed computer system (e.g., computer system 1900) according to some embodiments of the present disclosure. The computer system 1900 at least facilitates communicating one or more instructions for detecting epigenetic modifications of nucleic acids.

In some embodiments, the communication network 1906 optionally includes the Internet, one or more local area networks (LANs), one or more wide area networks (WANs), other types of networks, or a combination of such networks.

Examples of communication networks 1906 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.1 in), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 1900 includes one or more processing units (CPUs) 1902, a network or other communications interface 1904, and memory 1912.

In some embodiments, the computer system 1900 includes a user interface 1906. The user interface 1906 typically includes a display 1908 for presenting media. In some embodiments, the display 1908 is integrated within the computer systems (e.g., housed in the same chassis as the CPU 1902 and memory 1912). In some embodiments, the computer system 1900 includes one or more input device(s) 1910, which allow a subject to interact with the computer system 1900. In some embodiments, input devices 1910 include a keyboard, a mouse, and/or other input mechanisms. Alternatively, or in addition, in some embodiments, the display 1908 includes a touch-sensitive surface (e.g., where display 1908 is a touch-sensitive display or computer system 1900 includes a touch pad).

In some embodiments, the computer system 1900 presents media to a user through the display 1908. Examples of media presented by the display 1908 include one or more images (e.g., user interface on display 1908 presenting a chart of 3C, etc.), a video, audio (e.g., waveforms of an audio sample), or a combination thereof. In typical embodiments, the one or more images, the video, the audio, or the combination thereof is presented by the display 1908 through a client application. In some embodiments, the audio is presented through an external device (e.g., speakers, headphones, input/output (I/O) subsystem, etc.) that receives audio information from the computer system 1900 and presents audio data based on this audio information. In some embodiments, the user interface 1906 also includes an audio output device, such as speakers or an audio output for connecting with speakers, earphones, or headphones.

Memory 1912 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 1912 may optionally include one or more storage devices remotely located from the CPU(s) 1902. Memory 1912, or alternatively the non-volatile memory device(s) within memory 1912, includes a non-transitory computer readable storage medium. Access to memory 1912 by other components of the computer system 1900, such as the CPU(s) 1902, is, optionally, controlled by a controller. In some embodiments, memory 1912 can include mass storage that is remotely located with respect to the CPU(s) 1902. In other words, some data stored in memory 1912 may in fact be hosted on devices that are external to the computer system 1900, but that can be electronically accessed by the computer system 1900 over an Internet, intranet, or other form of network 106 or electronic cable using communication interface 1904.

In some embodiments, the memory 1912 of the computer system 1900 stores:
an operating system 1920 (e.g., ANDROID, iOS, DARWIN, RTXC, LINUX, UNIX, OS X, WINDOWS, or an embedded operating system such as VxWorks) that includes procedures for handling various basic system services;
an electronic address associated with the computer system 1900 that identifies the computer system 1900 (e.g., within the communication network 1906);
a control module 1922 including one or more modules 1924 for controlling one or more processes (e.g., method) associated with the computer system 1900; and
optionally, a client application for presenting information (e.g., media) using a display 1908 of the computer system 1900.

In some embodiments, the control module 1922 includes one or more models 1924 that is configured to perform one or more steps of a method of the present disclosure.

Part 1: Systems and Methods for Mapping Disease Regulatory Circuits at Cell-Type Resolution from Single-Cell Multiomics Data

In one aspect, the systems and methods of the present disclosure provide computational methods to identify chromatin differential accessible sites linked to differentially expressed gene using preferably scRNAseq and scATACseq data. The disclosed methods rely on linking potential regulatory sites and genes using TAD domains. The methods provide more robust identification of these features than other methods which facilitates their use as features for developing an accurate diagnostic test.

In some embodiments the systems and methods of the present disclosure assists in the development of diagnostic tests. In some embodiments the systems and methods of the present disclosure improves the feature selection step if the relevant data is available. Epigenetic signature to distinguish different subtypes of Staphylococcus Aureus (Staph) infections.

One aspect of the present disclosure provides a method for constructing a model that determines whether a subject is afflicted with a condition. The method comprises A) for each respective first subject in a first plurality of subjects not afflicted with the condition, obtaining a first RNA-seq dataset comprising a respective discrete attribute value for each gene transcript in a corresponding first plurality of gene transcripts, for each cell in a respective first plurality of cells from a corresponding first biological sample from the respective first subject and obtaining a first ATAC-seq dataset comprising a respective ATAC fragment count for each corresponding ATAC peak in a corresponding first plurality of ATAC peaks, for each respective cell in a respective second plurality of cells from a corresponding second biological sample from the respective subject. For each respective second subject in a second plurality of subjects afflicted with the condition, a second RNA-seq dataset is obtained comprising a respective discrete attribute value for each gene transcript in a corresponding second plurality of gene transcripts, for each cell in a respective third plurality of cells from a corresponding third biological sample from the respective second subject, and a second ATAC-seq dataset is obtained comprising a respective ATAC fragment count for each ATAC peak in a corresponding second plurality of ATAC peaks, for each respective cell in a respective fourth plurality of cells from a corresponding fourth biological sample from the respective subject.

The first RNA-seq dataset and the second RNA-seq dataset are to identify a plurality of candidate genes having differential transcription.

The first ATAC-seq dataset and the second ATAC-seq dataset are used to identify a plurality of candidate ATAC peaks having differential accessibility between the first plurality of subjects and the second plurality of subjects.

For each respective transcription factor motif in a plurality of transcription factor motifs, mapping the respective transcription factor motif onto the plurality of candidate ATAC peaks form a plurality of mapped transcription factor motifs.

A model is constructed that determines whether a subject is afflicted with a condition using ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

In some embodiments, each respective first plurality of cells comprises 50 cells, each respective second plurality of cells comprises 50 cells, each respective third plurality of cells comprises 50 cells, and each respective fourth plurality of cells comprises 50 cells.

In some embodiments, each corresponding first plurality of gene transcripts represents 50 or more genes, each corresponding first plurality of ATAC peaks comprises 50 or more peaks, each corresponding second plurality of gene transcripts represents 50 or more genes, each corresponding second plurality of ATAC peaks comprises 50 or more peaks.

In some embodiments, the plurality of candidate genes having differential transcription comprises 50 or more candidate genes, and the plurality of candidate ATAC peaks having differential accessibility comprises 50 or more candidate peaks.

In some embodiments, the first plurality of subjects comprises 25 or more subjects and the second plurality of subjects comprises 25 or more subjects.

In some embodiments, the first RNA-seq dataset is a single cell RNA-seq dataset, the second RNA-seq dataset is a single cell RNA-seq dataset, the first ATAC-seq dataset is a single cell ATAC-seq dataset, and the second ATAC-seq dataset is a single cell ATAC-seq dataset.

In some embodiments, the first RNA-seq dataset is a bulk RNA-seq dataset, the second RNA-seq dataset is a bulk RNA-seq dataset, the first ATAC-seq dataset is a bulk ATAC-seq dataset, and the second ATAC-seq dataset is a bulk ATAC-seq dataset.

In some embodiments, the first RNA-seq dataset, the second RNA-seq dataset, the first ATAC-seq dataset, and the second ATAC-seq dataset are determined using cells from the first and second plurality of subjects that have a common cell type. In some embodiments, the common cell type is T-cell or a CD14 cell. In some embodiments, the common cell type is B memory, B naïve, CD4 TCM, CD8 Naïve, CD8 TEM, CD14 Mono, CD16 Mono, cDC2, MAIT, NK, NK_CD56bright, Platelets, CD14 monocytes, CD16 monocytes, CD4 TCM cells, CD8 TEM cells, CD4 Naïve cells, or natural killer.

In some embodiments, a candidate gene in the plurality of candidate genes satisfied the proximity threshold with respect to a respective candidate ATAC peak when the candidate gene is within 20 kilobases, within 15 kilobases, within 10 kilobases, or within 5 kilobases of the respective candidate ATAC peak in a reference genome for the first and second plurality of subjects.

In some embodiments, the reference genome is a human reference genome.

In some embodiments, the condition is a pathogenic infection.

In some embodiments, the pathogenic infection is a Covid infection or a Staph infection.

In some embodiments, the pathogenic infection is a bacterial infection. In some embodiments the bacterial infection is a Streptococcal infection (e.g., Streptococcus pyogenes), Staphylococcal infection (e.g., methicillin-resistant Staphylococcus aureus), Salmonellosis, Tuberculosis, a urinary tract infection, Lyme Disease, Gonorrhea, Chlamydia, Diphtheria (Corynebacterium diphtheriae), or Pneumonia.

In some embodiments, pathogenic infection is a viral infection. In some embodiments the viral infection is influenza, COVID-19 (e.g., SARS-CoV-2), Chickenpox, Measles, Herpes Simplex, or HIV/AIDS.

In some embodiments, the condition is a disease.

In some embodiments, the model formation uses Bayesian analysis of ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

In some embodiments, the model comprises 1000, 10,000, 100,000 or 1×10⁶ parameters.

Another aspect of the present disclosure provides a computer system for constructing a model that determines whether a subject is afflicted with a condition. The computer system comprises one or more processors. The computer system further comprises memory addressable by the one or more processors. The memory stores at least one program for execution by the one or more processors, the at least one program comprising instructions for: A) for each respective first subject in a first plurality of subjects not afflicted with the condition, obtaining a first RNA-seq dataset comprising a respective discrete attribute value for each gene transcript in a corresponding first plurality of gene transcripts, for each cell in a respective first plurality of cells from a corresponding first biological sample from the respective first subject, and obtaining a first ATAC-seq dataset comprising a respective ATAC fragment count for each corresponding ATAC peak in a corresponding first plurality of ATAC peaks, for each respective cell in a respective second plurality of cells from a corresponding second biological sample from the respective subject. The at least one program further comprises instructions B) for each respective second subject in a second plurality of subjects afflicted with the condition, obtaining a second RNA-seq dataset comprising a respective discrete attribute value for each gene transcript in a corresponding second plurality of gene transcripts, for each cell in a respective third plurality of cells from a corresponding third biological sample from the respective second subject, and obtaining a second ATAC-seq dataset comprising a respective ATAC fragment count for each ATAC peak in a corresponding second plurality of ATAC peaks, for each respective cell in a respective fourth plurality of cells from a corresponding fourth biological sample from the respective subject. The at least one program further comprises instructions for C) using the first RNA-seq dataset and the second RNA-seq dataset to identify a plurality of candidate genes having differential transcription; and D) using the first ATAC-seq dataset and the second ATAC-seq dataset to identify a plurality of candidate ATAC peaks having differential accessibility between the first plurality of subjects and the second plurality of subjects. The at least one program further comprises instructions E) for each respective transcription factor motif in a plurality of transcription factor motifs, mapping the respective transcription factor motif onto the plurality of candidate ATAC peaks form a plurality of mapped transcription factor motifs; and F) constructing the model that determines whether a subject is afflicted with a condition using ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

In another aspect, provided herein is a non-transitory computer readable storage medium, wherein the non-transitory computer readable storage medium stores instructions, which when executed by a computer system, cause the computer system to perform and of the methods provided in the present disclosure.

Another aspect of the present disclosure provides a method for determining whether a subject is afflicted with an S. aureses infection in which a plurality of discrete attribute values is obtained. Each discrete attribute value in the plurality of discrete attribute values represents a transcript abundance of a respective gene in a plurality of genes in a biological sample from the subject, where the plurality of genes comprises three or more genes listed in Table 1.13. The plurality of discrete attribute values are inputted into a model comprising a plurality of parameters, where the model applies the plurality of parameters to the plurality of discrete attribute values to generate as output from the model an indication as to whether the subject is afflicted with the S. aureses infection.

In some embodiments, the plurality of genes comprises 4, 5, 6, 7, 8, 9, 10, 11, 12, 13, 14, 15, 16, 17, 18, 19, or 20 or more genes listed in Table 1.13. In some embodiments, the plurality of genes comprises 20, 30, 40, 50, 60, 70, 80, 90, 100, 110, or all 117 genes listed in Table 1.13. In some embodiments, the plurality of genes consists of 4, 5, 6, 7, 8, 9, 10, 11, 12, 13, 14, 15, 16, 17, 18, 19, or 20 more genes listed in Table 1.13. In some embodiments, the plurality of genes consists of between 10 and 20, between 10 and 30, between 20 and 40, between 20 and 50, between 30 and 60, between 30 and 70, between 40 and 80, between 40 and 90, between 50 and 100, between 50 110, or between 60 and 117 genes listed in Table 1.13.

In some embodiments, the plurality of discrete attribute values is obtained by bulk transcriptome sequencing of nucleic acids in the biological sample.

In some embodiments, the plurality of discrete attribute values is obtained by single cell transcriptome sequencing of nucleic acids in the biological sample.

In some embodiments a first gene in the plurality of genes is associated with the cell type CD14 Mono in Table 1.13. In some such embodiments, a second gene in the plurality of genes is associated with the cell type CD16 Mono in Table 1.13.

In some embodiments, the method further comprises obtaining, in electronic form, a plurality of sequence reads from the biological sample, where the plurality of sequence reads comprises at least 10,000 RNA sequence reads, and the plurality of sequence reads is used to determine each discrete attribute value in the plurality of discrete attribute values. In some embodiments this involves mapping each respective sequence read in the plurality of sequence reads to a reference genome.

In some embodiments, the biological sample is blood, whole blood, or plasma.

In some embodiments, the biological sample comprises a plurality of mRNA molecules and the obtaining the plurality of sequence reads further comprises sequencing the plurality of mRNA molecules using RNA sequencing.

In some embodiments, the plurality of sequence reads comprises at least 100,000, at least 1×10⁶, or at least 1×10⁷ sequence reads.

In some embodiments, the model is selected from the group consisting of: a logistic regression model, a neural network, a support vector machine, a Naive Bayes model, a nearest neighbor model, a boosted trees model, a random forest model, a decision tree, or a clustering model.

In some embodiments, the plurality of parameters comprises 100 or more parameters, 1000 or more parameters, 10,000 or more parameters, 100,000 or more parameters, or 1×10⁶ or more parameters.

In some embodiments, the indication as to whether the subject is afflicted with the S. aureses infection is a likelihood that the subject is afflicted with the S. aureses infection.

In some embodiments, the indication as to whether the subject is afflicted with the S. aureses infection is a binary indication as to whether or not the subject is afflicted with the S. aureses infection.

In some embodiments, the biological sample comprises serum, urine, cerebrospinal fluid, fecal, saliva, sweat, tears, pleural fluid, pericardial fluid, or peritoneal fluid from the subject.

In some embodiments, the biological sample consists of blood, whole blood, plasma, serum, urine, cerebrospinal fluid, fecal, saliva, sweat, tears, pleural fluid, pericardial fluid, or peritoneal fluid from the subject.

In some embodiments, the method further comprises treating the subject with a drug when the model indicates that the subject has an S. aureses infection. In some embodiments, the drug is cefazolin, nafcillin, oxacillin, vancomycin, daptomycin, linezolid, or a combination thereof.

### 1.1 Abstract

Resolving chromatin remodeling-linked gene expression changes at cell type resolution is important for understanding disease states. One aspect of the present disclosure provides an approach that leverages paired scRNA-seq and scATAC-seq data from different conditions to map disease-associated transcription factors, chromatin sites, and genes as regulatory circuits. By simultaneously modeling signal variation across cells and conditions in both omics data types, the present disclosure achieves high accuracy on circuit inference. The disclose approach is applied to study Staphylococcus aureus sepsis from peripheral blood mononuclear single-cell data generated from infected subjects with bloodstream infection and from uninfected controls. Sepsis-associated regulatory circuits were identified predominantly in CD14 monocytes, known to be activated by bacterial sepsis. The present disclosure addresses the challenging problem of distinguishing host regulatory circuit responses to methicillin-resistant (MRSA) and methicillin-susceptible Staphylococcus aureus (MSSA) infections. While differential expression analysis alone failed to show predictive value, the identified epigenetic circuit biomarkers of the present disclosure distinguished MRSA from MSSA.

### 1.2 Introduction

Gene expression can be modulated through the interplay of proximal and distal regulatory domains brought together in three-dimensional space. See Schoenfelder et al., 2019. Chromatin regulatory domains, transcription factors, and downstream target genes form regulatory circuits. See Kim et al., 2009. Within circuits, the binding of transcription factors to chromatin regions and the three-dimensional looping between these regions and gene promoters represent the mechanisms governing how transcription factors transform regulatory signals into changes in RNA transcription. See Drosophila et al., 2010; Marbach et al., 2016. In disease, these circuits could be dysregulated in a cell type specific manner and may not be observed from bulk samples. See Wilk et al., 2021. Therefore, identifying the impact of disease on regulatory circuits includes a framework for mapping regulatory domains with chromatin accessibility changes to altered gene expression in the context of cell-type resolution. See Krijger et al., 2016. Single-cell RNA sequencing (scRNA-seq) and single-cell assay for transposase-accessible chromatin using sequencing (scATAC-seq) characterizing disease states have improved the identification of differential chromatin sites and/or differentially expressed genes within individual cell types. See Wilk et al., 2021; Cao et al., 2018; Kreitmaier et al., 2022.

Yet, advances in single-cell assay technology have outpaced the development of methods to maximize the value of multiomics datasets for studying disease-associated regulation, especially for the regulatory interactions that are not directly measured by the omics data. Recent computational approaches to support the multiomics data analysis demonstrate the promise of this area but still lack the capacity to resolve regulation changes within individual cell types, which precludes elucidating regulatory circuits affected by the disease or showing different responses in varying disease states. See Stuart et al., 2019; Ma et al., 2020; Jiang et al., 2022; Cao et al., 2022, each of which is hereby incorporated by reference in its entirety for all purposes. To address these shortcomings, the present disclosure models coordinated chromatin accessibility and gene expression variation to identify circuits (both the units and their interactions) that differ between conditions. scRNA-seq and scATAC-seq data are concurrently analyzed using a hierarchical Bayesian framework. To accurately detect differences in regulatory circuit activity between conditions, hidden variables are used for explicitly modeling the transcriptomic and epigenetic signal variations between conditions and optimization against the noise in both scRNA-seq and scATAC-seq datasets. Because regulatory circuits are cell-type specific, see Javierre et al., 2016, which is hereby incorporated by reference in its entirety for all purposes, the present disclosure reconstructed them a cell-type resolution. The identified regulatory circuits were systematically benchmarked against multiple public datasets to support the accuracy of the circuits.

Staphylococcus aureus (S. aureus), a bacterium often resistant to common antibiotics, is a major cause of severe infection and mortality. See Arnold et al., 2006; Saavedra-Lozano et al., 2008, each of which is hereby incorporated by reference in its entirety for all purposes. Using single-cell multiomics data generated from peripheral blood mononuclear cell (PBMC) samples of S. aureus infected subjects and healthy controls, the present disclosure identified host response regulatory circuits that are modulated during S. aureus bloodstream infection, and circuits that discriminate the responses to methicillin-resistant (MRSA) and methicillin-susceptible S. aureus (MSSA). Genes in the host circuits accurately predicted S. aureus infection in multiple validation datasets. Moreover, in contrast to conventional differential analysis that failed to identify specific genes for robust antibiotic-sensitivity prediction, the present disclosure identified circuit genes can differentiate MRSA from MSSA. Therefore, the systems and methods of the present disclosure can be used for multiomics data-based gene signature development, providing a bioinformatic solution that can improve disease diagnosis.

### 1.3 Results

### 1.3.1 Framework

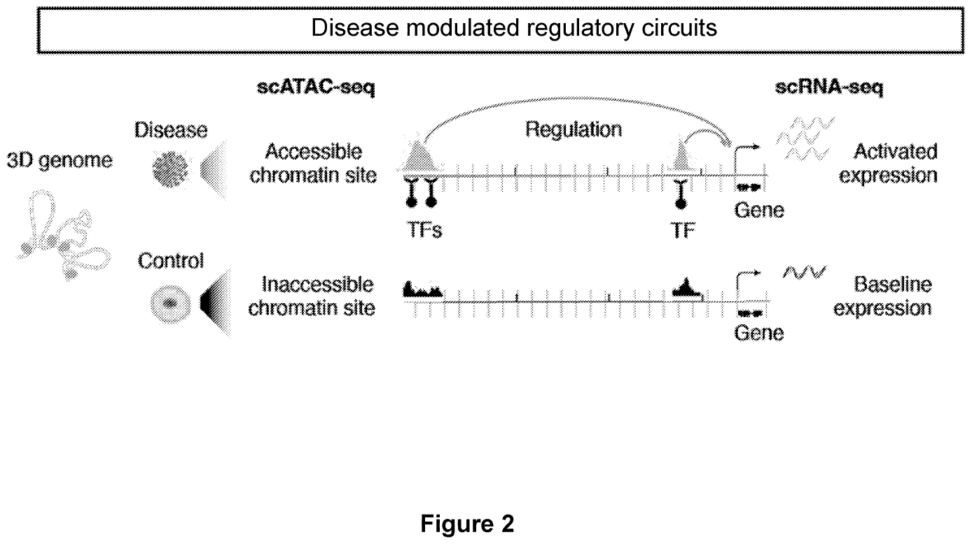

The present disclosure identifies disease-associated regulatory circuits by comparing single-cell multiomics data (scRNA-seq and scATAC-seq) from disease and control samples (FIG. 2).

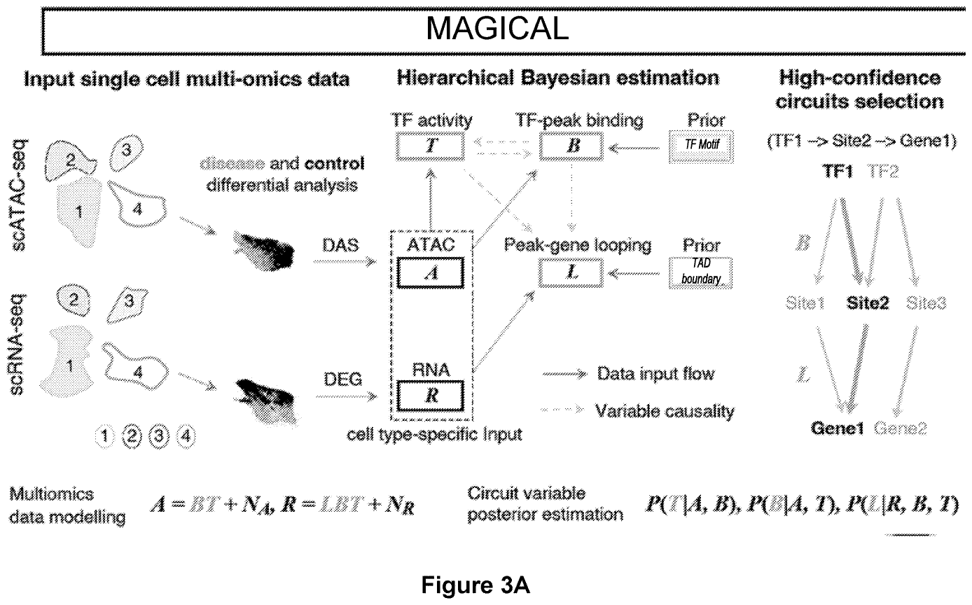

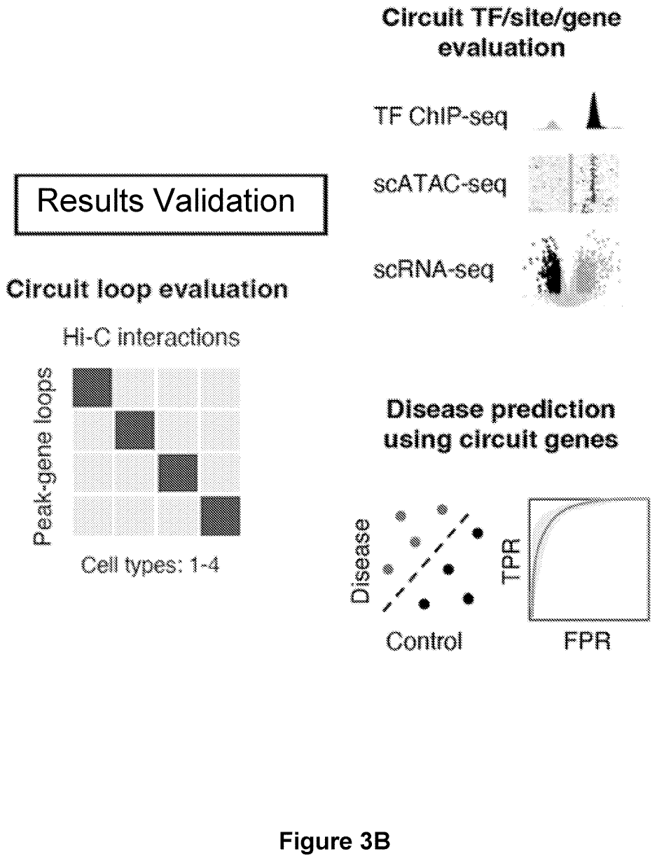

The present disclosure incorporates transcription factor (TF) motifs and, in some embodiments, chromatin topologically associated domain (TAD) boundaries, as prior information to infer regulatory circuits comprising chromatin regulatory sites, modulatory TFs, and downstream target genes for each cell type. In brief, to build candidate disease-modulated circuits, differentially accessible sites (DAS) within each cell type are first associated with TFs by motif sequence matching and then linked to differentially expressed genes (DEG) in that cell type by genomic localization within the same TAD. Next, model chromatin accessibility and gene expression variation are iteratively modeled across cells and samples in each cell type (e.g., using Bayesian analysis) to estimate the confidence of TF-peak and peak-gene linkages for each candidate circuit (FIG. 3A).

To accurately identify varying circuits between different conditions, signal and noise in chromatin accessibility and gene expression data is explicitly modeled. See Section 1.5.10, below. A TF-peak binding variable and a hidden TF activity variable are jointly estimated to fit to the chromatin accessibility variation across cells from the conditions being compared. These two variables are then used together with a peak-gene looping variable to fit the gene expression variation. Using Gibbs sampling, the present disclosure iteratively estimates variable values and optimizes the states of circuit TF-peak-gene linkages. Finally, high-confidence circuits fitting the signal variation in both data types are selected.

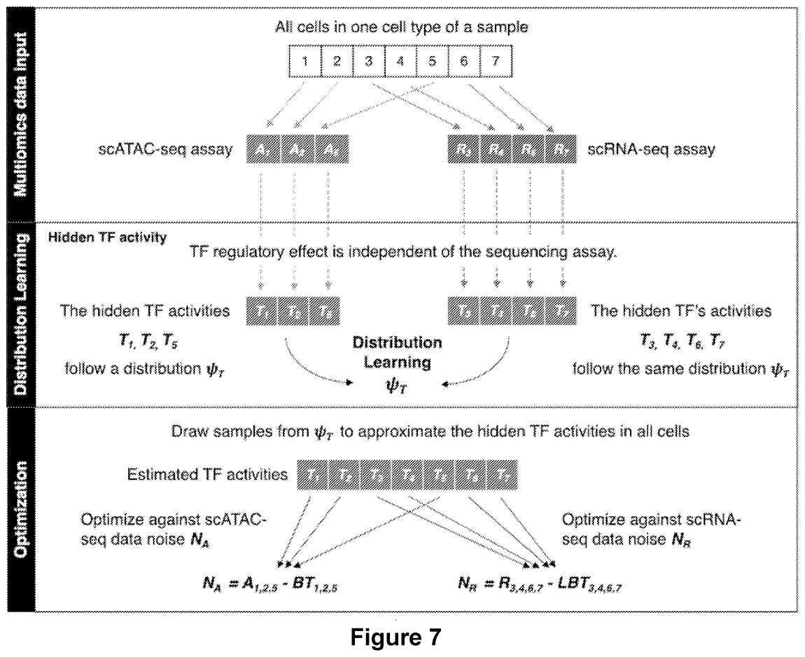

TF activity represents the regulatory capacity (protein level) of a particular TF protein, which is distinct from TF expression. See Liao et al., 2003; and Tran et al., 2005, each of which is hereby incorporated by reference in its entirety for all purposes. For each TF, the systems and methods of the present disclosure assume its hidden TF activities following an identical distribution across cells in the same cell type and the same sample, regardless of if the cells are from the scATAC-seq assay or the scRNA-seq assay or both. The systems and methods of the present disclosure iteratively learns the activity distribution for each TF and estimates the specific activities of all TFs in each cell (FIG. 7). This procedure eliminates the requirement of cell-level pairing of RNA-seq and ATAC-seq data. This procedure makes the systems and methods of the present disclosure a general tool that can analyze single-cell true multiome or sample-paired multiomics datasets.

The systems and methods of the present disclosure were validated in multiple ways, demonstrating that it infers regulatory circuits accurately (FIG. 3B). Linkages between chromatin sites and genes inferred using the systems and methods of the present disclosure were validated using experimental 3D chromatin interactions. The resulting circuit genes, peaks and their regulatory TFs were respectively evaluated in multiple independent studies. And finally, as one example of utility, the systems and methods of the present disclosure showed that the circuit genes can be used as features to classify disease states, providing a bioinformatics solution to challenging diagnostic problems.

### 1.3.2 Comparative Analysis of Performance

The systems and methods of the present disclosure provide a scalable framework. It can infer regulatory circuits of TFs, chromatin regions, and genes with differential activities between contrast conditions or infer regulatory circuits with active chromatin regions and genes in a single condition. Because existing integrative methods can only be applied to single-condition data, to provide a comparative assessment of the performance of the systems and methods of the present disclosure, the present disclosure was restricted to the single-condition data analysis possible with existing methods.

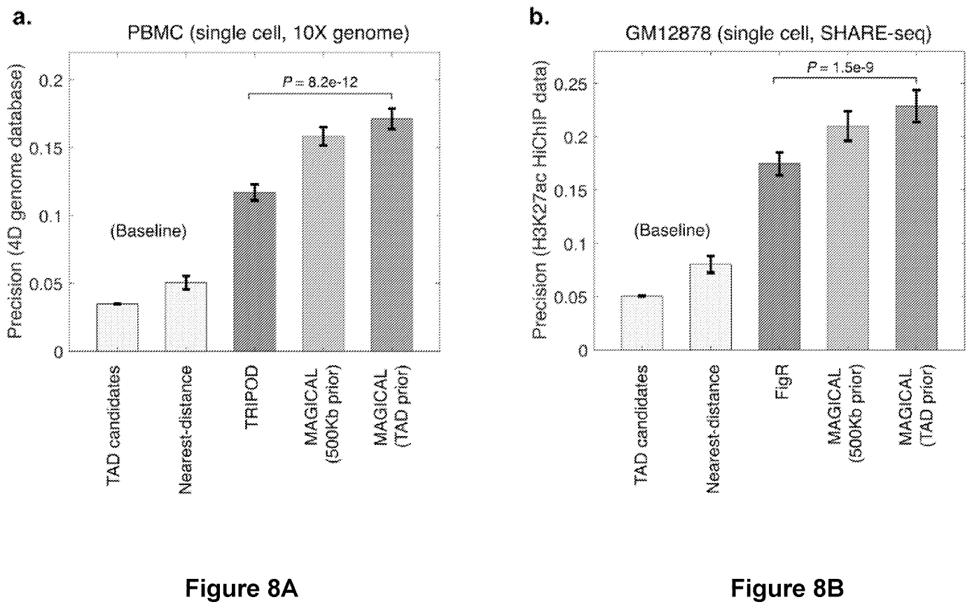

For peak-gene looping inference, the systems and methods of the present disclosure were compared to the TRIPOD11 and FigR methods, using the same benchmark single-cell multiome datasets as used by the authors reporting these methods. In the comparison of the systems and method of the present disclosure with TRIPOD using a 10× multiome single-cell dataset, inferred peak-gene loops made by the systems and method of the present disclosure showed significantly higher enrichment of experimentally observed chromatin interactions in blood cells in the 4DGenome database (Teng et al., 2015) (p-value<0.0001, two-side Fisher's exact test, FIG. 8A, where Magical-TAD prior represents the systems and methods of the present disclosure), the same validation data used by TRIPOD developers. The systems and methods of the present disclosure also significantly outperformed FigR on the application to a GM12878 SHARE-seq dataset (Ma et al., 2020). In that case, the peak-gene loops in MAGICAL-selected circuits had significantly higher enrichment of H3K27ac-centric chromatin interactions20 than did FigR (p-value<0.0001, two-side Fisher's exact test, FIG. 8B, where again, Magical-TAD prior represents the systems and methods of the present disclosure).

Because the framework of the systems and methods of the present disclosure unlike TRIPOD and FigR, used chromatin TAD boundaries as prior information, a determination was made as to whether the improvement in performance of the present disclosure illustrated in FIG. 8 resulted solely from this additional information. To investigate this, the systems and methods of the present disclosure eliminated the use of TAD boundaries and was modified, for this test, by assigning candidate linkages between peaks and genes within 500 Kb (a naïve distance prior). As shown in FIGS. 8A and 8B, even without the TAD prior information, the systems and methods of the present disclosure, now denoted Magical-500 Kb prior, still outperformed the competing methods (p-values<0.001, two-side Fisher's exact test). Overall, these results suggest that in addition to the benefit of priors, explicit modeling of signal and noise in both chromatin accessibility and gene expression data increased the accuracy of peak-gene looping identification.

### 1.3.3 MAGICAL Analysis of COVID-19 Single-Cell Multiomics Data

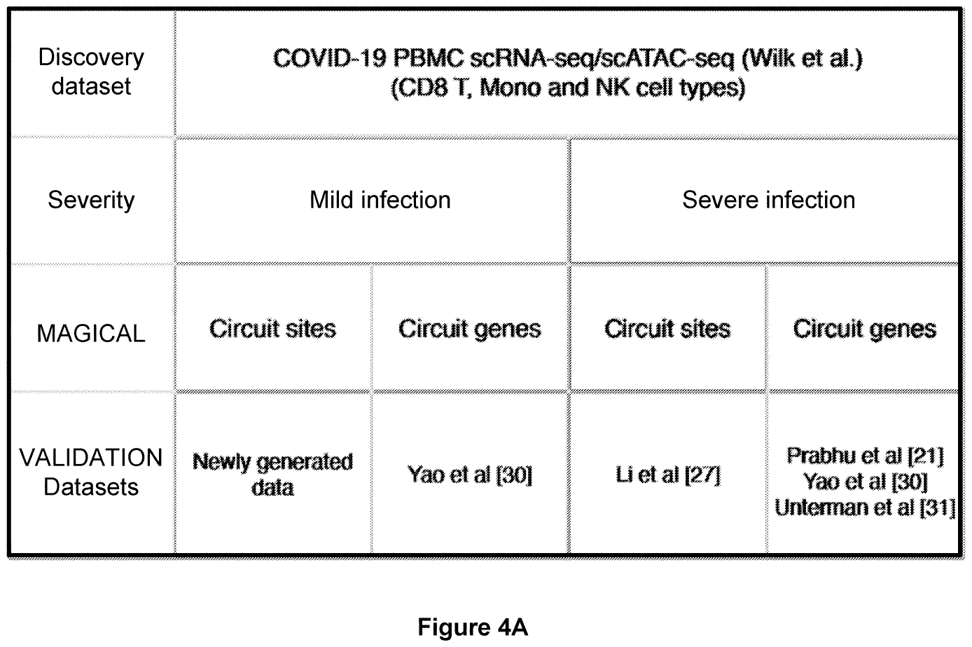

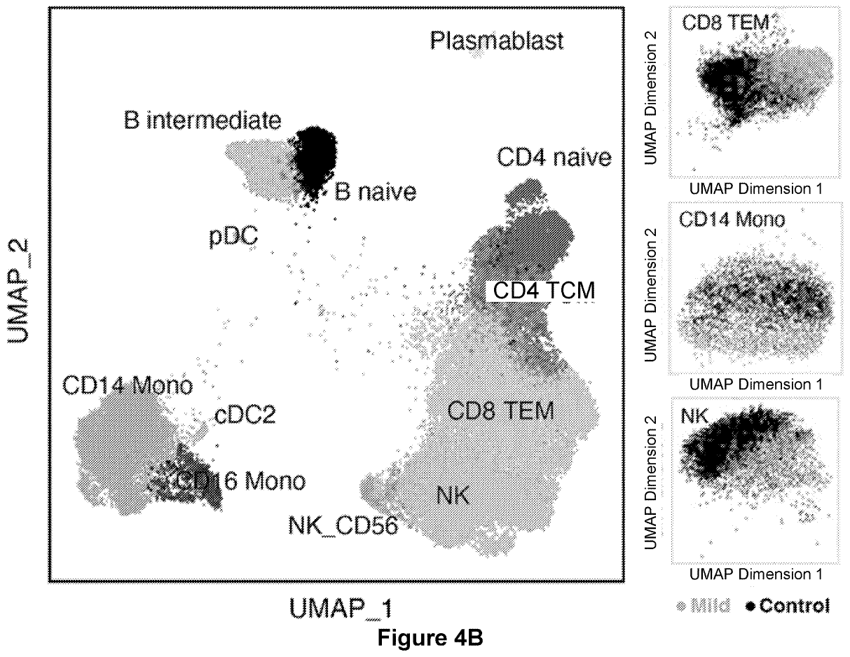

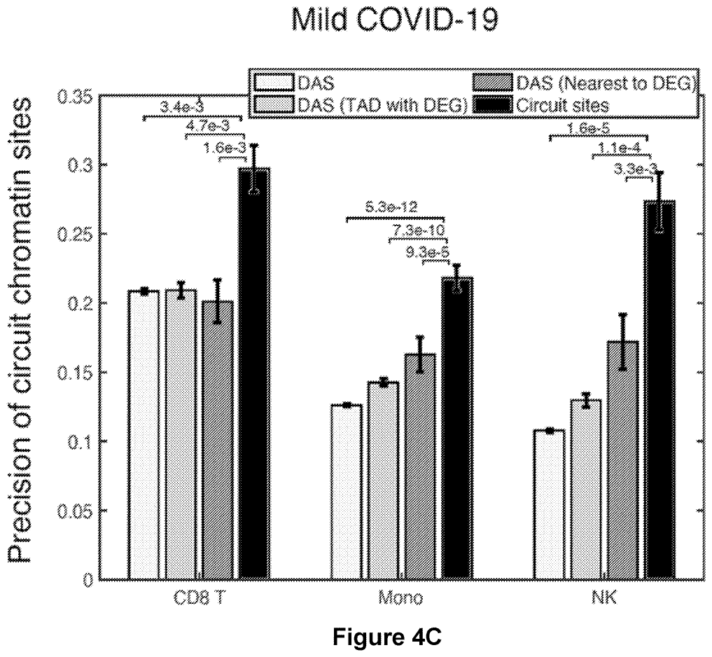

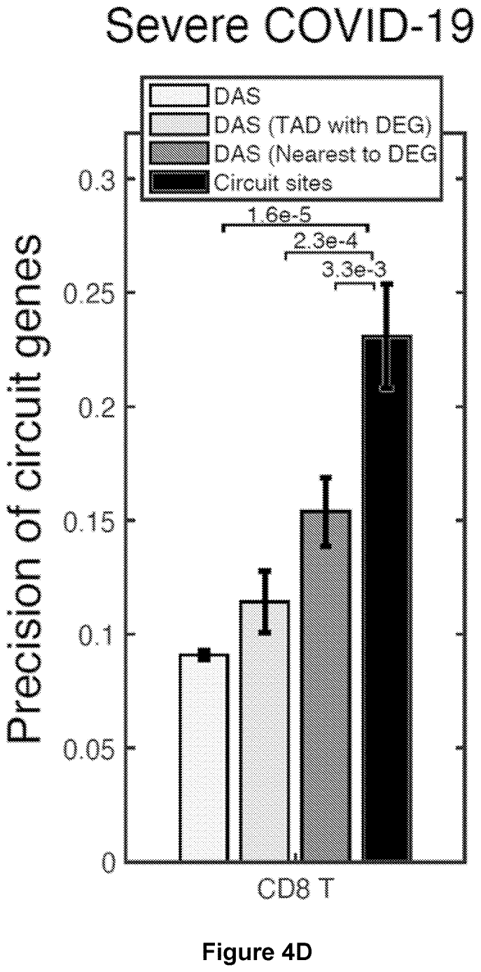

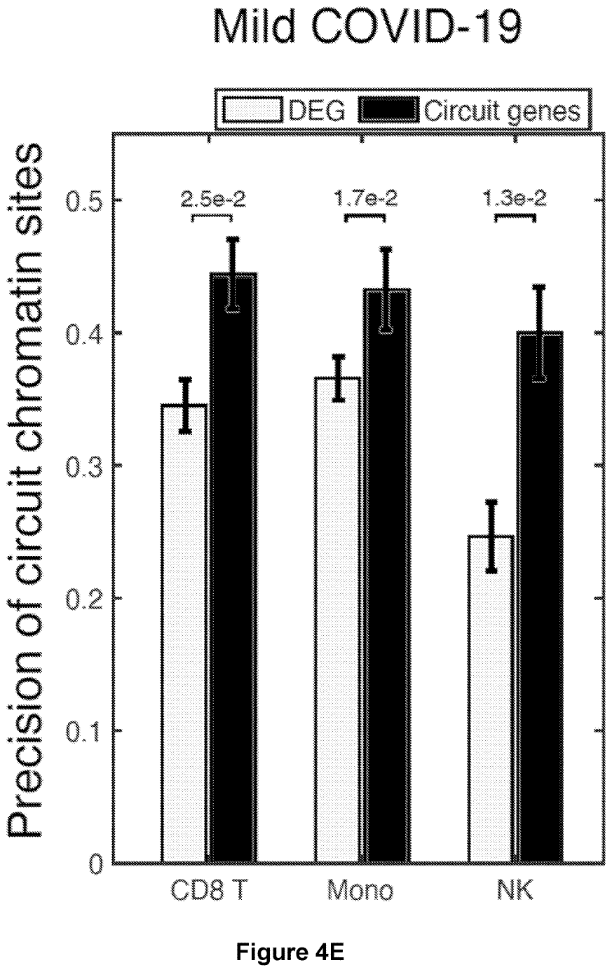

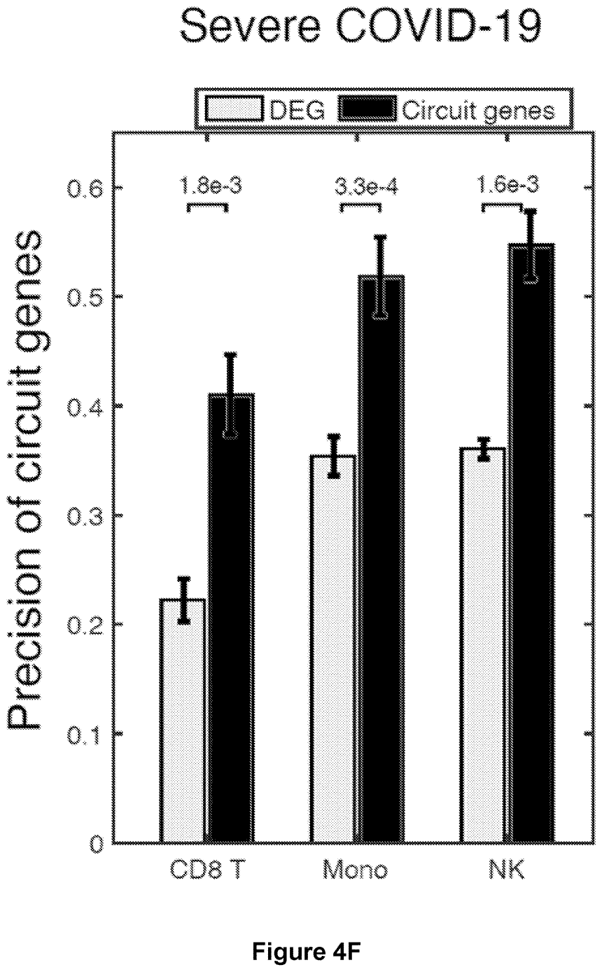

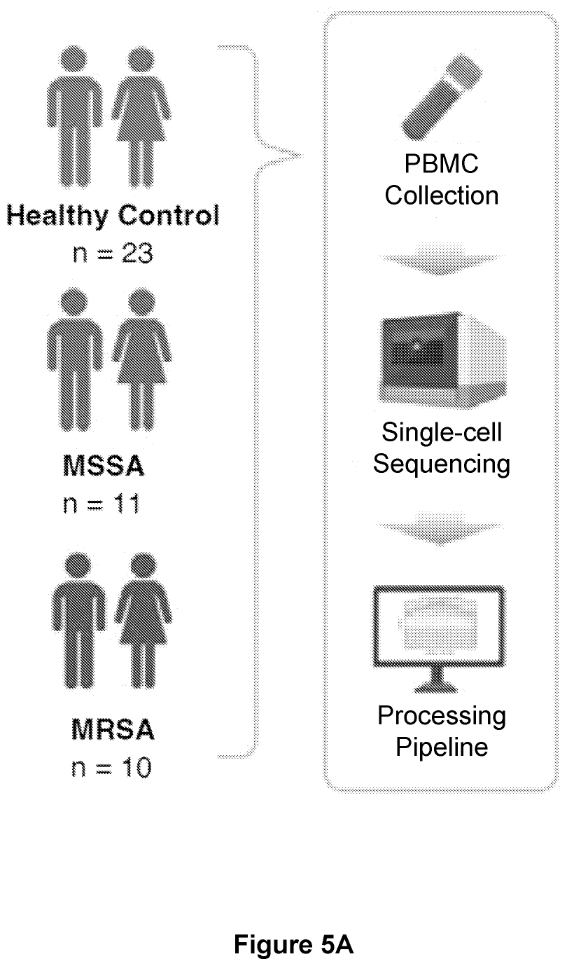

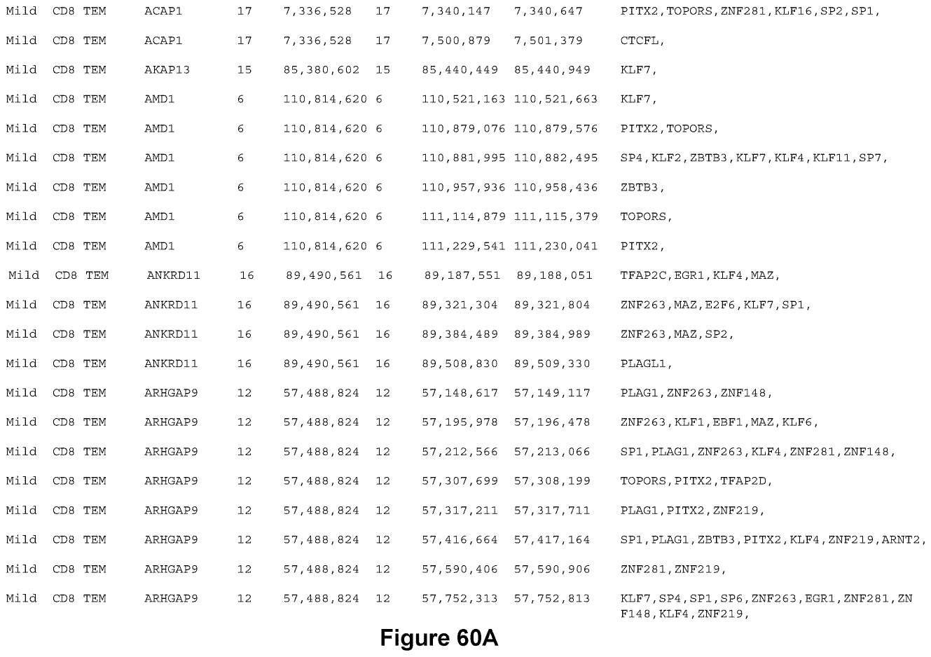

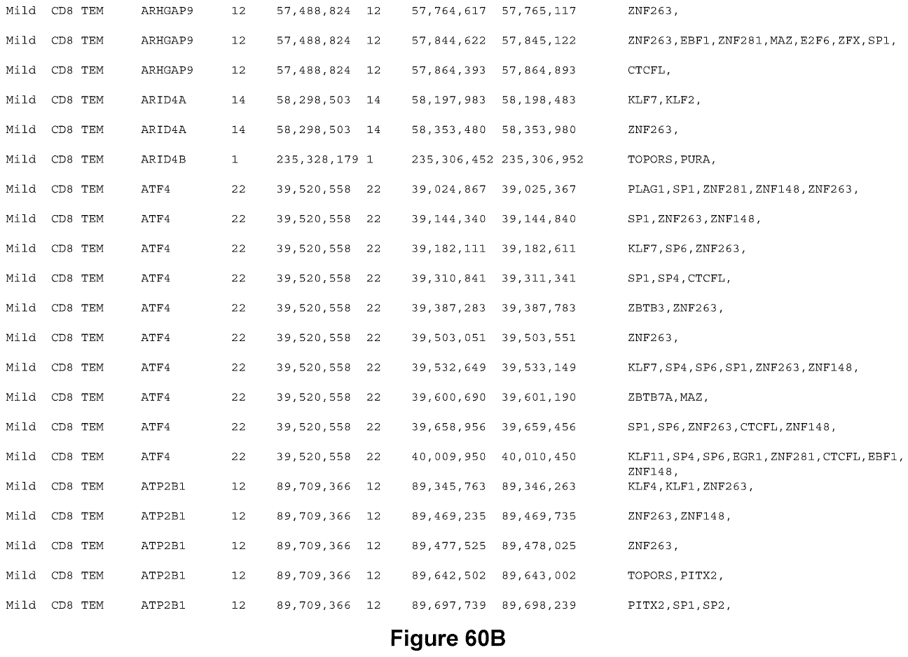

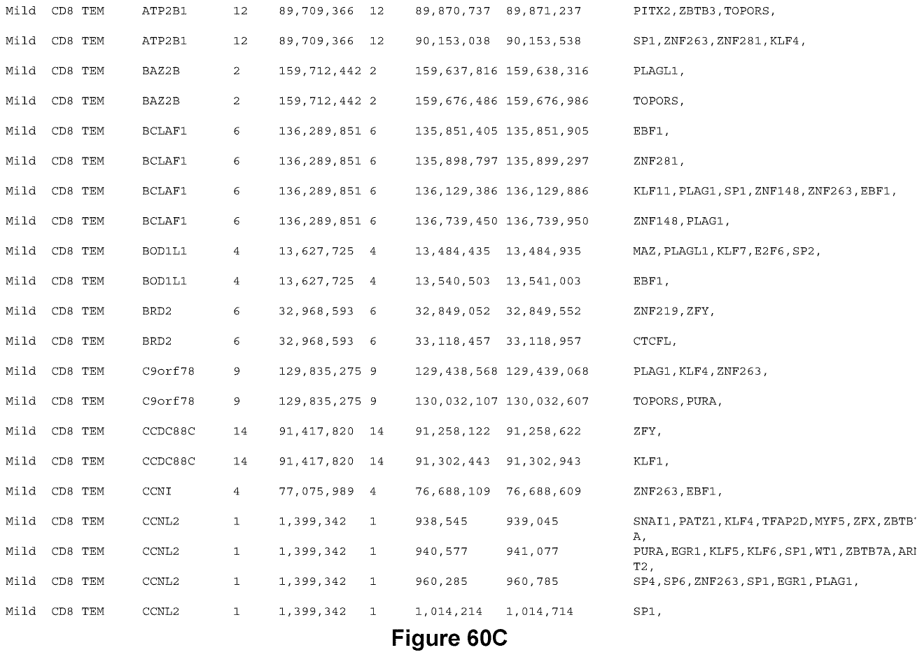

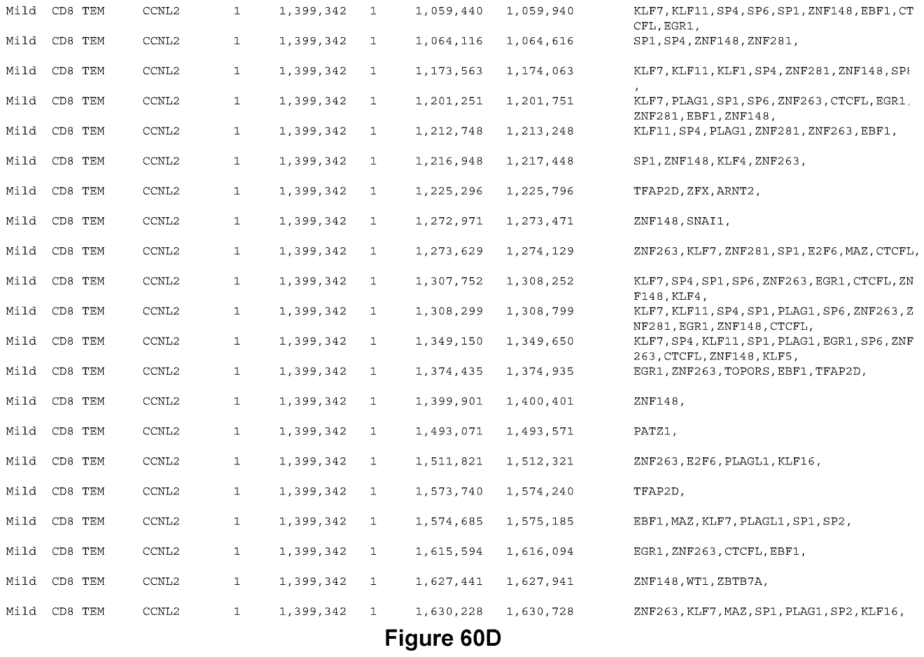

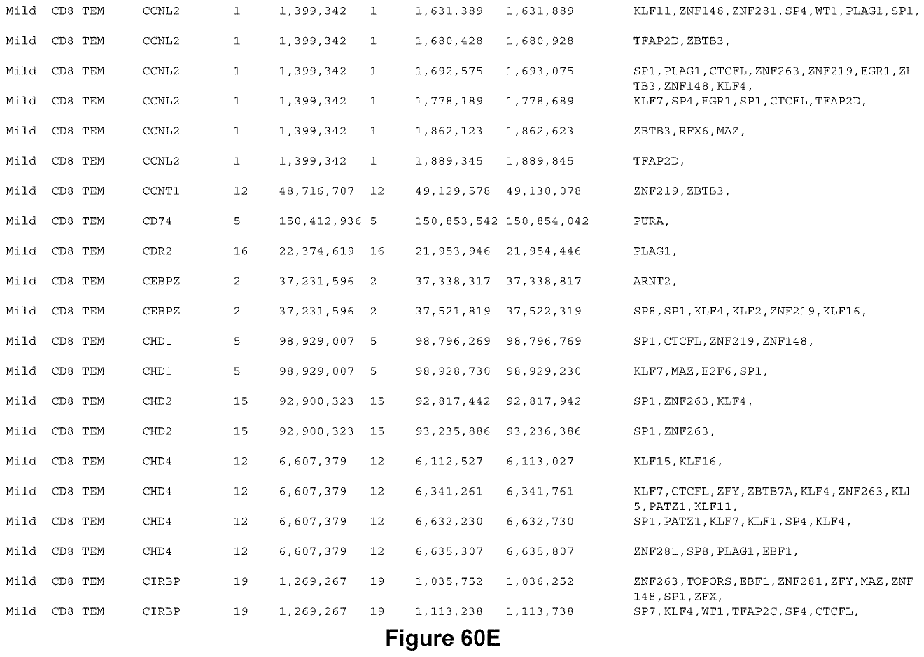

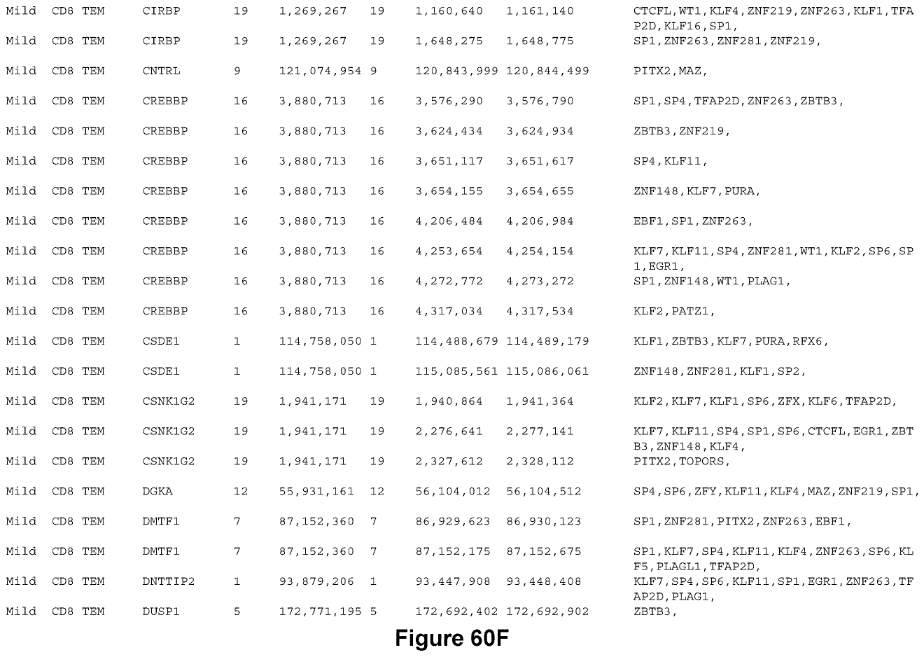

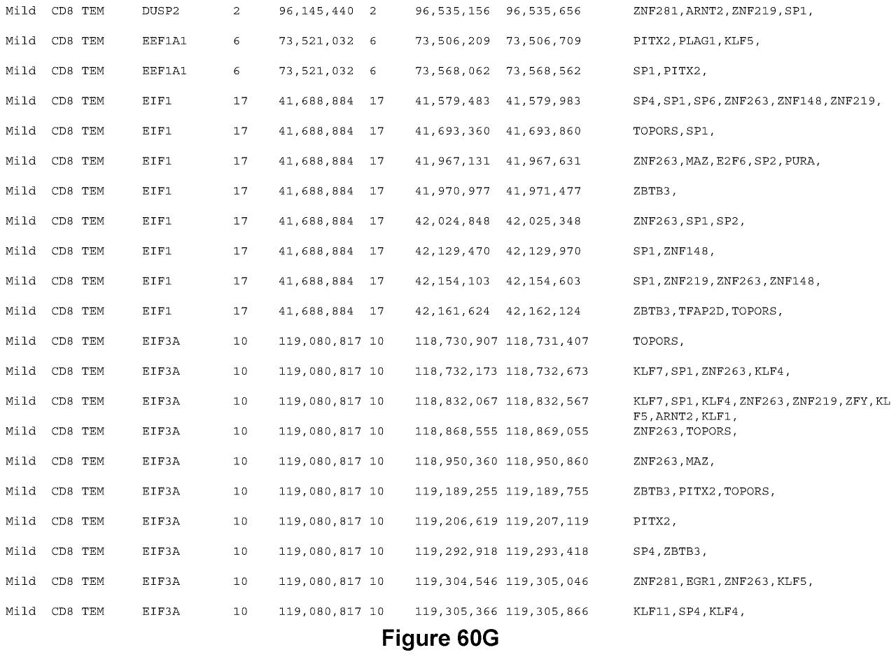

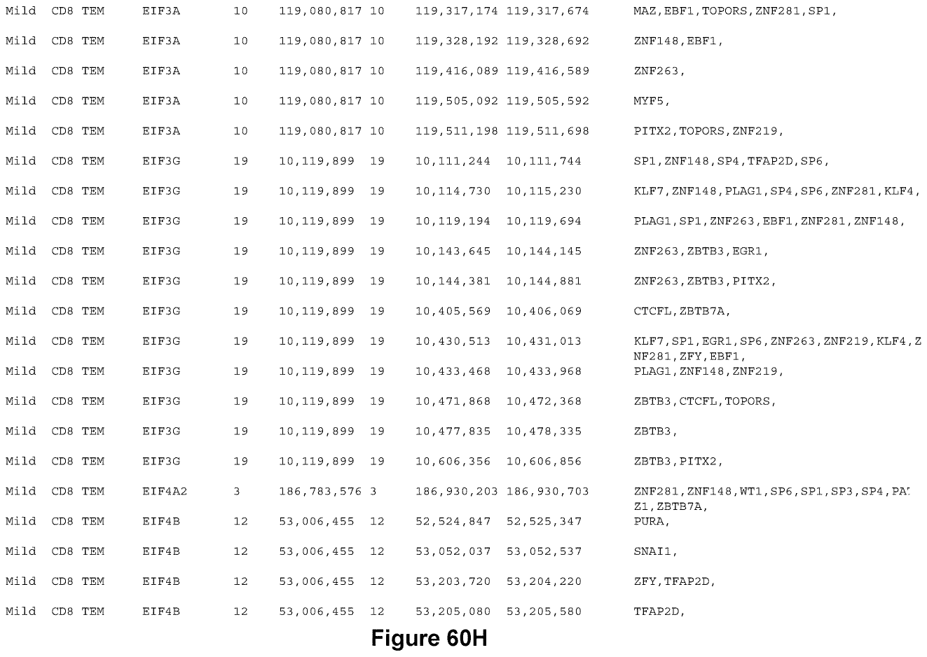

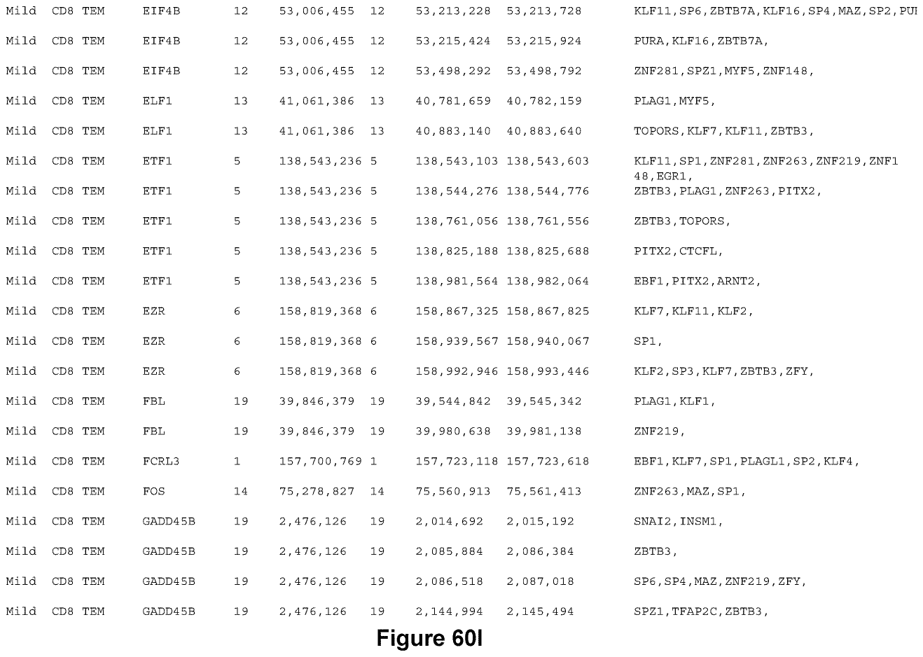

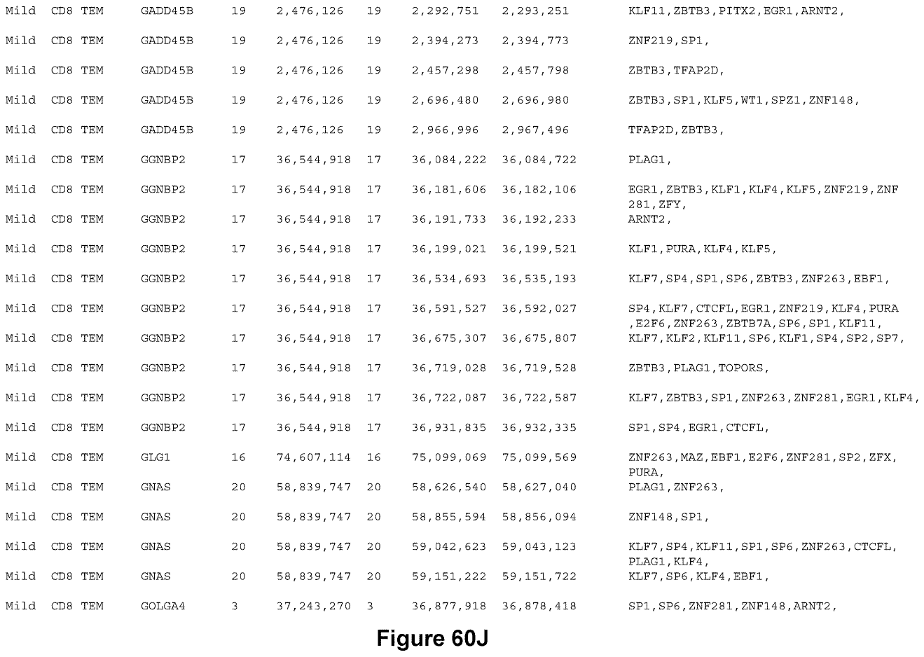

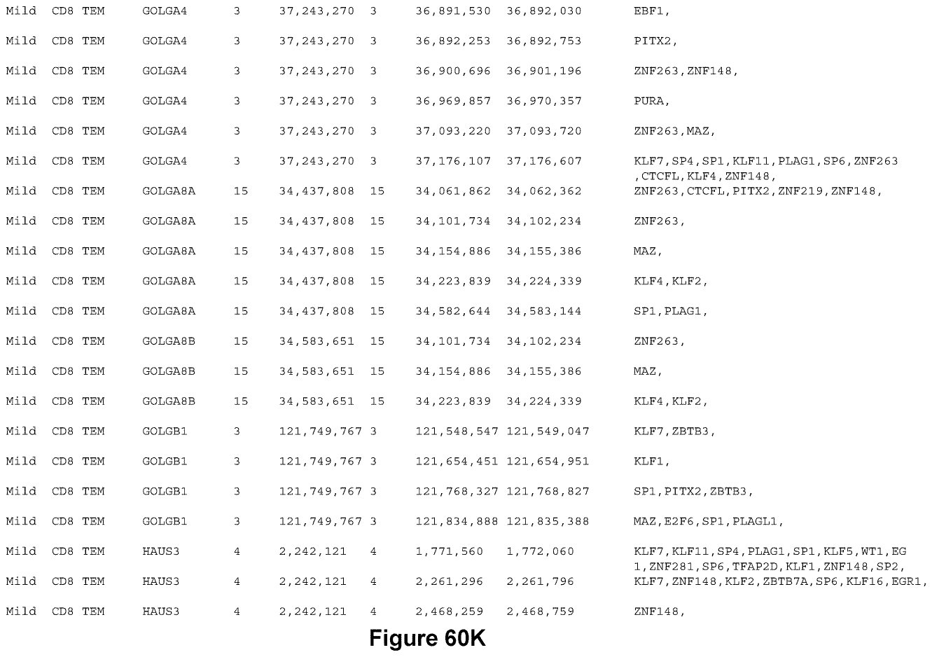

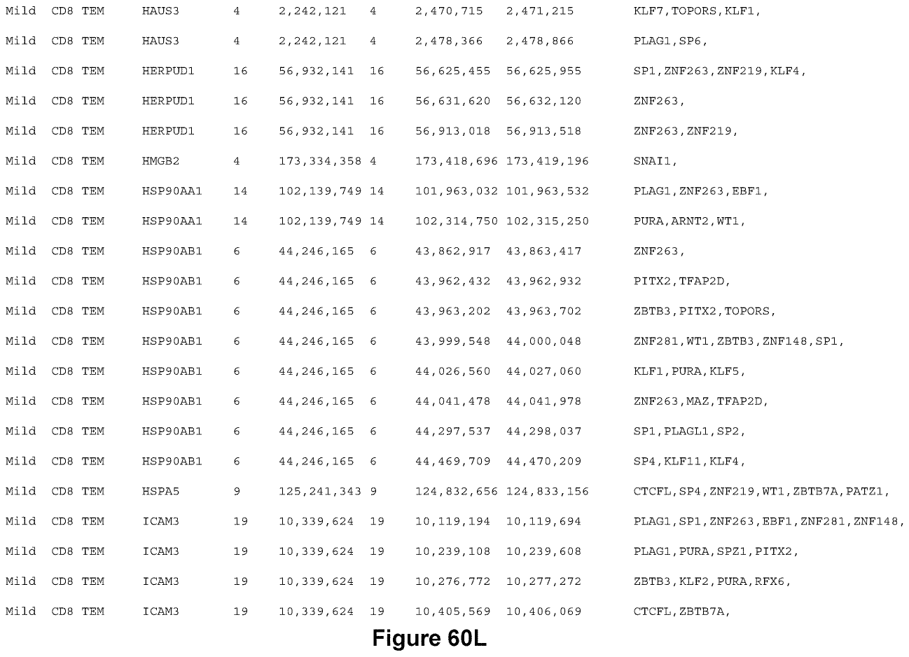

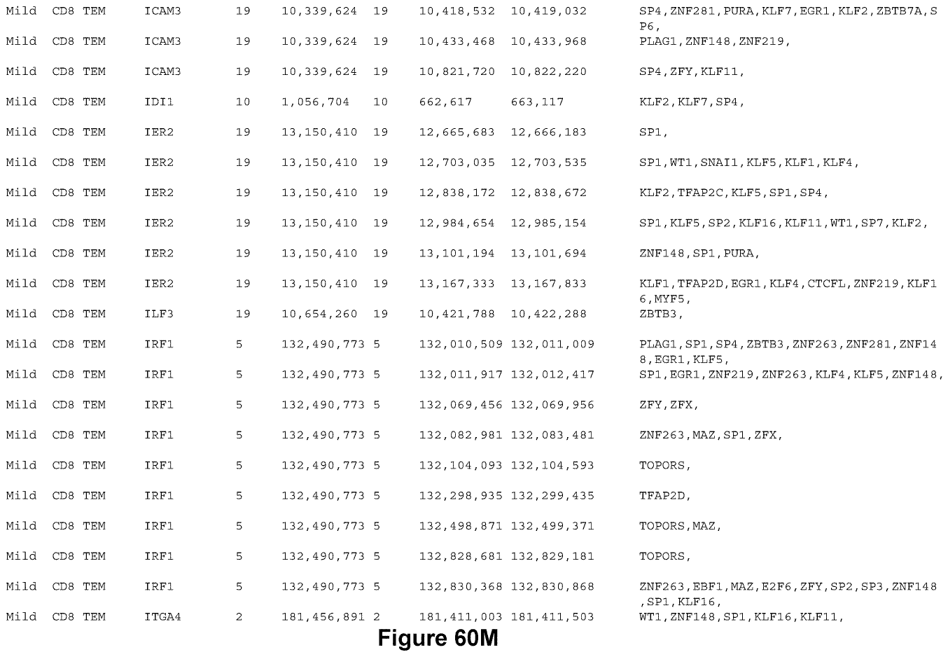

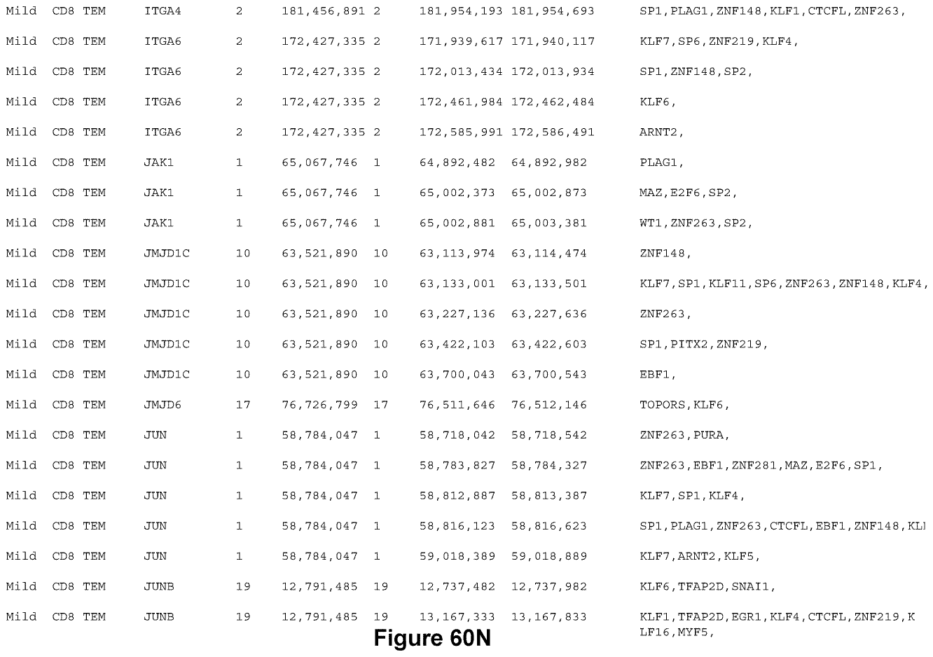

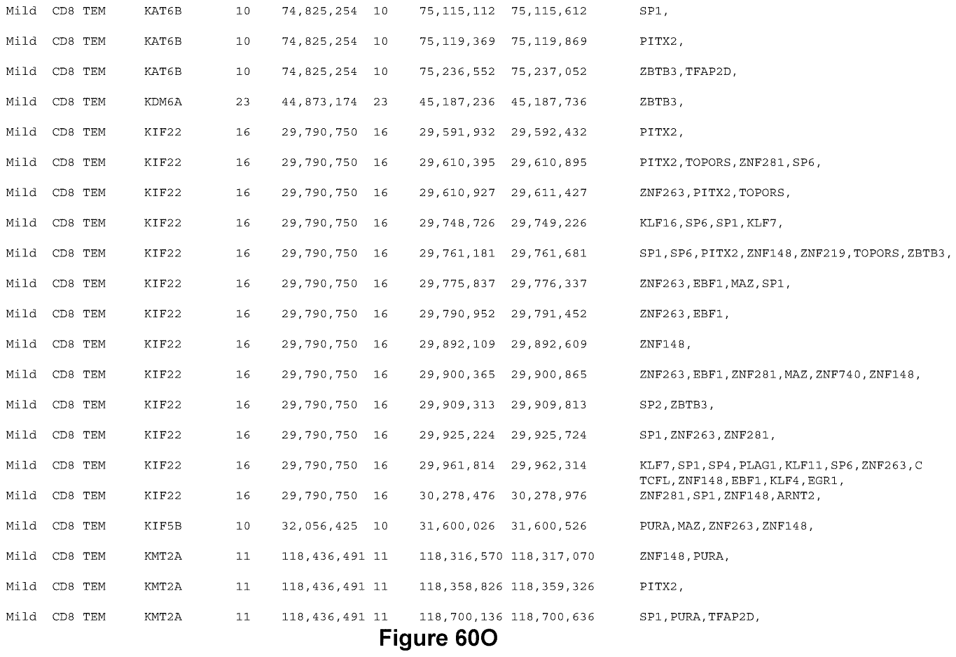

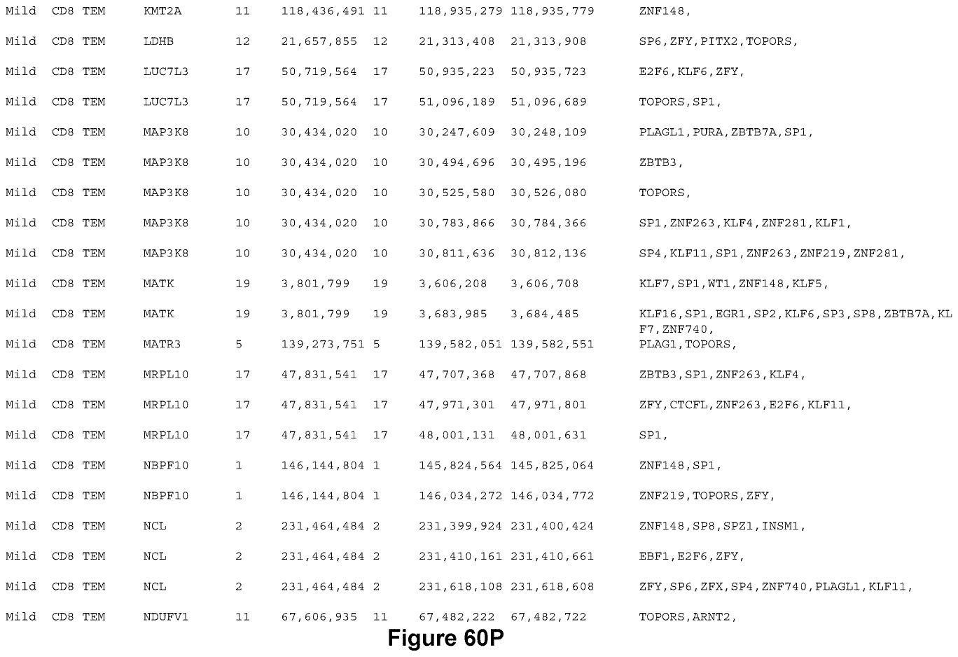

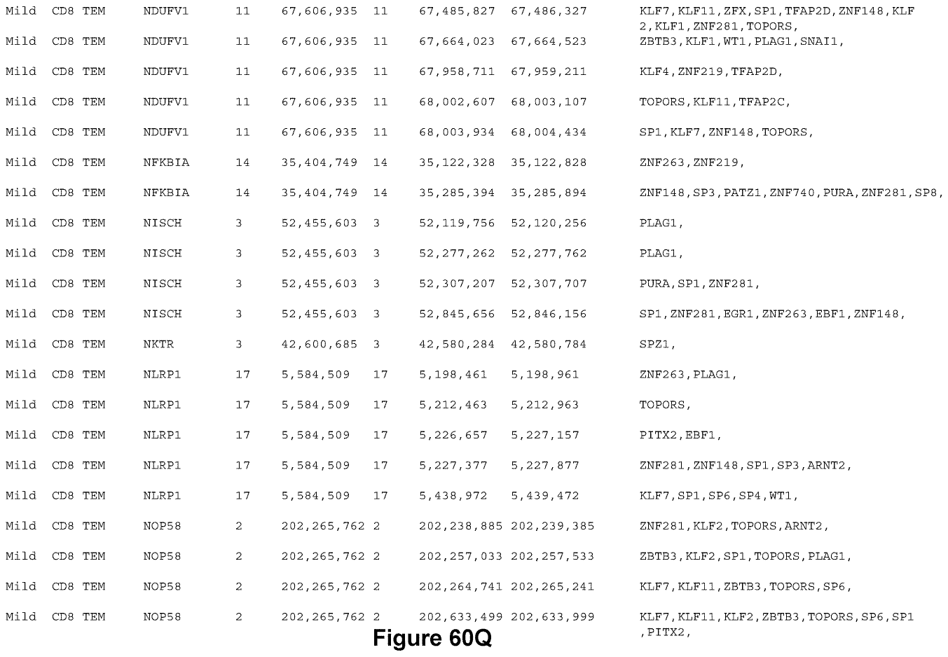

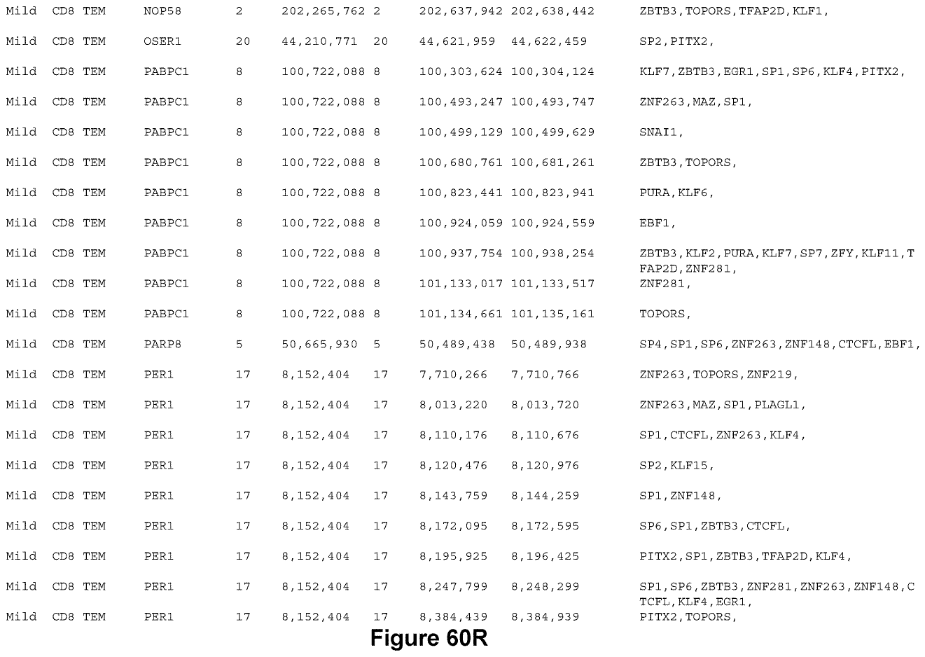

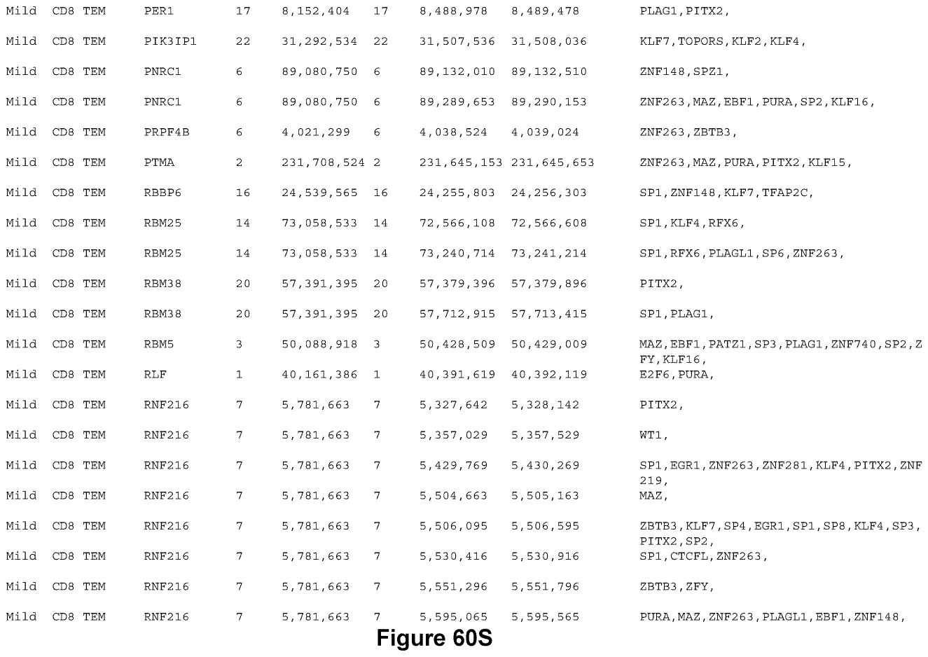

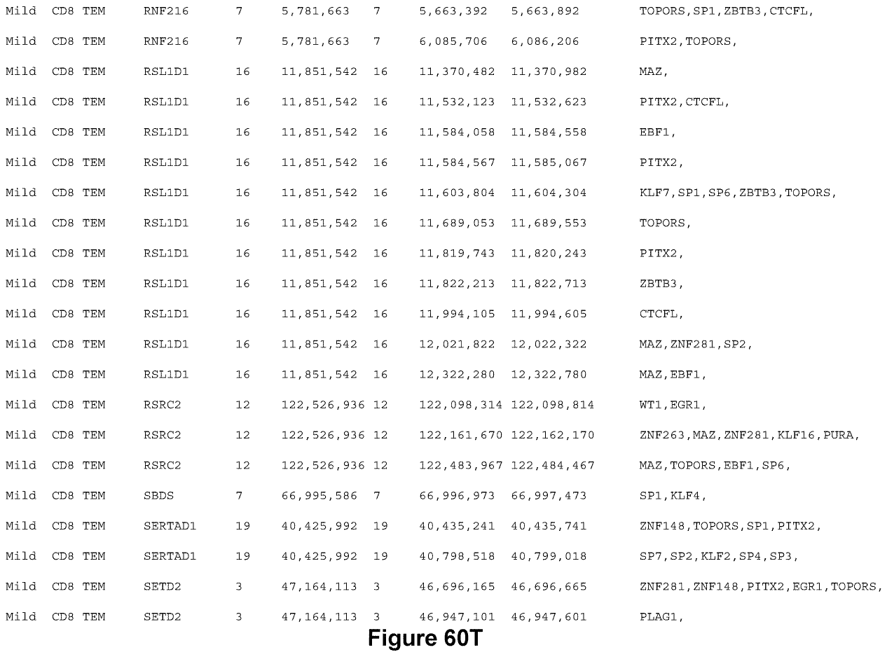

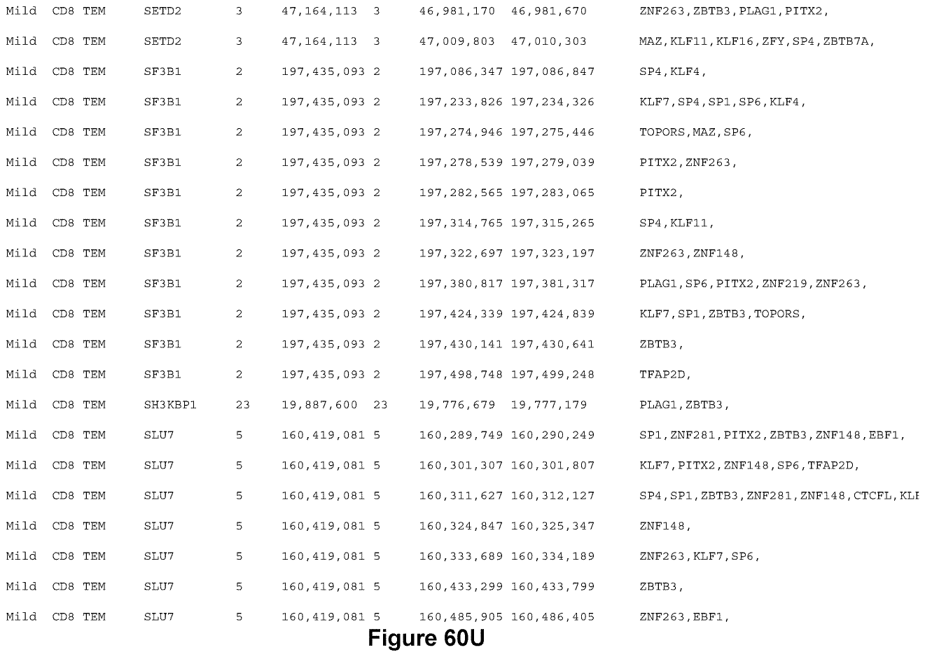

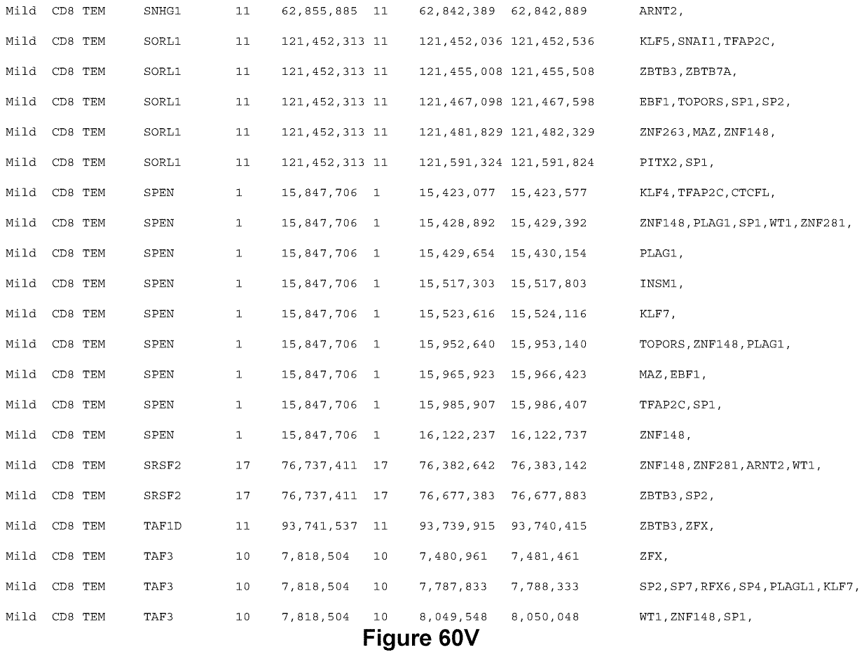

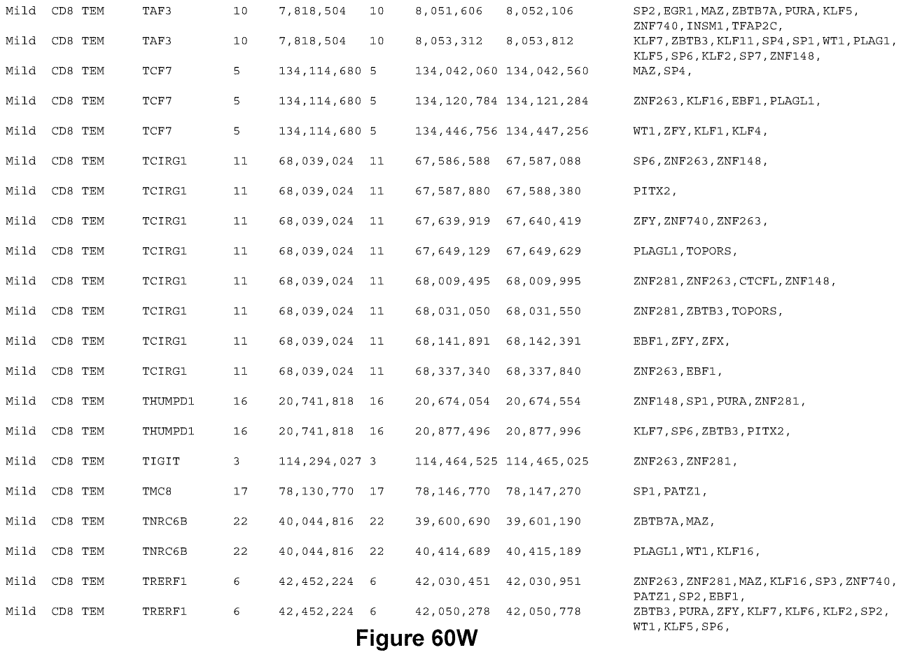

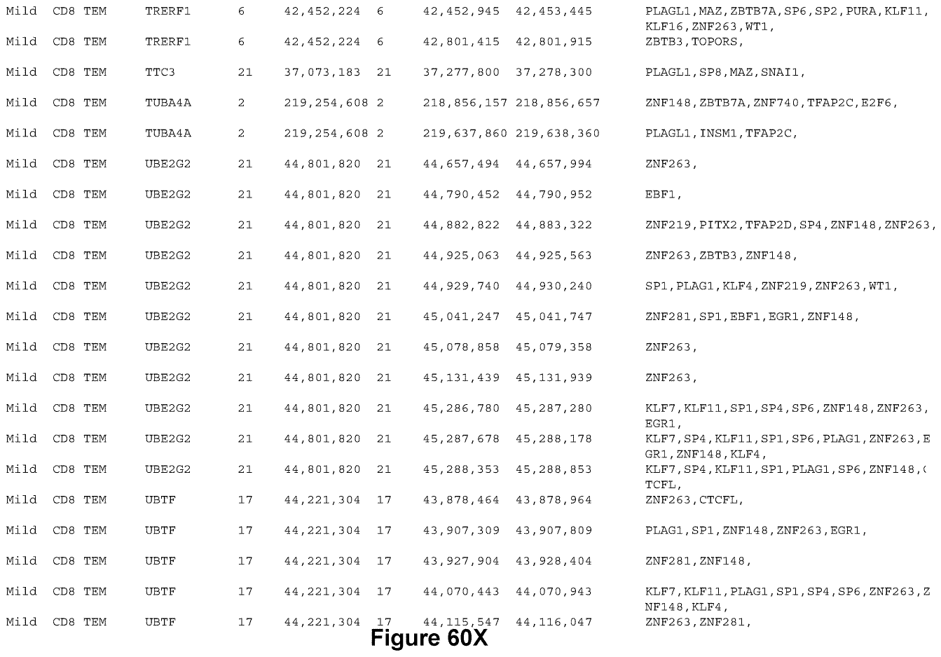

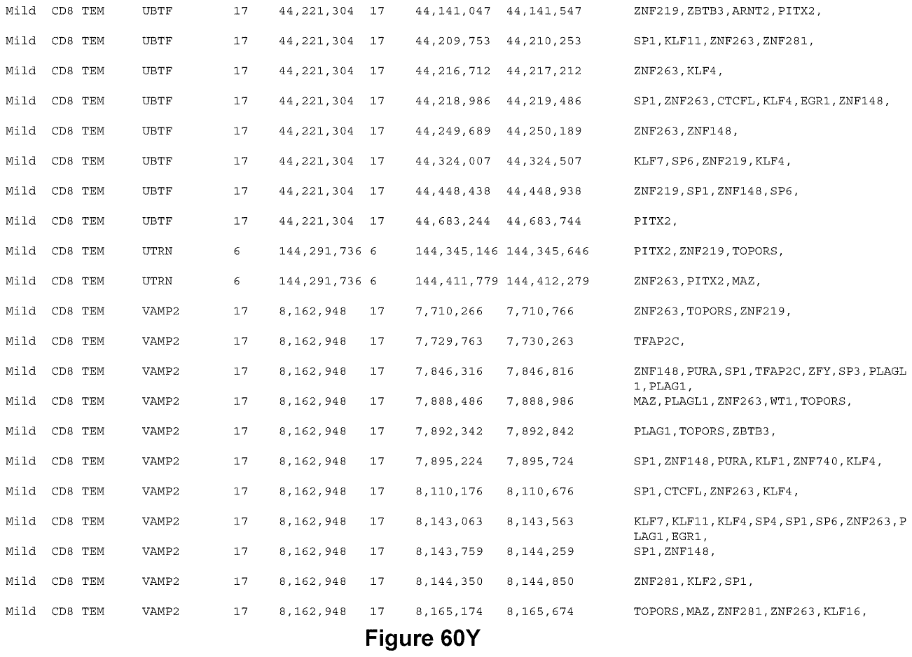

To demonstrate the accuracy of the primary application of the systems and methods of the present disclosure on contrast condition data to infer disease-modulated circuits, the systems and methods of the present disclosure were applied to sample-paired peripheral blood mononuclear cell (PBMC) scRNA-seq and scATAC-seq data from SARS-CoV-2 infected individuals and healthy controls. See Wilk et al., 2021 for details on this source data. Because immune responses in COVID-19 patients differ according to disease severity, (see Lucas et al., 2020; Mathew et al., 2020, each of which is hereby incorporated by reference in its entirety for all purposes), the systems and methods of the present disclosure inferred the regulatory circuits for mild and severe clinical groups separately. The chromatin sites and genes in the identified circuits were validated using newly generated and publicly available independent COVID-19 single-cell datasets (FIG. 8A). In some embodiments, the systems and methods of the present disclosure primarily focused on three cell types that have been found to show widespread gene expression and chromatin accessibility changes in response to SARS-CoV-2 infection: CD8 effector memory T (TEM) cells, CD14 monocytes (Mono), and natural killer (NK) cells. See Mathew et al., 2020; Schulte-Schrepping et al., 2020, each of which is hereby incorporated by reference in its entirety for all purposes. In total, 1,489 high confidence circuits (1,404 sites and 391 genes) were identified in these cell types for mild and severe clinical groups. FIG. 60 provides a subset of these 1489 high confidence circuits, section 1.5.12 below provides more details of the methods used. Also, further listings of the 1489 high confidence circuits not included FIG. 60 is found in Chen et al., 2023, “Mapping disease regulatory circuits at cell-type resolution from single-cell multiomics data,” Nature Computational Science, 3(7), pg. 644-657; Supplementary Table 1, which is hereby incorporated by reference in its entirety for all purposes. To confirm these circuit chromatin sites selected by the present disclosure for mild COVID-19, the systems and methods of the present disclosure generated an independent PBMC scATAC-seq dataset from six SARS-CoV-2-infected subjects with mild symptoms and three uninfected (PCR-negative) controls (FIG. 4B; Table 1.2).

TABLE 1.2
COVID-19 Patient and Control Samples

| Aliquot_ID | Sex | Age | Condition | scATACseq |
|---|---|---|---|---|
| 1855-T49 | M | <35 | Infection-Mild | PASS |
| | | | symptoms | |
| 2266-T32 | M | <35 | Infection-Mild | PASS |
| | | | symptoms | |
| 2528-T42 | M | <35 | Infection-Mild | PASS |
| | | | symptoms | |
| 2557-T32 | M | <35 | Infection-Mild | PASS |
| | | | symptoms | |
| 2624-T32 | M | <35 | Infection-Mild | PASS |
| | | | symptoms | |
| 2654-T35 | M | <35 | Infection-Mild | PASS |
| | | | symptoms | |
| 2773-T00 | M | <35 | Control | PASS |
| 2800-T00 | M | <35 | Control | PASS |
| 3000-T00 | M | <35 | Control | PASS |

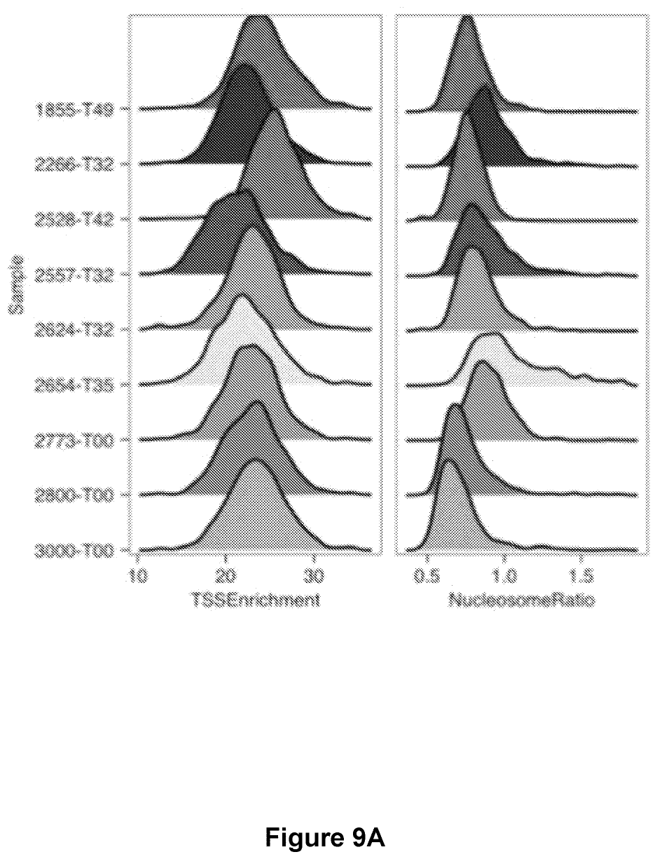

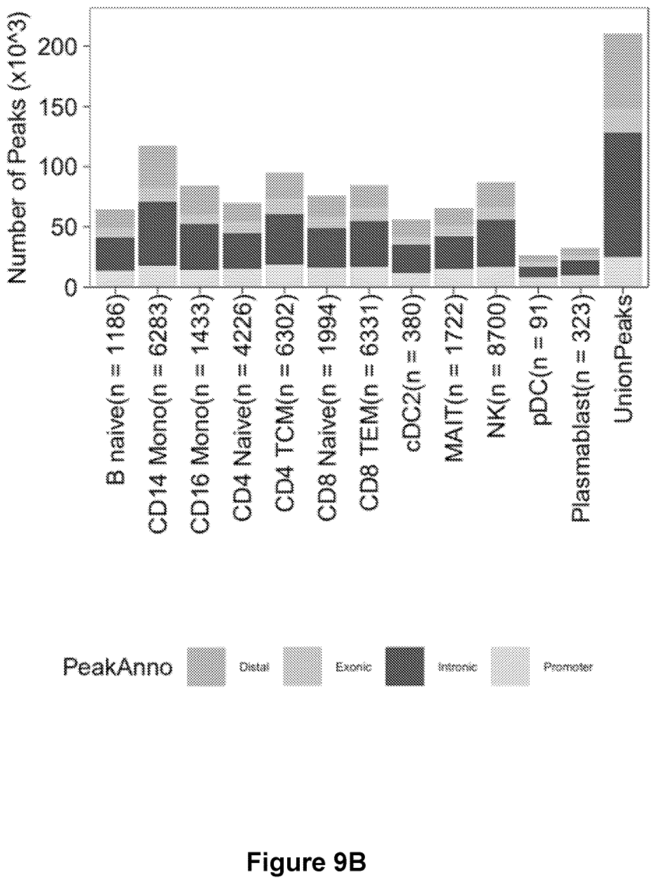

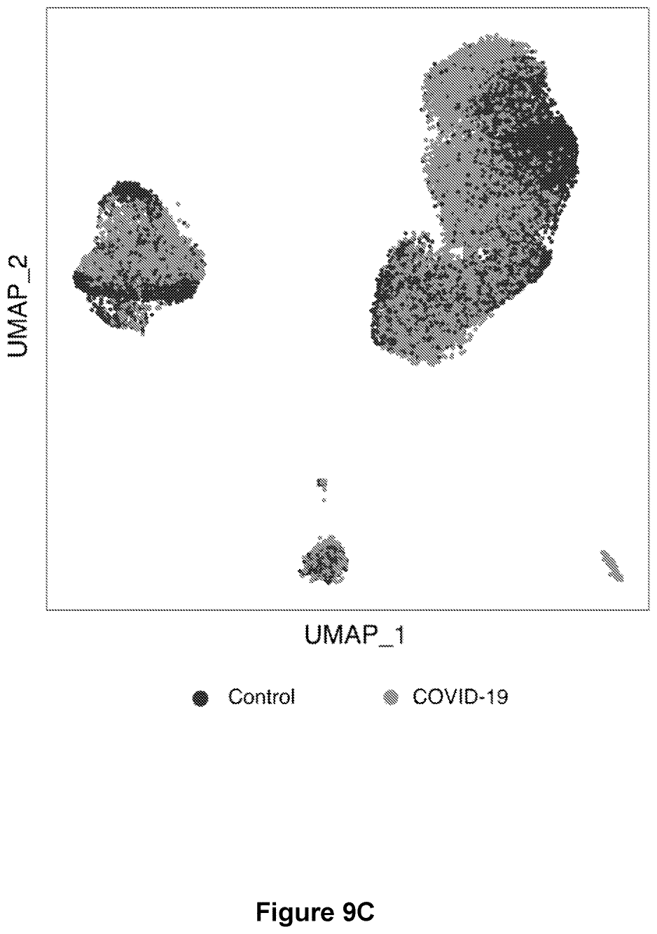

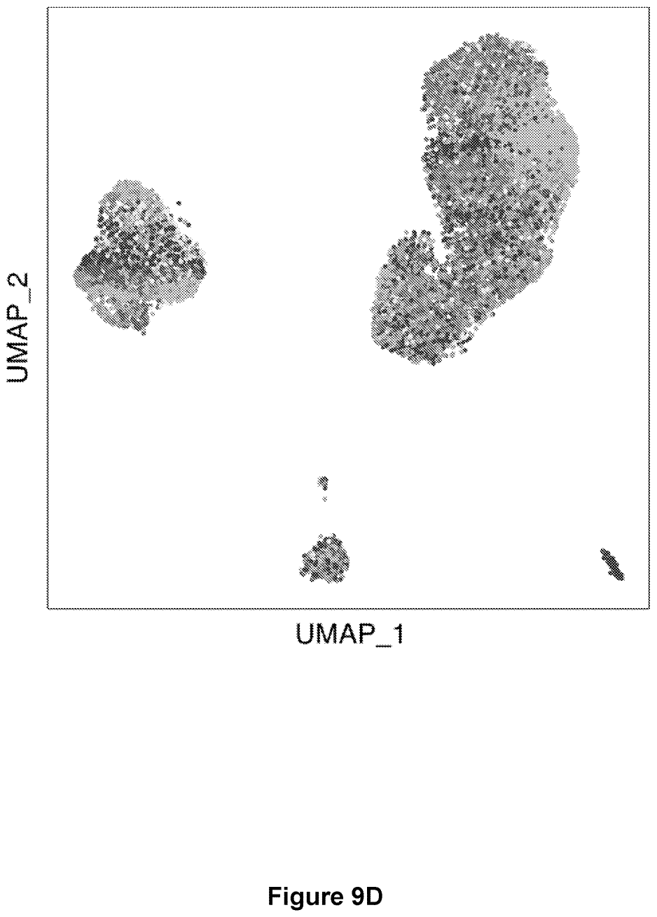

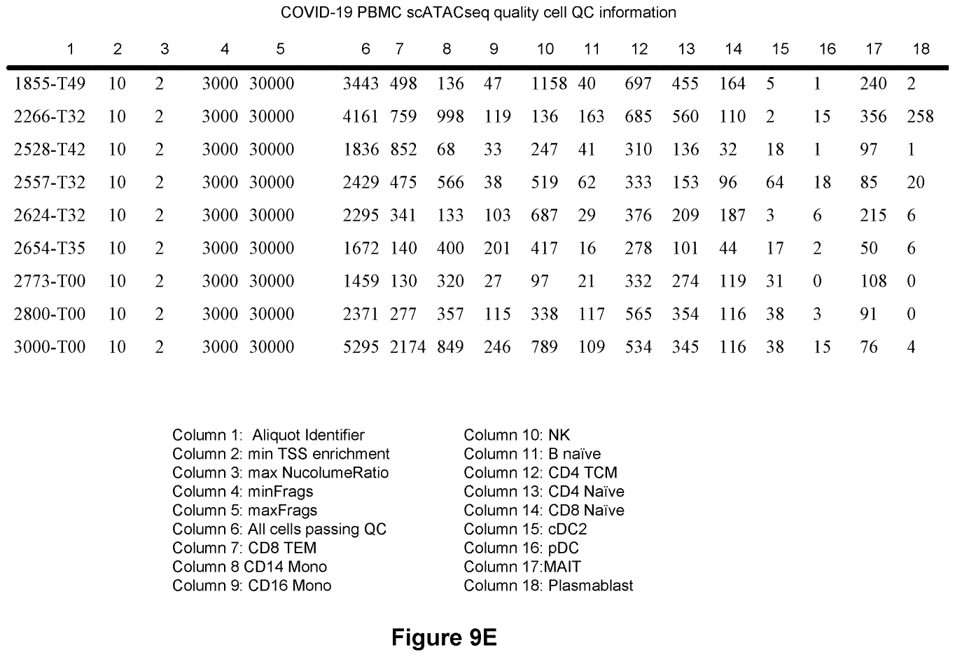

About 25,000 quality cells were selected after quality-control (QC) analysis. These cells were integrated, clustered and annotated using ArchR (FIGS. 9A-9E). See Granja et al., 2021, which is hereby incorporated by reference in its entirety for all purposes. Peaks were called from each cell type using MACS2. See Feng et al., 2012, which is hereby incorporated by reference in its entirety for all purposes. In total, 284,909 peaks were identified (Table 1.4). Details and information regarding Table 1.4 is found at Chen et al., 2023, “Mapping disease regulatory circuits at cell-type resolution from single-cell multiomics data,” Nature Computational Science 3, pp. 644-657; Supplementary Table 4, which is hereby incorporated by reference in its entirety for all purposes. For the three selected cell types, differential analysis between COVID-19 and control returned 3,061 sites for CD8 TEM, 1,301 sites for CD14 Mono, and 1,778 sites for NK (Table 1.5 and Section 1.5.13, below). Details and information regarding Table 1.5 is found at Chen et al., 2023, “Mapping disease regulatory circuits at cell-type resolution from single-cell multiomics data,” Nature Computational Science 3, pp. 644-657, Supplementary Table 5, which is hereby incorporated by reference in its entirety for all purposes. This produced three validation peak sets for mild COVID-19 infection. For severe COVID-19, an existing study focused on T cells identified specific chromatin activity changes with severe COVID-19 in CD8 T cells. See Li et al., 2021, which is hereby incorporated by reference in its entirety for all purposes. Their reported chromatin sites were used for validating the circuit chromatin sites identified in CD8 T cells. In all four validation sets, the precision (proportion of sites that are differential in the validation data) of the chromatin sites selected by the systems and methods of the present disclosure is significantly higher than the original DAS (p-values<0.001, two-side Fisher's exact test, FIGS. 4C and 4D).

When multiple potential chromatin regulatory loci are identified in the vicinity of a specific gene, it is commonly assumed that the locus closest to the transcriptional starting site (TSS) is likely to be the most important regulatory site. Challenging this assumption, however, are the results of experimental studies showing that genes may not be regulated by the nearest region. See Jung et al., 2019; and Chen et al., 2021, each of which is hereby incorporated by reference in its entirety for all purposes. Supporting the importance of more distal regulatory loci, the chromatin sites selected by the systems and methods of the present disclosure significantly outperformed the nearest DAS to the TSS of DEG or all DAS within the same TAD with DEG, and the improvement is substantial (precision is ~50% better with MAGICAL, p-values<0.05, two-side Fisher's exact test, FIGS. 4C and 4D).

To validate the circuit genes modulated by mild or severe COVID-19, the genes reported by external COVID-19 single-cell studies were used. See Yao et al., 2021; Unterman et al., 2022; and Arunachalam et al., 2020, each of which is hereby incorporated by reference in its entirety for all purposes. In total, six validation gene sets (three cell types for mild COVID-19 and three cell types for severe COVID-19) were collected. The precision of MAGICAL-selected circuit genes is significantly higher than that of original DEG in all validations (precision is ~30% better with MAGICAL, p-values<0.05, two-side Fisher's exact test, FIGS. 4E and 4F). These results confirmed the increased accuracy of disease association for both chromatin sites and genes in the regulatory circuits identified using the systems and methods of the present disclosure.

1.3.4 Analysis of S. aureus Single-Cell Multiomics Data

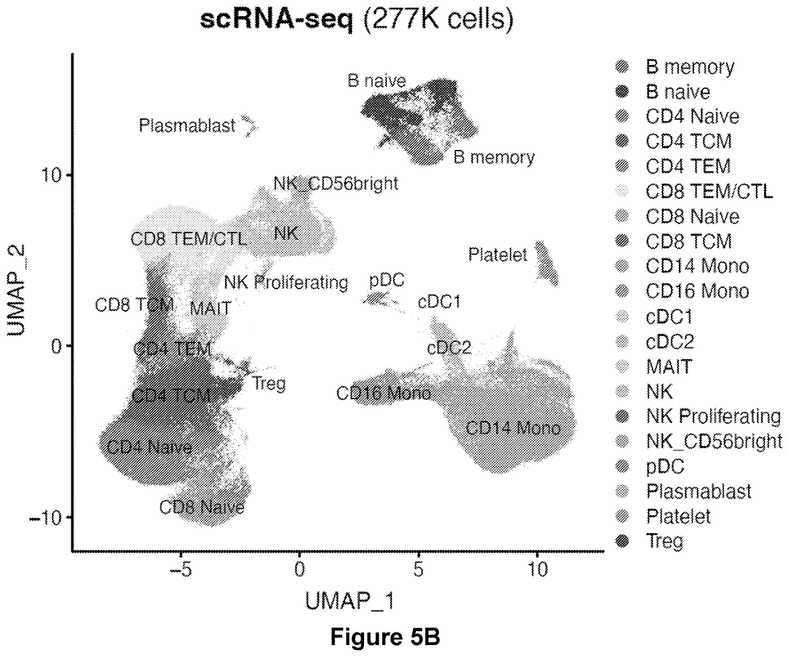

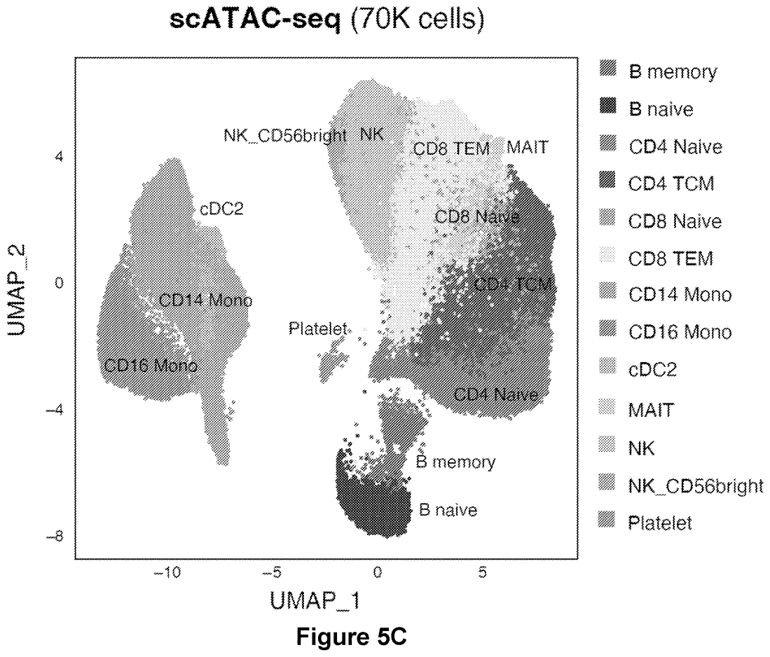

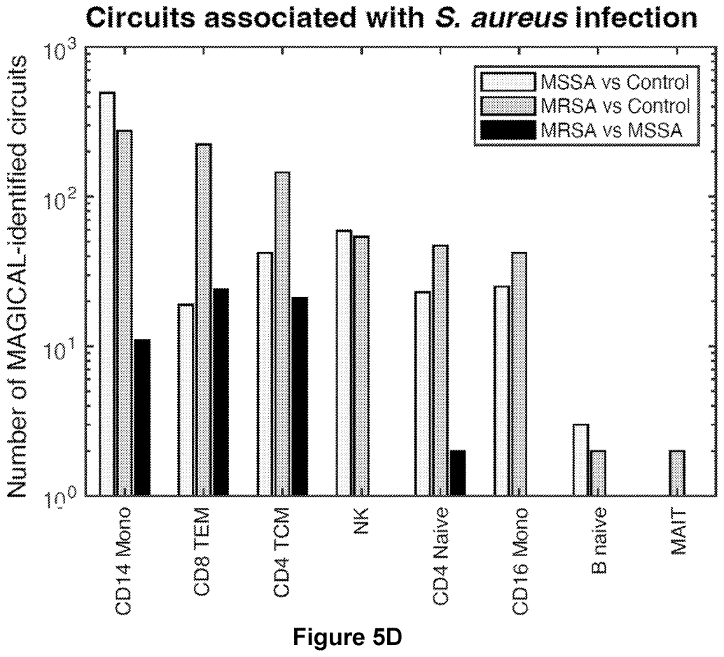

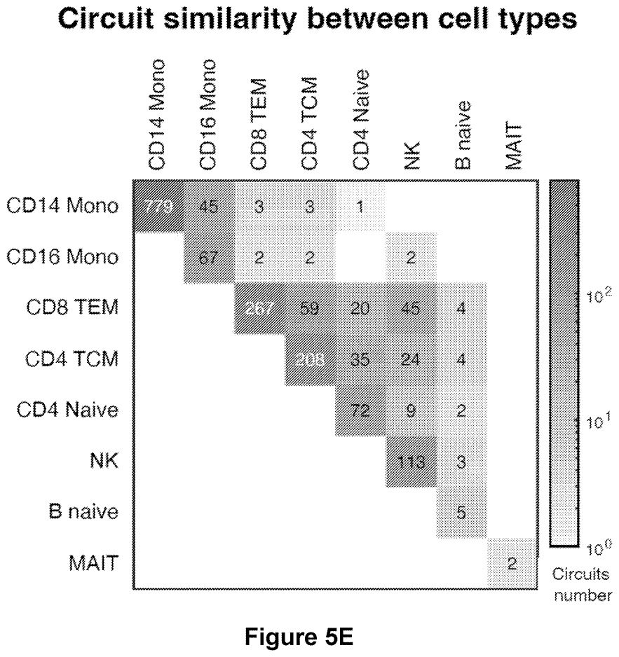

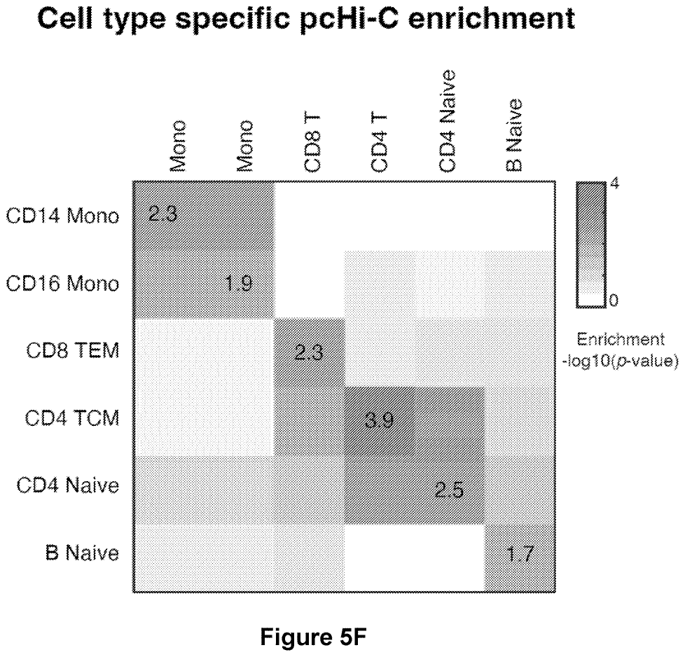

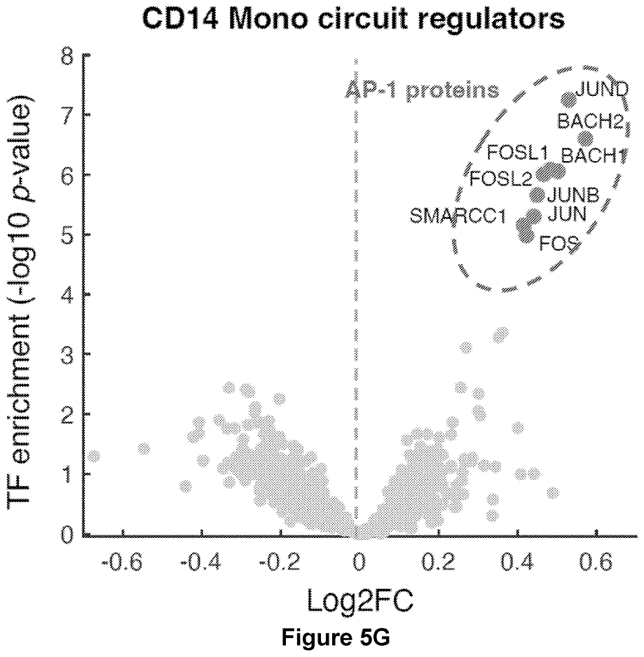

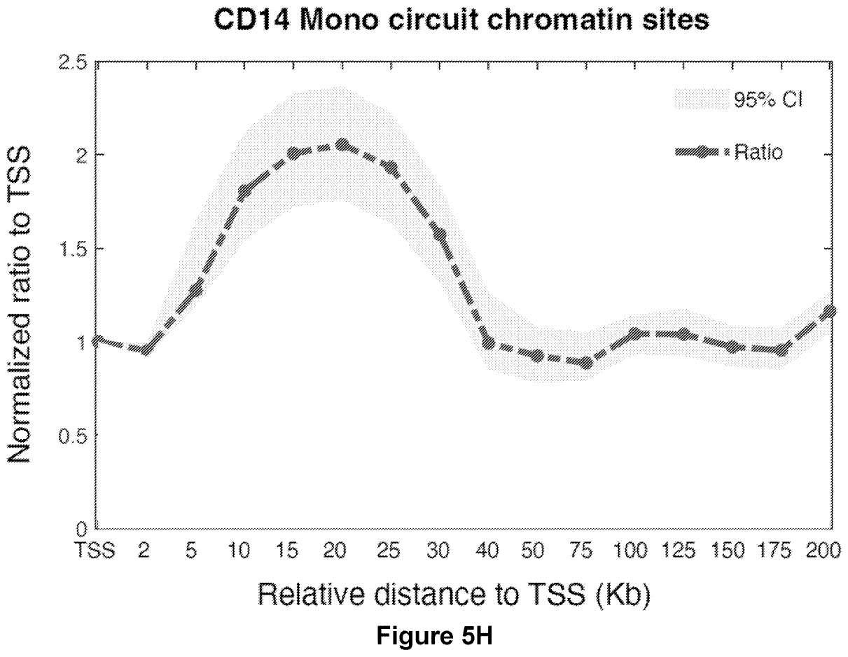

The systems and methods of the present disclosure were applied to the clinically important challenge of distinguishing methicillin-resistant (MRSA) and methicillin-susceptible S. aureus (MSSA) infections. See Magill et al., 2018; Tong et al., 2015; and Marquez-Ortiz et al., 2014. Paired scRNA-seq and scATAC-seq data were profiled using human PBMCs from adults who were blood culture positive for S. aureus, including 10 MRSA and 11 MSSA, and from 23 uninfected control subjects (FIG. 5A; Table 1.6).

TABLE 1.6
S.aureus infected and control PBMC samples

| Aliquot ID | Sex | Age | Condition | scRNAseq | scATACseq |
|---|---|---|---|---|---|
| AS08-09890 | M | <35 | Control | PASS | FAIL |
| AS09-13278 | M | <35 | Control | PASS | PASS |
| AS10-21035 | M | <35 | Control | PASS | FAIL |
| AS11-07049 | M | <35 | Control | PASS | FAIL |
| AS11-07881 | M | <35 | Control | PASS | FAIL |
| AS11-12162 | M | 35-65 | Control | PASS | FAIL |
| AS11-18755 | M | 35-65 | Control | PASS | PASS |
| AS13-08590 | M | 35-65 | Control | FAIL | PASS |
| AS13-13951 | M | <35 | Control | PASS | FAIL |
| AS14-00902 | M | 35-65 | Control | PASS | FAIL |
| AS14-03700 | M | 35-65 | Control | PASS | PASS |
| AS17-00144 | M | 35-65 | Control | PASS | PASS |
| AS17-02129 | M | 35-65 | Control | PASS | FAIL |
| AS18-00669 | M | <35 | Control | PASS | FAIL |
| BMI0037-M03 2 | F | <35 | Control | PASS | FAIL |
| BMI0040-M03 2 | M | <35 | Control | PASS | FAIL |
| BMI0093-M03 2 | F | 35-65 | Control | PASS | PASS |
| BMI0094-M03 2 | M | <35 | Control | PASS | PASS |
| BMI0095-M03 2 | M | 35-65 | Control | PASS | PASS |
| BMI0099-M03 2 | F | 35-65 | Control | PASS | PASS |
| BMI0101-M03 2 | M | <35 | Control | PASS | PASS |
| BMI0102-M03 2 | M | <35 | Control | PASS | FAIL |
| BWJ0023-M03 2 | F | <35 | Control | PASS | PASS |
| DU19-01S0003453 | F | <35 | MSSA | PASS | PASS |
| DU19-01S0003462 | F | <35 | MSSA | PASS | PASS |
| DU19-01S0003464 | F | 35-65 | MSSA | PASS | PASS |
| DU19-01S0003466 | M | <35 | MSSA | PASS | PASS |
| DU19-01S0003482 | M | 35-65 | MSSA | PASS | PASS |
| DU19-01S0003492 | M | 35-65 | MSSA | PASS | PASS |
| DU19-01S0003507 | F | 35-65 | MSSA | PASS | PASS |
| DU19-01S0003509 | F | 35-65 | MSSA | PASS | PASS |
| DU19-01S0003515 | M | 35-65 | MSSA | PASS | PASS |
| DU19-01S0003527 | F | 35-65 | MSSA | PASS | PASS |
| DU19-01S0003542 | M | 35-65 | MRSA | PASS | PASS |
| DU19-01S0003549 | F | >65 | MRSA | PASS | PASS |
| DU19-01S0003978 | F | <35 | MRSA | PASS | PASS |
| DU19-01S0003987 | F | 35-65 | MRSA | PASS | PASS |
| DU19-01S0003989 | F | <35 | MRSA | PASS | PASS |
| DU19-01S0003992 | M | >65 | MRSA | PASS | PASS |
| DU19-01S0003994 | M | 35-65 | MRSA | PASS | PASS |
| DU19-01S0004011 | F | 35-65 | MRSA | PASS | PASS |
| DU19-01S0004013 | F | 35-65 | MRSA | PASS | PASS |
| DU19-01S0004015 | M | >65 | MRSA | PASS | PASS |
| DU19-01S0004017 | M | >65 | MSSA | PASS | PASS |

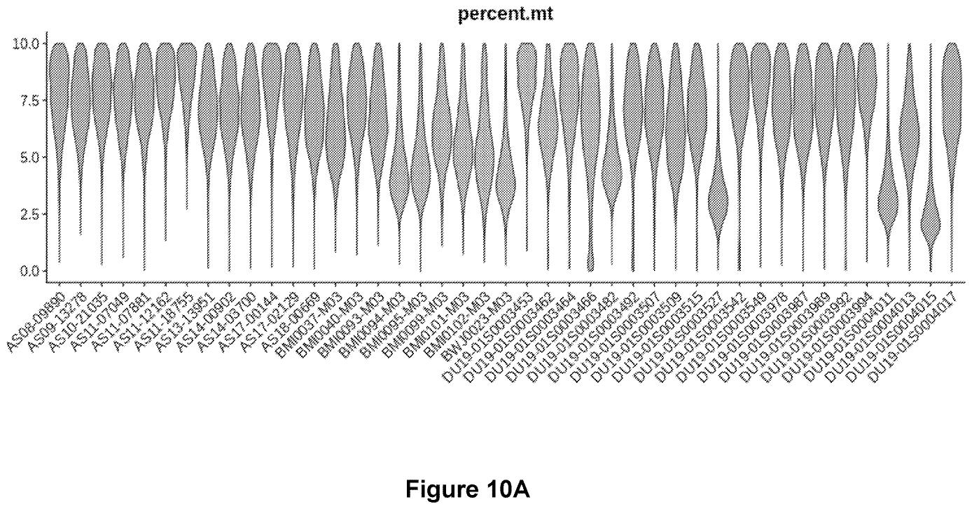

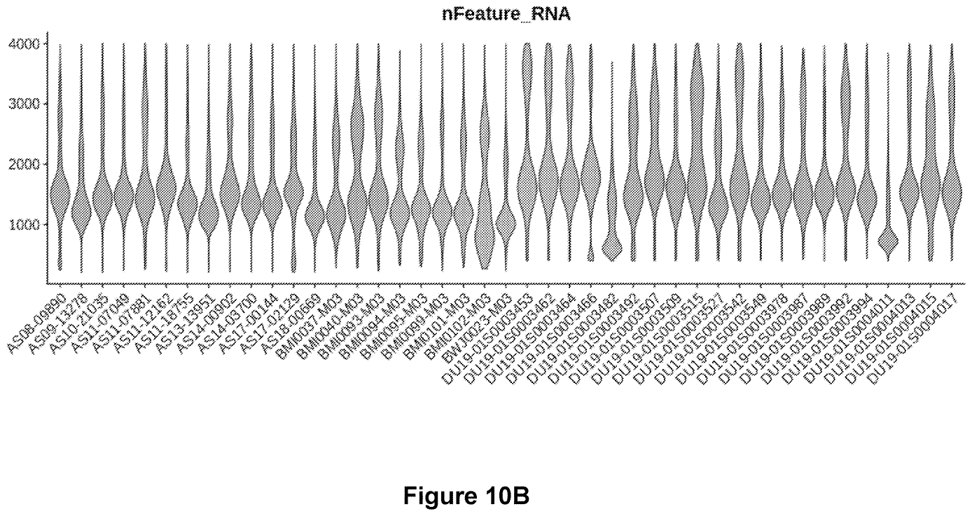

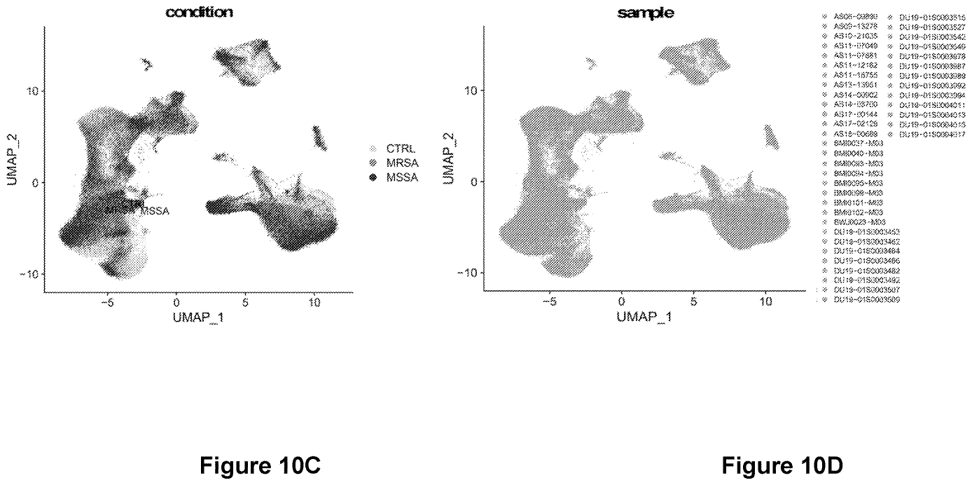

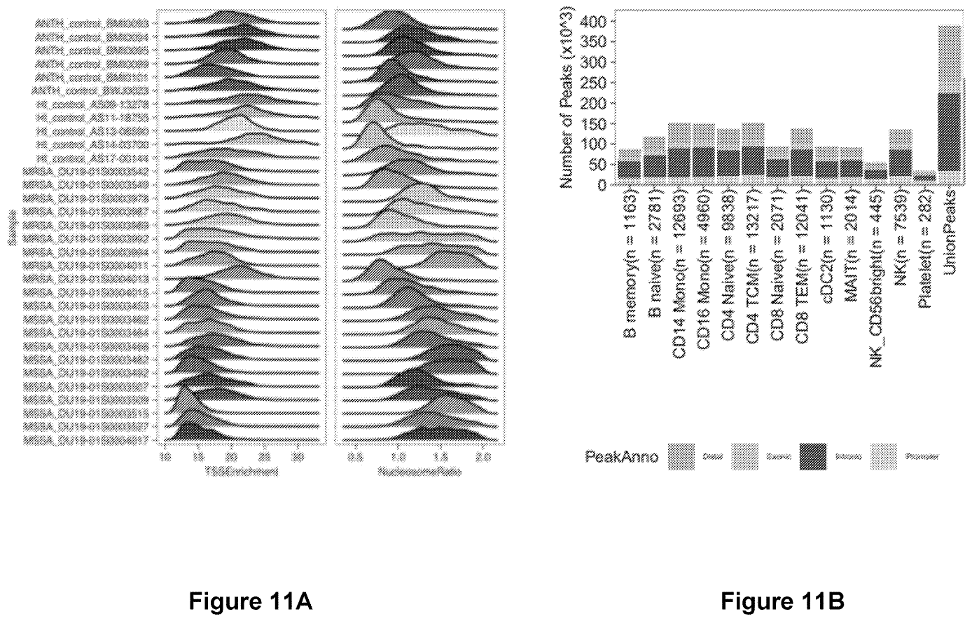

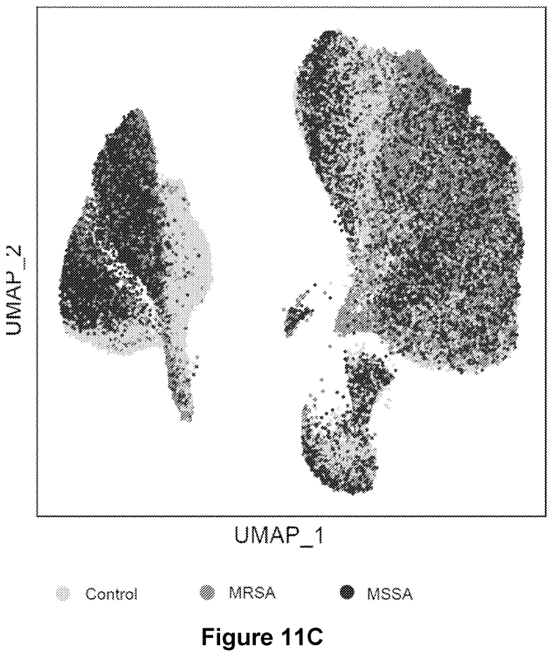

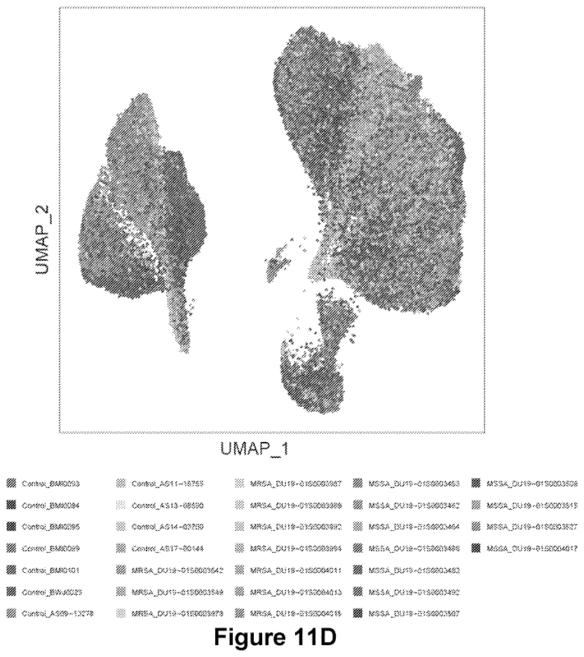

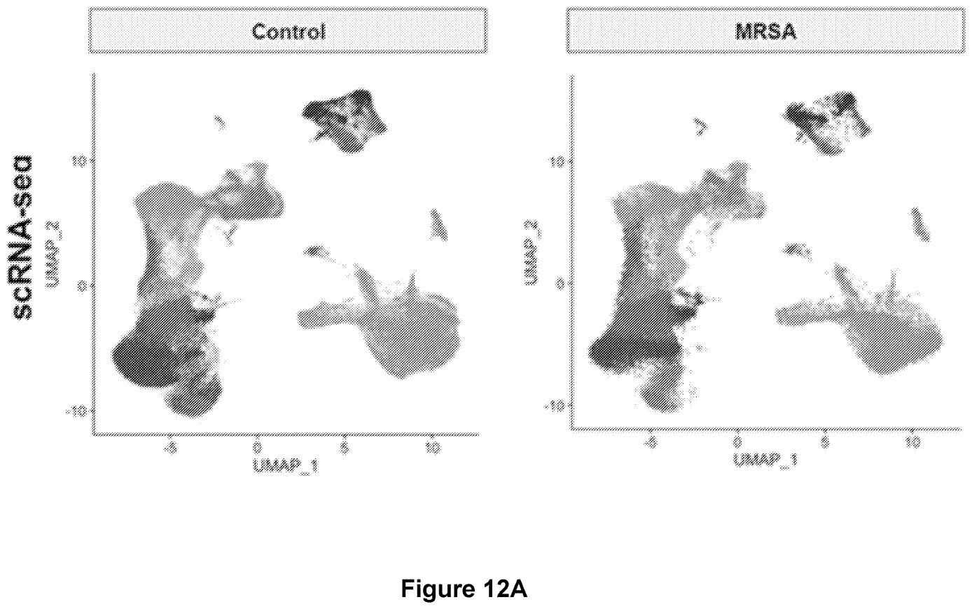

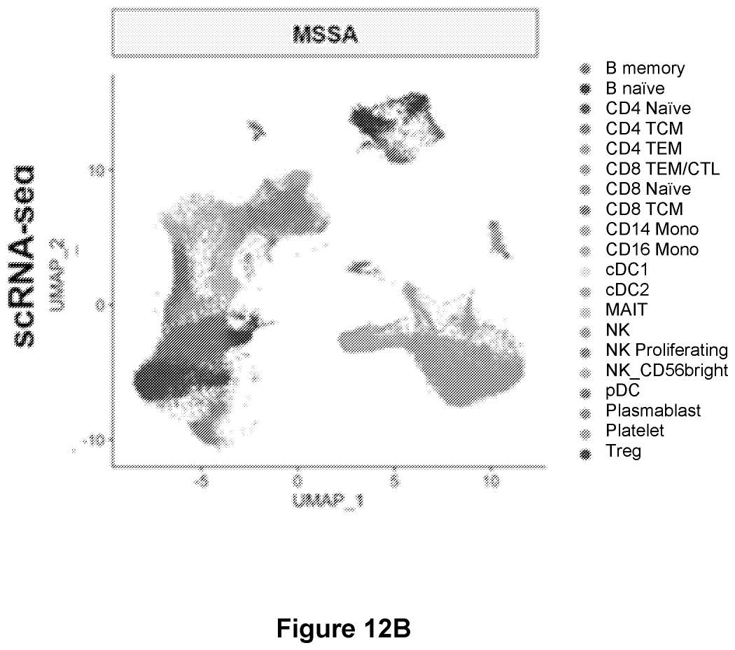

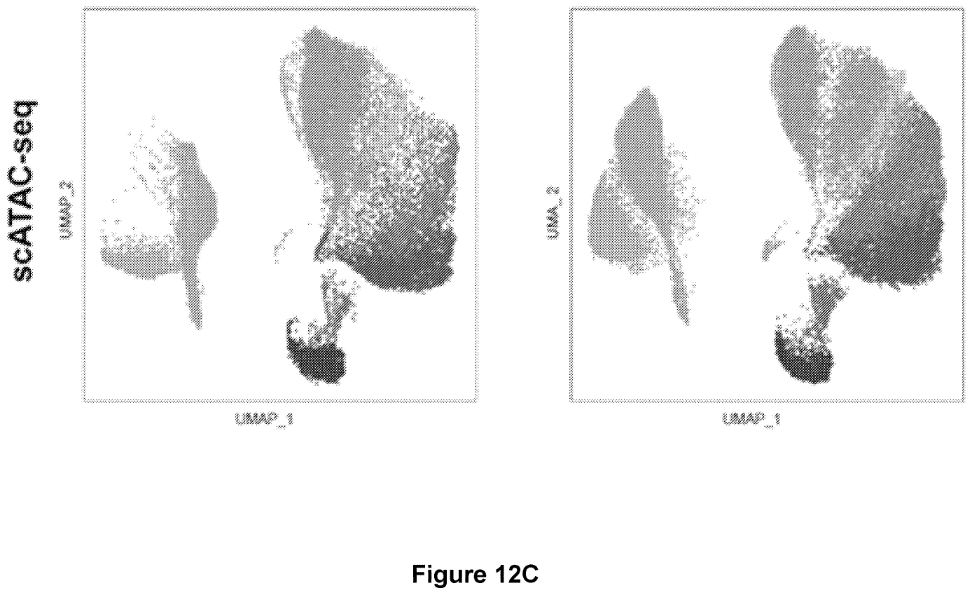

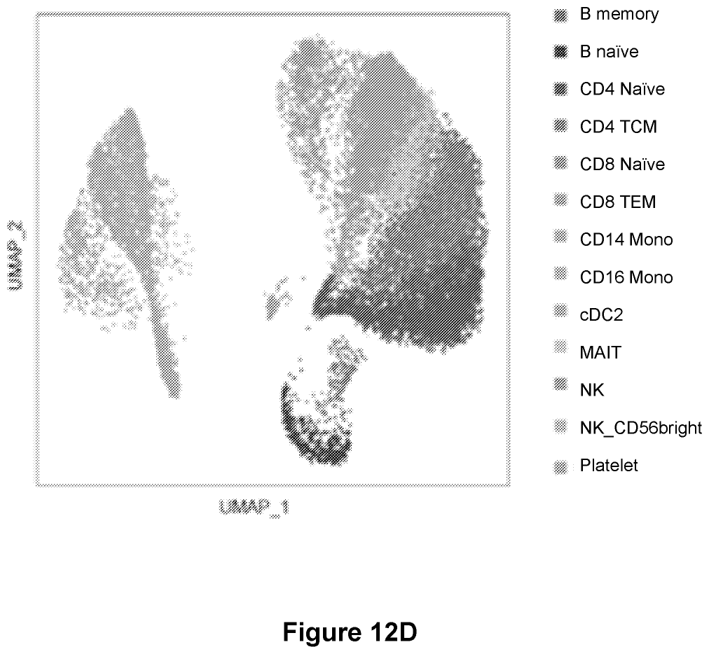

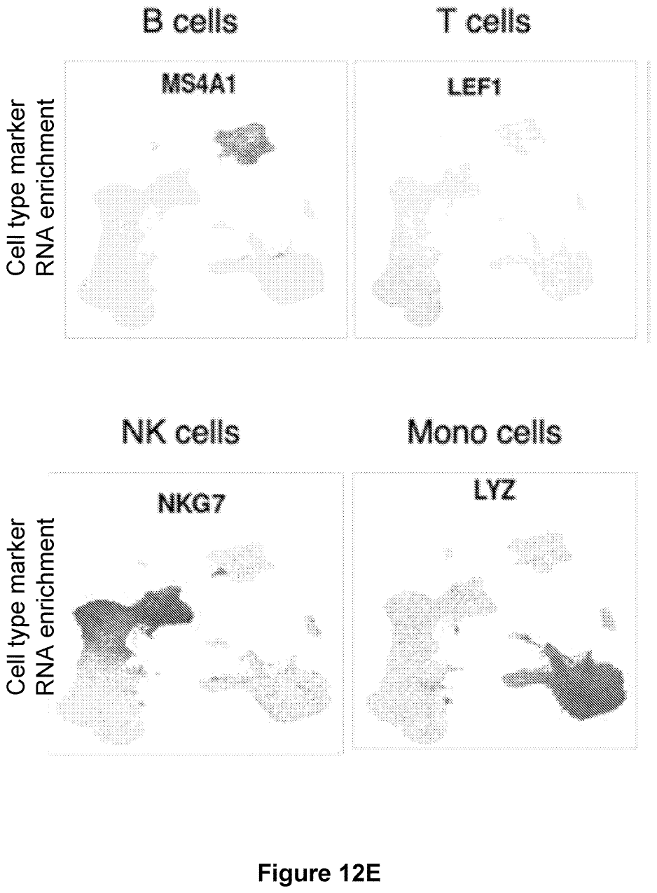

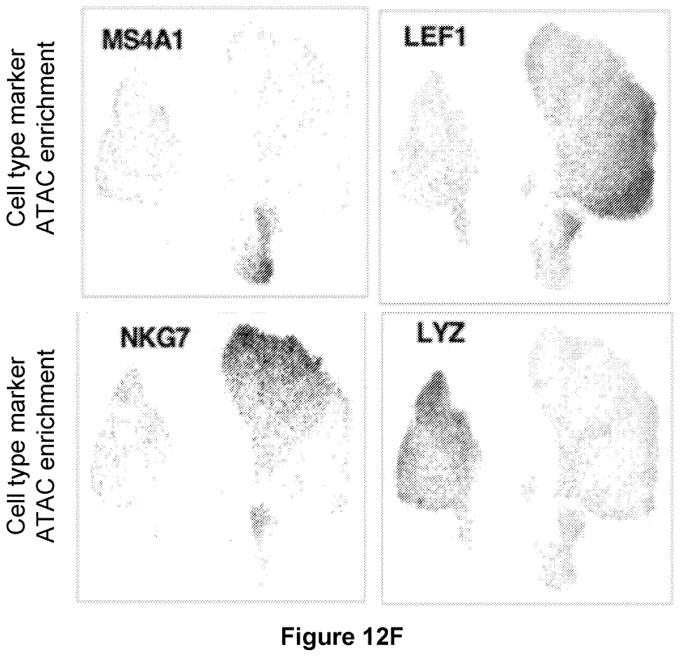

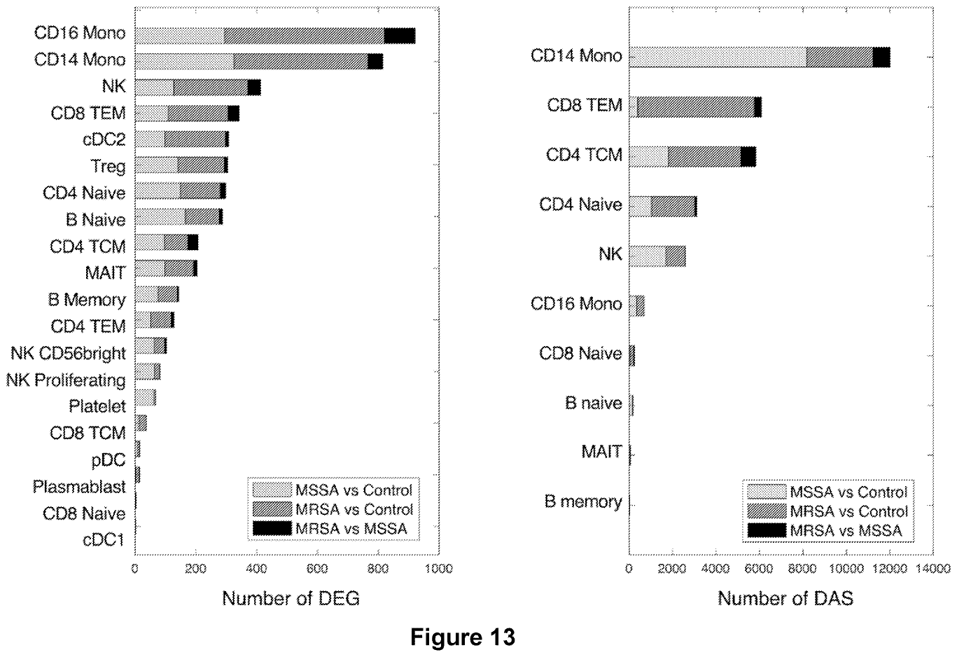

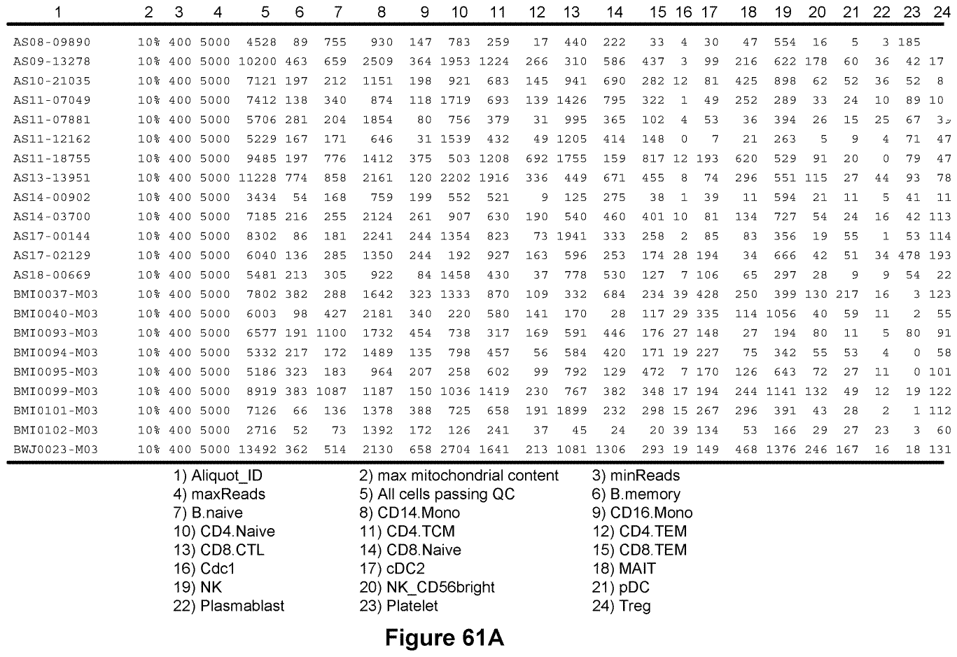

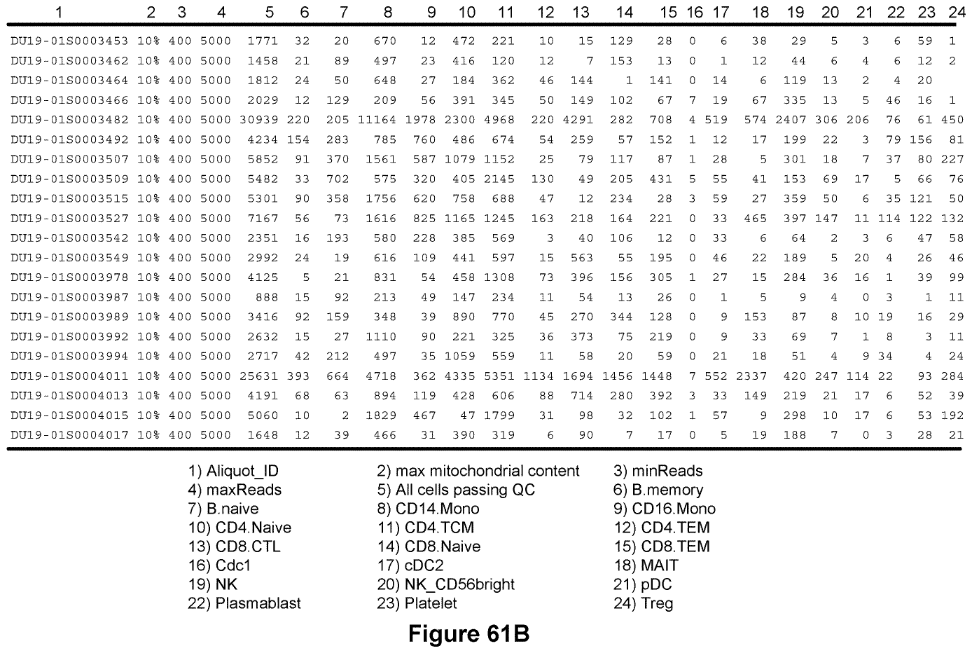

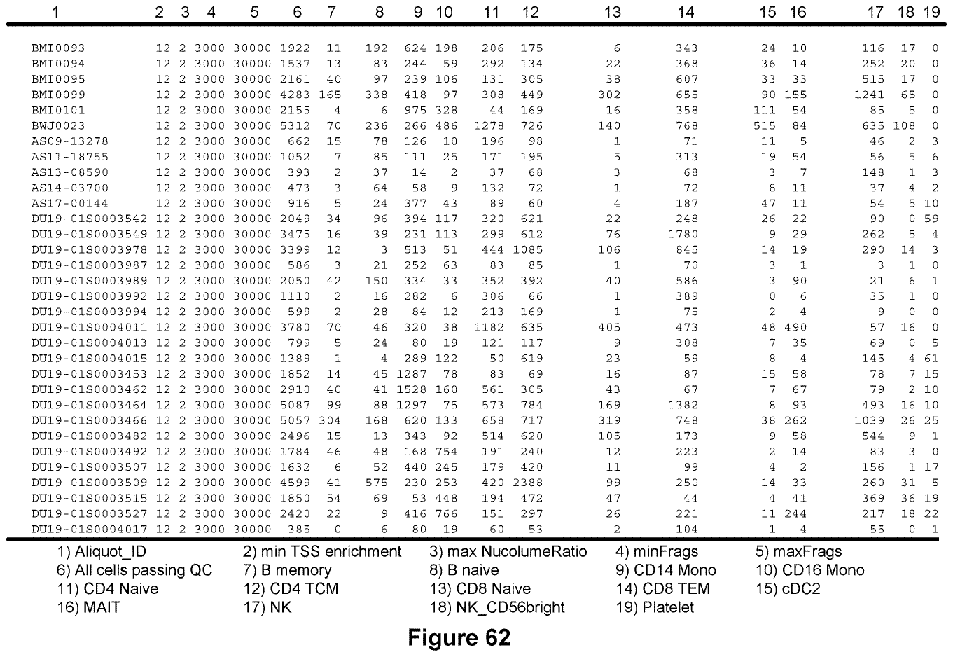

To integrate scRNA-seq data from all samples, a Seurat-based batch correction and cell type annotation pipeline was implemented (See section 1.5.6, below). In total, 276,200 quality cells were selected and labeled (FIG. 5B1; FIGS. 10A-10D; FIGS. 61A and 61B). For scATAC-seq data, the systems and methods of the present disclosure integrated the fragment files from quality samples using ArchR and selected and annotated 70,174 quality cells (FIG. 5C; FIGS. 11A-11D; FIG. 62). In total, 388,860 peaks were identified (FIG. 111B; Table 1.9; Methods: S. aureus scATAC-seq data analysis). Table 1.9 is found at Chen et al., 2023; Supplementary Table 9, which is hereby incorporated by reference in its entirety for all purposes. Thirteen major cell types that surpassed the 200-cell threshold in both scRNA-seq and scATAC-seq data were selected for subsequent analysis (FIGS. 12A-12F). Differential analysis for three contrasts (MRSA vs Control, MSSA vs Control, and MRSA vs MSSA) in each cell type returned a total of 1,477 DEG and 23,434 DAS (FIG. 13; Tables 1.10 and 1.11). Tables 1.10 and 1.11 are found at Chen et al., 2023, “Mapping disease regulatory circuits at cell-type resolution from single-cell multiomics data,” Nature Computational Science 3, pp. 644-657, Supplementary Tables 10 and 11, which is hereby incorporated by reference in its entirety for all purposes.

The systems and methods of the present disclosure identified 1,513 high-confidence regulatory circuits (1,179 sites and 371 genes) within cell types for three contrasts (MRSA vs Control, MSSA vs Control, and MRSA vs MSSA). See Table 1.12 and Section 1.5.11, below. Table 1.12 is found at Chen et al., 2023, “Mapping disease regulatory circuits at cell-type resolution from single-cell multiomics data,” Nature Computational Science 3, pp. 644-657; Supplementary Table 12, which is hereby incorporated by reference in its entirety for all purposes. It has been reported that activation of CD14 monocytes plays a principal role in response to S. aureus infection. See Hao et al., 2021; Skjeflo et al., 2014; Kusunoki et al., 1995, each of which is hereby incorporated by reference in its entirety for all purposes. In the analysis performed by the systems and methods of the present disclosure, CD14 monocytes showed the highest number of regulatory circuits (FIG. 5D). Comparing circuits between cell types the systems and methods of the present disclosure found that these disease-associated circuits are cell type-specific (FIG. 5E). For example, circuits rarely overlapped between very distinct cell types like monocytes and T cells. Between CD14 mono and CD16 mono, or between subtypes of T cells, most circuits are still specific for one cell type. These circuits were further validated using cell type-specific chromatin interactions reported in a reference promoter capture (pc) Hi-C dataset. In all the cell types for which the cell type-specific pcHi-C data was available (B cells, CD4 T cells, CD8 T cells, CD14 monocytes), the circuit peak-gene interactions showed significant enrichment of pcHi-C interactions in matched cell types (FIG. 5F; p-values<0.01, one-side hypergeometric test). For comparison, the systems and methods of the present disclosure also performed the peak-gene interaction enrichment analysis between different cell types, finding significantly lower enrichment levels. These results indicate cell-type specificity of the circuits identified by the systems and method of the present disclosure.

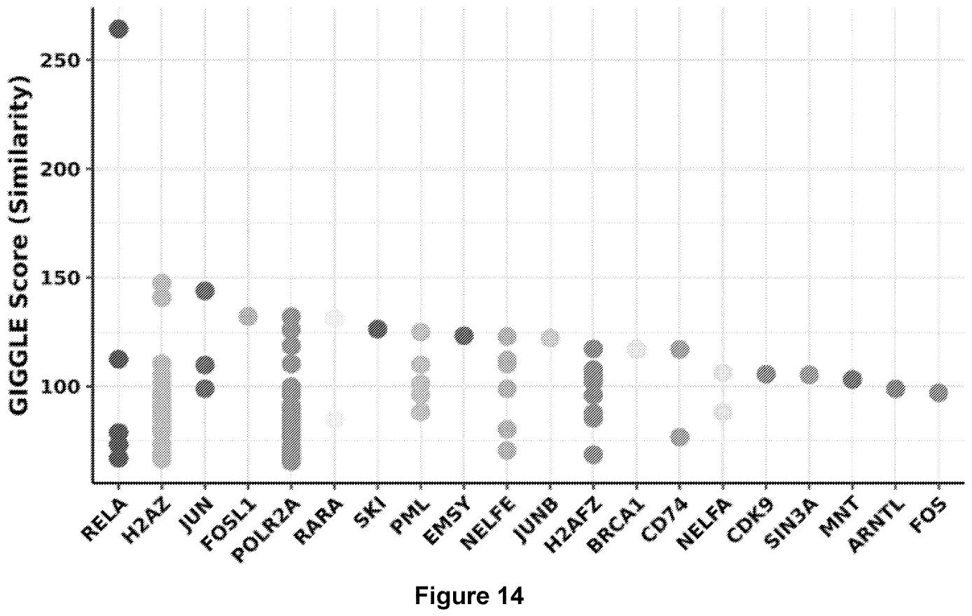

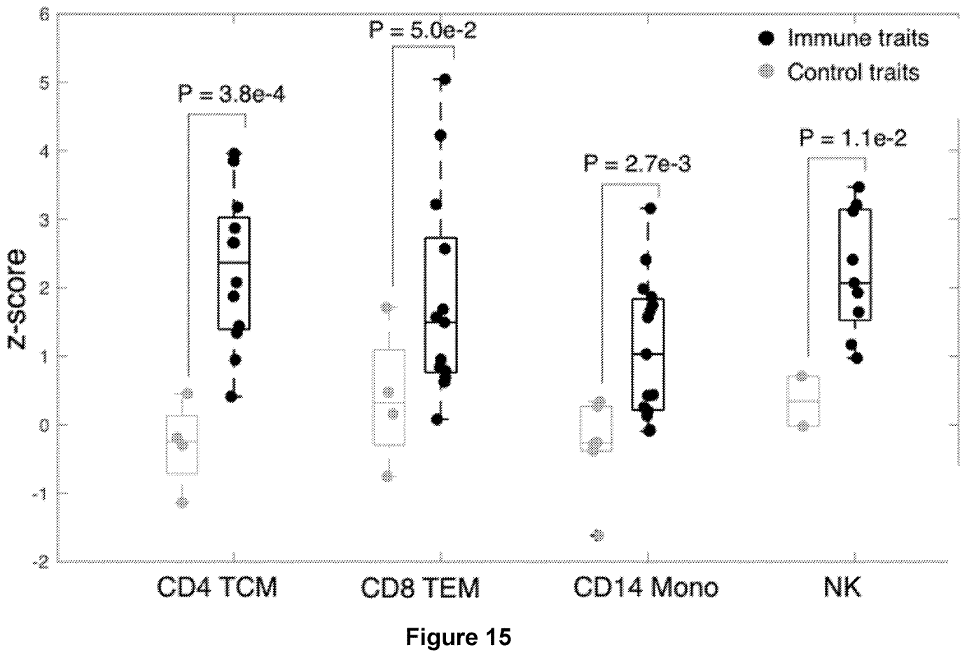

In CD14 monocytes, the systems and methods of the present disclosure identified AP-1 complex proteins as the most important regulators, especially at chromatin sites showing increased activity in infection cells (FIG. 5G). This finding is consistent with the importance of these complexes in gene regulation in response to a variety of infections. See Ludwig et al., 2021; and Gjertsson et al., 2001, each of which is hereby incorporated by reference in its entirety for all purposes. Supporting the accuracy of the identified TFs, the systems and methods of the present disclosure compared circuit chromatin sites with ChIP-seq peaks from the Cistrome database. See Liu et al., 2011, which is hereby incorporated by reference in its entirety for all purposes. The most similar TF ChIP-seq profiles were from AP-1 complex JUN/FOS proteins in blood or bone marrow samples (FIG. 14). Moreover, functional enrichment analysis of the circuit genes showed that cytokine signaling, a known pathway mediated by AP-1 factors and associated with the inflammatory responses in macrophages, was the most enriched (adjusted p-value 2.4e-11, one-side hypergeometric test). See Gillespie et al., 2022; Kyriakis et al., 1999; and Hannemann et al., 2017, each of which is hereby incorporated by reference in its entirety for all purposes.

Regulatory effects of both proximal and distal regions on genes were modeled by the systems and methods of the present disclosure. The chromatin site location was examined relative to the target gene TSS, for circuits chromatin sites and genes identified for CD14 monocytes. Compared to all ATAC peaks called around the circuit genes, a substantially increased proportion of circuit chromatin sites were located 15 Kb to 25 Kb away from the TSS (FIG. 511). This pattern is consistent with the 24 Kb median enhancer distance found by CRISPR-based perturbation in a blood cell line. See Gasperini et al., 2019, which is hereby incorporated by reference in its entirety for all purposes. In addition, nearly 50% of circuit chromatin sites were overlapping with enhancer-like regions in the ENCODE database, further emphasizing that the circuits identified by the systems and methods of the present disclosure are enriched in distal regulatory loci. See Consortium et al., 2020, which is hereby incorporated by reference in its entirety for all purposes. In some embodiments, the systems and methods of the present disclosure also found that these circuit chromatin sites were significantly enriched in inflammatory-associated genomic loci reported in the genome-wide association studies (GWAS) catalog database, suggesting active host epigenetic responses to infectious diseases (FIG. 14; p-value<0.005 when compared to control diseases, two-wide Wilcoxon rank, sum test). Notably, one distal chromatin site (hg38 chr6: 32,484,007-32,484,507) looping to HLA-DRB1 is within the most significant GWAS region (hg38 chr6: 32,431,410-32,576,834) associated with S. aureus infection. See Buniello et al., 2019; DeLorenze et al., 2016, each of which is hereby incorporated by reference in its entirety for all purposes.

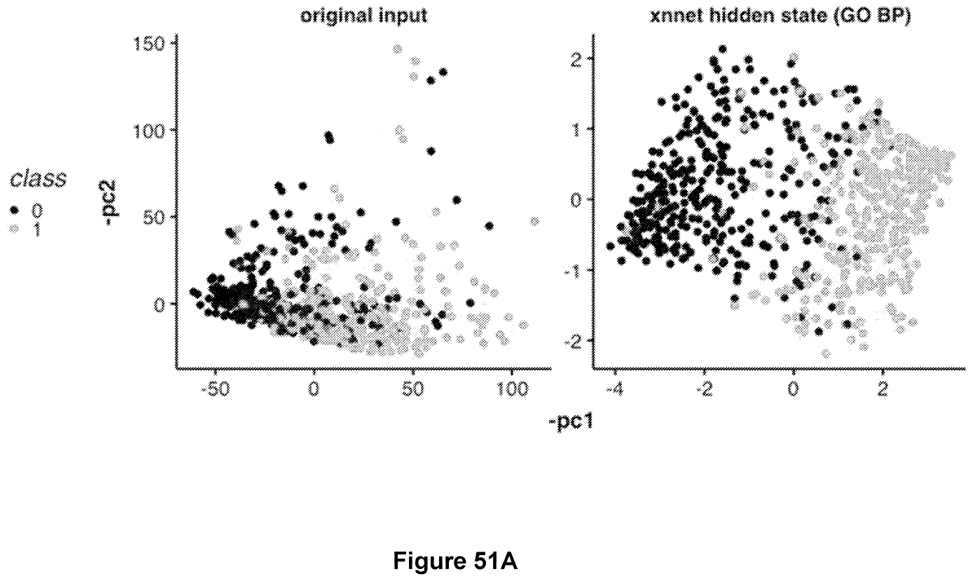

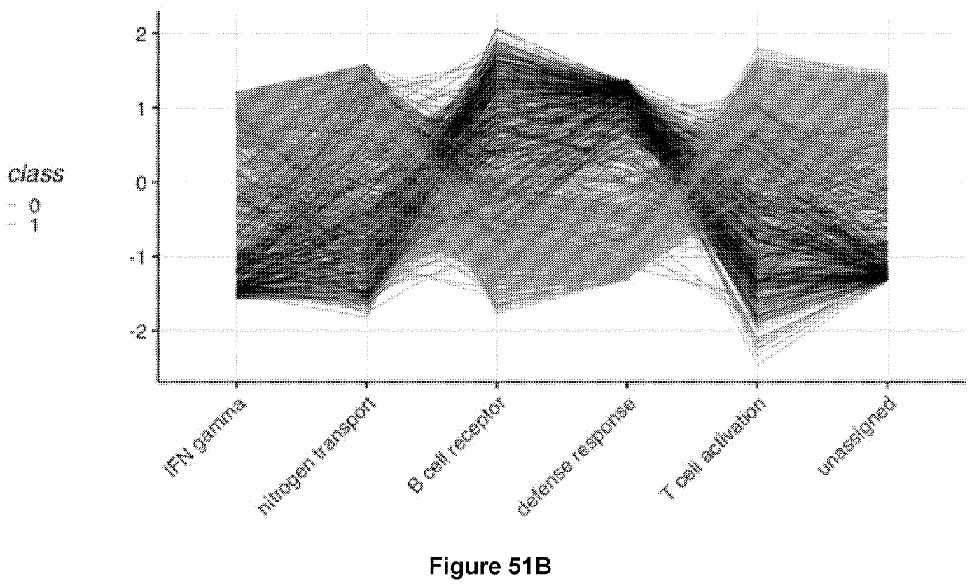

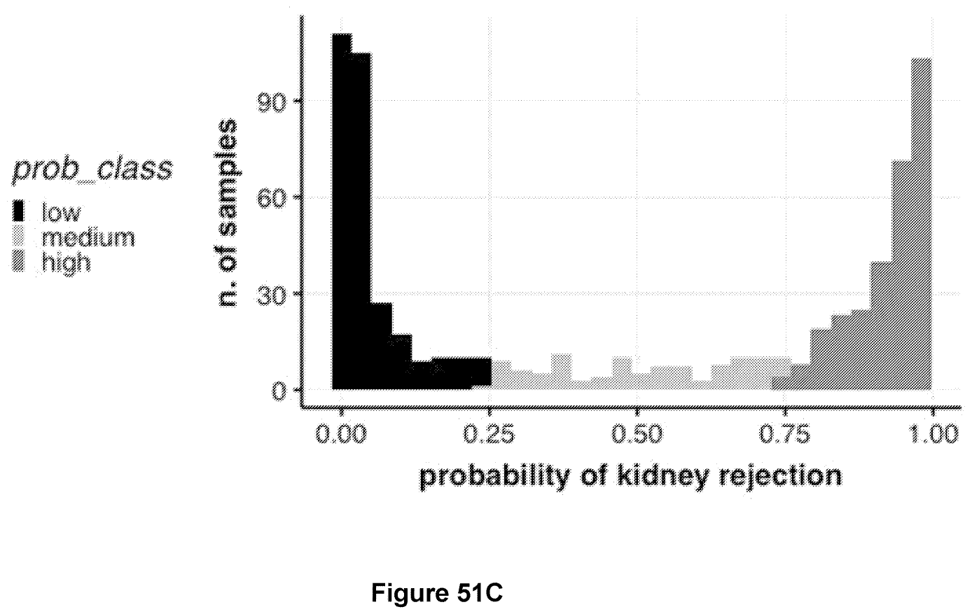

In some embodiments, the systems and methods of the present disclosure compared circuit genes to existing epi-genes whose transcriptions were significantly driven by epigenetic perturbations in CD14 monocytes. See Chen et al., 2016, which is hereby incorporated by reference in its entirety for all purposes. Circuit genes identified by the systems and methods of the present disclosure were significantly enriched with epi-genes (FIG. 51; adjusted p-value<0.005, one-side hypergeometric test) while the remaining DEG not selected by the systems and methods of the present disclosure, or those mappable with DAS either within the same topological domains or closest to each other showed no evidence of being epigenetically driven. These results suggest that the systems and methods of the present disclosure accurately identified regulatory circuits activated in response to S. aureus infection.

1.3.5 S. aureus Infection Prediction

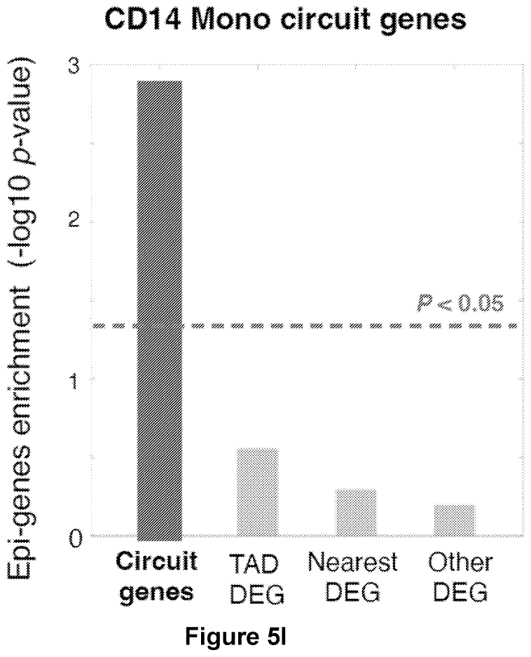

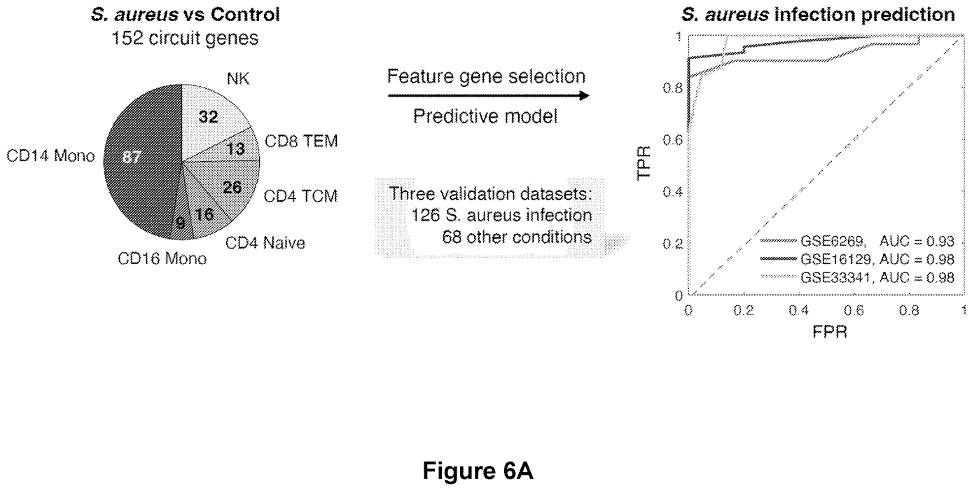

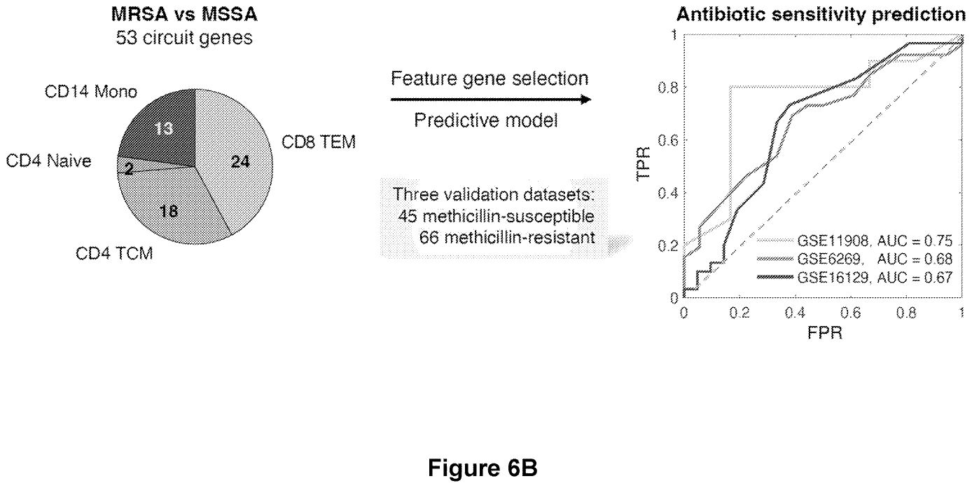

Early diagnosis of S. aureus infection and the strain antibiotic sensitivity is important to appropriate treatment for this life-threatening condition. An evaluation of whether the circuit genes identified by the systems and methods of the present disclosure are in common to MRSA and MSSA could provide a robust signature for predicting the diagnosis of S. aureus infection in general. Within each cell type, the systems and methods of the present disclosure selected circuit genes common to both the MRSA and MSSA analyses, resulting in 152 genes (FIG. 6A; Table 1.12). To evaluate this S. aureus infection, external, public expression data of S. aureus infected subjects was collected. In total, one adult whole-blood and two pediatric PBMC bulk microarray datasets were found that comprised a total of 126 S. aureus infected subjects and 68 uninfected controls. See Ahn et al., 2013; Ramilo et al., 2007; and Ardura et al., 2009, each of which is hereby incorporated by reference in its entirety for all purposes. The use of pediatric validation data has the advantage of providing a much more rigorous test of the robustness of circuit genes identified by the systems and methods of the present disclosure for classifying disease samples in this very different cohort.

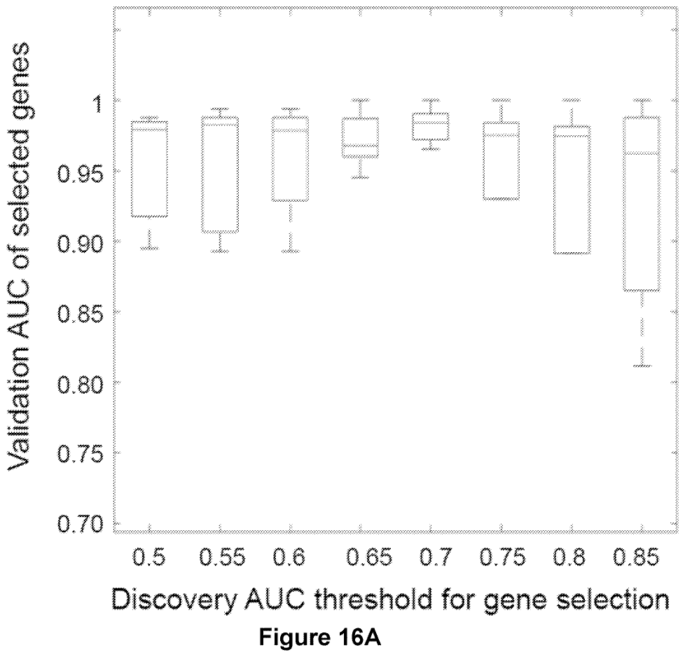

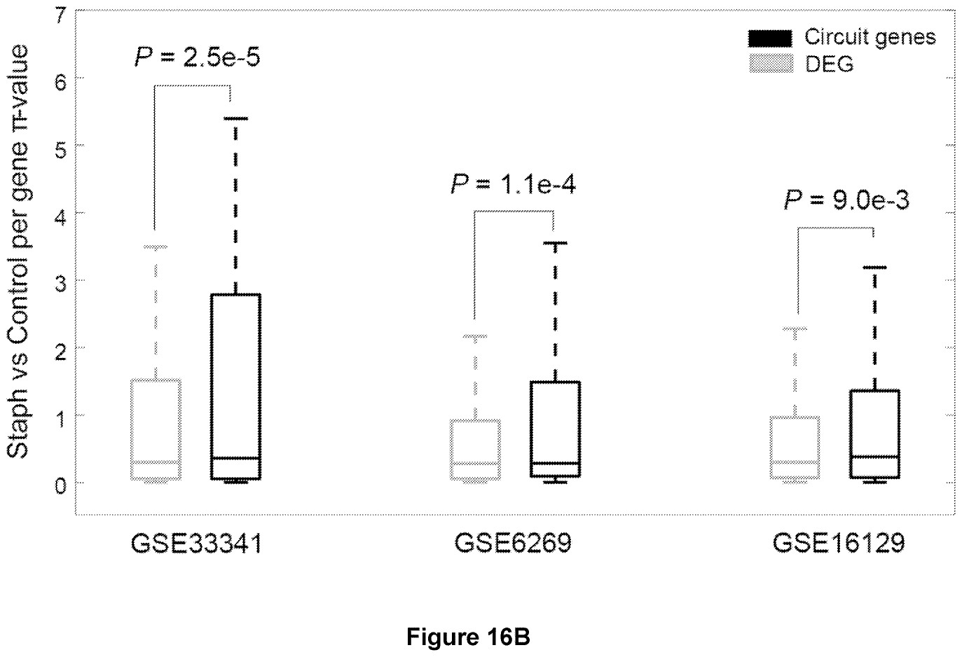

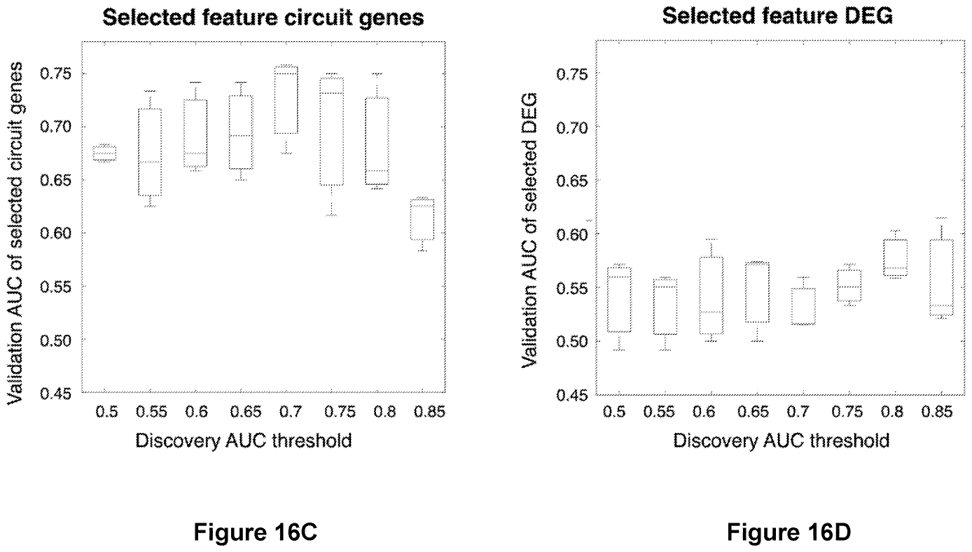

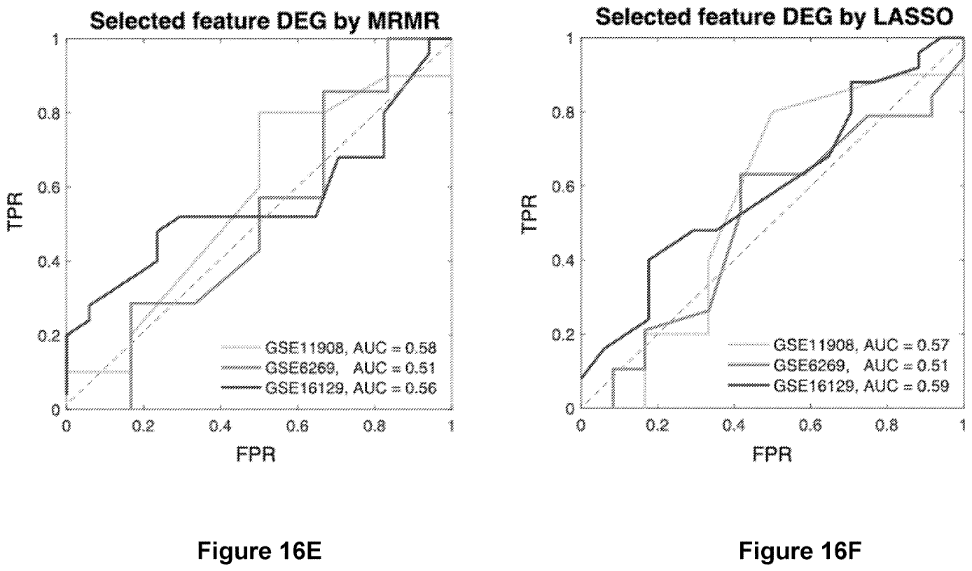

To allow validation using public bulk transcriptome datasets, the systems and methods of the present disclosure refined the 152 circuit genes set by selecting those with robust performance in the dataset at pseudobulk level. An AUROC was calculated for each circuit gene by classifying S. aureus infection and control subjects using pseudo bulk gene expression (aggregated from the discovery scRNA-seq data). One hundred seventeen circuit genes with AUROCs greater than 0.7 were selected (Table 1.13; FIGS. 16A-16F).

TABLE 113
Circuit genes for S.aureus infection prediction

| | | | Discovery | |
|---|---|---|---|---|
| Prediction | Cell type | Circuit Genes | AUC | Selected |
| S.aureus infection | CD14 Mono | SERTAD1 | 0.983 | Yes |
| S.aureus infection | CD14 Mono | PIM1 | 0.963 | Yes |
| S.aureus infection | CD16 Mono | PIM1 | 0.963 | Yes |
| S.aureus infection | CD14 Mono | LUC7L3 | 0.960 | Yes |
| S.aureus infection | CD14 Mono | LDHA | 0.957 | Yes |
| S.aureus infection | CD4 TCM | TUBB4B | 0.946 | Yes |
| S.aureus infection | CD14 Mono | TUBB4B | 0.946 | Yes |
| S.aureus infection | CD14 Mono | ID1 | 0.944 | Yes |
| S.aureus infection | CD4 TCM | SOCS1 | 0.943 | Yes |
| S.aureus infection | CD14 Mono | GADD45B | 0.937 | Yes |
| S.aureus infection | CD14 Mono | RBM6 | 0.937 | Yes |
| S.aureus infection | CD14 Mono | AKIRIN2 | 0.935 | Yes |
| S.aureus infection | CD4 TCM | STMN3 | 0.934 | Yes |
| S.aureus infection | CD14 Mono | MAFB | 0.931 | Yes |
| S.aureus infection | CD14 Mono | UBE2J1 | 0.926 | Yes |
| S.aureus infection | CD4 TCM | TUBA1A | 0.920 | Yes |
| S.aureus infection | CD8 TEM | JUN | 0.919 | Yes |
| S.aureus infection | CD14 Mono | JUN | 0.919 | Yes |
| S.aureus infection | CD8 TEM | TSC22D4 | 0.909 | Yes |
| S.aureus infection | CD4 TCM | HSPA5 | 0.898 | Yes |
| S.aureus infection | CD4 Naive | PNISR | 0.897 | Yes |
| S.aureus infection | CD4 TCM | PNISR | 0.897 | Yes |
| S.aureus infection | CD14 Mono | FCGR1A | 0.894 | Yes |
| S.aureus infection | CD14 Mono | UBALD2 | 0.893 | Yes |
| S.aureus infection | CD4 TCM | UBE2S | 0.887 | Yes |
| S.aureus infection | CD14 Mono | CD300E | 0.877 | Yes |
| S.aureus infection | CD14 Mono | HLA-DMB | 0.877 | Yes |
| S.aureus infection | CD14 Mono | AKNA | 0.874 | Yes |
| S.aureus infection | CD16 Mono | AKNA | 0.874 | Yes |
| S.aureus infection | CD4 TCM | PPP1R15A | 0.871 | Yes |
| S.aureus infection | NK | IFRD1 | 0.870 | Yes |
| S.aureus infection | CD4 Naive | TTC14 | 0.868 | Yes |
| S.aureus infection | CD14 Mono | RBBP4 | 0.867 | Yes |
| S.aureus infection | CD14 Mono | CCR1 | 0.864 | Yes |
| S.aureus infection | CD16 Mono | CCR1 | 0.864 | Yes |
| S.aureus infection | CD14 Mono | HSPA1A | 0.861 | Yes |
| S.aureus infection | CD14 Mono | SIPA1 | 0.855 | Yes |
| S.aureus infection | CD16 Mono | SIPA1 | 0.855 | Yes |
| S.aureus infection | CD14 Mono | CIITA | 0.853 | Yes |
| S.aureus infection | CD14 Mono | PLK3 | 0.851 | Yes |
| S.aureus infection | CD14 Mono | BHLHE40 | 0.848 | Yes |
| S.aureus infection | CD14 Mono | KCNK6 | 0.845 | Yes |
| S.aureus infection | CD8 TEM | ATF4 | 0.842 | Yes |
| S.aureus infection | NK | ATF4 | 0.842 | Yes |
| S.aureus infection | CD4 Naive | FOSB | 0.842 | Yes |
| S.aureus infection | CD4 TCM | FOSB | 0.842 | Yes |
| S.aureus infection | CD8 TEM | FOSB | 0.842 | Yes |
| S.aureus infection | CD14 Mono | MIDN | 0.842 | Yes |
| S.aureus infection | CD14 Mono | CTSA | 0.841 | Yes |
| S.aureus infection | CD14 Mono | IL10RA | 0.839 | Yes |
| S.aureus infection | CD14 Mono | S100A8 | 0.839 | Yes |
| S.aureus infection | CD4 Naive | TNFAIP8 | 0.839 | Yes |
| S.aureus infection | CD4 TCM | TNFAIP8 | 0.839 | Yes |
| S.aureus infection | CD14 Mono | AGFG1 | 0.838 | Yes |
| S.aureus infection | CD14 Mono | ANAPC5 | 0.838 | Yes |
| S.aureus infection | CD14 Mono | PRPF8 | 0.835 | Yes |
| S.aureus infection | CD14 Mono | MAP3K8 | 0.834 | Yes |
| S.aureus infection | NK | MAP3K8 | 0.834 | Yes |
| S.aureus infection | CD4 Naive | CD69 | 0.833 | Yes |
| S.aureus infection | CD4 TCM | CD69 | 0.833 | Yes |
| S.aureus infection | NK | CD69 | 0.833 | Yes |
| S.aureus infection | CD14 Mono | ANKRD13D | 0.832 | Yes |
| S.aureus infection | CD14 Mono | S100A9 | 0.832 | Yes |
| S.aureus infection | CD14 Mono | CDKN2D | 0.829 | Yes |
| S.aureus infection | CD14 Mono | NUP50 | 0.829 | Yes |
| S.aureus infection | CD14 Mono | ZBTB43 | 0.828 | Yes |
| S.aureus infection | CD14 Mono | TPM3 | 0.827 | Yes |
| S.aureus infection | CD4 Naive | GIMAP7 | 0.826 | Yes |
| S.aureus infection | CD4 TCM | GIMAP7 | 0.826 | Yes |
| S.aureus infection | NK | GIMAP7 | 0.826 | Yes |
| S.aureus infection | CD4 TCM | EIF1 | 0.819 | Yes |
| S.aureus infection | CD14 Mono | ID2 | 0.819 | Yes |
| S.aureus infection | NK | IER5 | 0.819 | Yes |
| S.aureus infection | CD14 Mono | TLE3 | 0.819 | Yes |
| S.aureus infection | CD14 Mono | SLC31A2 | 0.818 | Yes |
| S.aureus infection | NK | PDE4D | 0.814 | Yes |
| S.aureus infection | CD14 Mono | CCDC88B | 0.813 | Yes |
| S.aureus infection | CD14 Mono | RNF7 | 0.812 | Yes |
| S.aureus infection | CD8 TEM | ADGRG1 | 0.811 | Yes |
| S.aureus infection | CD14 Mono | LGALS1 | 0.811 | Yes |
| S.aureus infection | NK | LGALS1 | 0.811 | Yes |
| S.aureus infection | CD14 Mono | ARF6 | 0.807 | Yes |
| S.aureus infection | CD14 Mono | KLF6 | 0.807 | Yes |
| S.aureus infection | NK | HDDC2 | 0.805 | Yes |
| S.aureus infection | NK | KLRF1 | 0.805 | Yes |
| S.aureus infection | CD14 Mono | RBP7 | 0.805 | Yes |
| S.aureus infection | CD4 TCM | SBDS | 0.804 | Yes |
| S.aureus infection | CD14 Mono | TNRC6B | 0.801 | Yes |
| S.aureus infection | NK | TNRC6B | 0.801 | Yes |
| S.aureus infection | CD14 Mono | ARHGEF2 | 0.797 | Yes |
| S.aureus infection | CD4 TCM | JUND | 0.794 | Yes |
| S.aureus infection | CD8 TEM | JUND | 0.794 | Yes |
| S.aureus infection | CD14 Mono | ACSL 1 | 0.792 | Yes |
| S.aureus infection | CD14 Mono | SH3BGRL3 | 0.792 | Yes |
| S.aureus infection | NK | EIF4G2 | 0.788 | Yes |
| S.aureus infection | NK | CCDC59 | 0.787 | Yes |
| S.aureus infection | CD14 Mono | SPN | 0.787 | Yes |
| S.aureus infection | NK | CCNH | 0.781 | Yes |
| S.aureus infection | NK | CD44 | 0.771 | Yes |
| S.aureus infection | CD16 Mono | CSNK1G2 | 0.771 | Yes |
| S.aureus infection | NK | IFNG | 0.771 | Yes |
| S.aureus infection | CD4 Naive | EVL | 0.769 | Yes |
| S.aureus infection | CD4 TCM | MCL1 | 0.767 | Yes |
| S.aureus infection | NK | MCL1 | 0.767 | Yes |
| S.aureus infection | CD14 Mono | MARK2 | 0.766 | Yes |
| S.aureus infection | CD16 Mono | APOBEC3G | 0.765 | Yes |
| S.aureus infection | CD14 Mono | S100A12 | 0.765 | Yes |
| S.aureus infection | CD14 Mono | IRF5 | 0.764 | Yes |
| S.aureus infection | CD4 Naive | GIMAP4 | 0.760 | Yes |
| S.aureus infection | CD4 TCM | GIMAP4 | 0.760 | Yes |
| S.aureus infection | CD14 Mono | ARHGAP9 | 0.754 | Yes |
| S.aureus infection | CD14 Mono | ARIDIA | 0.754 | Yes |
| S.aureus infection | CD4 TCM | DNAJA1 | 0.754 | Yes |
| S.aureus infection | NK | DNAJA1 | 0.754 | Yes |
| S.aureus infection | CD14 Mono | STK11 | 0.754 | Yes |
| S.aureus infection | CD14 Mono | PLEKHO1 | 0.751 | Yes |
| S.aureus infection | CD8 TEM | DNAJB6 | 0.749 | Yes |
| S.aureus infection | CD14 Mono | TMEM154 | 0.749 | Yes |
| S.aureus infection | CD14 Mono | RIN3 | 0.748 | Yes |
| S.aureus infection | CD14 Mono | GRK2 | 0.746 | Yes |
| S.aureus infection | CD4 Naive | Clorf56 | 0.744 | Yes |
| S.aureus infection | CD4 TCM | HSP90AA1 | 0.740 | Yes |
| S.aureus infection | NK | HIPK 1 | 0.739 | Yes |
| S.aureus infection | NK | IRF1 | 0.738 | Yes |
| S.aureus infection | CD14 Mono | TNFAIP2 | 0.737 | Yes |
| S.aureus infection | CD4 TCM | TNFAIP3 | 0.737 | Yes |
| S.aureus infection | NK | AMD1 | 0.736 | Yes |
| S.aureus infection | CD14 Mono | ZFP36L2 | 0.736 | Yes |
| S.aureus infection | CD14 Mono | HSPA8 | 0.735 | Yes |
| S.aureus infection | CD14 Mono | PLEK | 0.729 | Yes |
| S.aureus infection | CD8 TEM | RNMT | 0.729 | Yes |
| S.aureus infection | CD4 TCM | ARID5A | 0.725 | Yes |
| S.aureus infection | CD14 Mono | MKNK2 | 0.723 | Yes |
| S.aureus infection | NK | TRA2B | 0.720 | Yes |
| S.aureus infection | CD14 Mono | TIMP2 | 0.719 | Yes |
| S.aureus infection | CD14 Mono | RSRP1 | 0.716 | Yes |
| S.aureus infection | CD14 Mono | PGAM1 | 0.713 | Yes |
| S.aureus infection | NK | SRGN | 0.707 | Yes |
| S.aureus infection | CD14 Mono | CSF3R | 0.703 | Yes |
| S.aureus infection | NK | TSC22D3 | 0.699 | No |
| S.aureus infection | CD4 Naive | HSP90AB1 | 0.698 | No |
| S.aureus infection | CD4 TCM | HSP90AB1 | 0.698 | No |
| S.aureus infection | CD8 TEM | HSP90AB1 | 0.698 | No |
| S.aureus infection | NK | HSP90AB1 | 0.698 | No |
| S.aureus infection | CD4 Naive | UCP2 | 0.697 | No |
| S.aureus infection | CD14 Mono | SP1 | 0.694 | No |
| S.aureus infection | CD14 Mono | TNFRSF1B | 0.687 | No |
| S.aureus infection | CD16 Mono | TNFRSF1B | 0.687 | No |
| S.aureus infection | CD4 TCM | IER2 | 0.686 | No |
| S.aureus infection | CD8 TEM | IER2 | 0.686 | No |
| S.aureus infection | CD4 Naive | FOS | 0.683 | No |
| S.aureus infection | CD4 TCM | FOS | 0.683 | No |
| S.aureus infection | CD8 TEM | FOS | 0.683 | No |
| S.aureus infection | NK | ZNF394 | 0.681 | No |
| S.aureus infection | CD14 Mono | S100A10 | 0.675 | No |
| S.aureus infection | CD14 Mono | NUDT3 | 0.674 | No |
| S.aureus infection | CD14 Mono | AMPD2 | 0.673 | No |
| S.aureus infection | CD16 Mono | AMPD2 | 0.673 | No |
| S.aureus infection | CD14 Mono | RASSF4 | 0.660 | No |
| S.aureus infection | CD8 TEM | RNF125 | 0.660 | No |
| S.aureus infection | CD4 Naive | TCF7 | 0.655 | No |
| S.aureus infection | CD14 Mono | ARL4C | 0.649 | No |
| S.aureus infection | CD14 Mono | CFL1 | 0.643 | No |
| S.aureus infection | CD14 Mono | EFHD2 | 0.642 | No |
| S.aureus infection | CD4 TCM | FMNL1 | 0.639 | No |
| S.aureus infection | CD4 Naive | CDC42SE1 | 0.634 | No |
| S.aureus infection | NK | NR4A2 | 0.632 | No |
| S.aureus infection | CD14 Mono | TMEM50A | 0.624 | No |
| S.aureus infection | CD14 Mono | PRAM1 | 0.619 | No |
| S.aureus infection | CD14 Mono | CD53 | 0.614 | No |
| S.aureus infection | CD14 Mono | ATG16L2 | 0.608 | No |
| S.aureus infection | CD16 Mono | EEF1B2 | 0.607 | No |
| S.aureus infection | CD14 Mono | NOTCH2 | 0.604 | No |
| S.aureus infection | NK | OTULIN | 0.592 | No |
| S.aureus infection | CD4 TCM | PNRC1 | 0.580 | No |
| S.aureus infection | CD14 Mono | PABPC4 | 0.579 | No |
| S.aureus infection | NK | HMGB2 | 0.540 | No |
| S.aureus infection | CD4 Naive | CAP1 | 0.534 | No |
| S.aureus infection | NK | TUBA4A | 0.534 | No |
| S.aureus infection | CD8 TEM | BTG1 | 0.515 | No |
| S.aureus infection | NK | BTG1 | 0.515 | No |
| S.aureus infection | CD4 Naive | ARPC5 | 0.483 | No |
| S.aureus infection | NK | BTG2 | 0.481 | No |

Functional gene enrichment analysis showed that IL-17 signaling was significantly enriched (adjusted p-value 2.4e-4, one-side hypergeometric test), including genes from AP-1, Hsp90, and S100 families. IL-17 had been found to be essential for the host defense against cutaneous S. aureus infection in mouse models. See Cho et al., 2010, which is hereby incorporated by reference in its entirety for all purposes. A SVM model was trained using the selected circuit genes as features and the discovery pseudo bulk gene expression data as input. The trained SVM model was then applied to each of the three validation datasets. The model achieved high prediction performance on all datasets, showing AUROCs from 0.93 to 0.98 (FIG. 6A).

This generalizability of circuit genes for predicting infection in different cohorts suggested that the systems and methods of the present disclosure identifies regulatory processes that are fundamental to the host response to S. aureus sepsis. This was further evaluated by comparing the 117 circuit genes to the 366 filtered DEG (with per gene AUROC>0.7 in the discovery pseudo bulk gene expression data). The differential expression π-value (a statistic score that combines both fold change and p-values) of genes in the validation datasets was examined and significantly higher 71-values were found for the circuit genes (FIG. 16B; p-value 9.0e-3, one-side Wilcoxon rank sum test). See Xiao et al., 2014, which is hereby incorporated by reference in its entirety for all purposes.

1.3.6 S. aureus Antibiotic Sensitivity Prediction

The challenging problem of predicting strain antibiotic sensitivity in S. aureus infection was also addressed. The predictive models trained with DEG for the contrast of MRSA and MSSA on three pediatric PBMC microarray datasets (comprising a total of 66 MRSA and 45 MSSA samples), predictive value was not found (median of prediction AUCs close to 0.5) (FIGS. 16C-16F). See Chaussabel et al., which is hereby incorporated by reference in its entirety for all purposes. And in all tests, the statistical difference between DEG-based prediction scores of the MRSA and MSSA samples in the validation datasets was never significant. These results suggest that using host scRNA-seq data alone fails to identify robust features for predicting the antibiotic sensitivity of the infected strain. These echo previous studies showing that in challenging cases, differential expression analysis using RNA-seq data had limited power to identify robust features for disease-control sample classification. See Wenric et al., 2018, which is hereby incorporated by reference in its entirety for all purposes.

The systems and methods of the present disclosure identified 53 circuit genes from the comparative multiomics data analysis between MRSA and MSSA (Table 1.14).

TABLE 1.14
Circuit genes for S.aureus antibiotic sensitivity prediction

| | | | Discovery | |
|---|---|---|---|---|
| Prediction | Cell type | Circuit Genes | AUC | Selected |
| Antibiotic sensitivity | CD4 TCM | TBCC | 0.927 | Yes |
| Antibiotic sensitivity | CD8 TEM | CALR | 0.909 | Yes |
| Antibiotic sensitivity | CD14 Mono | CALR | 0.909 | Yes |
| Antibiotic sensitivity | CD4 Naive | BRD2 | 0.882 | Yes |
| Antibiotic sensitivity | CD4 TCM | BRD2 | 0.882 | Yes |
| Antibiotic sensitivity | CD8 TEM | TUBB4B | 0.877 | Yes |
| Antibiotic sensitivity | CD4 TCM | JUND | 0.873 | Yes |
| Antibiotic sensitivity | CD4 TCM | ARID5A | 0.855 | Yes |
| Antibiotic sensitivity | CD4 TCM | SRSF7 | 0.845 | Yes |
| Antibiotic sensitivity | CD14 Mono | TUBA1A | 0.836 | Yes |
| Antibiotic sensitivity | CD4 TCM | HNRNPAO | 0.836 | Yes |
| Antibiotic sensitivity | CD14 Mono | IRF1 | 0.832 | Yes |
| Antibiotic sensitivity | CD8 TEM | C16orf54 | 0.823 | Yes |
| Antibiotic sensitivity | CD4 TCM | CORO7 | 0.814 | Yes |
| Antibiotic sensitivity | CD4 TCM | PPP1R15A | 0.809 | Yes |
| Antibiotic sensitivity | CD8 TEM | UBC | 0.805 | Yes |
| Antibiotic sensitivity | CD8 TEM | TGFB1 | 0.805 | Yes |
| Antibiotic sensitivity | CD8 TEM | PPP2R5C | 0.805 | Yes |
| Antibiotic sensitivity | CD4 TCM | NR4A2 | 0.805 | Yes |
| Antibiotic sensitivity | CD14 Mono | NEU1 | 0.805 | Yes |
| Antibiotic sensitivity | CD4 TCM | HNRNPH1 | 0.805 | Yes |
| Antibiotic sensitivity | CD8 TEM | TKT | 0.795 | Yes |
| Antibiotic sensitivity | CD4 TCM | SPOCK2 | 0.791 | Yes |
| Antibiotic sensitivity | CD8 TEM | SPOCK2 | 0.791 | Yes |
| Antibiotic sensitivity | CD8 TEM | PHF1 | 0.773 | Yes |
| Antibiotic sensitivity | CD4 TCM | IDS | 0.773 | Yes |
| Antibiotic sensitivity | CD14 Mono | ALDOA | 0.768 | Yes |
| Antibiotic sensitivity | CD8 TEM | TSC22D3 | 0.759 | Yes |
| Antibiotic sensitivity | CD8 TEM | SURF4 | 0.755 | Yes |
| Antibiotic sensitivity | CD4 TCM | PLK3 | 0.755 | Yes |
| Antibiotic sensitivity | CD8 TEM | PLK3 | 0.755 | Yes |
| Antibiotic sensitivity | CD4 TCM | KDM6B | 0.736 | Yes |
| Antibiotic sensitivity | CD14 Mono | IER3 | 0.736 | Yes |
| Antibiotic sensitivity | CD4 Naive | TNFAIP3 | 0.709 | Yes |
| Antibiotic sensitivity | CD8 TEM | SERPINB1 | 0.705 | Yes |
| Antibiotic sensitivity | CD8 TEM | MAPKAPK2 | 0.705 | Yes |
| Antibiotic sensitivity | CD8 TEM | CDC42SE1 | 0.700 | Yes |
| Antibiotic sensitivity | CD4 TCM | TUBA4A | 0.691 | No |
| Antibiotic sensitivity | CD4 TCM | PTPRC | 0.691 | No |
| Antibiotic sensitivity | CD14 Mono | LSP1 | 0.691 | No |
| Antibiotic sensitivity | CD4 TCM | DUSP2 | 0.686 | No |
| Antibiotic sensitivity | CD8 TEM | PITHD 1 | 0.677 | No |
| Antibiotic sensitivity | CD8 TEM | CCL4 | 0.677 | No |
| Antibiotic sensitivity | CD8 TEM | MPG | 0.673 | No |
| Antibiotic sensitivity | CD8 TEM | ODC1 | 0.668 | No |
| Antibiotic sensitivity | CD14 Mono | HCAR3 | 0.664 | No |
| Antibiotic sensitivity | CD8 TEM | TIMP 1 | 0.655 | No |
| Antibiotic sensitivity | CD4 TCM | MIDN | 0.636 | No |
| Antibiotic sensitivity | CD14 Mono | TUBA1B | 0.632 | No |
| Antibiotic sensitivity | CD14 Mono | RNPEP | 0.627 | No |
| Antibiotic sensitivity | CD14 Mono | KLF4 | 0.623 | No |
| Antibiotic sensitivity | CD8 TEM | PDIA3 | 0.618 | No |
| Antibiotic sensitivity | CD8 TEM | CST7 | 0.614 | No |
| Antibiotic sensitivity | CD14 Mono | STAT2 | 0.609 | No |
| Antibiotic sensitivity | CD14 Mono | NPC2 | 0.577 | No |
| Antibiotic sensitivity | CD8 TEM | FCRL6 | 0.545 | No |
| Antibiotic sensitivity | CD8 TEM | FGFBP2 | 0.532 | No |

A model trained using 32 circuit genes from Table 1.14 that were robustly differential in the discovery pseudobulk data (per gene discovery AUROC>0.7, FIG. 16C) best distinguished antibiotic-resistant and antibiotic-sensitive samples in all three validation datasets, with AUROCs from 0.67 to 0.75 (FIG. 6B1). And the statistical difference between prediction scores of MRSA and MSSA samples was significant (p-value=9.2e-3, two-side Wilcoxon rank sum test). The success of the circuit gene-based model demonstrated that MAGICAL captured generalizable regulatory differences in the host immune response to these closely related bacterial infections.

### 1.4 Discussion

The systems and methods of the present disclosure addressed the previously unmet need of identifying differential regulatory circuits based on single cell multiomics data from different conditions. Importantly, regulatory circuits involving distal chromatin sites were identified. The previously difficult-to-predict distal regulatory regions is increasingly recognized as key for understanding gene regulatory mechanisms. Because the systems and methods of the present disclosure uses DAS and DEG called from a pre-selected cell type, for less distinct cell types or conditions, it is harder to infer circuits at cell type resolution as there are fewer candidate peaks and genes. Also, the systems and methods of the present disclosure analyzes each cell type separately, and cell type specificity is not directly modeled for disease circuit identification. Incorporating an approach to directly identify cell type-specific circuits regulated in disease conditions would be valuable. In some embodiments, the systems and methods of the present disclosure extend the framework to improve circuit identification when cell types are poorly defined and to model cell type specificity.

### 1.5 Methods

### 1.5.1 Human Participants

The COVID-19 study protocol was approved by the Naval Medical Research Center institutional review board (protocol number NMRC.2020.0006) in compliance with all applicable Federal regulations governing the protection of human subjects. The staphylococcus sepsis protocol was reviewed and approved by the Duke Medical School institutional review board (protocol number Pro00102421). Subjects provided written informed consent prior to participation.

### 1.5.2 Statistics & Reproducibility

No statistical methods were used to pre-determine sample sizes. No data were excluded from the analyses. The experiments were not randomized. The Investigators were not blinded to allocation during experiments and outcome assessment.

1.5.3 S. aureus Patient and Control Samples Selection.

Patients with culture-confirmed S. aureus bloodstream infection transferred to DUMC are eligible if pathogen speciation and antibiotic susceptibilities are confirmed by the Duke Clinical Microbiology Laboratory. DNA and RNA samples, PBMCs, clinical data, and the bacterial isolate from the subject are cataloged using an IRB-approved Notification of Decedent Research. In some embodiments, the systems and methods of the present disclosure excluded samples if prior enrollment of the patient in this investigation (to ensure statistical independence of observations) or they are polymicrobial (i.e., more than one organism in blood or urine culture). In total, 21 adult patients were selected with 10 MRSAs and 11 MSSAs. None of them received any antibiotics in the 24 h before the bloodstream infection. Control samples were obtained from uninfected healthy adults matching the sample number and age range of the patient group. In total, 23 samples were collected from two cohorts: 14 controls provided by from the Weill Cornell Medicine, New York, NY, and 9 controls (provided by the Battelle Memorial Institute, Columbus, OH. Meta information of the selected subjects were provided in Table 1.6.

### 1.5.4 PBMC Thawing

Frozen PBMC vials were thawed in a 37° C.-water bath for 1 to 2 minutes and placed on ice. 500 μl of RPMI/20% FBS was added dropwise to the thawed vial, the content was aspirated and added dropwise to 9 ml of RPMI/20% FBS. The tube was gently inverted to mix, before being centrifuged at 300×g for 5 min. After removal of the supernatant, the pellet was resuspended in 1-5 ml of RPMI/10% FBS depending on the size of the pellet. Cell count and viability were assessed with Trypan Blue on a Countess II cell counter (Invitrogen).

1.5.5 S. aureus scRNA-Seq Data Generation

ScRNA-seq was performed as described (10× Genomics, Pleasanton, CA), following the Single Cell 3′ Reagents Kits V3.1 User Guidelines. Cells were filtered, counted on a Countess instrument, and resuspended at a concentration of 1,000 cells/pl. The number of cells loaded on the chip was determined based on the 10× Genomics protocol. The 10× chip (Chromium Single Cell 3′ Chip kit G PN-200177) was loaded to target 5,000-10,000 cells final. Reverse transcription was performed in the emulsion and cDNA was amplified following the Chromium protocol. Quality control and quantification of the amplified cDNA were assessed on a Bioanalyzer (High-Sensitivity DNA Bioanalyzer kit) and the library was constructed. Each library was tagged with a different index for multiplexing (Chromium i7 Multiplex Single Index Plate T Set A, PN-2000240) and quality controlled by Bioanalyzer prior to sequencing.

1.5.6 S. aureus scRNA-Seq Data Analysis

Reads of scRNA-seq experiments were aligned to human reference genome (hg38) using 10× Genomics Cell Ranger software (version 1.2). The filtered feature-by-barcode count matrices were then processed using Seurat. Quality cells were selected as those with more than 400 features (transcripts), fewer than 5,000 features, and less than 10% of mitochondrial content (FIGS. 10A-10D; FIG. 61). Cell cycle phase scores were calculated using the canonical markers for G2M and S phases embedded in the Seurat package. Finally, the effects of mitochondrial reads and cell cycle heterogeneity were regressed out using SCTransform.

To integrate cells from heterogeneous disease samples, the systems and methods of the present disclosure first built a reference by integrating and annotating cells from the uninfected control samples using a Seurat-based pipeline. For batch correction, the systems and methods of the present disclosure identified the intrinsic batch variants and used Seurat to integrate cells together with the inferred batch labels. All control samples were integrated into one harmonized query matrix. Each cell was assigned a cell type label by referring to a reference PBMC single cell dataset. The cell type label of each cell cluster was determined by most cell labels in each. Canonical markers were used to refine the cell type label assignment. This integrated control object was used as reference to map the infected samples.

To avoid artificially removing the biological variance between each infected sample during batch correction, the systems and methods of the present disclosure computationally predicted and manually refined cell types for each sample. All infection samples were projected onto the UMAP of the control object for visualization purpose. In total, 276,200 high-quality cells and 19 cell types with at least 200 cells in each were selected for the subsequent analysis. Within each cell type, differentially expressed genes (DEG) between contrast conditions were first called using the “Findmarkers” function of the Seurat V4 package with default parameters. DEG with Wilcoxon test FDR<0.05, |log 2FC|>0.1 and actively expressed in at least 10% cells (pct>0.1) from either condition were selected. To correct potential bias caused by the different sequencing depth between samples, the systems and methods of the present disclosure ran DEseq256 on the aggregated pseudo bulk gene expression data. Refined DEG passing pseudo bulk differential statistics p-value<0.05 and |log 2FC|>0.3 were selected as the final DEG (Table 1.10).

1.5.7 Nuclei Isolation for scATACseq

Thawed PBMCs were washed with PBS/0.04% BSA. Cells were counted and 100,000-1,000,000 cells were added to a 2 mL-microcentrifuge tube. Cells were centrifuged at 300×g for 5 min at 4° C. The supernatant carefully completely removed, and 0.1× lysis buffer (1×: 10 mM Tris-HCl pH 7.5, 10 mM NaCl, 3 mM MgCl₂, nuclease-free H20, 0.1% v/v NP-40, 0.1% v/v Tween-20, 0.01% v/v digitonin) was added. After a three minute incubation on ice, 1 ml of chilled wash buffer was added. The nuclei were pelted at 500×g for five minutes at 4° C. and resuspended in a chilled diluted nuclei buffer (10× Genomics) for scATAC-seq. Nuclei were counted and the concentration was adjusted to run the assay.

1.5.8 S. aureus scATAC-Seq Data Generation

ScATAC-seq was performed immediately after nuclei isolation and following the Chromium Single Cell ATAC Reagent Kits V1.1 User Guide (10× Genomics, Pleasanton, CA). Transposition was performed in 10 μl at 37° C. for 60 min on at least 1,000 nuclei, before loading of the Chromium Chip H (PN-2000180). Barcoding was performed in the emulsion (12 cycles) following the Chromium protocol. After post GEM cleanup, libraries were prepared following the protocol and were indexed for multiplexing (Chromium i7 Sample Index N, Set A kit PN-3000427). Each library was assessed on a Bioanalyzer (High-Sensitivity DNA Bioanalyzer kit).

1.5.9 S. aureus scATAC-Seq Data Analysis

Reads of scATAC-seq experiments were aligned to human reference genome (hg38) using 10× Genomics Cell Ranger software (version 1.2). The resulting fragment files were processed using ArchR25. Quality cells were selected as those with TSS enrichment>12, the number of fragments>3000 and <30000, and nucleosome ratio<2 (FIG. 11A; FIG. 62). The likelihood of doublet cells was computationally assessed using ArchR's addDoubletScores function and cells were filtered using the ArchR's filterDoublets function with default settings. Cells passing quality and doublet filters from each sample were combined into a linear dimensionality reduction using ArchR's addIterativeLSI function with the input of the tile matrix (read counts in binned 500 bps across the whole genome) with iterations=2 and varFeatures=20000. This dimensionality reduction was then corrected for batch effect using the Harmony method57, via ArchR's addHarmony function. The cells were then clustered based on the batch-corrected dimensions using ArchR's addClusters function. In some embodiments, the systems and methods of the present disclosure annotated scATAC-seq cells using ArchR's addGeneIntegrationMatrix function, referring to a labeled multimodal PBMC single cell dataset. Doublet clusters containing a mixture of many cell types were manually identified and removed. In total, 70,174 high-quality cells and 13 cell types with at least 200 cells in each were selected.

Peaks were called for each cell type using ArchR's addReproduciblePeakSet function with the MACS2 peak caller (FIG. 11B). In total, 388,859 peaks were identified (Table 1.9). Within each cell type, differentially accessible chromatin sites (DAS) between contrast conditions (MRSA vs Control, MSSA vs Control or MRSA vs MSSA) were called from the single cell chromatin accessibility count data using the “getMarkerFeatures” function of ArchR v1.0.225, with parameter settings as testMethod=“wilcoxon”, bias=“log 10(nFrags)”, normBy=“ReadsInPeaks”, and maxCells=15000. Peaks with single cell differential statistics FDR<0.05, |log 2FC|>0.1, and actively accessible in at least 10% cells (pct>0.1) from either condition were selected as DAS. Due to the high false positive rate in single cell-based differential analysis, the systems and methods of the present disclosure further refined the DAS by fitting a linear model to the aggregated and normalized pseudobulk chromatin accessibility data and tested DAS individually about their covariance with sample conditions. Refined DAS passing pseudobulk differential statistics p-value<0.05 and |log 2FC|>0.3 between the contrast conditions were selected as the final DAS (Table 1.11). See Love et al., 2014; Korsunsky et al., 2019; Squair et al., 2021, each of which is hereby incorporated by reference in its entirety for all purposes.

### 1.5.10 MAGICAL

To build candidate regulatory circuits, TFs were mapped to the selected DAS by searching for human TF motifs from the chromVARmotifs library using ArchR's addMotifAnnotations function. See Schep et al., 2017, which is hereby incorporated by reference in its entirety for all purposes. The binding DAS were then linked with DEG by requiring them in the same TAD within boundaries. Then, a candidate circuit is constructed with a chromatin region and a gene in the same domain, with at least one TF motif match in the region.

For each cell type (i.e. i^{th} cell type), MAGICAL (an embodiment of the systems and methods of the present disclosure) inferred the confidence of TF-peak binding and peak-gene looping in each candidate circuit using a hierarchical Bayesian framework with two models: a model of TF-peak binding confidence (B) and hidden TF activity (T) to fit chromatin accessibility (A) for MTFs and P chromatin sites in K_{A,S,}i cells with scATAC-seq measures from S samples; a second model of peak-gene interaction (L) and the refined (noise removed) regulatory region activity (BT) to fit gene expression (R) of G genes in K_{R,S,}i cells with scRNA-seq measures from the same S samples.

$${A}_{P \times {K}_{A , S ,} i} = {B}_{P \times M , i} {T}_{M \times {K}_{A , S ,} i} + {N}_{P \times {K}_{A , S ,} i} , \qquad ( 1 )$$

$${R}_{G \times {K}_{R , S ,} i} = {L}_{G \times P , i} {B}_{P \times M , i} {T}_{M \times {K}_{R , S ,} i} + {N}_{G \times {K}_{R , S ,} i} , \qquad ( 2 )$$

A_{P×K}_{_{A,S,}}ᵢ was a P by K_{A,S,}i matrix with each element a_{p,k}_{_{A,s,}}ᵢ, representing the ATAC read count of p-th chromatin site (ATAC peak) in k_{A,s}-th cell in s-th sample.

R_{G×K}_{_{R,S,}}ᵢ was a G by K_{R,S,}i matrix with each element r_{g,k}_{_{R,s,}}ᵢ representing the RNA read count of g-th gene in k_{R,s}-th cell of s-th sample.

N_{P×K}_{_{A,S,}}ᵢ, and N_{G×K}_{_{R,S,}}ᵢ, represented data noise in corresponding to A_{P×K}_{_{A,S,}}ᵢ and R_{G×K}_{_{R,S,}}ᵢ

B_{P×M,i} was a P by M matrix with each element b_{p,m,i} representing the binding confidence of m-th TF on p-th candidate chromatin site.

L_{G×P,i} was a G by P matrix with each element l_{p,g,i} representing the interaction between p-th chromatin site and g-th gene.

T_{M×K}_{_{R,S,}}ᵢ was a M by K_{A,S,}i matrix with each element t_{m,k}_{_{A,s}}ᵢ representing the hidden TF activity of m-th TF in k_{A,s}-th ATAC cell of s-th sample.

T_{M×K}_{_{R,S,}}ᵢ was a M by K_{T,S}, matrix with each element t_{m,k}_{_{R,s,}}ᵢ representing the hidden TF activity of m-th TF in k_{R,s}-th RNA cell of s-th sample.

T_{M×K}_{_{A,S,}}ᵢ and T_{M×K}_{_{R,S,}}ᵢ were both extended from the same T_{M×s,i} (with elements t_{m,s,i}) by assuming that in i-th cell type and s-th sample, m-th TF's regulatory activities in all ATAC cells and all RNA cells followed an identical distribution of a single variable t_{m,s,i}. Therefore, K_{A,S,i} and K_{R,S,i} can be different numbers and MAGICAL will only estimate the matrix T_{M×s,i}.

To select high-confidence regulatory circuits, MAGICAL estimated the confidence (probability) of TF-peak binding B_{P×M,i} and peak-gene interaction L_{G×P,i} together with the hidden variable T_{M×S,i} in a Bayesian framework.

$$P ( B , T , L \vert A , R ) ∝ P ( R \vert L , B , T ) P ( A \vert B , T ) P ( L ) P ( B ) P ( T ) \qquad ( 3 )$$

Based on the regulatory relationship among chromatin sites, upstream TFs, and downstream genes (as illustrated in FIG. 2), the posterior probability of each variable can be approximated as:

$$P ( T \vert A , B ) ∝ P ( A \vert B , T ) P ( T ) \qquad ( 4 )$$

$$P ( B \vert A , T ) ∝ P ( A \vert B , T ) P ( B ) \qquad ( 5 )$$

$$P ( L \vert R , B , T ) ∝ P ( R \vert L , B , T ) P ( L ) \qquad ( 6 )$$

Although the prior states of b_{p,m,i} and l_{p,g,i} were obtained from the prior information of TF motif-peak mapping and topological domain-based peak-gene pairing, their values were unknown. In some embodiments, the systems and methods of the present disclosure assumed zero-mean Gaussian priors for B, L and the hidden variable T by assuming that positive regulation and negative regulation would have the same priors, which is likely to be true given the fact that there were usually similar numbers of up-regulated and down-regulated peaks and genes after the differential analysis. In some embodiments, the systems and methods of the present disclosure set a high variance (non-informative) in each prior distribution to allow the algorithm to learn the distributions from the input data.

$${b}_{p , m , i} ∼ normal ( {μ}_{B} , {σ}_{B}^{2} ) \qquad ( 7 )$$

$${t}_{m , s , i} ∼ normal ( {μ}_{T} , {σ}_{T}^{2} ) \qquad ( 8 )$$

$${l}_{p , g , i} ∼ normal ( {μ}_{L} , {σ}_{L}^{2} ) \qquad ( 9 )$$

where

$$( {μ}_{B} , {σ}_{B}^{2} ) , ( {μ}_{T} , {σ}_{T}^{2} ) , and ( {μ}_{L} , {σ}_{L}^{2} )$$

are hyperparameters representing the prior mean and variance of TF-peak binding, TF activity, and peak-gene looping variables.

The likelihood functions P(A|B, T) and P(R|L, B, T) represent the fitting performance of the estimated variables to the input data. These two conditional probabilities are equal to the probabilities of the fitting residues N_{P×K}_{_{A,S,}}ᵢ and N_{G×K}_{_{R,S,}}ᵢ, for which the systems and methods of the present disclosure assumed zero-mean Gaussian distributions.

$$A / B , T ∼ normal ( {μ}_{{N}_{A}} , {σ}_{{N}_{a}}^{2} ) , {σ}_{{N}_{a}}^{2} ∼ inversegamma ( {α}_{{N}_{A}} , {β}_{{N}_{A}} ) \qquad ( 10 )$$

$$R / L , B , T ∼ normal ( {μ}_{{N}_{A}} , {σ}_{{N}_{a}}^{2} ) , {σ}_{{N}_{a}}^{2} ∼ inversegamma ( {α}_{{N}_{A}} , {β}_{{N}_{A}} ) \qquad ( 11 )$$

where

$$( {μ}_{{N}_{A}} , {σ}_{{N}_{a}}^{2} ) and ( {μ}_{{N}_{A}} , {σ}_{{N}_{a}}^{2} )$$

are hyperparameters representing the prior mean and variance of data noise in the ATAC and RNA measures. Here, the variance of the signal noise is modelled using inverse Gamma distributions, with hyperparameters (α_{N}_{_{A}}, β_{N}_{_{A}}) and (α_{N}_{_{R}}, β_{N}_{_{R}}) to control the variance of fitting residues (very low probabilities on large variances).

Then, the posterior probability of each variable defined in Eq. (4-6) was still a Gaussian distribution with poster mean \hat{μ} and variance \hat{σ} as shown below:

$${\hat{b}}_{p , m , i} ∼ normal ( {\hat{μ}}_{B , m , i} , {\hat{σ}}_{B , m , i}^{2} ) , \qquad ( 12 )$$

$${\hat{t}}_{m , s , i} ∼ normal ( {\hat{μ}}_{T , m , s , i} , {\hat{σ}}_{T , m , s , i}^{2} ) , \qquad ( 13 )$$

$${\hat{l}}_{p , g , i} ∼ normal ( {\hat{μ}}_{L , i} , {\hat{σ}}_{L , i}^{2} ) . \qquad ( 14 )$$

Gibbs sampling was used to iteratively learn the posterior distribution mean and variance of each set of variables and draw samples of their values accordingly.

For the TF-peak binding events, the posterior mean \hat{μ}_{B,m,i} and variance

$${\hat{σ}}_{B , m , i}^{2}$$

were estimated specifically for m-th TF since the number of binding sites and the positive or negative regulatory effects between TFs could be very different.

$${\hat{μ}}_{B , m , i} = \frac{{\sum }_{s} {\sum }_{k} {t}_{m , s , i} ( {a}_{p , k , s , i} - {\sum }_{{m}^{′}} {b}_{p , {m}^{′} , i} {t}_{{m}^{′} , s , i} ) {σ}_{B}^{2} + {μ}_{B , t} {σ}_{{N}_{A}}^{2}}{{\sum }_{s} {K}_{A , s} {t}_{m , s , i}^{2} {σ}_{B}^{2} + {σ}_{{N}_{A}}^{2}} and \qquad ( 15 )$$

$${\hat{σ}}_{B , m , i}^{2} = \frac{{σ}_{{N}_{A}}^{2} {σ}_{B}^{2}}{{\sum }_{s} {K}_{A , s} {t}_{m , s , i}^{2} {σ}_{B}^{2} + {σ}_{{N}_{A}}^{2}}$$

For TF activities, the posterior mean \hat{μ}_{T,m,s,i} and variance

$${\hat{σ}}_{T , m , s , i}^{2}$$

were estimated specifically for m-th TF and s-th sample using chromatin accessibility data as follows:

$${\hat{μ}}_{T , m , s , i} = \frac{{\sum }_{p} {\sum }_{k} {b}_{p , m} ( {a}_{p , k , s , i} - {\sum }_{{m}^{′}} {b}_{p , {m}^{′}} {t}_{{m}^{′} , s} ) {σ}_{T}^{2} + {μ}_{T} {σ}_{{N}_{A}}^{2}}{{\sum }_{p} {K}_{A , s} {b}_{p , m , i}^{2} {σ}_{T}^{2} + {σ}_{{N}_{A}}^{2}} and \qquad ( 16 )$$

$${\hat{σ}}_{T , m , s , i}^{2} = \frac{{σ}_{{N}_{A}}^{2} {σ}_{T}^{2}}{{\sum }_{p} {K}_{A , s} {b}_{p , m , i}^{2} {σ}_{T}^{2} + {σ}_{{N}_{A}}^{2}}$$

Then, based on the estimated distribution parameters of \hat{μ}_{T,m,s,i} and

$${\hat{σ}}_{T , m , s , i}^{2}$$

of \hat{t}_{m,s,i}, for k_{R,s}-th RNA cell in the same s-th sample the systems and methods of the present disclosure draw a TF regulatory activity sample as \hat{t}_{m,k}_{_{R}}_{,s,i}. For p-th peak, the systems and methods of the present disclosure were able to reconstruct its chromatin activity in the RNA cell as â_{p,k}_{_{R}}_{s,i}=Σₘ\hat{b}_{p,m,i}\hat{t}_{m,k}_{_{R}}_{,s,i}, and for g-th gene, the systems and methods of the present disclosure further estimated the interaction confidence \hat{l}_{p,g,i} between p-th peak and g-th gene. The peak-gene interaction distribution parameters \hat{μ}_{L,i} and

$${\hat{σ}}_{L , i}^{2}$$

were estimated as follows:

$${\hat{μ}}_{L , i} = \frac{{\sum }_{s} {\sum }_{k} {\hat{a}}_{p , {k}_{R} , s , i} ( {r}_{g , k , s , i} - {\sum }_{{p}^{′}} {l}_{g , {p}^{′}} {\hat{a}}_{{p}^{′} , {k}_{R} , s , i} ) {σ}_{L}^{2} + {μ}_{L} {σ}_{{N}_{R}}^{2}}{{\sum }_{s} {{K}_{{k}_{R , s}} ( {\hat{a}}_{p , {k}_{R} , s , i} )}^{2} {σ}_{L}^{2} + {σ}_{{N}_{A}}^{2}} and \qquad ( 17 )$$

$${\hat{σ}}_{L}^{2} = \frac{{σ}_{{N}_{R}}^{2} {σ}_{L}^{2}}{{\sum }_{s} {{K}_{{k}_{R , s}} ( {\hat{a}}_{p , {k}_{R} , s , i} )}^{2} {σ}_{L}^{2} + {σ}_{{N}_{R}}^{2}}$$

In n-th round of Gibbs estimation, after learning all distributions, the systems and methods of the present disclosure estimated the confidence of each linkage by linearly mapping the sampled values of \hat{b}_{p,m,i} and \hat{l}_{p,g,i} in the range of (−∞,∞) to probabilities in (0,1) as follows:

$$P ( state ( {b}_{p , m , i} \vert n ) = 1 ) = \frac{exp \{ ( {\hat{b}}_{p , m , i} - {\hat{μ}}_{B , m , i} ) / 2 {σ}_{B , m , i}^{2} \}}{exp \{ ( {\hat{b}}_{p , m , i} - {\hat{μ}}_{B , m , i} ) / 2 {σ}_{B , m , i}^{2} \} + exp \{ ( 0 - {\hat{μ}}_{B , m , i} ) / 2 {σ}_{B , m , i}^{2} \}} . \qquad ( 18 )$$

$$P ( state ( {l}_{p , g , i} \vert n ) = 1 ) = \frac{exp \{ ( {\hat{l}}_{p , g , i} - {\hat{μ}}_{L , i} ) / 2 {σ}_{L , i}^{2} \}}{exp \{ ( {\hat{l}}_{p , g , i} - {\hat{μ}}_{L , i} ) / 2 {σ}_{L , i}^{2} \} + exp \{ ( 0 - {\hat{μ}}_{L , i} ) / 2 {σ}_{L , i}^{2} \}} . \qquad ( 19 )$$

Binary state samples were then drawn based on the confidence of each linkage and were then used to initiate the next round of estimations. After running a long sampling process (in total N rounds) and accumulating enough samples on the binary states of TF-peak bindings and peak-gene interactions, the systems and methods of the present disclosure calculated the sampling frequency of each linkage as a posterior probability.

$$\{ \begin{matrix}P ( state ( {b}_{p , m , i} ) = 1 ) = \frac{{\sum }_{n} state ( {b}_{p , m , i} \vert n )}{N} \\ P ( state ( {l}_{p , g , i} ) = 1 ) = \frac{{\sum }_{n} state ( {l}_{p , g , i} \vert n )}{N}\end{matrix} \qquad ( 20 )$$

### 1.5.11 MAGICAL Analysis of S. Aureus Single-Cell Multiomics Data

For each cell type, given DAS and DEG of contrast conditions (MRSA vs Control, MSSA vs Control or MRSA vs MSSA), MAGICAL was first initialized by mapping prior TF motifs from the ‘chromVARmotifs’ library to DAS using ArchR's addMotifAnnotations. Because there is no PBMC cell type Hi-C data publicly available, the systems and methods of the present disclosure are using TAD boundaries from a lymphoblastoid cell line, GM12878, which was originally generated by EBV transformation of PBMCs. The TAD boundary structure is closely conserved between the lymphoblastoid cell lines and primary PBMC and between cell types. See Anderson et al., 1984; Tan et al., 2018; McArthur et al., 2021, each of which is hereby incorporated by reference in its entirety for all purposes. In some embodiments, the systems and methods of the present disclosure called TAD boundaries from a GM12878 cell line Hi-C profile using TopDom. See Rao et al., 2014; Shin et al., 2016, each of which is hereby incorporated by reference in its entirety for all purposes. About 6000 topological domains were identified. For each contrast, the systems and methods of the present disclosure built candidate circuits by pairing DAS with TF binding sites with DEG in the same domain. MAGICAL was run 10000 times to ensure that the sampling process converged to stable states. This process was repeated for all cell types and the top 10% high confidence circuit predictions were selected from each cell type for validation analysis.

### 1.5.12 MAGICAL Analysis of COVID-19 Single-Cell Multiomics Data

As a proof of concept for contrast condition single cell multiomics data analysis, MAGICAL was applied to a public PBMC COVID-19 single-cell multiomics dataset5 with samples collected from patients with different severity and heathy controls. For each of the three selected cell subtypes (CD8 TEM, CD14 Mono, and NK), from the original publication the systems and methods of the present disclosure downloaded DEG for two contrasts: mild vs control and severe vs control. For each of the selected cell types, DAS were called respectively for mild vs control and severe vs control using ArchR's functions and thresholds as introduced in the paper. MAGICAL was initialized by mapping prior TF motifs from the ‘chromVARmotifs’ library to DAS using ArchR's addMotifAnnotations. As explained above, the systems and methods of the present disclosure used TAD boundary information of ~6000 domains identified in GM12878 cell line as prior. Then, DAS with TF binding sites were paired with DEG in the same TAD and the initial candidate regulatory circuits were constructed. Respectively for mild and severe COVID-19, MAGICAL was run 10000 times to ensure that the sampling process converged to stable states. This process was repeated for all selected cell types. The chromatin sites and genes in the top 10% predicted high confidence circuits in each cell type were selected as disease associated.

1.5.13 COVID-19 PBMC Samples of Validation scATAC-Seq Data

To validate chromatin sites associated with mild COVID-19, PBMC samples were obtained from the COVID-19 Health Action Response for Marines (CHARM) cohort study, which has been previously described. See Letizia et al., 2021, which is hereby incorporated by reference in its entirety for all purposes. The cohort is composed of Marine recruits that arrived at Marine Corps Recruit Depot-Parris Island (MCRDPI) for basic training between May and November 2020, after undergoing two quarantine periods (first a home-quarantine, and next a supervised quarantine starting at enrolment in the CHARM study) to reduce the possibility of SARS-CoV-2 infection at arrival. Participants were regularly screened for SARS-CoV-2 infection during basic training by PCR, serum samples were obtained using serum separator tubes (SST) at all visits, and a follow-up symptom questionnaire was administered. At selected visits, blood was collected in BD Vacutainer CPT Tube with Sodium Heparin and PBMC were isolated following the manufacturer's recommendations. PBMC samples from six participants (five males and one female) who had a COVID-19 PCR positive test and had mild symptoms (sampled 3-11 days after the first PCR positive test), and from three control participants (three males) that had a PCR negative test at the time of sample collection and were seronegative for SARS-CoV-2 IgG were used. New scATAC-seq data were generated following the same protocol as described above (Table 1.2).

1.5.14 COVID-19 PBMC scATACseq Data Analysis

Reads of scATAC-seq experiments were aligned to human reference genome (hg38) using 10× Genomics Cell Ranger software (version 1.2). The resulting fragment files were processed using ArchR. Quality cells were selected as those with TSS enrichment>12, the number of fragments>3000 and <30000, and nucleosome ratio<2. The likelihood of doublet cells was computationally assessed using ArchR's addDoubletScores function and cells were filtered using the ArchR's filterDoublets function with default settings. A total of 15,836 high quality cells in the infection group and 9,125 cells in the control group were selected after QC analysis (FIGS. 9A-9E). These cells were combined into a linear dimensionality reduction using ArchR's addIterativeLSI function with the input of the tile matrix (read counts in binned 500 bps across the whole genome) with iterations=2 and varFeatures=20000. The cells were then clustered using ArchR's addClusters function. scATAC-seq cells were annotated using ArchR's addGeneIntegrationMatrix function, referring to a labeled multimodal PBMC single cell dataset. Doublet clusters containing a mixture of many cell types were manually identified and removed.

Peaks were called for each cell type using ArchR's addReproduciblePeakSet function with peak caller MACS226 (FIGS. 9A-9D). In total, 284,525 peaks were identified (Table 1.4). For each of the three selected cell types (CD8 TEM, CD14 Mono and NK), chromatin sites with single cell differential statistics FDR<0.05 and |log 2FC|>0.1 between COVID-19 and control conditions and actively accessible in at least 10% cells (pct>0.1) from either condition were selected. Refined peaks passing pseudobulk differential statistics p-value<0.05 and |log 2FC|>0.3 between the contrast conditions were finally selected as the validation peak set (Table 1.5).

### 1.5.15 COVID-19 Circuit Peaks and Genes Accuracy Evaluation

The number of peaks/genes reported by each COVID-19 study would be different due to the difference in the number of recruited patients and collected cells. To overcome the issue caused by the imbalanced number between discovery and validation dataset or between differential peaks/genes and circuit sites/genes in comparison, in each comparison, the larger peak/gene set was randomly down sampled to match the smaller number of peaks/genes in the other set. The precision (site reproduction rate) is calculated to assess the accuracy of each peak/gene set.

### 1.5.16 MAGICAL Analysis of 10×PBMC Single-Cell True Multiome Data

For benchmarking, MAGICAL was applied to a 10×PBMC single cell multiome dataset including 108,377 ATAC peaks, 36,601 genes, and 11,909 cells from 14 cell types. MAGICAL used the same candidate peaks and genes as selected by TRIPOD for fair performance comparison. Two different priors were used to pair candidate peaks and genes: (1) the peaks and genes were within the same TAD from the GM12878 cell line; (2) the centers of peaks and the TSS of genes were within 500K bps. MAGICAL inferred regulatory circuits with each prior and used the top 10% predictions for accuracy assessment. High confidence peak-gene interactions predicted by TRIPOD on the same data were directly downloaded from the supplementary tables of their publication. Two baseline approaches of peak-gene pairing were included: pairing all peaks with each gene if they are in the same TAD or pairing only the nearest peak to gene based on their genomic distance. To fairly assess the accuracy of MAGICAL weighted peak-gene interactions and the results (paired or non-paired) from TRIPOD or baseline approaches, the systems and methods of the present disclosure selected the top 10% predictions by MAGICAL as the final peak-gene pairing. These pairs were overlapped with the curated 3D genome interactions in blood context from the 4DGenome database and calculated the precision for each approach.

### 1.5.17 MAGICAL Analysis of GM12878 Cell Line SHARE-Seq Data

For benchmarking, MAGICAL was also applied to a GM12878 cell line SHARE-seq dataset. For fair comparison, MAGICAL used the same candidate peaks and genes as selected by FigR. MAGICAL was initialized with two different priors to pair candidate peaks and genes: (1) the peaks and genes were within the same prior TAD from the GM12878 cell line; (2) the centers of peaks and the TSS of genes were within 500 k bps. MAGICAL inferred regulatory circuits under each setting and used the top 10% predictions for accuracy assessment. High confidence peak-gene interactions predicted by FigR were directly downloaded from the supplementary tables of the original publication. Similarly, the top 10% predictions by MAGICAL and interactions paired by the two baseline approaches mentioned above were selected. Peak-gene interactions predicted by each approach were overlapped with GM12878 H3K27ac HiChIP chromatin interactions for precision evaluation.

### 1.5.18 Validating Predicted Peak-Gene Interactions

To assess the precision of the predicted circuit peak-gene interactions, the systems and methods of the present disclosure assumed a corrected inferred peak-gene pair should be also connected by a chromatin interaction reported by Hi-C or similar experiments. To check this, each peak was extended to 2 kb long and then checked for overlapping with one end of a physical chromatin interaction. For genes, the systems and methods of the present disclosure checked if the gene promoter (−2 kb to 500b of TSS) overlapped the other end of the interaction. Precision was calculated as the proportion of overlapped chromatin interactions among the predicted peak-gene interactions. The significance of enrichment of overlapped chromatin interactions was assessed using hypergeometric p-value, with all candidate peak-gene pairs as background.

### 1.5.19 GWAS Enrichment Analysis

To assess the enrichment of GWAS loci of inflammatory diseases in circuit chromatin sites in each cell type, significant GWAS loci were downloaded from GWAS catalog for inflammatory diseases and control diseases. GREGOR was used to assess the enrichment of GWAS loci at which either the index SNP or at least one of its LD proxies overlaps with a circuit chromatin site, using pre-calculated LD data from 1000G EUR samples. See Chen et al., 2023, which is hereby incorporated by reference in its entirety for all purposes. The enrichment p-value of each disease GWAS was converted to a z-score. With each cell type, enrichment scores for traits with fewer than 5 overlapped GWAS SNPs with circuit sites were hold out. Also, as all reference data used by GREGOR is hg19 based, genome coordinates of testing regions were mapped from hg38 to hg19.

1.5.20 Predicting S. aureus Infection State

To refine circuit genes lately used for predicting infection diagnosis in microarray gene expression data, the capability of each circuit gene on distinguishing infection and control samples, or MRSA and MSSA samples, was assessed using sample level pseudobulk gene expression data, aggregated from the discovery scRNA-seq datasets. The total number of reads of each sample was normalized to 1e7. The normalized RNA read counts across all samples were log and z-score transformed. For each circuit gene, a discovery AUROC (area under the ROC curve) was calculated by comparing the scRNA-seq gene expression-based sample ranking against the contrasted sample groups. Circuit genes were prioritized based on AUROCs. An SVM model was trained using the top-ranked circuit genes as features and their normalized pseudobulk expression data as input. The model was then tested on independent microarray datasets. The microarray gene expression data was also log and z-score transformed to ensure a similar distribution to the training data. For comparison, top DEG prioritized by discovery AUROC or by other approaches like the Minimum Redundancy Maximum Relevance (MRMR) algorithm or LASSO regression were also tested on the same microarray datasets.

### 1.6 Data Availability

The 10×PBMC single cell multiome dataset can be downloaded from support.10xgenomics.com/single-cell-multiome-atac-gex/datasets/1.0.0/pbmc_granulocyte_sorted_10k. Users will need to provide their contact information to access the download webpage where the filtered feature barcode matrix (HDF5 format) can be downloaded. The reference multimodal PBMC single cell dataset (H5 Seurat data file) can be downloaded from atlas.fredhutch.org/nygc/multimodal-pbmc/. The GWAS catalog database can be accessed at ebi.ac.uk/gwas/docs/file-downloads. SNPs associated with each disease used in this paper can be extracted from the downloadable file “All associations v1.0”. Home sapiens chromatin interactions data can be downloaded from 4dgenome.research.chop.edu/Download.html. Home sapiens transcription factor ChIP-seq profiles can be downloaded at cistrome.org/db/. Users can also provide their customized peaks in BED format to the server dbtoolkit.cistrome.org/and identify transcription factors that have a significant binding overlap. Home sapiens candidate enhancers annotated by ENCODE can be downloaded at screen.encodeproject.org/. The chromVARmotifs library is available at github.com/GreenleafLab/chromVARmotifs. The source single cell data collected in this study is publicly accessible at the GEO repository www.ncbi.nlm.nih.gov/geo/, accession no. GSE220190) and the Zenodo repository.

### 1.7 Code Availability

The source code of MAGICAL is available on GitHub at github.com/xichensf/magical and the Zenodo repository.

### 1.8 Additional Embodiments

One aspect of the present disclosure provides a method for determining whether a subject is afflicted with an antibiotic resistant S. aureses infection or an antibiotic sensitive S. aureses infection. The method comprises obtaining a plurality of discrete attribute values, were each discrete attribute value in the plurality of discrete attribute values represents a transcript abundance of a respective gene in a plurality of genes in a biological sample from the subject, wherein the plurality of genes comprises three or more genes listed in Table 1.14. The plurality of discrete attribute values is inputted into a model comprising a plurality of parameters, where the model applies the plurality of parameters to the plurality of discrete attribute values to generate as output from the model an indication as to whether the subject is afflicted with an antibiotic resistant S. aureses infection or an antibiotic sensitive S. aureses infection.

In some embodiments, the plurality of genes comprises 4, 5, 6, 7, 8, 9, 10, 11, 12, 13, 14, 15, 16, 17, 18, 19, or 20 or more genes listed in Table 1.14. In some embodiments, the plurality of genes comprises 20, 30, 40, 50 or all 53 genes listed in Table 1.14. In some embodiments, the plurality of genes consists of 4, 5, 6, 7, 8, 9, 10, 11, 12, 13, 14, 15, 16, 17, 18, 19, or 20 more genes listed in Table 1.14. In some embodiments, the plurality of genes consists of between 10 and 20, between 10 and 30, between 20 and 40, between 20 and 50, between 5 and 53, between 10 and 53, between 15 and 53, between 20 and 53, between 25 and 53, between 30 and 53, or between 35 and 53 genes listed in Table 1.14.

In some embodiments the plurality of discrete attribute values is obtained by bulk transcriptome sequencing of nucleic acids in the biological sample.

In some embodiments the plurality of discrete attribute values is obtained by single cell transcriptome sequencing of nucleic acids in the biological sample.

In some embodiments, a first gene in the plurality of genes is associated with the cell type CD4_TCM, CD8TE, or CD14_Mono in Table 1.14.

In some embodiments, the method further comprises obtaining, in electronic form, a plurality of sequence reads from the biological sample, where the plurality of sequence reads comprises at least 10,000 RNA sequence reads, and using the plurality of sequence reads to determine each discrete attribute value in the plurality of discrete attribute values. In some such embodiments, each respective sequence read in the plurality of sequence reads is mapped to a reference genome to determine the plurality of abundance values.

In some embodiments, the biological sample is blood, whole blood, or plasma.

In some embodiments, the biological sample comprises a plurality of mRNA molecules and the obtaining the plurality of sequence reads further comprises sequencing the plurality of mRNA molecules using RNA sequencing.

In some embodiments, the plurality of sequence reads comprises at least 100,000, at least 1×10⁶, or at least 1×10⁷ sequence reads.

In some embodiments, the model is selected from the group consisting of: a logistic regression model, a neural network, a support vector machine, a Naive Bayes model, a nearest neighbor model, a boosted trees model, a random forest model, a decision tree, or a clustering model.

In some embodiments, the plurality of parameters comprises 100 or more parameters, 1000 or more parameters, 10,000 or more parameters, 100,000 or more parameters, or 1×10⁶ or more parameters.

In some embodiments, the biological sample comprises serum, urine, cerebrospinal fluid, fecal, saliva, sweat, tears, pleural fluid, pericardial fluid, or peritoneal fluid from the subject.

In some embodiments, the biological sample consists of blood, whole blood, plasma, serum, urine, cerebrospinal fluid, fecal, saliva, sweat, tears, pleural fluid, pericardial fluid, or peritoneal fluid from the subject.

In some embodiments, the method further comprises treating the subject with a drug when the model indicates that the subject has a S. aureses sensitive infection. In some such embodiments the drug is cefazolin, nafcillin, oxacillin, vancomycin, daptomycin, linezolid, or a combination thereof.

Another aspect of the present disclosure provides a method for determining whether a subject is afflicted with COVID-19 in which a plurality of discrete attribute values is obtained. Each discrete attribute value in the plurality of discrete attribute values represents a transcript abundance of a respective gene in a plurality of genes in a biological sample from the subject, where the plurality of genes comprises three or more genes listed in FIG. 60. The plurality of discrete attribute values is inputted into a model comprising a plurality of parameters. The model applies the plurality of parameters to the plurality of discrete attribute values to generate as output from the model an indication as to whether the subject is afflicted with COVID-19.

In some embodiments, the plurality of genes comprises 4, 5, 6, 7, 8, 9, 10, 11, 12, 13, 14, 15, 16, 17, 18, 19, or 20 or more genes listed in FIG. 60. In some embodiments, the plurality of genes comprises 20, 30, 40, 50 or all the genes listed in FIG. 60. In some embodiments, the plurality of genes consists of 4, 5, 6, 7, 8, 9, 10, 11, 12, 13, 14, 15, 16, 17, 18, 19, or 20 more genes listed in FIG. 60. In some embodiments, the plurality of genes consists of between 10 and 20, between 10 and 30, between 20 and 40, between 20 and 50, between 5 and 100, between 10 and 100, between 15 and 200, between 20 and 200, between 25 and 225, between 30 and 225, or between 35 and 225 genes listed in FIG. 60.

In some embodiments the plurality of discrete attribute values is obtained by bulk transcriptome sequencing of nucleic acids in the biological sample.

In some embodiments the plurality of discrete attribute values is obtained by single cell transcriptome sequencing of nucleic acids in the biological sample.

In some embodiments, the method further comprises obtaining, in electronic form, a plurality of sequence reads from the biological sample, where the plurality of sequence reads comprises at least 10,000 RNA sequence reads, and using the plurality of sequence reads to determine each discrete attribute value in the plurality of discrete attribute values. In some such embodiments, each respective sequence read in the plurality of sequence reads is mapped to a reference genome to determine the plurality of abundance values.

In some embodiments, the biological sample is blood, whole blood, or plasma.

In some embodiments, the biological sample comprises a plurality of mRNA molecules and the obtaining the plurality of sequence reads further comprises sequencing the plurality of mRNA molecules using RNA sequencing.

In some embodiments, the plurality of sequence reads comprises at least 100,000, at least 1×10⁶, or at least 1×10⁷ sequence reads.

In some embodiments, the model is selected from the group consisting of: a logistic regression model, a neural network, a support vector machine, a Naive Bayes model, a nearest neighbor model, a boosted trees model, a random forest model, a decision tree, or a clustering model.

In some embodiments, the plurality of parameters comprises 100 or more parameters, 1000 or more parameters, 10,000 or more parameters, 100,000 or more parameters, or 1×10⁶ or more parameters.

In some embodiments, the biological sample comprises serum, urine, cerebrospinal fluid, fecal, saliva, sweat, tears, pleural fluid, pericardial fluid, or peritoneal fluid from the subject.

In some embodiments, the biological sample consists of blood, whole blood, plasma, serum, urine, cerebrospinal fluid, fecal, saliva, sweat, tears, pleural fluid, pericardial fluid, or peritoneal fluid from the subject.

In some embodiments, the method further comprises treating the subject with a drug when the model indicates that the subject has Covid-19. In some embodiments the drug is Nirmatrelvir, Ritonavir, Remdesvir, Molnupiravir, or a combination thereof.

Part 2: Systems and Methods for a Methylation-Based Clock that Enables Accurate Predictions of Time Since Mild SARS-CoV-2 Infection and Provides Insight into Trained Immunity.

### Description.

One aspect of the present disclosure provides a method for predicting a future severity of an infection or inflammatory disease in a subject afflicted with the infection or inflammatory disease in which a plurality of methylation levels is obtained. Each respective methylation level in the plurality of methylation levels represents a corresponding methylation level at one or more CpG sites at a corresponding genetic locus in a plurality of genetic loci in a biological sample obtained from the subject. The plurality of methylation levels is inputted into a model comprising a plurality of parameters. The model applies the plurality of parameters to the plurality of methylation levels to generate as output from the model an indication as to future severity of an infection or inflammatory disease in the subject.

Another aspect of the present disclosure provides a method for predicting susceptibility a subject has to an infection in a subject presently free of the infection in which a plurality of methylation levels is obtained. Each respective methylation level in the plurality of methylation levels represents a corresponding methylation level at one or more CpG sites at a corresponding genetic locus in a plurality of genetic loci in a biological sample obtained from the subject. The plurality of methylation levels is inputted into a model comprising a plurality of parameters. The model applies the plurality of parameters to the plurality of methylation levels to generate as output from the model the susceptibility the subject has to incurring a severe form of the infection upon exposure to the invention.

Another aspect of the present disclosure provides a method for predicting how long a subject has had an infection. The method comprises obtaining a plurality of methylation levels. Each respective methylation level in the plurality of methylation levels represents a corresponding methylation level at one or more CpG sites at a corresponding genetic locus in a plurality of genetic loci in a biological sample obtained from the subject. The plurality of methylation levels is inputted into a model comprising a plurality of parameters. The model applies the plurality of parameters to the plurality of methylation levels to generate as output from the model a period of time the subject has had the infection.

In some embodiments in accordance with Part 2, the infection is a chronic hepatitis C virus infection, chronic human immunodeficiency virus infection, or SARS-CoV-2. In some embodiments in accordance with Part 2, the inflammatory disease is systemic lupus erythematosus, multiple sclerosis, rheumatoid arthritis, or inflammatory bowel disease. In some embodiments in accordance with part 2, each genetic loci in the plurality of genetic loci corresponds to a CpG site in a human genome.

In some embodiments in accordance with part 2, the plurality of genetic loci is five or more loci, 10 or more loci, 20 or more loci, 30 or more loci, 50 or more loci, 100 or more loci, 1000 or more loci, 10,000 or more loci, or 100,000 or more loci. [00307]70. The method of claim 69, wherein at least five genetic loci in the plurality of genetic loci are listed in FIG. 3B.

In some embodiments in accordance with part 2, the biological sample is blood, whole blood, or plasma.

In some embodiments in accordance with part 2, the plurality of methylation levels is obtained from sequencing a plurality of sequence reads of nucleic acids in the biological sample. In some such embodiments this sequencing is bisulfite sequence. In some embodiments the plurality of sequence reads comprises at least 10,000, at least 100,000, at least 1×10⁶, or at least 1×10⁷ sequence reads.

In some embodiments in accordance with part 2, the model is selected from the group consisting of: a logistic regression model, a neural network, a support vector machine, a Naive Bayes model, a nearest neighbor model, a boosted trees model, a random forest model, a decision tree, or a clustering model.

In some embodiments in accordance with part 2, the plurality of parameters comprises 100 or more parameters, 1000 or more parameters, 10,000 or more parameters, 100,000 or more parameters, or 1×10⁶ or more parameters.

In some embodiments in accordance with part 2, the biological sample comprises serum, urine, cerebrospinal fluid, fecal, saliva, sweat, tears, pleural fluid, pericardial fluid, or peritoneal fluid from the subject.

In some embodiments in accordance with part 2, the biological sample consists of blood, whole blood, plasma, serum, urine, cerebrospinal fluid, fecal, saliva, sweat, tears, pleural fluid, pericardial fluid, or peritoneal fluid from the subject.

In some embodiments in accordance with part 2, the infection is SARS-CoV-2 and the plurality of CpG sites comprises 5 or more, 10 or more, 20 or more, 30 or more, 40 or more, or 50 or more CpG sites listed in Tables 2.3 or 2.4.

In some embodiments in accordance with part 2, the infection is SARS-CoV-2 and the plurality of CpG sites consists of 5 or more, 10 or more, 20 or more, 30 or more, 40 or more, or 50 or more CpG sites listed in Tables 2.3 or 2.4.

In some embodiments in accordance with part 2, the infection is SARS-CoV-2 and the plurality of CpG sites consists of between 5 and 100, between 10 and 200, between 15 and 150, between 30 and 500, between 40 and 600, or between 50 and 400 CpG sites listed in Tables 2.3 or 2.4.

In some embodiments in accordance with part 2, the infection is SARS-CoV-2 and 1, 2, 3, 4, 5, 6, 7, 8, 9, 10, 11, 12, 13, 14, 15, 16, 17, 18, 19 or 20 CpG sites in the plurality of CpG sites are indicated to be hypomethylated during First-Control, Mid-Control, EarlyPost-Control, or Late Post-Control in Tables 2.3 or 2.4.

In some embodiments in accordance with part 2, the infection is SARS-CoV-2 and 1, 2, 3, 4, 5, 6, 7, 8, 9, 10, 11, 12, 13, 14, 15, 16, 17, 18, 19 or 20 CpG sites in the plurality of CpG sites are indicated to be hypermethylated during First-Control, Mid-Control, EarlyPost-Control, or Late Post-Control in Tables 2.3 or 2.4.

In some embodiments in accordance with part 2, the infection is SARS-CoV-2 and the plurality of CpG sites comprises 5 or more, 10 or more, 20 or more, 30 or more, 40 or more, or 50 or more CpG sites listed in Tables 2.5 or 2.6.

In some embodiments in accordance with part 2, the infection is SARS-CoV-2 and the plurality of CpG sites consists of 5 or more, 10 or more, 20 or more, 30 or more, 40 or more, or 50 or more CpG sites listed in Tables 2.5 or 2.6.

In some embodiments in accordance with part 2, the infection is SARS-CoV-2 and the plurality of CpG sites consists of between 5 and 100, between 10 and 200, between 15 and 150, between 30 and 500, between 40 and 600, or between 50 and 400 CpG sites listed in Tables 2.5 or 2.6.

In some embodiments in accordance with part 2, the infection is SARS-CoV-2 and 1, 2, 3, 4, 5, 6, 7, 8, 9, 10, 11, 12, 13, 14, 15, 16, 17, 18, 19 or 20 CpG sites in the plurality of CpG sites are indicated to be hypomethylated during Asymptomatic.Control-Symptomatic.Control, First-Symptomatic.First, Asymptomatic.Mid-Symptomatic.Mid, Asymptomatic.EarlyPost-Symptomatic.EarlyPost, or Asymptomatic.LatePost-Symptomatic.LatePost, in Tables 2.5 or 2.6.

In some embodiments in accordance with part 2, the infection is SARS-CoV-2 and 1, 2, 3, 4, 5, 6, 7, 8, 9, 10, 11, 12, 13, 14, 15, 16, 17, 18, 19 or 20 CpG sites in the plurality of CpG sites are indicated to be hypermethylated during Asymptomatic.Control-Symptomatic.Control, First-Symptomatic.First, Asymptomatic.Mid-Symptomatic.Mid, Asymptomatic.EarlyPost-Symptomatic.EarlyPost, or Asymptomatic.LatePost-Symptomatic.LatePost, in Tables 2.5 or 2.6.

In some embodiments in accordance with part 2, each genetic locus in the plurality of genetic loci consists of a single CpG site in the plurality of CpG sites.

In some embodiments in accordance with part 2, each genetic locus in the plurality of genetic loci is less than 1000 nucleotides, less than 500 nucleotides, or less than 300 nucleotides in length.

In some embodiments in accordance with part 2, each genetic locus in the plurality of genetic loci is between 50 and 500 nucleotides in length.

### 2.1. Abstract

DNA methylation comprises a cumulative record of lifetime exposures superimposed on genetically determined markers. Little is known about methylation dynamics in humans following an acute perturbation, such as infection. Here, the temporal trajectory of blood epigenetic remodeling in 133 participants was characterized in a prospective study of young adults before, during, and after asymptomatic and mildly symptomatic SARS-CoV-2 infection. The differential methylation caused by asymptomatic and mildly symptomatic infections were indistinguishable. While differential gene expression largely returned to baseline levels after virus became undetectable, some differentially methylated sites persisted for months of follow up, with a pattern resembling autoimmune or inflammatory disease. These responses were leveraged to construct methylation-based machine learning models that distinguished samples from pre-, during- and post-infection time periods and quantitatively predicted time since infection. The clinical trajectory in the young adults and in a diverse cohort with more sever outcomes was predicted by the similarity of methylation before or early after SARS-CoV-2 infection to the mode-defined postinfection state. Unlike the phenomenon of trained immunity, the postaccute SARS-CoV-2 epigenetic landscape was found to be antiprotective.

### 2.2. Introduction

An individual's pattern of DNA methylation contains a lifetime record of environmental exposures, and has been associated with increased risk for various autoimmune, neurological and metabolic diseases. Methylation-based signatures have been reported to have higher predictive value for future health outcomes than polygenic risk scores (Thompson et al, 2022; Yousefi et al, 2022). DNA methylation has been used to construct lifelong methylation clocks that predict chronological age as well as all-cause mortality (Horvath & Raj, 2018; Lu et al, 2019). While methylation has been linked to diverse phenotypes in association studies, densely sampled longitudinal data that capture intraindividual methylation changes have been limited (Chen et al, 2018; Furukawa et al, 2016).

Here, the present disclosure investigates methylation patterns and dynamics during asymptomatic and mildly symptomatic SARS-CoV-2 infection in healthy young adults. While alterations in blood DNA methylation have been reported after symptomatic SARS-CoV-2 infections (Balnis et al, 2021; Castro de Moura et al, 2021; Corley et al, 2021; Konigsberg et al, 2021; Zhou et al, 2021), the systems and methods of the present disclosure captures the dynamics of methylation changes following asymptomatic infection, giving insights into the long-term memory of environmental exposure and potential disease associations.

### 2.3. Results

Methylome Changes after Infection

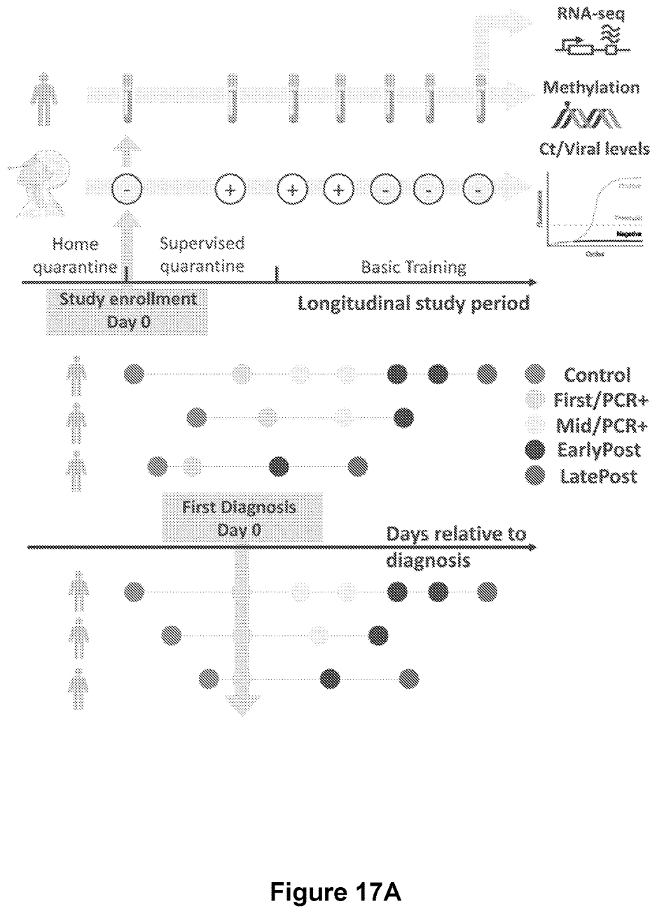

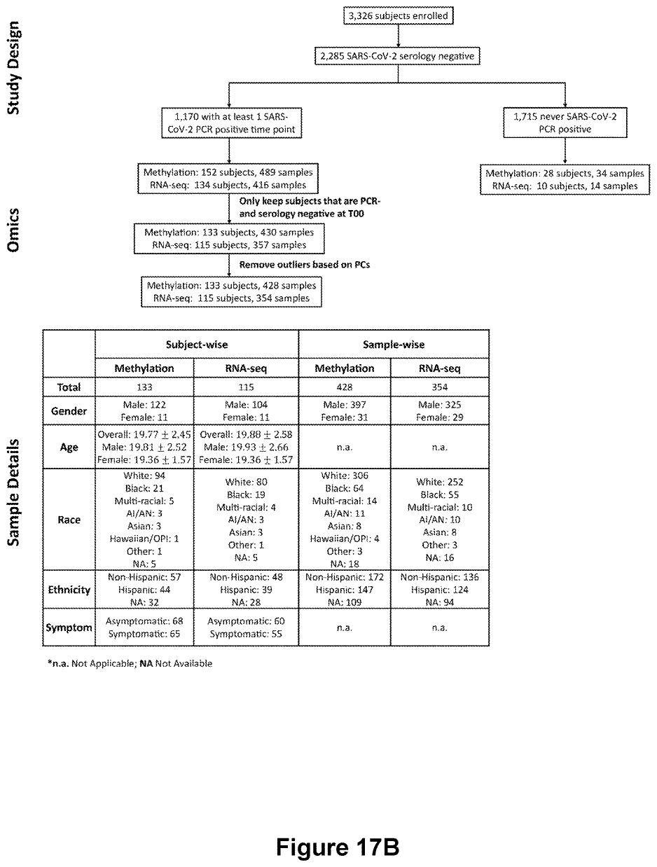

The prospective COVID-19 Health Action Response for Marines (CHARM) study enrolled new US Marine recruits at the beginning of training between May 11-Sep. 7, 2020. Study participants were assessed periodically, including testing for SARS-CoV-2 by nasal swab PCR and blood sampling during an initial two-week supervised quarantine and subsequent basic training (Letizia et al, 2021) (FIG. 17A; see Methods). The cohort was predominantly Caucasian, male and physically fit, with an average age of 19.77±2.45 years (FIG. 17B). Longitudinal blood transcriptome and methylome data obtained from 133 recruits who became infected during the study were analyzed. All infections were either mildly symptomatic (n=65) or asymptomatic (n=68), and none required hospitalization.

The blood samples were grouped relative to day of first diagnosis into the following periods (see FIG. 17A): i) Control (pre-infection), ii) PCR+, which included First (time of first PCR positive test) and Mid (period of subsequent PCR-positive tests), iii) EarlyPost (virus clearance indicated by PCR-negative tests continuing up to 45 days from First), iv) LatePost (PCR-negative tests more than 45 days from First). As seen in Table 2.1, several thousand differentially expressed genes (DEG) were seen at time of first diagnosis compared to pre-infection control levels.

TABLE 2.1
(Top 100 DEG detected over time relative to pre-infection Control. Raw data; FDR < 0.05.
Abbreviations: t, t statistics from limma differential analysis; adj. P. val, adjusted p-value, Raw-No
correction for cell type proportions, FDR < 0.05).

| | Up-Regulated | | | Down-Regulated | | |
|---|---|---|---|---|---|---|
| Rank | Gene | T | Adj. P. Val | Gene | T | Adj. P. Val. |

First-Control

| | | | | | | |
|---|---|---|---|---|---|---|
| 1 | LY6E | 12.7354105 | 6.44E−29 | EIF3L | −10.77173774 | 3.47E−22 |
| 2 | OTOF | 12.1423559 | 9.32E−27 | TIGD3 | −9.998716361 | 1.87E−19 |
| 3 | EPSTI1 | 12.09814737 | 9.32E−27 | FAM168B | −9.881942752 | 4.61E−19 |
| 4 | IFI27 | 12.07151688 | 9.32E−27 | MPZL1 | −9.785601739 | 9.90E−19 |
| 5 | IFI44L | 12.06955929 | 9.32E−27 | RELL1 | −9.473832555 | 1.07E−17 |
| 6 | SIGLEC1 | 11.90276133 | 3.91E−26 | CAMK1D | −9.416149998 | 1.68E−17 |
| 7 | IFI44 | 11.80543728 | 8.56E−26 | RPS6KA5 | −9.189022715 | 8.93E−17 |
| 8 | OAS1 | 11.72551496 | 1.61E−25 | FUZ | −9.148795808 | 1.18E−16 |
| 9 | OAS2 | 11.66110121 | 2.65E−25 | VPS51 | −9.090127863 | 1.76E−16 |
| 10 | HERC6 | 11.57794666 | 5.26E−25 | NUDT3 | −9.011503677 | 3.16E−16 |
| 11 | OAS3 | 11.47313334 | 1.29E−24 | MICAL2 | −8.990861055 | 3.69E−16 |
| 12 | OASL | 11.44367611 | 1.56E−24 | RPGR | −8.865504363 | 9.29E−16 |
| 13 | USP18 | 11.41785855 | 1.84E−24 | BRICD5 | −8.863091858 | 9.30E−16 |
| 14 | DDX60 | 11.40885803 | 1.85E−24 | TP53INP2 | −8.856226354 | 9.74E−16 |
| 15 | SPATS2L | 11.40170283 | 1.85E−24 | CCDC125 | −8.792131544 | 1.55E−15 |
| 16 | CMPK2 | 11.1578076 | 1.70E−23 | SCAP | −8.79173652 | 1.55E−15 |
| 17 | LGALS3BP | 11.12693532 | 2.13E−23 | SPSB3 | −8.7308073 | 2.44E−15 |
| 18 | SHISA5 | 11.10929419 | 2.36E−23 | IGF1R | −8.727592139 | 2.48E−15 |
| 19 | RSAD2 | 11.07981713 | 2.93E−23 | SKI | −8.657891132 | 4.18E−15 |
| 20 | XAF1 | 11.07489805 | 2.93E−23 | NUDT5 | −8.579272689 | 7.45E−15 |
| 21 | RTP4 | 11.01176299 | 4.98E−23 | MAPK8 | −8.520535363 | 1.16E−14 |
| 22 | KLHDC7B | 10.93358017 | 9.75E−23 | CNTNAP3 | −8.475444767 | 1.57E−14 |
| 23 | ISG15 | 10.91656109 | 1.09E−22 | HADHA | −8.448767933 | 1.91E−14 |
| 24 | GALM | 10.83013324 | 2.30E−22 | PIGX | −8.427125698 | 2.24E−14 |
| 25 | ZCCHC2 | 10.82088386 | 2.32E−22 | CCNY | −8.387735303 | 2.94E−14 |
| 26 | IFIT1 | 10.82020266 | 2.32E−22 | EIF3K | −8.352370174 | 3.76E−14 |
| 27 | IRF7 | 10.75971564 | 3.74E−22 | PHF20 | −8.304848427 | 5.31E−14 |
| 28 | TRIM69 | 10.74753846 | 4.03E−22 | BRI3BP | −8.254717802 | 7.53E−14 |
| 29 | IFIT3 | 10.74128518 | 4.12E−22 | TCTN1 | −8.254152286 | 7.53E−14 |
| 30 | MX1 | 10.57430906 | 1.79E−21 | TAF4 | −8.198229757 | 1.11E−13 |
| 31 | IFI6 | 10.52771185 | 2.64E−21 | GRAMD1C | −8.196379512 | 1.12E−13 |
| 32 | IFIH1 | 10.52011341 | 2.74E−21 | FGFR1OP | −8.183408481 | 1.21E−13 |
| 33 | DHX58 | 10.4156808 | 6.73E−21 | IMPA2 | −8.153862516 | 1.48E−13 |
| 34 | ZNF496 | 10.37045134 | 9.75E−21 | RAB40C | −8.153121218 | 1.48E−13 |
| 35 | HERC5 | 10.36301404 | 1.01E−20 | FBL | −8.144926821 | 1.56E−13 |
| 36 | ZBP1 | 10.3057702 | 1.63E−20 | DNAJB5 | −8.1368501 | 1.64E−13 |
| 37 | SLC3A2 | 10.30103443 | 1.66E−20 | ATG4B | −8.132197383 | 1.68E−13 |
| 38 | IFIT5 | 10.26456693 | 2.23E−20 | UNC119B | −8.111938332 | 1.94E−13 |
| 39 | TIMM10 | 10.1909834 | 4.13E−20 | AMPD2 | −8.08822033 | 2.30E−13 |
| 40 | SAMD9 | 10.16063766 | 5.26E−20 | IL1RAP | −8.081001498 | 2.40E−13 |
| 41 | EIF2AK2 | 10.15154283 | 5.51E−20 | RFLNB | −8.054257957 | 2.81E−13 |
| 42 | CNP | 10.14974431 | 5.51E−20 | SERTAD2 | −8.053260255 | 2.82E−13 |
| 43 | ZFYVE26 | 10.08635095 | 9.35E−20 | MAP7 | −8.040379319 | 3.08E−13 |
| 44 | AGRN | 10.0561566 | 1.19E−19 | CLEC9A | −8.03581091 | 3.17E−13 |
| 45 | TRIM14 | 10.00361589 | 1.83E−19 | JADE1 | −8.028369167 | 3.31E−13 |
| 46 | PARP12 | 9.981688123 | 2.12E−19 | KAT8 | −8.002906285 | 3.83E−13 |
| 47 | MT2A | 9.951716574 | 2.69E−19 | ADGRE3 | −7.948918869 | 5.46E−13 |
| 48 | PLSCR1 | 9.902431703 | 4.02E−19 | COQ8A | −7.93272851 | 6.05E−13 |
| 49 | GTPBP2 | 9.882341656 | 4.61E−19 | STK11IP | −7.902830766 | 7.41E−13 |
| 50 | MOV10 | 9.862689774 | 5.33E−19 | ALCAM | −7.898299231 | 7.58E−13 |
| 51 | EPHB2 | 9.824883365 | 7.22E−19 | GNAQ | −7.88414183 | 8.32E−13 |
| 52 | CMTR1 | 9.774174652 | 1.07E−18 | CD1C | −7.866579187 | 9.36E−13 |
| 53 | SAMD9L | 9.749300551 | 1.30E−18 | FAM204A | −7.855176586 | 1.00E−12 |
| 54 | RUFY4 | 9.740059029 | 1.38E−18 | BRD8 | −7.836072864 | 1.15E−12 |
| 55 | TRIM22 | 9.699864378 | 1.91E−18 | CDC123 | −7.809403605 | 1.38E−12 |
| 56 | ABCA1 | 9.697381895 | 1.92E−18 | SEC61A2 | −7.798397383 | 1.47E−12 |
| 57 | IFI35 | 9.680249209 | 2.18E−18 | CFAP45 | −7.794415863 | 1.50E−12 |
| 58 | TRIM5 | 9.642081485 | 2.96E−18 | ENTPD2 | −7.737074768 | 2.20E−12 |
| 59 | SP100 | 9.632903892 | 3.14E−18 | RFX2 | −7.703656896 | 2.74E−12 |
| 60 | PARP10 | 9.608504983 | 3.80E−18 | GMCL1 | −7.674790638 | 3.32E−12 |
| 61 | SERPING1 | 9.56437327 | 5.41E−18 | CNTNAP3B | −7.663162674 | 3.59E−12 |
| 62 | TMEM123 | 9.545782169 | 6.22E−18 | TCP11L2 | −7.644876855 | 4.06E−12 |
| 63 | IFIT2 | 9.543581071 | 6.25E−18 | PTOV1 | −7.637150713 | 4.27E−12 |
| 64 | HELZ2 | 9.509266348 | 8.19E−18 | MAPRE3 | −7.615942741 | 4.85E−12 |
| 65 | CREB3L2 | 9.506349199 | 8.27E−18 | PHOSPHO1 | −7.615591971 | 4.85E−12 |
| 66 | RAB8A | 9.430085329 | 1.51E−17 | FAM214A | −7.611767995 | 4.95E−12 |
| 67 | RUBCN | 9.414445888 | 1.68E−17 | BAIAP3 | −7.589980974 | 5.66E−12 |
| 68 | NT5C3A | 9.403496464 | 1.81E−17 | BTBD7 | −7.57166008 | 6.32E−12 |
| 69 | KIAA1958 | 9.401988009 | 1.81E−17 | HVCN1 | −7.514649679 | 9.13E−12 |
| 70 | IFI16 | 9.371271709 | 2.30E−17 | ENKD1 | −7.497502459 | 1.02E−11 |
| 71 | BST2 | 9.369830782 | 2.30E−17 | SHISA4 | −7.477429247 | 1.16E−11 |
| 72 | ATP13A1 | 9.356368652 | 2.53E−17 | ISL2 | −7.470540275 | 1.21E−11 |
| 73 | SCO2 | 9.328018701 | 3.16E−17 | TESC | −7.466774056 | 1.24E−11 |
| 74 | PML | 9.318604017 | 3.37E−17 | TBL1X | −7.464618429 | 1.25E−11 |
| 75 | REC8 | 9.316670626 | 3.38E−17 | TBC1D14 | −7.454295376 | 1.34E−11 |
| 76 | TRIM38 | 9.268308811 | 4.96E−17 | NECTIN1 | −7.4526944 | 1.35E−11 |
| 77 | FBXO6 | 9.24909242 | 5.74E−17 | ASF1B | −7.450856767 | 1.35E−11 |
| 78 | PSMA6 | 9.239349543 | 6.14E−17 | AGTPBP1 | −7.450676943 | 1.35E−11 |
| 79 | SP140 | 9.233343183 | 6.37E−17 | PI3 | −7.416739265 | 1.69E−11 |
| 80 | CHMP5 | 9.224678761 | 6.76E−17 | UXT | −7.40088079 | 1.86E−11 |
| 81 | SHFL | 9.18519674 | 9.10E−17 | WDR45 | −7.388069171 | 2.02E−11 |
| 82 | IFITM1 | 9.17567176 | 9.73E−17 | MFNG | −7.375815948 | 2.16E−11 |
| 83 | PARP9 | 9.172409023 | 9.88E−17 | SMURF2 | −7.346674546 | 2.60E−11 |
| 84 | IL1RN | 9.137255371 | 1.28E−16 | CEACAM19 | −7.340285875 | 2.70E−11 |
| 85 | DDX60L | 9.123212678 | 1.42E−16 | INPP5K | −7.310591984 | 3.28E−11 |
| 86 | CCDC97 | 9.118035134 | 1.47E−16 | KPNA1 | −7.291265187 | 3.71E−11 |
| 87 | C2 | 9.111553801 | 1.53E−16 | CCDC153 | −7.274352867 | 4.10E−11 |
| 88 | BLZF1 | 9.110265827 | 1.53E−16 | VPS37C | −7.266789747 | 4.28E−11 |
| 89 | MAD2L1BP | 9.093702896 | 1.73E−16 | RAB11B | −7.261464392 | 4.42E−11 |
| 90 | DDX58 | 9.056987382 | 2.28E−16 | TBC1D17 | −7.252678293 | 4.65E−11 |
| 91 | ELF1 | 9.045842893 | 2.47E−16 | FAM107B | −7.252555672 | 4.65E−11 |
| 92 | PLAC8 | 9.033130155 | 2.71E−16 | KBTBD7 | −7.229386603 | 5.38E−11 |
| 93 | PARP14 | 9.019284087 | 3.00E−16 | RAB36 | −7.22505004 | 5.52E−11 |
| 94 | CASP10 | 8.980813719 | 3.96E−16 | EIF3H | −7.224199902 | 5.53E−11 |
| 95 | TDRD7 | 8.977993034 | 4.01E−16 | CERK | −7.21412658 | 5.89E−11 |
| 96 | BRCA2 | 8.960922482 | 4.56E−16 | EIF3F | −7.195289635 | 6.64E−11 |
| 97 | TMX2 | 8.954982886 | 4.73E−16 | RNF103 | −7.167685105 | 7.95E−11 |
| 98 | UBE2L6 | 8.918432131 | 6.27E−16 | RFX3 | −7.159370073 | 8.33E−11 |
| 99 | CYSLTR1 | 8.909518013 | 6.67E−16 | RPN1 | −7.148386342 | 8.92E−11 |
| 100 | TOR1B | 8.871873485 | 8.91E−16 | ERGIC3 | −7.147498315 | 8.94E−11 |

Mid-Control

| | | | | | | |
|---|---|---|---|---|---|---|
| 1 | IFI27 | 14.8038466 | 2.63E−38 | BAG1 | −9.874117884 | 1.11E−18 |
| 2 | EPSTI1 | 13.05438049 | 1.28E−30 | PDZKIIP1 | −9.409147197 | 3.87E−17 |
| 3 | LY6E | 12.47960378 | 2.76E−28 | TP53INP2 | −9.256421626 | 1.21E−16 |
| 4 | MKI67 | 11.48271422 | 3.24E−24 | NUDT3 | −9.22150635 | 1.57E−16 |
| 5 | KLHDC7B | 11.30381716 | 1.39E−23 | ELOB | −9.096504182 | 4.01E−16 |
| 6 | OAS1 | 11.19707581 | 3.14E−23 | AGTPBP1 | −9.079359864 | 4.49E−16 |
| 7 | OTOF | 11.04961195 | 1.06E−22 | EPB42 | −9.072351043 | 4.64E−16 |
| 8 | RRM2 | 10.94678226 | 2.38E−22 | FBXO7 | −9.045452265 | 5.39E−16 |
| 9 | TYMS | 10.85562913 | 4.86E−22 | EMC3 | −8.986701916 | 8.27E−16 |
| 10 | IFI44L | 10.80660336 | 6.83E−22 | BBOF1 | −8.965511176 | 9.46E−16 |
| 11 | OASL | 10.66917644 | 2.16E−21 | ASCC2 | −8.89999031 | 1.55E−15 |
| 12 | ABCA1 | 10.59576647 | 3.82E−21 | FUNDC2 | −8.885352068 | 1.71E−15 |
| 13 | H4C8 | 10.56582487 | 4.61E−21 | CCNY | −8.859298498 | 2.06E−15 |
| 14 | IFI44 | 10.39168337 | 2.02E−20 | SNCA | −8.665919421 | 7.79E−15 |
| 15 | SIGLEC1 | 10.36273544 | 2.44E−20 | FAM168B | −8.629700793 | 1.00E−14 |
| 16 | OAS3 | 10.33027054 | 3.04E−20 | SHISA4 | −8.615402293 | 1.08E−14 |
| 17 | CDC20 | 10.26629337 | 5.03E−20 | FIS1 | −8.599972528 | 1.20E−14 |
| 18 | REC8 | 10.16749136 | 1.13E−19 | TTC25 | −8.585031213 | 1.30E−14 |
| 19 | RSAD2 | 10.14246305 | 1.33E−19 | YBX3 | −8.533297818 | 1.90E−14 |
| 20 | ZCCHC2 | 10.05325953 | 2.74E−19 | TESC | −8.51407544 | 2.18E−14 |
| 21 | BUB1 | 9.980438003 | 4.90E−19 | OR2W3 | −8.476968235 | 2.86E−14 |
| 22 | USP18 | 9.901345573 | 9.22E−19 | TMOD1 | −8.418932263 | 4.29E−14 |
| 23 | OAS2 | 9.855729494 | 1.25E−18 | AMPD2 | −8.247842638 | 1.45E−13 |
| 24 | TK1 | 9.83661 | 1.41E−18 | BLVRB | −8.185959159 | 2.24E−13 |
| 25 | CMPK2 | 9.798033448 | 1.88E−18 | TSPAN5 | −8.153627886 | 2.74E−13 |
| 26 | CYSLTR1 | 9.719851367 | 3.52E−18 | STRADB | −8.153177257 | 2.74E−13 |
| 27 | H2BC5 | 9.671705238 | 5.02E−18 | AKTIS1 | −8.133883871 | 3.12E−13 |
| 28 | TPX2 | 9.669484679 | 5.02E−18 | OPTN | −8.117392533 | 3.49E−13 |
| 29 | JCHAIN | 9.613879388 | 7.74E−18 | MXI1 | −8.111623537 | 3.61E−13 |
| 30 | KIFC1 | 9.493029997 | 2.06E−17 | HADHA | −8.057601533 | 5.10E−13 |
| 31 | CENPF | 9.436246581 | 3.19E−17 | PRDX5 | −8.003321986 | 7.30E−13 |
| 32 | CDT1 | 9.384748418 | 4.60E−17 | CAMK1D | −7.990380157 | 7.87E−13 |
| 33 | DDX60 | 9.329430621 | 7.05E−17 | SELENBP1 | −7.988004664 | 7.93E−13 |
| 34 | CDCA7 | 9.281605306 | 1.01E−16 | SLC4A1 | −7.949438383 | 1.04E−12 |
| 35 | TRIM69 | 9.107769278 | 3.85E−16 | CHMP4B | −7.932847982 | 1.16E−12 |
| 36 | GALM | 9.10011187 | 3.99E−16 | LGALS3 | −7.905883606 | 1.35E−12 |
| 37 | PKMYT1 | 9.056692735 | 5.14E−16 | FBXO9 | −7.905823952 | 1.35E−12 |
| 38 | NUSAP1 | 9.052738746 | 5.19E−16 | FAM210B | −7.860029364 | 1.85E−12 |
| 39 | MCM4 | 8.989977417 | 8.22E−16 | ZBTB44 | −7.835387005 | 2.12E−12 |
| 40 | IFIT1 | 8.964651426 | 9.46E−16 | INPP5K | −7.824897429 | 2.27E−12 |
| 41 | XAF1 | 8.823522517 | 2.68E−15 | GYPC | −7.8152882 | 2.41E−12 |
| 42 | SPATS2L | 8.820581925 | 2.70E−15 | WDR45 | −7.813325497 | 2.42E−12 |
| 43 | ZWINT | 8.816133952 | 2.73E−15 | GALNT1 | −7.804096803 | 2.55E−12 |
| 44 | MX1 | 8.814367932 | 2.73E−15 | NFIX | −7.771067776 | 3.18E−12 |
| 45 | ISG15 | 8.782927431 | 3.44E−15 | PLVAP | −7.731656207 | 4.14E−12 |
| 46 | AGRN | 8.763108058 | 3.95E−15 | STK11IP | −7.729661276 | 4.17E−12 |
| 47 | TXNDC5 | 8.756207423 | 4.10E−15 | PSMF1 | −7.727788382 | 4.19E−12 |
| 48 | HERC6 | 8.714635285 | 5.59E−15 | MICAL2 | −7.6950787 | 5.21E−12 |
| 49 | CCNA2 | 8.690293998 | 6.65E−15 | HAGH | −7.683225357 | 5.63E−12 |
| 50 | SHISA5 | 8.687956777 | 6.66E−15 | TPGS2 | −7.644581321 | 7.23E−12 |
| 51 | IFIT3 | 8.6604254 | 8.00E−15 | OSBP2 | −7.560606619 | 1.23E−11 |
| 52 | TOP2A | 8.622258107 | 1.04E−14 | SPATA6 | −7.537699066 | 1.43E−11 |
| 53 | EPHB2 | 8.588550639 | 1.29E−14 | SERF2 | −7.535685206 | 1.44E−11 |
| 54 | IFI6 | 8.586976392 | 1.29E−14 | GATA1 | −7.532081045 | 1.47E−11 |
| 55 | KIAA1958 | 8.42014487 | 4.29E−14 | KLF13 | −7.526502967 | 1.51E−11 |
| 56 | HERC5 | 8.418678328 | 4.29E−14 | WDR13 | −7.514613515 | 1.62E−11 |
| 57 | ZBTB32 | 8.3742421 | 5.93E−14 | SLC8B1 | −7.512639065 | 1.63E−11 |
| 58 | ZBP1 | 8.355873405 | 6.73E−14 | JAZF1 | −7.467417038 | 2.21E−11 |
| 59 | KLHDC8B | 8.348188768 | 7.05E−14 | BRI3BP | −7.463319011 | 2.24E−11 |
| 60 | RTP4 | 8.309670893 | 9.22E−14 | EIF3L | −7.453496333 | 2.36E−11 |
| 61 | HES4 | 8.309337756 | 9.22E−14 | TBC1D14 | −7.435502473 | 2.62E−11 |
| 62 | IGLL5 | 8.225429242 | 1.69E−13 | FUZ | −7.402554833 | 3.27E−11 |
| 63 | EZH2 | 8.180024891 | 2.32E−13 | VPS51 | −7.379865813 | 3.80E−11 |
| 64 | TRIM5 | 8.174348905 | 2.39E−13 | MPP1 | −7.370311822 | 4.03E−11 |
| 65 | LGALS3BP | 8.107193426 | 3.69E−13 | ALCAM | −7.361519718 | 4.25E−11 |
| 66 | KIF11 | 8.097609001 | 3.91E−13 | TMCO3 | −7.360513616 | 4.26E−11 |
| 67 | BLZF1 | 8.086837064 | 4.20E−13 | RAB3IL1 | −7.355807438 | 4.37E−11 |
| 68 | CD38 | 8.084723067 | 4.22E−13 | OAT | −7.348656927 | 4.54E−11 |
| 69 | MAD2L1BP | 8.034042532 | 6.00E−13 | SLC25A39 | −7.310624146 | 5.85E−11 |
| 70 | EIF2AK2 | 8.011793906 | 7.00E−13 | KAT8 | −7.299307606 | 6.28E−11 |
| 71 | MYLIP | 8.009918465 | 7.02E−13 | HBB | −7.297858248 | 6.31E−11 |
| 72 | CLDND1 | 7.994017998 | 7.74E−13 | UBXN6 | −7.293836716 | 6.45E−11 |
| 73 | TMX2 | 7.924778867 | 1.22E−12 | IGF1R | −7.287652272 | 6.69E−11 |
| 74 | AURKB | 7.919149731 | 1.26E−12 | EIF3K | −7.282470648 | 6.89E−11 |
| 75 | IFIT5 | 7.911135748 | 1.33E−12 | GNAQ | −7.274617813 | 7.14E−11 |
| 76 | CCR5 | 7.877272311 | 1.65E−12 | MBNL3 | −7.242779597 | 8.72E−11 |
| 77 | C2CD3 | 7.847016775 | 2.00E−12 | ST13 | −7.238712421 | 8.91E−11 |
| 78 | IFIH1 | 7.846817125 | 2.00E−12 | BCL2L1 | −7.21825763 | 1.02E−10 |
| 79 | RAB8A | 7.842223606 | 2.03E−12 | PPM1B | −7.217562845 | 1.02E−10 |
| 80 | SERPING1 | 7.842187608 | 2.03E−12 | MAPK8 | −7.181833736 | 1.26E−10 |
| 81 | NCOA3 | 7.806576317 | 2.52E−12 | COPS3 | −7.1783729 | 1.28E−10 |
| 82 | NDC80 | 7.801411355 | 2.58E−12 | TAF4 | −7.17624674 | 1.29E−10 |
| 83 | GMNN | 7.731744512 | 4.14E−12 | RPGR | −7.169677772 | 1.35E−10 |
| 84 | TNFRSF17 | 7.716493798 | 4.51E−12 | PTMS | −7.165489555 | 1.38E−10 |
| 85 | MRPS18B | 7.661889536 | 6.50E−12 | RAB11B | −7.164743714 | 1.38E−10 |
| 86 | UBE2C | 7.650650641 | 6.98E−12 | HVCN1 | −7.155783268 | 1.44E−10 |
| 87 | SP140 | 7.622753719 | 8.38E−12 | MPC2 | −7.132114075 | 1.67E−10 |
| 88 | TIMM10 | 7.606966457 | 9.30E−12 | TCP11L2 | −7.129065219 | 1.70E−10 |
| 89 | MOV10 | 7.602621806 | 9.48E−12 | CCDC124 | −7.111856852 | 1.88E−10 |
| 90 | BRCA2 | 7.602211027 | 9.48E−12 | TAF1 | −7.107332211 | 1.93E−10 |
| 91 | TRIM14 | 7.596768183 | 9.78E−12 | TBC1D25 | −7.105800775 | 1.94E−10 |
| 92 | SSTR3 | 7.593960891 | 9.89E−12 | RELL1 | −7.099889613 | 2.00E−10 |
| 93 | FABP5 | 7.59307292 | 9.89E−12 | TANGO2 | −7.090958145 | 2.11E−10 |
| 94 | TRIM22 | 7.557566876 | 1.25E−11 | CCDC125 | −7.069873431 | 2.37E−10 |
| 95 | NKD1 | 7.524524393 | 1.52E−11 | CCNJL | −7.066306078 | 2.42E−10 |
| 96 | OR52K1 | 7.480942915 | 2.02E−11 | UBL7 | −7.027071247 | 3.09E−10 |
| 97 | ZFYVE26 | 7.465427306 | 2.23E−11 | POLL | −6.997734419 | 3.70E−10 |
| 98 | IFITM1 | 7.460555138 | 2.26E−11 | BTBD7 | −6.994357052 | 3.76E−10 |
| 99 | MZB1 | 7.460332731 | 2.26E−11 | IMPA2 | −6.991228109 | 3.78E−10 |
| 100 | NEXN | 7.448942837 | 2.42E−11 | SLC25A37 | −6.984295476 | 3.94E−10 |

EarlyPost-Control

| | | | | | | |
|---|---|---|---|---|---|---|
| 1 | ZBTB32 | 7.04E+00 | 6.35E−08 | MYBL1 | −6.47E+00 | 1.20E−06 |
| 2 | INSL3 | 5.69E+00 | 3.76E−05 | KLF13 | −6.37E+00 | 1.43E−06 |
| 3 | TNFRSF13B | 5.40E+00 | 9.43E−05 | F2R | −6.07E+00 | 6.76E−06 |
| 4 | EPSTI1 | 5.32E+00 | 1.24E−04 | BRD1 | −5.87E+00 | 1.66E−05 |
| 5 | C7orf61 | 5.29E+00 | 1.34E−04 | IL2RB | −5.58E+00 | 5.97E−05 |
| 6 | ABCA1 | 5.18E+00 | 2.00E−04 | GSAP | −5.48E+00 | 9.02E−05 |
| 7 | CD180 | 5.11E+00 | 2.44E−04 | TAF1 | −5.40E+00 | 9.43E−05 |
| 8 | SREBF1 | 4.92E+00 | 4.26E−04 | BORCS6 | −5.40E+00 | 9.43E−05 |
| 9 | TOP2A | 4.92E+00 | 4.26E−04 | GZMB | −5.40E+00 | 9.43E−05 |
| 10 | PLA2G15 | 4.84E+00 | 4.97E−04 | SH2D1B | −5.38E+00 | 9.72E−05 |
| 11 | FAM222B | 4.84E+00 | 4.97E−04 | PDK4 | −5.25E+00 | 1.56E−04 |
| 12 | STIL | 4.74E+00 | 6.81E−04 | RCSD1 | −5.17E+00 | 2.00E−04 |
| 13 | LETM2 | 4.73E+00 | 6.84E−04 | SMAD7 | −5.16E+00 | 2.00E−04 |
| 14 | GLI1 | 4.70E+00 | 7.80E−04 | SH2D2A | −5.10E+00 | 2.45E−04 |
| 15 | TK1 | 4.67E+00 | 8.46E−04 | KLRF1 | −5.09E+00 | 2.45E−04 |
| 16 | CDC20 | 4.67E+00 | 8.46E−04 | GOLGA8N | −5.07E+00 | 2.61E−04 |
| 17 | IFI27 | 4.63E+00 | 1.02E−03 | SPON2 | −5.06E+00 | 2.61E−04 |
| 18 | POU2AF1 | 4.59E+00 | 1.14E−03 | INIP | −5.03E+00 | 2.96E−04 |
| 19 | IRF4 | 4.57E+00 | 1.22E−03 | SECISBP2L | −5.00E+00 | 3.30E−04 |
| 20 | MKI67 | 4.57E+00 | 1.22E−03 | WRNIP1 | −4.99E+00 | 3.30E−04 |
| 21 | ICOS | 4.54E+00 | 1.28E−03 | MATK | −4.99E+00 | 3.30E−04 |
| 22 | CES4A | 4.54E+00 | 1.28E−03 | RHOU | −4.939732752 | 0.000402855 |
| 23 | CDCA7 | 4.50E+00 | 1.43E−03 | NUDT3 | −4.903854826 | 0.000435379 |
| 24 | NUSAP1 | 4.42E+00 | 1.85E−03 | CD1D | −4.88E+00 | 4.69E−04 |
| 25 | IGLL5 | 4.42E+00 | 1.85E−03 | PDZD4 | −4.86E+00 | 4.97E−04 |
| 26 | JCHAIN | 4.42E+00 | 1.85E−03 | GNPTAB | −4.85E+00 | 4.97E−04 |
| 27 | TPX2 | 4.40E+00 | 1.94E−03 | RNF165 | −4.84E+00 | 4.97E−04 |
| 28 | FGFR1 | 4.38E+00 | 2.00E−03 | CHST2 | −4.82E+00 | 5.38E−04 |
| 29 | ABCG1 | 4.37E+00 | 2.04E−03 | SDE2 | −4.81E+00 | 5.38E−04 |
| 30 | P2RX4 | 4.37E+00 | 2.04E−03 | PTPN11 | −4.81E+00 | 5.38E−04 |
| 31 | MYLIP | 4.36E+00 | 2.08E−03 | MEX3C | −4.80E+00 | 5.44E−04 |
| 32 | TAS1R3 | 4.36E+00 | 2.08E−03 | CDC37 | −4.80E+00 | 5.44E−04 |
| 33 | PSPN | 4.35E+00 | 2.17E−03 | PRF1 | −4.76E+00 | 6.21E−04 |
| 34 | NT5DC2 | 4.27E+00 | 2.80E−03 | PTGDR | −4.68E+00 | 8.31E−04 |
| 35 | TP53BP1 | 4.25E+00 | 2.99E−03 | DENND6A | −4.62E+00 | 1.04E−03 |
| 36 | GOLPH3L | 4.23E+00 | 3.22E−03 | GK5 | −4.60E+00 | 1.14E−03 |
| 37 | PABPC1L | 4.23E+00 | 3.25E−03 | S1PR5 | −4.577073184 | 0.001197337 |
| 38 | LZTS3 | 4.20E+00 | 3.49E−03 | KLRD1 | −4.553895635 | 0.001263242 |
| 39 | TYMS | 4.20E+00 | 3.50E−03 | CCL4 | −4.54E+00 | 1.28E−03 |
| 40 | OR52K1 | 4.19E+00 | 3.52E−03 | NCR1 | −4.53E+00 | 1.33E−03 |
| 41 | RRM2 | 4.19E+00 | 3.53E−03 | LSM14B | −4.52E+00 | 1.34E−03 |
| 42 | KIF11 | 4.15E+00 | 3.91E−03 | STUB1 | −4.51E+00 | 1.43E−03 |
| 43 | TRAF3IP2 | 4.11E+00 | 4.39E−03 | KLF9 | −4.45E+00 | 1.75E−03 |
| 44 | MAML2 | 4.10E+00 | 4.42E−03 | MBNL1 | −4.44E+00 | 1.80E−03 |
| 45 | SLC22A1 | 4.09E+00 | 4.51E−03 | ESRRA | −4.44E+00 | 1.80E−03 |
| 46 | PKMYT1 | 4.08453541 | 0.0046391 | CLIC3 | −4.429427361 | 0.001854666 |
| 47 | EFCAB12 | 4.07245578 | 0.004764 | ENPP4 | −4.42E+00 | 1.85E−03 |
| 48 | TNFRSF17 | 4.03E+00 | 5.62E−03 | NMUR1 | −4.42E+00 | 1.85E−03 |
| 49 | TCEA3 | 4.00E+00 | 5.95E−03 | EDF1 | −4.41E+00 | 1.85E−03 |
| 50 | BNIPL | 4.00E+00 | 5.95E−03 | HIC1 | −4.41E+00 | 1.85E−03 |
| 51 | KYAT1 | 3.97E+00 | 6.55E−03 | LATS1 | −4.389966059 | 0.001971524 |
| 52 | CNKSR1 | 3.97E+00 | 6.55E−03 | AKR1C3 | −4.38E+00 | 2.04E−03 |
| 53 | TXNDC5 | 3.96E+00 | 6.60E−03 | TP53INP2 | −4.36E+00 | 2.08E−03 |
| 54 | ITGA3 | 3.95E+00 | 6.86E−03 | ETV3 | −4.342527175 | 0.002181092 |
| 55 | ST3GAL6 | 3.95E+00 | 6.86E−03 | ZNF518A | −4.338718574 | 0.002192823 |
| 56 | SLC1A4 | 3.95E+00 | 6.86E−03 | SLC30A5 | −4.29E+00 | 2.63E−03 |
| 57 | CENPF | 3.93E+00 | 7.06E−03 | KCTD20 | −4.29E+00 | 2.63E−03 |
| 58 | HCN3 | 3.93E+00 | 7.07E−03 | G0S2 | −4.27E+00 | 2.80E−03 |
| 59 | FBXL16 | 3.922380784 | 0.007280906 | SREK1 | −4.26E+00 | 2.89E−03 |
| 60 | HID1 | 3.92E+00 | 7.35E−03 | METRNL | −4.259952481 | 0.002897088 |
| 61 | RNF175 | 3.90E+00 | 7.84E−03 | SNF8 | −4.22E+00 | 3.33E−03 |
| 62 | SPATS2 | 3.89E+00 | 7.96E−03 | SLC26A2 | −4.21E+00 | 3.34E−03 |
| 63 | ALPK3 | 3.89E+00 | 8.06E−03 | TSPOAP1 | −4.20E+00 | 3.50E−03 |
| 64 | LRRC37B | 3.87E+00 | 8.32E−03 | SLMAP | −4.19E+00 | 3.50E−03 |
| 65 | ATP6V0A1 | 3.85E+00 | 8.67E−03 | CMKLR1 | −4.18E+00 | 3.65E−03 |
| 66 | ARMCX2 | 3.85E+00 | 8.68E−03 | AUTS2 | −4.17E+00 | 3.69E−03 |
| 67 | MYL6B | 3.85E+00 | 8.73E−03 | CCDC85B | −4.17E+00 | 3.70E−03 |
| 68 | PARM1 | 3.84E+00 | 8.84E−03 | SH3BP5 | −4.16E+00 | 3.76E−03 |
| 69 | SLF1 | 3.84E+00 | 8.93E−03 | PRDX5 | −4.16E+00 | 3.76E−03 |
| 70 | C1orf56 | 3.822170389 | 0.0092207 | NCAM1 | −4.16E+00 | 3.80E−03 |
| 71 | SCML4 | 3.769471281 | 0.0108699 | GNLY | −4.15E+00 | 3.94E−03 |
| 72 | C2CD3 | 3.76E+00 | 1.11E−02 | MYCL | −4.14E+00 | 3.95E−03 |
| 73 | INTS8 | 3.758804999 | 0.011127991 | ZBTB21 | −4.14E+00 | 3.96E−03 |
| 74 | TBCK | 3.75E+00 | 1.14E−02 | DUSP2 | −4.12E+00 | 4.24E−03 |
| 75 | AP3M2 | 3.74E+00 | 1.18E−02 | NEIL1 | −4.11E+00 | 4.36E−03 |
| 76 | CDC42EP3 | 3.72E+00 | 1.25E−02 | ZNF830 | −4.11E+00 | 4.40E−03 |
| 77 | EZH2 | 3.72E+00 | 1.25E−02 | HNRNPA0 | −4.10E+00 | 4.42E−03 |
| 78 | CENPE | 3.712769803 | 0.0125262 | NEDD8 | −4.10E+00 | 4.46E−03 |
| 79 | ANKRD55 | 3.70E+00 | 1.32E−02 | ARFRP1 | −4.08E+00 | 4.75E−03 |
| 80 | PTPRO | 3.68E+00 | 1.37E−02 | TBCB | −4.08E+00 | 4.75E−03 |
| 81 | OSBPL10 | 3.68E+00 | 1.38E−02 | PDE4D | −4.06E+00 | 5.03E−03 |
| 82 | PYGM | 3.67E+00 | 1.39E−02 | LPCAT1 | −4.05E+00 | 5.17E−03 |
| 83 | SYNGAP1 | 3.67E+00 | 1.39E−02 | CCNT2 | −4.04E+00 | 5.33E−03 |
| 84 | ZNF202 | 3.67E+00 | 1.40E−02 | ZBTB16 | −4.02E+00 | 5.79E−03 |
| 85 | ZC3H12D | 3.65E+00 | 1.42E−02 | PPM1B | −4.01E+00 | 5.86E−03 |
| 86 | SLAMF1 | 3.65E+00 | 1.42E−02 | PER3 | −4.01E+00 | 5.88E−03 |
| 87 | ANKRD36C | 3.65E+00 | 1.42E−02 | ZBTB14 | −4.00E+00 | 5.95E−03 |
| 88 | KIFC1 | 3.65E+00 | 1.43E−02 | HIGD1A | −4.00E+00 | 5.95E−03 |
| 89 | MFGE8 | 3.64E+00 | 1.44E−02 | KCTD10 | −3.99E+00 | 6.05E−03 |
| 90 | ACVR2A | 3.64E+00 | 1.48E−02 | CREBZF | −3.99E+00 | 6.13E−03 |
| 91 | GPT2 | 3.63E+00 | 1.49E−02 | KCTD18 | −3.97E+00 | 6.52E−03 |
| 92 | MED31 | 3.63E+00 | 1.49E−02 | EAF1 | −3.953351461 | 0.006793564 |
| 93 | S1PR3 | 3.63E+00 | 1.49E−02 | CNOT6L | −3.95E+00 | 6.79E−03 |
| 94 | HDAC5 | 3.63E+00 | 1.49E−02 | RNF6 | −3.94E+00 | 6.87E−03 |
| 95 | SSTR3 | 3.629124027 | 0.0148778 | PFKFB2 | −3.940986999 | 0.006886166 |
| 96 | CCR9 | 3.62E+00 | 1.51E−02 | SNX18 | −3.898749591 | 0.007856223 |
| 97 | SEL1L3 | 3.61E+00 | 1.55E−02 | TGFBR3 | −3.90E+00 | 7.90E−03 |
| 98 | CDT1 | 3.61E+00 | 1.56E−02 | SLC16A6 | −3.89E+00 | 8.06E−03 |
| 99 | SLC25A23 | 3.61E+00 | 1.56E−02 | YTHDC2 | −3.88E+00 | 8.24E−03 |
| 100 | RNF185 | 3.61 | 0.0156 | TSSC4 | −3.88 | 0.00829 |

Late-Post Control

| | | | | | | |
|---|---|---|---|---|---|---|
| 1 | ZFC3H1 | 3.98186942 | 0.0180675 | KIR2DL1 | −4.844532692 | 0.001552291 |
| 2 | TNFRSF17 | 3.980218302 | 0.0180675 | WRNIP1 | −4.844181258 | 0.001552291 |
| 3 | JAK3 | 3.966586325 | 0.0183699 | FGFBP2 | −4.681461263 | 0.003115043 |
| 4 | NCOA3 | 3.903499195 | 0.0231039 | KLRD1 | −4.631038645 | 0.003662594 |
| 5 | TNRC6B | 3.851112499 | 0.0258741 | SPIN1 | −4.558162921 | 0.004450366 |
| 6 | JCHAIN | 3.845455443 | 0.0258741 | PDZD4 | −4.548625268 | 0.004450366 |
| 7 | NFKBIZ | 3.820271975 | 0.0273110 | SPON2 | −4.546907922 | 0.004450366 |
| 8 | TOP2A | 3.782837699 | 0.0287496 | HS6ST1 | −4.481517766 | 0.004920695 |
| 9 | NUSAP1 | 3.770339681 | 0.0287878 | SOX13 | −4.46975871 | 0.004920695 |
| 10 | SCARF1 | 3.763728209 | 0.0287878 | PRSS23 | −4.465323901 | 0.004920695 |
| 11 | IRF4 | 3.760889617 | 0.0287878 | BRD1 | −4.453701262 | 0.004920695 |
| 12 | PABPC1L | 3.742196668 | 0.0293744 | KLRF1 | −4.448932311 | 0.004920695 |
| 13 | SLC38A2 | 3.705372009 | 0.031451 | POLR3H | −4.42144212 | 0.005344588 |
| 14 | ZWINT | 3.699440951 | 0.0316885 | NMUR1 | −4.392476094 | 0.005848833 |
| 15 | GABBR1 | 3.697138853 | 0.0316885 | CLIC3 | −4.380108986 | 0.005950761 |
| 16 | NFKB2 | 3.689188357 | 0.0316885 | TSPOAP1 | −4.312092062 | 0.007747511 |
| 17 | MSS51 | 3.688421384 | 0.0316885 | RNF165 | −4.300846608 | 0.007857989 |
| 18 | AQP3 | 3.635795444 | 0.0355428 | FASLG | −4.280477535 | 0.008034687 |
| 19 | CDC20 | 3.628388384 | 0.0360303 | ENOPH1 | −4.262653924 | 0.008410994 |
| 20 | ITGA7 | 3.622869581 | 0.0360303 | SH2D2A | −4.203919767 | 0.010277307 |
| 21 | ELMO2 | 3.619063035 | 0.0360303 | MEX3C | −4.202166264 | 0.010277307 |
| 22 | SETD5 | 3.581282639 | 0.0391005 | MYBL1 | −4.189518742 | 0.010539466 |
| 23 | ETFBKMT | 3.577095788 | 0.0391005 | CACNA2D2 | −4.143588805 | 0.012430821 |
| 24 | TXNDC5 | 3.566113746 | 0.0400111 | HOPX | −4.133997927 | 0.012430821 |
| 25 | CCDC88A | 3.556071801 | 0.0411697 | PDGFD | −4.131370425 | 0.012430821 |
| 26 | OR52K1 | 3.537386498 | 0.0432933 | AUTS2 | −4.105464642 | 0.013502175 |
| 27 | KLHL6 | 3.534271532 | 0.0432933 | SH2D1B | −4.099576881 | 0.013502175 |
| 28 | ZC3H6 | 3.525677231 | 0.0441314 | NCAM1 | −4.082589307 | 0.01399559 |
| 29 | MARS1 | 3.497382005 | 0.0472971 | ZNF57 | −4.079371003 | 0.01399559 |
| 30 | NEMF | 3.493732396 | 0.0472971 | ACADVL | −4.035463827 | 0.01603874 |
| 31 | PHF23 | 3.469256787 | 0.0485318 | ITGAV | −4.001195132 | 0.017673145 |
| 32 | CLEC1A | 3.447116161 | 0.0499527 | PRPF31 | −3.975491651 | 0.018067554 |
| 33 | ZFC3H1 | 3.98186942 | 0.0180675 | RNF6 | −3.900398383 | 0.023103962 |
| 34 | TNFRSF17 | 3.980218302 | 0.0180675 | PLEKHF1 | −3.895906905 | 0.023103962 |
| 35 | JAK3 | 3.966586325 | 0.0183699 | AKR1C3 | −3.886331017 | 0.0235736 |
| 36 | NCOA3 | 3.903499195 | 0.0231040 | DLG5 | −3.859464859 | 0.025780511 |
| 37 | TNRC6B | 3.851112499 | 0.0258741 | TKTL1 | −3.849458302 | 0.025874127 |
| 38 | JCHAIN | 3.845455443 | 0.0258741 | PRF1 | −3.835610922 | 0.026457823 |
| 39 | NFKBIZ | 3.820271975 | 0.0273110 | FAM50A | −3.819323594 | 0.027311025 |
| 40 | TOP2A | 3.782837699 | 0.0287496 | PHLDB2 | −3.803830414 | 0.02802852 |
| 41 | NUSAP1 | 3.770339681 | 0.0287878 | ZBTB16 | −3.797727239 | 0.02802852 |
| 42 | SCARF1 | 3.763728209 | 0.0287878 | KCTD10 | −3.796852998 | 0.02802852 |
| 43 | | | | SDE2 | −3.795079817 | 0.02802852 |
| 44 | | | | KLF9 | −3.793078524 | 0.02802852 |
| 45 | | | | NCK1 | −3.778121167 | 0.028787849 |
| 46 | | | | GPATCH3 | −3.769167281 | 0.028787849 |
| 47 | | | | B3GALT6 | −3.767532749 | 0.028787849 |
| 48 | | | | CLDND2 | −3.752441287 | 0.029188103 |
| 49 | | | | PER3 | −3.750508271 | 0.029188103 |
| 50 | | | | TCEAL3 | −3.744994421 | 0.029374408 |
| 51 | | | | C1orf21 | −3.734985926 | 0.029826302 |
| 52 | | | | CEP250 | −3.728890745 | 0.030157973 |
| 53 | | | | ZMYND11 | −3.723512656 | 0.03034952 |
| 54 | | | | STK26 | −3.71827661 | 0.03034952 |
| 55 | | | | MTMR12 | −3.717743487 | 0.03034952 |
| 56 | | | | MEX3D | −3.693470803 | 0.031688529 |
| 57 | | | | CDC37 | −3.685466779 | 0.031693855 |
| 58 | | | | ERBB2 | −3.66719379 | 0.032990648 |
| 59 | | | | ADGRG1 | −3.666200487 | 0.032990648 |
| 60 | | | | TGFBR3 | −3.664643132 | 0.032990648 |
| 61 | | | | S1PR5 | −3.663629213 | 0.032990648 |
| 62 | | | | TAF1 | −3.656153733 | 0.033346455 |
| 63 | | | | FCRL6 | −3.655297404 | 0.033346455 |
| 64 | | | | TTC16 | −3.623948311 | 0.03603034 |
| 65 | | | | MYLK | −3.618922087 | 0.03603034 |
| 66 | | | | ENPP4 | −3.615890202 | 0.036091517 |
| 67 | | | | EXO5 | −3.613295615 | 0.036096298 |
| 68 | | | | ZNF720 | −3.606707843 | 0.036651803 |
| 69 | | | | R3HCC1 | −3.584755959 | 0.039100481 |
| 70 | | | | MC1R | −3.580843196 | 0.039100481 |
| 71 | | | | ARHGAP35 | −3.578173667 | 0.039100481 |
| 72 | | | | FAM53B | −3.570368844 | 0.039735174 |
| 73 | | | | AGAP1 | −3.551234728 | 0.041549801 |
| 74 | | | | TMEM141 | −3.533159379 | 0.043293335 |
| 75 | | | | CMKLR1 | −3.510408931 | 0.046061529 |
| 76 | | | | RABL6 | −3.509519824 | 0.046061529 |
| 77 | | | | GSAP | −3.493870399 | 0.047297102 |
| 78 | | | | TP53INP2 | −3.492681976 | 0.047297102 |
| 79 | | | | HIGD1A | −3.489681574 | 0.047297102 |
| 80 | | | | PPM1L | −3.486981346 | 0.047297102 |
| 81 | | | | TTC38 | −3.486873889 | 0.047297102 |
| 82 | | | | ZCCHC14 | −3.483511599 | 0.047447502 |
| 83 | | | | PLVAP | −3.481737543 | 0.047447502 |
| 84 | | | | ZNF441 | −3.475257786 | 0.047865483 |
| 85 | | | | ZBTB9 | −3.475128686 | 0.047865483 |
| 86 | | | | TLE1 | −3.458007605 | 0.049892749 |
| 87 | | | | P3H4 | −3.457533525 | 0.049892749 |
| 88 | | | | PTGDR | −3.454435652 | 0.049952763 |
| 89 | | | | AJM1 | −3.450407513 | 0.049952763 |
| 90 | | | | KIR2DL1 | −4.844532692 | 0.001552291 |
| 91 | | | | WRNIP1 | −4.844181258 | 0.001552291 |
| 92 | | | | FGFBP2 | −4.681461263 | 0.003115043 |
| 93 | | | | KLRD1 | −4.631038645 | 0.003662594 |
| 94 | | | | SPIN1 | −4.558162921 | 0.004450366 |
| 95 | | | | PDZD4 | −4.548625268 | 0.004450366 |
| 96 | | | | SPON2 | −4.546907922 | 0.004450366 |
| 97 | | | | HS6ST1 | −4.481517766 | 0.004920695 |
| 98 | | | | SOX13 | −4.46975871 | 0.004920695 |
| 99 | | | | PRSS23 | −4.465323901 | 0.004920695 |
| 100 | | | | BRD1 | −4.453701262 | 0.004920695 |

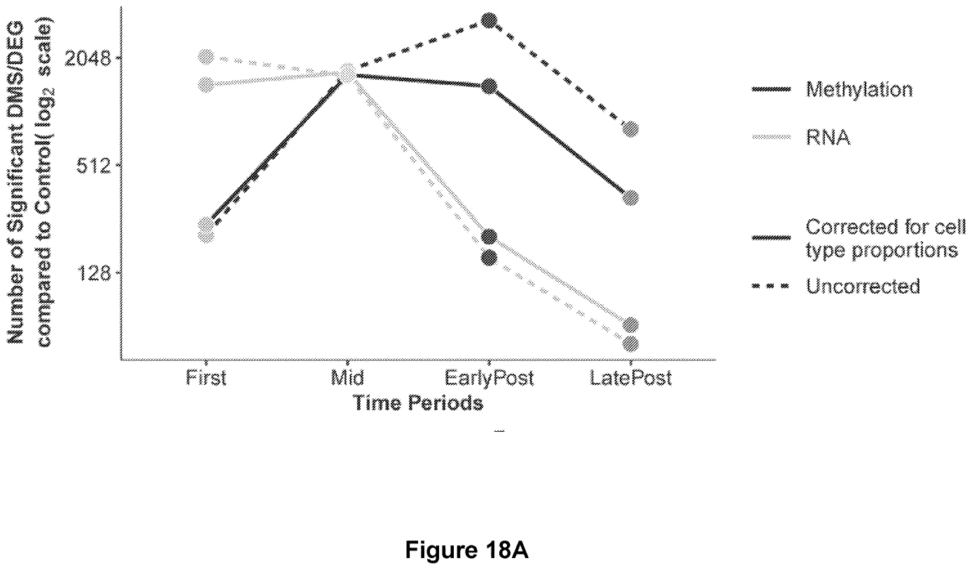

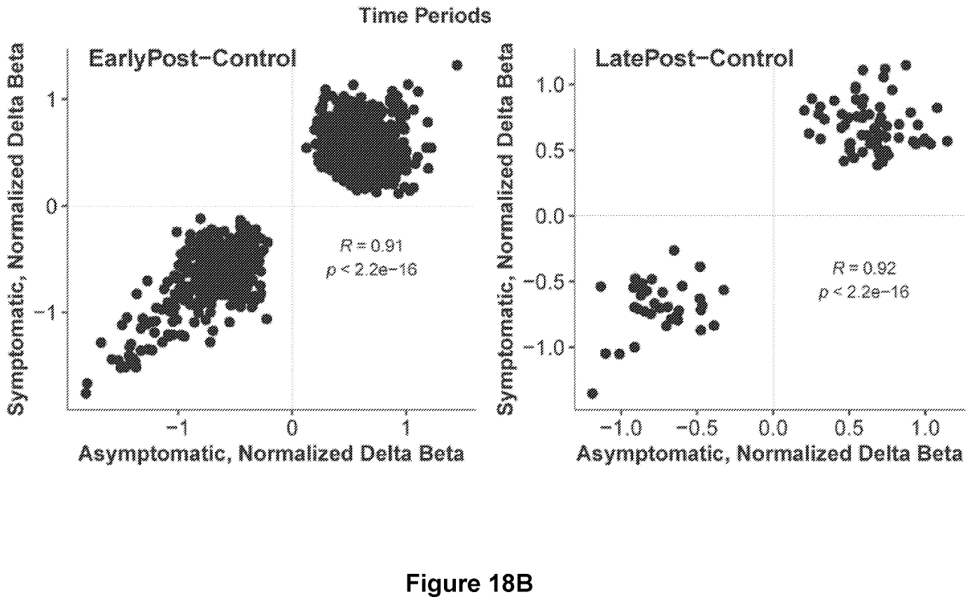

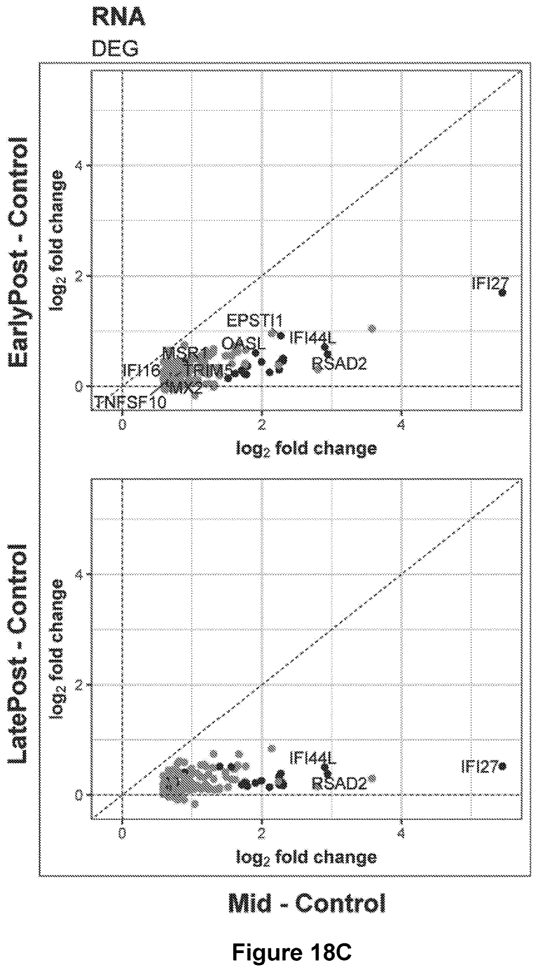

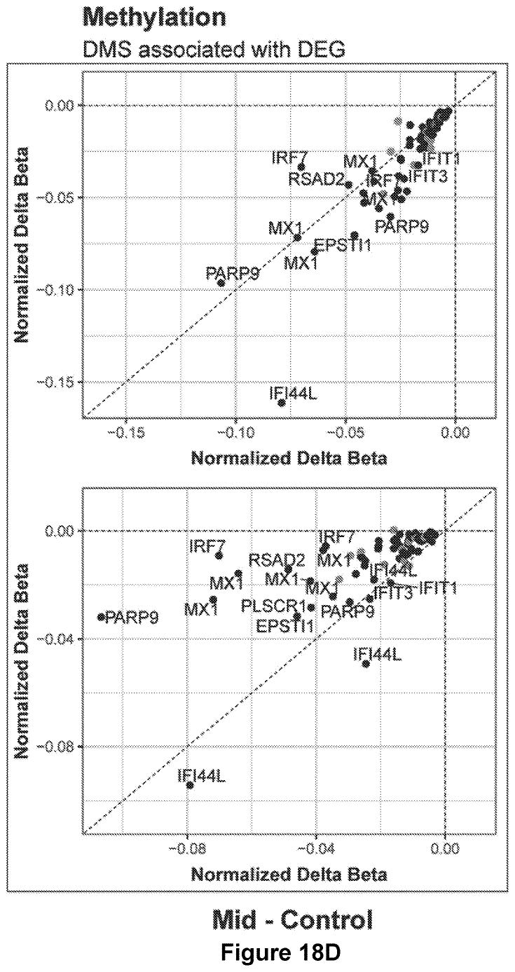

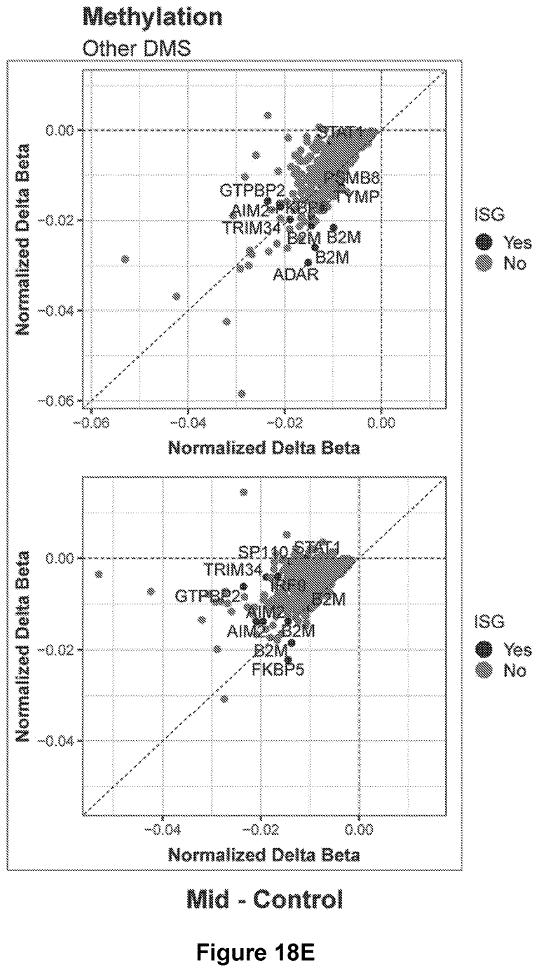

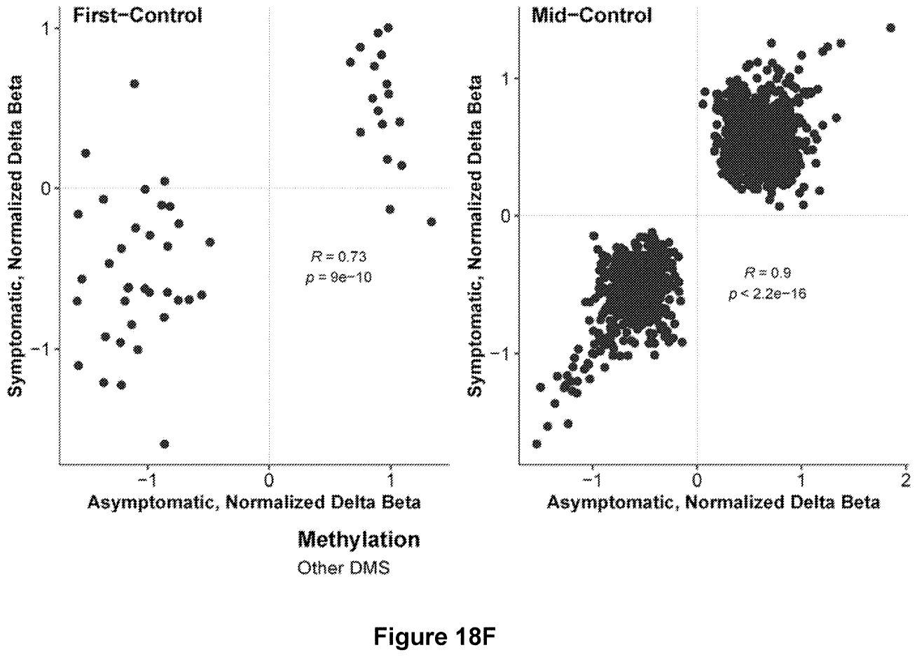

The number of DEG detected at EarlyPost vs. Control was greatly reduced, and few were detected by LatePost. The total number of differentially methylated sites (DMS) in blood DNA peaked later than the DEG, and a large number of DMS were still observed in the periods after PCR positivity (FIG. 18A). Changes in blood cell type proportions occur during SARS-CoV-2 infection (Liu et al, 2020), which may affect the detection of DEG and DMS. Computational cell type deconvolution of both the RNA-seq and methylation data showed concordant changes in the predicted proportions of B cells, T cell subtypes, and NK cells following infection (See Figure Si of Mao et al., 2023, “A methylation clock model of mild SARS-CoV-2 infection provides insight into immune dysregulation,” Molecular Systems Biology 19 e:11361 which is hereby incorporated by reference). The number of DEG and DMS detected over time were similar when analyzing raw data, when correcting for changes in cell type proportions, and when summarizing up- and down-regulation events separately (FIG. 18B). See also Fig. EV2A of Mao et al., 2023, “A methylation clock model of mild SARS-CoV-2 infection provides insight into immune dysregulation,” Molecular Systems Biology 19 e:11361 which is hereby incorporated by reference, and Tables 2.1, 2.2, 2.3 and 2.4).

TABLE 2.2
(Top 100 DEG detected over time relative to pre-infection Control. Data were corrected for cell type
proportions; false discovery rate < 0.05)

| | Up-Regulated | | | Down-Regulated | | |
|---|---|---|---|---|---|---|
| Rank | Gene | T | Adj. P. Val | Gene | T | Adj. P. Val. |

First-Control

| | | | | | | |
|---|---|---|---|---|---|---|
| 1 | IFI27 | 12.99144366 | 6.71E−28 | EIF3L | −10.63405456 | 3.14E−20 |
| 2 | LY6E | 12.73849116 | 3.18E−27 | VPS51 | −10.4684031 | 9.25E−20 |
| 3 | EPSTI1 | 11.92001578 | 2.72E−24 | FBL | −10.09449223 | 1.44E−18 |
| 4 | SHISA5 | 11.53207452 | 5.67E−23 | HADHA | −9.355421137 | 2.72E−16 |
| 5 | OTOF | 11.45175233 | 8.97E−23 | EIF3K | −9.204800733 | 7.34E−16 |
| 6 | SIGLEC1 | 11.26811506 | 3.52E−22 | MPZL1 | −9.071896509 | 1.83E−15 |
| 7 | OASL | 11.17483429 | 6.61E−22 | TIGD3 | −8.877516805 | 6.61E−15 |
| 8 | IFI44L | 11.15590161 | 6.78E−22 | MICAL2 | −8.763029012 | 1.41E−14 |
| 9 | OAS1 | 11.09152192 | 1.03E−21 | FAM168B | −8.650095976 | 2.87E−14 |
| 10 | IFI44 | 10.84929879 | 6.95E−21 | IGF1R | −8.387690414 | 1.67E−13 |
| 11 | ABCA1 | 10.74998626 | 1.43E−20 | AMPD2 | −8.274488905 | 3.50E−13 |
| 12 | OAS2 | 10.64846118 | 3.02E−20 | NUDT3 | −8.257533749 | 3.81E−13 |
| 13 | OAS3 | 10.56221891 | 5.24E−20 | GNAQ | −8.196588004 | 5.34E−13 |
| 14 | SPATS2L | 10.50701555 | 7.66E−20 | EIF3H | −8.132999975 | 7.82E−13 |
| 15 | ZCCHC2 | 10.49755303 | 7.75E−20 | CAMK1D | −8.055687956 | 1.24E−12 |
| 16 | HERC6 | 10.4044129 | 1.47E−19 | RELL1 | −7.974616483 | 2.02E−12 |
| 17 | USP18 | 10.33001419 | 2.53E−19 | STK11IP | −7.959073822 | 2.22E−12 |
| 18 | DDX60 | 10.31900266 | 2.63E−19 | RPS6KA5 | −7.824625223 | 5.17E−12 |
| 19 | GALM | 10.20154836 | 6.43E−19 | PGK1 | −7.776644254 | 7.05E−12 |
| 20 | IL1RN | 10.05915994 | 1.83E−18 | AGTPBP1 | −7.687237155 | 1.19E−11 |
| 21 | CMPK2 | 10.03115445 | 2.19E−18 | IL1RAP | −7.682428552 | 1.22E−11 |
| 22 | KLHDC7B | 9.903437866 | 5.76E−18 | ADGRE3 | −7.449381717 | 5.23E−11 |
| 23 | RSAD2 | 9.886252085 | 6.34E−18 | RPGR | −7.410136617 | 6.68E−11 |
| 24 | ISG15 | 9.813315073 | 1.08E−17 | CCDC125 | −7.398736193 | 7.15E−11 |
| 25 | TRIM69 | 9.79917468 | 1.16E−17 | CRTAP | −7.36453366 | 8.76E−11 |
| 26 | IFIT3 | 9.79529248 | 1.16E−17 | TP53INP2 | −7.327181613 | 1.10E−10 |
| 27 | IFIT1 | 9.718718178 | 2.04E−17 | TBC1D14 | −7.32472934 | 1.11E−10 |
| 28 | XAF1 | 9.684171791 | 2.58E−17 | SHISA4 | −7.308251295 | 1.21E−10 |
| 29 | RTP4 | 9.649642885 | 3.27E−17 | TESC | −7.283854145 | 1.40E−10 |
| 30 | BCL2A1 | 9.593290008 | 4.91E−17 | BRI3BP | −7.269107126 | 1.49E−10 |
| 31 | CARD6 | 9.570594399 | 5.68E−17 | EIF3F | −7.268557377 | 1.49E−10 |
| 32 | IRF7 | 9.560608834 | 5.96E−17 | BRICD5 | −7.208611216 | 2.13E−10 |
| 33 | IFIH1 | 9.362447901 | 2.65E−16 | FAM204A | −7.152115073 | 2.96E−10 |
| 34 | MX1 | 9.296771831 | 4.13E−16 | TCP11L2 | −7.082000718 | 4.50E−10 |
| 35 | DDIT3 | 9.293706234 | 4.13E−16 | BAG1 | −7.081186589 | 4.50E−10 |
| 36 | MYLIP | 9.286342972 | 4.26E−16 | KBTBD7 | −7.0794418 | 4.51E−10 |
| 37 | LGALS3BP | 9.264901095 | 4.89E−16 | NUDT5 | −7.041686153 | 5.66E−10 |
| 38 | ZBP1 | 9.221455932 | 6.63E−16 | UBXN11 | −7.03956463 | 5.71E−10 |
| 39 | IFIT5 | 9.12459234 | 1.31E−15 | CCNY | −7.036591071 | 5.79E−10 |
| 40 | ZFYVE26 | 9.119126086 | 1.34E−15 | CNTNAP3 | −7.019492821 | 6.41E−10 |
| 41 | IFI6 | 9.071211469 | 1.83E−15 | UNC119B | −6.990018854 | 7.54E−10 |
| 42 | DDAH2 | 8.986670849 | 3.37E−15 | FUZ | −6.969738189 | 8.47E−10 |
| 43 | PLSCR1 | 8.983224078 | 3.39E−15 | UXT | −6.933260609 | 0.000000001 |
| 44 | RAB8A | 8.966450493 | 3.77E−15 | C12orf10 | −6.858061122 | 1.60E−09 |
| 45 | HERC5 | 8.957174064 | 3.93E−15 | AHR | −6.856178653 | 1.61E−09 |
| 46 | SP100 | 8.955341837 | 3.93E−15 | SLC31A1 | −6.831473153 | 1.82E−09 |
| 47 | EIF2AK2 | 8.935502765 | 4.47E−15 | PARK7 | −6.798556973 | 2.22E−09 |
| 48 | SLC3A2 | 8.886561568 | 6.30E−15 | SKI | −6.778000299 | 2.50E−09 |
| 49 | LRFN1 | 8.817568921 | 1.01E−14 | IMPA2 | −6.761233708 | 2.76E−09 |
| 50 | ZNF496 | 8.789064977 | 1.22E−14 | ERGIC3 | −6.71472953 | 3.62E−09 |
| 51 | AGRN | 8.776090595 | 1.32E−14 | RTN1 | −6.705272912 | 3.78E−09 |
| 52 | AKIRIN2 | 8.768875351 | 1.37E−14 | ALCAM | −6.686712564 | 4.18E-09 |
| 53 | CD38 | 8.723810213 | 1.84E−14 | PTAFR | −6.67841406 | 4.34E−09 |
| 54 | C2CD3 | 8.722136325 | 1.84E−14 | SPSB3 | −6.656869354 | 4.91E−09 |
| 55 | SAMD9 | 8.719410687 | 1.84E−14 | MATK | −6.644890312 | 5.23E−09 |
| 56 | DDX60L | 8.667387493 | 2.65E−14 | EIF4B | −6.638874345 | 5.41E−09 |
| 57 | DHX58 | 8.662055828 | 2.71E−14 | HOPX | −6.635601774 | 5.49E−09 |
| 58 | GTPBP2 | 8.651933211 | 2.87E−14 | EIF3E | −6.594948031 | 6.88E−09 |
| 59 | CHMP5 | 8.588612458 | 4.42E−14 | KAT8 | −6.582208697 | 7.34E−09 |
| 60 | MKI67 | 8.491475542 | 8.77E−14 | VPS37C | −6.579084364 | 7.45E−09 |
| 61 | H2BC4 | 8.453399613 | 1.14E−13 | SCAP | −6.554594075 | 8.49E−09 |
| 62 | HELZ2 | 8.439288262 | 1.24E−13 | CPPED1 | −6.514926329 | 1.06E−08 |
| 63 | KIAA1958 | 8.437061905 | 1.24E−13 | PHOSPHO1 | −6.487252125 | 1.23E−08 |
| 64 | NTNG2 | 8.434279884 | 1.25E−13 | MAPK8 | −6.473220802 | 1.32E−08 |
| 65 | TRIM5 | 8.42964938 | 1.27E−13 | VENTX | −6.472402876 | 1.32E−08 |
| 66 | MRPS18B | 8.410446126 | 1.44E−13 | SLC46A2 | −6.471733763 | 1.33E−08 |
| 67 | IFIT2 | 8.361457049 | 1.98E−13 | MFNG | −6.464014448 | 1.38E−08 |
| 68 | IFI16 | 8.360172432 | 1.98E−13 | RFLNB | −6.453501687 | 1.47E−08 |
| 69 | ARHGEF11 | 8.348258227 | 2.13E−13 | FCGRT | −6.448058802 | 1.49E−08 |
| 70 | EPHB2 | 8.327948338 | 2.43E−13 | ELOB | −6.440152246 | 1.56E−08 |
| 71 | NMI | 8.269010433 | 3.60E−13 | MAPK1 | −6.437432941 | 1.57E−08 |
| 72 | ITPRIP | 8.266720802 | 3.61E−13 | RPL4 | −6.404456811 | 1.87E−08 |
| 73 | REC8 | 8.252149253 | 3.91E−13 | PPM1F | −6.373070891 | 2.22E−08 |
| 74 | RRM2 | 8.238890639 | 4.24E−13 | CYBRD1 | −6.36207991 | 2.35E−08 |
| 75 | CREB3L2 | 8.23148973 | 4.42E−13 | ASCC2 | −6.337435977 | 2.68E−08 |
| 76 | TMX2 | 8.225791505 | 4.54E−13 | CDC123 | −6.325434241 | 2.85E−08 |
| 77 | TIMM10 | 8.223356289 | 4.57E−13 | WDR45 | −6.324330195 | 2.86E−08 |
| 78 | SP140 | 8.216405556 | 4.75E−13 | TTC25 | −6.303332856 | 3.22E−08 |
| 79 | ELF1 | 8.200108383 | 5.26E−13 | RPN1 | −6.299539179 | 3.28E−08 |
| 80 | GLRX | 8.189732335 | 5.54E−13 | KCNC3 | −6.292304098 | 3.41E−08 |
| 81 | TDRD7 | 8.184846934 | 5.67E−13 | PAPSS2 | −6.291969357 | 3.41E−08 |
| 82 | CNP | 8.178411209 | 5.87E−13 | DNAJB5 | −6.289467072 | 3.44E−08 |
| 83 | BLZF1 | 8.172304201 | 6.06E−13 | PPTC7 | −6.280423452 | 3.62E−08 |
| 84 | KCND1 | 8.154933563 | 6.78E−13 | TSPO | −6.277378996 | 3.67E−08 |
| 85 | TYMS | 8.118631864 | 8.52E−13 | KCNK6 | −6.276050388 | 3.69E−08 |
| 86 | MX2 | 8.117499782 | 8.52E−13 | BRWD3 | −6.262795722 | 3.93E−08 |
| 87 | RUFY4 | 8.116391053 | 8.52E−13 | RAB40C | −6.245902351 | 4.26E−08 |
| 88 | NT5C3A | 8.10794057 | 8.95E−13 | CD1C | −6.23040546 | 4.62E−08 |
| 89 | PARP12 | 8.10523245 | 9.03E−13 | ATP6AP2 | −6.207569059 | 5.19E−08 |
| 90 | SERPING1 | 8.076851829 | 1.09E−12 | ARHGEF40 | −6.198600752 | 5.45E−08 |
| 91 | PARP9 | 8.064017156 | 1.18E−12 | SLC35E1 | −6.195146829 | 5.54E−08 |
| 92 | CYSLTR1 | 8.036041798 | 1.41E−12 | EEF1B2 | −6.188998129 | 5.72E−08 |
| 93 | MT2A | 8.03143347 | 1.44E−12 | HVCN1 | −6.136356366 | 0.000000075 |
| 94 | IFITM1 | 8.024315329 | 1.50E−12 | FGFR1OP | −6.124405872 | 7.96E−08 |
| 95 | BUD31 | 8.015296653 | 1.58E−12 | RFX2 | −6.111353377 | 8.52E−08 |
| 96 | TMEM140 | 8.00242348 | 1.71E−12 | PTRHD1 | −6.101998739 | 8.96E−08 |
| 97 | H2BC5 | 7.980964518 | 1.96E−12 | SLC12A9 | −6.091562743 | 9.48E−08 |
| 98 | IFI35 | 7.977598297 | 1.99E−12 | ENTPD2 | −6.083242685 | 9.82E−08 |
| 99 | GCC1 | 7.943320729 | 2.46E−12 | JADE1 | −6.067673844 | 1.03E−07 |
| 100 | CCDC97 | 7.940124552 | 2.49E−12 | CCNJL | −6.060837055 | 1.07E−07 |

Mid-Control

| | | | | | | |
|---|---|---|---|---|---|---|
| 1 | IFI27 | 16.42258953 | 1.16E−41 | VPS51 | −12.19710925 | 1.11E−18 |
| 2 | EPSTI1 | 15.2261982 | 4.43E−37 | AGTPBP1 | −10.96322333 | 3.87E−17 |
| 3 | LY6E | 14.53615052 | 1.79E−34 | TP53INP2 | −9.842343959 | 1.21E−16 |
| 4 | OAS1 | 13.57059253 | 9.20E−31 | FBL | −9.796585325 | 1.57E−16 |
| 5 | KLHDC7B | 12.73278131 | 1.34E−27 | EIF3K | −9.779197494 | 4.01E−16 |
| 6 | OASL | 12.54902717 | 5.65E−27 | BAG1 | −9.727740659 | 4.49E−16 |
| 7 | ABCA1 | 12.35838359 | 2.58E−26 | FAM168B | −9.468029142 | 4.64E−16 |
| 8 | IFI44L | 12.13600898 | 1.39E−25 | EIF3L | −9.22841139 | 5.39E−16 |
| 9 | SIGLEC1 | 12.12429495 | 1.39E−25 | PDZK1IP1 | −9.224903576 | 8.27E−16 |
| 10 | OTOF | 11.97341251 | 4.68E−25 | AMPD2 | −9.095759231 | 9.46E−16 |
| 11 | OAS3 | 11.89061098 | 8.77E−25 | HADHA | −9.057225768 | 1.55E−15 |
| 12 | IFI44 | 11.73308198 | 3.14E−24 | IGF1R | −8.956849261 | 1.71E−15 |
| 13 | ZCCHC2 | 11.66387499 | 5.26E−24 | NUDT3 | −8.907568279 | 2.06E−15 |
| 14 | OAS2 | 11.41980595 | 3.92E−23 | KLF13 | −8.778140634 | 7.79E−15 |
| 15 | USP18 | 11.37660386 | 5.29E−23 | EMC3 | −8.771259845 | 1.00E−14 |
| 16 | RSAD2 | 11.20287151 | 2.15E−22 | TBC1D14 | −8.645889337 | 1.08E−14 |
| 17 | CMPK2 | 11.06313742 | 6.54E−22 | PRDX5 | −8.531891709 | 1.20E−14 |
| 18 | H4C8 | 11.04073013 | 7.47E−22 | CCNY | −8.520939104 | 1.30E−14 |
| 19 | RRM2 | 10.97632481 | 1.21E−21 | ELOB | −8.519584628 | 1.90E−14 |
| 20 | MKI67 | 10.9353282 | 1.55E−21 | EPB42 | −8.439019015 | 2.18E−14 |
| 21 | SHISA5 | 10.87619448 | 2.42E−21 | ASCC2 | −8.408019474 | 2.86E−14 |
| 22 | TYMS | 10.78213803 | 5.04E−21 | FUNDC2 | −8.359768744 | 4.29E−14 |
| 23 | DDX60 | 10.68499473 | 1.07E−20 | FBXO7 | −8.296890756 | 1.45E−13 |
| 24 | SPATS2L | 10.48812267 | 5.15E−20 | BBOF1 | −8.253050053 | 2.24E−13 |
| 25 | CYSLTR1 | 10.45462578 | 6.51E−20 | FIS1 | −8.163650194 | 2.74E−13 |
| 26 | IL1RN | 10.37605306 | 1.19E−19 | GNAQ | −8.128360712 | 2.74E−13 |
| 27 | REC8 | 10.19358044 | 4.96E−19 | YBX3 | −8.065710677 | 3.12E−13 |
| 28 | AGRN | 10.08100482 | 1.15E−18 | BLVRB | −8.0384885 | 3.49E−13 |
| 29 | TRIM69 | 10.08028636 | 1.15E−18 | OR2W3 | −7.920086558 | 3.61E−13 |
| 30 | IFIT1 | 10.06435566 | 1.26E−18 | ST13 | −7.880109905 | 5.10E−13 |
| 31 | IFIT3 | 9.997174439 | 2.08E−18 | CHMP4B | −7.857612674 | 7.30E−13 |
| 32 | H2BC5 | 9.929373499 | 3.45E−18 | TESC | −7.857211664 | 7.87E−13 |
| 33 | HERC6 | 9.90291575 | 4.05E−18 | AKTIS1 | −7.812650871 | 7.93E−13 |
| 34 | ISG15 | 9.902043067 | 4.05E−18 | KAT8 | −7.769560966 | 1.04E−12 |
| 35 | KIAA1958 | 9.847519889 | 6.04E−18 | SERF2 | −7.70617252 | 1.16E−12 |
| 36 | MX1 | 9.816861864 | 7.29E−18 | SNCA | −7.671390961 | 1.35E−12 |
| 37 | CDC20 | 9.755912296 | 1.09E−17 | TALDO1 | −7.668747127 | 1.35E−12 |
| 38 | CARD6 | 9.752122281 | 1.10E−17 | CAMK1D | −7.651233776 | 1.85E−12 |
| 39 | TRIM5 | 9.736796593 | 1.21E−17 | PTAFR | −7.648496243 | 2.12E−12 |
| 40 | ZBP1 | 9.7243185 | 1.27E−17 | MICAL2 | −7.613822374 | 2.27E−12 |
| 41 | BUB1 | 9.706791959 | 1.43E−17 | SHISA4 | −7.582697075 | 2.41E−12 |
| 42 | GALM | 9.686940646 | 1.63E−17 | CCNJL | −7.580119067 | 2.42E−12 |
| 43 | CEACAM1 | 9.649037027 | 2.15E−17 | TTC25 | −7.571252795 | 2.55E−12 |
| 44 | AKIRIN2 | 9.615515544 | 2.73E−17 | FUZ | −7.525983382 | 3.18E−12 |
| 45 | HERC5 | 9.538286096 | 4.81E−17 | OPTN | −7.497627123 | 4.14E−12 |
| 46 | ZFYVE26 | 9.537110866 | 4.81E−17 | TMOD1 | −7.48991928 | 4.17E−12 |
| 47 | IFI6 | 9.531655712 | 4.92E−17 | INPP5K | −7.476967879 | 4.19E−12 |
| 48 | MYLIP | 9.519570033 | 5.30E−17 | OAT | −7.474105787 | 5.21E−12 |
| 49 | XAF1 | 9.466172057 | 7.71E−17 | GYPC | −7.473835433 | 5.63E−12 |
| 50 | ZBTB32 | 9.42179856 | 1.06E−16 | SLC8B1 | −7.458274632 | 7.23E−12 |
| 51 | TK1 | 9.418361241 | 1.07E−16 | UNC119B | −7.444961077 | 1.23E−11 |
| 52 | KLHDC8B | 9.40025041 | 1.21E−16 | STRADB | −7.443139646 | 1.43E−11 |
| 53 | JCHAIN | 9.383271558 | 1.36E−16 | SLC4A1 | −7.434440482 | 1.44E−11 |
| 54 | IFIH1 | 9.285558253 | 2.81E−16 | RFLNB | −7.423831771 | 1.47E−11 |
| 55 | EPHB2 | 9.261548532 | 3.32E−16 | PPM1B | −7.407186171 | 1.51E−11 |
| 56 | CD300A | 9.186515098 | 5.58E−16 | TSPAN5 | −7.393346391 | 1.62E−11 |
| 57 | CD38 | 9.155468696 | 6.94E−16 | RELL1 | −7.381350332 | 1.63E−11 |
| 58 | TPX2 | 9.152360509 | 7.00E−16 | BRI3BP | −7.365103915 | 2.21E−11 |
| 59 | RAB8A | 9.138567851 | 7.65E−16 | C12orf10 | −7.347142543 | 2.24E−11 |
| 60 | HES4 | 9.112201308 | 9.19E−16 | RXRA | −7.338198066 | 2.36E−11 |
| 61 | KCND1 | 9.105575377 | 9.52E−16 | CCDC125 | −7.319639395 | 2.62E−11 |
| 62 | RTP4 | 9.077823193 | 1.14E−15 | LGALS3 | −7.318063703 | 3.27E−11 |
| 63 | SERPING1 | 9.061541463 | 1.26E−15 | FAM210B | −7.284524212 | 3.80E−11 |
| 64 | SP100 | 9.059474824 | 1.26E−15 | FBXO9 | −7.277610409 | 4.03E−11 |
| 65 | CDT1 | 9.057595077 | 1.26E−15 | MXI1 | −7.268444581 | 4.25E−11 |
| 66 | IFIT5 | 9.044290726 | 1.37E−15 | STK11IP | −7.26457108 | 4.26E−11 |
| 67 | EIF2AK2 | 9.025210073 | 1.56E−15 | MAPK1 | −7.259142453 | 4.37E−11 |
| 68 | CENPF | 9.015621137 | 1.65E−15 | SELENBP1 | −7.236113052 | 4.54E−11 |
| 69 | KIFC1 | 9.007543142 | 1.73E−15 | EIF3H | −7.231062804 | 5.85E−11 |
| 70 | TMX2 | 8.932689093 | 2.95E−15 | PSMF1 | −7.218503503 | 6.28E−11 |
| 71 | SP140 | 8.87782098 | 4.32E−15 | MPP1 | −7.192276978 | 6.31E−11 |
| 72 | BLZF1 | 8.862201329 | 4.79E−15 | UXT | −7.184391985 | 6.45E−11 |
| 73 | ABCG1 | 8.84150084 | 5.52E−15 | WDR45 | −7.157759717 | 6.69E−11 |
| 74 | LMO2 | 8.826031254 | 6.11E−15 | TMCO3 | −7.153228652 | 6.89E−11 |
| 75 | NTNG2 | 8.789056941 | 7.93E−15 | AHCYL1 | −7.147270798 | 7.14E−11 |
| 76 | MAD2L1BP | 8.783872425 | 8.14E−15 | AZIN1 | −7.107875848 | 8.72E−11 |
| 77 | PKMYT1 | 8.659517753 | 1.95E−14 | SPATA6 | −7.095533165 | 8.91E−11 |
| 78 | CDC42EP3 | 8.644761757 | 2.13E−14 | YBX1 | −7.092243448 | 1.02E−10 |
| 79 | KIAA0895L | 8.589990725 | 3.13E−14 | GMCL1 | −7.088442585 | 1.02E−10 |
| 80 | IRF7 | 8.57192831 | 3.53E−14 | FOXO1 | −7.04788359 | 1.26E−10 |
| 81 | C2CD3 | 8.548996543 | 4.12E−14 | UBXN6 | −7.003751477 | 1.28E−10 |
| 82 | NUSAP1 | 8.466301895 | 7.18E−14 | CCDC124 | −6.97927242 | 1.29E−10 |
| 83 | TDRD7 | 8.452731024 | 7.83E−14 | HAGH | −6.967611373 | 1.35E−10 |
| 84 | POU2AF1 | 8.439040157 | 8.48E−14 | BRD1 | −6.964351184 | 1.38E−10 |
| 85 | HELZ2 | 8.414594415 | 1.00E−13 | XRN2 | −6.960118658 | 1.38E−10 |
| 86 | SCO2 | 8.395468645 | 1.12E−13 | TCP11L2 | −6.943136786 | 1.44E−10 |
| 87 | IFIT2 | 8.370484515 | 1.33E−13 | RPGR | −6.938863075 | 1.67E−10 |
| 88 | BATF2 | 8.367058786 | 1.35E−13 | BTF3 | −6.910388853 | 1.70E−10 |
| 89 | IFITM1 | 8.335204841 | 1.65E−13 | SLC25A37 | −6.910126865 | 1.88E−10 |
| 90 | TXNDC5 | 8.333709016 | 1.65E−13 | TPGS2 | −6.878880052 | 1.93E−10 |
| 91 | LRFN1 | 8.333471803 | 1.65E−13 | TBC1D17 | −6.865887485 | 1.94E−10 |
| 92 | TRIM22 | 8.284361654 | 2.30E−13 | PPM1F | −6.857428785 | 2.00E−10 |
| 93 | TMEM140 | 8.264152741 | 2.63E−13 | PTMS | −6.852430716 | 2.11E−10 |
| 94 | MOV10 | 8.256600233 | 2.75E−13 | WDR13 | −6.849142264 | 2.37E−10 |
| 95 | DDAH2 | 8.238158524 | 3.08E−13 | PGK1 | −6.846081905 | 2.42E−10 |
| 96 | IQSEC1 | 8.232920719 | 3.17E−13 | HBB | −6.841274504 | 3.09E−10 |
| 97 | NEXN | 8.230288451 | 3.20E−13 | TAF1 | −6.833105453 | 3.70E−10 |
| 98 | MCM4 | 8.217238786 | 3.48E−13 | CDC123 | −6.827189899 | 3.76E−10 |
| 99 | MRPS18B | 8.193317723 | 4.06E−13 | SLC25A39 | −6.823097065 | 3.78E−10 |
| 100 | IGLL5 | 8.193040451 | 4.06E−13 | SPSB3 | −6.798774057 | 3.94E−10 |

Early Post-Control

| | | | | | | |
|---|---|---|---|---|---|---|
| 1 | EPSTI1 | 7.68E+00 | 1.69E−09 | KLF13 | −6.63E+00 | 4.59E−07 |
| 2 | ZBTB32 | 7.45E+00 | 3.86E−09 | VPS51 | −6.57E+00 | 5.07E−07 |
| 3 | IFI27 | 6.37E+00 | 1.06E−06 | CD1D | −6.49E+00 | 6.29E−07 |
| 4 | ABCA1 | 6.19E+00 | 2.45E−06 | PDK4 | −6.18E+00 | 2.45E−06 |
| 5 | INSL3 | 5.96E+00 | 6.05E−06 | TP53INP2 | −6.10E+00 | 3.39E−06 |
| 6 | MYLIP | 5.77E+00 | 1.46E−05 | RCSD1 | −6.06E+00 | 3.82E−06 |
| 7 | CDC42EP3 | 5.72E+00 | 1.67E−05 | BRD1 | −5.84E+00 | 1.11E−05 |
| 8 | MKI67 | 5.64E+00 | 2.49E−05 | ARFRP1 | −5.75E+00 | 1.58E−05 |
| 9 | ELMO2 | 5.57E+00 | 3.32E−05 | FBL | −5.42E+00 | 5.71E−05 |
| 10 | PLA2G15 | 5.53E+00 | 3.74E−05 | NUDT3 | −5.20E+00 | 1.21E−04 |
| 11 | TNFRSF13B | 5.53E+00 | 3.74E−05 | CDC37 | −5.19E+00 | 1.28E−04 |
| 12 | PHF21A | 5.44E+00 | 5.57E−05 | PRDX5 | −4.99E+00 | 2.69E−04 |
| 13 | SLC1A4 | 5.43E+00 | 5.71E−05 | EDF1 | −4.97E+00 | 2.99E−04 |
| 14 | TYMS | 5.35E+00 | 7.42E−05 | KLF9 | −4.95E+00 | 3.07E−04 |
| 15 | TK1 | 5.35E+00 | 7.42E−05 | SECISBP2L | −4.93E+00 | 3.34E−04 |
| 16 | RRM2 | 5.34E+00 | 7.48E−05 | SLMAP | −4.89E+00 | 3.83E−04 |
| 17 | ADRB2 | 5.28E+00 | 9.62E−05 | TAF1 | −4.81E+00 | 5.00E−04 |
| 18 | CD180 | 5.27E+00 | 9.62E−05 | PTPN11 | −4.79E+00 | 5.14E−04 |
| 19 | TPX2 | 5.27E+00 | 9.62E−05 | CCDC85B | −4.72E+00 | 6.62E−04 |
| 20 | TOP2A | 5.26E+00 | 9.91E−05 | VSIR | −4.70E+00 | 6.94E−04 |
| 21 | PRDM1 | 5.21E+00 | 1.21E−04 | PDE3B | −4.70E+00 | 6.98E−04 |
| 22 | IL15RA | 5.16E+00 | 1.39E−04 | CARS2 | −4.6619028 | 0.000761705 |
| 23 | CDCA7 | 5.16E+00 | 1.39E−04 | MYCL | −4.645214311 | 0.000800037 |
| 24 | OASL | 5.10E+00 | 1.76E−04 | DUS1L | −4.63E+00 | 8.23E−04 |
| 25 | CARD6 | 5.06E+00 | 2.09E−04 | PPM1B | −4.62E+00 | 8.23E−04 |
| 26 | ICOS | 5.02E+00 | 2.49E−04 | SREK1 | −4.59E+00 | 8.92E−04 |
| 27 | CDC20 | 5.02E+00 | 2.49E−04 | ZBTB14 | −4.55E+00 | 1.04E−03 |
| 28 | JCHAIN | 4.96E+00 | 3.05E−04 | ACADSB | −4.49E+00 | 1.24E−03 |
| 29 | C2CD3 | 4.92E+00 | 3.39E−04 | PRKAR1A | −4.49E+00 | 1.25E−03 |
| 30 | STIL | 4.83E+00 | 4.74E−04 | MBNL1 | −4.47E+00 | 1.30E−03 |
| 31 | P2RX4 | 4.83E+00 | 4.74E−04 | SLC26A2 | −4.47E+00 | 1.30E−03 |
| 32 | NUSAP1 | 4.83E+00 | 4.74E−04 | LY75 | −4.47E+00 | 1.30E−03 |
| 33 | SPATS2 | 4.83E+00 | 4.74E−04 | MYBL1 | −4.47E+00 | 1.31E−03 |
| 34 | POU2AF1 | 4.81E+00 | 5.00E−04 | TIMM13 | −4.46E+00 | 1.32E−03 |
| 35 | PKMYT1 | 4.79E+00 | 5.14E−04 | SDE2 | −4.45E+00 | 1.35E−03 |
| 36 | OTOF | 4.78E+00 | 5.37E−04 | MZT2B | −4.44E+00 | 1.38E−03 |
| 37 | PHF19 | 4.77E+00 | 5.43E−04 | INIP | −4.425551566 | 0.001442138 |
| 38 | LY6E | 4.74E+00 | 6.32E−04 | OAT | −4.413077346 | 0.001465652 |
| 39 | PTPRO | 4.72E+00 | 6.62E−04 | TRMT13 | −4.41E+00 | 1.47E−03 |
| 40 | KIF11 | 4.71E+00 | 6.70E−04 | TSPAN13 | −4.40E+00 | 1.53E−03 |
| 41 | LETM2 | 4.69E+00 | 7.06E−04 | DENND6A | −4.39E+00 | 1.57E−03 |
| 42 | CARMIL3 | 4.68E+00 | 7.49E−04 | SNRK | −4.39E+00 | 1.57E−03 |
| 43 | KIFC1 | 4.67E+00 | 7.56E−04 | WRNIP1 | −4.39E+00 | 1.57E−03 |
| 44 | NT5DC2 | 4.67E+00 | 7.59E−04 | FOXO1 | −4.37E+00 | 1.67E−03 |
| 45 | CDT1 | 4.66E+00 | 7.62E−04 | IRF8 | −4.35E+00 | 1.78E−03 |
| 46 | FAM222B | 4.634841068 | 0.0008233 | HNRNPA0 | −4.334053654 | 0.001870835 |
| 47 | PTTG1 | 4.631845534 | 0.0008233 | PIK3R1 | −4.31E+00 | 2.03E−03 |
| 48 | ABCG1 | 4.62E+00 | 8.23E−04 | KCTD20 | −4.30E+00 | 2.14E−03 |
| 49 | CES4A | 4.62E+00 | 8.23E−04 | HNRNPM | −4.28E+00 | 2.28E−03 |
| 50 | IRF4 | 4.62E+00 | 8.23E−04 | INO80B | −4.28E+00 | 2.28E−03 |
| 51 | RNF175 | 4.61E+00 | 8.23E−04 | ESRRA | −4.27409623 | 0.002301458 |
| 52 | RNF185 | 4.61E+00 | 8.35E−04 | EIF3K | −4.27E+00 | 2.30E−03 |
| 53 | FGFR1 | 4.56E+00 | 9.92E−04 | STUB1 | −4.26E+00 | 2.39E−03 |
| 54 | OAS1 | 4.55E+00 | 1.04E−03 | KIAA0513 | −4.243279999 | 0.002476344 |
| 55 | C1QA | 4.55E+00 | 1.04E−03 | MEX3C | −4.241984038 | 0.002476344 |
| 56 | SMARCD3 | 4.54E+00 | 1.07E−03 | SLC30A5 | −4.19E+00 | 2.96E−03 |
| 57 | IGLL5 | 4.53E+00 | 1.08E−03 | HMGB1 | −4.19E+00 | 2.96E−03 |
| 58 | IFI44L | 4.50E+00 | 1.22E−03 | LSM14B | −4.18E+00 | 2.97E−03 |
| 59 | C7orf61 | 4.50035731 | 0.0012164 | LBR | −4.18E+00 | 2.97E−03 |
| 60 | LPP | 4.47E+00 | 1.32E−03 | YTHDC2 | −4.167598504 | 0.002989043 |
| 61 | CENPF | 4.45E+00 | 1.38E−03 | TUBA4A | −4.17E+00 | 2.99E−03 |
| 62 | AP3M2 | 4.44E+00 | 1.39E−03 | NR1D2 | −4.15E+00 | 3.10E−03 |
| 63 | IQSEC1 | 4.44E+00 | 1.39E−03 | ZSCAN25 | −4.15E+00 | 3.12E−03 |
| 64 | ST3GAL6 | 4.43E+00 | 1.43E−03 | AZIN1 | −4.12E+00 | 3.37E−03 |
| 65 | KLHDC7B | 4.42E+00 | 1.47E−03 | TALDO1 | −4.11E+00 | 3.46E−03 |
| 66 | FANCI | 4.42E+00 | 1.47E−03 | YTHDF1 | −4.10E+00 | 3.65E−03 |
| 67 | TRAM2 | 4.41E+00 | 1.48E−03 | B3GALT6 | −4.09E+00 | 3.69E−03 |
| 68 | TXNDC5 | 4.37E+00 | 1.69E−03 | CREBZF | −4.09E+00 | 3.72E−03 |
| 69 | RRAS | 4.36E+00 | 1.73E−03 | ARL6IP4 | −4.09E+00 | 3.72E−03 |
| 70 | OR52K1 | 4.335778479 | 0.0018708 | METTL1 | −4.08E+00 | 3.75E−03 |
| 71 | SERPING1 | 4.310627432 | 0.0020346 | LFNG | −4.08E+00 | 3.77E−03 |
| 72 | CERCAM | 4.27E+00 | 2.30E−03 | TSN | −4.07E+00 | 3.93E−03 |
| 73 | RHOBTB2 | 4.253693817 | 0.0024309 | CCNT2 | −4.06E+00 | 4.02E−03 |
| 74 | GPT2 | 4.25E+00 | 2.46E−03 | SNX18 | −4.05E+00 | 4.06E−03 |
| 75 | TLR7 | 4.25E+00 | 2.46E−03 | AAMP | −4.04E+00 | 4.16E−03 |
| 76 | CBFA2T3 | 4.22E+00 | 2.65E−03 | DTX4 | −4.04E+00 | 4.24E−03 |
| 77 | SLC22A1 | 4.22E+00 | 2.65E−03 | CCNJL | −4.03E+00 | 4.27E−03 |
| 78 | MAMDC4 | 4.214715964 | 0.0027167 | TSSC4 | −4.02E+00 | 4.40E−03 |
| 79 | PRDX4 | 4.21E+00 | 2.78E−03 | LATS1 | −4.02E+00 | 4.41E−03 |
| 80 | SLC9A9 | 4.19E+00 | 2.91E−03 | EEF1D | −4.01E+00 | 4.55E−03 |
| 81 | LIMK1 | 4.19E+00 | 2.91E−03 | TSR3 | −4.00E+00 | 4.71E−03 |
| 82 | HDAC5 | 4.18E+00 | 2.96E−03 | ZNF304 | −4.00E+00 | 4.81E−03 |
| 83 | PROK2 | 4.18E+00 | 2.97E−03 | IRS2 | −3.99E+00 | 4.86E−03 |
| 84 | TAS1R3 | 4.18E+00 | 2.97E−03 | NDUFV1 | −3.98E+00 | 5.13E−03 |
| 85 | C1QB | 4.17E+00 | 2.97E−03 | CCDC186 | −3.97E+00 | 5.13E−03 |
| 86 | TLR5 | 4.17E+00 | 2.98E−03 | RBM3 | −3.96E+00 | 5.24E−03 |
| 87 | ADAP2 | 4.17E+00 | 2.99E−03 | ATP5F1D | −3.96E+00 | 5.24E−03 |
| 88 | ALPK1 | 4.16E+00 | 3.08E−03 | SLC25A6 | −3.96E+00 | 5.24E−03 |
| 89 | GLI1 | 4.16E+00 | 3.09E−03 | SIVA1 | −3.96E+00 | 5.28E−03 |
| 90 | TNFRSF17 | 4.15E+00 | 3.14E−03 | HSD17B10 | −3.96E+00 | 5.29E−03 |
| 91 | ATP6V0A1 | 4.15E+00 | 3.14E−03 | RANBP6 | −3.95E+00 | 5.34E−03 |
| 92 | FCGR1B | 4.14E+00 | 3.19E−03 | CNOT6L | −3.950707002 | 0.005353505 |
| 93 | EZH2 | 4.14E+00 | 3.22E−03 | BORCS6 | −3.94E+00 | 5.48E−03 |
| 94 | AIM2 | 4.12E+00 | 3.44E−03 | RP2 | −3.94E+00 | 5.48E−03 |
| 95 | HCAR3 | 4.105741167 | 0.0035607 | GSTP1 | −3.932277267 | 0.005594995 |
| 96 | LRRC37B | 4.09E+00 | 3.70E−03 | TBL3 | −3.925500074 | 0.00569298 |
| 97 | COA6 | 4.08E+00 | 3.77E−03 | RHOU | −3.92E+00 | 5.74E−03 |
| 98 | AMN1 | 4.07E+00 | 3.92E−03 | ZNF146 | −3.92E+00 | 5.83E−03 |
| 99 | ZCCHC2 | 4.07E+00 | 3.93E−03 | ZNF518A | −3.91E+00 | 6.07E−03 |
| 100 | CENPE | 4.05E+00 | 4.11E−03 | F2R | −3.90E+00 | 6.13E−03 |

Late-Post Control

| | | | | | | |
|---|---|---|---|---|---|---|
| 1 | ZBTB32 | 6.38880158 | 4.07E−06 | PDK4 | −6.332740509 | 4.07E−06 |
| 2 | ELMO2 | 4.968402325 | 1.70E−03 | TP53INP2 | −5.221020733 | 9.84E−04 |
| 3 | MYLIP | 4.89560255 | 2.11E−03 | TBL3 | −5.189479444 | 9.84E−04 |
| 4 | POU2AF1 | 4.741432274 | 3.49E−03 | PDE3B | −5.148865483 | 0.000984423 |
| 5 | TNFRSF17 | 4.709414871 | 3.68E−03 | POLR3H | −5.10014908 | 1.04E−03 |
| 6 | SREBF1 | 4.613606272 | 0.0046504 | ACADVL | −4.799879365 | 2.95E−03 |
| 7 | DENND5B | 4.586917372 | 0.0046504 | PRPF31 | −4.646201753 | 4.51E−03 |
| 8 | JCHAIN | 4.585252602 | 0.0046504 | CDC37 | −4.44739053 | 0.005314862 |
| 9 | FCGR1B | 4.576301857 | 0.0046504 | CD1D | −4.425860005 | 0.005594563 |
| 10 | NUSAP1 | 4.543404149 | 0.0050758 | WRNIP1 | −4.38257516 | 0.006278749 |
| 11 | C1QA | 4.518702892 | 0.0051001 | BRD1 | −4.368426969 | 0.006447451 |
| 12 | IRF4 | 4.517507359 | 0.0051001 | SPIN1 | −4.354504103 | 0.006621017 |
| 13 | CD59 | 4.485451916 | 0.0053149 | RCSD1 | −4.325828831 | 0.007031018 |
| 14 | TYMS | 4.457080805 | 0.0053149 | ENOPH1 | −4.319396729 | 0.007031018 |
| 15 | SLC1A4 | 4.456954947 | 0.0053149 | KLF13 | −4.318555437 | 0.007031018 |
| 16 | FCGR1A | 4.447288091 | 0.0053149 | DDX54 | −4.236108305 | 0.00805004 |
| 17 | AIM2 | 4.446414132 | 0.0053149 | IRS2 | −4.222153324 | 0.008336794 |
| 18 | AP3M2 | 4.393606442 | 0.0062052 | SDE2 | −4.177426603 | 0.009607086 |
| 19 | ZWINT | 4.310927649 | 0.0070520 | KLF9 | −4.157040844 | 0.010230028 |
| 20 | SCARF1 | 4.279208934 | 0.0078488 | RELL1 | −4.143609556 | 0.010584194 |
| 21 | TK1 | 4.268400709 | 0.0079914 | ST14 | −4.129319827 | 0.010993301 |
| 22 | TOP2A | 4.257845278 | 0.0080500 | STK26 | −4.117932811 | 0.011054653 |
| 23 | CDC20 | 4.250276958 | 0.0080500 | KCTD18 | −4.091935874 | 0.012062417 |
| 24 | RRM2 | 4.238242741 | 0.0080500 | FAM50A | −4.076920631 | 0.012337933 |
| 25 | FANCI | 4.236566225 | 0.0080500 | PRDX5 | −4.069708369 | 0.012469064 |
| 26 | TXNDC5 | 4.210547755 | 0.0085521 | SMAP2 | −4.01422486 | 0.014799941 |
| 27 | RRAS | 4.118958933 | 0.0110547 | TRAPPC8 | −3.97662044 | 0.016675447 |
| 28 | PRDM1 | 4.082785853 | 0.0122801 | ZCCHC14 | −3.969218882 | 0.016675447 |
| 29 | MKI67 | 4.058958931 | 0.0127895 | IREB2 | −3.949975169 | 0.017045143 |
| 30 | ALPK1 | 4.050701072 | 0.0129885 | B3GALT6 | −3.93780378 | 0.017344867 |
| 31 | MSS51 | 3.968813905 | 0.0166754 | HMOX2 | −3.86192462 | 0.020038996 |
| 32 | CDCA7 | 3.966137159 | 0.0166754 | PHB | −3.849871233 | 0.02073738 |
| 33 | AKAP9 | 3.96348685 | 0.0166754 | FKBP5 | −3.819745304 | 0.022458047 |
| 34 | TLR5 | 3.955933251 | 0.0169114 | IL2RB | −3.809855648 | 0.023047418 |
| 35 | FKBP11 | 3.940995542 | 0.0173449 | ARFRP1 | −3.804939762 | 0.023205466 |
| 36 | ZBTB7A | 3.931260411 | 0.0175342 | GZMB | −3.797826011 | 0.023276014 |
| 37 | EFEMP2 | 3.916384528 | 0.0183268 | ZMYND11 | −3.78209244 | 0.023624755 |
| 38 | RECQL4 | 3.892947896 | 0.0194600 | CCNJL | −3.747334908 | 0.025600293 |
| 39 | NFKBIZ | 3.889151188 | 0.0194600 | ZBTB9 | −3.744093095 | 0.025600293 |
| 40 | INTS8 | 3.887993111 | 0.0194600 | MEGF9 | −3.731681177 | 0.025666802 |
| 41 | PEX26 | 3.886625835 | 0.0194600 | KRT10 | −3.720658427 | 0.025803952 |
| 42 | SPATS2 | 3.875168557 | 0.0195877 | PPM1B | −3.694717566 | 0.027575939 |
| 43 | GYG1 | 3.872034651 | 0.0195877 | TMEM141 | −3.681302811 | 0.027990148 |
| 44 | TMEM156 | 3.871800906 | 0.0195877 | ZSCAN25 | −3.669924287 | 0.027990148 |
| 45 | TNFRSF13B | 3.871077929 | 0.0195877 | CIPC | −3.666700169 | 0.028081924 |
| 46 | MFF | 3.831069981 | 0.0220347 | ZNF57 | −3.657028021 | 0.028760254 |
| 47 | NT5DC2 | 3.819702384 | 0.0224580 | ZCCHC10 | −3.655104081 | 0.028760254 |
| 48 | CARD6 | 3.79908748 | 0.0232760 | TIGD3 | −3.653513183 | 0.028760254 |
| 49 | IGLL5 | 3.794896518 | 0.0232760 | SLMAP | −3.639437253 | 0.029631371 |
| 50 | DYSF | 3.78468207 | 0.0236248 | CACNB4 | −3.635261048 | 0.029631371 |
| 51 | ITGA7 | 3.782719272 | 0.0236248 | INAFM2 | −3.634301472 | 0.029631371 |
| 52 | CACNA1E | 3.770401617 | 0.0242805 | NEK7 | −3.62791604 | 0.029882307 |
| 53 | CASP5 | 3.769176854 | 0.0242805 | MRPL28 | −3.591477208 | 0.033681979 |
| 54 | CST7 | 3.756978671 | 0.0251681 | PHC2 | −3.57986676 | 0.034487208 |
| 55 | NCOA3 | 3.745342836 | 0.0256003 | MAP2K1 | −3.578883461 | 0.034487208 |
| 56 | CARD16 | 3.738678286 | 0.0256668 | DAPK1 | −3.578781114 | 0.034487208 |
| 57 | KMT5A | 3.736255502 | 0.0256668 | BEX4 | −3.561491676 | 0.036200391 |
| 58 | PIM3 | 3.732256408 | 0.0256668 | MATK | −3.55868674 | 0.036300852 |
| 59 | GBP2 | 3.729012752 | 0.0256668 | DAAM2 | −3.547569941 | 0.037253488 |
| 60 | CLEC1A | 3.727243623 | 0.0256668 | HIGD1A | −3.539811178 | 0.037681359 |
| 61 | CCDC88A | 3.722158288 | 0.0258040 | PTPRS | −3.536482311 | 0.037681359 |
| 62 | SETD5 | 3.708796815 | 0.0267343 | PUF60 | −3.528959085 | 0.037963146 |
| 63 | COBLL1 | 3.705414228 | 0.0268200 | SPATA6 | −3.526738265 | 0.037963146 |
| 64 | SEL1L3 | 3.693073079 | 0.0275759 | PLVAP | −3.526680803 | 0.037963146 |
| 65 | KIF11 | 3.689988543 | 0.0276394 | EDF1 | −3.513682828 | 0.038833225 |
| 66 | CD274 | 3.675302918 | 0.0279901 | FAM174A | −3.51103056 | 0.038833225 |
| 67 | VPS9D1 | 3.673309815 | 0.0279901 | IRF8 | −3.491538835 | 0.040822801 |
| 68 | UBE2N | 3.670899136 | 0.0279901 | SEC23A | −3.484781623 | 0.041219278 |
| 69 | BUB1 | 3.6707222 | 0.0279901 | PPIL4 | −3.48362355 | 0.041219278 |
| 70 | NARF | 3.669885325 | 0.0279901 | DNAJA3 | −3.467846493 | 0.042867248 |
| 71 | CENPE | 3.645454935 | 0.0293944 | TRMT61A | −3.460172131 | 0.043659048 |
| 72 | CES4A | 3.633119938 | 0.0296314 | WDR74 | −3.458799793 | 0.043659048 |
| 73 | SLC26A8 | 3.63232124 | 0.0296314 | CYP1B1 | −3.454706216 | 0.043838561 |
| 74 | GNRH1 | 3.624704593 | 0.0300028 | RABL6 | −3.454036518 | 0.043838561 |
| 75 | PRR11 | 3.567297193 | 0.0357052 | OAF | −3.448699387 | 0.043990459 |
| 76 | GPR84 | 3.548044205 | 0.0372534 | ERGIC1 | −3.442376 | 0.044623845 |
| 77 | TPX2 | 3.542572508 | 0.0376633 | TSR3 | −3.435363055 | 0.045481532 |
| 78 | HCN3 | 3.537680142 | 0.0376813 | WDR61 | −3.416531836 | 0.047776566 |
| 79 | USB1 | 3.529913407 | 0.0379631 | SMAD7 | −3.408652537 | 0.048844357 |
| 80 | PHF21A | 3.518055943 | 0.0388332 | LAMTOR4 | −3.406988746 | 0.048846375 |
| 81 | ZBTB32 | 6.38880158 | 4.07E−06 | PDK4 | −6.332740509 | 4.07E−06 |
| 82 | ELMO2 | 4.968402325 | 1.70E−03 | TP53INP2 | −5.221020733 | 9.84E−04 |
| 83 | MYLIP | 4.89560255 | 2.11E−03 | TBL3 | −5.189479444 | 9.84E−04 |
| 84 | C7orf61 | 3.514290916 | 0.0388332 | | | |
| 85 | ZC3H12D | 3.51122884 | 0.0388332 | | | |
| 86 | GABBR1 | 3.495728338 | 0.040776 | | | |
| 87 | AQP3 | 3.492348847 | 0.0408228 | | | |
| 88 | C1QB | 3.48991015 | 0.0408228 | | | |
| 89 | PARM1 | 3.472069985 | 0.0426972 | | | |
| 90 | TIFA | 3.467407705 | 0.0428672 | | | |
| 91 | CDT1 | 3.452450928 | 0.0438386 | | | |
| 92 | GLRX | 3.448064598 | 0.0439905 | | | |
| 93 | FBXW7 | 3.432589275 | 0.0456594 | | | |
| 94 | GOLGA1 | 3.430824536 | 0.0456744 | | | |
| 95 | PCYT1A | 3.401185216 | 0.0495724 | | | |

TABLE 2.3
(Top 100 DMS detected over time relative to pre−infection Control.
Raw data; FDR < 0.05. Abbreviations: Hypo, hypomethylated
CpG sites; Hyper, hypermethylated CpG sites;
Raw—No correction for cell type proportions, FDR < 0.05)
First-Control

| Hypo methylated | | | | | Hyper methylated | | | |
|---|---|---|---|---|---|---|---|---|
| Rank | CpG | Gene | T | Adj. P. Val | CpG | Gene | T | Adj. P. Val. |
| 1 | cg02650017 | PHOSPHO1 | −8.333921723 | 5.53E−11 | cg11900509 | ANXA11 | 5.253760753 | 0.00430567 |
| 2 | cg07878065 | | −7.788403325 | 2.40E−09 | cg12099754 | TRPM3 | 4.806221725 | 0.030221879 |
| 3 | cg10161996 | | −7.479550341 | 1.76E−08 | cg16550184 | CLEC4A | 4.688167714 | 0.04874817 |
| 4 | cg02138684 | | −7.183650921 | 1.16E−07 | | | | |
| 5 | cg13504881 | | −7.157516356 | 1.16E−07 | | | | |
| 6 | cg24002003 | | −7.066654917 | 1.69E−07 | | | | |
| 7 | cg13575798 | SND1 | −7.059736905 | 1.69E−07 | | | | |
| 8 | cg26416615 | ARID5B | −6.748457376 | 1.32E−06 | | | | |
| 9 | cg21587506 | IFIT3 | −6.554146735 | 4.40E−06 | | | | |
| 10 | cg17114584 | IRF7 | −6.354748453 | 1.48E−05 | | | | |
| 11 | cg08539067 | GPX1 | −6.331520855 | 1.56E−05 | | | | |
| 12 | cg03699074 | FAM38A | −6.252241713 | 0.00002385 | | | | |
| 13 | cg00607627 | LAT | −6.155472481 | 4.07E−05 | | | | |
| 14 | cg26562462 | TBC1D14 | −5.872489938 | 0.000216906 | | | | |
| 15 | cg25739715 | OSM | −5.759782391 | 0.000397172 | | | | |
| 16 | cg25114611 | FKBP5 | −5.745999258 | 0.000403991 | | | | |
| 17 | cg03753191 | EPSTI1 | −5.604394069 | 0.000869575 | | | | |
| 18 | cg10636246 | AIM2 | −5.562282595 | 0.001046379 | | | | |
| 19 | cg13421019 | PPFIBP2 | −5.504338316 | 0.001379569 | | | | |
| 20 | cg05316065 | GSDMC | −5.460283517 | 0.00167466 | | | | |
| 21 | cg23017826 | ZDHHC20 | −5.452319774 | 0.00167466 | | | | |
| 22 | cg00490406 | AIM2 | −5.411537665 | 0.002009095 | | | | |
| 23 | cg04864378 | | −5.364168252 | 0.002501079 | | | | |
| 24 | cg04725636 | DNAJC5B | −5.249909931 | 0.00430567 | | | | |
| 25 | cg16315329 | CISH | −5.241659692 | 0.00432956 | | | | |
| 26 | cg02276017 | PSMA1 | −5.188205905 | 0.005562737 | | | | |
| 27 | cg25563198 | FKBP5 | −5.085624216 | 0.009256808 | | | | |
| 28 | cg20692268 | | −4.996197173 | 0.014155393 | | | | |
| 29 | cg06135068 | | −4.991105709 | 0.014155393 | | | | |
| 30 | cg05304729 | MNDA | −4.955086923 | 0.016497028 | | | | |
| 31 | cg11170544 | TTC22 | −4.898501976 | 0.021346855 | | | | |
| 32 | cg12159504 | STAT1 | −4.878776529 | 0.022589723 | | | | |
| 33 | cg21806732 | SEPT5 | −4.875415507 | 0.022589723 | | | | |
| 34 | cg06703222 | NFAT5 | −4.845126978 | 0.025572271 | | | | |
| 35 | cg00902153 | LPP | −4.773096985 | 0.034682571 | | | | |
| 36 | cg04260633 | TTYH3 | −4.758307894 | 0.036340235 | | | | |
| 37 | cg16462073 | SPNS1 | −4.719956475 | 0.042785405 | | | | |

Mid-Control

| Hypo Methylated | | | | | Hypermethylated | | | |
|---|---|---|---|---|---|---|---|---|
| Rank | CpG | Gene | T | Adj. P. Val | CpG | Gene | T | Adj. P Val |
| 1 | cg22930808 | PARP9 | −12.04966149 | 1.38E−27 | cg12877361 | OAS1 | 11.79538341 | 1.46E−26 |
| 2 | cg21549285 | MX1 | −11.44044201 | 6.19E−25 | cg06146977 | EPSTI1 | 10.43012053 | 1.83E−20 |
| 3 | cg17114584 | IRF7 | −11.34121565 | 1.45E−24 | cg19020860 | TMPRSS2 | 9.279439765 | 9.27E−16 |
| 4 | cg10549986 | RSAD2 | −11.22345075 | 4.43E−24 | cg03100203 | NRIR | 9.178940679 | 1.92E−15 |
| 5 | cg23299102 | | −10.49859121 | 1.03E−20 | cg06217905 | OAS2 | 7.914735022 | 6.49E−11 |
| 6 | cg03753191 | EPSTI1 | −10.17478103 | 2.27E−19 | cg04315689 | DGUOK-AS1 | 7.85790642 | 9.43E−11 |
| 7 | cg10161996 | | −9.898165818 | 3.33E−18 | cg04708790 | OAS1 | 7.854913571 | 9.43E−11 |
| 8 | cg07878065 | | −9.669313014 | 2.88E−17 | cg24908179 | DDX60 | 7.799745067 | 1.37E−10 |
| 9 | cg22862003 | MX1 | −9.492244313 | 1.38E−16 | cg02183564 | CCDC146 | 7.792789714 | 1.40E−10 |
| 10 | cg11251971 | CYSTM1 | −9.488557384 | 1.38E−16 | cg09063556 | CMTM4 | 7.770214617 | 1.63E−10 |
| 11 | cg24002003 | | −9.269271038 | 9.47E−16 | cg03247739 | CREBBP | 7.564218092 | 7.16E−10 |
| 12 | cg13155430 | MX1 | −9.259439979 | 9.69E−16 | cg20389772 | RSAD2 | 7.56276317 | 7.16E−10 |
| 13 | cg06981309 | PLSCR1 | −8.863520111 | 3.23E−14 | cg10728454 | GPR176 | 7.505328754 | 1.08E−09 |
| 14 | cg16785077 | MX1 | −8.767971093 | 7.15E−14 | cg13409100 | EPSTI1 | 7.42146085 | 0.000000002 |
| 15 | cg26312951 | MX1 | −8.627330695 | 2.34E−13 | cg01434938 | CDKL1 | 7.312842276 | 4.11E−09 |
| 16 | cg12461141 | TRIM22 | −8.46538324 | 8.87E−13 | cg20754708 | | 7.297400934 | 4.51E−09 |
| 17 | cg02650017 | PHOSPHO1 | −8.461802276 | 8.87E−13 | cg15627721 | | 7.093037787 | 1.89E−08 |
| 18 | cg07362849 | BISPR | −8.193961477 | 8.13E−12 | cg15425763 | | 7.056361596 | 2.37E−08 |
| 19 | cg17607231 | SP140 | −8.122764716 | 1.40E−11 | cg25727520 | QPCT | 7.034506881 | 2.72E−08 |
| 20 | cg01028142 | CMPK2 | −8.023069652 | 3.04E−11 | cg10574570 | | 7.020647774 | 2.95E−08 |
| 21 | cg25563198 | FKBP5 | −7.996025138 | 3.64E−11 | cg13937483 | EPSTI1 | 6.993049792 | 3.52E−08 |
| 22 | cg00607627 | LAT | −7.975500336 | 4.13E−11 | cg03318414 | | 6.946502855 | 4.82E−08 |
| 23 | cg03699074 | FAM38A | −7.895994643 | 7.28E−11 | cg12633399 | | 6.900432036 | 6.22E−08 |
| 24 | cg12439472 | EPSTI1 | −7.81856327 | 1.22E−10 | cg07091793 | | 6.825807827 | 1.03E−07 |
| 25 | cg24819835 | CD38 | −7.739887031 | 2.01E−10 | cg01695994 | | 6.785579818 | 1.34E−07 |
| 26 | cg13421019 | PPFIBP2 | −7.693784596 | 2.81E−10 | cg18678177 | ARMC9 | 6.740942189 | 1.80E−07 |
| 27 | cg02138684 | | −7.678871695 | 3.07E−10 | cg18343506 | PACSIN2 | 6.70201733 | 0.000000217 |
| 28 | cg08926253 | IRF7 | −7.478924459 | 1.29E−09 | cg15999041 | | 6.666927773 | 2.72E−07 |
| 29 | cg03879629 | | −7.382408675 | 2.56E−09 | cg19938920 | OSBPL1A | 6.621081084 | 3.60E−07 |
| 30 | cg25114611 | FKBP5 | −7.376980802 | 2.60E−09 | cg04648490 | EXOSC7 | 6.600874993 | 4.07E−07 |
| 31 | cg07815522 | PARP9 | −7.22243086 | 7.69E−09 | cg14237301 | APOB48R | 6.598767923 | 4.07E−07 |
| 32 | cg08084228 | | −7.146112743 | 1.32E−08 | cg26955963 | LINC00299 | 6.588319147 | 0.000000431 |
| 33 | cg16486109 | IRF7 | −7.082434894 | 0.00000002 | cg14528056 | GBAP1 | 6.571432363 | 4.76E−07 |
| 34 | cg10778971 | IFI27 | −6.94203734 | 4.88E−08 | cg08775153 | RUSC2 | 6.545181868 | 5.46E−07 |
| 35 | cg08539067 | GPX1 | −6.915845958 | 5.77E−08 | cg23633330 | CD55 | 6.524284615 | 6.04E−07 |
| 36 | cg21587506 | IFIT3 | −6.903859393 | 6.17E−08 | cg18518074 | EHD1 | 6.514853916 | 6.28E−07 |
| 37 | cg04168577 | PPFIBP2 | −6.726564566 | 0.000000195 | cg06070445 | BCL6 | 6.503682675 | 0.000000668 |
| 38 | cg25998594 | IRF9 | −6.721099477 | 1.99E−07 | cg12685166 | WIPF1 | 6.437972011 | 0.000000969 |
| 39 | cg12331471 | SP100 | −6.717065842 | 2.02E−07 | cg04523589 | CAMP | 6.431503597 | 9.81E−07 |
| 40 | cg24103563 | TRIM34 | −6.705982679 | 2.14E−07 | cg22488164 | PLBD1 | 6.414056356 | 1.07E−06 |
| 41 | cg09747445 | TLE3 | −6.639334924 | 0.000000323 | cg18712971 | | 6.398125563 | 1.17E−06 |
| 42 | cg02176569 | SP140 | −6.549584637 | 5.44E−07 | cg22107082 | | 6.393598124 | 1.19E−06 |
| 43 | cg02233071 | RUNX1 | −6.5469289 | 5.46E−07 | cg21718586 | RSAD2 | 6.39090922 | 1.20E−06 |
| 44 | cg10636246 | AIM2 | −6.540693079 | 5.55E−07 | cg27617715 | FTCDNL1 | 6.370655196 | 1.36E−06 |
| 45 | cg07957619 | GTPBP2 | −6.53261715 | 5.79E−07 | cg01156920 | | 6.369491653 | 1.36E−06 |
| 46 | cg26416615 | ARID5B | −6.520750054 | 6.11E−07 | cg22753522 | | 6.357608795 | 1.44E−06 |
| 47 | cg13452062 | IFI44L | −6.485705541 | 7.44E−07 | cg07124045 | KCNIP2 | 6.357502798 | 1.44E−06 |
| 48 | cg04880620 | OAS2 | −6.46593816 | 8.38E−07 | cg24388175 | PCCA | 6.296139605 | 2.12E−06 |
| 49 | cg02314339 | | −6.464029153 | 8.39E−07 | cg12247536 | OAS2 | 6.282721736 | 2.28E−06 |
| 50 | cg08122652 | PARP9 | −6.43957107 | 0.000000969 | cg19835618 | TPPP3 | 6.278602747 | 2.32E−06 |
| 51 | cg16462073 | SPNS1 | −6.436922347 | 0.000000969 | cg14956920 | VOPP1 | 6.267060809 | 2.46E−06 |
| 52 | cg06703222 | NFAT5 | −6.432375101 | 9.81E−07 | cg12340267 | ARHGAP26 | 6.248597138 | 2.71E−06 |
| 53 | cg18734877 | PTK2B | −6.428472944 | 9.81E−07 | cg27216853 | CYS1 | 6.245458623 | 2.74E−06 |
| 54 | cg19371652 | OAS2 | −6.428313096 | 9.81E−07 | cg00444883 | | 6.229546279 | 3.01E−06 |
| 55 | cg16536330 | LEPROTL1 | −6.268203491 | 2.46E−06 | cg00634542 | SLC11A1 | 6.190788117 | 3.80E−06 |
| 56 | cg09803092 | | −6.263344134 | 0.000002491 | cg06807905 | KCNC4 | 6.189961588 | 3.80E−06 |
| 57 | cg26572165 | CD244 | −6.146822989 | 4.82E−06 | cg17984982 | EPSTI1 | 6.182517045 | 3.95E−06 |
| 58 | cg10447678 | SP110 | −6.072269301 | 7.31E−06 | cg25594515 | IRF2 | 6.178861581 | 4.01E−06 |
| 59 | cg04670072 | | −5.977348793 | 1.21E−05 | cg26508444 | FAM53B | 6.159186087 | 4.50E−06 |
| 60 | cg15052029 | MAZ | −5.950639623 | 1.37E−05 | cg04173586 | DOTIL | 6.137813502 | 5.06E−06 |
| 61 | cg08761339 | IFI27 | −5.925301692 | 0.000015219 | cg26398213 | FGD4 | 6.112640421 | 5.87E−06 |
| 62 | cg26653677 | | −5.925172129 | 0.000015219 | cg06613222 | PRKCE | 6.085401396 | 6.91E−06 |
| 63 | cg06130714 | GAS7 | −5.897516101 | 1.75E−05 | cg03061177 | EPSTI1 | 6.078020366 | 7.17E−06 |
| 64 | cg18101225 | FGFBP2 | −5.885044741 | 1.83E−05 | cg12681784 | BAHCC1 | 6.073126595 | 7.31E−06 |
| 65 | cg24032265 | MIR1204 | −5.776993718 | 3.18E−05 | cg00588694 | | 6.068430848 | 7.40E−06 |
| 66 | cg02889679 | | −5.764002247 | 3.40E−05 | cg03172765 | PSMD1 | 6.066590323 | 7.40E−06 |
| 67 | cg05309505 | IRF7 | −5.742086374 | 3.82E−05 | cg27332633 | | 6.066418255 | 7.40E−06 |
| 68 | cg22432956 | | −5.725962803 | 4.11E−05 | cg19796584 | | 6.054439144 | 7.91E−06 |
| 69 | cg16315329 | CISH | −5.686357792 | 5.04E−05 | cg09553840 | ASAP1 | 6.040240398 | 8.54E−06 |
| 70 | cg16015295 | RPTOR | −5.682647457 | 5.13E−05 | cg24273484 | ROCK1 | 6.03947981 | 8.54E−06 |
| 71 | cg06188083 | IFIT3 | −5.675767096 | 5.31E−05 | cg17867252 | KCNQ5-IT1 | 6.032185203 | 8.87E−06 |
| 72 | cg03887528 | SP140 | −5.659194615 | 5.78E−05 | cg10446995 | | 6.017951771 | 9.61E−06 |
| 73 | cg23540139 | IRF7 | −5.628243735 | 6.81E−05 | cg25004725 | ANXA6 | 6.00328669 | 0.000010441 |
| 74 | cg10959651 | RSAD2 | −5.606429387 | 7.45E−05 | cg15295322 | | 5.994917427 | 1.09E−05 |
| 75 | cg12987761 | USP18 | −5.56691349 | 9.26E−05 | cg20060108 | IL1RL1 | 5.966284537 | 1.28E−05 |
| 76 | cg27634187 | DALRD3 | −5.545977801 | 0.000102823 | cg09426038 | STXBP5-AS1 | 5.962973052 | 0.000012913 |
| 77 | cg25739715 | OSM | −5.541842268 | 0.000104294 | cg17445342 | TKTL2 | 5.962590211 | 0.000012913 |
| 78 | cg05007923 | FNDC3A | −5.518056933 | 0.000117346 | cg19730422 | CYFIP2 | 5.958044837 | 1.32E−05 |
| 79 | cg24772388 | GYPC | −5.512659086 | 0.000119168 | cg05914150 | PIK3R1 | 5.945745423 | 1.40E−05 |
| 80 | cg07046885 | PPP2R5B | −5.508339505 | 0.000121436 | cg20938047 | STXBP6 | 5.939534869 | 1.44E−05 |
| 81 | cg18507060 | OAS3 | −5.504794948 | 0.000123321 | cg23779886 | | 5.939142062 | 1.44E−05 |
| 82 | cg12999836 | GRB10 | −5.490841934 | 0.000132225 | cg13645296 | DAPK2 | 5.936494561 | 1.45E−05 |
| 83 | cg25097419 | SERINC5 | −5.419142048 | 0.000184582 | cg17078207 | NENF | 5.927478597 | 1.52E−05 |
| 84 | cg22734513 | APOL6 | −5.415080473 | 0.000185982 | cg14962159 | SMAD9 | 5.916724555 | 1.59E−05 |
| 85 | cg17986793 | MX1 | −5.411913879 | 0.000187691 | cg10652637 | | 5.905723775 | 0.000016895 |
| 86 | cg07822203 | | −5.404828125 | 0.000191803 | cg27333269 | EFHD2 | 5.898762799 | 1.75E−05 |
| 87 | cg20692268 | | −5.403718507 | 0.000191803 | cg26296574 | B3GNT8 | 5.891874359 | 1.80E−05 |
| 88 | cg01553433 | BST2 | −5.395704244 | 0.000199509 | cg11900509 | ANXA11 | 5.890897316 | 1.80E−05 |
| 89 | cg08847775 | | −5.390788968 | 0.000203364 | cg14209284 | | 5.888892205 | 1.81E−05 |
| 90 | cg26800289 | | −5.379984346 | 0.000214199 | cg09130161 | FAM129C | 5.884948504 | 1.83E−05 |
| 91 | cg18034719 | GRK6 | −5.368615015 | 0.0002254 | cg26426564 | INPP5A | 5.861727255 | 2.07E−05 |
| 92 | cg16400320 | | −5.353197743 | 0.000243273 | cg15480653 | FNBP1 | 5.861594711 | 2.07E−05 |
| 93 | cg12088946 | TRIM6-TRIM34 | −5.348583346 | 0.000245899 | cg22508829 | TOMIL2 | 5.861562059 | 2.07E−05 |
| 94 | cg12159504 | STAT1 | −5.339799227 | 0.00025413 | cg04326337 | RPRD1B | 5.855345112 | 2.13E−05 |
| 95 | cg05859076 | RAB43 | −5.330595448 | 0.000266325 | cg24781413 | | 5.849658565 | 2.19E−05 |
| 96 | cg04268125 | ADAR | −5.311358735 | 0.000292656 | cg25963583 | MAX | 5.844585817 | 2.25E−05 |
| 97 | cg11170544 | TTC22 | −5.282991172 | 0.000331733 | cg20928986 | SP110 | 5.841554318 | 2.27E−05 |
| 98 | cg16399664 | OAS2 | −5.252720455 | 0.000381336 | cg09256413 | NEGR1 | 5.824200034 | 2.51E−05 |
| 99 | cg14353998 | OASL | −5.232184768 | 0.000411444 | cg02881166 | | 5.814076437 | 2.65E−05 |
| 100 | cg02276017 | PSMA1 | −5.222155052 | 0.000428421 | cg01745810 | DENNDIA | 5.811714457 | 2.67E−05 |

EarlyPost-Control

| Hypo Methylated | | | | | Hyper Methylated | | | |
|---|---|---|---|---|---|---|---|---|
| Rank | CpG | Gene | T | Adj. P. Val | CpG | Gene | T | Adj. P Val |
| 1 | cg26312951 | MX1 | −1.35E+01 | 8.28E−36 | cg26505274 | OASL | 9.74E+00 | 6.38E−18 |
| 2 | cg13452062 | IFI44L | −1.33E+01 | 1.02E−34 | cg04648490 | EXOSC7 | 8.62E+00 | 1.55E−13 |
| 3 | cg06981309 | PLSCR1 | −1.21E+01 | 1.65E−28 | cg07897699 | ITPR1 | 8.62E+00 | 1.55E−13 |
| 4 | cg12439472 | EPSTI1 | −1.16E+01 | 4.97E−26 | cg13849515 | MIR3614 | 8.53E+00 | 3.14E−13 |
| 5 | cg01028142 | CMPK2 | −1.16E+01 | 6.83E−26 | cg27333269 | EFHD2 | 8.05E+00 | 1.36E−11 |
| 6 | cg22930808 | PARP9 | −1.14E+01 | 7.37E−25 | cg04392554 | TTC38 | 7.99E+00 | 2.22E−11 |
| 7 | cg21549285 | MX1 | −1.13E+01 | 2.20E−24 | cg01616956 | NMUR1 | 7.95E+00 | 2.90E−11 |
| 8 | cg00959259 | PARP9 | −1.12E+01 | 3.71E−24 | cg24137511 | MAST3 | 7.88E+00 | 5.32E−11 |
| 9 | cg08122652 | PARP9 | −1.12E+01 | 3.90E−24 | cg06706159 | MAST3 | 7.84E+00 | 6.86E−11 |
| 10 | cg13155430 | MX1 | −1.12E+01 | 4.62E−24 | cg04213565 | | 7.82E+00 | 7.51E−11 |
| 11 | cg03607951 | IFI44L | −1.11E+01 | 5.57E−24 | cg14410991 | | 7.79E+00 | 9.82E−11 |
| 12 | cg05523603 | | −1.11E+01 | 1.16E−23 | cg21659576 | | 7.69E+00 | 2.03E−10 |
| 13 | cg22862003 | MX1 | −1.08E+01 | 1.12E−22 | cg16404170 | FAM53B | 7.62E+00 | 3.30E−10 |
| 14 | cg13304609 | IFI44L | −1.07E+01 | 3.51E−22 | cg22176018 | | 7.57E+00 | 4.70E−10 |
| 15 | cg10549986 | RSAD2 | −1.06E+01 | 8.27E−22 | cg07023764 | CCDC26 | 7.57E+00 | 4.70E−10 |
| 16 | cg07815522 | PARP9 | −1.06E+01 | 8.27E−22 | cg00634542 | SLC11A1 | 7.46E+00 | 1.07E−09 |
| 17 | cg07362849 | BISPR | −1.05E+01 | 3.40E−21 | cg00444883 | | 7.45E+00 | 1.09E−09 |
| 18 | cg12987761 | USP18 | −1.03E+01 | 2.45E−20 | cg22069247 | NMUR1 | 7.42E+00 | 1.34E−09 |
| 19 | cg24678928 | DDX60 | −1.01E+01 | 2.26E−19 | cg13707793 | CXXC5 | 7.38E+00 | 1.82E−09 |
| 20 | cg12013713 | PARP12 | −9.96E+00 | 8.15E−19 | cg18218829 | TRAF3IP1 | 7.36E+00 | 2.12E−09 |
| 21 | cg03879629 | | −9.90E+00 | 1.40E−18 | cg10446995 | | 7.35E+00 | 2.20E−09 |
| 22 | cg08926253 | IRF7 | −9.40E+00 | 1.76E−16 | cg13917614 | CNP | 7.307219301 | 0.000000003 |
| 23 | cg03753191 | EPSTI1 | −9.30E+00 | 4.12E−16 | cg14284762 | PIK3R1 | 7.305502989 | 0.000000003 |
| 24 | cg16400320 | | −9.03E+00 | 4.76E−15 | cg23369683 | | 7.29E+00 | 3.15E−09 |
| 25 | cg16644494 | ODF3B | −8.89E+00 | 1.70E−14 | cg24620635 | GNLY | 7.28E+00 | 3.36E−09 |
| 26 | cg05696877 | IFI44L | −8.72E+00 | 7.59E−14 | cg14495063 | PFKFB4 | 7.28E+00 | 3.36E−09 |
| 27 | cg11829870 | KLHDC7B | −8.69E+00 | 8.78E−14 | cg24644262 | LOC100507391 | 7.25E+00 | 4.20E−09 |
| 28 | cg19371652 | OAS2 | −8.65E+00 | 1.23E−13 | cg23352030 | PRIC285 | 7.22E+00 | 5.26E−09 |
| 29 | cg16785077 | MX1 | −8.60E+00 | 1.79E−13 | cg24603130 | ZCCHC2 | 7.15E+00 | 8.36E−09 |
| 30 | cg12331471 | SP100 | −8.50E+00 | 4.05E−13 | cg22202022 | | 7.10E+00 | 1.14E−08 |
| 31 | cg11251971 | CYSTM1 | −8.42E+00 | 7.85E−13 | cg08781187 | | 7.07E+00 | 1.47E−08 |
| 32 | cg05552874 | IFIT1 | −8.38E+00 | 1.01E−12 | cg21515766 | STK10 | 7.05E+00 | 1.57E−08 |
| 33 | cg08084228 | | −8.38E+00 | 1.01E−12 | cg13617280 | SLC15A4 | 7.05E+00 | 1.65E−08 |
| 34 | cg12906975 | | −8.33E+00 | 1.47E−12 | cg23598089 | ATP2B4 | 7.04E+00 | 1.67E−08 |
| 35 | cg06188083 | IFIT3 | −8.31E+00 | 1.67E−12 | cg08471335 | TTC38 | 7.04E+00 | 1.69E−08 |
| 36 | cg04268125 | ADAR | −8.27E+00 | 2.29E−12 | cg13693517 | TASP1 | 7.04E+00 | 1.69E−08 |
| 37 | cg01553433 | BST2 | −8.06E+00 | 1.36E−11 | cg08433366 | RFFL | 7.03193714 | 0.000000017 |
| 38 | cg02314339 | | −7.86E+00 | 5.78E−11 | cg26508444 | FAM53B | 7.031500052 | 0.000000017 |
| 39 | cg12461141 | TRIM22 | −7.75E+00 | 1.27E−10 | cg04873169 | GRAMD4 | 6.96E+00 | 2.79E−08 |
| 40 | cg08888522 | IFIH1 | −7.71E+00 | 1.80E−10 | cg01434938 | CDKL1 | 6.95E+00 | 2.88E−08 |
| 41 | cg02247863 | | −7.54E+00 | 5.81E−10 | cg05251703 | GATAD2A | 6.94E+00 | 3.16E−08 |
| 42 | cg25998594 | IRF9 | −7.46E+00 | 1.07E−09 | cg02003183 | CDC42BPB | 6.93E+00 | 3.31E−08 |
| 43 | cg10552523 | IFITM1 | −7.30E+00 | 3.15E−09 | cg11643361 | | 6.91E+00 | 3.78E−08 |
| 44 | cg01190666 | PRIC285 | −7.21E+00 | 5.43E−09 | cg04602990 | PFKFB4 | 6.86E+00 | 5.06E−08 |
| 45 | cg14943355 | PARP11 | −7.11E+00 | 1.14E−08 | cg06826636 | | 6.85E+00 | 5.19E−08 |
| 46 | cg00458211 | IFI44L | −7.095343379 | 0.000000012 | cg18011382 | | 6.839771188 | 0.000000055 |
| 47 | cg10778971 | IFI27 | −7.03224009 | 0.000000017 | cg02183564 | CCDC146 | 6.83E+00 | 5.72E−08 |
| 48 | cg12424383 | | −7.03E+00 | 1.76E−08 | cg03180724 | | 6.83E+00 | 5.84E−08 |
| 49 | cg09747445 | TLE3 | −7.01E+00 | 1.92E−08 | cg26277237 | KANK1 | 6.82E+00 | 6.06E−08 |
| 50 | cg02233071 | RUNX1 | −6.92E+00 | 3.45E−08 | cg06296597 | LARP4B | 6.77E+00 | 8.09E−08 |
| 51 | cg04880620 | OAS2 | −6.90E+00 | 3.89E−08 | cg06588802 | | 6.74897446 | 0.000000094 |
| 52 | cg01079652 | IFI44 | −6.89E+00 | 4.08E−08 | cg15690475 | MAPT | 6.72E+00 | 1.14E−07 |
| 53 | cg26204448 | MADIL1 | −6.89E+00 | 4.22E−08 | cg25965344 | TSPAN2 | 6.71E+00 | 1.20E−07 |
| 54 | cg14595557 | CMPK2 | −6.87E+00 | 4.63E−08 | cg00910503 | HEXDC | 6.709660615 | 0.00000012 |
| 55 | cg25563198 | FKBP5 | −6.86E+00 | 4.92E−08 | cg14259466 | ADAM8 | 6.695619388 | 0.000000131 |
| 56 | cg04670072 | | −6.84E+00 | 5.38E−08 | cg11296110 | NADK | 6.69E+00 | 1.32E−07 |
| 57 | cg17986793 | MX1 | −6.83E+00 | 5.84E−08 | cg07151386 | GMDS | 6.69E+00 | 1.36E−07 |
| 58 | cg03546163 | FKBP5 | −6.82E+00 | 5.98E−08 | cg19149463 | VGLL4 | 6.68E+00 | 1.40E−07 |
| 59 | cg09858955 | VRK2 | −6.803219815 | 0.000000067 | cg04987734 | CDC42BPB | 6.68E+00 | 1.40E−07 |
| 60 | cg14870271 | LGALS3BP | −6.79E+00 | 7.51E−08 | cg18476689 | | 6.647751946 | 0.000000174 |
| 61 | cg22485558 | MX1 | −6.75E+00 | 9.11E−08 | cg15779457 | | 6.64E+00 | 1.86E−07 |
| 62 | cg11791770 | PHRF1 | −6.64E+00 | 1.86E−07 | cg00504500 | FAM49B | 6.63E+00 | 1.95E−07 |
| 63 | cg18543074 | | −6.61E+00 | 2.18E−07 | cg12011479 | CLDND2 | 6.63E+00 | 1.95E−07 |
| 64 | cg07596065 | | −6.57E+00 | 2.65E−07 | cg19796584 | | 6.62E+00 | 2.05E−07 |
| 65 | cg10959651 | RSAD2 | −6.53E+00 | 3.30E−07 | cg26648465 | KIAA0182 | 6.61E+00 | 2.18E−07 |
| 66 | cg24103563 | TRIM34 | −6.50E+00 | 4.04E−07 | cg24408769 | JARID2 | 6.60E+00 | 2.18E−07 |
| 67 | cg00570504 | | −6.44E+00 | 5.66E−07 | cg12756150 | | 6.56E+00 | 2.90E−07 |
| 68 | cg22016995 | IRF7 | −6.40E+00 | 7.06E−07 | cg10165241 | DPP9 | 6.56E+00 | 2.90E−07 |
| 69 | cg22282590 | BST2 | −6.33E+00 | 1.07E−06 | cg26122975 | | 6.56E+00 | 2.96E−07 |
| 70 | cg00381193 | KCNIP1 | −6.319699669 | 0.000001117 | cg18247172 | | 6.55E+00 | 3.10E−07 |
| 71 | cg12999836 | GRB10 | −6.319664425 | 0.000001117 | cg27363280 | | 6.54E+00 | 3.16E−07 |
| 72 | cg12557158 | | −6.25E+00 | 1.59E−06 | cg22917487 | CX3CR1 | 6.54E+00 | 3.16E−07 |
| 73 | cg08585593 | TYMP | −6.147105948 | 0.000002777 | cg05117620 | CMKLR1 | 6.50E+00 | 4.05E−07 |
| 74 | cg01586797 | CNR2 | −6.12E+00 | 3.22E−06 | cg12332239 | AMZ2 | 6.49E+00 | 4.23E−07 |
| 75 | cg25114611 | FKBP5 | −6.11E+00 | 3.30E−06 | cg03136023 | IQCE | 6.49E+00 | 4.34E−07 |
| 76 | cg14201707 | | −6.07E+00 | 3.97E−06 | cg17022038 | MAST3 | 6.49E+00 | 4.35E−07 |
| 77 | cg05085499 | BTBD19 | −6.07E+00 | 4.01E−06 | cg24598860 | MECOM | 6.49E+00 | 4.35E−07 |
| 78 | cg17202840 | | −6.068907257 | 0.000004085 | cg13775629 | PRF1 | 6.49E+00 | 4.35E−07 |
| 79 | cg01680062 | RUNX1 | −6.05E+00 | 4.50E−06 | cg14721093 | EFHD2 | 6.48E+00 | 4.43E−07 |
| 80 | cg10274453 | | −5.99E+00 | 6.05E−06 | cg17408993 | GNS | 6.48E+00 | 4.49E−07 |
| 81 | cg18881723 | SLAMF1 | −5.96E+00 | 6.90E−06 | cg14058851 | JARID2 | 6.47E+00 | 4.61E−07 |
| 82 | cg26971042 | TLE3 | −5.94E+00 | 7.40E−06 | cg08087047 | CD300A | 6.47E+00 | 4.68E−07 |
| 83 | cg02491794 | TTC7A | −5.94E+00 | 7.54E−06 | cg03059896 | WDTC1 | 6.46E+00 | 5.01E−07 |
| 84 | cg14428587 | PARP12 | −5.94E+00 | 7.61E−06 | cg01000422 | PHKB | 6.44E+00 | 5.57E−07 |
| 85 | cg26882438 | PARP14 | −5.91E+00 | 8.60E−06 | cg07655126 | LYST | 6.44E+00 | 5.60E−07 |
| 86 | cg08244262 | MX1 | −5.90E+00 | 8.77E−06 | cg05065849 | GNLY | 6.44E+00 | 5.66E−07 |
| 87 | cg25867318 | STAT3 | −5.90E+00 | 8.77E−06 | cg15942206 | DENND3 | 6.43E+00 | 5.74E−07 |
| 88 | cg07833467 | KLHDC7B | −5.90E+00 | 8.98E−06 | cg05669550 | CHSY1 | 6.40E+00 | 7.26E−07 |
| 89 | cg23195687 | | −5.89E+00 | 9.28E−06 | cg06019050 | LIN54 | 6.38E+00 | 7.79E−07 |
| 90 | cg11115622 | PLEKHG3 | −5.88E+00 | 9.62E−06 | cg23484980 | | 6.38E+00 | 8.10E−07 |
| 91 | cg06708931 | | −5.86E+00 | 1.06E−05 | cg25004725 | ANXA6 | 6.37E+00 | 8.30E−07 |
| 92 | cg21406720 | | −5.86E+00 | 1.09E−05 | cg03048488 | | 6.351279926 | 0.000000944 |
| 93 | cg02560388 | | −5.82E+00 | 1.34E−05 | cg23344769 | | 6.34E+00 | 1.04E−06 |
| 94 | cg13755924 | MX1 | −5.79E+00 | 1.51E−05 | cg12439163 | | 6.32E+00 | 1.10E−06 |
| 95 | cg03743205 | ZFPM1 | −5.774975169 | 0.000016107 | cg00656410 | SDF4 | 6.320031751 | 0.000001117 |
| 96 | cg05883128 | DDX60 | −5.77E+00 | 1.63E−05 | cg11133963 | ABI3 | 6.313294902 | 0.000001157 |
| 97 | cg16411857 | NLRC5 | −5.77E+00 | 1.66E−05 | cg07675998 | | 6.30E+00 | 1.25E−06 |
| 98 | cg03258567 | | −5.74E+00 | 1.88E−05 | cg14496375 | CLDND2 | 6.30E+00 | 1.28E−06 |
| 99 | cg14333162 | RSAD2 | −5.74E+00 | 1.92E−05 | cg03325407 | BCL2L15 | 6.29E+00 | 1.29E−06 |
| 100 | cg08724920 | BCL11B | −5.70E+00 | 2.35E−05 | cg17008273 | LOC101928682 | 6.29E+00 | 1.31E−06 |

Late-Post Control

| Hypo methylated | | | | | Hyper methylated | | | |
|---|---|---|---|---|---|---|---|---|
| Rank | CpG | Gene | T | Adj. P. Val | CpG | Gene | T | Adj. P Val |
| 1 | cg03607951 | IFI44L | −9.54287288 | 9.83E−16 | cg04213565 | | 7.040833395 | 3.38E−07 |
| 2 | cg05696877 | IFI44L | −7.774558844 | 2.68E−09 | cg01695994 | | 6.746832801 | 1.78E−06 |
| 3 | cg13452062 | IFI44L | −7.277305287 | 8.03E−08 | cg26505274 | OASL | 6.400163042 | 1.57E−05 |
| 4 | cg03546163 | FKBP5 | −6.778973106 | 1.71E−06 | cg07675998 | | 6.257035136 | 0.000034693 |
| 5 | cg06981309 | PLSCR1 | −6.210596229 | 3.73E−05 | cg15730234 | | 6.214922604 | 3.73E−05 |
| 6 | cg14201707 | | −5.955133301 | 0.000132106 | cg17944885 | | 6.125888659 | 5.80E−05 |
| 7 | cg27345524 | SMAD3 | −5.725181526 | 0.000334817 | cg24620635 | GNLY | 6.084839396 | 6.87E−05 |
| 8 | cg26312951 | MX1 | −5.712803328 | 0.000341779 | cg27333269 | EFHD2 | 5.954127796 | 0.000132106 |
| 9 | cg24678928 | DDX60 | −5.585950775 | 0.000608921 | cg00504500 | FAM49B | 5.914788221 | 0.000156706 |
| 10 | cg14084689 | SORCS2 | −5.563766069 | 0.000666976 | cg13917614 | CNP | 5.900312669 | 0.0001604 |
| 11 | cg22298224 | SART1 | −5.291117129 | 0.001862735 | cg21659576 | | 5.857082764 | 0.000194986 |
| 12 | cg12984893 | INPP5A | −5.223558334 | 0.002299149 | cg05251703 | GATAD2A | 5.848449815 | 0.000194986 |
| 13 | cg21254939 | SORCS2 | −5.207188423 | 0.002405364 | cg04392554 | TTC38 | 5.79498924 | 0.000254373 |
| 14 | cg25563198 | FKBP5 | −5.191323181 | 0.002503398 | cg07023764 | CCDC26 | 5.752105921 | 0.000311729 |
| 15 | cg18734877 | PTK2B | −5.137506854 | 0.003176365 | cg12550496 | | 5.723855726 | 0.000334817 |
| 16 | cg11989845 | PCNT | −5.113137045 | 0.003434612 | cg19893929 | | 5.689204726 | 0.000366334 |
| 17 | cg12439472 | EPSTI1 | −5.011210481 | 0.004782534 | cg23598089 | ATP2B4 | 5.686760425 | 0.000366334 |
| 18 | cg23394259 | DGKA | −4.994887145 | 0.00488863 | cg01616956 | NMUR1 | 5.588049074 | 0.000608921 |
| 19 | cg17725019 | PIK3IP1 | −4.985991794 | 0.004961691 | cg18123613 | WNK2 | 5.548200394 | 0.000704008 |
| 20 | cg12999836 | GRB10 | −4.959687833 | 0.005437436 | cg24137511 | MAST3 | 5.525020898 | 0.000757912 |
| 21 | cg09747445 | TLE3 | −4.933585288 | 0.006075314 | cg24825894 | PIK3AP1 | 5.523583812 | 0.000757912 |
| 22 | cg25103161 | MGAT4B | −4.915591799 | 0.00645483 | cg19796584 | | 5.515684011 | 0.000767987 |
| 23 | cg21770157 | CYB5D1 | −4.911515993 | 0.006523237 | cg21515766 | STK10 | 5.440118155 | 0.001141322 |
| 24 | cg19477793 | | −4.897675193 | 0.00685979 | cg13893448 | PAM | 5.432821675 | 0.001154036 |
| 25 | cg10671195 | TAPT1 | −4.870901246 | 0.007629226 | cg12332239 | AMZ2 | 5.393158485 | 0.001361053 |
| 26 | cg10771443 | RSAD2 | −4.839277213 | 0.008304306 | cg27495654 | SPIDR | 5.390199978 | 0.001361053 |
| 27 | cg02708898 | ATP6V1H | −4.826422915 | 0.008476577 | cg07897699 | ITPR1 | 5.388134692 | 0.001361053 |
| 28 | cg12331471 | SP100 | −4.814851286 | 0.008756394 | cg17705647 | FOXN3 | 5.383322846 | 0.001361172 |
| 29 | cg13344434 | FKBP5 | −4.793244629 | 0.009593534 | cg03299208 | FAH | 5.373479081 | 0.001400789 |
| 30 | cg01297684 | | −4.788125693 | 0.009681431 | cg08087047 | CD300A | 5.349847163 | 0.0015567 |
| 31 | cg15975802 | PTPN6 | −4.767772883 | 0.010135596 | cg03048488 | | 5.333962746 | 0.001657853 |
| 32 | cg09858955 | VRK2 | −4.725018765 | 0.011771191 | cg06706159 | MAST3 | 5.325938504 | 0.001691489 |
| 33 | cg12992827 | | −4.720174411 | 0.011906028 | cg11335172 | IL18RAP | 5.315871692 | 0.001733296 |
| 34 | cg05085499 | BTBD19 | −4.714910347 | 0.012045925 | cg20540694 | CACNA2D2 | 5.313031874 | 0.001733296 |
| 35 | cg01140711 | | −4.71149388 | 0.012164017 | cg05653887 | CELSR1 | 5.293156174 | 0.001862735 |
| 36 | cg23643435 | GALM | −4.707751654 | 0.012218519 | cg04648490 | EXOSC7 | 5.28580053 | 0.001862735 |
| 37 | cg00151661 | NOTCH1 | −4.694475967 | 0.012689351 | cg22917487 | CX3CR1 | 5.283988581 | 0.001862735 |
| 38 | cg00959259 | PARP9 | −4.685402851 | 0.013002874 | cg02183564 | CCDC146 | 5.278133426 | 0.001883984 |
| 39 | cg12054453 | TMEM49 | −4.674360427 | 0.013179218 | cg06068163 | EIF3B | 5.248550362 | 0.002145743 |
| 40 | cg14011327 | | −4.674085462 | 0.013179218 | cg04602990 | PFKFB4 | 5.246869089 | 0.002145743 |
| 41 | cg05859076 | RAB43 | −4.666501127 | 0.013377511 | cg23369683 | | 5.236512041 | 0.002197738 |
| 42 | cg06188083 | IFIT3 | −4.66081342 | 0.013668223 | cg07729082 | ZC3H18 | 5.235354575 | 0.002197738 |
| 43 | cg05552874 | IFIT1 | −4.657230278 | 0.013823388 | cg14495063 | PFKFB4 | 5.212209147 | 0.002399949 |
| 44 | cg18881723 | SLAMF1 | −4.616629835 | 0.015595435 | cg04315689 | DGUOK-AS1 | 5.203937533 | 0.002405364 |
| 45 | cg04729913 | FLJ45983 | −4.616514955 | 0.015595435 | cg25885684 | | 5.20193194 | 0.002405364 |
| 46 | cg08724920 | BCL11B | −4.553554981 | 0.018412527 | cg26277237 | KANK1 | 5.167337842 | 0.002799405 |
| 47 | cg16651877 | BTBD19 | −4.543613242 | 0.018774645 | cg19795996 | ZEB2 | 5.164210027 | 0.002799951 |
| 48 | cg13421019 | PPFIBP2 | −4.543025937 | 0.018774645 | cg17211761 | | 5.130900349 | 0.003230183 |
| 49 | cg05496248 | RGS10 | −4.490010349 | 0.021506336 | cg23352030 | PRIC285 | 5.128372647 | 0.003230183 |
| 50 | cg24599759 | | −4.489570692 | 0.021506336 | cg16404170 | FAM53B | 5.11099605 | 0.003434612 |
| 51 | cg00532633 | CFDP1 | −4.445016708 | 0.024189288 | cg14314770 | | 5.107110069 | 0.003453647 |
| 52 | cg17333883 | LINC01227 | −4.429647602 | 0.024897242 | cg11133963 | ABI3 | 5.102946118 | 0.003478619 |
| 53 | cg02100984 | | −4.429594457 | 0.024897242 | cg12535090 | NAV2 | 5.093150884 | 0.003571821 |
| 54 | cg05449373 | AMICA1 | −4.429585184 | 0.024897242 | cg04002181 | ARSA | 5.09245055 | 0.003571821 |
| 55 | cg13163919 | TLE3 | −4.417679428 | 0.025670196 | cg24297901 | VPS53 | 5.077592454 | 0.003776247 |
| 56 | cg16531578 | TRERF1 | −4.417610429 | 0.025670196 | cg07208891 | KLHDC4 | 5.076539084 | 0.003776247 |
| 57 | cg11727482 | PELI1 | −4.416826377 | 0.025670196 | cg13707793 | CXXC5 | 5.073742507 | 0.003779702 |
| 58 | cg13271663 | TLE3 | −4.409712089 | 0.026104649 | cg17972058 | SLAMF8 | 5.061857362 | 0.003968791 |
| 59 | cg14864167 | PDE7A | −4.402628232 | 0.026731418 | cg08781187 | | 5.057067084 | 0.004015488 |
| 60 | cg06398873 | | −4.400600764 | 0.026771795 | cg13617280 | SLC15A4 | 5.043250107 | 0.004246824 |
| 61 | cg06520846 | PPCS | −4.396909502 | 0.026872245 | cg07039378 | | 5.041337629 | 0.004246824 |
| 62 | cg07603058 | BTBD19 | −4.385752788 | 0.027904637 | cg01000422 | PHKB | 5.038414424 | 0.004256903 |
| 63 | cg14775730 | SPEF2 | −4.380792734 | 0.028450433 | cg01112784 | RHOBTB1 | 5.021029733 | 0.004601897 |
| 64 | cg21796654 | PLA2G6 | −4.377466728 | 0.028513182 | cg07675950 | AKNA | 5.004511852 | 0.004878993 |
| 65 | cg26562462 | TBC1D14 | −4.376216467 | 0.02856819 | cg22146755 | | 5.000437234 | 0.004878993 |
| 66 | cg02512559 | CFDP1 | −4.368385174 | 0.029124807 | cg26955383 | CALHM1 | 5.000274072 | 0.004878993 |
| 67 | cg13575601 | ARHGAP25 | −4.346647868 | 0.030461459 | cg08854185 | DBNDD1 | 4.993936495 | 0.00488863 |
| 68 | cg23781022 | ELK3 | −4.340620207 | 0.030923899 | cg22282161 | DNAH11 | 4.993043139 | 0.00488863 |
| 69 | cg22862003 | MX1 | −4.311749155 | 0.033554455 | cg14284762 | PIK3R1 | 4.985735908 | 0.004961691 |
| 70 | cg24772388 | GYPC | −4.309011521 | 0.03370291 | cg14410991 | | 4.979976958 | 0.005054204 |
| 71 | cg23930334 | SLC22A20 | −4.298669163 | 0.034010232 | cg27509293 | | 4.971670669 | 0.005217057 |
| 72 | cg14235698 | SRP14-AS1 | −4.297662458 | 0.034010232 | cg24603130 | ZCCHC2 | 4.959376651 | 0.005437436 |
| 73 | cg04356230 | HIVEP2 | −4.288927197 | 0.035277324 | cg13849515 | MIR3614 | 4.955320913 | 0.005492395 |
| 74 | cg13719443 | LOC285847 | −4.286491361 | 0.035332437 | cg11407606 | FAM92B | 4.92450698 | 0.006253116 |
| 75 | cg02111474 | PPFIA1 | −4.284906962 | 0.035332437 | cg24408769 | JARID2 | 4.923834629 | 0.006253116 |
| 76 | cg04253214 | | −4.274857794 | 0.036382024 | cg06727233 | | 4.908568463 | 0.00655514 |
| 77 | cg19996254 | ABLIM1 | −4.269729035 | 0.036916821 | cg15472170 | PLOD1 | 4.895014033 | 0.006884427 |
| 78 | cg10549986 | RSAD2 | −4.268255234 | 0.037020903 | cg02474460 | JAZF1-AS1 | 4.884009828 | 0.007208837 |
| 79 | cg08552486 | | −4.260824002 | 0.037518619 | cg03640796 | ITGB5 | 4.867103309 | 0.007674085 |
| 80 | cg01818341 | SMAD3 | −4.253582949 | 0.037984762 | cg19693031 | TXNIP | 4.865941284 | 0.007674085 |
| 81 | cg24958791 | | −4.229390053 | 0.040919915 | cg12011479 | CLDND2 | 4.86346776 | 0.007697348 |
| 82 | cg08244262 | MX1 | −4.203831568 | 0.044160314 | cg15690475 | MAPT | 4.857663125 | 0.007852324 |
| 83 | cg04708975 | | −4.203491663 | 0.044160314 | cg07184627 | MBP | 4.855671676 | 0.007858219 |
| 84 | cg19790640 | BAALC | −4.200890156 | 0.044250704 | cg22365240 | | 4.851314461 | 0.007959172 |
| 85 | cg15136568 | SHC1 | −4.19643299 | 0.044709126 | cg14496375 | CLDND2 | 4.848999187 | 0.007979417 |
| 86 | cg26867393 | GTF2E2 | −4.179040564 | 0.046875814 | cg15876825 | VGLL4 | 4.832200962 | 0.008390635 |
| 87 | cg03882182 | LYPLAL1-AS1 | −4.176794582 | 0.047100982 | cg24606374 | NCOA7 | 4.83078645 | 0.008390635 |
| 88 | cg24254387 | BTBD19 | −4.172733288 | 0.047520649 | cg22069247 | NMUR1 | 4.830343024 | 0.008390635 |
| 89 | cg04621042 | BRD1 | −4.165366995 | 0.047941188 | cg00141498 | CCDC26 | 4.830177172 | 0.008390635 |
| 90 | cg01028142 | CMPK2 | −4.163215586 | 0.047941188 | cg08297631 | | 4.816942659 | 0.008756394 |
| 91 | cg24996958 | ENPP6 | −4.159826882 | 0.048345416 | cg07885971 | ADPGK-AS1 | 4.816445959 | 0.008756394 |
| 92 | cg25114611 | FKBP5 | −4.159823897 | 0.048345416 | cg02555727 | SLC15A4 | 4.807489565 | 0.009009272 |
| 93 | cg27122888 | NRXN2 | −4.152999625 | 0.049361076 | cg08458637 | TSNAX-DISC1 | 4.790545064 | 0.00964382 |
| 94 | | | | | cg11866539 | MGAT5 | 4.780467305 | 0.009976544 |
| 95 | | | | | cg18310515 | GNLY | 4.77295189 | 0.010135596 |
| 96 | | | | | cg15789789 | TMEM184B | 4.770976872 | 0.010135596 |
| 97 | | | | | cg00910503 | HEXDC | 4.769313869 | 0.010135596 |
| 98 | | | | | cg13383335 | TRIM44 | 4.7692794 | 0.010135596 |
| 99 | | | | | cg00656410 | SDF4 | 4.768842733 | 0.010135596 |
| 100 | | | | | cg01329939 | ANO2 | 4.764060667 | 0.010245153 |

TABLE 2.4
(Top 100 DMS detected over time relative to pre-infection Control.
Data were corrected for cell type proportions; FDR < 0.05)
First-Control

| Hypo methylated | | | | | Hyper methylated | | | |
|---|---|---|---|---|---|---|---|---|
| Rank | CpG | Gene | T | Adj. P. Val | CpG | Gene | T | Adj. P. Val. |
| 1 | cg02650017 | PHOSPHO1 | −8.787943573 | 2.42E−11 | cg16550184 | CLEC4A | 5.692146048 | 0.000953241 |
| 2 | cg10161996 | | −8.634830768 | 3.83E−11 | cg04523589 | CAMP | 5.65216609 | 0.001119076 |
| 3 | cg17114584 | IRF7 | −8.546092543 | 4.94E−11 | cg21805788 | FAM102B | 5.329389901 | 0.004807398 |
| 4 | cg07878065 | | −8.385539382 | 1.21E−10 | cg13821176 | TRIB1 | 5.292729741 | 0.005177794 |
| 5 | cg24002003 | | −8.351960585 | 1.24E−10 | cg10570276 | | 5.191622742 | 0.008009741 |
| 6 | cg02138684 | | −7.637295253 | 1.64E−08 | cg22821289 | | 5.167552769 | 0.008489195 |
| 7 | cg13504881 | | −7.036051644 | 7.67E−07 | cg18343506 | PACSIN2 | 5.048489134 | 0.013188679 |
| 8 | cg00607627 | LAT | −6.698918598 | 5.67E−06 | cg13545732 | EFCAB2 | 5.022306913 | 0.014591407 |
| 9 | cg21587506 | IFIT3 | −6.632710118 | 7.60E−06 | cg27216853 | CYS1 | 5.001449341 | 0.015316476 |
| 10 | cg13575798 | SND1 | −6.569535698 | 1.01E−05 | cg18985251 | | 4.963673338 | 0.017954003 |
| 11 | cg26416615 | ARID5B | −6.460529403 | 1.78E−05 | cg11900509 | ANXA11 | 4.957248998 | 0.01806214 |
| 12 | cg25114611 | FKBP5 | 6.301367879 | 4.22E−05 | cg03318414 | | 4.917616957 | 0.021362031 |
| 13 | cg08539067 | GPX1 | −6.273271178 | 4.60E−05 | cg16867702 | TRIO | 4.899073214 | 0.022808749 |
| 14 | cg03753191 | EPSTI1 | −6.218887009 | 5.87E−05 | cg16431791 | ADGRE3 | 4.863191472 | 0.026476126 |
| 15 | cg13421019 | PPFIBP2 | −6.009216904 | 0.000184207 | cg19796584 | | 4.812286507 | 0.031205794 |
| 16 | cg21806732 | SEPT5 | −5.80410789 | 0.000547121 | cg06070445 | BCL6 | 4.810140063 | 0.031205794 |
| 17 | cg09674502 | GFI1 | −5.624472373 | 0.001231707 | cg00836271 | SHCBP1L | 4.739748077 | 0.040905944 |
| 18 | cg03699074 | FAM38A | −5.499550642 | 0.002284452 | | | | |
| 19 | cg20692268 | | −5.462809138 | 0.002642632 | | | | |
| 20 | cg07957619 | GTPBP2 | −5.360546065 | 0.004309546 | | | | |
| 21 | cg21549285 | MX1 | 5.322656386 | 0.004807398 | | | | |
| 22 | cg04864378 | | −5.30798091 | 0.004978219 | | | | |
| 23 | cg09421562 | MPO | −5.223140372 | 0.007117796 | | | | |
| 24 | cg25563198 | FKBP5 | −5.185777529 | 0.008009741 | | | | |
| 25 | cg22432956 | | −5.134821572 | 0.009685518 | | | | |
| 26 | cg22323734 | | −5.123593164 | 0.009926024 | | | | |
| 27 | cg10636246 | AIM2 | −5.080372222 | 0.011942041 | | | | |
| 28 | cg16462073 | SPNS1 | −5.050667816 | 0.013188679 | | | | |
| 29 | cg05455036 | | −5.012342217 | 0.014910676 | | | | |
| 30 | cg09967176 | | −4.833692022 | 0.029796495 | | | | |
| 31 | cg06981309 | PLSCR1 | 4.813440639 | 0.031205794 | | | | |
| 32 | cg09342060 | HOXA9 | −4.768655324 | 0.03718945 | | | | |
| 33 | cg05316065 | GSDMC | −4.750941268 | 0.039601654 | | | | |
| 34 | cg09678315 | NGF | −4.721813392 | 0.043621487 | | | | |
| 35 | cg02069772 | PBXIP1 | −4.69178919 | 0.049219596 | | | | |

Mid-Control

| Hypo Methylated | | | | | Hypermethylated | | | |
|---|---|---|---|---|---|---|---|---|
| Rank | CpG | Gene | T | Adj. P. Val | CpG | Gene | T | Adj. P Val |
| 1 | cg22930808 | PARP9 | −15.03039107 | 1.12E−35 | cg12877361 | OAS1 | 13.82850939 | 5.16E−31 |
| 2 | cg17114584 | IRF7 | −14.83169475 | 4.06E−35 | cg06146977 | EPSTI1 | 11.10677418 | 1.24E−20 |
| 3 | cg21549285 | MX1 | −13.62485489 | 2.77E−30 | cg03100203 | NRIR | 9.792452959 | 4.76E−16 |
| 4 | cg10549986 | RSAD2 | −12.2855965 | 6.83E−25 | cg19020860 | TMPRSS2 | 9.48121171 | 5.53E−15 |
| 5 | cg06981309 | PLSCR1 | −11.6051659 | 2.76E−22 | cg10728454 | GPR176 | 9.375010014 | 1.22E−14 |
| 6 | cg23299102 | | −11.57230475 | 3.17E−22 | cg13409100 | EPSTI1 | 9.151120485 | 6.70E−14 |
| 7 | cg10161996 | | −11.3140783 | 2.76E−21 | cg15999041 | | 9.146554704 | 6.70E−14 |
| 8 | cg03753191 | EPSTI1 | −11.20070162 | 6.65E−21 | cg15627721 | | 8.793586525 | 8.91E−13 |
| 9 | cg24002003 | | −11.15872505 | 8.66E−21 | cg22753522 | | 8.62194708 | 2.91E−12 |
| 10 | cg13155430 | MX1 | −10.50615723 | 2.01E−18 | cg18343506 | PACSIN2 | 8.418359611 | 1.23E−11 |
| 11 | cg07878065 | | −10.47002365 | 2.52E−18 | cg15480653 | FNBP1 | 8.329084695 | 2.16E−11 |
| 12 | cg11251971 | CYSTM1 | 10.44757668 | 2.83E−18 | cg12340267 | ARHGAP26 | 8.023101689 | 1.75E−10 |
| 13 | cg16785077 | MX1 | 10.18162357 | 2.46E−17 | cg04315689 | DGUOK- | 7.921707648 | 3.40E−10 |
| 14 | cg07957619 | GTPBP2 | −9.885810475 | 2.64E−16 | cg06217905 | OAS2 | 7.893080014 | 4.05E−10 |
| 15 | cg26312951 | MX1 | −9.84931198 | 3.22E−16 | cg02183564 | CCDC146 | 7.890002929 | 4.05E−10 |
| 16 | cg22862003 | MX1 | 9.847040377 | 3.22E−16 | cg22488164 | PLBD1 | 7.748069422 | 1.00E−09 |
| 17 | cg00607627 | LAT | 8.963484473 | 2.64E−13 | cg24908179 | DDX60 | 7.718435516 | 1.20E−09 |
| 18 | cg02314339 | | −8.922237037 | 3.48E−13 | cg18518074 | EHD1 | 7.685268515 | 1.47E−09 |
| 19 | cg07362849 | BISPR | −8.688722942 | 1.89E−12 | cg24707889 | ITGB2 | 7.675328398 | 1.54E−09 |
| 20 | cg02650017 | PHOSPHO1 | −8.62833426 | 2.87E−12 | cg03318414 | | 7.495744977 | 5.12E−09 |
| 21 | cg12461141 | TRIM22 | −8.437622903 | 1.10E−11 | cg12685166 | WIPFI | 7.49374813 | 5.12E−09 |
| 22 | cg18543074 | | −8.384263253 | 1.53E−11 | cg16431791 | ADGRE3 | 7.48826958 | 5.21E−09 |
| 23 | cg01028142 | CMPK2 | 8.377359898 | 1.56E−11 | cg10574570 | | 7.442331574 | 6.72E−09 |
| 24 | cg17607231 | SP140 | −8.314452965 | 2.33E−11 | cg27216853 | CYS1 | 7.346937116 | 1.25E−08 |
| 25 | cg03879629 | | −8.048198956 | 1.52E−10 | cg01695994 | | 7.323981499 | 1.43E−08 |
| 26 | cg12439472 | EPSTI1 | −8.046925821 | 1.52E−10 | cg00089960 | SETD1B | 7.303822949 | 1.61E−08 |
| 27 | cg08926253 | IRF7 | −7.990877121 | 2.14E−10 | cg04708790 | OAS1 | 7.24646889 | 2.27E−08 |
| 28 | cg04880620 | OAS2 | 7.831165625 | 5.97E−10 | cg09063556 | CMTM4 | 7.240996943 | 2.31E−08 |
| 29 | cg02138684 | | −7.77096476 | 8.88E−10 | cg12633399 | | 7.22055668 | 2.56E−08 |
| 30 | cg13452062 | IFI44L | −7.7254528 | 1.16E−09 | cg11681597 | SH3BP5L | 7.218260151 | 2.56E−08 |
| 31 | cg24819835 | CD38 | −7.471655061 | 5.66E−09 | cg15425763 | | 7.187788667 | 3.08E−08 |
| 32 | cg08084228 | | −7.470452418 | 5.66E−09 | cg04523589 | CAMP | 7.131754185 | 4.31E−08 |
| 33 | cg21995613 | | −7.282385311 | 1.82E−08 | cg04173586 | DOTIL | 7.016965538 | 8.56E−08 |
| 34 | cg12331471 | SP100 | −7.229929303 | 2.45E−08 | cg20754708 | | 7.010541009 | 8.74E−08 |
| 35 | cg07815522 | PARP9 | −7.174038088 | 3.32E−08 | cg00193210 | SFI1 | 7.009256238 | 8.74E−08 |
| 36 | cg25563198 | FKBP5 | −7.09207715 | 5.50E−08 | cg02909097 | RAPIGAP2 | 6.957637035 | 1.16E−07 |
| 37 | cg03699074 | FAM38A | −7.078970705 | 0.000000059 | cg21805788 | FAM102B | 6.957324901 | 1.16E−07 |
| 38 | cg22984723 | KREMEN1 | −7.076721318 | 5.90E−08 | cg27617715 | FTCDNL1 | 6.949415191 | 1.20E−07 |
| 39 | cg21587506 | IFIT3 | −6.97488403 | 1.08E−07 | cg20389772 | RSAD2 | 6.917209052 | 1.44E−07 |
| 40 | cg05167074 | SHKBP1 | −6.970583342 | 1.09E−07 | cg13937483 | EPSTI1 | 6.858207468 | 2.04E−07 |
| 41 | cg04128669 | | −6.946205216 | 0.000000121 | cg07124045 | KCNIP2 | 6.815836299 | 2.57E−07 |
| 42 | cg25114611 | FKBP5 | −6.873978065 | 1.87E−07 | cg19835618 | TPPP3 | 6.788455469 | 2.98E−07 |
| 43 | cg12230203 | | −6.834995484 | 2.33E−07 | cg01434938 | CDKL1 | 6.759161412 | 3.46E−07 |
| 44 | cg13421019 | PPFIBP2 | −6.820752375 | 2.52E−07 | cg07091793 | | 6.741757851 | 3.72E−07 |
| 45 | cg05455036 | | −6.792963246 | 2.93E−07 | cg23633330 | CD55 | 6.740899642 | 3.72E−07 |
| 46 | cg09342060 | HOXA9 | 6.782723326 | 3.06E−07 | cg14237301 | APOB48R | 6.709090208 | 4.49E−07 |
| 47 | cg12999836 | GRB10 | −6.765772831 | 3.36E−07 | cg25727520 | QPCT | 6.69122541 | 4.91E−07 |
| 48 | cg25998594 | IRF9 | −6.756585101 | 3.48E−07 | cg17615052 | | 6.657123756 | 5.94E−07 |
| 49 | cg24103563 | TRIM34 | −6.745687962 | 3.69E−07 | cg26955963 | LINC00299 | 6.636331857 | 6.62E−07 |
| 50 | cg09971626 | | −6.696972035 | 4.79E−07 | cg10676501 | | 6.622464449 | 7.07E−07 |
| 51 | cg02233071 | RUNX1 | −6.671933729 | 5.47E−07 | cg26296574 | B3GNT8 | 6.608196357 | 7.36E−07 |
| 52 | cg10636246 | AIM2 | −6.637404376 | 6.62E−07 | cg23779886 | | 6.578610078 | 8.74E−07 |
| 53 | cg19371652 | OAS2 | −6.62738387 | 6.93E−07 | cg10446995 | | 6.576510179 | 8.78E−07 |
| 54 | cg02176569 | SP140 | −6.614797626 | 7.33E−07 | cg19060760 | ADIPOR2 | 6.561318235 | 9.46E−07 |
| 55 | cg10447678 | SP110 | −6.611116086 | 7.33E−07 | cg14528056 | GBAP1 | 6.533628711 | 1.11E−06 |
| 56 | cg23837109 | PLAU | 6.610839222 | 7.33E−07 | cg13545732 | EFCAB2 | 6.523829194 | 1.17E−06 |
| 57 | cg08122652 | PARP9 | −6.610358032 | 7.33E−07 | cg06070445 | BCL6 | 6.511167891 | 1.25E−06 |
| 58 | cg22697477 | RUNX1 | −6.562749425 | 9.46E−07 | cg14303526 | ABCC2 | 6.50905169 | 1.26E−06 |
| 59 | cg16486109 | IRF7 | 6.447137283 | 1.74E−06 | cg18712971 | | 6.502600554 | 1.29E−06 |
| 60 | cg03782775 | PTPRA | −6.320814136 | 3.52E−06 | cg06712932 | PLXNC1 | 6.477762946 | 1.48E−06 |
| 61 | cg16462073 | SPNS1 | 6.317951052 | 3.56E−06 | cg04326337 | RPRD1B | 6.477457443 | 1.48E−06 |
| 62 | cg16015295 | RPTOR | −6.302677846 | 0.000003833 | cg20938047 | STXBP6 | 6.454704319 | 1.69E−06 |
| 63 | cg26867393 | GTF2E2 | −6.273439748 | 4.49E−06 | cg26080684 | | 6.450817902 | 1.71E−06 |
| 64 | cg03361738 | ABCA1 | −6.269864545 | 4.55E−06 | cg21071466 | M1AP | 6.424391116 | 1.98E−06 |
| 65 | cg06188083 | IFIT3 | −6.245124527 | 5.22E−06 | cg13570347 | CD44 | 6.412910599 | 2.10E−06 |
| 66 | cg08539067 | GPX1 | −6.238389425 | 5.35E−06 | cg14956920 | VOPP1 | 6.409666199 | 2.12E−06 |
| 67 | cg03607951 | IFI44L | −6.226259398 | 5.67E−06 | cg21718586 | RSAD2 | 6.383820394 | 2.46E−06 |
| 68 | cg26416615 | ARID5B | −6.211790674 | 6.12E−06 | cg18678177 | ARMC9 | 6.340915279 | 3.15E−06 |
| 69 | cg10778971 | IFI27 | −6.190008289 | 6.76E−06 | cg17984982 | EPSTI1 | 6.311603623 | 3.66E−06 |
| 70 | cg06218079 | TBCD | −6.136561813 | 8.69E−06 | cg22107082 | | 6.3004642 | 3.85E−06 |
| 71 | cg13159693 | CD38 | −6.103992319 | 1.02E−05 | cg18519762 | AQP8 | 6.24153493 | 5.30E−06 |
| 72 | cg16400320 | | −6.079048801 | 1.14E−05 | cg05164144 | PDE4D | 6.227553267 | 5.66E−06 |
| 73 | cg26653677 | | −6.039069312 | 1.40E−05 | cg24781413 | | 6.202583624 | 6.42E−06 |
| 74 | cg23540139 | IRF7 | −6.004683302 | 1.67E−05 | cg03921763 | DISC1 | 6.192879699 | 6.74E−06 |
| 75 | cg06679990 | GLI1 | −5.989987715 | 1.75E−05 | cg20610950 | | 6.19012746 | 6.76E−06 |
| 76 | cg11345463 | FAM222A-AS1 | −5.989918163 | 1.75E−05 | cg20866785 | ARHGAP10 | 6.187216253 | 6.78E−06 |
| 77 | cg22323734 | | −5.940208025 | 2.26E−05 | cg17951817 | | 6.18707903 | 6.78E−06 |
| 78 | cg22432956 | | −5.925573245 | 2.41E−05 | cg15551881 | TRAF1 | 6.177578226 | 7.12E−06 |
| 79 | cg01870865 | TREX1 | −5.859978643 | 3.34E−05 | cg00444883 | | 6.165714955 | 7.56E−06 |
| 80 | cg12987761 | USP18 | −5.850510463 | 3.49E−05 | cg11900509 | ANXA11 | 6.164962738 | 7.56E−06 |
| 81 | cg05523603 | | −5.842085305 | 3.60E−05 | cg12439163 | | 6.159206315 | 7.75E−06 |
| 82 | cg19305793 | | −5.831658818 | 3.79E−05 | cg20060108 | IL1RL1 | 6.158543071 | 7.75E−06 |
| 83 | cg18101225 | FGFBP2 | −5.823675344 | 3.89E−05 | cg03172765 | PSMD1 | 6.146018639 | 8.28E−06 |
| 84 | cg09747445 | TLE3 | −5.794100748 | 4.45E−05 | cg07637989 | | 6.124882831 | 9.23E−06 |
| 85 | cg18507060 | OAS3 | −5.785332091 | 4.63E−05 | cg06799784 | C22orf24 | 6.122111571 | 9.32E−06 |
| 86 | cg24181174 | TACC2 | −5.741583464 | 5.62E−05 | cg15184529 | LINC00114 | 6.117858298 | 9.49E−06 |
| 87 | cg22337964 | BISPR | 5.715473019 | 6.37E−05 | cg09018739 | CPNE2 | 6.114671506 | 9.61E−06 |
| 88 | cg13504881 | | −5.687420615 | 7.23E−05 | cg03247739 | CREBBP | 6.102942075 | 1.02E−05 |
| 89 | cg01297684 | | 5.677849394 | 7.49E−05 | cg08319289 | IL1RAP | 6.093540803 | 1.07E−05 |
| 90 | cg03887528 | SP140 | −5.598956663 | 0.000108728 | cg17747635 | | 6.083459252 | 1.12E−05 |
| 91 | cg01553433 | BST2 | −5.587737758 | 0.000115027 | cg24273484 | ROCK1 | 6.078541637 | 1.14E−05 |
| 92 | cg22297448 | D2HGDH | −5.575488003 | 0.000122356 | cg09553840 | ASAP1 | 6.076263033 | 1.15E−05 |
| 93 | cg14353998 | OASL | −5.542304714 | 0.000142144 | cg22052026 | | 6.067586562 | 1.20E−05 |
| 94 | cg00959259 | PARP9 | −5.528921067 | 0.000151382 | cg10722267 | | 6.052269769 | 1.30E−05 |
| 95 | cg11873492 | | −5.52860811 | 0.000151382 | cg25474638 | | 6.021026026 | 1.54E−05 |
| 96 | cg06372654 | IMPDH1 | −5.527656726 | 0.000151382 | cg01153613 | | 6.003812614 | 1.67E−05 |
| 97 | cg06703222 | NFAT5 | 5.4992966 | 0.00017008 | cg15295322 | | 6.00332983 | 1.67E−05 |
| 98 | cg04168577 | PPFIBP2 | −5.476821802 | 0.000188774 | cg08314123 | | 5.999116402 | 1.70E−05 |
| 99 | cg06093152 | | 5.470065801 | 0.000194923 | cg23649619 | TMEM131 | 5.995017772 | 1.73E−05 |
| 100 | cg09803092 | | −5.431017664 | 0.000227014 | cg18217136 | BLCAP | 5.991706887 | 1.75E−05 |

Early Post−Control

| Hypo Methylated | | | | | Hyper Methylated | | | |
|---|---|---|---|---|---|---|---|---|
| Rank | CpG | Gene | T | Adj. P. Val | CpG | Gene | T | Adj. P Val |
| 1 | cg26312951 | MX1 | −1.52E+01 | 2.51E−36 | cg26505274 | OASL | 1.11E+01 | 1.51E−20 |
| 2 | cg13452062 | IFI44L | −1.50E+01 | 5.04E−36 | cg13849515 | MIR3614 | 8.77E+00 | 9.19E−13 |
| 3 | cg06981309 | PLSCR1 | 1.41E+01 | 2.46E−32 | cg07023764 | CCDC26 | 7.81E+00 | 6.30E−10 |
| 4 | cg21549285 | MX1 | −1.30E+01 | 7.32E−28 | cg04213565 | | 7.77E+00 | 7.75E−10 |
| 5 | cg22930808 | PARP9 | −1.30E+01 | 7.32E−28 | cg04987734 | CDC42BPB | 7.63E+00 | 2.08E−09 |
| 6 | cg13155430 | MX1 | −1.25E+01 | 8.34E−26 | cg24644262 | LOC100507391 | 7.46E+00 | 5.84E−09 |
| 7 | cg03607951 | IFI44L | 1.23E+01 | 3.42E−25 | cg07151386 | GMDS | 7.40E+00 | 8.73E−09 |
| 8 | cg12439472 | EPSTI1 | −1.19E+01 | 1.33E−23 | cg09018739 | CPNE2 | 7.33E+00 | 1.32E−08 |
| 9 | cg01028142 | CMPK2 | −1.18E+01 | 2.70E−23 | cg24781413 | | 7.26E+00 | 2.11E−08 |
| 10 | cg08122652 | PARP9 | −1.14E+01 | 1.58E−21 | cg15627721 | | 7.04E+00 | 8.30E−08 |
| 11 | cg05523603 | | −1.13E+01 | 1.79E−21 | cg03172765 | PSMD1 | 7.02E+00 | 9.13E−08 |
| 12 | cg03753191 | EPSTI1 | −1.13E+01 | 2.16E−21 | cg22282161 | DNAH11 | 7.01E+00 | 9.62E−08 |
| 13 | cg07362849 | BISPR | −1.12E+01 | 3.85E−21 | cg26277237 | KANK1 | 7.01E+00 | 9.62E−08 |
| 14 | cg00959259 | PARP9 | −1.08E+01 | 1.19E−19 | cg11900509 | ANXA11 | 6.94E+00 | 1.42E−07 |
| 15 | cg22862003 | MX1 | −1.08E+01 | 1.85E−19 | cg04173586 | DOTIL | 6.94E+00 | 1.44E−07 |
| 16 | cg13304609 | IFI44L | −1.06E+01 | 5.69E−19 | cg06712932 | PLXNC1 | 6.93E+00 | 1.53E−07 |
| 17 | cg10549986 | RSAD2 | −1.04E+01 | 3.06E−18 | cg02183564 | CCDC146 | 6.88E+00 | 1.99E−07 |
| 18 | cg03879629 | | −1.02E+01 | 1.30E−17 | cg13570347 | CD44 | 6.86E+00 | 2.14E−07 |
| 19 | cg07815522 | PARP9 | −1.02E+01 | 1.30E−17 | cg23598089 | ATP2B4 | 6.86E+00 | 2.17E−07 |
| 20 | cg06188083 | IFIT3 | −1.02E+01 | 2.19E−17 | cg02003183 | CDC42BPB | 6.78E+00 | 3.36E−07 |
| 21 | cg12987761 | USP18 | −9.90E+00 | 1.67E−16 | cg13349716 | SERPINE2 | 6.77E+00 | 3.50E−07 |
| 22 | cg24678928 | DDX60 | −9.80E+00 | 3.75E−16 | cg13139542 | | 6.758634339 | 3.68E−07 |
| 23 | cg12331471 | SP100 | −9.66E+00 | 1.13E−15 | cg18518074 | EHD1 | 6.675198845 | 5.99E−07 |
| 24 | cg19371652 | OAS2 | −9.42E+00 | 7.18E−15 | cg15480653 | FNBP1 | 6.66E+00 | 6.43E−07 |
| 25 | cg12013713 | PARP12 | −9.30E+00 | 1.76E−14 | cg12340267 | ARHGAP2 | 6.65E+00 | 6.70E−07 |
| 26 | cg16785077 | MX1 | −9.15E+00 | 5.52E−14 | cg04986899 | XYLT1 | 6.59E+00 | 9.53E−07 |
| 27 | cg04268125 | ADAR | −9.12E+00 | 6.80E−14 | cg01695994 | | 6.56E+00 | 1.17E−06 |
| 28 | cg04880620 | OAS2 | −8.77E+00 | 9.19E−13 | cg12877361 | OAS1 | 6.54E+00 | 1.27E−06 |
| 29 | cg11251971 | CYSTM1 | −8.73E+00 | 1.16E−12 | cg24603130 | ZCCHC2 | 6.50E+00 | 1.62E−06 |
| 30 | cg16400320 | | −8.61E+00 | 2.87E−12 | cg04216721 | ARHGAP18 | 6.47E+00 | 1.88E−06 |
| 31 | cg08926253 | IRF7 | −8.56E+00 | 4.00E−12 | cg17918753 | PRKAG2 | 6.46E+00 | 2.02E−06 |
| 32 | cg05552874 | IFIT1 | −8.49E+00 | 6.41E−12 | cg21805788 | FAM102B | 6.39E+00 | 3.02E−06 |
| 33 | cg05696877 | IFI44L | −8.44E+00 | 9.32E−12 | cg14623715 | PDE7B | 6.38E+00 | 3.09E−06 |
| 34 | cg02314339 | | −8.42E+00 | 1.03E−11 | cg03318414 | | 6.37E+00 | 3.33E−06 |
| 35 | cg10552523 | IFITM1 | −8.33E+00 | 1.91E−11 | cg19796584 | | 6.35E+00 | 3.56E−06 |
| 36 | cg01553433 | BST2 | −8.33E+00 | 1.95E−11 | cg02099683 | FAM53B | 6.34E+00 | 3.66E−06 |
| 37 | cg18543074 | | −8.25E+00 | 3.34E−11 | cg19730422 | CYFIP2 | 6.343194076 | 3.66E−06 |
| 38 | cg05475649 | B2M | −8.20E+00 | 4.76E−11 | cg27612129 | CETP | 6.321808278 | 4.08E−06 |
| 39 | cg21995613 | | −8.09E+00 | 9.73E−11 | cg05263215 | KCNAB2 | 6.28E+00 | 5.10E−06 |
| 40 | cg16644494 | ODF3B | −8.07E+00 | 1.13E−10 | cg10728454 | GPR176 | 6.27E+00 | 5.37E−06 |
| 41 | cg02233071 | RUNX1 | −8.03E+00 | 1.48E−10 | cg00502254 | TNFRSF8 | 6.26E+00 | 5.53E−06 |
| 42 | cg11829870 | KLHDC7B | −7.96E+00 | 2.31E−10 | cg10283551 | TCF12 | 6.26E+00 | 5.61E−06 |
| 43 | cg12906975 | | −7.87E+00 | 4.27E−10 | cg07675998 | | 6.25E+00 | 5.83E−06 |
| 44 | cg01190666 | PRIC285 | −7.85E+00 | 4.83E−10 | cg11956483 | CDK17 | 6.24E+00 | 6.28E−06 |
| 45 | cg09858955 | VRK2 | −7.78E+00 | 7.56E−10 | cg12535090 | NAV2 | 6.20E+00 | 7.70E−06 |
| 46 | cg05883128 | DDX60 | −7.73776994 | 0.000000001 | cg24718773 | HNMT | 6.185069823 | 8.11E−06 |
| 47 | cg25998594 | IRF9 | −7.560739456 | 3.19E−09 | cg00504445 | | 6.14E+00 | 1.02E−05 |
| 48 | cg08084228 | | −7.56E+00 | 3.19E−09 | cg11421485 | CELF2 | 6.14E+00 | 1.04E−05 |
| 49 | cg21979287 | B2M | −7.53E+00 | 3.71E−09 | cg10446995 | | 6.12E+00 | 1.16E−05 |
| 50 | cg02247863 | | −7.46E+00 | 5.77E−09 | cg14640477 | APBB1IP | 6.11E+00 | 1.18E−05 |
| 51 | cg03425812 | B2M | −7.42E+00 | 7.64E−09 | cg10722267 | | 6.102719046 | 1.22E−05 |
| 52 | cg27537252 | B2M | −7.23E+00 | 2.42E−08 | cg11176595 | RHOH | 6.09E+00 | 1.27E−05 |
| 53 | cg06033320 | PDE7A | −7.23E+00 | 2.52E−08 | cg18678177 | ARMC9 | 6.09E+00 | 1.30E−05 |
| 54 | cg17202840 | | −7.04E+00 | 8.32E−08 | cg06130893 | SLC8B1 | 6.077930863 | 0.000013708 |
| 55 | cg12461141 | TRIM22 | −6.93E+00 | 1.53E−07 | cg24707889 | ITGB2 | 6.074230128 | 1.39E−05 |
| 56 | cg10778971 | IFI27 | −6.92E+00 | 1.53E−07 | cg16500036 | | 6.05E+00 | 1.59E−05 |
| 57 | cg04670072 | | −6.87E+00 | 2.06E−07 | cg06810264 | MTURN | 6.04E+00 | 1.70E−05 |
| 58 | cg17114584 | IRF7 | −6.85E+00 | 2.32E−07 | cg14237301 | APOB48R | 6.03E+00 | 1.74E−05 |
| 59 | cg08888522 | IFIH1 | −6.810073465 | 2.90E−07 | cg10705487 | CBY3 | 6.02E+00 | 1.76E−05 |
| 60 | cg05167074 | SHKBP1 | −6.80E+00 | 2.98E−07 | cg04415310 | ZNF664− | 6.017899621 | 1.79E−05 |
| 61 | cg24103563 | TRIM34 | −6.80E+00 | 2.98E−07 | cg05164144 | PDE4D | 6.00E+00 | 1.94E−05 |
| 62 | cg25867318 | STAT3 | −6.76E+00 | 3.68E−07 | cg20625060 | TMEM110 | 5.99E+00 | 2.01E−05 |
| 63 | cg26882438 | PARP14 | −6.76E+00 | 3.68E−07 | cg05195751 | TOX | 5.99E+00 | 2.06E−05 |
| 64 | cg06562969 | EPSTI1 | −6.70E+00 | 5.34E−07 | cg20866785 | ARHGAP10 | 5.98E+00 | 2.18E−05 |
| 65 | cg14943355 | PARP11 | −6.67E+00 | 5.99E−07 | cg00444883 | | 5.97E+00 | 2.23E−05 |
| 66 | cg12828896 | B2M | −6.64E+00 | 7.19E−07 | cg09063556 | CMTM4 | 5.96E+00 | 2.33E−05 |
| 67 | cg11791770 | PHRF1 | −6.48E+00 | 1.76E−06 | cg23089177 | MTURN | 5.93E+00 | 2.67E−05 |
| 68 | cg03258567 | | −6.43E+00 | 2.37E−06 | cg15427587 | TLR4 | 5.88E+00 | 3.41E−05 |
| 69 | cg14870271 | LGALS3BP | −6.37E+00 | 3.32E−06 | cg11186858 | | 5.86E+00 | 3.91E−05 |
| 70 | cg08099136 | PSMB8 | −6.36357461 | 0.000003362 | cg03515040 | DCTN2 | 5.85E+00 | 4.12E−05 |
| 71 | cg00458211 | IFI44L | −6.348895695 | 3.60E−06 | cg04315689 | DGUOK-AS1 | 5.84E+00 | 4.15E−05 |
| 72 | cg07957619 | GTPBP2 | −6.33E+00 | 3.90E−06 | cg05129081 | TP63 | 5.84E+00 | 4.16E−05 |
| 73 | cg09971626 | | −6.29150894 | 4.83E−06 | cg17615052 | | 5.84E+00 | 4.20E−05 |
| 74 | cg01079652 | IFI44 | −6.27E+00 | 5.37E−06 | cg13545732 | EFCAB2 | 5.84E+00 | 4.27E−05 |
| 75 | cg10274453 | | −6.25E+00 | 5.90E−06 | cg20610950 | | 5.82E+00 | 4.53E−05 |
| 76 | cg17986793 | MX1 | −6.21E+00 | 7.10E−06 | cg27216853 | CYS1 | 5.80E+00 | 4.98E−05 |
| 77 | cg20363271 | | −6.19E+00 | 8.11E−06 | cg13693517 | TASP1 | 5.80E+00 | 5.13E−05 |
| 78 | cg07596065 | | −6.18458169 | 8.11E−06 | cg05340191 | | 5.80E+00 | 5.13E−05 |
| 79 | cg08585593 | TYMP | −6.16E+00 | 9.44E−06 | cg18985251 | | 5.79E+00 | 5.20E−05 |
| 80 | cg01680062 | RUNX1 | −6.14E+00 | 1.05E−05 | cg25242306 | KLF12 | 5.79E+00 | 5.34E−05 |
| 81 | cg12999836 | GRB10 | −6.11E+00 | 1.18E−05 | cg24482780 | | 5.78E+00 | 5.54E−05 |
| 82 | cg16411857 | NLRC5 | −6.11E+00 | 1.20E−05 | cg00508575 | ATP2B1 | 5.76E+00 | 6.06E−05 |
| 83 | cg24511258 | TNK2 | 6.07E+00 | 1.39E−05 | cg25594515 | IRF2 | 5.74E+00 | 6.84E−05 |
| 84 | cg14595557 | CMPK2 | −6.03E+00 | 1.71E−05 | cg25656283 | ERCC6 | 5.72E+00 | 7.35E−05 |
| 85 | cg23540139 | IRF7 | −6.03E+00 | 1.74E−05 | cg06200789 | TMEM72 | 5.70E+00 | 8.14E−05 |
| 86 | cg23299102 | | 6.03E+00 | 1.74E−05 | cg07768696 | MSI2 | 5.69E+00 | 8.30E−05 |
| 87 | cg22984723 | KREMEN1 | −6.02E+00 | 1.78E−05 | cg12439163 | | 5.69E+00 | 8.35E−05 |
| 88 | cg18507060 | OAS3 | −6.00E+00 | 1.93E−05 | cg25963583 | MAX | 5.69E+00 | 8.39E−05 |
| 89 | cg22016995 | IRF7 | −5.99E+00 | 2.06E−05 | cg06897548 | TAB1 | 5.68E+00 | 8.57E−05 |
| 90 | cg16292768 | CLU | −5.97E+00 | 2.23E−05 | cg09548275 | NR1H3 | 5.67E+00 | 9.10E−05 |
| 91 | cg01309328 | PSMB8 | −5.96E+00 | 2.35E−05 | cg18455414 | | 5.67E+00 | 9.22E−05 |
| 92 | cg12424383 | | −5.96E+00 | 2.35E−05 | cg01153613 | | 5.629298419 | 0.00011287 |
| 93 | cg26867393 | GTF2E2 | −5.94E+00 | 2.51E−05 | cg16612971 | | 5.62E+00 | 1.17E−04 |
| 94 | cg01297684 | | −5.94E+00 | 2.60E−05 | cg20015729 | UBE2E2 | 5.62E+00 | 1.17E−04 |
| 95 | cg08818207 | TAP1 | −5.909207765 | 0.000030048 | cg05813395 | | 5.606841998 | 0.000125555 |
| 96 | cg01176329 | GABBR1 | −5.89E+00 | 3.36E−05 | cg20938047 | STXBP6 | 5.595087841 | 0.000133111 |
| 97 | cg24166814 | | 5.89E+00 | 3.39E−05 | cg04326337 | RPRD1B | 5.58E+00 | 1.39E−04 |
| 98 | cg11345463 | FAM222A−AS1 | 5.86E+00 | 3.91E−05 | cg21585437 | | 5.57E+00 | 1.48E−04 |
| 99 | cg10959651 | RSAD2 | −5.83E+00 | 4.27E−05 | cg02909097 | RAPIGAP2 | 5.56E+00 | 1.54E−04 |
| 100 | cg14154735 | MOB2 | −5.83E+00 | 4.31E−05 | cg03331035 | MIR30B | 5.56E+00 | 1.54E−04 |

Late−Post Control

| Hypo methylated | | | | | Hyper methylated | | | |
|---|---|---|---|---|---|---|---|---|
| Rank | CpG | Gene | T | Adj. P. Val | CpG | Gene | T | Adj. P Val |
| 1 | cg03607951 | IFI44L | −11.30958055 | 2.29E−20 | cg26505274 | OASL | 7.482925115 | 7.03E−08 |
| 2 | cg13452062 | IFI44L | −8.351229907 | 3.12E−10 | cg01695994 | | 7.384824666 | 1.09E−07 |
| 3 | cg05696877 | IFI44L | 7.900297171 | 5.28E−09 | cg04213565 | | 7.261352928 | 1.77E−07 |
| 4 | cg06981309 | PLSCR1 | −7.260919804 | 1.77E−07 | cg17944885 | | 6.857637772 | 2.04E−06 |
| 5 | cg06033320 | PDE7A | −6.843646195 | 2.04E−06 | cg07675998 | | 6.473929809 | 1.80E−05 |
| 6 | cg26312951 | MX1 | −6.270509856 | 4.34E−05 | cg24297901 | VPS53 | 6.365614703 | 3.14E−05 |
| 7 | cg06188083 | IFIT3 | −6.114830031 | 9.42E−05 | cg22282161 | DNAH11 | 6.310517338 | 3.99E−05 |
| 8 | cg09858955 | VRK2 | −6.071555756 | 0.000113758 | cg23598089 | ATP2B4 | 6.288997559 | 4.19E−05 |
| 9 | cg14864167 | PDE7A | −6.018645454 | 0.000145469 | cg07023764 | CCDC26 | 6.131377436 | 9.13E−05 |
| 10 | cg22298224 | SART1 | 5.842929642 | 0.000371307 | cg08080174 | ZFHX3 | 5.793325584 | 0.000464635 |
| 11 | cg01297684 | | −5.783893784 | 0.00046621 | cg06810264 | MTURN | 5.755609178 | 0.000520173 |
| 12 | cg12331471 | SP100 | −5.614765707 | 0.000913405 | cg09018739 | CPNE2 | 5.744743633 | 0.000528209 |
| 13 | cg03546163 | FKBP5 | −5.490870864 | 0.001533061 | cg13567986 | MTF1 | 5.680683058 | 0.000718762 |
| 14 | cg24678928 | DDX60 | −5.482422169 | 0.001533061 | cg05209514 | CPM | 5.668725631 | 0.000736418 |
| 15 | cg10161996 | | −5.287525705 | 0.003545457 | cg00141498 | CCDC26 | 5.649908783 | 0.000784291 |
| 16 | cg12054453 | TMEM49 | −5.152646649 | 0.006239385 | cg26277237 | KANK1 | 5.580522653 | 0.00105908 |
| 17 | cg12439472 | EPSTI1 | −5.11897478 | 0.007066551 | cg19796584 | | 5.499795761 | 0.001533061 |
| 18 | cg03425812 | B2M | −4.997207602 | 0.011428228 | cg02183564 | CCDC146 | 5.496600343 | 0.001533061 |
| 19 | cg18942579 | TMEM49 | −4.975603314 | 0.012142071 | cg03172765 | PSMD1 | 5.480317627 | 0.001533061 |
| 20 | cg12999836 | GRB10 | −4.973374976 | 0.012142071 | cg19893929 | | 5.375559571 | 0.002509516 |
| 21 | cg09913208 | | −4.911202907 | 0.016133017 | cg12535090 | NAV2 | 5.37434441 | 0.002509516 |
| 22 | cg02111474 | PPFIA1 | −4.881662538 | 0.017543971 | cg04315689 | DGUOK-AS1 | 5.366053886 | 0.002509516 |
| 23 | cg09194657 | TUBGCP2 | −4.879511901 | 0.017543971 | cg13859736 | ST3GAL6 | 5.364560208 | 0.002509516 |
| 24 | cg07573872 | SBNO2 | 4.809772918 | 0.021928864 | cg08796240 | VAC14 | 5.341876978 | 0.002748815 |
| 25 | cg05552874 | IFIT1 | −4.800512981 | 0.021932649 | cg04460609 | LDB2 | 5.264263217 | 0.003803667 |
| 26 | cg26867393 | GTF2E2 | −4.747658644 | 0.025125236 | cg13849515 | MIR3614 | 5.264070883 | 0.003803667 |
| 27 | cg05167074 | SHKBP1 | −4.653287633 | 0.034773178 | cg05263215 | KCNAB2 | 5.17296779 | 0.005897359 |
| 28 | cg19477793 | | −4.637900549 | 0.036914373 | cg07768696 | MSI2 | 5.168402821 | 0.005897359 |
| 29 | cg06520846 | PPCS | −4.625220104 | 0.03869987 | cg14326848 | ZDHHC14 | 5.147333758 | 0.006265923 |
| 30 | cg04621042 | BRD1 | −4.595531209 | 0.041794286 | cg24644262 | LOC100507391 | 5.113944815 | 0.007092408 |
| 31 | cg14775730 | SPEF2 | −4.571594254 | 0.04543211 | cg11421485 | CELF2 | 5.092196872 | 0.007701077 |
| 32 | cg11902329 | | −4.563285938 | 0.046349656 | cg27246571 | HAL | 5.08909217 | 0.007701077 |
| 33 | cg02233071 | RUNX1 | −4.558395724 | 0.04681174 | cg04173586 | DOT1L | 5.014721231 | 0.010803467 |
| 34 | cg26336059 | RAB13 | −4.556128634 | 0.046832854 | cg20318252 | MSI2 | 5.012607095 | 0.010803467 |
| 35 | | | | | cg22584104 | GALNT18 | 4.976093593 | 0.012142071 |
| 36 | | | | | cg16500036 | | 4.90423503 | 0.016392801 |
| 37 | | | | | cg14303526 | ABCC2 | 4.881117231 | 0.017543971 |
| 38 | | | | | cg17825130 | | 4.873370293 | 0.017773442 |
| 39 | | | | | cg17559847 | | 4.869586492 | 0.01780749 |
| 40 | | | | | cg14080094 | | 4.86092749 | 0.018268271 |
| 41 | | | | | cg24603130 | ZCCHC2 | 4.837611364 | 0.01958444 |
| 42 | | | | | cg00690460 | | 4.836958289 | 0.01958444 |
| 43 | | | | | cg15730234 | | 4.836664712 | 0.01958444 |
| 44 | | | | | cg03152456 | FOXN3 | 4.806633313 | 0.021931116 |
| 45 | | | | | cg03333153 | EEFSEC | 4.801445204 | 0.021932649 |
| 46 | | | | | cg09063556 | CMTM4 | 4.787649177 | 0.022983235 |
| 47 | | | | | cg22821289 | | 4.779454642 | 0.023559979 |
| 48 | | | | | cg09548275 | NR1H3 | 4.775280558 | 0.023700206 |
| 49 | | | | | cg12303769 | | 4.767558312 | 0.024248079 |
| 50 | | | | | cg19289950 | HAL | 4.760067553 | 0.024784886 |
| 51 | | | | | cg02378194 | | 4.752603055 | 0.025125236 |
| 52 | | | | | cg24707889 | ITGB2 | 4.750947774 | 0.025125236 |
| 53 | | | | | cg27246361 | | 4.746129101 | 0.025125236 |
| 54 | | | | | cg00508575 | ATP2B1 | 4.730598668 | 0.026687542 |
| 55 | | | | | cg11749142 | MTURN | 4.720315648 | 0.027658573 |
| 56 | | | | | cg09643316 | | 4.705744746 | 0.029246525 |
| 57 | | | | | cg27216853 | CYS1 | 4.695513538 | 0.030306103 |
| 58 | | | | | cg06040542 | BCR | 4.692867841 | 0.030316624 |
| 59 | | | | | cg11847601 | CPNE2 | 4.677175677 | 0.03222798 |
| 60 | | | | | cg13510318 | FAM53B | 4.664965513 | 0.033709494 |
| 61 | | | | | cg03371384 | | 4.659014541 | 0.034253184 |
| 62 | | | | | cg21081878 | HLCS | 4.617384163 | 0.039678672 |
| 63 | | | | | cg21022247 | BIN2 | 4.614211717 | 0.039823465 |
| 64 | | | | | cg21805788 | FAM102B | 4.60715927 | 0.040691069 |
| 65 | | | | | cg12824282 | PTPRM | 4.601085226 | 0.041394738 |
| 66 | | | | | cg15589979 | | 4.594388468 | 0.041794286 |
| 67 | | | | | cg25361236 | | 4.578462773 | 0.044485315 |
| 68 | | | | | cg00841631 | AGK | 4.56276531 | 0.046349656 |

These conclusions were robust to changes in the computational framework used to infer cell proportions (See Appendix FIGS. S5 and S6 of Mao et al., 2023, “A methylation clock model of mild SARS-CoV-2 infection provides insight into immune dysregulation,” Molecular Systems Biology 19 e:11361 which is hereby incorporated by reference). However, the possibility that some of the observed differences correspond to changes in the frequency of cell type not accounted for in computational cell type deconvolution methods cannot be excluded. Comparison of gene expression and methylation levels between the asymptomatic and symptomatic subgroups at each time period showed a maximum of one DEG at false discovery rate (FDR)<0.05, no significant methylation differences, and high correlation between the level of regulation (normalized delta beta values, FIGS. 18B and 18D, and Tables 2.5 and 2.6).

TABLE 2.5
(Differential analysis of methylation levels between the asymptomatic and symptomatic subgroups
at each time period. Raw data-No correction for cell type proportions; uncorrected p-value < 1e−4)
Asymptomatic.Control-Symptomatic.Control

| Hypo methylated | | | | | Hyper methylated | | | |
|---|---|---|---|---|---|---|---|---|
| Rank | CpG | Gene | T | Adj. P. Val | CpG | Gene | | Adj. P. Val |
| 1 | cg23597186 | | −4.570949588 | 4.86E−06 | cg19676502 | | 4.522701655 | 6.11E−06 |
| 2 | cg27333271 | GRM7 | −4.412675321 | 1.02E−05 | cg17081867 | CAPZB | 4.452402058 | 8.49E−06 |
| 3 | cg13088660 | SPTBN1 | −4.245576404 | 2.18E−05 | cg02610856 | HYOU1 | 4.363161569 | 1.28E−05 |
| 4 | cg20278525 | | −4.217539081 | 2.47E−05 | cg26244520 | | 4.211972268 | 2.53E−05 |
| 5 | cg12157782 | | −4.150293714 | 3.32E−05 | cg01357605 | DYNC1LI1 | 4.121351366 | 3.77E−05 |
| 6 | cg07019057 | | −4.133127863 | 0.000035786 | cg26920808 | TBC1D15 | 4.105217847 | 4.04E−05 |
| 7 | cg15073161 | ZBTB17 | −4.102518092 | 4.09E−05 | cg23768829 | | 4.087577153 | 4.36E−05 |
| 8 | cg03081689 | | −4.090415135 | 4.31E−05 | cg16092829 | | 3.910616088 | 0.000092061 |
| 9 | cg05770236 | | −4.052209687 | 5.07E−05 | cg00476149 | HDAC4 | 3.904538075 | 9.44E−05 |
| 10 | cg09895065 | | 4.05123166 | 5.09E−05 | | | | |
| 11 | cg26722522 | PALLD | −4.020175796 | 5.82E−05 | | | | |
| 12 | cg03815093 | | −4.019514434 | 5.83E−05 | | | | |
| 13 | cg25699759 | NTRK3 | −4.015605394 | 5.93E−05 | | | | |
| 14 | cg01708377 | GMDS | −3.986901686 | 6.69E−05 | | | | |
| 15 | cg11185844 | ELN | 3.96460143 | 7.35E−05 | | | | |
| 16 | cg16895216 | KIF25 | −3.959207078 | 0.000075199 | | | | |
| 17 | cg03396152 | NID1 | −3.935062837 | 0.000083175 | | | | |
| 18 | cg19642189 | TP53TG1 | −3.934516617 | 8.34E−05 | | | | |
| 19 | cg05252626 | | −3.933439699 | 8.37E−05 | | | | |
| 20 | cg20684253 | PRKCB | −3.927951761 | 8.57E−05 | | | | |
| 21 | cg11591924 | | −3.922944641 | 8.75E−05 | | | | |
| 22 | cg06470943 | SMAP2 | −3.911476434 | 9.17E−05 | | | | |
| 23 | cg25621991 | | −3.907367518 | 9.33E−05 | | | | |
| 24 | cg07663893 | TNIP3 | −3.90453024 | 9.44E−05 | | | | |

| | | | | |
|---|---|---|---|---|
| 25 | cg02935024 | RAB6B | −3.899132082 | 9.65E−05 |

Asymptomatic.First-Symptomatic.First

| Hypo Methylated | | | | | Hypermethylated | | | |
|---|---|---|---|---|---|---|---|---|
| Rank | CpG | Gene | T | Adj. P. Val | CpG | Gene | T | Adj. P. Val |
| 1 | cg18998363 | SLC5A2 | −3.979748022 | 6.90E−05 | cg08314021 | TSPO | 4.441042196 | 8.95E−06 |
| 2 | cg05068428 | PAK6 | −3.970949033 | 7.16E−05 | cg17273393 | SLC24A5 | 4.130466752 | 3.62E−05 |
| 3 | cg09866743 | ARPP−21 | −3.962125149 | 7.43E−05 | cg04293948 | | 3.943651199 | 8.03E−05 |
| 4 | | | | | cg04903783 | SLC1A3 | 3.912586194 | 0.000091313 |

Asymptomatic.Mid-Symptomatic.Mid

| Hypo Methylated | | | | | Hyper Methylated | | | |
|---|---|---|---|---|---|---|---|---|
| Rank | CpG | Gene | T | Adj. P. Val | CpG | Gene | T | Adj. P. Val |
| 1 | cg09643869 | TBC1D17 | −4.148290488 | 3.35E−05 | cg26049390 | | 4.527319998 | 5.97E−06 |
| 2 | cg18241351 | SCGB1D4 | −4.076785779 | 4.57E−05 | cg11276172 | ITPRIP | 4.135783578 | 3.54E−05 |
| 3 | cg06777259 | DNAI1 | −4.029048289 | 5.60E−05 | cg08266168 | F7 | 4.061438249 | 4.88E−05 |
| 4 | cg20012888 | | −3.970901908 | 7.16E−05 | cg24902139 | TRIM26 | 4.058045977 | 4.95E−05 |
| 5 | cg16248311 | SYT1 | −3.962850164 | 7.41E−05 | cg03251324 | TBC1D22A | 3.960236274 | 7.49E−05 |
| 6 | cg03799727 | POLA2 | −3.929427092 | 8.51E−05 | cg15577390 | CERS4 | 3.957648207 | 7.57E−05 |
| 7 | cg13360758 | TRAPPC12 | 3.906874681 | 9.35E−05 | cg02325544 | | 3.930753276 | 8.47E−05 |

Asymptomatic.Early Post-Symptomatic.EarlyPost

| Hypo methylated | | | | | Hyper methylated | | | |
|---|---|---|---|---|---|---|---|---|
| Rank | CpG | Gene | T | Adj. P. Val | CpG | Gene | T | Adj. P. Val |
| 1 | cg02974539 | | −4.49E+00 | 7.04E−06 | cg05584597 | | 4.45E+00 | 8.77E−06 |
| 2 | cg23531748 | CABLES2 | −4.48E+00 | 7.46E−06 | cg27559724 | RFC4 | 4.42E+00 | 1.01E−05 |
| 3 | cg07509511 | CEP68 | −4.27E+00 | 1.94E−05 | cg21005948 | KIAA0427 | 4.24E+00 | 2.29E−05 |
| 4 | cg09605533 | | −4.15E+00 | 3.31E−05 | cg02886208 | SPON1 | 4.15E+00 | 3.30E−05 |
| 5 | cg20424006 | RAB31 | −4.13E+00 | 3.64E−05 | cg24454932 | | 4.15E+00 | 3.32E−05 |
| 6 | cg11472422 | B3GNTL1 | −4.02E+00 | 5.80E−05 | cg12099356 | | 4.15E+00 | 3.34E−05 |
| 7 | cg13603125 | ARHGEF1 | −3.96E+00 | 7.40E−05 | cg11264356 | UFD1L | 4.11E+00 | 3.88E−05 |
| 8 | cg10623981 | TNFRSF8 | −3.95E+00 | 7.72E−05 | cg11313708 | RPS9 | 4.11E+00 | 3.89E−05 |
| 9 | cg09313177 | TMEM101 | −3.95E+00 | 7.91E−05 | cg02499814 | BAHCCI | 4.08E+00 | 4.53E−05 |
| 10 | cg20248972 | NFKBIL1 | −3.94E+00 | 8.01E−05 | cg03737308 | CDH2 | 4.04E+00 | 5.33E−05 |
| 11 | cg24744210 | SEPT9 | −3.92E+00 | 8.72E−05 | cg25873494 | | 3.95E+00 | 7.70E−05 |
| 12 | cg05835247 | | −3.91E+00 | 9.11E−05 | cg18961533 | RFXANK | 3.94E+00 | 7.98E−05 |
| 13 | cg12936674 | | −3.89E+00 | 9.83E−05 | cg01020987 | Clorf174 | 3.94E+00 | 8.01E−05 |
| 14 | cg26194641 | DHDPSL | −3.89E+00 | 9.94E−05 | cg18391238 | | 3.90E+00 | 9.53E−05 |
| 15 | | | | | cg09635681 | C16orf88 | 3.90E+00 | 9.74E−05 |
| 16 | | | | | cg11250194 | FADS2 | 3.89E+00 | 9.93E−05 |

Asymptomatic.LatePost-Symptomatic.LatePost

| Rank | Hypo methylated | | | | Hyper methylated | | | |
|---|---|---|---|---|---|---|---|---|
| 1 | cg12334842 | WWC2 | −4.544182372 | 5.51E−06 | cg20462883 | BCL7A | 4.580783693 | cg12334842 |
| 2 | cg08900511 | | −4.342579513 | 1.41E−05 | cg10757433 | DAPK1 | 4.525692419 | cg08900511 |
| 3 | cg03686870 | MANBA | −4.209124066 | 2.56E−05 | cg24597234 | BIRC7 | 4.309071653 | cg03686870 |
| 4 | cg07410746 | PIGY | −4.194519333 | 2.73E−05 | cg05805236 | PCNXL3 | 4.307384944 | cg07410746 |
| 5 | cg13691060 | Clorf21 | −4.003210338 | 6.25E−05 | cg20748662 | C10orf32− | 4.196857102 | cg13691060 |
| 6 | cg25305972 | ANKRD44 | −3.957881166 | 7.56E−05 | cg08995542 | NCAPG2 | 4.134242466 | cg25305972 |
| 7 | cg19129145 | | −3.939462425 | 8.17E−05 | cg16514261 | SLC12A5 | 4.036435786 | cg19129145 |
| 8 | cg14516243 | PRDM16 | −3.897174484 | 9.73E−05 | cg07796261 | HMCN1 | 4.006217996 | cg14516243 |
| 9 | | | | | cg11463444 | KIF17 | 4.002400947 | |
| 10 | | | | | cg03351533 | DCP2 | 3.951832292 | |
| 11 | | | | | cg19353449 | | 3.950464066 | |
| 12 | | | | | cg07352185 | | 3.93906817 | |
| 13 | | | | | cg00118369 | GAS7 | 3.931495461 | |
| 14 | | | | | cg05165482 | MALRD1 | 3.923587804 | |
| 15 | | | | | cg00701662 | SIN3B | 3.920407747 | |
| 16 | | | | | cg07777352 | YWHAG | 3.915888107 | |
| 17 | | | | | cg22908882 | RIMBP2 | 3.900939431 | |
| 18 | | | | | cg07442408 | | 3.895332747 | |

TABLE 2.6
(Differential analysis of methylation levels between the asymptomatic and symptomatic subgroups
at each time period. Data were corrected for cell type proportions; uncorrected p-value < 1e−4.)
Asymptomatic.Control-Symptomatic.Control

| Hypo methylated | | | | | Hyper methylated | | | |
|---|---|---|---|---|---|---|---|---|
| Rank | CpG | Gene | T | Adj. P. Val | CpG | Gene | T | Adj. P. Val |
| 1 | cg23597186 | | −5.234260888 | 0.000000258 | cg23768829 | | 4.584695783 | 5.95E−06 |
| 2 | cg22399873 | FBXO41 | −4.477454876 | 9.66E−06 | cg02610856 | HYOU1 | 4.573587855 | 6.26E−06 |
| 3 | cg20684253 | PRKCB | −4.435649725 | 1.16E−05 | cg26244520 | | 4.551411355 | 6.92E−06 |
| 4 | cg26722522 | PALLD | −4.329788612 | 1.85E−05 | cg19676502 | | 4.401993648 | 1.35E−05 |
| 5 | cg07019057 | | −4.320304078 | 1.93E−05 | cg17081867 | CAPZB | 4.397907107 | 1.38E−05 |
| 6 | cg03081689 | | 4.295496765 | 2.15E−05 | cg26920808 | TBC1D15 | 4.258894656 | 2.52E−05 |
| 7 | cg05847835 | | 4.244028474 | 2.68E−05 | cg10824582 | SIK3 | 4.249841208 | 2.62E−05 |
| 8 | cg27333271 | GRM7 | −4.205543879 | 0.000031624 | cg16092829 | | 4.236187593 | 2.78E−05 |
| 9 | cg19989072 | KIAA0040 | −4.197910221 | 3.27E−05 | cg01357605 | DYNC1LI1 | 4.075216144 | 5.46E−05 |
| 10 | cg05770236 | | −4.195369021 | 3.30E−05 | cg08582356 | CA3 | 4.069556681 | 5.59E−05 |
| 11 | cg03396152 | NID1 | −4.186668952 | 3.43E−05 | cg01722297 | SNX11 | 4.068523506 | 5.62E−05 |
| 12 | cg12157782 | | −4.162608388 | 3.79E−05 | cg15846414 | ACIN1 | 4.042468312 | 6.25E−05 |
| 13 | cg16895216 | KIF25 | −4.152793271 | 3.95E−05 | cg00476149 | HDAC4 | 4.039249269 | 6.34E−05 |
| 14 | cg03067929 | RORA | −4.15049688 | 3.99E−05 | cg11783515 | KIAA0355 | 4.032272104 | 6.52E−05 |
| 15 | cg15664462 | MBNL1 | −4.128621443 | 4.37E−05 | cg21940038 | RAPIGAP2 | 4.019567587 | 6.87E−05 |
| 16 | cg25699759 | NTRK3 | −4.12286461 | 4.48E−05 | cg10710951 | | 3.997101423 | 7.53E−05 |
| 17 | cg05252626 | | −4.122402869 | 4.49E−05 | cg14353201 | ALDH3B2 | 3.99640097 | 7.55E−05 |
| 18 | cg11185844 | ELN | −4.117108954 | 4.59E−05 | cg01354572 | GOLGA7 | 3.982996278 | 7.97E−05 |
| 19 | cg17423476 | C6orf99 | −4.11685012 | 4.59E−05 | cg01870976 | PCSK6 | 3.974013881 | 8.27E−05 |
| 20 | cg13088660 | SPTBN1 | −4.113653942 | 0.000046562 | cg14790153 | EHMT2 | 3.95421351 | 8.96E−05 |
| 21 | cg24607725 | ARL6IP4 | −4.110034416 | 4.73E−05 | cg17044705 | | 3.946106452 | 9.26E−05 |
| 22 | cg17390821 | TAB2 | 4.105436804 | 4.82E−05 | | | | |
| 23 | cg06470943 | SMAP2 | −4.100843817 | 4.91E−05 | | | | |
| 24 | cg02935024 | RAB6B | −4.072439594 | 5.53E−05 | | | | |
| 25 | cg23887832 | NSUN4 | −4.068466545 | 0.000056172 | | | | |
| 26 | cg26467809 | ATG7 | −4.060649934 | 0.000058015 | | | | |
| 27 | cg20278525 | | 4.055906602 | 5.92E−05 | | | | |
| 28 | cg07148467 | COL27A1 | −4.047623145 | 6.12E−05 | | | | |
| 29 | cg19587207 | | −4.045158792 | 6.18E−05 | | | | |
| 30 | cg15073161 | ZBTB17 | −4.028718561 | 6.62E−05 | | | | |
| 31 | cg16637899 | CASP14 | −4.025375433 | 6.71E−05 | | | | |
| 32 | cg07536920 | RORB | −3.997208775 | 7.52E−05 | | | | |
| 33 | cg24932312 | | −3.966440245 | 8.53E−05 | | | | |
| 34 | cg26234786 | FILIP1L | 3.964229756 | 8.60E−05 | | | | |
| 35 | cg19642189 | TP53TG1 | −3.961146103 | 0.000087106 | | | | |
| 36 | cg23340936 | GOLIM4 | −3.938550722 | 9.54E−05 | | | | |
| 37 | cg21952077 | CDH4 | −3.928615804 | 0.000099303 | | | | |

Asymptomatic.First-Symptomatic.First

| Hypo Methylated | | | | | Hypermethylated | | | |
|---|---|---|---|---|---|---|---|---|
| Rank | CpG | Gene | T | Adj. P. Val | CpG | Gene | T | Adj. P. Val |
| 1 | | | | | cg17357892 | | 4.773869907 | 2.47E−06 |
| 2 | | | | | cg08314021 | TSPO | 4.619759989 | 5.06E−06 |
| 3 | | | | | cg04903783 | SLC1A3 | 4.580242143 | 6.07E−06 |
| 4 | | | | | cg17273393 | SLC24A5 | 4.261258948 | 2.49E−05 |
| 5 | | | | | cg11236543 | | 4.159550089 | 3.84E−05 |
| 6 | | | | | cg20963261 | TMEM232 | 4.142019574 | 0.000041352 |
| 7 | | | | | cg13538165 | LOC101929705 | 4.137920491 | 0.000042069 |
| 8 | | | | | cg21910650 | MEA1 | 4.109234036 | 4.74E−05 |
| 9 | | | | | cg04293948 | | 4.100745593 | 4.91E−05 |
| 10 | | | | | cg12510708 | NFE2L3 | 4.097632498 | 4.98E−05 |
| 11 | | | | | cg11859249 | TSTA3 | 4.086688254 | 5.21E−05 |
| 12 | | | | | cg02406032 | NFATC1 | 4.081631238 | 5.32E−05 |
| 13 | | | | | cg08642992 | LOC100507073 | 3.974886014 | 8.24E−05 |
| 14 | | | | | cg05366063 | DPYSL3 | 3.95711564 | 8.85E−05 |
| 15 | | | | | cg04747146 | TCERG1L | 3.952227234 | 9.03E−05 |
| 16 | | | | | cg26802307 | VWCE | 3.94077485 | 0.000094566 |
| 17 | | | | | cg16486198 | SV2C | 3.939228751 | 9.52E−05 |

Asymptomatic.Mid-Symptomatic.Mid

| Hypo Methylated | | | | | Hyper Methylated | | | |
|---|---|---|---|---|---|---|---|---|
| Rank | CpG | Gene | T | Adj. P. Val | CpG | Gene | T | Adj. P. Val |
| 1 | cg11769773 | KAZN | 4.235454099 | 2.78E−05 | cg26049390 | | 4.700142949 | 3.49E−06 |
| 2 | cg18241351 | SCGB1D4 | −4.232393156 | 2.82E−05 | cg24902139 | TRIM26 | 4.4241501 | 1.22E−05 |
| 3 | cg09643869 | TBC1D17 | −4.188601319 | 3.40E−05 | cg11276172 | ITPRIP | 4.257103931 | 2.54E−05 |
| 4 | cg03990588 | TMEM150 | −4.109408057 | 4.74E−05 | cg02325544 | | 4.202977797 | 3.20E−05 |
| 5 | cg01874407 | FAM19A5 | −4.034685368 | 6.46E−05 | cg19063834 | FAM134B | 4.199949232 | 3.24E−05 |
| 6 | cg12361951 | SRC | −3.991090394 | 7.71E−05 | cg08111629 | | 4.196577681 | 3.29E−05 |
| 7 | cg06777259 | DNAI1 | 3.961001458 | 8.72E−05 | cg00332855 | NEXN | 4.12615802 | 4.42E−05 |
| 8 | cg20500126 | | −3.95768851 | 8.83E−05 | cg21219191 | | 4.073740197 | 5.50E−05 |
| 9 | cg20012888 | | −3.949727643 | 9.12E−05 | cg11726363 | C19orf28 | 4.067654854 | 5.64E−05 |
| 10 | cg09089187 | KCNIP1 | 3.927405517 | 9.98E−05 | cg03251324 | TBC1D22A | 4.067049461 | 5.65E−05 |
| 11 | | | | | cg27084712 | | 4.056187638 | 5.91E−05 |
| 12 | | | | | cg03224572 | GDF6 | 4.044265139 | 6.21E−05 |
| 13 | | | | | cg08266168 | F7 | 4.019587143 | 6.87E−05 |
| 14 | | | | | cg15800530 | ZP3 | 4.003943574 | 7.32E−05 |
| 15 | | | | | cg16811073 | | 3.963994569 | 8.61E−05 |
| 16 | | | | | cg23732147 | | 3.953328965 | 8.99E−05 |
| 17 | | | | | cg19856127 | NTM | 3.949944353 | 9.11E−05 |
| 18 | | | | | cg15577390 | CERS4 | 3.941809399 | 9.42E−05 |
| 19 | | | | | cg15922705 | COL9A1 | 3.935712274 | 9.65E−05 |

Asymptomatic.Early Post-Symptomatic.EarlyPost

| Hypo methylated | | | | | Hyper methylated | | | |
|---|---|---|---|---|---|---|---|---|
| Rank | CpG | Gene | T | Adj. P. Val | CpG | Gene | T | Adj. P. Val |
| 1 | cg23531748 | CABLES2 | −4.90E+00 | 1.33E−06 | cg21243889 | | 4.48E+00 | 9.39E−06 |
| 2 | cg14485787 | MGMT | −4.36E+00 | 1.62E−05 | cg27559724 | RFC4 | 4.38E+00 | 1.48E−05 |
| 3 | cg04923604 | NFATC2 | −4.33E+00 | 1.85E−05 | cg05584597 | | 4.35E+00 | 1.68E−05 |
| 4 | cg02974539 | | −4.32E+00 | 1.89E−05 | cg02499814 | BAHCC1 | 4.35E+00 | 1.73E−05 |
| 5 | cg11640253 | UPF1 | −4.26E+00 | 2.53E−05 | cg21005948 | KIAA0427 | 4.25E+00 | 2.61E−05 |
| 6 | cg09313177 | TMEM101 | −4.22E+00 | 3.03E−05 | cg11264356 | UFD1L | 4.14E+00 | 4.09E−05 |
| 7 | cg13603125 | ARHGEF1 | −4.16E+00 | 3.81E−05 | cg11301879 | | 4.14E+00 | 4.09E−05 |
| 8 | cg27474643 | EML3 | −4.06E+00 | 5.81E−05 | cg09635681 | C16orf88 | 4.13E+00 | 4.26E−05 |
| 9 | cg07509511 | CEP68 | −4.06E+00 | 5.85E−05 | cg23726427 | GLI2 | 4.04E+00 | 6.26E−05 |
| 10 | cg08187687 | | −4.03E+00 | 6.58E−05 | cg18758900 | | 4.01E+00 | 7.07E−05 |
| 11 | cg20248972 | NFKBIL1 | −4.03E+00 | 6.68E−05 | cg12099356 | | 3.98E+00 | 8.10E−05 |
| 12 | cg01803461 | | −4.03E+00 | 6.71E−05 | cg03737308 | CDH2 | 3.96E+00 | 8.85E−05 |
| 13 | cg01387179 | MVB12B | −4.02E+00 | 6.89E−05 | cg23109206 | ANKRD55 | 3.95E+00 | 9.15E−05 |
| 14 | cg04370834 | MAP2K3 | −4.01E+00 | 7.18E−05 | | | | |
| 15 | cg27586797 | | −4.01E+00 | 7.22E−05 | | | | |
| 16 | cg05835247 | | −4.00E+00 | 7.31E−05 | | | | |
| 17 | cg12283439 | | −3.99E+00 | 7.62E−05 | | | | |
| 18 | cg22459236 | LRIG3 | −3.99E+00 | 7.76E−05 | | | | |
| 19 | cg24281697 | KIAA0564 | −3.99E+00 | 7.90E−05 | | | | |
| 20 | cg09847584 | RIMBP2 | −3.95E+00 | 9.02E−05 | | | | |
| 21 | cg10623981 | TNFRSF8 | −3.95E+00 | 9.11E−05 | | | | |
| 22 | cg10764559 | PARVA | −3.94E+00 | 9.43E−05 | | | | |
| 23 | cg17788082 | PCNXL2 | −3.93E+00 | 9.69E−05 | | | | |

Asymptomatic.LatePost-Symptomatic.LatePost

| Rank | Hypo methylated | | | | Hyper methylated | | | |
|---|---|---|---|---|---|---|---|---|
| 1 | cg08900511 | | −5.037076562 | 6.93E−07 | cg20462883 | BCL7A | 4.876208208 | 1.52E−06 |
| 2 | cg12334842 | WWC2 | −4.72914188 | 3.05E−06 | cg10757433 | DAPK1 | 4.64518878 | 4.51E−06 |
| 3 | cg19129145 | | −4.338159004 | 1.79E−05 | cg05805236 | PCNXL3 | 4.438418733 | 1.15E−05 |
| 4 | cg05057720 | CLEC14A | −4.333330322 | 1.82E−05 | cg01923089 | | 4.348356384 | 1.71E−05 |
| 5 | cg07410746 | PIGY | −4.295239358 | 2.15E−05 | cg24597234 | BIRC7 | 4.308530835 | 2.03E−05 |
| 6 | cg03686870 | MANBA | 4.181334222 | 3.50E−05 | cg15202848 | | 4.284790494 | 2.25E−05 |
| 7 | cg23257604 | TNXB | −4.131135983 | 4.33E−05 | cg20748662 | C10orf32- | 4.25040673 | 2.61E−05 |
| 8 | cg13691060 | Clorf21 | −4.029125643 | 6.60E−05 | cg21179831 | BAT1 | 4.236374712 | 2.77E−05 |
| 9 | cg15930120 | AKAP6 | −4.014113588 | 7.02E−05 | cg23199938 | | 4.190183842 | 3.38E−05 |
| 10 | cg04799330 | | −4.006416647 | 7.25E−05 | cg05165482 | MALRD1 | 4.124991756 | 4.44E−05 |
| 11 | cg13046164 | UNC50 | −3.952073152 | 9.04E−05 | cg24989739 | POPDC3 | 4.122655567 | 4.48E−05 |
| 12 | | | | | cg11463444 | KIF17 | 4.053881635 | 0.000059657 |
| 13 | | | | | cg08995542 | NCAPG2 | 4.04258329 | 0.000062497 |
| 14 | | | | | cg22908882 | RIMBP2 | 4.00394673 | 7.32E−05 |
| 15 | | | | | cg03351533 | DCP2 | 3.998929541 | 7.47E−05 |
| 16 | | | | | cg07796261 | HMCN1 | 3.979883502 | 0.00008074 |
| 17 | | | | | cg14262357 | | 3.974631689 | 8.25E−05 |
| 18 | | | | | cg16514261 | SLC12A5 | 3.970553291 | 8.39E−05 |
| 19 | | | | | cg14792595 | | 3.9455737 | 0.000092756 |
| 20 | | | | | cg07777352 | YWHAG | 3.942114724 | 9.41E−05 |

Because the molecular responses following mildly symptomatic and asymptomatic infections in this cohort were indistinguishable, these groups were combined for all subsequent analyses. The changes of the genes and methylation sites that were significantly altered at Mid compared to Control were examined. When these gene and methylation levels were plotted at all time periods, the genes overlapped with Control levels following clearance of the virus (FIG. 1D of Mao et al., 2023, “A methylation clock model of mild SARS-CoV-2 infection provides insight into immune dysregulation,” Molecular Systems Biology 19 e:11361 which is hereby incorporated by reference and FIGS. 18C-18F). In contrast, the methylation changes were more prolonged both for sites associated with DEG and for sites not associated with (FIGS. 18C-18F).

### Methylation Site Dynamics

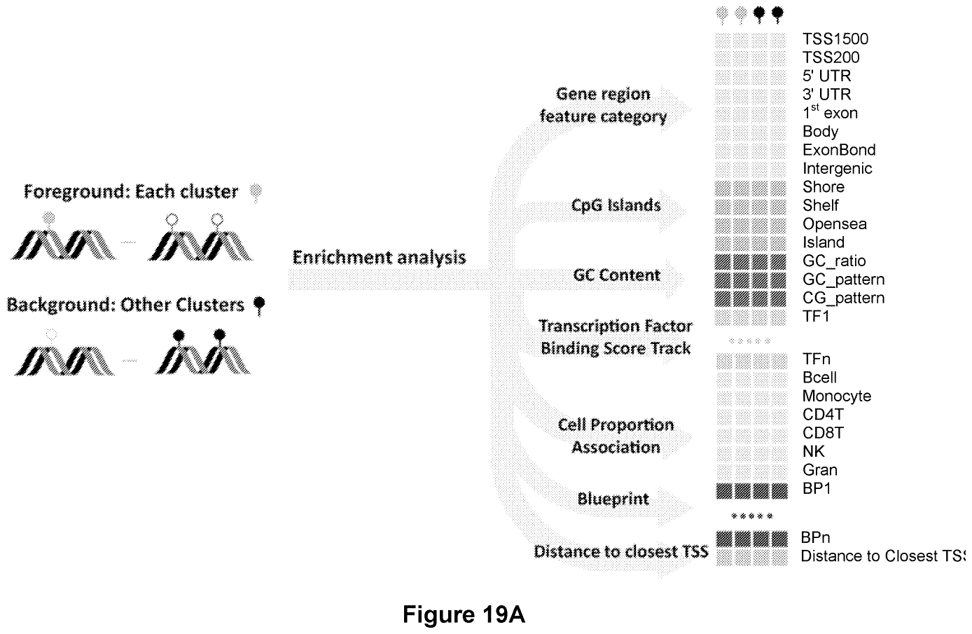

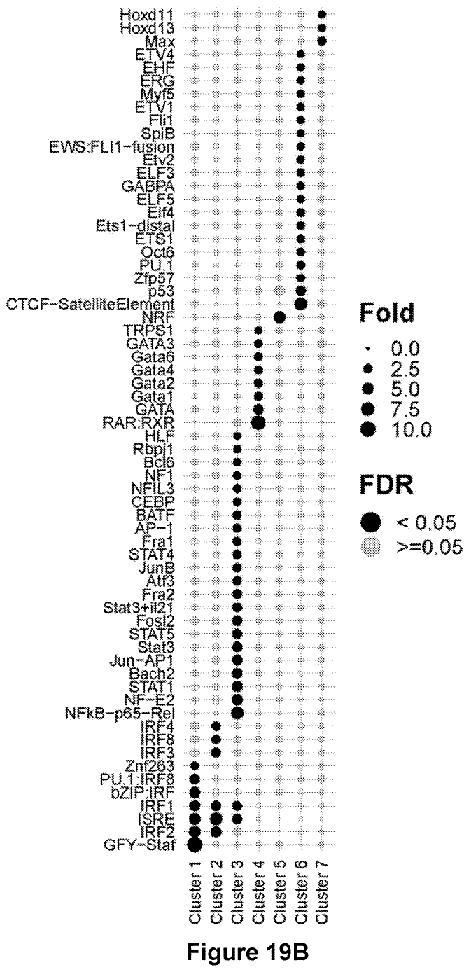

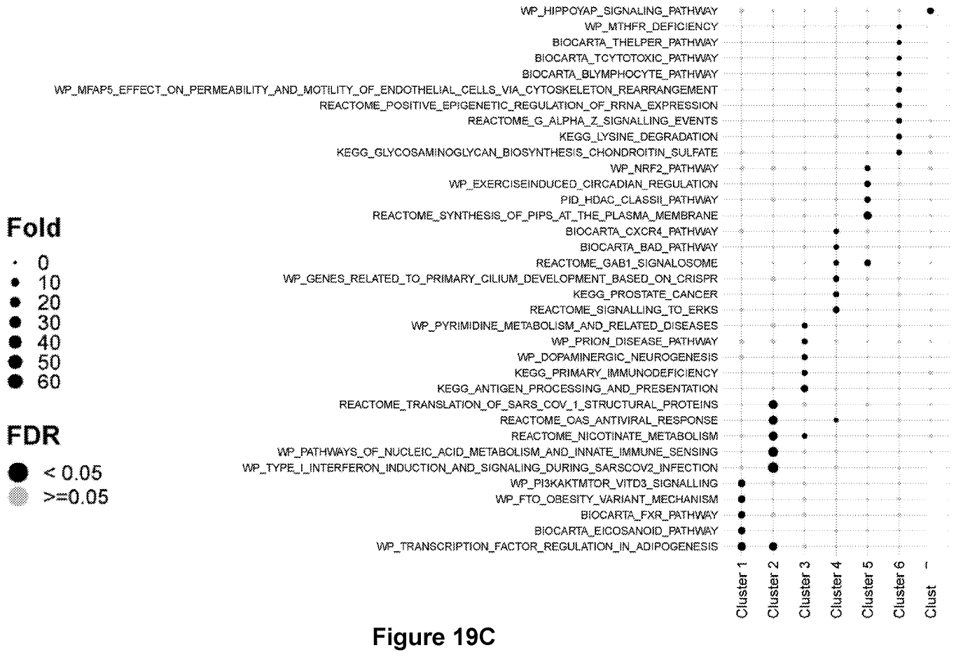

When the methylation levels of all DMS were aligned by day relative to the initial PCR-positive test and clustered hierarchically using dynamic time-warping distance, three hypomethylation (Clusters 1-3) and 4 hypermethylation (Clusters 4-7) trajectories were observed (FIG. 2A of Mao et al., 2023, “A methylation clock model of mild SARS-CoV-2 infection provides insight into immune dysregulation,” Molecular Systems Biology 19 e:11361 which is hereby incorporated by reference). To evaluate whether the clusters distinguished by time trajectories could reflect different mechanisms, enrichment was assessed for various properties (See FIG. 19A) including: nearby transcription factor binding sites (TFBS), pathways, Blueprint Epigenome project cell type signatures (Stunnenberg et al, 2016), cell type proportions, association with single cell sequencing-derived cell type markers, CpG island categories, gene region feature categories, CG/GC content, and distance to transcription start site (See FIG. EV3B through EVB3-I of Mao et al., 2023, “A methylation clock model of mild SARS-CoV-2 infection provides insight into immune dysregulation,” Molecular Systems Biology 19 e:11361 which is hereby incorporated by reference). When the 200-bp regions centered on the DMS in each cluster were analyzed for TFBS enrichment using the HOMER motif database (Duttke et al., 2019), each of the three hypomethylation clusters and three of the four hypermethylation clusters showed enrichment of distinct TFBS for each cluster (FIG. 19B). It was found that the DMS in each cluster were enriched in Blueprint cell type markers (See FIG. EV3B of Mao et al., Id. which is hereby incorporated by reference). Among the hypomethylated clusters, early changes were generally associated with myeloid cell signatures and later changes with mature lymphocytes (See FIG. EV3B of Mao et al., Id. which is hereby incorporated by reference). Cluster 3, which contained sites showing prolonged hypomethylation, was enriched in mature B cell lineage signatures, including plasma and germinal center cells (See FIG. EV3B of Mao et al., Id. which is hereby incorporated by reference). This finding was concordant with the TFBS enrichment analysis, which showed the association of Cluster 3 with the germinal center regulator BCL6 (FIG. 19B). In addition, the genes annotated to the DMS in each dynamical cluster were enriched for specific MSigDB canonical (Liberzon et al, 2011) and hallmark (Liberzon et al, 2015) pathways (FIG. 19C). These findings indicate that the temporal dynamics clusters are biologically coherent, and suggests that the regulation of DMS within each cluster involves activation of different pathways and relies on distinct sets of transcription factors that contribute to the targeting of the methylation regulatory machinery.

### SARS-CoV-2 Methylation Clock

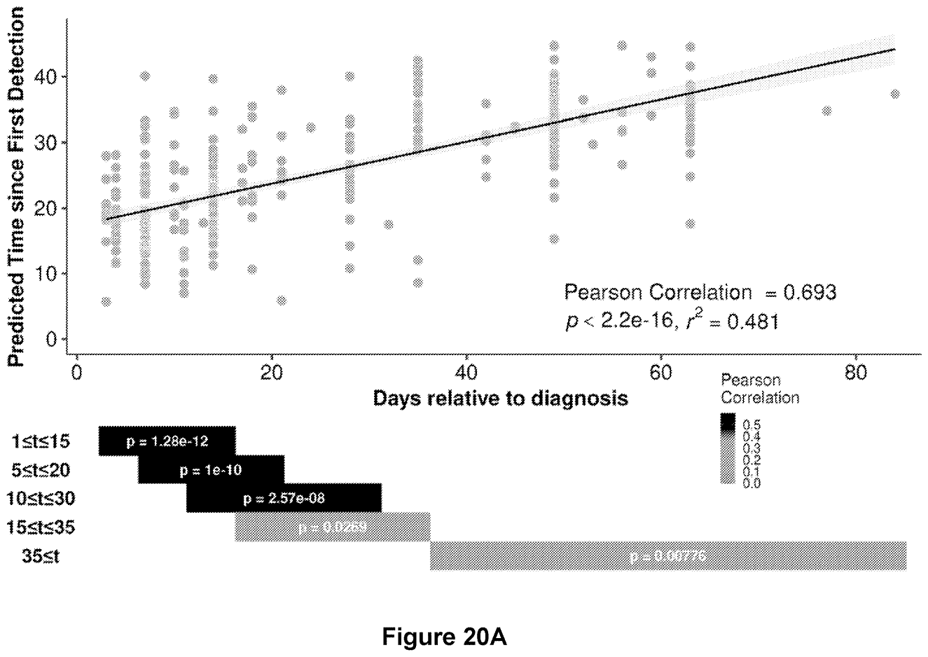

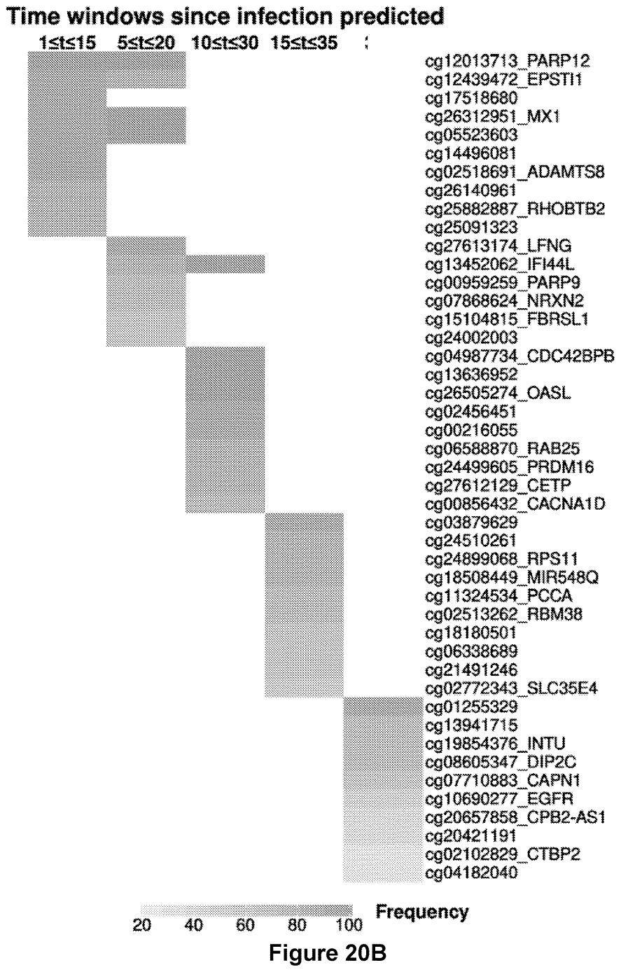

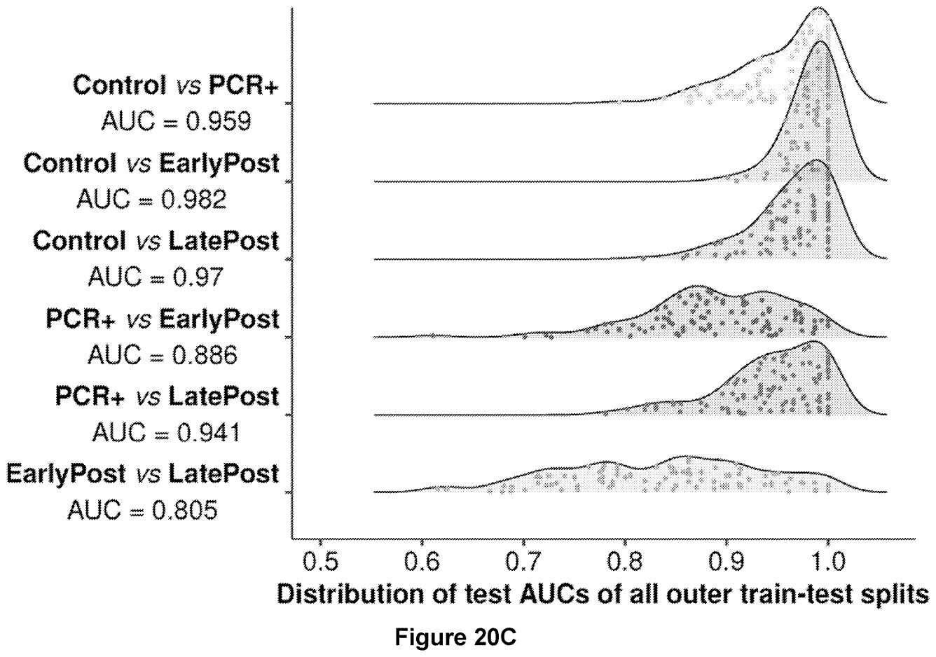

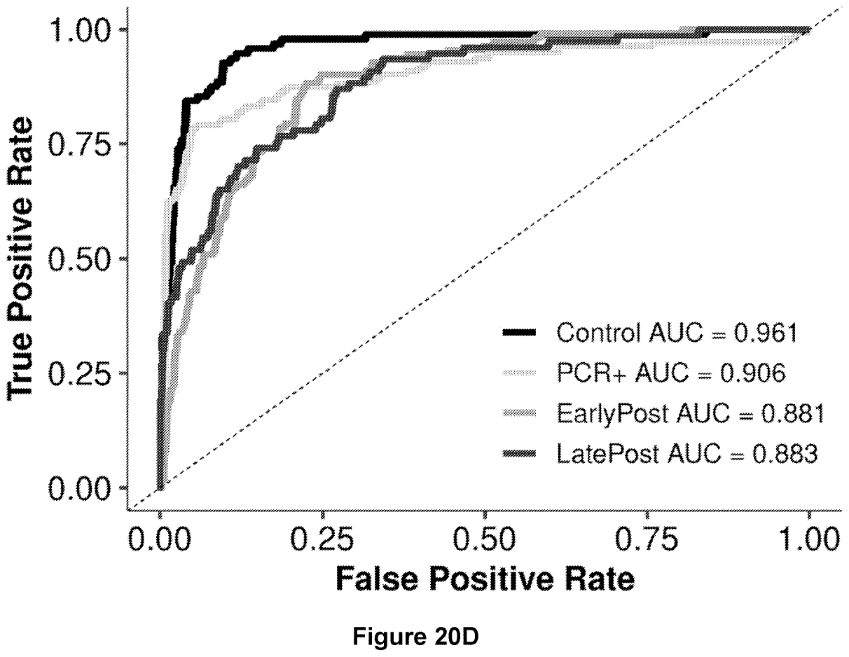

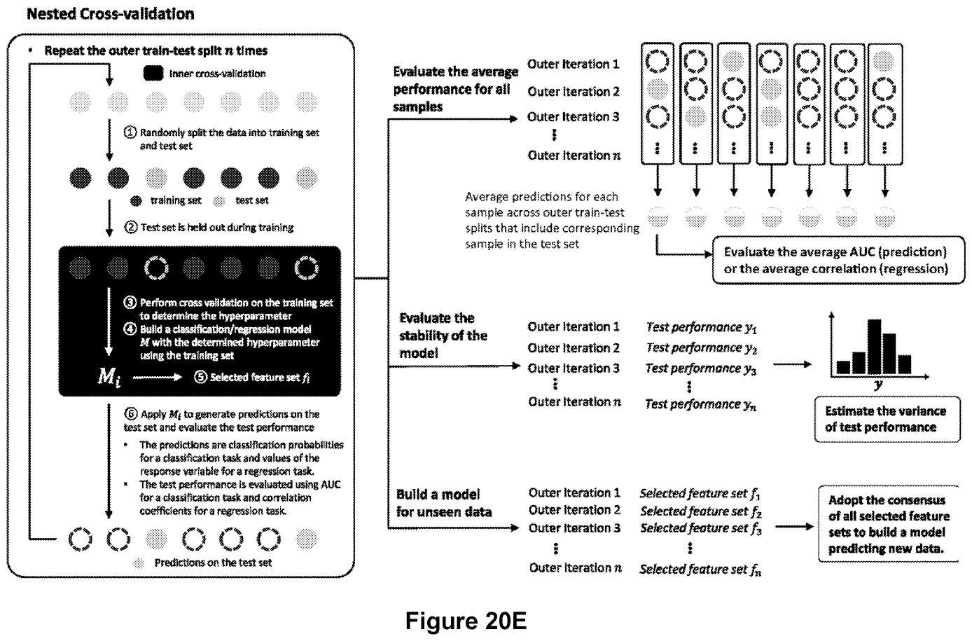

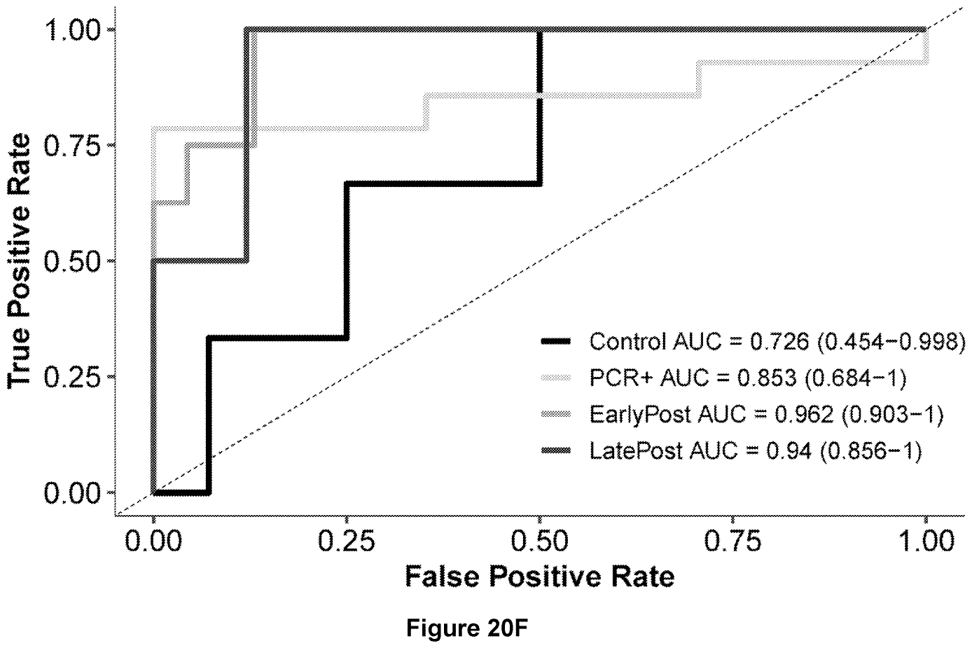

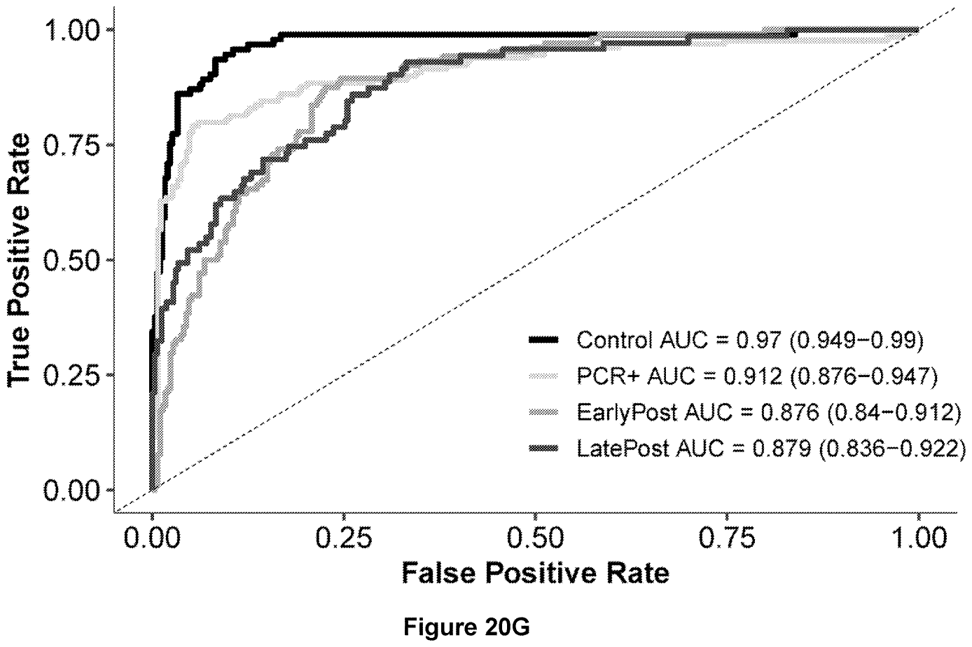

The potential for DNA methylation dynamics to predict time since infection was investigated. A nested cross-validation procedure was used to generate an elastic net regression model trained on the methylation data to predict day since infection. The training procedure for modeling in accordance with one embodiment of the present disclosure is shown schematically in FIG. 20E. Model predictions were highly correlated with the actual day since infection (FIG. 20A). To examine the accuracy of methylation-based prediction over time and to determine the sites most important for predictions at different post-infection periods, separate models were trained on all CpG sites for samples from different time windows, and sites that were most often selected by 100 model iterations for each window were determined. The models showed predictive power for all five time windows examined (FIG. 20A). The most important methylation sites for predicting different time windows showed little overlap, indicating that the methylation patterns continue to evolve months after the initial infection (FIG. 20B). The accuracy of binary classification models to distinguish between pairs of Control, PCR-positive, EarlyPost, and LatePost periods was examined (FIG. 20C). The models for distinguishing pre-infection and post-infection groups showed the highest accuracy, and all iterations for all classification problems performed above chance. A multi-class classifier was constructed that assigned each sample to its time period with high accuracy, ranging from an area under the receiver-operator curve (AUC) of 0.88 for the two Post periods to 0.96 for Control (FIG. 20D). One limitation of this analysis is that most participants were male. To determine whether these analyses were applicable to females, the multiclass classifier performance in 31 samples from 11 female participants (FIG. 20F) and in 397 samples from 122 male participants (FIG. 20G) was compared. Overall, the samples from both sexes were classified with similar accuracy, supporting the relevance of the model for both sexes.

### Relationship to Other Conditions

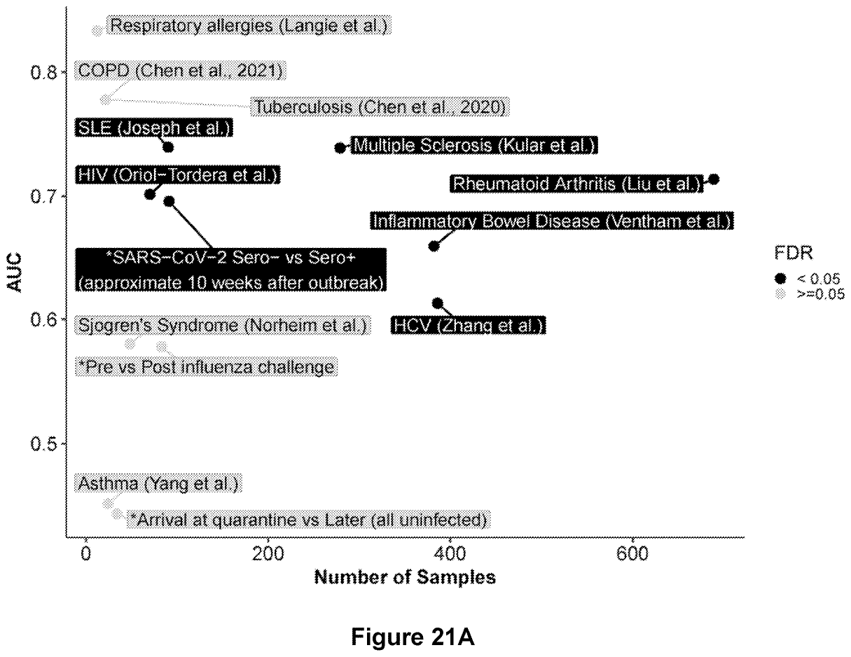

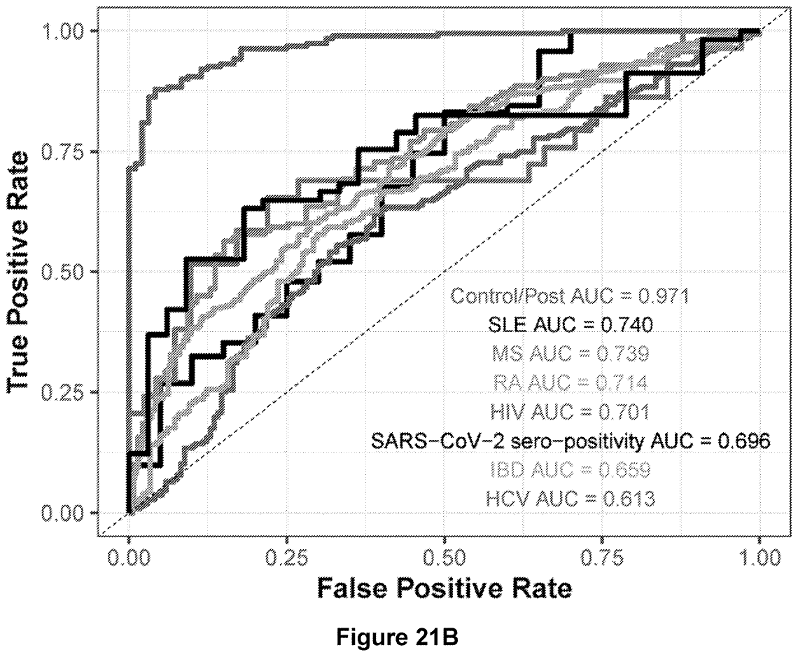

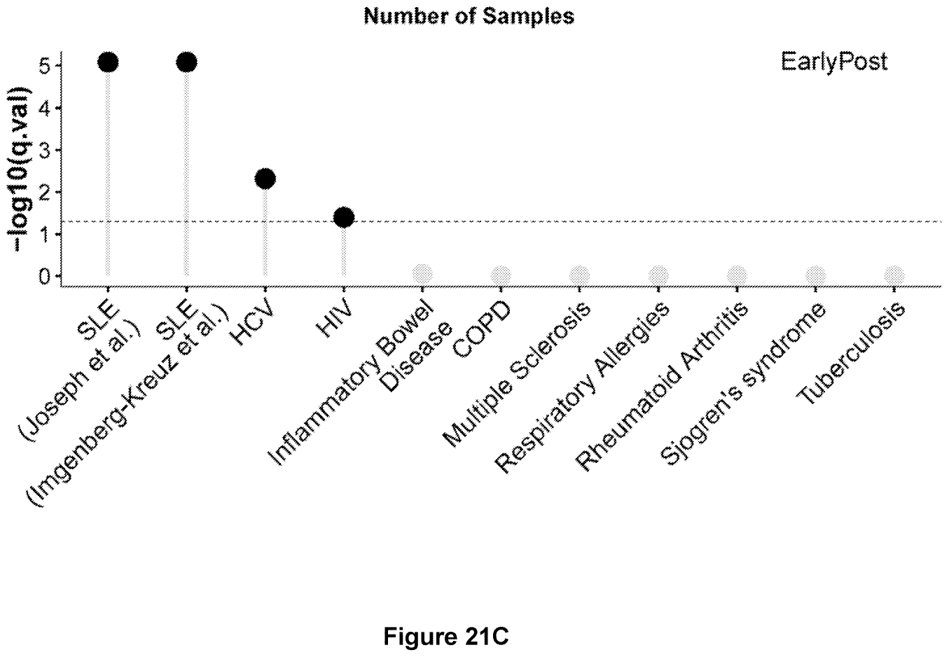

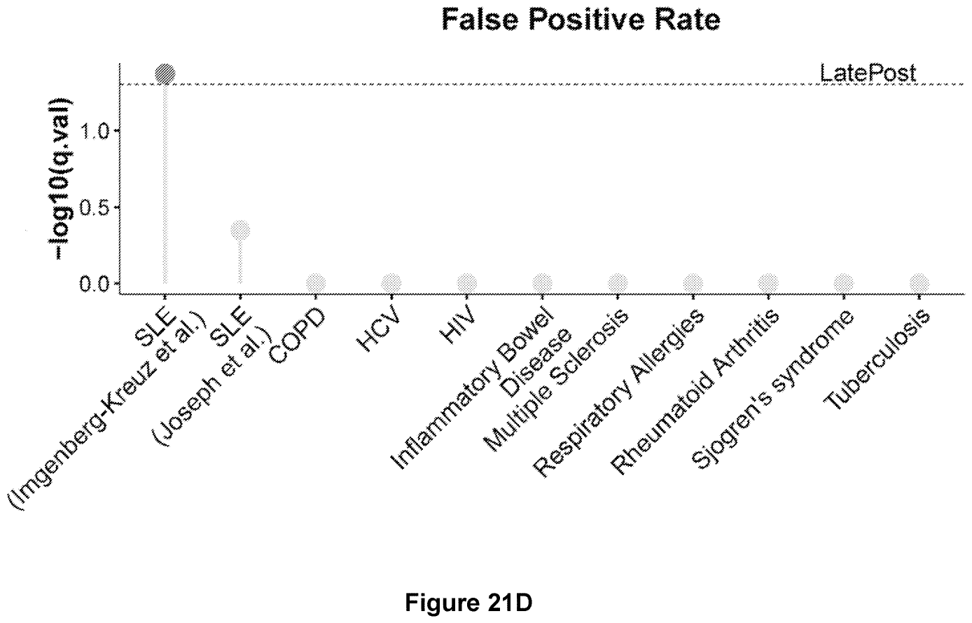

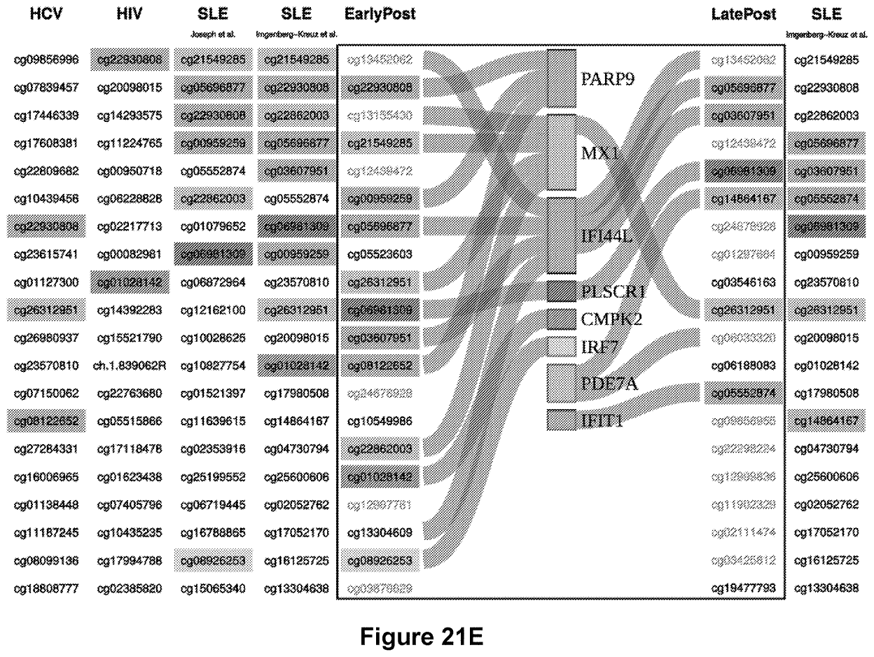

A determination was made as whether a model trained to distinguish post PCR+ samples (EarlyPost and LatePost combined) from Control could also distinguish other conditions associated with altered immunological states. Between mid-April and mid-May 2020, an outbreak of SARS-CoV-2 occurred in several companies during basic training at Parris Island, South Carolina. Although few cases were confirmed by PCR testing, a retrospective serological study of exposed recruits was performed (Sah et al, 2021). Using DNA methylation from samples obtained in mid-July, 2020 about 10 weeks after exposure, from 71 seropositive and 20 seronegative recruits, the model assignment of Control and post PCR+ correlated with serological status (receiver operator curve AUC=0.7, FDR=0.016; FIGS. 21A and 21B and 21E). This indicates that seropositive and seronegative recruits who were exposed to SARS-CoV-2 can be distinguished retrospectively by their methylation states. Most of the infected recruits in the longitudinal study were first PCR-positive following the two-week supervised quarantine and the first few weeks of basic training. Using longitudinal samples from recruits who remained PCR-negative as a time of training control study, the present disclosure found that the model did not distinguish the quarantine and basic training samples (FIG. 21A).

The classification of samples from infections and inflammatory diseases (FIG. 21F) was examined. It was found that the model did not distinguish samples from before 4 weeks after H3N2 influenza challenge (FIG. 21A). See also datasets EV9 and EV10 of Mao et al., 2023, “A methylation clock model of mild SARS-CoV-2 infection provides insight into immune dysregulation,” Molecular Systems Biology 19 e:11361 which is hereby incorporated by reference). Significant classification accuracy was obtained in distinguishing control samples in each dataset from systemic lupus erythematosus (SLE), multiple sclerosis, chronic hepatitis C virus infection, rheumatoid arthritis, inflammatory bowel disease and hepatitis C virus infection, as well as for high versus low levels of chronic human immunodeficiency virus infections (FIGS. 21A-21B). Significant accuracy was not achieved for classifying asthma, Sjogren's syndrome, respiratory allergies, tuberculosis infection and chronic obstructive pulmonary disease (FIG. 21A). To further examine the relationship of the post infection methylation state induced by SARS-CoV-2 to that associated with other diseases, a determination of the enrichment of post infection DMS in the CHARM study to those reported in studies of other diseases was made. Significant enrichment was observed between EarlyPost period DMS and the HCV study, an HIV study and two SLE studies (FIG. 21C). The LatePost DMS were significantly enriched in one of the two SLE studies (FIG. 21D). Comparing the post infection SARS-CoV-2 DMS and the studies showing enrichment by order of significance of DMS showed a high overlap between the DNA hypomethylation sites in SARS-CoV-2 and those in SLE (FIG. 21E). Seven of the eight most significant EarlyPost DMS that were assayed in either of two SLE datasets, were included in the top 10 DMS identified in the SLE methylation studies, and six of the most significant LatePost DMS were among the 14 most significant sites identified in one of the SLE studies (FIG. 21E).

Overall, the methylation model has considerable overlap with other inflammatory conditions including chronic infection and autoimmune diseases and is most similar to SLE. This is consistent with the observation that the changes we observe are related to the modulation of interferon signaling, which is activated in SLE (Ronnblom & Leonard, 2019).

### Immunological Effects of Prolonged Methylation Pattern and Relevance to a More Diverse Cohort.

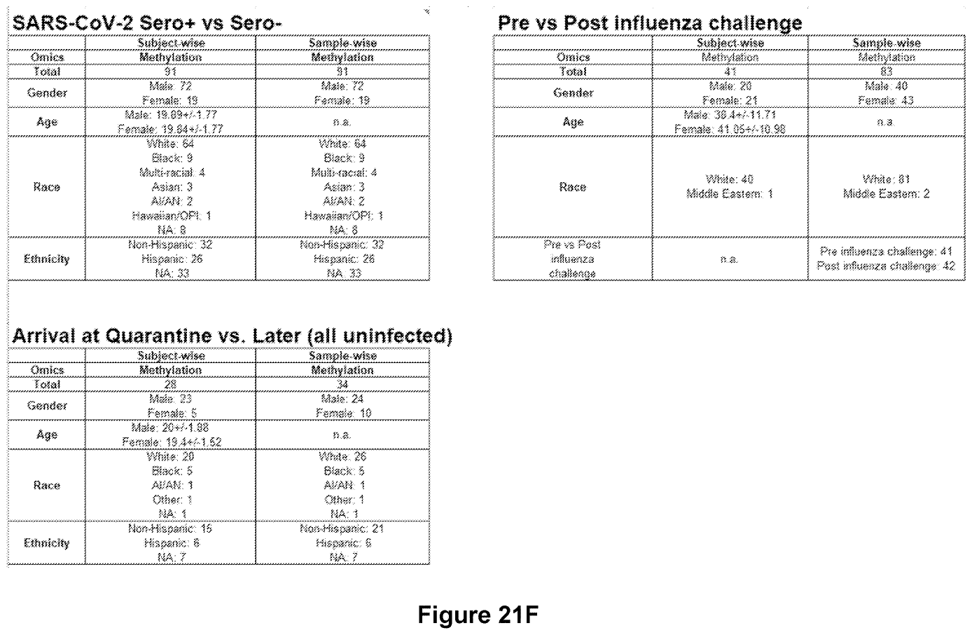

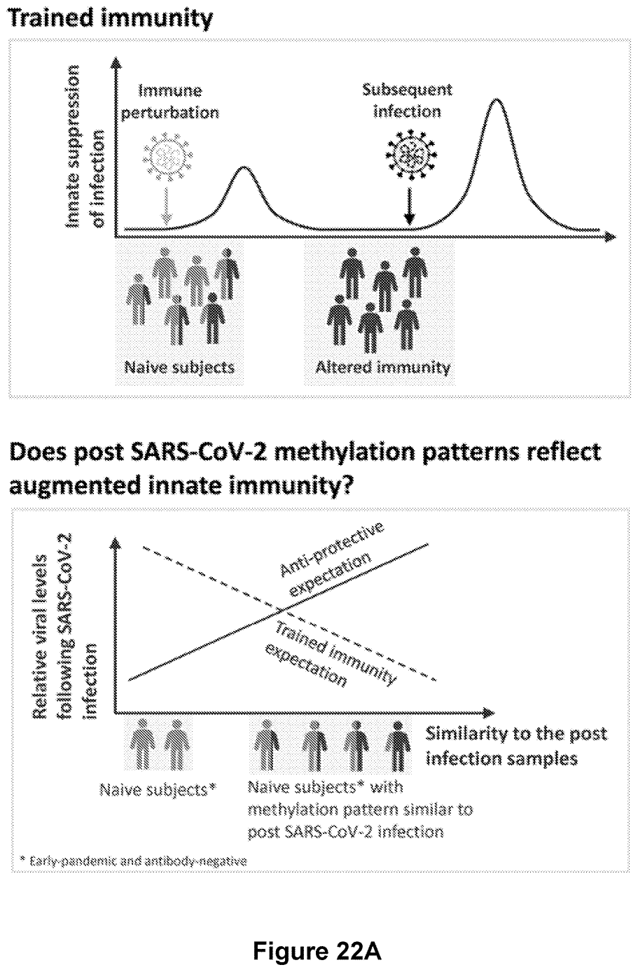

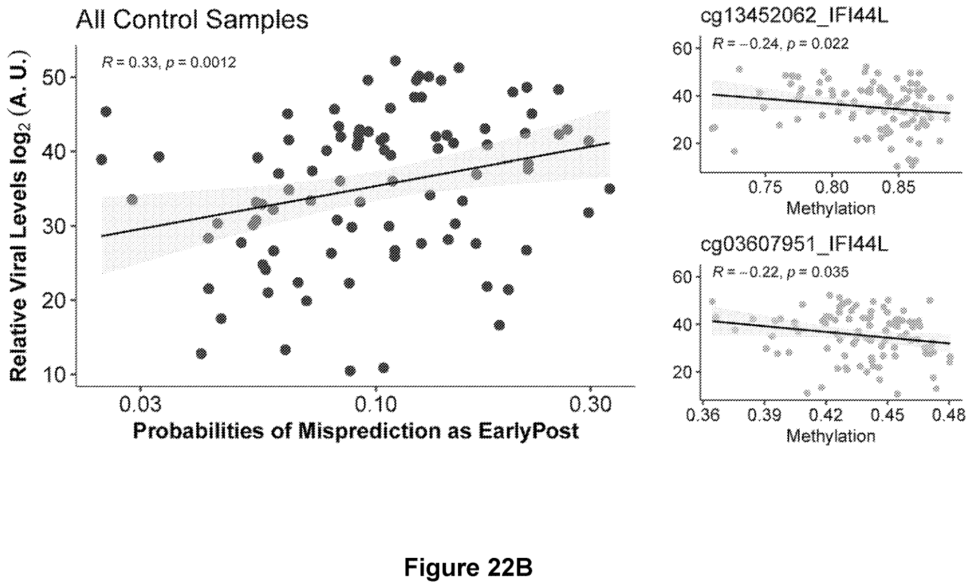

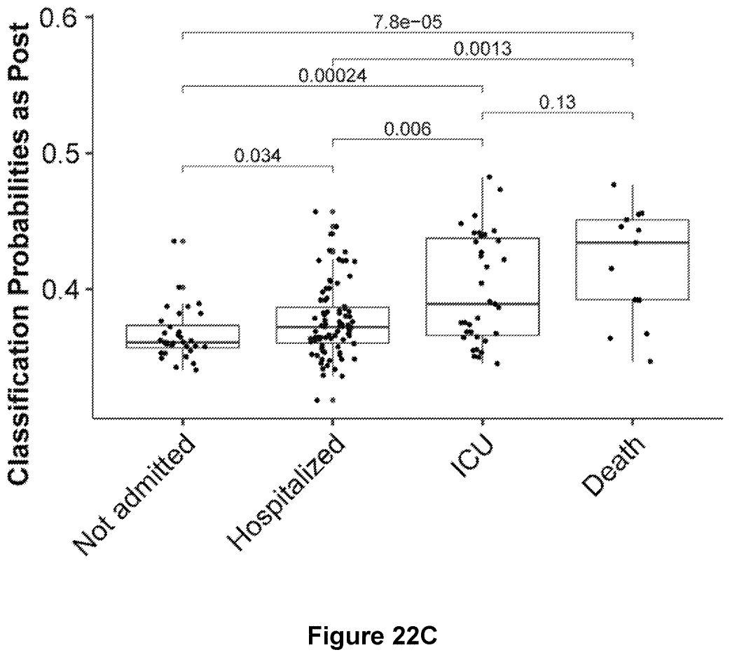

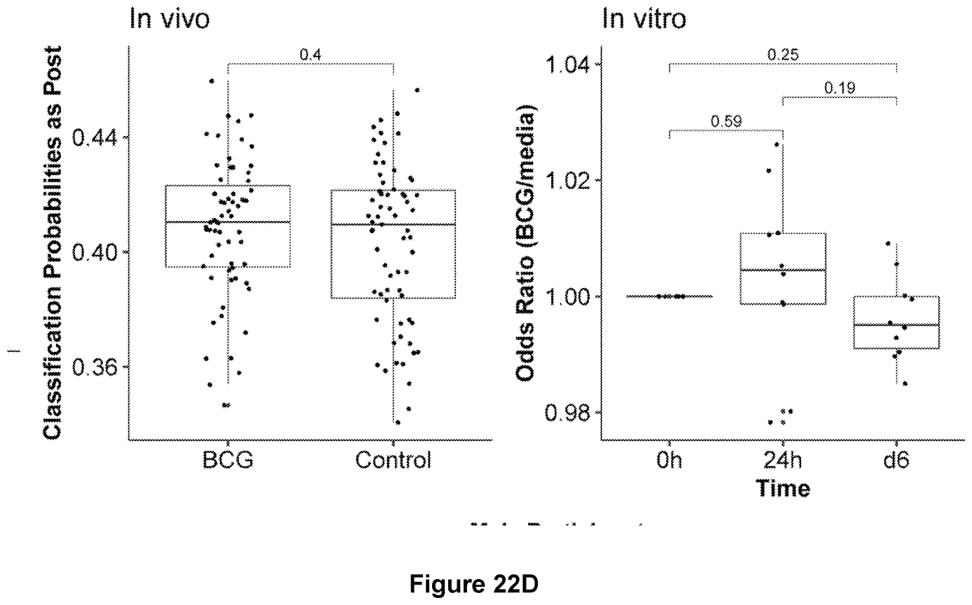

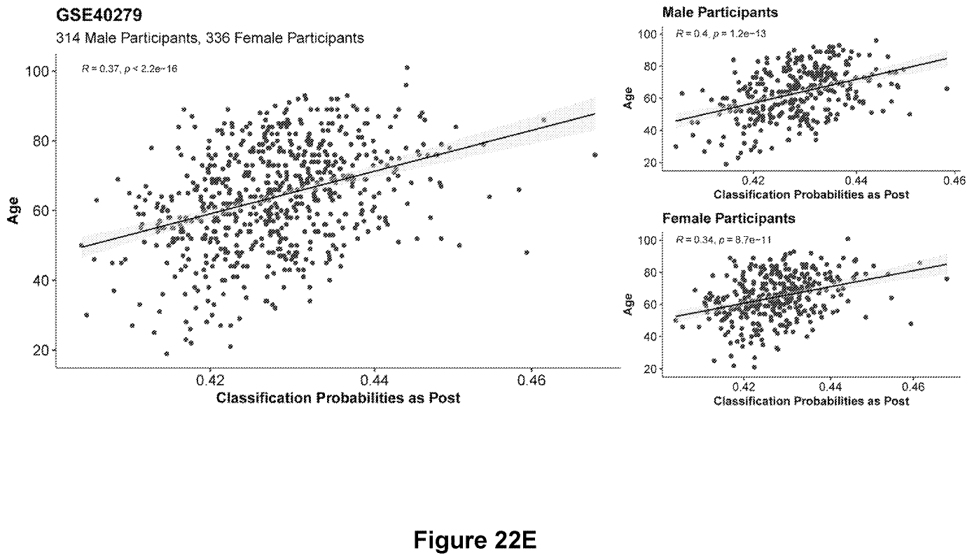

Epigenetic regulation following infection has in some instances been found to convey protection against subsequent infection challenge and this phenomenon is often referred to as trained immunity (Netea et al, 2020). On a mechanistic level trained immunity is attributed to a permissive epigenetic state that allows for faster upregulation of chemokines and receptors needed to mount an immune response. Trained immunity has been invoked to explain infection induced protection in animals that lack an adaptive immune system as well as cross-pathogen protection. The longitudinal nature of this cohort combined with a well-defined post-infection methylation state enabled the evaluation of whether the postinfection methylation state defined by this embodiment of the present disclosure is protective against infection (FIG. 22A).

It was reasoned that prior to infection, the methylation patterns in subsequently infected longitudinal study participants vary in their relative similarity to the methylation signatures post PCR positivity. In other words, the control samples could already be in a postinfection-like state, for example as a result of infection with a different infectious agent or another immune challenge such as vaccination. It is noted that the SARS-CoV-2 vaccine was not available at the time of this study. Thus, as a quantification of the similarity of preinfection control samples to the patterns seen following infection, the probability of these samples being misclassified to the active infection period (PCR+), the early period following infection (EarlyPost), or the later period following infection by the multiclass classifier (See FIG. EV5A of Mao et al., 2023, “A methylation clock model of mild SARS-CoV-2 infection provides insight into immune dysregulation,” Molecular Systems Biology 19 e:11361 which is hereby incorporated by reference) was used.

Whether similarity to the postinfection methylation state at baseline was predictive of the future response to SARSCoV-2 infection was examined. Because symptoms were so sparse in this cohort, the minimum SARS-CoV-2 PCR cycle (negated to indicate viral load in arbitrary units) was used as a measure of the effectiveness of controlling the virus infection. The relationship of the preinfection sample misclassification probabilities to the subsequent level of the virus was examined. Nearly all samples were, in fact, correctly classified by the model. The term “misclassification” here reflects merely the quantitative probability obtained from the model of classifying the samples as belonging to the wrong class. Probabilities of these samples being misclassified as active infection or LatePost were not significantly associated with viral load (See FIG. EV5B of Mao et al., 2023, “A methylation clock model of mild SARS-CoV-2 infection provides insight into immune dysregulation,” Molecular Systems Biology 19 e:11361 which is hereby incorporated by reference). The probabilities of the preinfection samples being misclassified as EarlyPost were associated with having higher maximal levels of virus detected by PCR (P=0.001, Spearman rank correlation; FIG. 22B). This result demonstrates that baseline methylation values were indeed predictive of future infection response. An identical analysis using gene expression did not yield significant results (See Appendix FIG. S3 of Mao et al., 2023, “A methylation clock model of mild SARS-CoV-2 infection provides insight into immune dysregulation,” Molecular Systems Biology 19 e:11361 which is hereby incorporated by reference), supporting a key role of the methylation-encoded epigenetic state.

However, while we demonstrate a clear predictive power for baseline methylation, the direction of association is the opposite to that found in trained immunity. If the postinfection-like state were protective, it would be expected to correlate with lower viral loads. Notably, it the opposite result were found (FIG. 22B). This result can be confirmed by looking at individual features that contribute to our Early-Post model. Among the top 16 CpG sites used by the model, two hypomethylated sites in IFI44L are highlighted, which were individually inversely correlated with virus level (FIG. 22B). These results suggest that individuals having preinfection blood methylation patterns similar to that characteristic of the post-PCR-positive period showed a less effective suppression of SARS-CoV-2 during infection.

In order to evaluate the generalizability of these findings to a more diverse cohort, we applied our postinfection model to a SARSCoV-2 infection dataset from a different cohort having a broader age range (50.6+17.2), more balanced sex composition (70 female, 92 male) and that included severe outcomes (Konigsberg et al, 2021). It was found that the postinfection probability calculated on methylation state early in the disease course was significantly associated with disease severity and death (FIG. 22C), further supporting the hypothesis that the state identified in the present disclosure is associated with reduced effectiveness of the immune response to SARS-CoV-2 infection.

The postinfection model was also applied to an in vivo and in vitro methylation study of BCG vaccination, one of the best-characterized perturbations for inducing trained immunity (Bannister et al, 2022). It was found that the similarity to the SARS-CoV-2 postinfection state was not significantly changed when comparing either the in vivo or the in vitro (FIG. 22D) pre- and post-BCG infection samples, further supporting the view that the epigenetic state identified in the present disclosure is distinct from trained immunity.

While the mechanistic details need to be further elucidated, the reasons for these contradictory findings can by contemplated. Both the gene expression and methylation data are heavily dominated by interferon-related genes and loci. Many interferon-induced genes (ISGs) have well-characterized antiviral activity and provide protection on the cellular and organismal levels (McNab et al, 2015). However, a growing body of evidence suggests that interferon signaling provides important immunoregulatory functions (Lee & Ashkar, 2018), and the effects of interferons on infection susceptibility are complex and context-dependent (McNab et al, 2015). Indeed, in the present disclosure, some of the most persistent hypomethylated loci are located near IFI44L and FKBP5, two genes that have been shown to negatively regulate antiviral responses (DeDiego et al, 2019a, 2019b). Together, these observations suggest that the epigenetic memory observed in the present disclosure may in fact reflect an interferon regulatory feedback state that correlates with reduced capacity for viral suppression. If this were the case, it is expected that the probability of being in a postinfection-like state as defined by the disclosed model should increase with the number of infections and thus with age. This conjecture is confirmed in several large cohorts of methylation data and find a similar relationship in both males and females (FIG. 22E). See also Appendix FIG. S4 of Mao et al., Id., which is hereby incorporated by reference. Overall, the disclosed results support the formulation that the baseline methylation state, but not gene expression, is predictive of response to subsequent infection challenge. However, the state identified following SARS-CoV-2 infection is antiprotective and represents an epigenetic phenomenon that is distinct from trained immunity.

### 2.4. Discussion

The present disclosure provides a fine grain characterization of the temporal dynamics of methylation changes following an acute perturbation. The disclosed results indicate that in immune-naive healthy young adults, asymptomatic and mild SARS-CoV-2 infections induced prolonged alterations of DNA methylation. The dynamics of these methylation changes observed during several months of follow up were used to develop a methylation clock that accurately predicts time since infection. These results suggest that in addition to the lifetime methylation clocks that have been described, the methylome also contains a record of the timing of environmental exposures.

These dynamic epigenetic processes may have important implications for health and disease. In the context of immunological stimuli, methylation and other induced epigenetic changes can provide faster induction of immune responses thus benefiting host defense. (Netea et al., 2020). The post-infection methylation signature the present disclosure defined is related to other pro-inflammatory conditions such as chronic infections and autoimmune diseases, with the association being particularly strong for Systemic Lupus Erythematosus (SLE). Strikingly, contrary to the trained immunity phenomenon, in this cohort the presence of an early post-infection-like methylation state prior to infection is anti-protective for the SARS-CoV-2 infection that occurred subset to these baseline measurements. This potentially deleterious effect of

SARS-CoV-2 infection may be relatively short-lived the presence of a late postinfection-like methylation state prior to infection found in the present disclosure showed only a nonsignificant trend towards being antiprotective. An increased subsequent infection risk has also been observed following other primary infections, such as measles (Behrens et al, 2020). The presence early after SARS-CoV-2 infection of a methylation state that is similar to the post-SARS-CoV-2 infection methylation state defined by the disclosed model is associated with poorer outcomes in a more diverse cohort. The state defined using the present disclosure is related to a regulatory feedback process that downregulates interferon activity and results in reduced viral suppression. Overall, the disclosed results suggest that the persistent SARS-CoV-2 methylation identified represents a dysregulated epigenetic state.

### 2.5. Materials and Methods

### Sources of Samples for Analysis

### COVID-19 Health Action Response for Marines Study (CHARM)

In some embodiments, the systems and methods of the present disclosure obtained samples as part of the prospective COVID-19 Health Action Response for Marines (CHARM) study, which followed predominantly male, US Marine recruits after a 2-week home quarantine. A second supervised 2-week quarantine followed, that included SARS-CoV-2 mitigation measures such as mask wearing and social distancing, along with daily temperature and symptom monitoring. At the time of arrival at quarantine, CHARM study participants were tested for SARS-CoV-2 infection via quantitative polymerase-chain-reaction (qPCR) assay of nasal swab specimen and evaluated for baseline SARS-CoV-2 IgG seropositivity, defined as a dilution of 1:150 or more on receptor-binding domain and full-length spike protein ELISA. SARS-CoV-2 infection and COVID-19-related symptoms or any other unspecified symptom were assessed at weeks 1 and 2 of quarantine. Study participants included Marines who had three negative PCR tests during quarantine and a baseline serum serology test that indicated them as either seropositive or seronegative for SARS-CoV-2. As recruits went on to basic training at Marine Corps Recruit Depot-Parris Island SC, PCR tests were performed at weeks 2, 4 and 6 in both seropositive and seronegative groups. Additionally, a baseline neutralizing antibody titer was measured on all subsequently seropositive participants, and a follow-up symptom questionnaire was provided. In some embodiments, the systems and methods of the present disclosure also collected PAXgene blood samples for RNA-seq analysis and EDTA blood samples for DNA methylation analysis from PBMCs. All samples were frozen at −80° C. after collection prior to processing for RNA-seq and methylation analysis. Additional details regarding CHARM study are described in (Letizia et al., 2021).

### Retrospective Study of US Marines

Marine recruits in training at Marine Corps Recruit Depot-Parris Island SC who were in companies exposed to SARS-CoV-2 during a cluster occurring from Mid-March to Mid-April 2020 were later enrolled in a retrospective blood sampling study. Only a few study participants had been tested for SARS-CoV-2 at the time of the cluster. Samples were obtained approximately 6 and 10 weeks after exposure, with the 10-week samples analyzed for the present study. EDTA blood samples were used for DNA methylation analysis from PBMCs. Additional details regarding this study and the serological analysis of these samples are described in Ramos et al. (2021). Notably, mild symptoms included runny nose, sore throat, cough, subjective fever, headache, chills, and nausea (see Table 1 in Ramos et al., 2021)).

### Influenza Challenge Study

Samples were analyzed from the placebo vaccination group from an influenza H3N2 (A/Belgium/2417/2015) virus human challenge model study. DNA methylation analysis was performed using cryopreserved PBMC collected from 41 participants before the challenge and 28 days after the challenge for each subject. Additional study details can be found at trial NCT03883113 at clinicaltrials.gov.

### Protection of Human Subjects

Institutional Review Board approval was obtained from the Naval Medical Research Center (protocol number NMRC.2020.0006) in compliance with all applicable US federal regulations governing the protection of human subjects. All participants provided written informed consent, and the experiments conformed to the principles set out in the WMA Declaration of Helsinki and the Department of Health and Human Services Belmont Report.

### Data Production

Total RNA Isolation and cDNA Library Preparation

RNA from PAXgene preserved blood was extracted using the Agencourt RNAdvance Blood Kit (Beckman Coulter, Indianapolis, IN) on a BioMek FXP Laboratory Automation Workstation (Beckman Coulter). Concentration and integrity (RIN) of isolated RNA were determined using the Quant-iT™ RiboGreen™ RNA Assay Kit (Thermo Fisher) and an RNA Standard Sensitivity Kit (DNF-471, Agilent Technologies, Santa Clara, CA, USA) on a Fragment Analyzer Automated CE system (Agilent Technologies), respectively. Subsequently, cDNA libraries were constructed from total RNA using the Universal Plus mRNA-Seq kit (Tecan Genomics, San Carlos, CA, United States) in a Biomek i7 Automated Workstation (Beckman Coulter). Briefly, mRNA was isolated from purified 300 ng total RNA using oligo-dT beads and used to synthesize cDNA following the manufacturer's instructions. The transcripts for ribosomal RNA (rRNA) and globin were further depleted using the AnyDeplete kit (Tecan Genomics) prior to the amplification of libraries. Library concentration was assessed fluorometrically using the Qubit dsDNA HS Kit (Thermo Fisher), and quality was assessed with the HS NGS Fragment Kit (1-6000 bp) (DNF-474, Agilent Technologies).

### RNA Sequencing and Preprocessing of the RNA-Seq Data

Following library preparation, samples were pooled and preliminary sequencing of cDNA libraries (average read depth of 90,000 reads) was performed using a MiSeq system (Illumina), to confirm library quality and concentration. Deep sequencing was subsequently performed using an S4 flow cell in a NovaSeq sequencing system (Illumina) (average read depth ~30 million pairs of 2×100 bp reads) at New York Genome Center.

### Methylation Data

All samples were frozen at −80° C. after collection prior to processing for methylation analyses. Genomic DNA was extracted from cryopreserved PBMC or blood collected in EDTA tubes using Genfind V3 (Beckman Coulter) on a BioMek FX Laboratory Automation Workstation (Beckman Coulter). All DNA samples were quantified using both absorbance (NanoDrop 2000; Thermo Fisher Scientific, Waltham, MA) and fluorescence-based methods (Qubit; Thermo Fisher Scientific, Waltham, MA) using standard dyes selective for double-stranded DNA, minimizing the effects of contaminants that affect the quantitation.

DNA methylation was quantified using Illumina Infinium Human Methylation EPIC Bead Chip array (Illumina Inc., San Diego, CA) according to the manufacturer's instructions at University of Minnesota Genomic Center. Briefly, 500 ng of DNA from each sample was treated with sodium bisulfite, using the EZ-96 DNA Methylation-Gold kit (Zymo Research, CA, USA). The bisulfite-converted amplified DNA products were denatured into single strands and hybridized to the Illumina Infinium Human Methylation EPIC Bead Chip array (Illumina Inc., San Diego, CA). The hybridized BeadChips were stained, washed, and scanned for the intensities of the un-methylated and methylated bead types using Illumina's iScan System. The DNA methylation beta values were obtained from the raw IDAT files by using the ChAMP package in R. Samples from the same individual were processed together across all experimental stages to negate any methodological batch effects.

### Data Processing and Quality Assessment

### RNA-seq

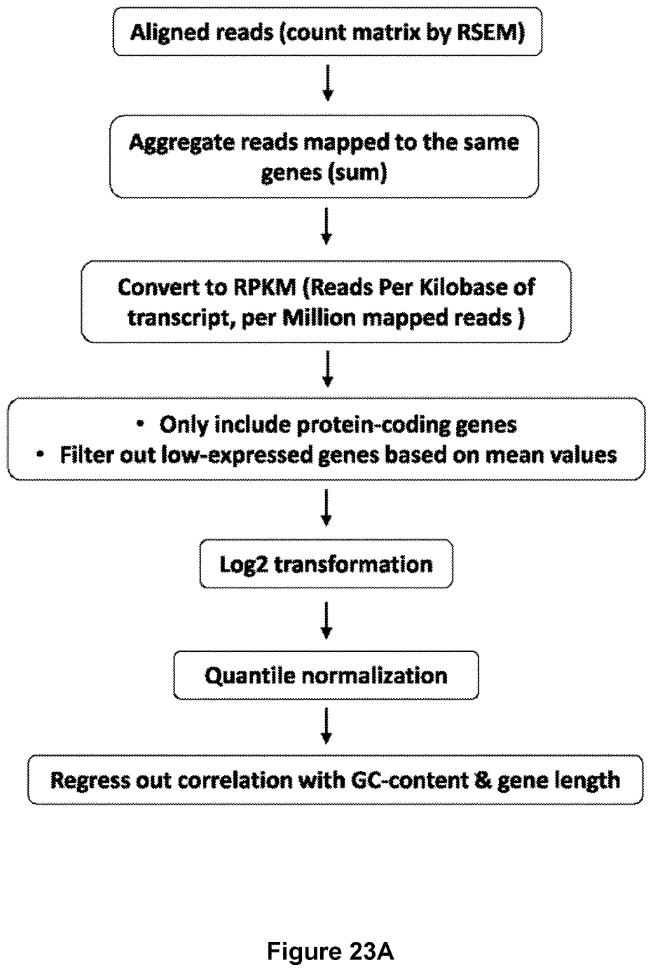

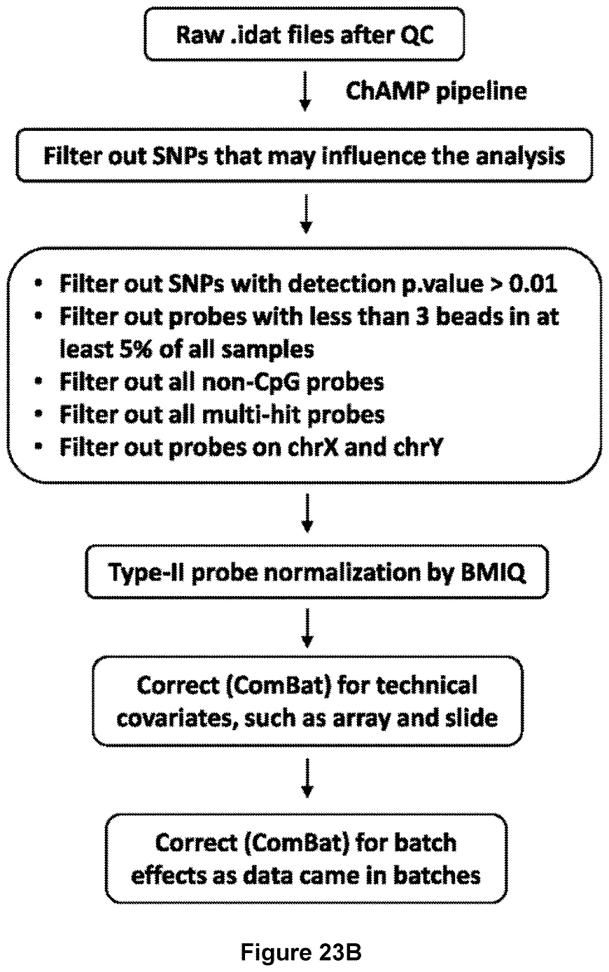

The RNA-seq reads were converted from raw RSEM counts to the final gene-level quantification following the pipeline in FIG. 23A. In some embodiments, the systems and methods of the present disclosure only included protein-coding genes and filtered out low-expressed genes based on the mean expression levels. Overall, the present disclosure had 11,436 genes left after filtering.

### Methylation

In some embodiments, the systems and methods of the present disclosure adopted the ChAMP pipeline (Tian et al, 2017) to process the raw (IDAT) files from Illumina Methylation microarray platform. The normalization steps and probe filtering criterion are illustrated in the FIG. 23B. In some embodiments, the systems and methods of the present disclosure applied ComBat (Johnson and Rabinovic, 2007) in the M-value space to regress out potential technical covariates including Array (EPIC array), Slide (EPIC array) and batches (EPIC array plates). Then the present disclosure converted methylation levels of 707,361 CpG sites from M-values to beta-values for all the downstream analysis.

For both RNA-seq and methylation samples, only samples from subjects who were PCR- and serology negative when enrolled in the study were kept for the downstream analysis. In some embodiments, the systems and methods of the present disclosure further filtered out samples if they were outliers in the principal component (PC) space. In some embodiments, the systems and methods of the present disclosure calculated the Mahalanobis distances to the center in the PC space of the first 5 principal components correspondingly. As the distances follow a chi-square distribution, samples with significant p-values (0.01 divided by number of samples included in the test) were classified as outliers. In total, there were 2 methylation samples, and 3 RNA-seq samples excluded from downstream analysis.

### Computational Inference of Cell Type Proportions

### RNA-Seq

In some embodiments, the systems and methods of the present disclosure only used genes included in Cibersort LM22 (Newman et al, 2015) to estimate the proportions of six major cell types. In some embodiments, the systems and methods of the present disclosure first trained an elastic net model (Friedman et al, 2010) (alpha=0.9, 10-fold CV) to predict the inferred cell type proportions based on paired methylation data. Then the present disclosure selected lambda corresponding to the minimum cross-validation error to generate predictions for the complete RNA-seq data. Similarly, the present disclosure regressed out inferred cell type proportions by linear regression from the uncorrected gene expression profiles. The gene expression profiles that were corrected for cell type proportions would be used for some downstream analysis.

### Methylation

The ChAMP pipeline (Tian et al, 2017) was adopted to process the raw (IDAT) files from Illumina Methylation microarray platform. The normalization steps and probe filtering criterion are illustrated in FIG. 23B. ComBat (Johnson et al, 2007) was applied in the Mvalue space to regress out potential technical covariates including Array (EPIC array), Slide (EPIC array), and batches (EPIC array plates). Then, methylation levels of 707,361 CpG sites were converted from M-values to beta values for all downstream differential methylation analysis and modeling. The regression of cell-type proportion to remove the confounding effect used for clustering was performed in both beta value and M-value space, with the results obtained in M-value space (see Materials and Methods, Subsection Temporal clustering).

For both RNA-seq and methylation samples, only samples from subjects who were PCR- and serology-negative when enrolled in the study were kept for the downstream analysis (FIG. EV1). Samples were further filtered out if they were outliers in the principal component (PC) space. Mahalanobis distances were calculated to the center in the PC space of the first five principal components correspondingly. As the distances follow a chi-square distribution, samples with significant P-values (0.01 divided by the number of samples included in the test) were classified as outliers. In total, there were two methylation samples, and three RNA-seq samples excluded from downstream analysis.

### Computational Inference of Cell-Type Proportions

### Methylation

Proportions of six major cell types (B cells, Granulocytes, Monocytes, NK cells, CD4 T cells, and CD8 T cells) were estimated using a standard reference-based method (Houseman et al, 2012). The original CellType450K basis matrix was takend and replaced the values with those from (Roy et al, 2021; Illumina Methylation microarray). This was done to help remove bias induced by the platform inconsistency. Cell-type specificity obtained with the updated basis matrix was compared to that obtained using the standard Houseman et al (2012) basis. It was found that the cell-type specificity blocks were preserved and in some cases actually improved in the updated matrix. In particular, it was found that the hypomethylated values are generally lower in the new basis (Appendix FIG. S9A and B of Mao et al., 2023, “A methylation clock model of mild SARS-CoV-2 infection provides insight into immune dysregulation,” Molecular Systems Biology 19 e:11361 which is hereby incorporated by reference). The overall correlation of the standard basis values against the updated basis values is nearly perfect (Appendix FIG. S9C of Mao et al., 2023, “A methylation clock model of mild SARS-CoV-2 infection provides insight into immune dysregulation,” Molecular Systems Biology 19 e:11361 which is hereby incorporated by reference). The differential methylation site analysis was performed on raw beta values using these cell-type proportions as covariates (see Materials and Methods, Sub-section Differential gene and methylation site analysis). For clustering analysis, a cell-typecorrected matrix was created by regressing out cell-type proportions first (see our elaboration in Sub-section Temporal clustering). The machine learning models used the raw beta value matrix (see Subsection Machine learning models).

### RNA-Seq

A goal for proportion inference was to ascertain whether the major trends in our data such as more prolonged alterations in DNA versus RNA were insensitive to cell proportion correction. As proportion estimation from RNA and methylation differs greatly in terms of robustness and the number of cell types that can be estimated (methylation is more robust while RNA can be used to estimate some rare cell types) in order to formulate a fair comparison both modalities were corrected for the same cell proportion estimates.

The methylation estimated proportions were used as a gold standard. For RNA samples with no matching methylation, the proportions were imputed using a simple machine learning model. Genes included in Cibersort LM22 (Newman et al, 2015) were used to train an elastic net model (Friedman et al, 2010; a=0.9, 10-fold CV) to predict the inferred cell-type proportions based on paired methylation data. Then, lambda corresponding to the minimum cross-validation error were selected to generate predictions for the complete RNAseq data. Similarly, inferred cell-type proportions were regressed out by linear regression from the uncorrected gene expression profiles. The gene expression profiles that were corrected for cell-type proportions were used for some downstream analysis. It was found that using alternative methods of proportion estimation including a newly published methylation basis with 12 cell types (Salas et al, 2022) and CIBERSORTx (Newman et al, 2019) did not alter the main conclusions. Alternative versions were produced, which shows the timing of methylation and RNA changes, using different proportion estimation methods and find that the overall trend is unchanged (Appendix FIG. S5 of Mao et al., 2023, “A methylation clock model of mild SARS-CoV-2 infection provides insight into immune dysregulation,” Molecular Systems Biology 19 e:11361 which is hereby incorporated by reference). Cell proportion differences were also visualized across time points in Appendix FIG. S6 of Mao et al., 2023, “A methylation clock model of mild SARS-CoV-2 infection provides insight into immune dysregulation,” Molecular Systems Biology 19 e:11361 which is hereby incorporated by reference.

### Differential Gene and Methylation Site Analysis

In some embodiments, the systems and methods of the present disclosure adopted limma (Ritchie et al, 2015) to perform differential analysis for both methylation data and RNA-seq data. In some embodiments, the systems and methods of the present disclosure noted that many methylation probes with similar time trajectory patterns had highly variable value ranges. To account for this, the present disclosure transformed the beta values into z-scores. Subsequent methylation analysis was performed using limma in this standardized space. Because the standardization is a linear transformation, it does not affect the significance of the limma linear model coefficients. The differential output from the limma analysis is referred to as log fold change for the RNA data and as normalized delta-beta for the methylation data. The present disclosure included age and sex as biological covariates in the limma models when cell type proportions were not corrected. When cell type proportions were corrected, the proportions of six major cell types (Monocyte %, Bcell %, Gran %, CD4T %, CD8T %, NK %) were also included as biological covariates. The raw P-values were corrected by Benjamini-Hochberg (BH) method and significance cutoff of FDR<0.05 was applied.

Comparison of Methylation after Symptomatic and Asymptomatic Infections

The participant symptom category (symptomatic, asymptomatic) was determined by the result of temperature screening and a 14-symptom questionnaire obtained concerning the week prior to each study visit. For details, see Letizia et al (2021). Responses covering up to 2 weeks before and after the initial PCR-positive test were used for group assignment. Differential analysis comparing these symptomatic and asymptomatic participants separately for each time period (Control, First, Mid, EarlyPost, and LatePost; see Table 2.5 and 2.6) was performed.

### Temporal Clustering

The present disclosure clustered CpG sites that were aligned to the first PCR positive day for each subject. In some embodiments, the systems and methods of the present disclosure only included time points with more than four associated samples, giving 20 time points. The beta value matrix was corrected for cell type proportions. In some embodiments, the systems and methods of the present disclosure first fitted a loess (local polynomial regression fitting) curve for each CpG site, then the present disclosure discretized the fitted curve and only kept the values corresponding to the 20 unique time points.

CpG sites were clustered with respect to these discrete time series, and the similarity of each pair of time series was evaluated using dynamic time-warping distance (Leodolter et al, 2021). Dynamic time-warping is an algorithm that calculates the optimal matching between two time series (Liu & Muller, 2003; Leng & Muller, 2006). It measures similarity based on overall trajectory, regardless of speed. These characteristics make it beneficial for clustering differential features according to their temporal trajectory patterns. The warping window size was set to be 20. The distance matrix was squared and then used as input for the hierarchical clustering step (Ward's minimum variance method, seven clusters). In summary, the temporal clustering analysis includes four consecutive steps: (i) correct for cell-type proportions, (ii) smooth the normalized data by local polynomial regression fitting, (iii) calculate the dynamic timewarping distance matrix, and (iv) run hierarchical clustering using the distance matrix as input. Two different approaches to correct for the celltype proportions were investigate: the first approach named B2M2B is to first convert the beta value matrix to M-value matrix, regress out cell-type proportions in the M-value space by linear regression, and convert the M-value matrix back to the beta value space. An alternative approach was considered where cell-type proportions were directly regressed out in the beta value space, and this approach is termed herein B_regress (see Appendix FIG. S7A of Mao et al., 2023, “A methylation clock model of mild SARS-CoV-2 infection provides insight into immune dysregulation,” Molecular Systems Biology 19 e:11361 which is hereby incorporated by reference). Selection between these two normalization strategies (B2M2B vs. B_regress) was done by running through the same pipeline detailed above with all hyperparameters fixed in steps (2-4) and comparing all the intermediate outputs side by side. First, B2M2B and B_regression generated nearly identical beta value matrices after correcting for cell-type proportions (see Appendix FIG. S7B of Mao et al., 2023, “A methylation clock model of mild SARS-CoV-2 infection provides insight into immune dysregulation,” Molecular Systems Biology 19 e:11361 which is hereby incorporated by reference). Next, the corresponding dynamic warping distance matrices were also highly correlated (see Appendix FIG. S7C of Mao et al., 2023, “A methylation clock model of mild SARS-CoV-2 infection provides insight into immune dysregulation,” Molecular Systems Biology 19 e:11361 which is hereby incorporated by reference). Finally, the cluster assignments were compared after running through the hierarchical clustering step. Due to the NP-hard nature of the hierarchical clustering problem, Ward's minimum variance method tried to minimize the total within-cluster variances (SSE) in a heuristic manner in practice, and the different initializations might end up with different local optimal solutions. B_regress resulted in a larger total within-cluster variance (SSE) (see Appendix FIG. S7A of Mao et al., 2023, “A methylation clock model of mild SARS-CoV-2 infection provides insight into immune dysregulation,” Molecular Systems Biology 19 e:11361 which is hereby incorporated by reference), indicating that the corresponding cluster assignment was indeed less tight compared with that based on B2M2B. From the perspective of the clustering optimization problem, the B2M2B cluster assignment was a better solution. The biological coherence of the resulting clusters was also investigated using the downstream enrichment pipeline (see Materials and Methods, Sub-section Enrichment analysis by temporal cluster). It was found that the B2M2B cluster assignment was also more biologically coherent, as the corresponding transcription factor (TF) enrichment results identified unique enriched TFs for all seven clusters, whereas the B_regress analysis failed to identify unique TFs that were significantly enriched with Cluster 2, 6 and 7 (see FIG. 2 and Appendix FIG. S8 of Mao et al., 2023, “A methylation clock model of mild SARS-CoV-2 infection provides insight into immune dysregulation,” Molecular Systems Biology 19 e:11361 which is hereby incorporated by reference). The clustering analysis and annotations based on the B2M2B method in FIG. 2 of of Mao et al., 2023, “A methylation clock model of mild SARS-CoV-2 infection provides insight into immune dysregulation,” Molecular Systems Biology 19 e:11361 and the parallel analysis using B_regress is shown in Appendix FIG. S8 of Mao et al., 2023, “A methylation clock model of mild SARS-CoV-2 infection provides insight into immune dysregulation,” Molecular Systems Biology 19 e:11361 which is hereby incorporated by reference.

### Enrichment Analysis by Temporal Cluster

These enrichment analyses comparing each cluster with the other clusters with respect to both discrete phenotypes, continuous phenotypes and transcription factor binding sites are presented in FIGS. 19A-19C. See also FIG. EV3 of Mao et al., 2023, “A methylation clock model of mild SARS-CoV-2 infection provides insight into immune dysregulation,” Molecular Systems Biology 19 e:11361 which is hereby incorporated by reference.

### Pathway/Cell Markers and Discrete Phenotype Enrichment Analysis

In some embodiments, the systems and methods of the present disclosure first mapped DMS to associated genes based on Illumina Methylation microarray annotation. If multiple DMS were mapped to the same gene, the corresponding gene would be only included as foreground or background once. In some embodiments, the systems and methods of the present disclosure combined canonical pathways and hallmark pathways from MsigDB (v7.4) (Liberzon et al., 2015; Liberzon et al., 2011) together to formulate a comprehensive pathway set. The other discrete phenotypes included cell markers (scRNA-seq) (Stuart et al., 2019), gene region feature categories and CpG island categories. In some embodiments, the systems and methods of the present disclosure adopted the hypergeometric test by cluster to conduct enrichment analysis.

### Continuous Phenotype Enrichment Analysis

For each DMS, the present disclosure collected four different categories of continuous phenotypes. The first category was the Blueprint Epigenome project cell type signatures (Stunnenberg, 2016). In some embodiments, the systems and methods of the present disclosure downloaded the bigWig file matching “CPG_methylation_calls.bs_call.GRCh38” from Blueprint. Beta values corresponding to EPIC array probes were extracted using bwtool (Pohl & Beato, 2014). Missing values were imputed using knn.impute and the replicates were mean summarized. CpG levels were z-scored to define relative cell-type specificity. In some embodiments, the systems and methods of the present disclosure calculated the spearman rank correlations between one hot encoding of the cluster membership of all DMS and the corresponding normalized Blueprint CpG levels to test for significant associations. The second category was the correlation with ref-based cell type proportions. This was defined as the Pearson correlations of DMS methylation levels and the inferred proportions of six major cell types (B cells, Granulocytes, Monocytes, NK cells, CD4 T cells and CD8 T cells). The third class was the CG pattern/GC pattern/GC ratio. The CG pattern was defined as the number of CpG (dinucleotides) divided by N−1 (number of dinucleotide positions), and the GC pattern was defined as the number of GpC divided by the number of dinucleotide positions. GC ratio was the ratio of G/C mono-nucleotides. The last class was the distance of each DMS to the closest transcription start sites (TSS). In some embodiments, the systems and methods of the present disclosure ranked DMS based on each class of the continuous phenotypes and conducted the Wilcoxon rank sum test for enrichment analysis.

### Transcription Factor Enrichment Analysis

Homer (v4.11; Heinz et al, 2010) was utilized to test the enrichment of transcription factor binding sites by cluster within a 200 bp window centered at each DMS. The transcription factors included in the analysis were the 440 known motifs for vertebrates included in Homer. When the 200 bp windows of one cluster were specified as the foreground sequences, the 200 bp windows of other clusters were used as the background.

### Enrichment Analysis of Reported Differential CpG Sites

In FIG. 21D and FIG. 21E, the present disclosure tested whether reported differentially methylated CpG sites of other diseases were enriched with respect to the rankings in the longitudinal study. For many published studies, the present disclosure found that de novo analysis of the raw data did not replicate the DMS rank lists reported by the authors. In some embodiments, the systems and methods of the present disclosure reasoned that the discrepancies most likely resulted from the selection of covariates, and because the original authors had privileged knowledge about covariates that may improve the analysis, the present disclosure used the published DMS calls from each study for our comparative analysis. Accordingly, the present disclosure extracted the DMS from each published manuscript and ordered them based on the absolute delta beta values. Then the present disclosure took the top 20 hypomethylated sites and tested whether they were enriched given the rankings (ordered by absolute delta beta values) of significantly hypomethylated sites (EarlyPost vs Control or LatePost vs Control) from the analysis of the longitudinal CHARM study data using Wilcoxon rank sum test.

### Machine Learning Models

### Overview of Model Construction

In some embodiments, the systems and methods of the present disclosure utilized a nested cross validation strategy to build different prediction models for the longitudinal study. There are two loops in the nested cross validation procedure where an “inner” cross-validation step is nested inside an “outer” train-test split. The nested cross validation strategy eliminates the possibility of selection bias when constructing the test-train split and more accurately estimates the generalization error of the model.

Unless otherwise specified, there were 100 outer train-test splits. In some embodiments, the systems and methods of the present disclosure used the elastic net model for both regression and classification tasks as the inner cross validation model. The input was the raw beta value matrix or gene expression profile without correcting for the cell type proportions. The average predictions reported in the manuscript (FIGS. 20A-20D, FIGS. 21A-22C) were calculated in two steps. See also FIGS. EV5B and Appendix FIG. S3 of Mao et al., 2023, “A methylation clock model of mild SARS-CoV-2 infection provides insight into immune dysregulation,” Molecular Systems Biology 19 e:11361 which is hereby incorporated by reference. First the test predictions (classification probabilities or values of response variables) were averaged for each sample using outer train-test splits that include this sample in the test set. Then the present disclosure took the average predictions of all samples to evaluate the AUC (classification) or the correlation value (regression) with respect to the ground truth. These metrics were referred to as the average AUC and the average correlation. In order to build an applicable model for the external dataset, the present disclosure first selected features that were robust (frequently selected over all outer train-test splits) and then built the model only with these most stable features.

### Binary Classification (FIGS. 20C, FIGS. 21B-1C)

In some embodiments, the systems and methods of the present disclosure constructed a binary classification model for each pair out of four defined groups: Control, PCR+ (combining First and Mid together), EarlyPost and LatePost (FIG. 20C). All 707,361 CpGs were included as features without pre-selection. 10% of the available data were used as the test set for each outer train-test split, and the present disclosure utilized the elastic net model (glmnet(family=“binomial”)) for the inner cross-validation step (alpha=0.9, 5-fold cross validation).

In some embodiments, the systems and methods of the present disclosure also built a binary classification model distinguishing Control samples with Post samples (including both EarlyPost and LatePost samples). All 707,361 CpGs were included as features without pre-selection. After the nested cross validation step, the present disclosure selected features that were most frequently utilized across outer iterations (>90% of all outer train-test splits, shown in dataset EV 11 of Mao et al., 2023, “A methylation clock model of mild SARS-CoV-2 infection provides insight into immune dysregulation,” Molecular Systems Biology 19 e:11361 which is hereby incorporated by reference) to build the model for unseen data.

Features were transformed into z-scores to build an elastic net model (alpha=0.9, 5-fold cross validation). Features were also first standardized before applying this pre-trained model on other datasets (FIG. 21B and FIG. 21C). If the dataset was based on the HM450K microarray, the present disclosure imputed the CpG sites that are not available on the HM450K microarray by all-zero vectors. In some embodiments, the systems and methods of the present disclosure utilized Wilcoxon rank sum test to estimate the significance of AUCs and and the adjusted P-values were calculated following the Benjamini-Hochberg correction.

### Multiclass Classification (FIG. 20C and FIG. 21A)

In some embodiments, the systems and methods of the present disclosure built a multi-class classification model with 10% of the available data as the test set for each outer train-test split. All 707,361 CpGs were included as features without pre-selection, and the present disclosure utilized the elastic net model (glmnet(family=“multinomial”)) for inner cross-validation step (alpha=0.9, 5-fold cross validation).

### Regression (FIG. 20A)

In some embodiments, the systems and methods of the present disclosure built a regression model using 10% of the available data as the test set for each outer train-test split. All 707,361 CpGs were included as features without pre-selection, and the present disclosure utilized the elastic net model (glmnet(family=“gaussian”)) for inner cross-validation step (alpha=0.5, 5-fold cross validation). In some embodiments, the systems and methods of the present disclosure repeatedly constructs the regression model for each time window following the same steps above.

### Methylation-Gene Annotation

The CpG-gene assignment is based on Illumina Methylation microarray annotation (manufacturer's manifest) for Genome assembly GRCh37 (hg19). The manifest also includes information on gene region feature categories and CpG island annotations. In this analysis the present disclosure categorized gene region feature categories into two main groups: promoter sites (including TSS1500, TSS200, 1st Exon and 5′ UTR) and gene body sites (including 3′ UTR, Body and ExonBnd annotations). The definition of these gene region feature categories can be found in (Illumina, 2014).

### Data Availability

All data needed to evaluate the conclusions in part 2 are present in the paper and/or the Supporting Information of Mao et al., 2023, “A methylation clock model of mild SARS-CoV-2 infection provides insight into immune dysregulation,” Molecular Systems Biology 19 e:11361 which is hereby incorporated by reference. The datasets produced in this disclosure (part 2) are available in the following databases.

RNA-seq data: Gene Expression Omnibus GSE198449 (https://www.ncbi.nlm.nih.gov/geo/query/acc.cgi?acc=GSE198449)

Methylation data: Gene Expression Omnibus GSE219037 (https://www.ncbi.nlm.nih.gov/geo/query/acc.cgi?acc=GSE219037)

### Part 3: Systems and Methods for Benchmarking Transcriptional Host Response Signatures for Infection Diagnosis

### Description:

The present disclosure provides a novel framework for systematic quantification of the robustness and cross-reactivity of a candidate signature based on curation and integration of a massive public data compendium and development of a standardized signature scoring method. In some embodiments, the disclosure provides an inherent trade-off between robustness and cross-reactivity.

Provided are systems and methods for providing a general evaluation framework for systematic quantification of robustness and cross-reactivity of a candidate signature, based on: (1) curation of massive public data and (2) development of a standardized signature scoring method. In some embodiments, the data compendium and evaluation framework developed herein provide a foundation for the development of signatures for clinical application.

One aspect of the present disclosure in accordance with Part 3 provides a method of evaluating a gene signature associated with a target condition that can afflict a host species, wherein the gene signature comprises a first plurality of positive genes that are up-regulated when the test subject has the target condition and a second plurality of genes that are down-regulated when the test subject has the target condition. The method comprises obtaining an indication of each gene in the first plurality of positive genes. The method further comprises obtaining an indication of each gene in the second plurality of negative genes. The method further comprises obtaining a plurality of datasets, where each dataset in the plurality of datasets includes transcriptional data for each respective subject in a corresponding plurality of subjects and an indication of whether the respective subject has or does not have a respective test condition in a plurality of test conditions. The plurality of datasets includes at least one dataset for each test condition in the plurality of test conditions. At least one test condition in the plurality of test conditions is the target condition.

In the method, for each respective dataset in a plurality of datasets, for each respective time point in a set of time points represented by the respective dataset: for each respective subject in the respective dataset, determining a score for the respective subject at the respective time point by determining a difference between a geometric mean of abundance values for the first plurality of positive genes and a geometric mean of abundance values for the second plurality of positive genes indicated in the respective dataset, an area under a receiver operator characteristic curve (AUROC) value is determined for the respective dataset for the test condition using the respective score for each subject in the respective dataset at each respective timepoint.

The method further comprises evaluating a performance of the gene signature using the AUROC value of each dataset in the plurality of datasets associated with the target condition; The method further comprises evaluating a cross-reactivity of the gene signature from the AUROC value of each dataset in the plurality of datasets associated with a test condition that is other than the target condition.

In some embodiments, the plurality of datasets comprises 10 or more datasets, 100 or more datasets, 1000 or more datasets, or 10,000 or more datasets.

In some embodiments, the target condition is an infection from a predetermined virus species.

In some embodiments, the target condition is an infection from a predetermined bacterial species.

In some embodiments, the plurality of test conditions represents viral infections from 10 or more different viral species, 20 or more different viral species, or 30 or more viral species.

In some embodiments, the plurality of test conditions represents bacterial infections from 10 or more different bacterial species, 20 or more different bacterial species, or 30 or more different bacterial species.

In some embodiments, the set of time points consists of a single time point and the cross-reactivity of the gene signature is a mean of the AUROC value of each dataset in the plurality of datasets associated with a test condition that is other than the target condition.

In some embodiments, the set of time points is a plurality of time points, the maximal AUROC value for each dataset in the plurality of datasets associated with the target condition is used to determine the performance of the gene signature, and the maximal AUROC value for each dataset in the plurality of datasets associated with a test condition that is other than the target condition is used to determine the cross-reactivity of the gene signature.

In some embodiments, each respective dataset in the plurality of datasets has, for each respective subject in the respective dataset, RNA-seq data for each gene in the first plurality of positive genes and each gene in the second plurality of positive genes, and each dataset in the plurality of datasets comprises twenty or more subjects.

In some embodiments, the target condition is a first cancer type and each test condition in the plurality of test conditions is a different second cancer type.

In some embodiments, target condition is a first degree of severity of a viral infection in the host species and a test condition in the plurality of test conditions is a second degree of severity of a viral infection in the host species.

In some embodiments, the host species is human.

In some embodiments, the first plurality of positive genes consists of between three and thirty genes of the host species, and the second plurality of negative genes consists of between three and thirty genes of the host species, other than the first plurality of positive genes.

In some embodiments, the first plurality of positive genes consists of between three and one hundred genes of the host species, and the second plurality of negative genes consists of between three and one hundred genes of the host species, other than the first plurality of positive genes.

In some embodiments, each dataset in the plurality of datasets comprises thirty or more subjects, forty or more subjects, 100 or more subjects, or between 5 and 1000 subjects.

Another aspect in accordance with part 3 of the present disclosure provides a computer system for evaluating a gene signature associated with a target condition that can afflict a host species, where the gene signature comprises a first plurality of positive genes that are up-regulated when the test subject has the target condition and a second plurality of genes that are down-regulated when the test subject has the target condition. The computer system comprises one or more processors and memory addressable by the one or more processors. The memory stores at least one program for execution by the one or more processors. The at least one program comprises instructions for obtaining an indication of each gene in the first plurality of positive genes. The at least one program further comprises instructions for obtaining an indication of each gene in the second plurality of negative genes. The at least one program further comprises instructions for obtaining a plurality of datasets. Each dataset in the plurality of datasets includes transcriptional data for each respective subject in a corresponding plurality of subjects and an indication of whether the respective subject has or does not have a respective test condition in a plurality of test conditions. The plurality of datasets includes at least one dataset for each condition in the plurality of test conditions. At least one test condition in the plurality of test conditions is the target condition.

The at least one program further comprises instruction for each respective dataset in a plurality of datasets, for each respective time point in a set of time points represented by the respective dataset: for each respective subject in the respective dataset, determining a score for the respective subject at the respective time point by determining a difference between a geometric mean of abundance values for the first plurality of positive genes and a geometric mean of abundance values for the second plurality of positive genes indicated in the respective dataset, determining an area under a receiver operator characteristic curve (AUROC) value for the respective dataset for the test condition using the respective score for each subject in the respective dataset at each respective timepoint.

The at least one program further comprises instructions for evaluating a performance of the gene signature using the AUROC value of each dataset in the plurality of datasets associated with the target condition.

The at least one program further comprises instructions for evaluating a cross-reactivity of the gene signature from the AUROC value of each dataset in the plurality of datasets associated with a test condition that is other than the target condition.

Another aspect in accordance with part 3 of the present disclosure provides a non-transitory computer readable storage medium. The non-transitory computer readable storage medium stores instructions, which when executed by a computer system, cause the computer system to perform a method for evaluating a gene signature associated with a target condition that can afflict a host species, where the gene signature comprises a first plurality of positive genes that are up-regulated when the test subject has the target condition and a second plurality of genes that are down-regulated when the test subject has the target condition.

The method comprises obtaining an indication of each gene in the first plurality of positive genes. The method further comprises obtaining an indication of each gene in the second plurality of negative genes. The method further comprises obtaining a plurality of datasets. Each dataset in the plurality of datasets includes transcriptional data for each respective subject in a corresponding plurality of subjects and an indication of whether the respective subject has or does not have a respective test condition in a plurality of test conditions. The plurality of datasets includes at least one dataset for each condition in the plurality of test conditions. At least one test condition in the plurality of test conditions is the target condition.

The method further comprises, for each respective dataset in a plurality of datasets, for each respective time point in a set of time points represented by the respective dataset: for each respective subject in the respective dataset, determining a score for the respective subject at the respective time point by determining a difference between a geometric mean of abundance values for the first plurality of positive genes and a geometric mean of abundance values for the second plurality of positive genes indicated in the respective dataset. The method further comprises determining an area under a receiver operator characteristic curve (AUROC) value for the respective dataset for the test condition using the respective score for each subject in the respective dataset at each respective timepoint.

The method further comprises evaluating a performance of the gene signature using the AUROC value of each dataset in the plurality of datasets associated with the target condition; The method further comprises evaluating a cross-reactivity of the gene signature from the AUROC value of each dataset in the plurality of datasets associated with a test condition that is other than the target condition.

### 3.1. Abstract

Identification of host transcriptional response signatures has emerged as a new paradigm for infection diagnosis. For clinical applications, signatures must robustly detect the pathogen of interest without cross-reacting with unintended conditions. To evaluate the performance of infectious disease signatures, the present disclosure developed a framework that includes a compendium of 17,105 transcriptional profiles capturing infectious and noninfectious conditions, and a standardized methodology to assess robustness and cross-reactivity. Applied to 30 published signatures of infection, the analysis showed that signatures were generally robust in detecting viral and bacterial infections in independent data. Asymptomatic and chronic infections were also detectable, albeit with decreased performance. However, many signatures were cross-reactive with unintended infections and aging. In general, the present disclosure found robustness and cross-reactivity to be conflicting objectives, and the present disclosure identified signature properties associated with this trade-off. The data compendium and evaluation framework developed here provide a foundation for the development of signatures for clinical application.

### 3.2. Introduction

The ability to diagnose infectious diseases has a profound impact on global health. Most recently, diagnostic testing for SARS-CoV-2 infection has helped contain the COVID-19 pandemic, lessening the strain on healthcare systems. As a further example, diagnostic technologies that discriminate bacterial from viral infections can inform the prescription of antibiotics. This is a high-stakes clinical decision: if prescribed for bacterial infections, the use of antibiotics substantially reduces mortality (Ferrer et al., 2014), but if prescribed for viral infections, their misuse exacerbates antimicrobial resistance (CDC, 2020).

Standard tests for infection diagnosis involve a variety of technologies including microbial cultures, PCR assays, and antigen-binding assays. Despite the diversity in technologies, standard tests generally share a common design principle, which is to directly quantify pathogen material in patient samples. As a consequence, standard tests have poor detection, particularly early after infection, before the pathogen replicates to detectable levels. For example, PCR-based tests for SARS-CoV-2 infection may miss 60% to 100% of infections within the first few days of infection due to insufficient viral genetic material (Killingley et al., 2022; and Kucirka et al., 2020). Similarly, a study of community acquired pneumonia found that pathogen-based tests failed to identify the causative pathogen in over 60% of patients (Self et al., 2017). To overcome these limitations, new tools for infection diagnosis are urgently needed.

Host transcriptional response assays have emerged as a new paradigm to diagnose infections (Ramilo et al., 2006; Suarez et al., 2015; Sweeney et al., 2016; Tsalik et al., 2021; and Warsinske et al., 2019). Research in the field has produced a variety of host response signatures to detect general viral or bacterial infections as well as signatures for specific pathogens such as influenza virus (Ramilo et al., 2006; Andres-Terre et al., 2015; Davenport et al., 2015; Parnell et al., 2012; Tang et al., 2017; and Zaas et al., 2009). Unlike standard tests that measure pathogen material, these assays monitor changes in gene expression in response to infection (Huang et al., 2011). For example, transcriptional upregulation of IFN response genes may indicate an ongoing viral infection, because these genes take part in the host antiviral response (McNab et al., 2015). Host response assays have a major potential advantage over pathogen-based tests because they may detect an infection even when the pathogen material is undetectable through direct measurements.

Development of host response assays that can be implemented clinically poses new methodological problems. The most challenging problem is identifying the so-called “infection signature” for a pathogen of interest, that is, a set of host transcriptional changes induced in response to that pathogen. Signature performance is characterized along two axes, robustness and cross-reactivity. Robustness is defined as the ability of a signature to detect the intended infectious condition consistently in multiple independent cohorts. Cross-reactivity is defined as the extent to which a signature predicts any condition other than the intended one. To be clinically viable, an infection signature must simultaneously demonstrate high robustness and low cross-reactivity. A robust signature that does not demonstrate low cross-reactivity would detect unintended conditions, such as other infections (e.g., viral signatures detecting bacterial infections) and/or non-infectious conditions involving abnormal immune activation.

The clinical applicability of host response signatures ultimately depends on a rigorous evaluation of their robustness and cross-reactivity properties. However, such an evaluation is a complex task, because it requires integrating and analyzing massive amounts of transcriptional studies involving the pathogen of interest along with a wide variety of other infectious and non-infectious conditions that may cause cross-reactivity. Despite recent progress in this direction (Bodkin et al., 2022; Tsalik et al., 2016; Warsinske et al., 2019), a general framework to benchmark both robustness and cross-reactivity of candidate signatures is still lacking.

Here, the present disclosure establishes a general framework for systematic quantification of robustness and cross-reactivity of a candidate signature, based on a fine-grained curation of massive public data and development of a standardized signature scoring method. Using this framework, the present disclosure demonstrated that published signatures are generally robust but substantially cross-reactive with infectious and non-infectious conditions. Further analysis of 200,000 synthetic signatures identified an inherent trade-off between robustness and cross-reactivity and determined signature properties associated with this trade-off. The disclosed framework, accessible at kleinsteinlab.shinyapps.io/compendium_shiny_app/, lays the foundation for the discovery of signatures of infection for clinical application.

### 3.3. Results

### a Curated Set of Human Transcriptional Infection Signatures

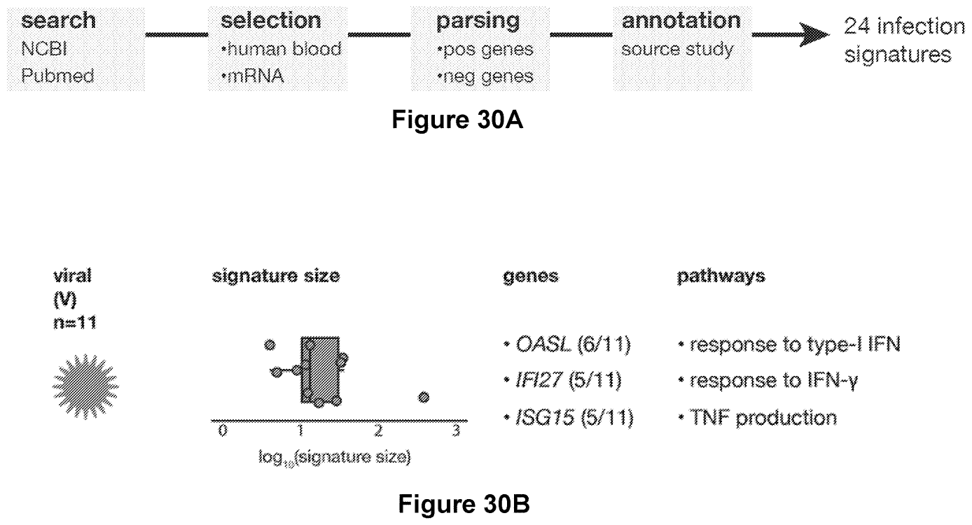

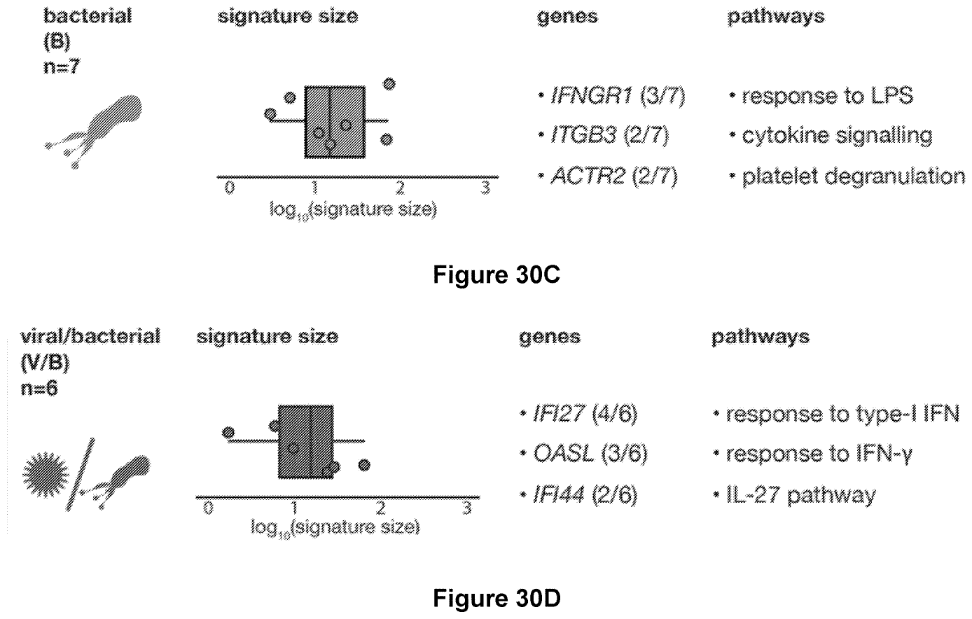

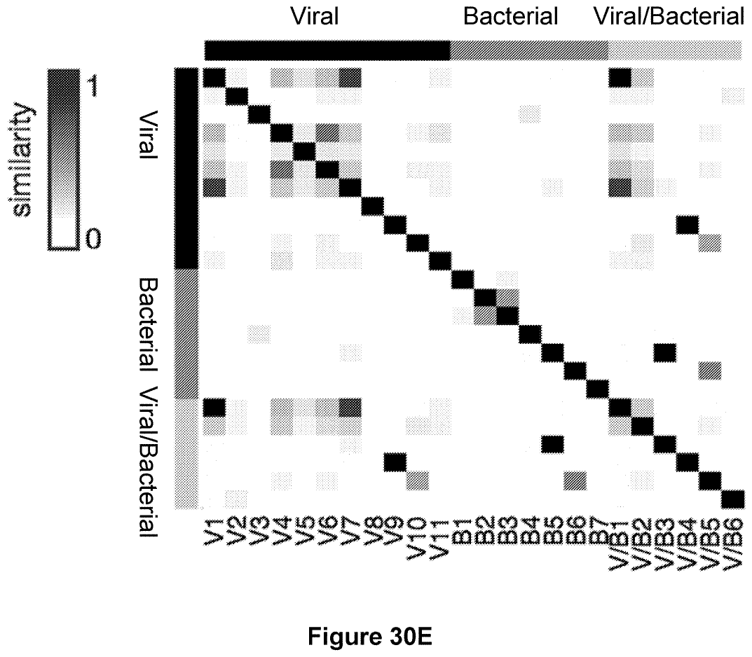

While many transcriptional host response signatures of infection have been published, their robustness and cross-reactivity properties have not been systematically evaluated. To identify published signatures for inclusion in our systematic evaluation, the present disclosure performed a search of NCBI PubMed for publications describing immune profiling of viral or bacterial infections (FIG. 30A). The present disclosure initially focused our curation on general viral or bacterial (rather than pathogen-specific) signatures from human whole blood or peripheral blood mononuclear cells (PBMCs). In some embodiments, the systems and methods of the present disclosure identified 24 signatures that were derived using a wide range of computational approaches, including differential expression analyses (Herberg et al., 2016; Smith et al., 2012, 2013; and Suarez et al., 2015), gene clustering (Hu et al., 2013; and Statnikov et al., 2010), regularized logistic regression (Bhattacharya et al., 2017; Herberg et al., 2016; and Tsalik et al., 2016), and meta-analyses (Andres-Terre et al., 2015; and Sweeney et al., 2016).

The signatures were annotated with multiple characteristics that were needed for the evaluation of performance. The most important characteristic was the intended use of the signatures. The intended use of the included signatures was to detect viral infection (V), bacterial infection (B), or directly discriminate between viral and bacterial infections (V/B). For each signature, the present disclosure recorded a set of genes and a group I vs. group II comparison capturing the design of the signature, where group I was the intended infection type and group II was a control group. For most viral and bacterial signatures, group II was comprised of healthy controls; in a few cases, it was comprised of non-infectious illness controls. For signatures distinguishing viral and bacterial infections (V/B), the present disclosure conventionally took the bacterial infection group as the control group.

In some embodiments, the systems and methods of the present disclosure parsed the genes in these signatures as either ‘positive’ or ‘negative’ based on whether they were up- or down-regulated in the intended group, respectively. In some embodiments, the systems and methods of the present disclosure also manually annotated the PubMed identifiers for the publication in which the signature was reported, accession records to identify discovery datasets used to build each signature, association of the signature with either acute or chronic infection, and additional meta-data related to demographics and experimental design (Table 3.1). Additional details and information regarding Table 3.1 is found at Chawla et al., 2022, “Benchmarking transcriptional host response signatures for infection diagnosis,” Cell Systems, 13(12), pg. 974-988; Supplementary Table 1, which is hereby incorporated by reference in its entirety for all purposes. This curation process identified 11 viral (V) signatures intended to capture transcriptional responses that are common across many viral pathogens, 7 bacterial (B) signatures intended to capture transcriptional responses common across bacterial pathogens, and 6 viral vs. bacterial (V/B) signatures discriminating between viral and bacterial infections.

Viral signatures varied in size between 3 and 396 genes. Several genes appeared in multiple viral signatures. For example, OASL, an interferon-induced gene with antiviral function (Zhu et al., 2014), appeared in 6 of 11 signatures. Enrichment analysis on the pool of viral signature genes showed significantly enriched terms consistent with antiviral immunity, including response to type I interferon (FIG. 30B). Bacterial signatures ranged in size from 2 to 69 genes, and enrichment analysis again highlighted expected pathways associated with antibacterial immunity (FIG. 30C). V/B signatures varied in size from 2 to 69 genes. The most common genes among V/B signatures were OASL and IFI27, both of which were also highly represented viral signature genes, and many of the same antiviral pathways were significantly enriched among V/B signature genes (FIG. 30D). The similarity between viral, bacterial, and V/B signatures was investigated and it was found that many viral signatures shared genes with each other and V/B signatures, but bacterial signatures shared fewer similarities with each other (FIG. 30E). Overall, the curation produced a structured and well-annotated set of transcriptional signatures for systematic evaluation.

### a Compendium of Human Transcriptional Infection Datasets

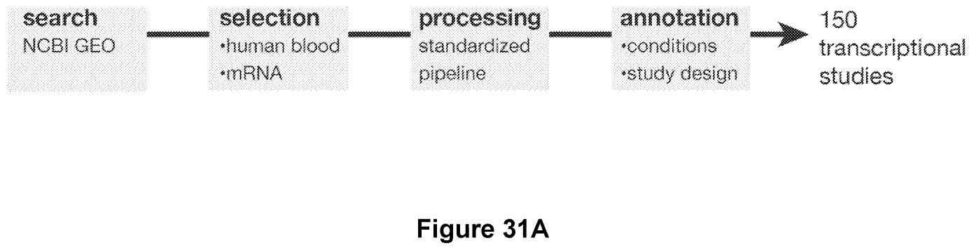

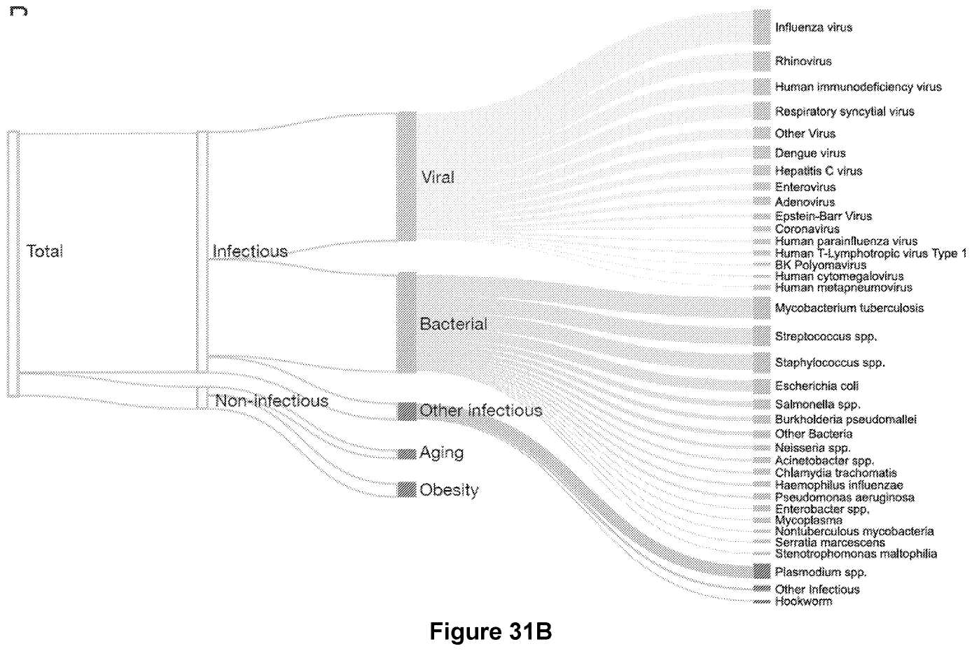

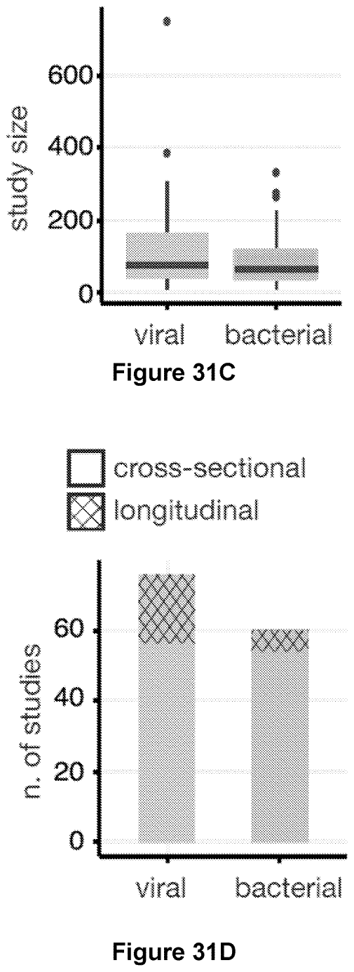

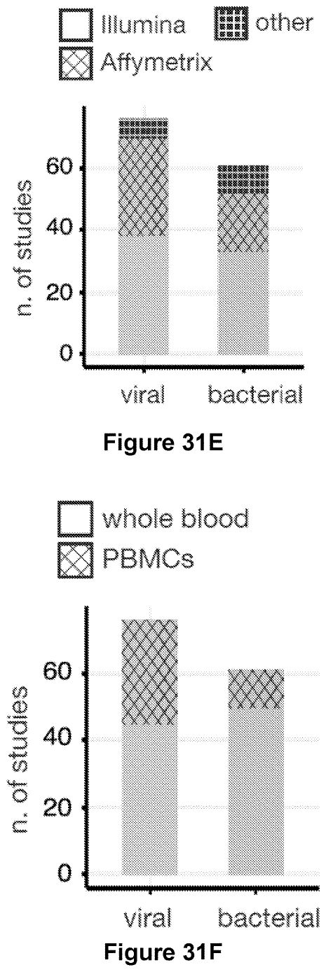

To profile the performance of the curated infection signatures, a large compendium of datasets capturing host blood transcriptional responses to a wide diversity of pathogens was compiled. This was carried out as a comprehensive search in the NCBI Gene Expression Omnibus (GEO) (Barrett et al., 2013) capturing transcriptional responses to in-vivo viral, bacterial, parasitic, and fungal infections in human whole blood or PBMC. Over 8,000 GEO records were screened and 136 transcriptional datasets that met the inclusion criteria (see Methods) were identified. Furthermore, to evaluate whether infection signatures cross-react with non-infectious conditions with documented immunomodulating effects, an additional 14 datasets containing transcriptomes from the blood of aged and obese individuals (Frasca and Blomberg, 2017; and Pereira and Akbar, 2016) were compiled. All datasets were downloaded from GEO and passed through a standardized pipeline. Briefly, the pipeline included: (1) uniform pre-processing of raw data files where possible, (2) remapping of available gene identifiers to Entrez Gene IDs, and (3) detection of outlier samples (Kauffmann et al., 2009). In aggregate, the present disclosure compiled, processed and annotated 150 datasets to include in our data compendium (FIG. 31A, Table 3.2, see Methods for details). Additional details and information regarding Table 3.2 is found Chawla et al., 2022, “Benchmarking transcriptional host response signatures for infection diagnosis,” Cell Systems, 13(12), pg. 974-988; Supplementary Table 2, which is hereby incorporated by reference in its entirety for all purposes.

The compendium datasets showed dramatic variability in study design, sample composition, and available metadata necessitating annotation both at the study level and at the finer-grained sample level. Datasets followed either cross-sectional study designs, where individual subjects were profiled once for a snapshot of their infection, or longitudinal study designs in which individual subjects were profiled at multiple time points over the course of an infection. For longitudinal datasets, the present disclosure also recorded subject identifiers and labeled time points. Many datasets contained multiple subgroups, each profiling infection with a different pathogen. Detailed review of the clinical methods and metadata for each study enabled annotation of individual samples with infectious class (e.g., viral, bacterial) and causative pathogen. For clinical variables, whether datasets profiled acute or chronic infections were manually recorded according to the authors and annotated symptom severity when available. This information was further supplemented with biological sex, inferred computationally (see Methods). In total, 16,173 infection and control samples were annotated in a consistent way, capturing host responses to viral, bacterial, and parasitic infections. An additional 932 samples from aging and obesity datasets including young and lean controls respectively were similarly annotated. In aggregate, a broad range of more than 35 unique pathogens and non-infectious conditions were captured (FIG. 31B).

Most of the compendium datasets were composed of viral and bacterial infection response profiles. Several technical factors that may bias the signature performance evaluation across these categories were examined. Datasets profiling viral infections and datasets profiling bacterial infections contained similar numbers of samples, with median samples sizes of 75.5 and 63 respectively, though the largest viral studies contained more samples than the largest bacterial studies (FIG. 31C). The number of cross-sectional studies was also nearly identical for both viral and bacterial infection datasets, but the compendium contained 20 viral longitudinal datasets (35% of viral) compared to 6 bacterial longitudinal datasets (10% of bacterial) (FIG. 31D). The distribution of platforms used to generate viral and bacterial infection datasets was examined. It was found that gene expression was measured most commonly using Illumina platforms followed by Affymetrix for both viral and bacterial datasets (FIG. 31E). The frequency of whole blood and PBMC samples in the compendium was also examined (FIG. 31F). Systematic differences in the viral and bacterial datasets within the compendium were not identified, and therefore these differences were not expected to impact the interpretation of the signature evaluations.

### Establishing a General Framework for Signature Evaluation

In some embodiments, the systems and methods of the present disclosure sought to quantify two measures of performance for all curated signatures: (1) robustness, the ability of a signature to predict its target infection in independent datasets not used for signature discovery, and (2) cross-reactivity, which were quantified as the undesired extent to which a signature predicts unrelated infections or conditions. An ideal signature would demonstrate robustness but not cross-reactivity, e.g., an ideal viral signature would predict viral infections in independent datasets but would not be associated with infections caused by pathogens such as bacteria or parasites.

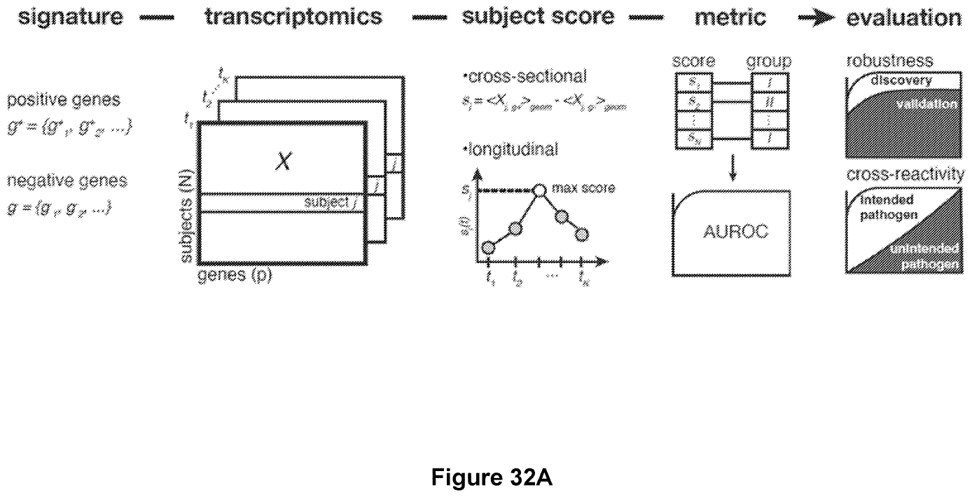

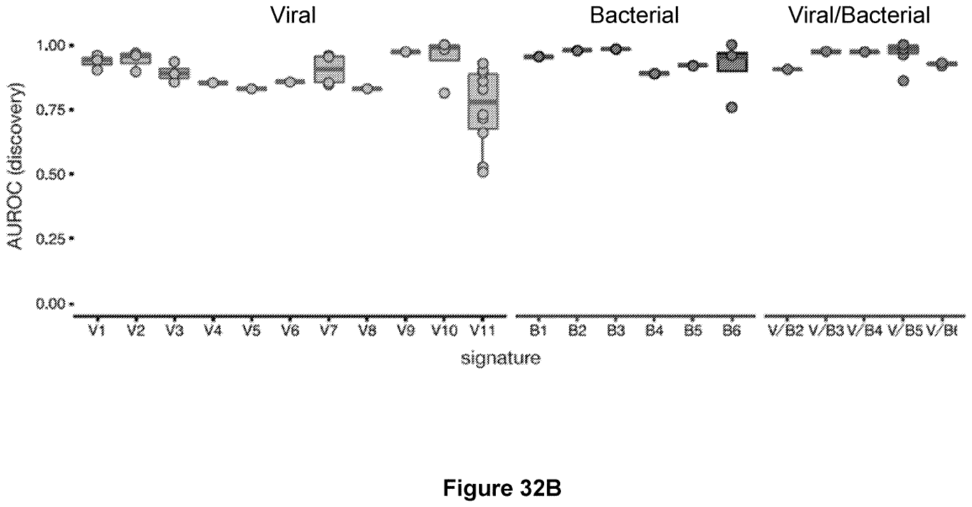

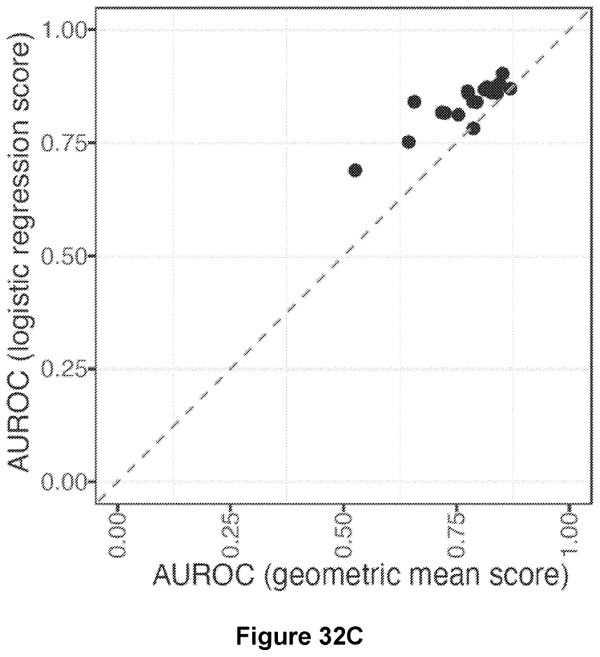

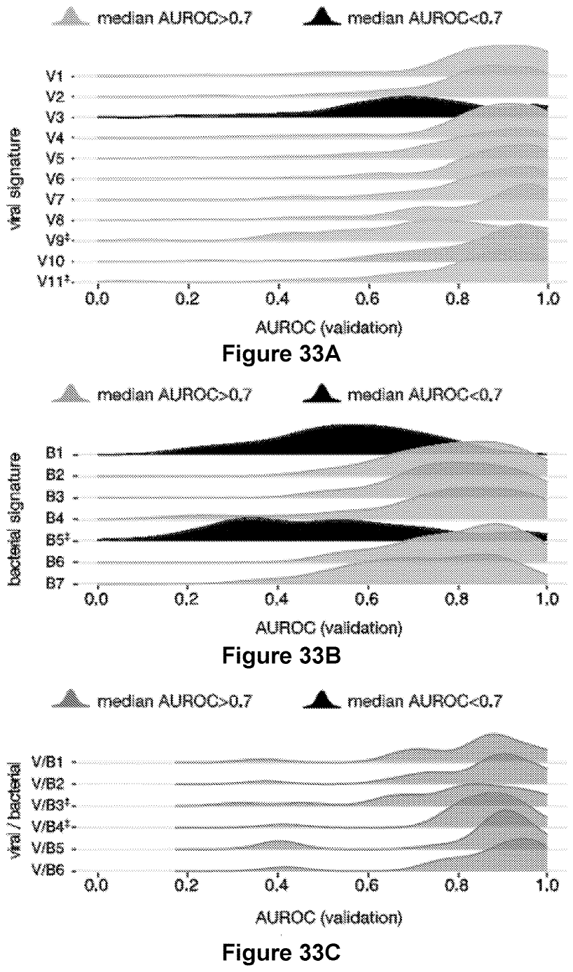

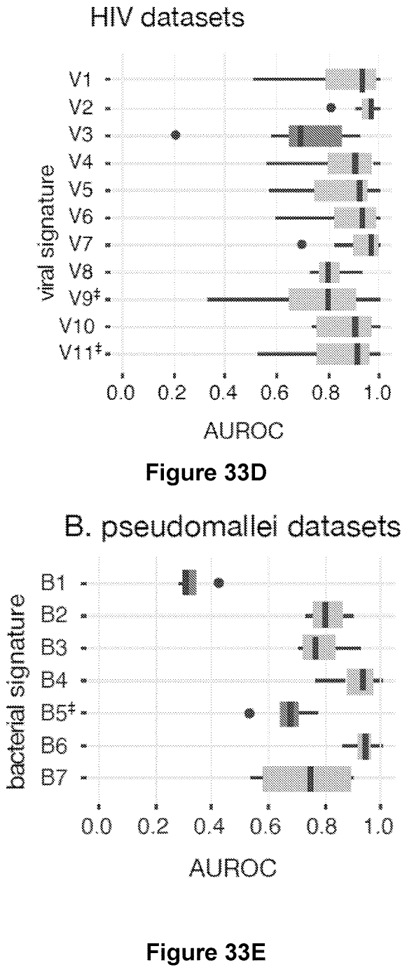

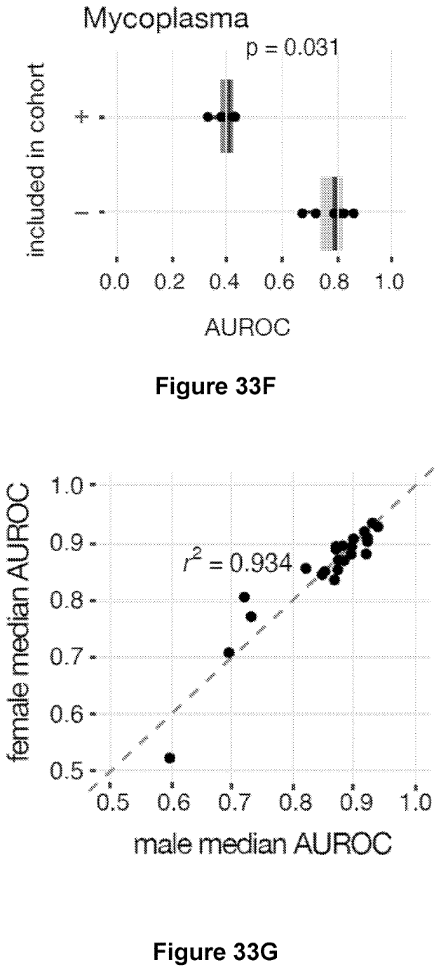

To score each signature in a standardized way, the present disclosure leveraged the geometric mean scoring approach described in (Haynes et al., 2016). For each signature (e.g. a set of positive genes and an optional set of negative genes), the present disclosure calculated its sample score from log-transformed expression values by taking the difference between the geometric mean of positive signature gene expression values and the geometric mean of negative signature gene expression values. For cross-sectional study designs, this generates a single signature score for each subject, but for longitudinal study designs, this approach produces a vector of scores across time points for each subject. The scores at different time points can vary dramatically as the transcriptional program underlying an immune response changes over the course of an infection (Andres-Terre et al., 2015; Huang et al., 2011; Sweeney et al., 2015). In this case, the present disclosure chose the maximally discriminative time point, so that a signature is considered robust if it can detect the infection at any time point, but also considered cross-reactive if it would produce a false positive call at any time point (see Methods). These subject scores were then used to quantify signature performance as the area under a receiver operator characteristic curve (AUROC) associated with each group comparison. The approach is advantageous because it is computationally efficient and model-free. The model-free property presents an advantage over parameterized models because it does not require transferring or re-training model coefficients between datasets. Overall, this framework enables the evaluation of the performance of all signatures in a standardized and consistent way in any dataset (FIG. 32A).

The framework was assessed by computing each signature's performance on the datasets used originally for its discovery. If the approach is valid, signatures evaluated in their own discovery datasets should perform well, generating AUROCs close to 1. Consistent with this reasoning, it was found that each signature strongly predicted infections in its own discovery datasets: the lowest observed median AUROC was 0.78 among viral signatures, 0.82 among bacterial signatures, and 0.90 among V/B signatures (FIG. 32B). The choice of geometric mean scoring was also specifically evaluated and it was found that the performance of this scoring method for all signatures was highly correlated with logistic regression (Bhattacharya et al., 2017; Herberg et al., 2016; Tsalik et al., 2016), a popular alternative approach (see Methods and FIG. 32C). These results highlighted that while individual signatures were developed using many different methods, signatures can be reliably evaluated using a standardized framework built on geometric mean scoring.

### Existing Signatures of Bacterial and Viral Infection are Generally Robust

Having established a common framework for evaluating signatures the present disclosure next investigated the robustness of all curated signatures. Each signature in our compendium was first evaluated on every non-discovery (e.g., independent) dataset profiling intended pathogen responses and healthy controls. For example, all signatures of viral infection were evaluated on datasets that profiled viral pathogens and healthy controls. In some embodiments, the systems and methods of the present disclosure used the median AUROC threshold of 0.7 for robustness determination (see Methods). Overall, the present disclosure found that 10 out of 11 viral signatures, 5 out of 7 bacterial signatures, and all 6 V/B signatures achieved a median AUROC greater than 0.70 in predicting infections in independent data (FIGS. 33A-33C, Table 3.3). Additional details and information regarding Table 3.3 is found at Chawla et al., 2022, “Benchmarking transcriptional host response signatures for infection diagnosis,” Cell Systems, 13(12), pg. 974-988; Supplementary Table 3, which is hereby incorporated by reference in its entirety for all purposes. Additionally, because some signatures were derived using non-infectious illness controls (e.g., systemic inflammatory response syndrome), the present disclosure characterized viral and bacterial signature performance in datasets that profiled this contrast (Sampson et al., 2017; and Tsalik et al., 2016). In this evaluation, 9 out of 11 viral signatures and 2 out of 7 bacterial signatures achieved a median AUROC greater than 0.70 (FIGS. 33J and K; Table 3.3), suggesting that bacterial but not viral signatures were sensitive to the control group used for signature evaluation. In some embodiments, the systems and methods of the present disclosure categorized a signature as robust if its median AUROC in either set of independent data (e.g., vs. healthy or non-infectious illness controls) was greater than 0.70, indicating strong predictive performance. Overall, the present disclosure identified 10 viral, 6 bacterial and all 6 V/B signatures that were robust.

Viral and bacterial signatures also robustly detected infections caused by pathogens in the same class (e.g., viral or bacterial) that were not included among discovery datasets. For example, all 10 robust viral signatures detected infections caused by HIV (median AUROC>0.8, FIG. 33D), while this pathogen was not included among the discovery datasets. Similarly, all robust bacterial signatures detected infections caused by B. pseudomallei (FIG. 33E), while this pathogen was not included among the discovery datasets. These results suggest strong conservation of transcriptional programs underlying immune responses against a broad array of viruses and bacteria.

While signatures were discovered using different blood subsets and transcriptional profiling platforms, signature robustness was not strongly influenced by these factors. Signature performance in datasets profiling whole blood was strongly correlated with performance in datasets profiling PBMCs (r=0.96). Similarly, signature performance in datasets generated using Illumina microarray platforms was strongly correlated with performance in datasets generated using Affymetrix platforms (r=0.91). See Figure S2E of Chawla et al., 2022 “Benchmarking transcriptional host response signature for infection diagnosis,” Cell Systems 13, 974-988, which is hereby incorporated by reference.

There were a few datasets in the compendium where most signatures performed poorly, generating a dataset median AUROC less than or equal to 0.50. For viral signatures, 2 such outlier datasets were observed (GSE85599 and GSE59312) characterizing immune responses to acute and chronic Epstein-Barr virus (EBV) infection and chronic hepatitis C virus (HCV) infection, respectively. See FIGS. S3A and S3B of Chawla et al., 2022 “Benchmarking transcriptional host response signature for infection diagnosis,” Cell Systems 13, 974-988, which is hereby incorporated by reference. For bacterial signatures one such outlier dataset was observed (GSE625625), characterizing the response to Mycobacterium tuberculosis infection (TB). See Figures S3C of Chawla et al., 2022 “Benchmarking transcriptional host response signature for infection diagnosis,” Cell Systems 13, 974-988, which is hereby incorporated by reference. Whether performance loss in the outlier datasets was due to the causative pathogens or to technical factors of the datasets was considered. To address this, the signature performance in additional datasets profiling the same causative pathogens (EBV, HCV and TB) were analyzed. It was found that both viral and bacterial signatures showed robust performance in these additional datasets, demonstrating that performance loss in the outlier datasets was likely due to technical factors rather than the pathogen. See FIGS. S3A-S3C of Chawla et al., 2022 “Benchmarking transcriptional host response signature for infection diagnosis,” Cell Systems 13, 974-988, which is hereby incorporated by reference. V/B signatures performed poorly in one outlier dataset profiling viral and bacterial pediatric pneumonia (GSE103119). While multiple datasets in the compendium profiled pediatric pneumonia, this outlier dataset was unique in its inclusion of Mycoplasma, a bacterium that lacks a cell wall, as the causative pathogen. To assess whether V/B signatures perform poorly for Mycoplasma infection specifically, this pathogen was removed from the dataset and the signature assessment was repeated. The performance of signatures for non-Mycoplasma bacterial infections was significantly improved (p=0.031, FIG. 33F). Thus, the poor V/B signature performance in this case likely reflects a unique biological response for Mycoplasma that more closely resembles a viral infection.

### Multiple Infection Characteristics Modulate Signature Robustness

A number of factors can influence transcriptional profiles of infection and therefore signature performance, including subject demographics and clinical state (Andres-Terre et al., 2015; Huang et al., 2011). Although the availability and structure of metadata fields varied between datasets, three variables of interest were systematically evaluated: sex, acute versus chronic infection characterization, and disease severity. Due to data limitations, the analysis of infection characterization and severity was conducted only for viral signatures. In particular, to address the effect of severity, a single challenge study with three datasets was analyzed, each of which profiled symptomatic and asymptomatic viral infections by human rhinovirus (hRV) or influenza virus H1N1 (Gene Expression Omnivus: GSE73072). In the analysis of these datasets, only subjects with evidence of viral shedding were included, to ensure that results were not due to lack of productive infection.

It was found that signature performance was: (1) strongly correlated between males and females (FIG. 33G), and not significantly different in the two groups (paired t-test, p>0.05) (FIG. S4A of Chawla et al., 2022 “Benchmarking transcriptional host response signature for infection diagnosis,” Cell Systems 13, 974-988, which is hereby incorporated by reference); (2) robust for both acute and chronic infections, albeit significantly lower in the latter case (FIG. 33H, p<0.001); (3) robust for both symptomatic and asymptomatic infections, but significantly lower in the second group (p<0.009, FIG. 33I). See also FIG. S4B of Chawla et al., 2022 “Benchmarking transcriptional host response signature for infection diagnosis,” Cell Systems 13, 974-988, which is hereby incorporated by reference. Taken together, this analysis identified chronic versus acute characterization and severity, but not sex, as determinants that significantly affect signature performance.

Infection timing may also play an important role in modulating signature robustness (Sweeney et al., 2015). While time of pathogen exposure relative to sample collection is unknown for nearly all subjects in the disclosed compendium, eight datasets profiled healthy volunteers who were challenged with exposure to live respiratory viruses (Davenport et al., 2015; Liu et al., 2016). To investigate the effect of timing on signature robustness in these datasets, each time point post-infection was treated as an independent cross-sectional study and computed signature AUROCs. See FIG. S5 of Chawla et al., 2022 “Benchmarking transcriptional host response signature for infection diagnosis,” Cell Systems 13, 974-988, which is hereby incorporated by reference. Robust viral signatures achieved median AUROCs greater than 0.70 between 34- and 77-hours post-infection and remained robust for several days through the end of each study. This analysis identified an initial, undetectable infection period followed by a prolonged, robust detection window for acute viral infection.

Nearly all Infection Signatures are Cross-Reactive with Infectious and Non-Infectious Conditions

The cross-reactivity of the 22 infection signatures found to be robust were assessed. Signature cross-reactivity was estimated with the same evaluation framework used to assess robustness, but now applied to data from infectious and non-infectious conditions for which the signature was not intended (see Methods). A signature was considered cross-reactive with an unintended condition if the corresponding median AUROC was greater than 0.60 (see Methods).

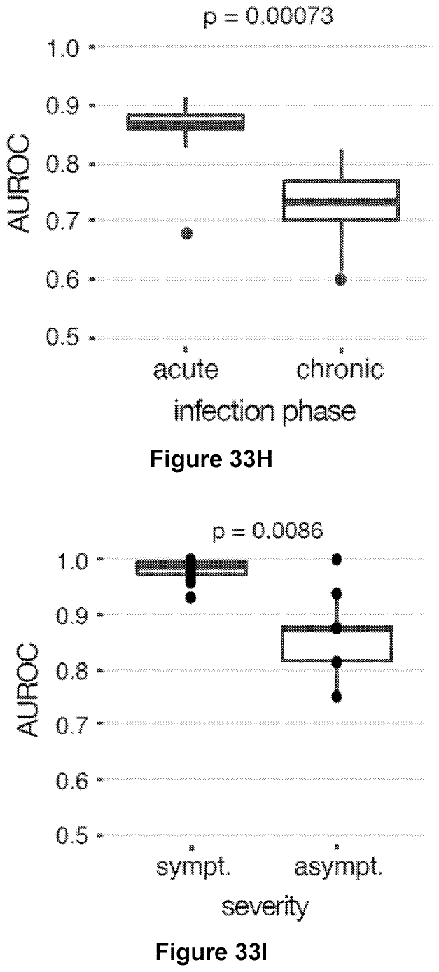

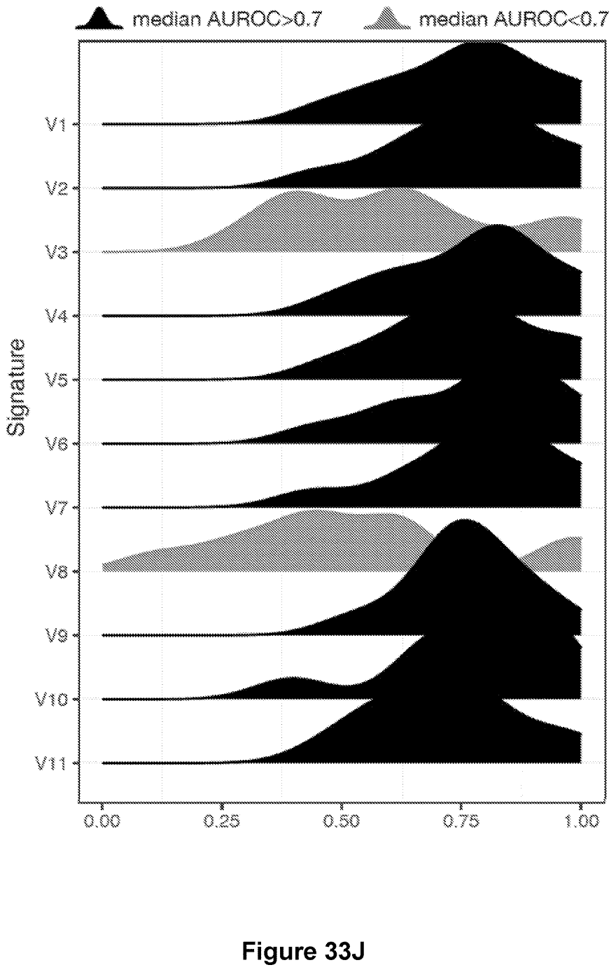

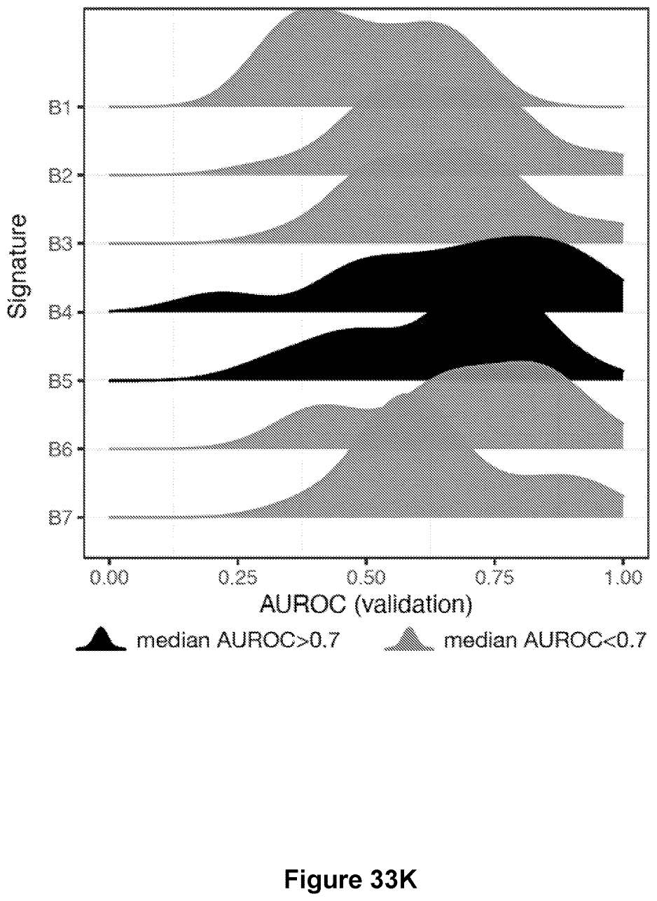

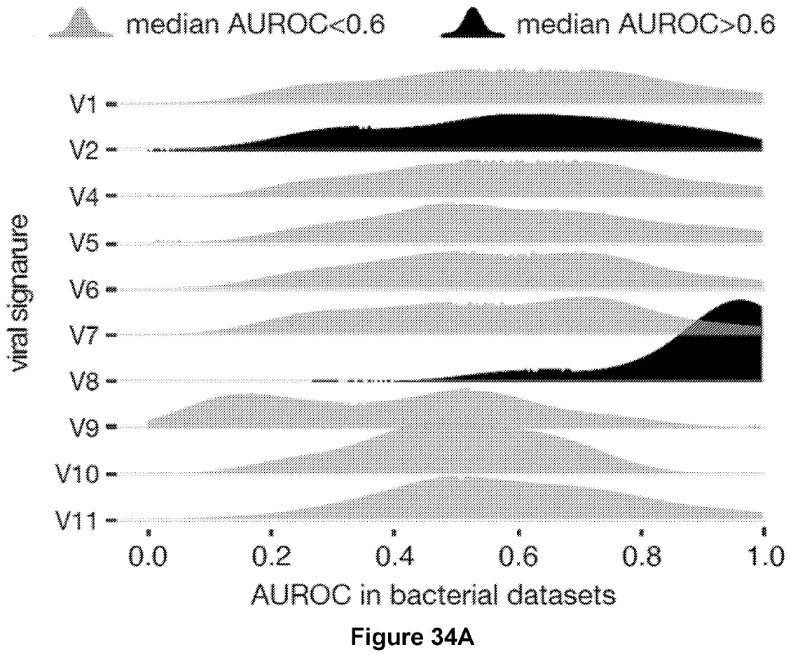

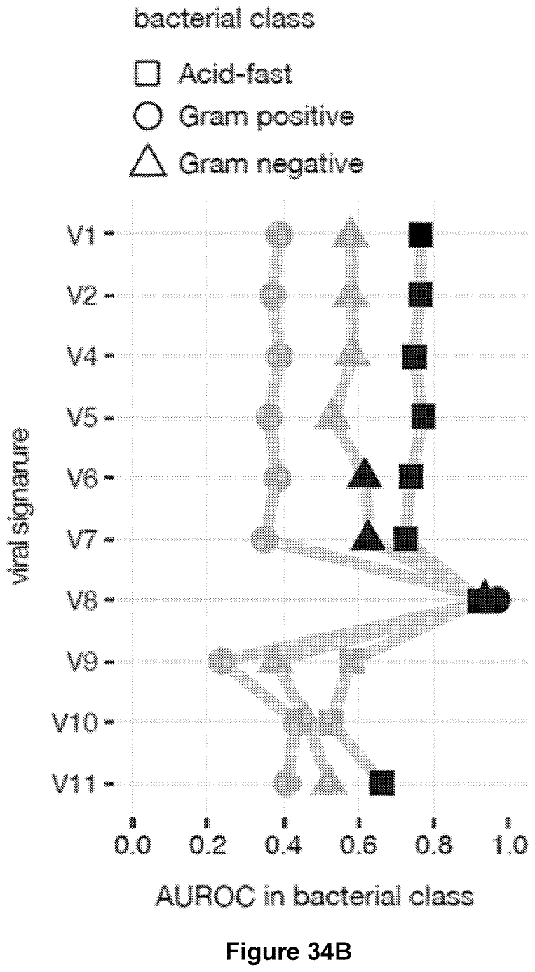

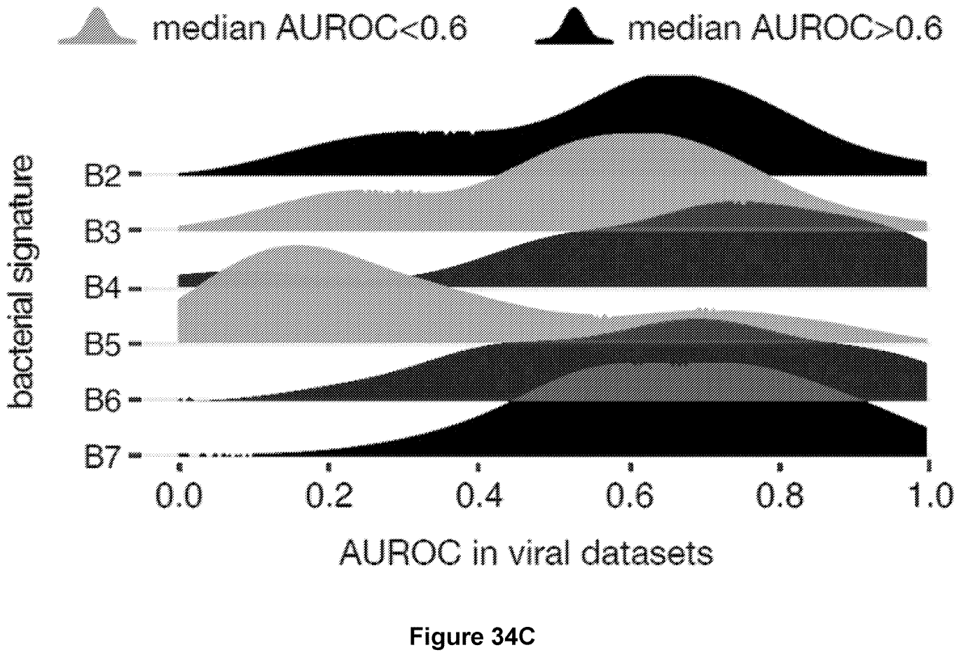

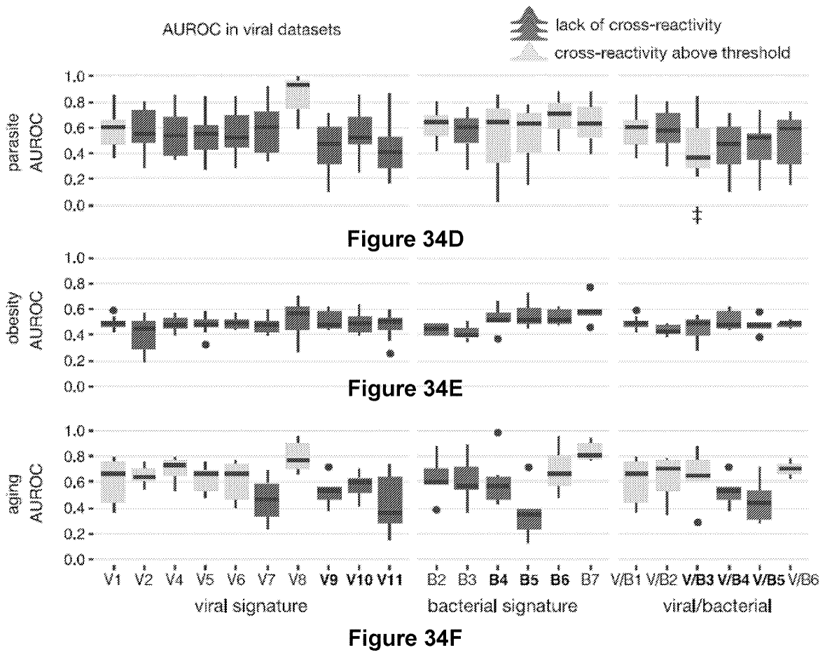

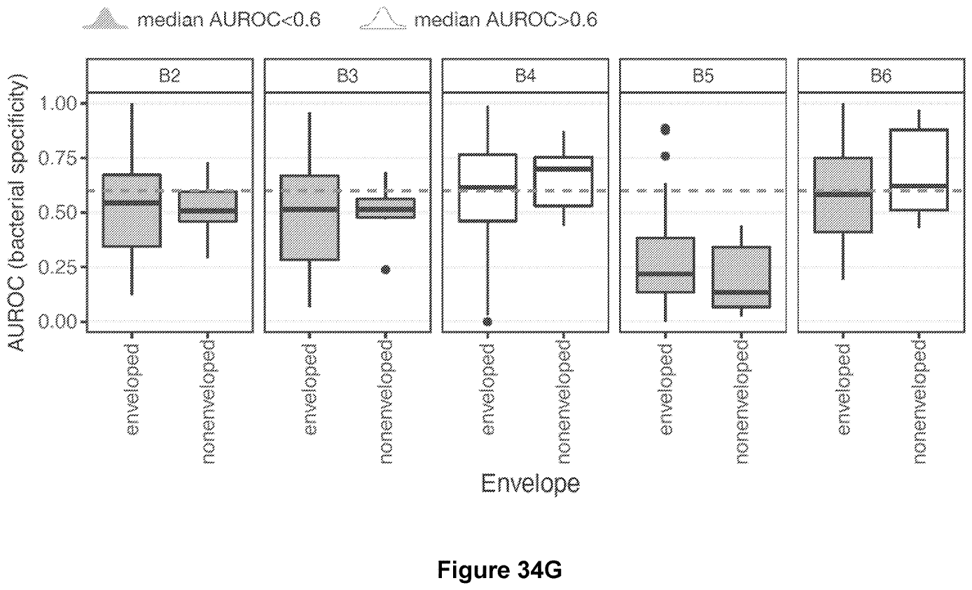

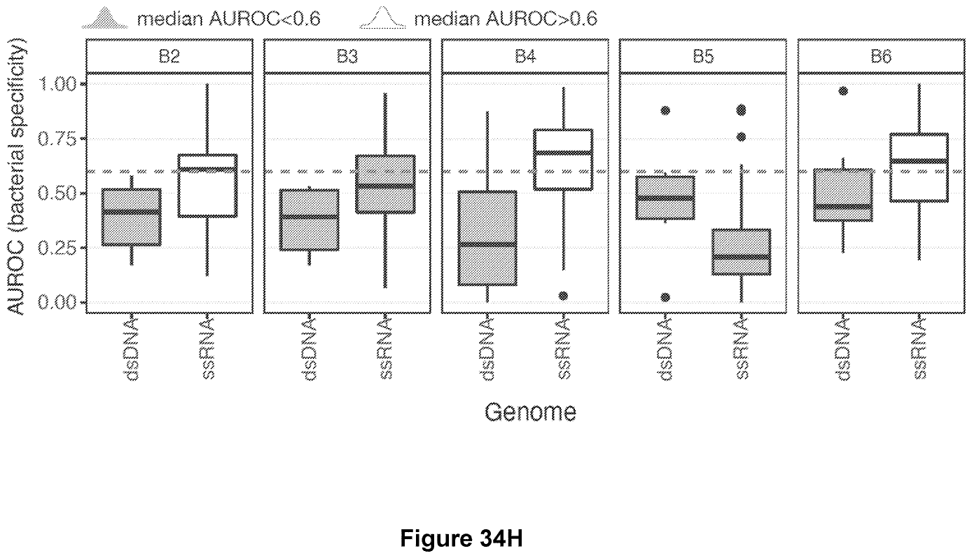

First, the cross-reactivity of viral signatures was examined with respect to bacterial infections. It was found that V2 and V8 were cross-reactive (FIG. 34A). All viral signatures showed a wide range of cross-reactivity values. Whether this variability might reflect the variability in the classes of bacterial pathogens was considered. The bacterial pathogens were classified based on cell wall characteristics as gram-positive, gram-negative, and acid-fast bacteria, and quantified cross-reactivity separately for these classes (FIG. 34B). Most signatures cross-reacted with acid-fast bacteria (8 out of 10 signatures, median AUROCs between 0.66 and 0.92), a class that includes Mycobacterium tuberculosis. In contrast, only 3 of 10 signatures cross-reacted with gram negative bacteria, and only one with gram positive bacteria. Overall, only two signatures, V9 and V10, did not cross-react with any bacterial pathogen class. These results demonstrate that viral signatures' cross-reactivity depends on pathogen cell-wall characteristics and is highest for acid-fast bacteria. Despite this, it is possible to generate viral signatures that are not cross-reactive with any bacterial class.

Second, the present disclosure examined the extent to which bacterial signatures predicted viral infections. It was found that four of the six robust bacterial signatures were cross-reactive with viral infections (FIG. 34C). Similar to above, a wide range of cross-reactivity values were observed. To better understand this variability, viral pathogens were classified based on the presence or absence of a viral envelope and on viral genome characteristics and cross-reactivity was quantified separately for these groups. Cross-reactivity did not vary based on the presence of a viral envelope (FIG. 34G). In contrast, a trend indicating greater cross-reactivity among single-stranded RNA viruses compared to double-stranded DNA viruses was observed (FIG. 34H).

As a final test of cross-reactivity with infectious conditions, signature cross-reactivity with parasitic infections was quantified. In addition to the viral and bacterial signatures, it was also possible to measure the cross-reactivity of the V/B signatures in this evaluation because parasites were an unintended pathogen class for these signatures. Most bacterial signatures, but few viral or V/B signatures, showed cross-reactivity with parasitic infections (FIG. 34 D). See also the color version of FIG. 34D, which is FIG. 5D of Chawla et al., 2022 “Benchmarking transcriptional host response signature for infection diagnosis,” Cell Systems 13, 974-988, which is hereby incorporated by reference.

Non-infectious factors, such as obesity and aging, are associated with altered immune states that may produce false positive signals for infectious signatures (Frasca and Blomberg, 2017; and Pereira and Akbar, 2016). The cross-reactivity of viral, bacterial, and V/B signatures were evaluated with these non-infectious conditions (see Methods for clinical definitions, cohort accessions in Table 3.2). It was found that viral, bacterial, and V/B signatures did not cross react with obesity (FIG. 34E, Table 3.3). In contrast, 6 of 10 viral, 2 of 6 bacterial, and 4 of 6 V/B signatures were cross-reactive with aging (FIG. 34F, Table 3.3). In these cases, the signatures falsely detected an infection signal in healthy, older adults relative to young adults. Among the 10 signatures which were not cross-reactive with aging, 7 were derived from cohorts containing both pediatric and adult subjects spanning an age range greater than 50 years (FIG. 34F, Table 3.1). Additional details and information regarding Tables 3.1, 3.2, and 3.3 is found at Chawla et al., 2022, “Benchmarking transcriptional host response signatures for infection diagnosis,” Cell Systems, 13(12), pg. 974-988; Supplementary Tables 1, 2, and 3, which is hereby incorporated by reference in its entirety for all purposes.

### Single-Pathogen Influenza Signatures are Robust but Cross-Reactive

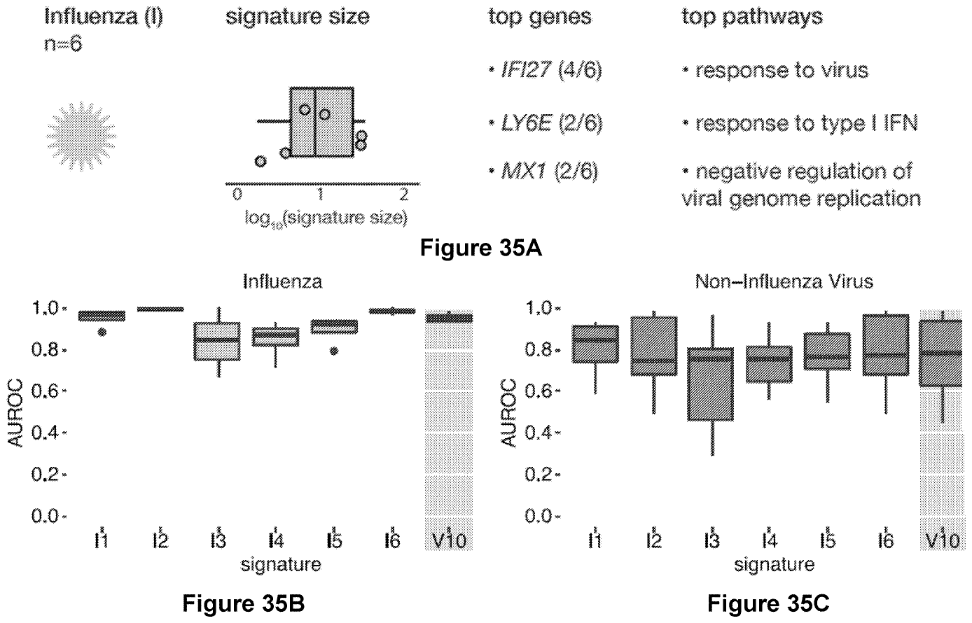

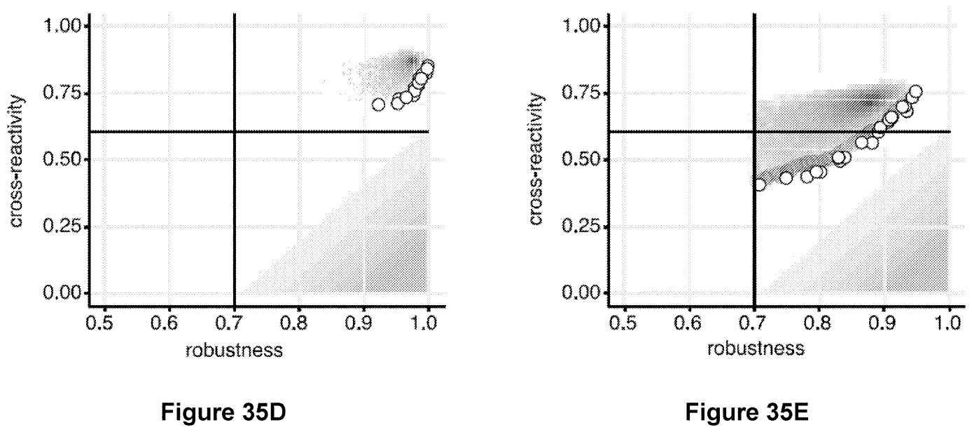

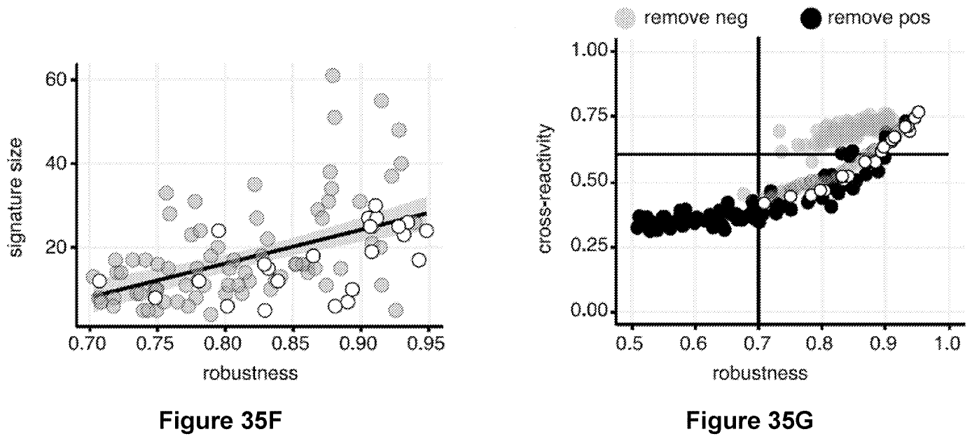

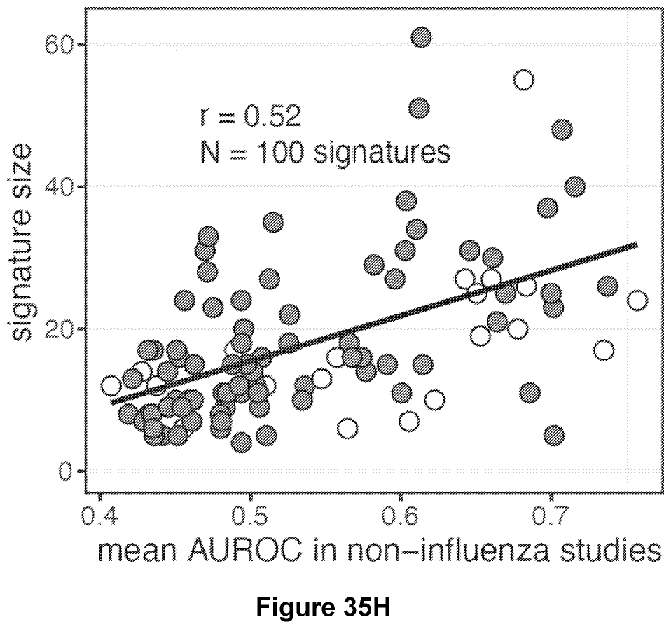

The previous analysis focused on generic signatures of infection by a pathogen class, such as viral signatures. In some embodiments, the systems and methods of the present disclosure next focused on signatures of infection by a single pathogen and chose to study the influenza virus, because influenza causes a large, worldwide morbidity and mortality burden (Juliano et al., 2018). Influenza was also the most abundant viral pathogen in our data compendium, with profiles from infected subjects reported in more than 30 datasets. A targeted search of NCBI PubMed identified 6 published signatures (I1-I6, Table 3.4) containing between 1 and 27 genes. Additional details and information regarding Table 3.4 is found at Chawla et al., 2022, “Benchmarking transcriptional host response signatures for infection diagnosis,” Cell Systems, 13(12), pg. 974-988; Supplementary Table 4, which is hereby incorporated by reference in its entirety for all purposes. These signatures included many interferon response genes that were also found in generic viral signatures (FIG. 35A, Table 3.4) and were significantly enriched for terms such as ‘response to type I interferon’ and ‘response to virus’. Unlike general viral signatures, none of the curated influenza signatures were derived using non-infectious illness controls (e.g., sterile inflammatory response syndrome). The evaluation therefore focused on discriminating influenza virus infection from healthy control samples.

To evaluate the performance of influenza signatures, the present disclosure used as reference the best performing generic viral signature (V10). Compared with this generic viral signature, it was expected that influenza signatures would be at least as robust, but substantially less cross-reactive with viral infections not caused by influenza. It was found that all influenza signatures robustly discriminated influenza infection from healthy controls, with median AUROCs ranging from 0.82 to 0.99, comparable with V10 (FIG. 35B). However, all influenza signatures cross-reacted with non-influenza respiratory viral infections (such as hRV and RSV infection) with median AUROCs between 0.74 and 0.84 (Table 3.4). These values were comparable to those observed with the generic viral signature V10, confirming that influenza signatures lack influenza specificity (FIG. 35C). Further evaluation of cross-reactivity showed that only two of six influenza signatures (I3 and 15) did not cross-react with bacterial infections, parasite infections, or aging. See FIGS. S7A-S7D of Chawla et al., 2022, “Benchmarking transcriptional host response signatures for infection diagnosis,” Cell Systems, 13(12), pg. 974-988, which is hereby incorporated by reference. Thus, while these influenza signatures were robust, they were cross-reactive with both infectious and non-infectious conditions.

### Analysis of Influenza Signatures Demonstrates a Trade-Off Between Robustness and Cross-Reactivity

An investigation as whether it was possible to reduce the cross-reactivity of influenza infection signatures was made. The meta-analysis based signature derivation approach used to develop V 10 was followed, a general viral signature that was not cross-reactive. A meta-analysis of 10 datasets profiling influenza infection and healthy control samples was performed to identify an initial set of 124 differentially expressed candidate genes (data accessions in Tables 3.5, gene identifiers in Table 3.6, see Methods).

TABLE 3.5

| | | | | |
|---|---|---|---|---|
| Accession | Usage | Contrast | Pathogen | Tissue |

Accessions for Flu versus Healthy

| | | | | |
|---|---|---|---|---|
| GSE42026 | Discovery | Influenza | Influenza virus | whole |
| | | virus vs. | | blood |
| | | healthy | | |
| GSE61754 | Discovery | Influenza | Influenza virus | whole |
| | | virus vs. | | blood |
| | | healthy | | |
| GSE68310 | Discovery | Influenza | Influenza virus | PBMCs |
| | | virus vs. | | |
| | | healthy | | |
| GSE114466 | Discovery | Influenza | Influenza virus | whole |
| | | virus vs. | | blood |
| | | healthy | | |
| GSE20346 | Discovery | Influenza | Influenza virus | whole |
| | | virus vs. | | blood |
| | | healthy | | |
| GSE21802 | Discovery | Influenza | Influenza virus | whole |
| | | virus vs. | | blood |
| | | healthy | | |
| GSE29385 | Discovery | Influenza | Influenza virus | whole |
| | | virus vs. | | blood |
| | | healthy | | |
| GSE101702 | Discovery | Influenza | Influenza virus | whole |
| | | virus vs. | | blood |
| | | healthy | | |
| GSE34205 | Discovery | Influenza | Influenza virus | PBMCs |
| | | virus vs. | | |
| | | healthy | | |
| GSE61821 | Discovery | Influenza | Influenza virus | whole |
| | | virus vs. | | blood |
| | | healthy | | |
| GSE82050 | Validation | Influenza | Influenza virus | whole |
| | (robustness) | virus vs. | | blood |
| | | healthy | | |
| GSE111368 | Validation | Influenza | Influenza virus | whole |
| | (robustness) | virus vs. | | blood |
| | | healthy | | |
| GSE40012 | Validation | Influenza | Influenza virus | whole |
| | (robustness) | virus vs. | | blood |
| | | healthy | | |
| GSE6269 | Validation | Influenza | Influenza virus | PBMCs |
| | (robustness) | virus vs. | | |
| | | healthy | | |
| GSE42026 | Validation | Non- | Respiratory syncytial virus | whole |
| | (cross- | influenza | | blood |
| | reactivity) | virus vs. | | |
| | | healthy | | |
| GSE68310 | Validation | Non- | Coronavirus (seasonal); | PBMCs |
| | (cross- | influenza | Enterovirus; | |
| | reactivity) | virus vs. | Respiratory syncytial virus; | |
| | | healthy | Rhinovirus | |
| GSE29385 | Validation | Non- | Rhinovirus | whole |
| | (cross- | influenza | | blood |
| | reactivity) | virus vs. | | |
| | | healthy | | |
| GSE67059 | Validation | Non- | Rhinovirus | whole |
| | (cross- | influenza | | blood |
| | reactivity) | virus vs. | | |
| | | healthy | | |
| GSE67059 | Validation | Non- | Rhinovirus | whole |
| | (cross- | influenza | | blood |
| | reactivity) | virus vs. | | |
| | | healthy | | |
| GSE103842 | Validation | Non- | Respiratory syncytial virus | whole |
| | (cross- | influenza | | blood |
| | reactivity) | virus vs. | | |
| | | healthy | | |
| GSE34205 | Validation | Non- | Respiratory syncytial virus | PBMCs |
| | (cross- | influenza | | |
| | reactivity) | virus vs. | | |
| | | healthy | | |
| GSE97741 | Validation | Non- | Respiratory syncytial virus; | whole |
| | (cross- | influenza | Rhinovirus | blood |
| | reactivity) | virus vs. | | |
| | | healthy | | |
| GSE103119 | Validation | Non- | Adenovirus; Bocavirus; | whole |
| | (cross- | influenza | Coronavirus; | blood |
| | reactivity) | virus vs. | Human metapneumovirus; | |
| | | healthy | Human parainfluenza virus; | |
| | | | Respiratory syncytial virus; | |
| | | | Rhinovirus | |
| GSE38900 | Validation | Non- | Respiratory syncytial virus | whole |
| | (cross- | influenza | | blood |
| | reactivity) | virus vs. | | |
| | | healthy | | |
| GSE38900 | Validation | Non- | Respiratory syncytial virus | whole |
| | (cross- | influenza | | blood |
| | reactivity) | virus vs. | | |
| | | healthy | | |
| GSE69606 | Validation | Non- | Respiratory syncytial virus | PBMCs |
| | (cross- | influenza | | |
| | reactivity) | virus vs. | | |
| | | healthy | | |
| GSE42026 | Discovery | Influenza | Influenza virus | whole |
| | | virus vs. | | blood |
| | | healthy | | |

Accessions for flu versus non-flu

| | | | | |
|---|---|---|---|---|
| GSE42026 | Discovery | Influenza | Influenza virus | whole |
| | | virus vs. | | blood |
| | | healthy | | |
| GSE61754 | Discovery | Influenza | Influenza virus | whole |
| | | virus vs. | | blood |
| | | healthy | | |
| GSE68310 | Discovery | Influenza | Influenza virus | PBMCs |
| | | virus vs. | | |
| | | healthy | | |
| GSE114466 | Discovery | Influenza | Influenza virus | whole |
| | | virus vs. | | blood |
| | | healthy | | |
| GSE20346 | Discovery | Influenza | Influenza virus | whole |
| | | virus vs. | | blood |
| | | healthy | | |
| GSE21802 | Discovery | Influenza | Influenza virus | whole |
| | | virus vs. | | blood |
| | | healthy | | |
| GSE29385 | Discovery | Influenza | Influenza virus | whole |
| | | virus vs. | | blood |
| | | healthy | | |
| GSE101702 | Discovery | Influenza | Influenza virus | whole |
| | | virus vs. | | blood |
| | | healthy | | |
| GSE34205 | Discovery | Influenza | Influenza virus | PBMCs |
| | | virus vs. | | |
| | | healthy | | |
| GSE61821 | Discovery | Influenza | Influenza virus | whole |
| | | virus vs. | | blood |
| | | healthy | | |
| GSE82050 | Validation | Influenza | Influenza virus | whole |
| | (robustness) | virus vs. | | blood |
| | | healthy | | |
| GSE111368 | Validation | Influenza | Influenza virus | whole |
| | (robustness) | virus vs. | | blood |
| | | healthy | | |
| GSE40012 | Validation | Influenza | Influenza virus | whole |
| | (robustness) | virus vs. | | blood |
| | | healthy | | |
| GSE6269 | Validation | Influenza | Influenza virus | PBMCs |
| | (robustness) | virus vs. | | |
| | | healthy | | |
| GSE42026 | Validation | Non- | Respiratory syncytial virus | whole |
| | (cross- | influenza | | blood |
| | reactivity) | virus vs. | | |
| | | healthy | | |
| GSE68310 | Validation | Non- | Coronavirus (seasonal); | PBMCs |
| | (cross- | influenza | Enterovirus; | |
| | reactivity) | virus vs. | Respiratory syncytial virus; | |
| | | healthy | Rhinovirus | |
| GSE29385 | Validation | Non- | Rhinovirus | whole |
| | (cross- | influenza | | blood |
| | reactivity) | virus vs. | | |
| | | healthy | | |
| GSE67059 | Validation | Non- | Rhinovirus | whole |
| | (cross- | influenza | | blood |
| | reactivity) | virus vs. | | |
| | | healthy | | |
| GSE67059 | Validation | Non- | Rhinovirus | whole |
| | (cross- | influenza | | blood |
| | reactivity) | virus vs. | | |
| | | healthy | | |
| GSE103842 | Validation | Non- | Respiratory syncytial virus | whole |
| | (cross- | influenza | | blood |
| | reactivity) | virus vs. | | |
| | | healthy | | |
| GSE34205 | Validation | Non- | Respiratory syncytial virus | PBMCs |
| | (cross- | influenza | | |
| | reactivity) | virus vs. | | |
| | | healthy | | |
| GSE97741 | Validation | Non- | Respiratory syncytial virus; | whole |
| | (cross- | influenza | Rhinovirus | blood |
| | reactivity) | virus vs. | | |
| | | healthy | | |
| GSE103119 | Validation | Non- | Adenovirus; Bocavirus; | whole |
| | (cross- | influenza | Coronavirus; | blood |
| | reactivity) | virus vs. | Human metapneumovirus; | |
| | | healthy | Human parainfluenza virus; | |
| | | | Respiratory syncytial virus; | |
| | | | Rhinovirus | |
| GSE38900 | Validation | Non- | Respiratory syncytial virus | whole |
| | (cross- | influenza | | blood |
| | reactivity) | virus vs. | | |
| | | healthy | | |
| GSE38900 | Validation | Non- | Respiratory syncytial virus | whole |
| | (cross- | influenza | | blood |
| | reactivity) | virus vs. | | |
| | | healthy | | |

TABLE 3.6

| Symbol | Entrez ID | Direction |
|---|---|---|
| MT1IP | 644314 | + |
| CARD17 | 440068 | + |
| H2AC21 | 317772 | + |
| FRMD3 | 257019 | + |
| SAMD9L | 219285 | + |
| NCOA7 | 135112 | + |
| GALM | 130589 | + |
| CMPK2 | 129607 | + |
| ANKRD22 | 118932 | + |
| TRIM6 | 117854 | + |
| BATF2 | 116071 | + |
| EPSTI1 | 94240 | + |
| NEXN | 91624 | + |
| RSAD2 | 91543 | + |
| DDX60L | 91351 | + |
| HELZ2 | 85441 | + |
| TRIM5 | 85363 | + |
| NTNG2 | 84628 | + |
| PARP9 | 83666 | + |
| ZBP1 | 81030 | + |
| DHX58 | 79132 | + |
| KCTD14 | 65987 | + |
| CERK | 64781 | - |
| IFIH1 | 64135 | + |
| RTP4 | 64108 | + |
| SQOR | 58472 | + |
| H2AJ | 55766 | + |
| DDX60 | 55601 | + |
| AKIRIN2 | 55122 | + |
| HERC6 | 55008 | + |
| SAMD9 | 54809 | + |
| XAF1 | 54739 | + |
| GPR84 | 53831 | + |
| ETV7 | 51513 | + |
| EIF3L | 51386 | - |
| MS4A4A | 51338 | + |
| PLAC8 | 51316 | + |
| NT5C3A | 51251 | + |
| SHISA5 | 51246 | + |
| HERC5 | 51191 | + |
| LAP3 | 51056 | + |
| NOP53 | 29997 | - |
| TOR1B | 27348 | + |
| TIMM10 | 26519 | + |
| FBX06 | 26270 | + |
| SPATS2L | 26010 | + |
| IFIT5 | 24138 | + |
| ACOT9 | 23597 | + |
| TDRD7 | 23424 | + |
| RGL1 | 23179 | + |
| USP18 | 11274 | + |
| IFI44L | 10964 | + |
| MTHFD2 | 10797 | + |
| TNFSF13B | 10673 | + |
| MXD4 | 10608 | - |
| DRAP1 | 10589 | + |
| IFI44 | 10561 | + |
| IFITM3 | 10410 | + |
| TRIM22 | 10346 | + |
| LHFPL2 | 10184 | + |
| DHRS9 | 10170 | + |
| SCO2 | 9997 | + |
| ISG15 | 9636 | + |
| AIM2 | 9447 | + |
| OTOF | 9381 | + |
| PSTPIP2 | 9050 | + |
| TNFSF10 | 8743 | + |
| EIF3F | 8665 | - |
| OASL | 8638 | + |
| IFITM1 | 8519 | + |
| H4C8 | 8365 | + |
| H2AC20 | 8338 | + |
| H2AC18 | 8337 | + |
| TNFAIP6 | 7130 | + |
| DYNLT1 | 6993 | + |
| TCN2 | 6948 | + |
| TAP1 | 6890 | + |
| SIGLEC1 | 6614 | + |
| SAT1 | 6303 | + |
| RPS3 | 6188 | - |
| RPL4 | 6124 | - |
| RNASE2 | 6036 | + |
| QARS1 | 5859 | - |
| EIF2AK2 | 5610 | + |
| SEPTIN4 | 5414 | + |
| PLSCR1 | 5359 | + |
| OAS3 | 4940 | + |
| OAS2 | 4939 | + |
| OAS1 | 4938 | + |
| GADD45B | 4616 | + |
| MX1 | 4599 | + |
| MT1G | 4495 | + |
| MT1A | 4489 | + |
| MEF2D | 4209 | - |
| LY6E | 4061 | + |
| LMNB1 | 4001 | + |
| LGALS3BP | 3959 | + |
| IRF7 | 3665 | + |
| IL 1RN | 3557 | + |
| IFIT3 | 3437 | + |
| IFIT1 | 3434 | + |
| IFI35 | 3430 | + |
| IFI27 | 3429 | + |
| IFI16 | 3428 | + |
| ID3 | 3399 | - |
| MR1 | 3140 | + |
| H2BC5 | 3017 | + |
| GPD2 | 2820 | + |
| GNG5 | 2787 | + |
| GBP1 | 2633 | + |
| IFI6 | 2537 | + |
| FCGRIB | 2210 | + |
| FBL | 2091 | - |
| EIF4B | 1975 | - |
| EEF2 | 1938 | - |
| EEF1G | 1937 | - |
| SCARB2 | 950 | + |
| CASP1 | 834 | + |
| VPS51 | 738 | - |
| C3AR1 | 719 | + |
| C1QB | 713 | + |
| SERPING1 | 710 | + |
| CEACAMI | 634 | + |
| ABCA1 | 19 | + |

To characterize the performance space of signatures derived using these candidate genes, the present disclosure generated a set of 100,000 synthetic signatures through random sampling (see Methods). For each generated signature the present disclosure assessed robustness using four independent influenza infection datasets and cross-reactivity using 12 datasets profiling other non-influenza respiratory viruses (FIG. 35D, data accessions in Table 3.5). While most synthetic signatures were robust, they were also cross-reactive (FIG. 35D), likely reflecting shared biology between respiratory virus infection responses.

Previous work has suggested that inclusion of infectious controls may reduce signature cross-reactivity (Sampson et al., 2017; Sweeney et al., 2016; and Tsalik et al., 2016). Following this approach, a meta-analysis of four datasets profiling both influenza and non-influenza viral infections was performed, and 179 candidate genes that were differentially expressed between these two groups (Table 3.6, see Methods) were identified. A set of 100,000 synthetic signatures was generated by randomly sampling from these genes. In this case, the signatures spanned a much wider range of robustness and cross-reactivity values, and identify a subset of signatures that were robust without being cross-reactive were identified (FIG. 35E). These results showed that single-pathogen signatures can satisfy both objectives through the inclusion of targeted infections as control groups.

Examining the performance of all signatures in FIG. 35E, it was found that robustness and cross-reactivity were positively correlated (r=0.69). This suggested that maximizing robustness and minimizing cross-reactivity are conflicting objectives. A salient way to study such a trade-off is to analyze the Pareto efficient solutions, a concept developed in multi-objective optimization (Emmerich and Deutz, 2018). In the general case, Pareto efficient solutions to a multi-objective optimization problem are the ones for which no individual objective (e.g., robustness) can be improved without impairing the other objective (e.g., cross-reactivity). Over the space of candidate signatures, the set of Pareto efficient solutions, called the Pareto front, corresponds to signatures with locally optimal robustness and cross-reactivity characteristics (FIGS. 35D-35E, white points).

To explore factors that may influence the tradeoff between robustness and cross-reactivity of influenza signatures, the present disclosure analyzed the properties of signatures along the Pareto front (FIG. 35E). To increase the number of observations for this analysis, the present disclosure augmented the Pareto front with signatures in a proximal neighborhood, for a total of 100 signatures. (FIG. 35E gray points, see Methods). Looking along the augmented Pareto front, the present disclosure found that the signature size was positively correlated with robustness (r=0.50, FIG. 35F). However, signatures with larger size also suffered from higher cross-reactivity (r=0.52). See FIG. 35H. Furthermore, the present disclosure found that positive and negative genes in the signatures played different roles. After removing negative genes, signatures composed only of positive genes had better robustness but higher cross-reactivity. Conversely, after removing positive genes, signatures composed only of negative genes had worse robustness but reduced cross-reactivity (FIG. 35G). These results combined suggest that signature size and inclusion of both positive and negative genes are critical factors in designing optimal single-pathogen signatures.

### a Web Application to Assess Signature Performance

To make our dataset curations available to the wider research community, the present disclosure has created a web application that allows users to upload gene signatures and evaluate their performance in viral infection, bacterial infection, parasitic infection, aging, and obesity datasets. This tool is available at kleinsteinlab. shinyapps.io/compendium_shiny_app/.

### 3.4. Discussion

In this study, the present disclosure established a framework for benchmarking the performance of host transcriptional response signatures of infection. The scope of our study was fundamentally different from the scope of previous studies that focused on deriving potential signatures of viral or bacterial infections. Going beyond initial efforts to compare the robustness of existing signatures (Bodkin et al., 2022; Tsalik et al., 2016; Warsinske et al., 2019), the evaluation framework of the present disclosure is the first to provide a reference space where any arbitrary signature of infection can be rigorously assessed along two equally critical axes, robustness and cross-reactivity. The framework is based on an extensive data curation of 17,105 blood transcriptional profiles from infectious and non-infectious conditions combined with a universal, model-free signature scoring method. By evaluating the robustness and cross-reactivity of 30 published and 200,000 synthetic signatures, the present disclosure gained new insight towards the implementation of host response assays for clinical infection diagnosis.

In some embodiments, the systems and methods of the present disclosure provide an evaluation that found that most signatures were remarkably robust in detecting their intended conditions, consistent with previous work (Bodkin et al., 2022). Signatures generalized well to independent cohorts, and signatures intended to broadly detect viral or bacterial infection even generalized to pathogens not included in their discovery data. Signatures were also robust to varying infection severity and clinical phase, albeit with reduced performance. Viral signatures also remained robust for several days post-infection, suggesting signatures are capturing sustained biological processes. These findings raise the question as to what biological underpinnings make the signatures of infection so robust. In the case of viral infections, the present disclosure observed that all robust signatures included members of the type-I IFN pathway, a highly conserved antiviral mechanism. Generally, signatures of infection may be more robust if they capture immunological pathways conserved across a pathogen class. Consistent with this hypothesis, it was envisaged that signatures of infection that explicitly include relevant immunological pathways would provide further gains in robustness.

Additional curation and analysis are required to verify that the results are consistent for RNA-seq data, as all compendium data and signatures, with the exception of B7, were derived from microarray platforms. Given the enhanced sensitivity of RNA-seq measurements over those from microarrays, it is expected that the disclosed framework would systematically underestimate the performance of signature B7. Despite this, B7 was found to be robust, even in bacterial pathogens for which it was not explicitly designed (FIGS. 34B and 34E). B7 does not contain negative genes, and therefore it is not expected that improved detection would suppress the cross-reactivity demonstrated in FIG. 35. Technical differences between transcriptional profiling technologies are thus unlikely to change the conclusions of our analysis.

While the evaluated signatures were robust, it was found that they suffered from substantial cross-reactivity in two important ways. First, likely due to significant conservation in immune responses, signatures of infection cross-reacted with unintended pathogen classes (e.g., viral signatures detected bacterial infections, and vice versa). Viral signatures were especially cross-reactive with infections caused by acid-fast bacteria such as Mycobacterium tuberculosis, which may reflect the strong type-I IFN response induced by this pathogen (Berry et al., 2010). This pathogen was the most abundant acid-fast bacterial pathogen, and so it is uncertain whether this is a pathogen-specific effect or if it applies to all bacteria with this cell wall characteristic. Bacterial signatures were slightly more cross-reactive with infections caused by viruses with single-stranded genomes, which suggests conserved immune response mechanisms that require further investigation. Second, both viral and bacterial signatures cross-reacted with aging. This is the first demonstration that signatures of infection can cross-react with non-infectious conditions. From a diagnostic perspective, this in-depth analysis emphasizes the need for infection signatures to undergo extensive cross-reactivity testing before clinical implementation. The cross-reactivity testing should include unintended pathogens, both viral and bacterial, but also aging, and possibly other non-infectious inflammatory conditions.

In depth analysis of published and synthetic influenza specific signatures identified an inherent trade-off between robustness and cross-reactivity. In some embodiments, the systems and methods of the present disclosure also identified several signature properties associated with this trade-off, such as size and the inclusion of both positively and negatively regulated genes. Larger signatures may be more robust but are also less suitable for clinical application: PCR-based diagnostic platforms impose a ceiling on the number of genes that can be measured (Holcomb et al., 2017), and the results of the present disclosure demonstrate that larger signatures are generally more cross-reactive.

Although discovering robust signatures with limited cross-reactivity was beyond the scope of this work, the disclosed results are useful to guide the derivation of future signatures. For example, the disclosed results suggest that including both young and elderly subjects during signature discovery may improve cross-reactivity with aging. Additionally, the present disclosure demonstrates that inclusion of unintended infections as targeted contrasts during signature discovery can greatly reduce cross-reactivity. Because robustness and cross-reactivity are conflicting objectives, pathogen-specific signatures could be identified as solutions of a multi-objective optimization problem, with appropriate constraints on the signature that reflect the desired properties. Developing methods to discover robust signatures which do not cross-react with unintended infections or non-infectious conditions is of great interest.

In summary, the disclosed framework lays the foundation for the discovery of signatures of infection for clinical application. Some embodiments of the present disclosure are implemented as a publicly accessible, user-friendly resource (kleinsteinlab.shinyapps.io/compendium_shiny_app/).

### 3.5. Materials & Methods

### Curation of Infection Signatures

NCBI PubMed searches were performed to identify published signatures of infection using search terms: ‘viral transcriptional signature’, ‘bacterial transcriptional signature’, ‘infection transcriptional signature’, and ‘influenza transcriptional signature”. Inclusion criteria for signatures were that they (1) contain gene lists that describe in-vivo responses to general viral or general bacterial infections in humans; (2) were derived from analyses of PBMCs/whole blood. A separate search for influenza virus infection signatures was performed. The first 200 hits for each search were screened to create a seed pool of papers. The references of these papers, as well as the ‘cited by’ publication results from Google Scholar were screened, for additional signatures that met the inclusion criteria. Signatures published as differentially expressed genes were curated as sets of positive genes (up-regulated in the intended condition) and negative genes (down-regulated in the intended condition). Signatures published as classifiers with coefficients were discretized into positive and negative gene sets based on the sign of the coefficients. The identified signatures were grouped in the following four categories: generic viral (n=11), generic bacterial (n=7), viral versus bacterial (n=6), and influenza-specific (n=6). Enrichment terms were identified using Enrich (Kuleshov et al. 2016).

### Building a Data Compendium

### Dataset Search and Selection

The NCBI GEO was searched for public human expression datasets using an approach modeled after (Sweeney et al., 2016). Infectious exposures were searched in August 2019 with the following keywords: ‘infection’, ‘bact*’, ‘vir*’, ‘fung*’, ‘fever’, ‘sepsis’, ‘pneumonia’, ‘nosocomial’, ‘ICU’, and ‘SIRS’. Non-infectious exposures were searched in January 2020 with keywords ‘age’ and ‘(obesity|BMI)’. For both searches, filters were set to limit results to ‘Homo sapiens’ and ‘high throughput expression profiling by microarray’. For over 8,000 resulting dataset accessions, associated abstracts and included studies that profiled in-vivo infections and non-infectious conditions in PBMCs or whole blood were screened. For infectious exposures the present disclosure included studies that contained at least two conditions and at least 5 samples per condition. These two conditions could be, for example, bacterial and healthy, bacterial and viral, bacterial and non-infectious, bacterial and convalescent, or other permutations of these labels. Studies that compared two pathogens of the same condition type, e.g., comparing one virus against another virus without a non-viral comparison group did not meet this criterion. For non-infectious exposures the present disclosure included studies that profiled the condition of interest and healthy controls. The recount2 database for RNA-seq datasets (Collado-Torres et al., 2017) was also searched, but no datasets met the inclusion criteria.

### Dataset Processing

Datasets from studies that met our inclusion criteria were passed through a standardized pre-processing pipeline designed to handle the most common Illumina (GPL10558, GPL6102, GPL6883, GPL6884, GPL6947) and Affymetrix (GPL11532, GPL6244, GPL13158, GPL13667, GPL201, GPL5175, GPL96, GPL97, GPL570, GPL571) platforms. While each accession generally contained a single dataset, a small number of accessions contained multiple independent cohorts that were treated as separate datasets for processing and analysis. Each dataset was passed through a standardized processing pipeline. The pipeline for Illumina platforms utilized the neqc function with background correction from the limma package (v3.42.2) (Ritchie et al., 2015), and the rma function from the affy package (v1.64.0) for Affymetrix arrays (Bolstad et al., 2003). Datasets from Illumina and Affymetrix platforms were quantile normalized. Datasets from other platforms, datasets that did not contain raw data, or datasets with incomplete raw data (e.g., Illumina probe intensities without p-values) were taken in their processed form from the GEO series accession using GEOquery (v2.54.1) (Davis and Meltzer, 2007). Datasets were log 2 transformed where appropriate and shifted to prevent negative expression values. Gene identifiers for all datasets were remapped to ENTREZIDs using AnnotationDbi (v1.52.0) and the latest platform annotation files (Pages et al., 2020). Outlier detection was performed using the ArrayQualityMetrics package (v3.42.0) with default parameters and thresholds (Kauffmann et al., 2009). Briefly, samples were removed if identified as outliers satisfying the following 3 criteria: (1) a large sum of pairwise distances to other samples, (2) a significantly different intensity distribution compared to a pooled distribution from the remainder of the dataset, and (3) a strong trend on an MA plot comparing each sample to a pseudo-sample of dataset median expression values.

### Dataset Annotation

Infection types were manually annotated for each sample using metadata from GEO and methods from each associated publication. Infections were labeled ‘bacterial’, ‘viral’, ‘other non-infectious’, or ‘parasitic’ based on the exposures or pathogens within each dataset. Samples from subjects coinfected with both bacterial and viral pathogens were removed. No fungal infections were identified, despite explicitly including this in our search terms. The causative pathogen was identified for each sample where possible. For longitudinal datasets, subject IDs and time points were collected.

### Sex Imputation

To predict male and female labels for subjects across the compendium, the imputeSex function from the MetaIntegrator package was utilized with default genes, which clusters subjects according to the expression of several genes with sexually dimorphic expression patterns (Haynes et al., 2016).

### Signature Scoring Framework

The evaluation of signature performance was based on the geometric mean scoring (Haynes et al., 2016). This was defined for each sample i as:

$${S}_{i} = {( {\prod }_{g ∈ positive genes} {x}_{i} ( g ) )}^{\frac{1}{{N}_{p}}} - {( {\prod }_{g ∈ negative genes} {x}_{i} ( g ) )}^{\frac{1}{{N}_{n}}}$$

where xᵢ (g) is the expression of gene g in sample i, Nₚ and Nₙ are the number of positive and negative genes in the signature, respectively. The signature score for a sample is the difference between the geometric mean of the expression of the up-regulated genes and the geometric mean of the expression of the down-regulated genes.

For cross-sectional studies, subject scores were determined by the single sample score. For longitudinal studies, subject scores were summarized by taking the maximally discriminative score per subject. The most typical longitudinal design included profiling of multiple time points for the infected group and a single reference time point for the control group. In this case, the subject score for an infected subject is determined by the maximum sample score over time. For designs that included multiple sampling in the control group, the subject score for a control subject is determined by the minimum sample score over time.

Given a signature and a transcriptional contrast, the performance metric is defined as the resulting area under the ROC curve (AUROC). To calculate this metric, subject scores for this contrast were calculated and ranked. The resulting ranking, paired with the binary labels annotating the subjects (e.g., virus-infected or healthy), were then used to compute the study AUROC. AUROCs were computed only for datasets containing 50% or more of both positive and negative signature genes.

### Evaluation of Robustness and Cross-Reactivity

Robustness was evaluated using conditions that match the signature contrast: e.g., evaluating viral signatures in viral datasets. Signatures that generated median AUROCs greater than 0.7 in independent datasets profiling these intended pathogens were considered robust. This threshold roughly corresponds to the AUROC=0.68 using the critical value for a one-sided Mann-Whitney U test where α=0.05, assuming both group sample sizes were equal to 15 (Mason & Graham, 2002). Median sample sizes in our compendium were 75.5 and 63 for datasets profiling viral and bacterial infections respectively, and so these conditions reflect a lower bound above which performance is considered robust.

Cross-reactivity was evaluated using unintended conditions that do not match the signature contrast: e.g., evaluating viral signatures in bacterial datasets. Viral and bacterial signatures that generated median AUROCs greater than 0.6 for profiling unintended conditions were considered cross-reactive. This cross-reactivity threshold was selected as a compromise between (1) absolute lack of signal and (2) an overly stringent cutoff. While a perfectly non-cross-reactive signature would generate an AUROC less than or equal to 0.5, human cohorts can be highly variable and an AUROC slightly above 0.5 does not necessarily indicate biologically meaningful differences between cases and controls. Conversely, an AUROC threshold of 0.7, as used for determining robustness, would reflect an overly stringent condition for determining whether a signature generates signal for an unintended condition. V/B signatures were considered cross-reactive if they generated a median AUROC greater than 0.6 or less than 0.4. This latter condition reflects that the designation of positive and negative genes in V/B signatures is arbitrary (e.g., these signatures could have been recorded with a bacterial versus viral contrast), and therefore prediction in either direction is relevant to cross-reactivity.

### Comparison of Logistic Regression Scoring and Geometric Mean Scoring

The performance of signatures using geometric mean scoring were compared with logistic regression scoring. Logistic regression scoring was only applied to datasets that met specific criteria: (1) cross-sectional study design, (2) measurements for ≥50% of positive and ≥50% of negative signature genes, (3) a greater number of samples than the number of model features, and (4)≥15 cases and >15 controls. Signature V8 was omitted from evaluation because this signature contained many more genes than samples in all datasets.

Logistic regression models were trained using leave-one-out cross-validation with the caret package (v6.0) (Kuhn, 2008). Subject scores were defined as the held-out sample prediction probability. As with geometric mean scoring, these scores were paired with the binary subject labels (e.g., infected or control) to compute the study AUROC. The geometric mean and logistic regression AUROCs were compared using Pearson correlation.

### Clinical Definitions of Aging and Obesity

For datasets profiling aging, healthy subjects over the age of 64 were considered aged. Young controls were healthy individuals under the age of 36. Obesity definitions were taken from the publication associated with each obesity dataset, often but not always corresponding to a BMI greater than or equal to 30.

### Deriving Candidate Influenza Signature Genes

To derive a set of candidate signature genes that distinguished influenza infection from healthy control samples, all datasets in the compendium containing (1) individuals that could be identified as exclusively influenza infected (removing subjects with co-infections) and (2) profiles from healthy control subjects were identified. Datasets were split 70/30 for discovery and validation purposes, and metadata was used to ensure balanced representation of different platform manufacturers, age groups, tissue types, and sample sizes (Table 3.5). For each longitudinal dataset, a single acute time point was selected for analysis. This was the time point closest to hospital admission or median time of peak symptoms for outpatient cohorts. The meta-analysis procedure described in (Haynes et al., 2016) and (Sweeney et al., 2016) was adapted. A leave-one-dataset-out round-robin meta-analysis was performed. Genes with an absolute effect size cutoff≥1.75 and an FDR cutoff<0.01 in all rounds were selected. To account for differences between PBMCs and whole blood, separate meta-analyses were then performed for PBMC and whole blood datasets in the training set. The final selection filtered the first pool of 148 genes to 124 candidate influenza signature genes that showed an absolute effect size≥1.25 in both PBMC and whole blood analyses.

To derive a signature that distinguishes influenza infection from non-influenza viral infections (such as those caused by hRV and RSV), the present disclosure identified all datasets in the compendium profiling subjects infected exclusively with influenza virus as well as subjects infected with non-influenza viruses (Table 3.5). A round-robin meta-analysis was performed as described above with an absolute effect size cutoff≥0.80 and an FDR cutoff≤0.01. The effect size cutoff was adjusted to generate a pool of candidate genes of similar size to the previous analysis. All but one training datasets profiled whole blood, so candidate genes were not further filtered. A total of 179 candidate influenza signature genes were identified. Predictive performance of these genes for discriminating influenza infection from non-influenza infection was validated in held-out datasets (Table 3.5, AUROCs>0.87).

### Sampling Synthetic Influenza Signatures

In some embodiments, the systems and methods of the present disclosure generated 100,000 synthetic signatures from the influenza versus healthy candidate gene pool and an additional 100,000 synthetic signatures from the influenza versus non-influenza virus candidate gene pool, using a common approach. To generate each synthetic signature, a signature size was randomly sampled from a discrete uniform distribution ranging from a minimum of 3 and a maximum corresponding to the pool size minus 3. This range was selected to reduce the number of identical synthetic signatures. A synthetic signature of the selected size was then randomly sampled from the corresponding pool of candidate genes.

### Evaluating Synthetic Signatures

Synthetic signatures were evaluated for robustness in validation datasets profiling influenza infection and healthy controls, as well as for cross-reactivity in datasets profiling non-influenza infection and healthy controls (Table 3.5). For each synthetic signature, an AUROC was computed in each validation dataset. While median AUROCs was reported in other analyses, here a weighted average AUROC (<AUROC>) was reporte. This was done for consistency with the validation procedure of Sweeney et al., 2016, the study that proposed the meta-analysis approach the present disclosure used to derive the initial gene pool. Weights were determined by dataset sample sizes for robustness and cross-reactivity computation.

### Defining the Augmented Pareto Front Set

A local polynomial function was fit to determine the relationship between cross-reactivity and robustness for the set of Pareto front signatures. Residuals from this fitted model were calculated for all synthetic signatures. Signatures were filtered to those with robustness greater than 0.7 and binned into 5 groups with equal robustness bin widths. The signatures corresponding to the 20 smallest residuals per bin were identified. This set of 100 signatures defines the augmented Pareto front, which contains the Pareto front set as well as additional points from its neighborhood.

### Quantification and Statistical Analysis

Analyses were conducted using R. Statistical tests and related details are listed in figure captions.

### Part 4: Systems and Methods for Multi-Objective Optimization Identifies a Specific and Interpretable COVID-19 Host Response Signature

### Description:

The present disclosure addresses the limitations of previous signature discovery approaches by modeling the robustness/cross-reactivity tradeoff with multi-objective optimization.

The instant disclosure provides novel systems and methods for identifying a highly-specific blood-based signature for SARSCoV-2 infection, which was validated in multiple independent cohorts. In some embodiments, robust signatures are more likely to be interpretable because they have captured coherent biological processes. Consistent with this insight, the present methods show that COVID-19 signature is interpretable as a combination of signals from plasmablasts and memory T cells.

In some embodiments, the analysis of single cell transcriptomic data demonstrates that plasmablasts mediate COVID-19 detection and memory T cells control against cross-reactivity with other viral infections.

In some aspects, provided herein is a multi-objective optimization framework that can use both massive public and multi-omics data to identify diagnostic host response signatures. In some embodiments, the signatures developed with this method are robust and specific. The method helps solve the problem of improving the specificity of host response diagnostic tests.

In some embodiments, the present systems and methods provide a multi-objective optimization approach that can use both massive public and multi-omics data to identify a highly robust and not cross-reactive COVID-19 signature.

In some embodiments, the present disclosure provides robust and specific systems and methods that solve the problem of improving the specificity of host response diagnostic tests.

In some embodiments, the optimization framework is based on a multi-objective fitness function that evaluates any proposed signature along with three dimensions: detection, consistency with other data types (e.g., ATAC-seq) and pathway prior data and low cross-reactivity.

One aspect in accordance with part 4 of the present disclosure provides a method for determining whether a subject is infected with SARS-CoV-2. The method comprises obtaining a plurality of discrete attribute values, where each discrete attribute value in the plurality of discrete attribute values represents a transcript abundance of a respective gene in a plurality of genes in a biological sample from the subject, where the plurality of genes comprises three or more genes in the group consisting of PIF1, BANF1, ROCK2, DOCK5, SLK, TVP23B, GUDC1, ARAP2, SLC25A46, TCEAL3, and EHD3. The method further comprises inputting the plurality of discrete attribute values into a model comprising a plurality of parameters, wherein the model applies the plurality of parameters to the plurality of discrete attribute values to generate as output from the model an indication as to whether the subject is infected with SARS-CoV-2.

In some embodiments, the biological sample is a blood sample comprising plasmablast cells and T cells.

In some embodiments, the plurality of genes comprises PIF1 and EHD3.

In some embodiments, the plurality of genes comprises PIF1.

In some embodiments, the biological sample is a blood sample comprising at least plasmablast cells.

In some embodiments, each discrete attribute value in in the plurality of discrete attribute values is determined by RNA-sequencing of the biological sample or by ATAC-sequencing of the biological sample.

In some embodiments, the plurality of discrete attribute values is obtained by bulk transcriptome sequencing of nucleic acids in the biological sample.

In some embodiments, the method further comprises obtaining, in electronic form, a plurality of sequence reads from the biological sample, wherein the plurality of sequence reads comprises at least 10,000 RNA sequence reads; and using the plurality of sequence reads to determine each discrete attribute value in the plurality of discrete attribute values. In some such embodiments, the using maps each respective sequence read in the plurality of sequence reads to a reference genome. In some such embodiments, the plurality of sequence reads comprises at least 100,000, at least 1×10⁶, or at least 1×10⁷ sequence reads.

In some embodiments, the biological sample is blood, whole blood, or plasma.

In some embodiments, the biological sample comprises a plurality of mRNA molecules and the obtaining the plurality of sequence reads further comprises sequencing the plurality of mRNA molecules using RNA sequencing.

In some embodiments, the model is selected from the group consisting of: a logistic regression model, a neural network, a support vector machine, a Naive Bayes model, a nearest neighbor model, a boosted trees model, a random forest model, a decision tree, or a clustering model.

In some embodiments, the plurality of parameters comprises 100 or more parameters, 1000 or more parameters, 10,000 or more parameters, 100,000 or more parameters, or 1×10⁶ or more parameters.

In some embodiments, the indication as to whether the subject is infected with SARS-CoV-2 is a likelihood that the subject is infected with SARS-CoV-2.

In some embodiments, the indication as to whether the subject is infected with SARS-CoV-2 is a binary indication as to whether or not the subject is infected with SARS-CoV-2.

In some embodiments, the biological sample comprises serum, urine, cerebrospinal fluid, fecal, saliva, sweat, tears, pleural fluid, pericardial fluid, or peritoneal fluid from the subject.

In some embodiments, the biological sample consists of blood, whole blood, plasma, serum, urine, cerebrospinal fluid, fecal, saliva, sweat, tears, pleural fluid, pericardial fluid, or peritoneal fluid from the subject.

In some embodiments, the plurality of genes comprises four, five, six, seven, eight, nine, or ten or more genes in the group consisting of PIF1, BANF1, ROCK2, DOCK5, SLK, TVP23B, GUDC1, ARAP2, SLC25A46, TCEAL3, and EHD3.

In some embodiments, the plurality of genes consists of four, five, six, seven, eight, nine, or ten or more genes in the group consisting of PIF1, BANF1, ROCK2, DOCK5, SLK, TVP23B, GUDC1, ARAP2, SLC25A46, TCEAL3, and EHD3.

Another aspect in accordance with part 4 of the disclosure provides a computer system for determining whether a subject is infected with SARS-CoV-2. The computer system comprises one or more processors and memory addressable by the one or more processors, the memory storing at least one program for execution by the one or more processors. The at least one program comprises instructions for obtaining, in electronic form, a plurality of discrete attribute values, where each discrete attribute value in the plurality of discrete attribute values represents a transcript abundance of a respective gene in a plurality of genes in a biological sample from the subject, and where the plurality of genes comprises three or more genes in the group consisting of PIF1, BANF1, ROCK2, DOCK5, SLK, TVP23B, GUDC1, ARAP2, SLC25A46, TCEAL3, and EHD3. The at least one program further comprises instructions for inputting the plurality of discrete attribute values into a model comprising a plurality of parameters, where the model applies the plurality of parameters to the plurality of discrete attribute values to generate as output from the model an indication as to whether the subject is infected with SARS-CoV-2.

Another aspect in accordance with part 4 of the disclosure provides anon-transitory computer readable storage medium. The non-transitory computer readable storage medium stores instructions, which when executed by a computer system, cause the computer system to perform a method for determining whether a subject is infected with SARS-CoV-2. The method comprises obtaining, in electronic form, a plurality of discrete attribute values, where each discrete attribute value in the plurality of discrete attribute values represents a transcript abundance of a respective gene in a plurality of genes in a biological sample from the subject, and where the plurality of genes comprises three or more genes in the group consisting of PIF1, BANF1, ROCK2, DOCK5, SLK, TVP23B, GUDC1, ARAP2, SLC25A46, TCEAL3, and EHD3. The method further comprises inputting the plurality of discrete attribute values into a model comprising a plurality of parameters. The model applies the plurality of parameters to the plurality of discrete attribute values to generate as output from the model an indication as to whether the subject is infected with SARS-CoV-2.

### 4.1 Abstract

The identification of a COVID-19 host response signature in blood can increase understanding of SARS-CoV-2 pathogenesis and improve diagnostic tools. Applying a multi-objective optimization framework to both massive public and new multi-omics data, the present disclosure identified a COVID-19 signature regulated at both transcriptional and epigenetic levels. In some embodiments, the systems and methods of the present disclosure validated the signature's robustness in multiple independent COVID-19 cohorts. Using public data from 8630 subjects and 53 conditions, the present disclosure demonstrated no cross-reactivity with other viral and bacterial infections, COVID-19 comorbidities, and confounders. In contrast, all previously reported COVID-19 signatures were associated with significant cross-reactivity. The signature's interpretation, based on cell-type deconvolution and single cell data analysis, revealed prominent yet complementary roles for plasmablasts and memory T cells. While the signal from plasmablasts mediated COVID-19 detection, the signal from memory T cells controlled against cross-reactivity with other viral infections. This framework identified a robust interpretable COVID-19 signature, and is broadly applicable in other disease contexts.

### 4.2 Introduction

The novel coronavirus SARS-CoV-2 and the associated COVID-19 disease have redefined recent history. Compared with other common respiratory illnesses, COVID-19 has a higher incidence of severe disease (Gupta et al., 2020; and Tay et al., 2020), greater need for mechanical ventilation (Phua et al., 2020), and post-acute manifestations (Nalbandian et al., 2021; Su et al., 2022). The molecular basis of these clinical manifestations remains largely unknown. Studies comparing blood transcriptomes of COVID-19 patients and healthy subjects, undertaken to define the host response to SARS-CoV-2, showed complex gene expression changes (Blanco-Melo et al., 2020; Daamen et al., 2021; Xiong et al., 2020). Some of these changes, involving pro-inflammatory cytokines, chemokines, and interferon response genes, share commonalities with other respiratory infections (Aschenbrenner et al., 2021; Lee et al., 2020; and McClain et al., 2021).

In addition to molecular effects shared with other infections, COVID-19 may also induce a specific host response signature, that is, a set of transcriptional alterations not observed in other diseases. The identification of a COVID-19 signature would increase understanding of pathogenesis, and foster new diagnostic tools targeting the host response (Lydon et al., 2019a; Rinchai et al., 2020; Tsalik et al., 2021). Early work to identify a COVID-19 signature compared blood transcriptomes of healthy controls, COVID-19 patients, and patients with other infections, such as influenza, seasonal coronaviruses, and bacterial sepsis (Aschenbrenner et al., 2021; Lee et al., 2020; McClain et al., 2021; Ng et al., 2021; and Thair et al., 2021a). Such studies demonstrated, for the first time, the possibility that COVID-19 transcriptional responses could be distinguished from other common respiratory infections.

These early COVID-19 signature studies had two main limitations. The first limitation involved signature robustness, defined as the ability of a signature to detect a disease state (e.g., COVID-19) consistently in multiple independent cohorts. Due to data scarcity on COVID-19 early in the pandemic, most COVID-19 signatures were developed and tested in the same cohorts and were not validated in other independent cohorts, this being the key and most challenging test of robustness. The second limitation involved signature cross-reactivity, defined as the extent to which a signature is affected by any condition (e.g., influenza) other than the intended one (e.g., COVID-19). In previous works, the cross-reactivity data were restricted to few additional unintended infectious conditions and neglected clinical and epidemiological characteristics often associated with COVID-19. COVID-19 comorbidities (e.g., obesity, hypertension) and risk factors (e.g., age, sex), which pre-exist infection and are identifiable in genomic data, are potential confounders and must be considered to demonstrate lack of signature cross-reactivity (Bhaskaran et al., 2021; and Williamson et al., 2020).

In this aspect of the present disclosure a new multi-objective optimization framework was developed and leveraged an extensive data curation to derive a COVID-19 that is robust, minimally cross-reactive and biologically interpretable. Using this framework, the present disclosure identified an 11-gene COVID-19 signature regulated at the transcriptional and epigenetic level, and validated its ability to detect COVID-19 in multiple independent cohorts. Importantly, the COVID-19 signature exhibited minimal cross-reactivity with infectious and non-infectious conditions, including COVID-19 comorbidities and risk factors. To enable the signature's interpretability, the present disclosure developed a method based on deconvolution of bulk transcriptomes and single-cell RNA-seq data analysis. This analysis suggested that plasmablasts mediated COVID-19 detection, and memory T cells controlled against cross-reactivity with other viral infections.

In some embodiments, the systems and methods of the present disclosure identified a COVID-19 signature, and established an integrative framework that leverages multi-omics data and prior information to identify robust, non cross-reactive and interpretable host response signatures.

### 4.3. Results

### Multi-Objective Framework to Identify a COVID-19 Transcriptional Signature

The strategy for signature discovery had three main objectives: (1) a high disease detection capacity, (2) a low cross-reactivity with other infectious and non-infectious states, and (3) a high degree of interpretability. The primary metric for the first two objectives was the area under the ROC curve (AUROC), when distinguishing samples from two conditions (see Methods). Maximizing the detection capacity has the goal of perfect discrimination in COVID-19 studies, corresponding to AUROC values as close as possible to 1. Conversely, minimizing cross-reactivity corresponds to AUROC values<0.5. While the value of AUROC=0.5 is typically associated with random performance, it is important to note that AUROC values strictly smaller than 0.5 are also consistent with a lack of cross-reactivity (see Methods). To gain interpretability, an approach that explains the signature as a combination of signals from specific immune cell types was developed.

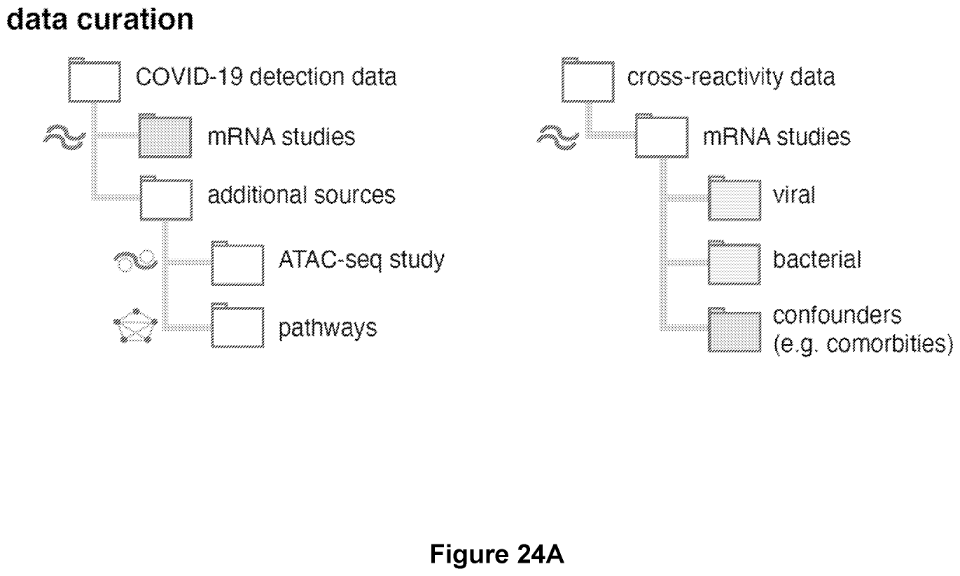

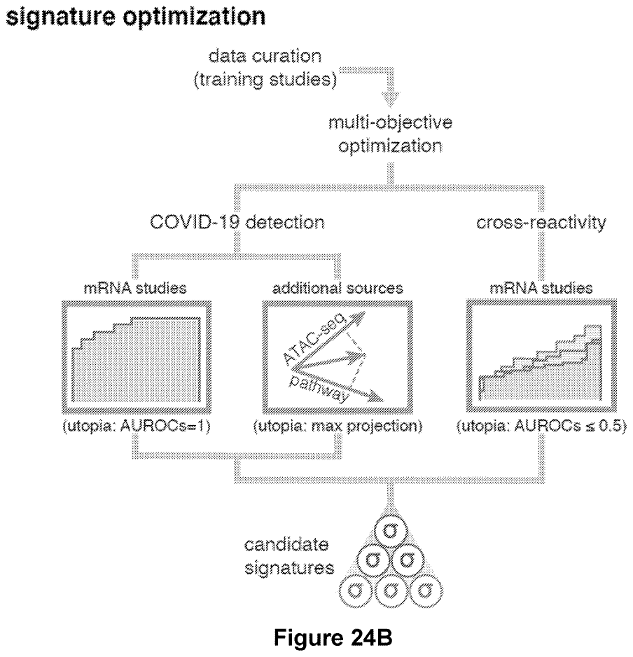

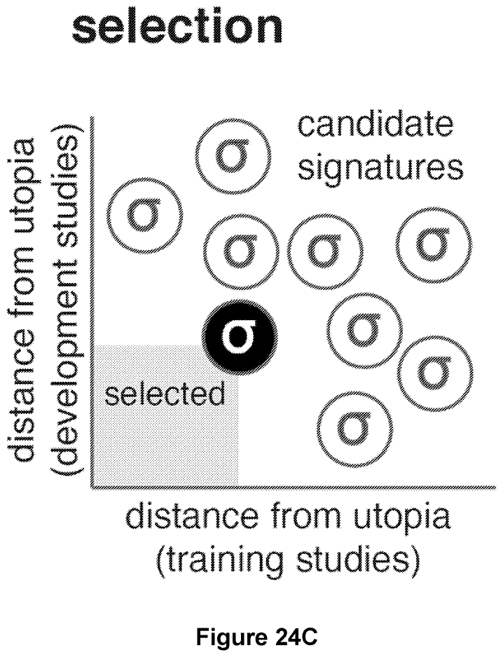

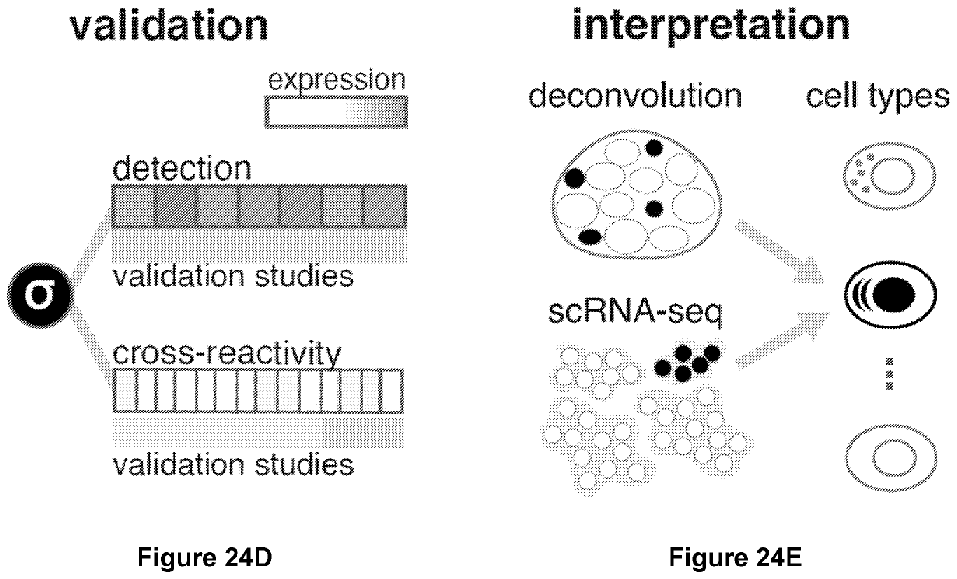

To achieve these objectives, the present disclosure leveraged an existing resource (Chawla et al., 2022) and compiled an extensive data compendium (FIG. 24A, Table 4.1).

TABLE 4.1

| Study | Use | Size | Code | Condition |
|---|---|---|---|---|
| CRA002390 | discov. | 6 | RC | COVID-19 |
| GSE161731 | develop. | 18 | RC | COVID-19 |
| GSE161731 | discov. | 97 | RC | COVID-19 |
| GSE161731 | develop. | 95 | RC | COVID-19 |
| GSE161731 | discov. | 98 | RC | COVID-19 |
| GSE161731 | discov. | 139 | RC | COVID-19 |
| GSE150728 | discov. | 10 | RC | COVID-19 |
| GSE149689 | validat. | 20 | RC | COVID-19 |
| GSE152418 | validat. | 33 | RC | COVID-19 |
| GSE152641 | validat. | 86 | RC | COVID-19 |
| GSE155454 | validat. | 20 | RC | COVID-19 |
| GSE155673 | validat. | 12 | RC | COVID-19 |
| GSE163151 | validat. | 27 | RC | COVID-19 |
| GSE166253 | validat. | 26 | RC | COVID-19 |
| GSE171110 | validat. | 54 | RC | COVID-19 |
| GSE59635 | discov. | 19 | N | Aging |
| GSE59654 | discov. | 22 | N | Aging |
| GSE59743 | discov. | 30 | N | Aging |
| GSE53232 | discov. | 128 | N | Obesity |
| GSE55205 | discov. | 23 | N | Obesity |
| GSE56960 | discov. | 168 | N | Obesity |
| GSE83223 | discov. | 22 | N | Obesity |
| GSE131793 | discov. | 20 | N | Hypertension |
| GSE22356 | discov. | 18 | N | Hypertension |
| GSE42057 | discov. | 135 | N | COPD |
| GSE68526 | discov. | 121 | N | Smoking |
| GSE87072 | discov. | 80 | N | Smoking |
| GSE16129 | develop. | 56 | OB | Staphylococcus aureus |
| GSE62525 | develop. | 28 | OB | Mycobacterium tuberculosis |
| GSE25504 | develop. | 5 | OB | Unknown bacterium |
| GSE69528 | develop. | 111 | OB | Acinetobacter baumannii; Acinetobacter lwoffii; |
| | | | | Aeromonas; Bacillus; Burkholderia |
| | | | | pseudomallei; Citrobacter |
| | | | | freundii; Corynebacterium; |
| | | | | Cryptococcus neoformans; |
| | | | | Enterococcus; Escherichia coli; |
| | | | | Klebsiella; Klebsiella pneumoniae; |
| | | | | Micrococcus; Pseudomonas aeruginosa; |
| | | | | Salmonella; Salmonella group C; |
| | | | | Sphingobacterium; Sphingomonas; |
| | | | | Staphylococcus aureus; |
| | | | | Streptococcus pyogenes; |
| | | | | Streptococcus suis; |
| | | | | Viridans streptococci |
| GSE32707 | develop. | 79 | OB | Unknown bacterium |
| GSE73461 | develop. | 107 | OB | Unknown bacterium |
| GSE103119 | develop. | 73 | OB | Mycoplasma; Streptococcus pneumoniae; |
| | | | | Streptococcus pyogenes |
| GSE13015 | discov. | 34 | OB | Aeromonas hydrophila; |
| | | | | Burkholderia pseudomallei; |
| | | | | Corynebacterium; Enterococcus; |
| | | | | Enterococcusf aecium; Escherichia coli; |
| | | | | Salmonella; Staphylococcus aureus; |
| | | | | Streptococcus Non-Group A or B |
| GSE29161 | discov. | 10 | OB | Unknown bacterium |
| GSE19444 | discov. | 33 | OB | Mycobacterium tuberculosis |
| GSE30385 | discov. | 20 | OB | Unknown bacterium |
| GSE16129 | discov. | 52 | OB | Staphylococcus |
| GSE68004 | discov. | 30 | OB | Streptococcus |
| GSE83456 | discov. | 153 | OB | Mycobacterium tuberculosis |
| GSE60244 | discov. | 62 | OB | Unknown bacterium |
| GSE11755 | discov. | 27 | OB | Neisseria meningitidis |
| GSE40586 | discov. | 39 | OB | Streptococcus pneumoniae; Unknown |
| GSE25504 | discov. | 15 | OB | Unknown bacterium |
| GSE30119 | discov. | 143 | OB | Staphylococcus aureus |
| GSE29536 | discov. | 141 | OB | Burkholderia pseudomallei; Mycobacterium tuberculosis |
| GSE97298 | discov. | 62 | OB | Nontuberculous mycobacteria |
| GSE73464 | discov. | 107 | OB | Unknown bacterium |
| GSE73462 | discov. | 39 | OB | Unknown bacterium |
| GSE13015 | discov | 27 | OB | Acinetobactor baumannii; Burkholderia pseudomallei; |
| | | | | Corynebacterium; Enterococcus; Escherichia coli; |
| | | | | Klebsiella pneumoniae; Salmonella; |
| | | | | Salmonella serotype b; Staphylococcus aureus; |
| | | | | Staphylococcus coagulase negative; |
| | | | | Streptococcus Non-Group A or B; |
| | | | | Streptococcus pneumoniae |
| GSE19439 | discov. | 25 | OB | Mycobacterium tuberculosis |
| GSE41055 | discov. | 18 | OB | Mycobacterium tuberculosis |
| GSE25504 | discov. | 63 | OB | Enterococcus; Escherichia coli; group B |
| | | | | Streptococcus; Staphylococcus |
| GSE73461 | develop. | 149 | OV | Unknown virus |
| GSE73464 | develop. | 149 | OV | Unknown virus |
| GSE29333 | develop. | 18 | OV | Human T-Lymphotropic virus Type 1 |
| GSE74027 | develop. | 99 | OV | Unknown virus |
| GSE57730 | develop. | 12 | OV | Human immunodeficiency virus |
| GSE36539 | develop. | 32 | OV | Hepatitis E virus |
| GSE29536 | develop. | 152 | OV | Human immunodeficiency virus |
| GSE29429 | develop. | 185 | OV | Human immunodeficiency virus |
| GSE29366 | discov. | 31 | OV | Influenza virus |
| GSE13699 | discov. | 22 | OV | Live-attenuated Yellow Fever Virus |
| GSE67059 | discov. | 22 | OV | Rhinovirus |
| GSE4124 | discov. | 45 | OV | Human immunodeficiency virus |
| GSE33580 | discov. | 86 | OV | Human immunodeficiency virus |
| GSE85599 | discov. | 17 | OV | Epstein-Barr Virus |
| GSE6269 | discov. | 24 | OV | Influenza virus |
| GSE60244 | discov. | 111 | OV | Unknown virus |
| GSE103842 | discov. | 74 | OV | Respiratory syncytial virus |
| GSE45918 | discov. | 16 | OV | Epstein-Barr Virus |
| GSE38900 | discov. | 36 | OV | Respiratory syncytial virus |
| GSE29333 | discov. | 19 | OV | Human T-Lymphotropic virus Type 1 |
| GSE103119 | discov. | 115 | OV | Adenovirus; Bocavirus; Coronavirus; Human |
| | | | | metapneumovirus; Human parainfluenza virus; |
| | | | | Respiratory syncytial virus; Rhinovirus |
| GSE29429 | discov. | 47 | OV | Human immunodeficiency virus |
| GSE34205 | discov. | 101 | OV | Influenza virus;Respiratory syncytial virus |
| GSE47199 | discov. | 40 | OV | BK Polyomavirus |
| GSE67059 | discov. | 103 | OV | Rhinovirus |
| GSE2171 | discov. | 34 | OV | Human immunodeficiency virus |
| GSE38900 | discov. | 52 | OV | Influenza virus; Respiratory syncytial virus; Rhinovirus |
| GSE44228 | discov. | 72 | OV | Human immunodeficiency virus |
| GSE20346 | develop. | 44 | RB | Unknown bacterium |
| GSE42825 | discov. | 31 | RB | Mycobacterium tuberculosis |
| GSE42826 | discov. | 63 | RB | Mycobacterium tuberculosis |
| GSE42830 | discov. | 54 | RB | Mycobacterium tuberculosis |
| GSE20346 | develop. | 37 | RV | Influenza virus |
| GSE82050 | discov. | 39 | RV | Influenza virus |
| GSE111368 | discov. | 239 | RV | Influenza virus |
| GSE101702 | discov. | 159 | RV | Influenza virus |
| GSE68310 | discov. | 232 | RV | Coronavirus;Enterovirus; Influenza virus; |
| | | | | Respiratory syncytial virus; Rhinovirus |
| GSE94916 | validat. | 12 | N | COPD |
| GSE101710 | validat. | 20 | N | Aging |
| GSE65219 | validat. | 176 | N | Aging |
| GSE83864 | validat. | 135 | N | Air Pollution |
| GSE69683 | validat. | 333 | N | Asthma |
| GSE54837 | validat. | 142 | N | COPD |
| GSE56766 | validat. | 142 | N | COPD |
| GSE20680 | validat. | 139 | N | Coronary Artery Disease |
| GSE20681 | validat. | 198 | N | Coronary Artery Disease |
| GSE703 | validat. | 20 | N | Hypertension |
| GSE38267 | validat. | 41 | N | Hypertension |
| GSE41233 | validat. | 32 | N | Obesity |
| GSE69039 | validat. | 18 | N | Obesity |
| GSE18897 | validat. | 80 | N | Obesity |
| GSE23323 | validat. | 44 | N | Smoking |
| GSE87005 | validat. | 40 | N | Type 2 Diabetes |
| GSE28623 | validat. | 108 | OB | Mycobacterium tuberculosis |
| GSE36238 | validat. | 18 | OB | Mycobacterium tuberculosis |
| GSE64456 | validat. | 39 | OB | Unknown bacterium |
| GSE64456 | validat. | 69 | OB | Unknown bacterium |
| GSE65682 | validat. | 225 | OB | Unknown bacterium |
| GSE40396 | validat. | 30 | OB | Escherichia coli; Salmonella; |
| | | | | Staphylococcus aureus; Unknown |
| GSE42026 | validat. | 51 | OB | Unknown bacterium |
| GSE117827 | validat. | 50 | OV | Coxsackievirus; Enterovirus; |
| | | | | Respiratory syncytial virus; Rhinovirus |
| GSE40184 | validat. | 18 | OV | Hepatitis C virus |
| GSE40223 | validat. | 10 | OV | Hepatitis C virus |
| GSE40396 | validat. | 57 | OV | Adenovirus; Enterovirus; |
| | | | | Human herpesvirus 6; Rhinovirus |
| GSE42026 | validat. | 74 | OV | Influenza virus;Respiratory syncytial virus |
| GSE51808 | validat. | 56 | OV | Dengue virus |
| GSE59312 | validat. | 79 | OV | Hepatitis C virus |
| GSE81246 | validat. | 48 | OV | Human cytomegalovirus |
| GSE94916 | validat. | 12 | RB | Unknown bacterium |
| GSE40012 | validat. | 79 | RB | Unknown bacterium |
| GSE17156 | validat. | 113 | RV | Influenza virus; Respiratory syncytial virus; Rhinovirus |
| GSE40012 | validat. | 60 | RV | Influenza virus |
| EGAS00001005493 | severity | 99 | RC | COVID-19 |
| E-MTAB-10026 | severity | 113 | RC | COVID-19 |
| EGAS00001004571 | severity | 25 | RC | COVID-19 |

The COVID-19 detection component consisted of human blood transcriptomic studies in the form of COVID-19 vs healthy controls, and COVID-19 vs other pathogens (e.g., influenza or seasonal coronaviruses). To further improve COVID-19 detection, the present disclosure integrated additional data sources: ATAC-seq data for the COVID-19 versus healthy comparison, and gene annotation libraries. The cross-reactivity data (also referred to as ‘non-COVID-19’ data) comprised a set of human blood transcriptional studies classified in three main groups: viral (both respiratory and non-respiratory), bacterial (both respiratory and non-respiratory), and non-infectious. The non-infectious studies included common COVID-19 comorbidities and risk factors such as sex and age, which act as potential confounders.

To build a COVID-19 signature, the present disclosure followed the established machine learning strategy and partitioned the data compendium into training, development (develop.), and validation (validat.) subsets. The validation subset was manually defined to ensure accurate and comprehensive tests of COVID-19 detection and cross-reactivity. With 127 gene expression studies, the curated compendium set the foundation for finding a robust COVID-19 signature that does not cross-react with a broad set of infectious and non-infectious conditions (Table 4.1).

Next, an optimization framework in accordance with the present disclosure that leverages the compendium to discover a COVID-19 transcriptional signature (FIG. 24B) was constructed. In some embodiments, the systems and methods of the present disclosure aimed to identify a compact COVID-19 signature (no more than 12 genes), a small size that is compatible with common PCR diagnostic platforms (Holcomb et al., 2017). The quality of a signature is captured by a multi-objective fitness function aimed to maximize detection and minimize cross-reactivity. The detection fitness objective encompasses discriminative power in COVID-19 gene expression studies and consistency with the additional sources provided by COVID-19 ATAC-seq data and pathway knowledgebase. The consistency with these additional data sources provides independent evidence of the validity of the signature's biological basis. The cross-reactivity fitness objective reflects a lack of discriminative power in non-COVID-19 transcriptomic studies.

The multi-objective fitness function was optimized in the training studies using a genetic algorithm, which returns a population of high-fitness solutions, each corresponding to a candidate signature (see Methods). To select the optimal signature, the generalization performance of each candidate solution was assessed on a set of development studies. The signature showing the most consistent performance in both training and development studies was selected (FIG. 24C). The COVID-19 detection and cross-reactivity of the selected solution was then tested on a third set of independent validation studies (FIG. 24D).

Finally, based on deconvolution of bulk transcriptomes and single-cell RNA-seq data analysis, our framework enables the interpretation of the signature in terms of signals from specific immune cell types (FIG. 24E).

### Identification of an 11-Gene COVID-19 Transcriptional Signature

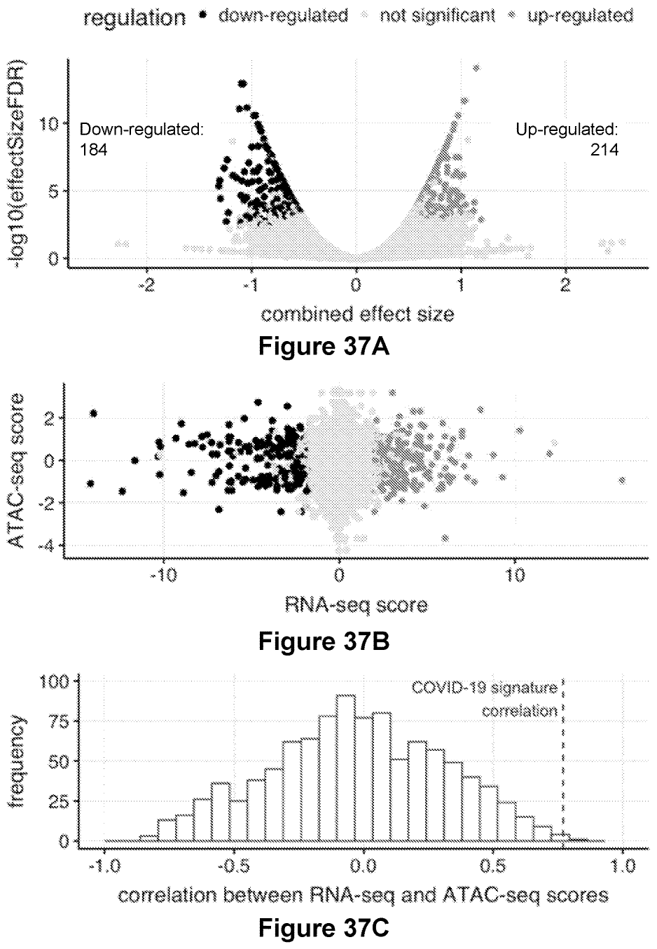

The formulation with multiple, possibly conflicting objectives (e.g., high COVID-19 detection, low cross-reactivity), involved solving a combinatorial optimization problem with a multi-objective fitness function. To reduce the combinatorial space and overall computational time, a meta-analysis of the COVID-19 training studies (Table 4.1) was conducted, pre-selecting a pool of 398 genes as potential members of the COVID-19 signature (see Methods, FIGS. 37A, 37B, 37C, Table 4.2). Additional details and information regarding Table 4.2 is found at Cappuccio et al., “Multi-objective optimization identifies a specific and interpretable COVID-19 host response signature,” Cell Systems 13(12), pg. 989-1001; Supplementary Table 2, which is hereby incorporated by reference in its entirety for all purposes. To generate candidate solutions, the multi-objective optimization problem was converted into a family of scalar subproblems (Emmerich and Deutz, 2018). Each subproblem amounts to maximizing a weighted linear combination of the multi-objective fitness components. The subproblems, each with varying component weights, were solved by applying a genetic algorithm, generating a space of 8305 candidate solutions (see Methods).

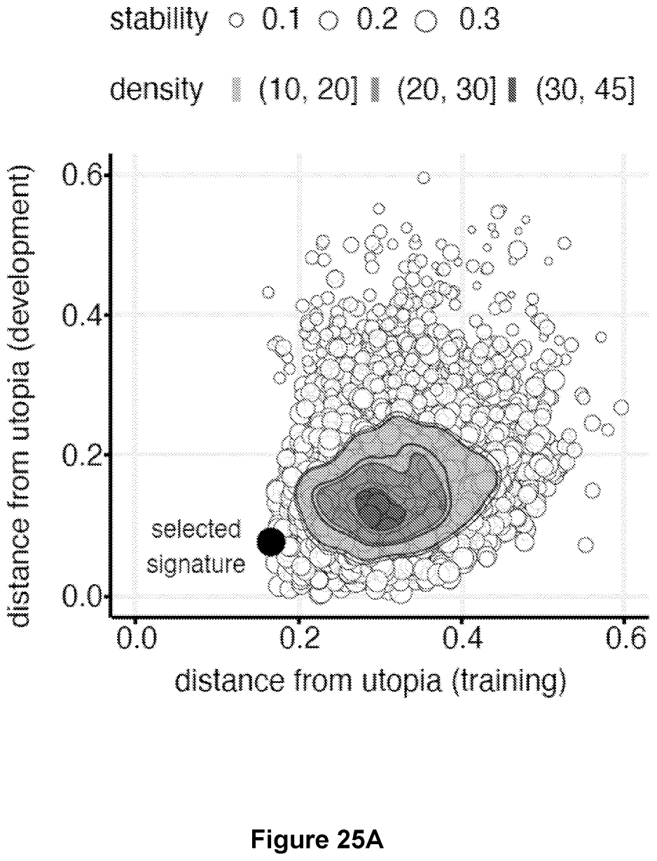

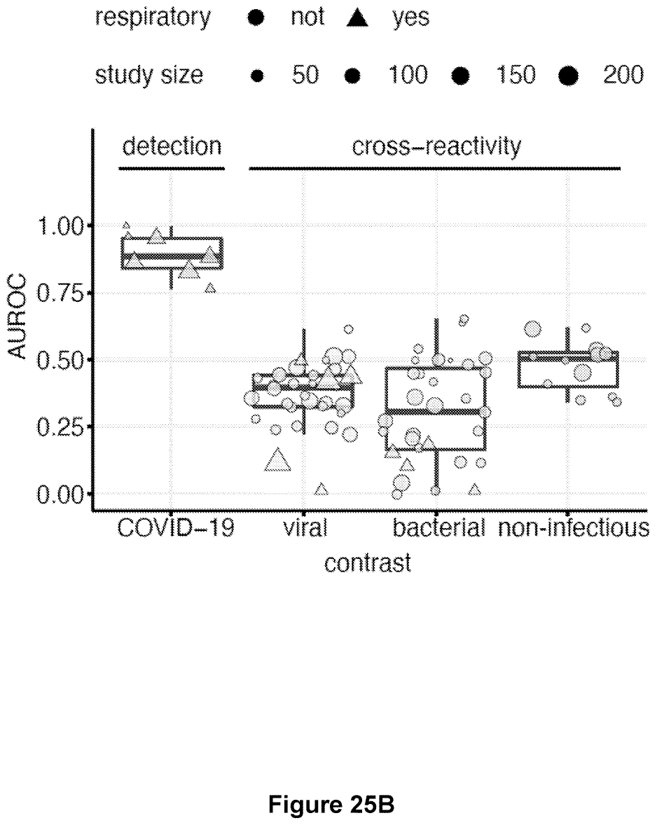

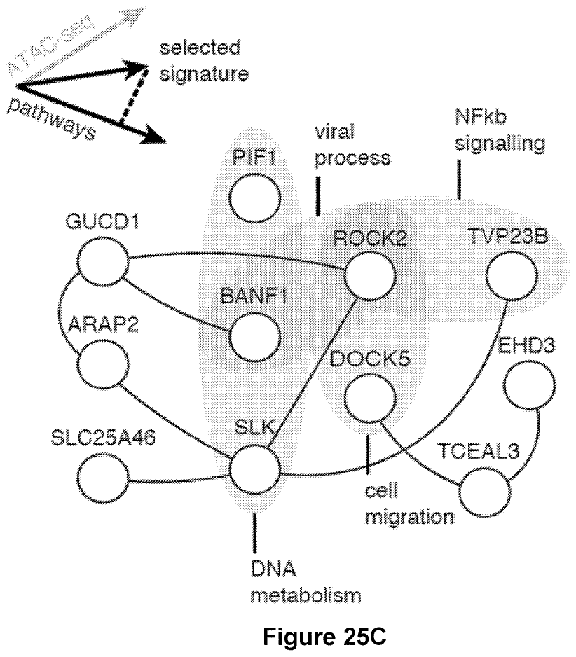

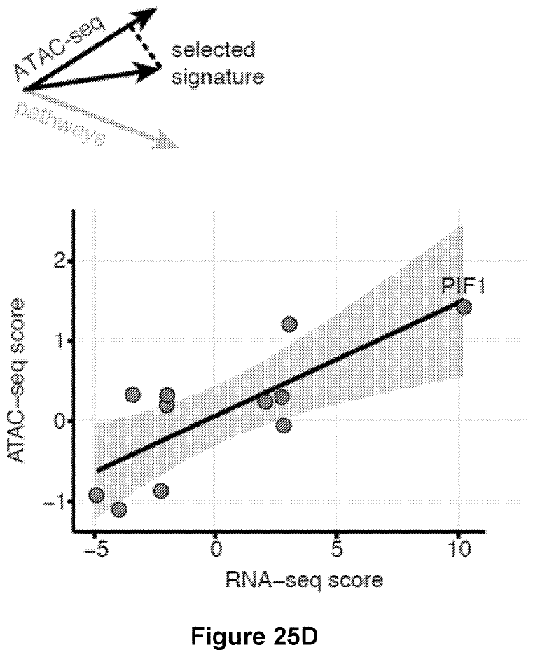

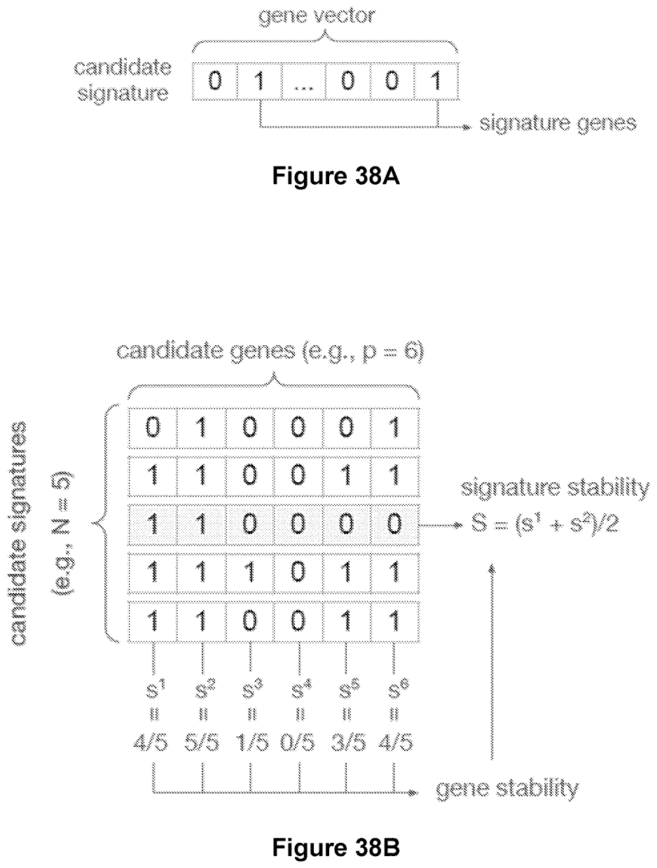

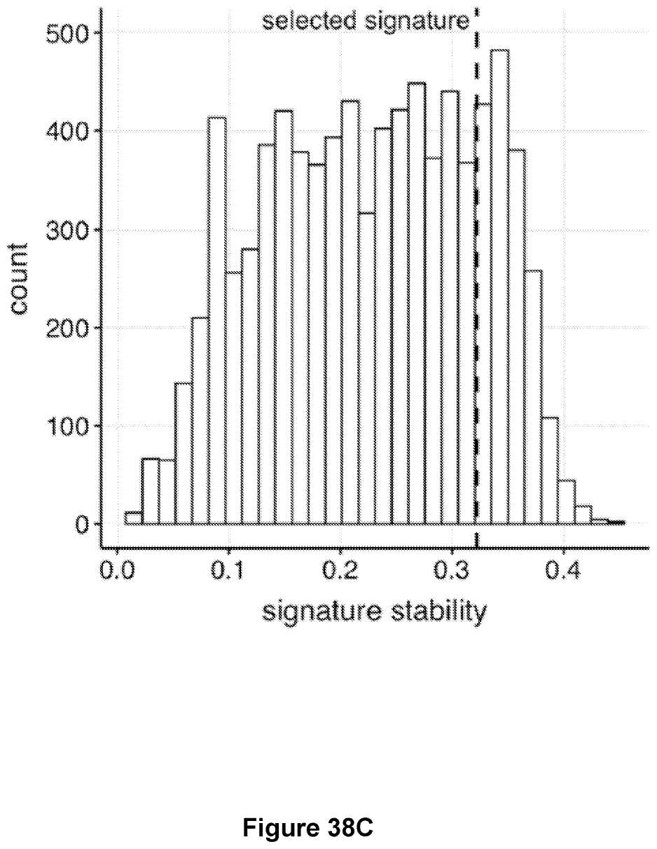

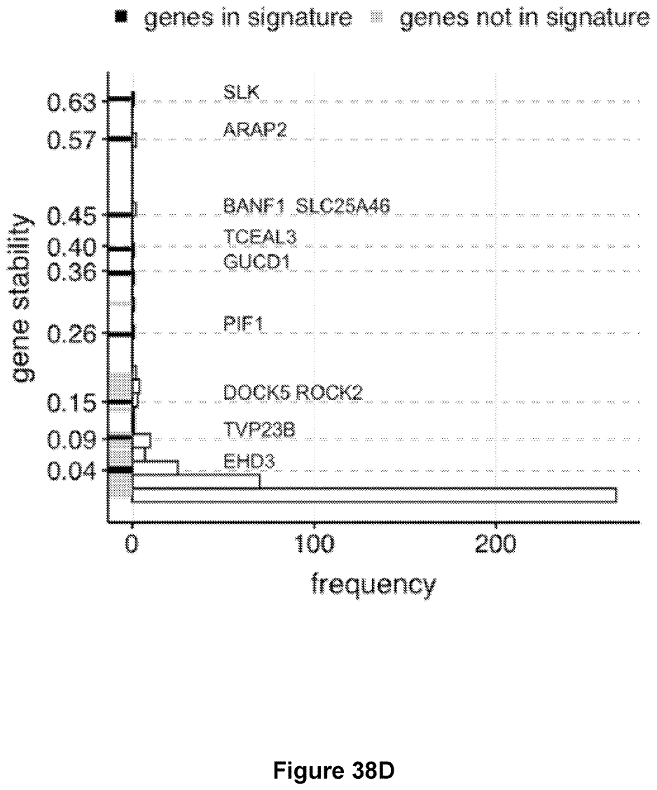

To select an appropriate solution, the present disclosure considered three criteria (FIG. 25A). First, solutions whose performance was as close as possible to the ‘utopia’ signature—one that would result in perfect discrimination in all training COVID-19 studies (AUROC=1.0), and no cross-reactivity in all training non-COVID-19 studies (AUROC<0.5) were prioritized. Second, the present disclosure prioritized solutions whose performance was consistently close to the utopia point in a separate set of development studies, not used for training. The latter criterion, typical of machine learning, aimed to control over-fitting to the training studies, and to increase generalizability of the results to other independent studies. Third, solutions whose genes appeared more frequently in the overall solution space were priortized, to ensure a more robust selection process (see Methods, FIGS. 38A, 38B, 38C, and 38D).

By applying the above criteria, the present disclosure selected a signature of eleven genes (FIG. 25A). In training and development studies, the selected signature showed a consistently high detection in COVID-19 contrasts (median AUROC=0.89; IQR: 0.84-0.95). These included four studies comparing COVID-19 vs healthy controls, and three studies directly comparing COVID-19 vs other pathogens. The signature achieved low cross-reactivity with respect to viral (median AUROC=0.39; IQR: 0.32-0.44), bacterial (median AUROC=0.30; IQR: 0.16-0.46), and non-infectious contrasts (median AUROC=0.51; IQR: 0.43-0.52) (FIG. 25B).

In some embodiments, the systems and methods of the present disclosure next conducted a stability analysis to investigate to what extent the genes identified in the final selected signature tended to be characteristic of the overall “near optimal” solution space. In some embodiments, the systems and methods of the present disclosure found that while four genes were relatively rare (4%-17%), seven of the eleven signature genes appeared frequently in the overall solution space (40%-69%), confirming a predominantly stable solution (FIGS. 38A, 38B, 38C, and 38D). The consistency of the selected signature was then analyzed with respect to the additional data sources, gene annotation libraries and ATAC-seq data. Network analysis (Greene et al., 2015) revealed functional, blood-specific connections among the signature genes (FIG. 25C). Despite its limited size, the signature captured gene annotations consistent with host response to infection, including ‘viral process’, ‘NF-kb signaling’, and ‘cell migration’. Furthermore, the transcriptional and epigenetic regulation of the signature genes by COVID-19 were highly correlated (Pearson correlation=0.77), with PIF1 displaying the strongest up-regulation in both data types (FIG. 25D). The observed correlation level between the transcriptional and the epigenetic regulation in the selected COVID-19 signature was significantly higher than what was expected from a signature of the same size randomly extracted from the pool of pre-selected genes (p=0.001, see Methods, FIGS. 37A, 37B, and 37C). This indicated that the selection process favored genes with a consistent transcriptional and epigenetic regulation.

Overall, the optimization framework produced a signature able to detect COVID-19 with minimal cross-reactivity, and showed consistency with immune pathways and with epigenetic data.

### Multi-Cohort Validation of the COVID-19 Signature

Next, the present disclosure assessed the generalization performance of the signature with a multi-cohort validation involving studies not used in the signature discovery (discov.) and development (develop.). Eight additional COVID-19 studies were retrieved from the public domain, which included bulk RNA-seq data and pseudo-bulk RNA-seq data generated from single cell studies from both PBMC and whole blood. The COVID-19 validation studies were retrieved and processed after the signature development, to avoid potential data leakage.

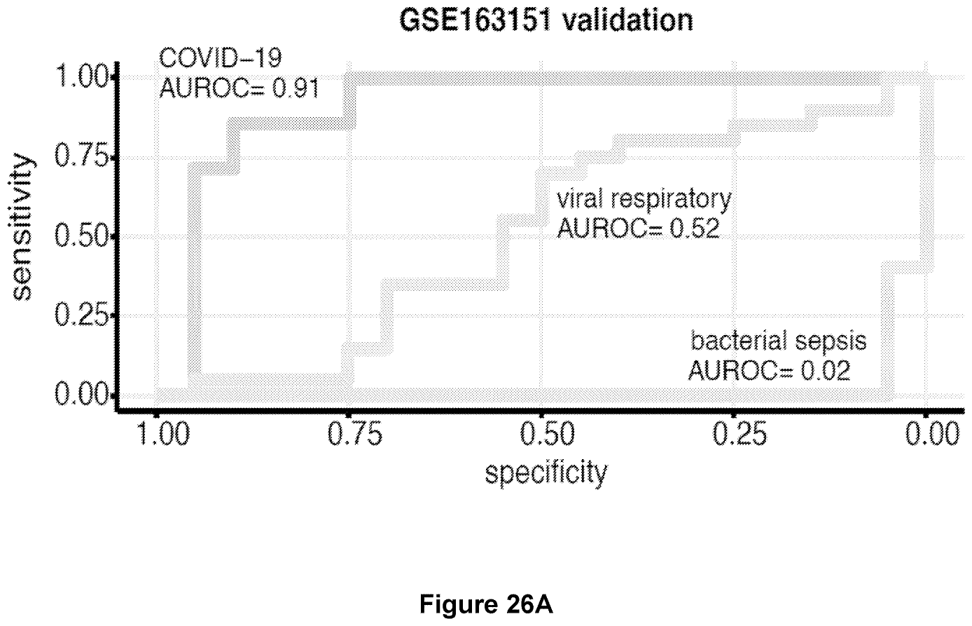

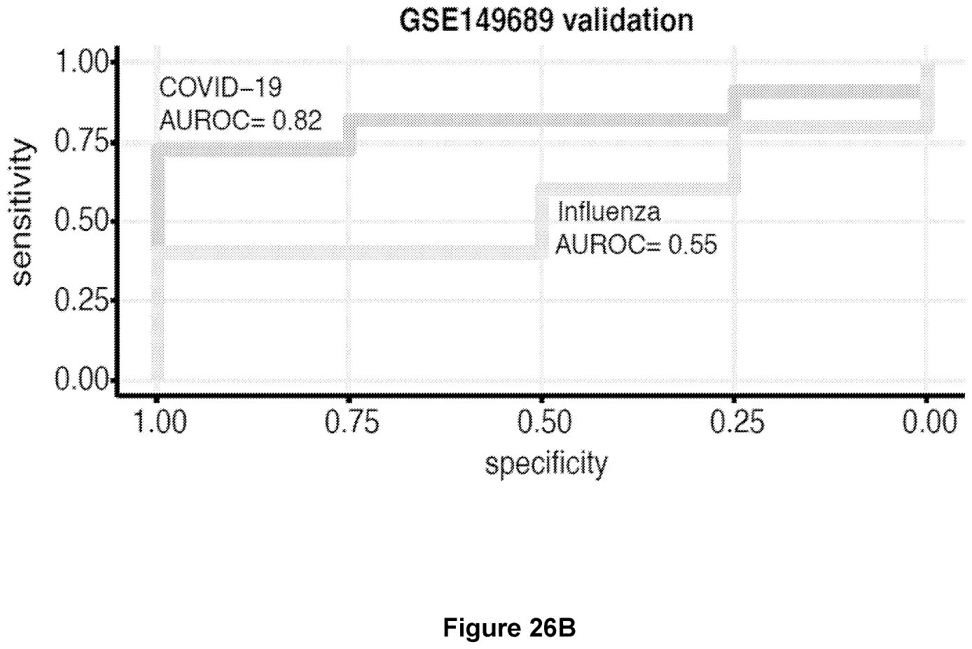

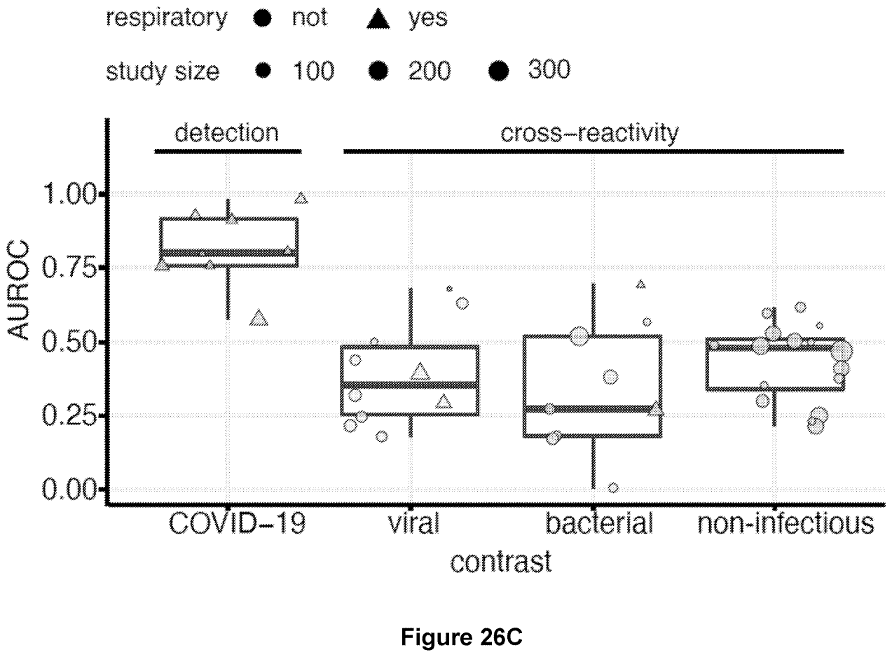

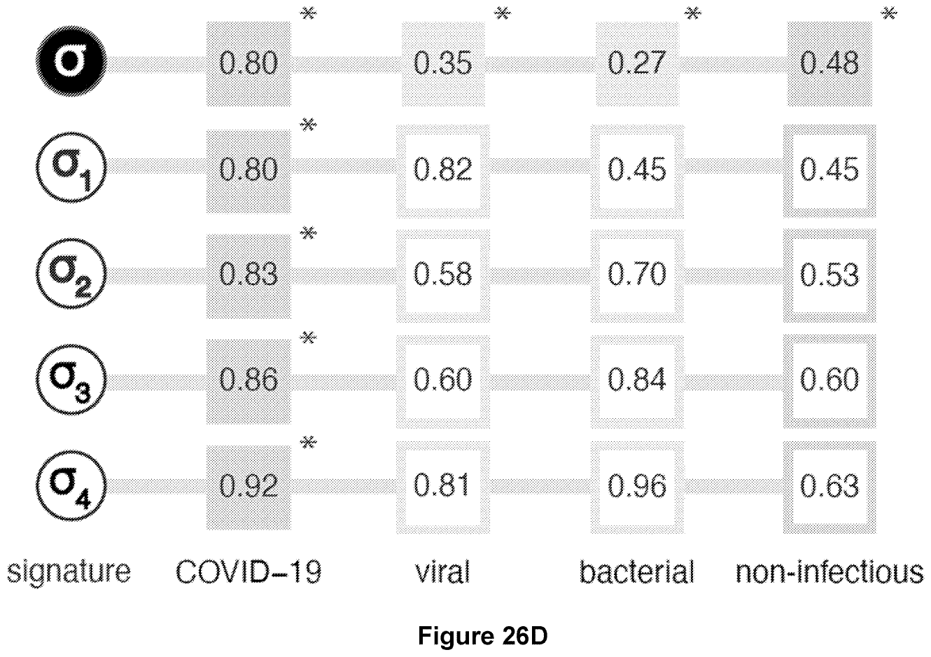

Two of the available studies (GSE163151 and GSE149689) contained blood transcriptomes from subjects in multiple conditions, including COVID-19, viral acute respiratory illness, bacterial sepsis, and healthy controls. Thus, these studies provided the opportunity to test the COVID-19 detection along with the cross-reactivity with viral and bacterial infections. In both studies, the COVID-19 signature had a high COVID-19 detection rate (AUROC=0.91 in GSE163151, and AUROC=0.82 in GSE149689), and minimal cross-reactivity with other infections (AUROCS0.55) (FIGS. 26A and 26B). Except for one study (GSE152641, AUROC=0.57), the signature performance in the full set of COVID-19 validation studies was consistent with the training and development studies, indicating effective generalizability (median AUROC=0.80, IQR: 0.76-0.92) (FIG. 26C).

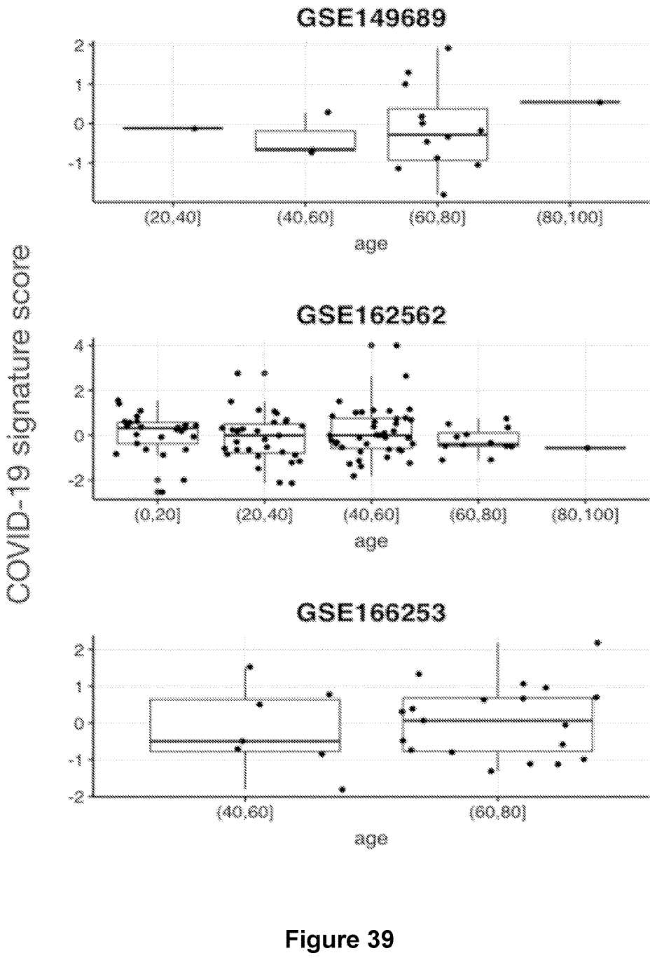

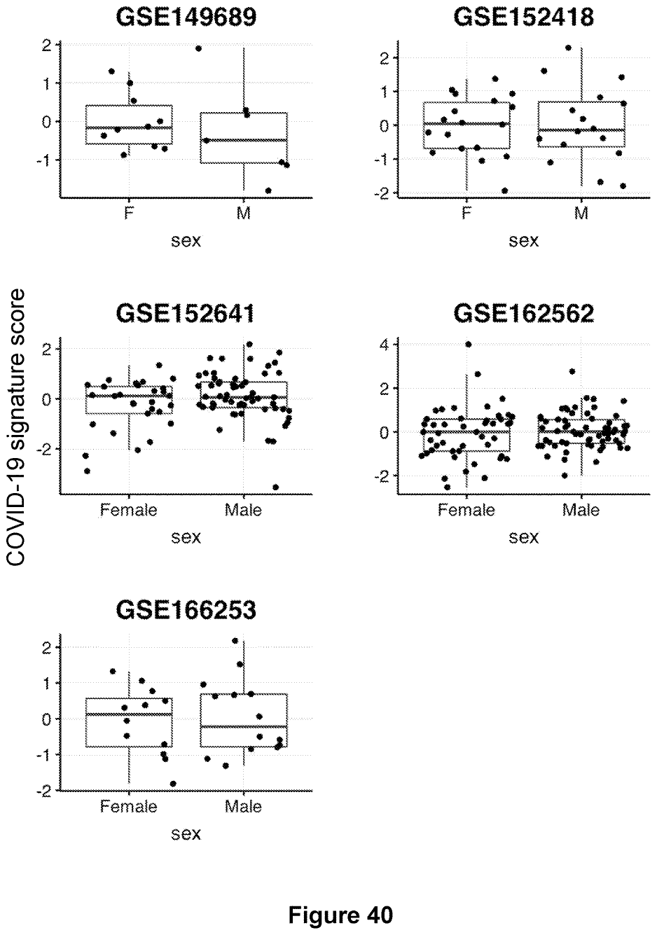

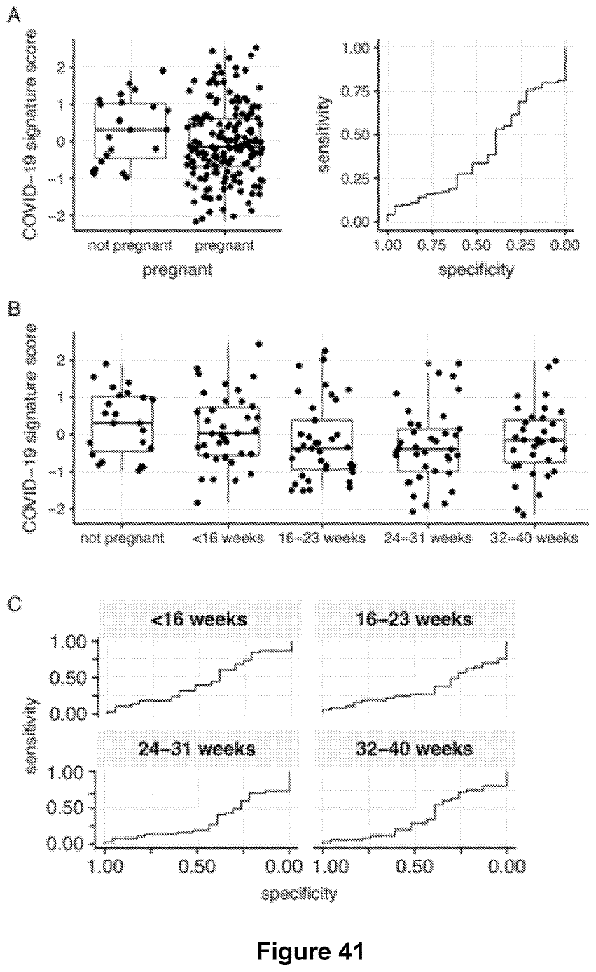

The COVID-19 signature cross-reactivity was tested in validation studies profiling a broad variety of infectious and non-infectious conditions. The infectious contrasts included transcriptional profiles of subjects with common respiratory viral illnesses, for example, caused by the influenza virus, respiratory syncytial virus, and human rhinovirus, as well as subjects with bacterial pneumonia. Non-infectious conditions included age, sex and COVID-19 comorbidities, such as COPD, obesity and hypertension. Consistent with the training and development studies, the resulting AUROC distributions for all conditions tested (FIGS. 26C, 39, and 40) resembled the performance of a random classifier, supporting marginal cross-reactivity with all the study classes. In particular, the signature did not cross-react with COPD (median AUROC of three studies: 0.50), obesity (median AUROC of three studies: 0.49), and hypertension (median AUROC of two studies: 0.30), which are common COVID-19 comorbidities. In some embodiments, the systems and methods of the present disclosure additionally tested whether the COVID-19 signature showed cross-reactivity in healthy women during pregnancy, and found no evidence of cross-reactivity throughout the entire pregnancy time-course (AUROC<0.44, FIG. 41).

Finally, the overall performance of the COVID-19 signature was compared with the performance of four previously published signatures: σ1 (Thair et al., 2021a), σ2 (Lee et al., 2020), σ3 (McClain et al., 2021), and σ4 (Aschenbrenner et al., 2021). These signatures were derived from data on COVID-19 patients and healthy controls, while also including samples with infectious conditions intended to mitigate signature cross-reactivity. To assess the performance of a signature, two metrics in the same set of validation studies were evaluated: (1) the median AUROC values across studies in each of the four classes; (2) a significance p-value based on hypothesis testing, applying a conventional threshold of p<0.05 for significance. In the case of COVID-19 detection, this was tested against the null hypothesis of no COVID-19 detection, which corresponds to an AUROC distribution with mean<0.5. In the case of cross-reactivity, this was tested against the null hypothesis stating the presence of cross-reactivity, which corresponds to an AUROC distribution with mean≥0.5.

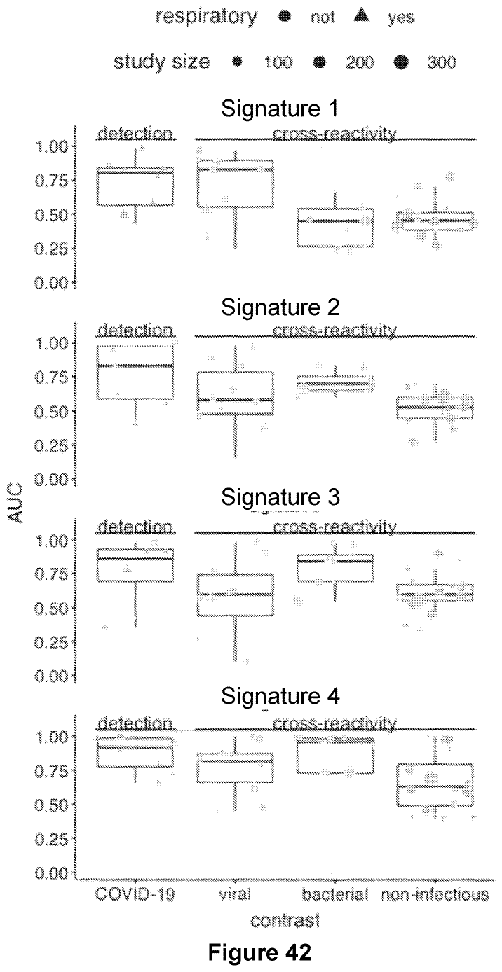

All signatures provided robust detection across multiple COVID-19 studies, with median AUROC values greater or equal to 0.8 and significant p-values (FIG. 26D, FIG. 42). However, only the COVID-19 signature developed here showed no cross-reactivity with viral, bacterial, and non-infectious conditions, achieving median AUROC values less than 0.5 and significant p-values in the three categories (FIG. 26D, FIG. 42). For all the other signatures, while some median AUROC values were below 0.5, the AUROC distributions showed large deviations, and the null hypothesis stating the presence of cross-reactivity could not be rejected. While cross-reactivity with other viral infections is expected, more surprising was the presence of cross-reactivity with bacterial and non-infectious conditions. In particular, σ₄ largely cross-reacted with studies on COVID-19 risk factors, such as COPD (AUROC=1.0), hypertension (AUROC=0.98), and aging (AUROC=0.97).

Altogether, the disclosed signature's performance in validation studies was highly concordant with the results in training and development sets, generalizing well in both COVID-19 and non-COVID-19 validation studies. Furthermore, the COVID-19 signature identified with our approach outperformed all previously published COVID-19 signatures.

COVID-19 Signature Performance Increases with Disease Severity

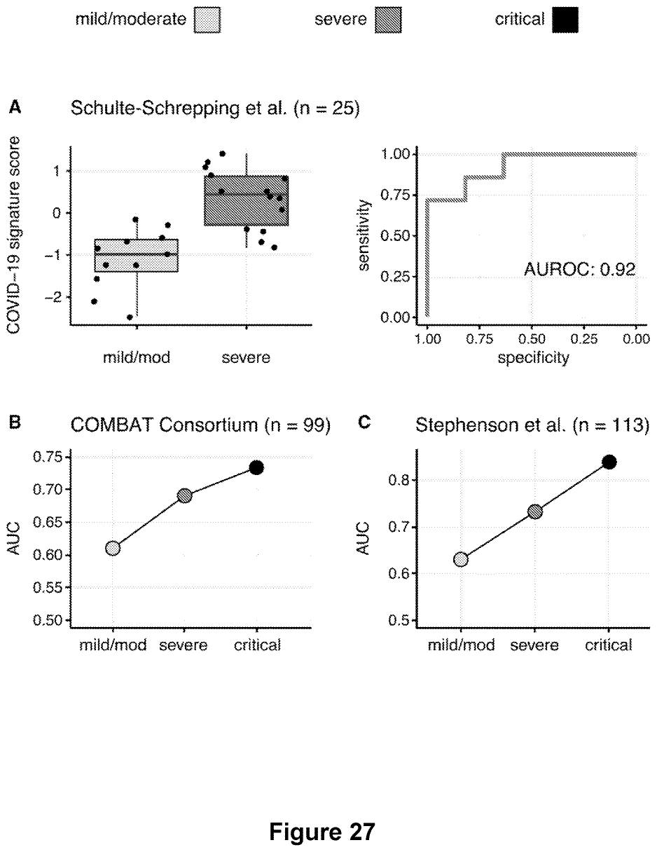

Having established a robust and specific COVID-19 signature, a determination of whether its performance varies with disease severity was investigated. COVID-19 patients show a wide diversity of disease severity, ranging from asymptomatic to critical. While information on severity in COVID-19 studies was generally sparse and highly heterogeneous, three large single cell datasets included detailed metadata on condition severity (COvid-19 Multi-omics Blood ATlas (COMBAT) Consortium, 2022; Schulte-Schrepping et al., 2020; and Stephenson et al., 2021). Within designations of severity that varied between these studies, the present disclosure defined three categories: mild/moderate, severe and critical disease. Depending on the study, mild/moderate also included asymptomatic cases and critical included subjects with an eventual death outcome. Analysis at the pseudo-bulk level showed that the COVID-19 signature score was higher for samples from more severe disease and discrimination between disease samples and healthy controls increases with disease severity (FIGS. 27A-27C). Altogether, the three studies confirmed a consistently positive association between the COVID-19 signature performance and COVID-19 severity.

### Immune Cell Type Signals Explain COVID-19 Signature Performance

In some embodiments, the systems and methods of the present disclosure identify a COVID-19 signature largely based on blood transcriptomes at the bulk level. Blood comprises diverse immune cell types whose proportions and transcriptional profiles can significantly change during infection. For example, COVID-19 patients show a decrease of peripheral blood subsets of both CD4+ and CD8+ T cells, and an increase of activated and differentiated effector cells (Bergamaschi et al., 2021). It was investigated whether signals from specific immune cells might explain the observed COVID-19 signature performance.

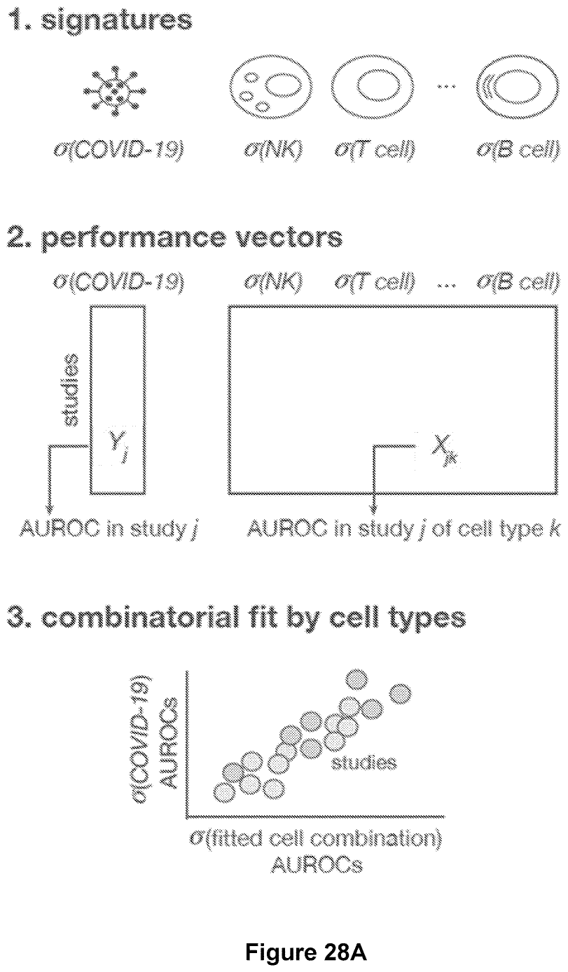

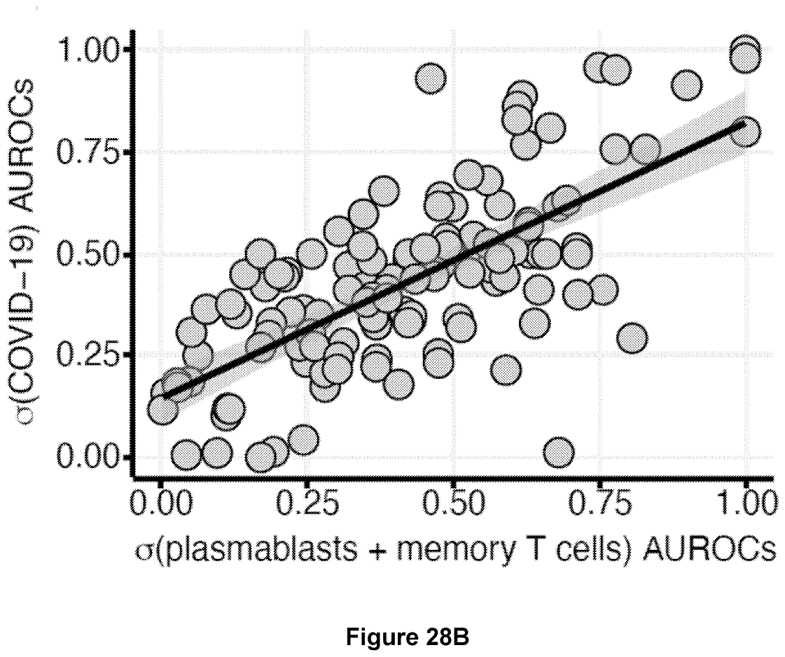

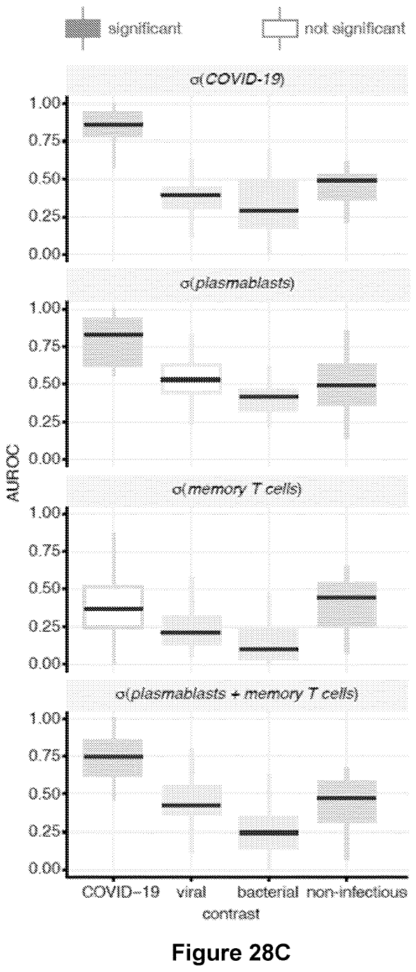

To address this question, a method based on three main steps was constructed (FIG. 28A). First, the present disclosure retrieved a set of immune cell type specific signatures from the Immune Response in Silico database (Abbas et al., 2005). Second, the COVID-19 signature and the database-derived cell type specific signatures were represented as performance vectors. The performance vector of a signature contains the AUROCs produced by that signature across all the studies, COVID-19 and cross-reactivity, in our curation. Third, a search for a minimal combination of cell type-specific signatures whose performance vector produced a maximum alignment with the performance vector of the COVID-19 signature was done (see Methods).

Using a greedy search algorithm, the present disclosure found that a combination of signatures associated with plasmablasts and memory T cells produced the best alignment with the COVID-19 signature (FIG. 28B). To assess whether the requirements on detection and cross-reactivity have been satisfied by the cell type signatures similar to the COVID-19 signature (FIG. 28C, first panel), the approach of hypothesis testing against an appropriate null hypothesis was followed, as above. The two cell types (plasmablasts, memory T cells) individually failed to simultaneously satisfy the requirements on detection and viral cross-reactivity. The plasmablast signature provided significant COVID-19 detection (AUROC=0.83, p-value=4.86·10-6), but the null hypothesis of viral cross-reactivity could not be rejected (AUROC=0.53, p-value=0.97, FIG. 28C, second panel). Conversely, memory T cells showed insignificant COVID-19 detection (AUROC=0.37, p-value=0.95), but the hypothesis of viral cross-reactivity with high significance could not be rejected (AUROC=0.21, p-value=1.60·10-11, FIG. 28C, third panel). Only when combined together, the two cell types satisfied the requirements of significant detection (AUROC=0.75, p-value=2.45·10-2) and no viral cross-reactivity (AUROC=0.43, p-value=3.66·10-2, FIG. 28C, fourth panel).

Overall, this analysis suggested that the COVID-19 signature performance may track a combined signal from plasmablasts and memory T cells.

### Single-Cell RNA Seq Data Support Plasmablasts as Mediators of COVID-19 Detection

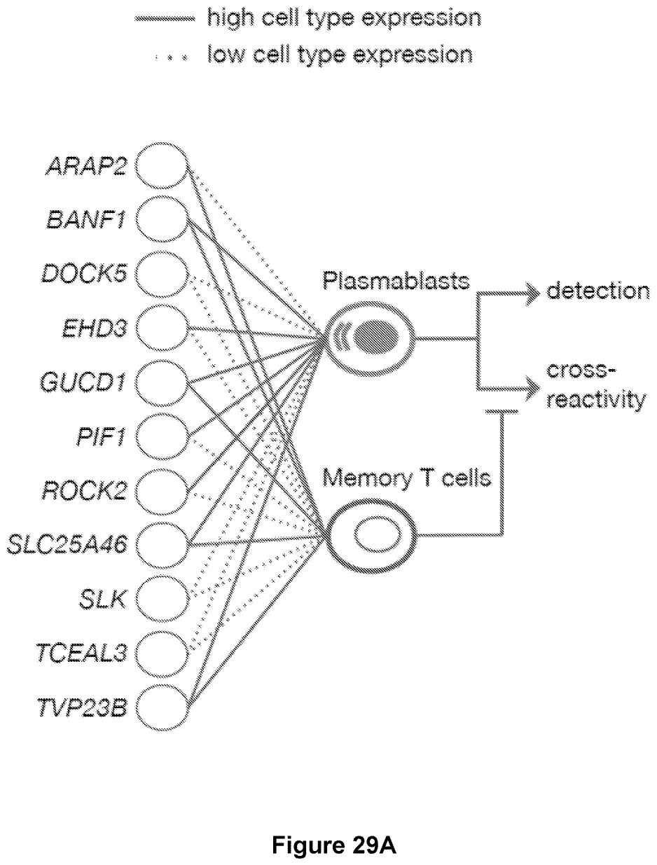

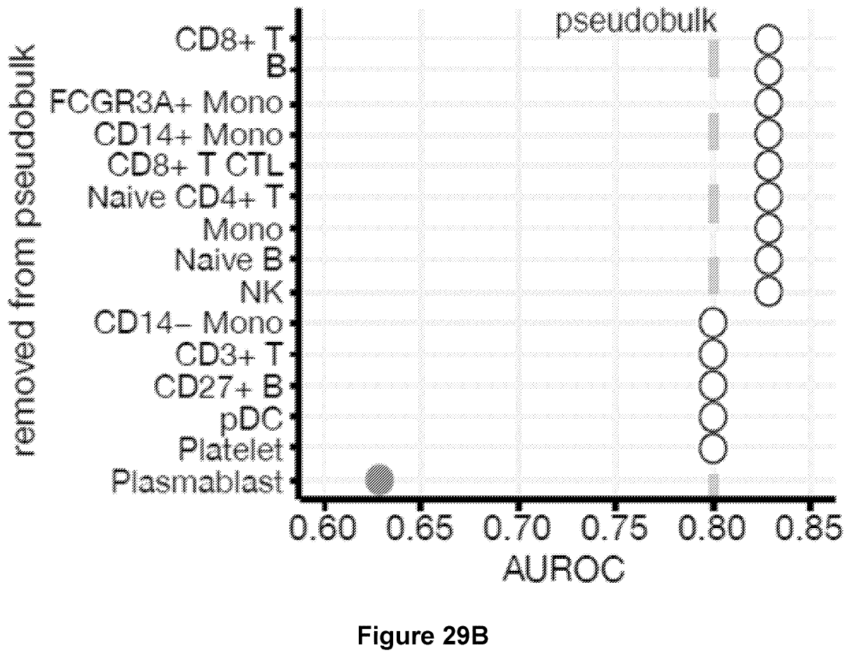

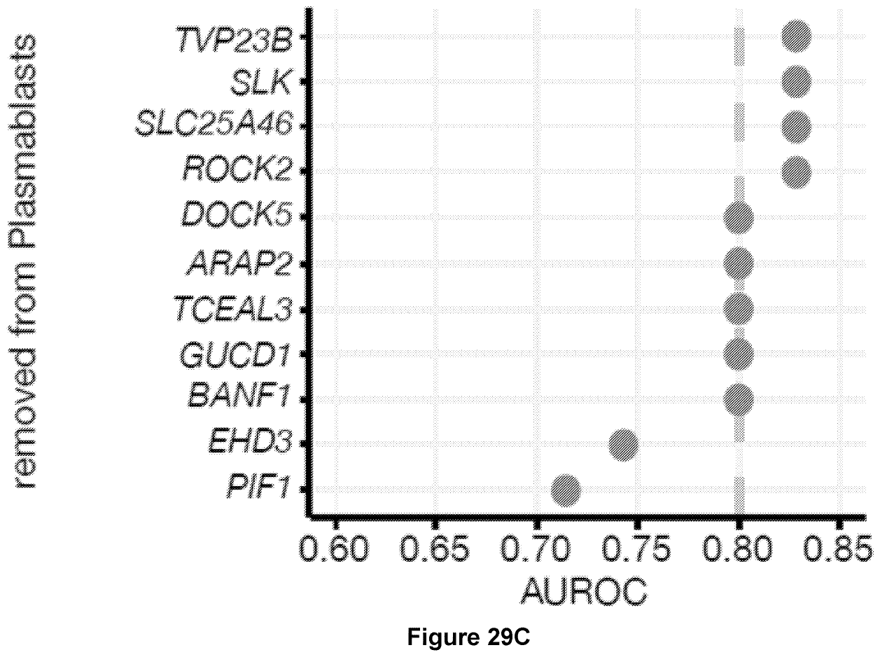

Next, the present disclosure aimed to build a global model of the COVID-19 signature performance by linking the signature genes with their specific expression in plasmablasts and memory T cells, supported by prior knowledge (Monaco et al., 2019) (FIG. 29A, Methods). The model was visualized as a bipartite weighted network whose nodes are the COVID-19 signature genes, plasmablasts and memory T cells, and whose edges correspond to cell type-specific expression levels. The resulting network showed that, out of the eleven COVID-19 signature genes, plasmablasts highly express seven genes and memory T cells highly express five genes.

To further investigate the role of plasmablasts and plasmablast-specific expression of signature genes in COVID-19 detection, the single-cell RNA-seq study containing PBMC gene expression profiles from seven COVID-19 infected subjects and five healthy controls were analyze (GSE155673, Arunachalam et al., 2020). The COVID-19 signature showed a good detection rate in this study when processed at the pseudo-bulk level (AUROC=0.80). The contribution of each cell type to COVID-19 detection was systematically explored by conducting a “leave-one-cell-type-out” analysis, where the present disclosure removed individual cell types during the construction of the pseudo-bulk matrix to evaluate their contribution to signature performance (see Methods). Removing the plasmablast compartment resulted in the largest reduction in performance of the COVID-19 signature compared to all other cell types (AUROC from 0.80 to 0.63, FIG. 29B), demonstrating a major role of plasmablasts as mediators of COVID-19 detection.

To explore the role of plasmablast-specific signature gene expression in detecting COVID-19, a “leave-one-gene-out” analysis restricted to the plasmablast compartment (see Methods) was conducted. Removing PIF1 and EHD3 expression from plasmablasts reduced COVID-19 signature performance at the pseudo-bulk level more than any other signature gene (AUROC from 0.80 to 0.72, FIG. 29C).

The results provide a minimal model of the signature performance, and identify plasmablasts' expression of PIF1 and EHD3 as the main contributor to COVID-19 detection.

### 4.4. Discussion

Using a novel computational framework, the present disclosure found a combination of 11 genes acting as a COVID-19 detection signature. Compared to previous studies (Aschenbrenner et al., 2021; Lee et al., 2020; McClain et al., 2021; Ng et al., 2021; Thair et al., 2021a), the signature development and subsequent validation were based on a larger and richer compendium of transcriptional studies, adding critical specificity and improving global applicability of the findings. Furthermore, the integration of epigenetic data, single-cell data, and prior knowledge improved the signature's overall robustness and interpretability.

The COVID-19 validation data included transcriptomes at bulk and pseudo-bulk levels, from both PBMC and whole blood. Despite these diverse data sources, the detection rate was consistently good (AUROC IQR of 0.7-0.9). A loss of performance was noted in one COVID-19 validation study (AUROC=0.57) (Thair et al., 2021a). Loss of performance in specific studies may originate from biases in demographic or clinical characteristics of the cohorts. One clinical characteristic analyzed in detail was disease severity. Based on three large studies with metadata on severity (COvid-19 Multi-omics Blood ATlas (COMBAT) Consortium, 2022; Schulte-Schrepping et al., 2020; and Stephenson et al., 2021), the COVID-19 signature appears to be very effective in detecting severe and critical cases, while being somewhat less sensitive to mild/moderate or asymptomatic cases. Without intending to be limited to any particular theory, it was hypothesized that this finding reflects a bias in the datasets used for signature derivation, which, early in the pandemic, tended to profile severe and critical COVID-19 patients. In addition to severity, various other metadata factors, such as time since infection, cases of co-infection and intubation status, may affect the generalizability of performance in unpredictable ways. In general, absence of comprehensive and well-structured metadata across studies limited our ability to identify conditions for optimal applicability.

Compared to previous work (Aschenbrenner et al., 2021; Lee et al., 2020; McClain et al., 2021; Ng et al., 2021; and Thair et al., 2021a), the cross-reactivity data included a wider diversity of viral and bacterial infections. Furthermore, unlike previous studies, the disclosed curation also contained data on comorbidities significantly associated with COVID-19, such as COPD, obesity, hypertension, and other risk factors (Bhaskaran et al., 2021; Williamson et al., 2020). These conditions may share inflammatory pathways also implicated in the host response to COVID-19. For example, severe COVID-19 presents with an increased release of pro-inflammatory cytokines, also observed in conditions associated with obesity that lead to systemic inflammation (de Lucena et al., 2020). To reduce signature cross-reactivity, host response signatures for COVID-19 need to be developed with an awareness of inflammatory processes that may pre-exist SARS-CoV-2 infection. The disclosed work here in part 4 of the present disclosure provides the first attempt to develop a COVID-19 signature while controlling for these frequent confounders. More broadly, the disclosed extensive data curation, combined with existing resources (Chawla et al., 2022) that include non-infectious inflammatory conditions can help improve the performance of future host response signatures for other infections.

Although signature performance was a primary objective, the disclosed framework leveraged additional data sources, such as pathway knowledgebase and ATAC-seq data, to increase its interpretability. Despite its limited size, the signature captured portions of antiviral pathways regulated in COVID-19 patients at the transcriptional level. Furthermore, the transcriptional regulation of the signature's genes was significantly correlated with their epigenetic regulation, showing convergent information from the two data sources. In particular, PIF1, which had the largest simultaneous transcriptional and epigenetic regulation, was also a major driver of COVID-19 detection in multiple independent cohorts. This indicated that evidence from multi-omics data can improve the selection of the signature genes.

To further increase the signature's mechanistic interpretability, our framework included a new approach to explain the performance of the signature in terms of signals from the different immune cells. The approach, conceptually related to previous work (Bolen et al., 2011), revealed that plasmablasts play a key role in COVID-19 detection. This notion, which was further corroborated by our analysis of scRNA-seq data, is also consistent with previous results showing a role for plasmablasts in antiviral responses (Fink, 2012; Turner et al., 2021). In COVID-19, large plasmablast expansions were noted as a characteristic feature early on in the pandemic and, more recently, have been found to be positively associated with COVID-19 disease severity (Schultheil3 et al., 2021). However, the findings of the present disclosure also clarified that a signature merely tracking plasmablast activity would produce a high degree of cross-reactivity with other viral infections. To control viral cross-reactivity, COVID-19 signatures should optimally include contributions from other immune cell types in addition to plasmablasts. Based on the disclosed analysis, a contribution from memory T cells aids in controlling cross-reactivity.

The ability to identify pathogen- and disease-specific signatures may pave new ways for differential diagnosis. The main advantage of host response diagnostic assays is the increased sensitivity early in the infection, when standard PCR diagnostic tests have poor sensitivity. The current study contributed to the development of a new host-response based COVID-19 diagnostic test (Cappuccio et al., 2022). In that work, the present disclosure found initial evidence that the host response assay is able to detect SARS-CoV-2 early after infection. This advantage of early detection has the potential to curb pathogen spread more efficiently than current diagnostic technologies.

Earlier research in host response based diagnostics developed signatures of multiple viral infections (Bongen et al., 2019; Sampson et al., 2017; Thair et al., 2021a, 2021b; Zheng et al., 2021), tuberculosis (Moreira et al., 2021; Roy Chowdhury et al., 2018; Sodersten et al., 2021; Warsinske et al., 2019), neonatal sepsis (Sweeney et al., 2018), graft survival (Azad et al., 2018), and asthma exacerbations (Lydon et al., 2019b). While the disclosed study shares conceptual similarities with these works, it extends the analytical framework in three main ways. First, it leverages multi-objective data and prior biological information, increasing robustness and interpretability. Second, it searches for optimal signatures using a global combinatorial search. While computationally more expensive, global optimization strategies such as genetic algorithms are more likely to enhance performance compared to greedy optimization strategies. Third, it explains the identified signature in terms of signals from specific immune cell types, giving a global, interpretable model of performance. With these improvements, the disclosed framework is readily transferable to develop robust and interpretable signatures for other pathogens and disease states.

### 4.5. Materials and Methods

### Classification and Usage of Transcriptomic Studies

The transcriptomics studies were classified in four main categories: COVID-19 contrasts, other viral contrasts (respiratory and non-respiratory), bacterial (respiratory and non-respiratory) contrasts, and non-infectious contrasts. A typical contrast included samples from diseased subjects and healthy controls, to enable the identification of differential responses induced by the disease. In the case of non-infectious contrasts, the present disclosure distinguished between health conditions that are COVID-19 comorbidities and demographic factors that can contribute to higher COVID-19 risk. In the latter case, a contrast involved two groups (e.g., male vs. female), one of which was taken as a base class for differential analysis.

Studies from the four categories were organized into training (discovery), development, and test (validation) sets. Studies were split to balance varied microarray platforms, microarray manufacturers, sample sizes, and other demographic data (e.g., cohort age) across the training, development and test sets. Non-infectious conditions were omitted from the development set to preserve as many studies as possible for signature validation. Three additional datasets were used for severity analysis. The full listing of studies and their use is given in Table 4.1, above.

### Pre-Processing of Transcriptional Data

Data for each study was downloaded from the public domain. When read count matrices were provided by the study authors, they were utilized directly. In the other cases, RNA-seq.fastq files were mapped using the STAR aligner (Dobin et al., 2013) and the count matrices were compiled using featureCounts (Liao et al., 2014). The counts were further processed in the following steps: Voom transformation (Law et al., 2014); quantile normalization; data shifting to enforce positivity of expression values. The latter step consisted of adding a positive constant to the expression matrix if it contained negative or zero values, so that the minimum expression value was one. Positivity of expression values was required for the calculation of the ‘gene signature score’, which involves geometric means of expression values (Andres-Terre et al., 2015) (see section “Calculating the AUROC given a signature and a transcriptional contrast”).

Single cell RNA-seq .fastq files were processed using the CellRanger pipeline. The pseudo-bulk RNA-seq dataset was created by summing all gene counts across cells after basic filtering for poor quality cells, doublets and low cell counts.

For downstream analysis of all gene expression data, transcripts not annotated as protein-coding were excluded. Finally, the pre-processed expression matrix and the meta-data of each study was reformatted to be compatible with the MetaIntegrator package (Haynes et al., 2017). All the bulk and single-cell datasets used in this study and their accession codes are listed in Table 4.1.

### Pre-Selection of Genes and Annotation Terms for the Optimization Framework

To preselect genes, a meta-analysis of the five COVID-19 transcriptional studies in the training set using the MetaIntegrator package (Haynes et al., 2017) was performed with following criteria: False Discovery Rate<0.05; minimum combined effect size>0.5; the option ‘numberStudiesThresh’ was set to five, to enforce a consistent significant regulation in all the COVID-19 training studies. The results of this meta-analysis is found in Table 4.2. Additional details and information regarding Table 4.2 is found at Cappuccio et al., “Multi-objective optimization identifies a specific and interpretable COVID-19 host response signature,” Cell Systems 13(12), pg. 989-1001; Supplementary Table 2, which is hereby incorporated by reference in its entirety for all purposes.

To preselect relevant annotation terms, the present disclosure performed GSEA (Subramanian et al., 2005) using the combined effect size from the meta analysis above as gene-level statistics. GSEA was done using the complete gene set, in combination with Reactome, (Jassal et al., 2020), ImmPort, (Bhattacharya et al., 2018), Iris (Abbas et al., 2009), DMAP (Novershtern et al., 2011) and CIBERSORT (Newman et al., 2015). Annotation terms with adjusted p-value<0.05 were considered significant and selected for downstream analysis. The pre-selected annotation terms can be found in Table 4.3. Additional details and information regarding Table 4.3 is found at Cappuccio et al., “Multi-objective optimization identifies a specific and interpretable COVID-19 host response signature,” Cell Systems 13(12), pg. 989-1001; Supplementary Table 3, which is hereby incorporated by reference in its entirety for all purposes.

### Analysis of ATAC-Seq Data

Bulk ATAC-seq data (preprint) from five healthy and four infected samples were aligned to hg38 using bowtie2 (Langmead and Salzberg, 2012). De-duplicated reads from all samples were pooled together and ATAC-seq peaks were called using MACS2 (Zhang et al., 2008) with peak q-value cutoff set at 0.05. Normalized read counts in peaks for each sample were extracted and calculated using HOMER (Heinz et al., 2010). A linear regression model was used to assess the correlation between chromatin accessibility changes and COVID-19/control phenotypes with sample sex as a covariate. For each peak, the regression p-value and a log 2 fold-change between healthy controls and COVID-19 samples were calculated. To facilitate the gene signature identification, the present disclosure did a gene orientated peak annotation. For each gene, the present disclosure looked for (1) peaks in the proximal promoter region (+/2 kb around TSS), (2) peaks in blood enhancers looping to gene promoters though 3D chromatin interactions (fenrir.flatironinstitute.org/) (Chen et al., 2021), and (3) the nearest peak if not overlapping with either the promoter or enhancers. Then, the present disclosure assigned the most differential peak's p-value and its fold change to that gene (Table 4.4). Additional details and information regarding Table 4.4 is found at Cappuccio et al., “Multi-objective optimization identifies a specific and interpretable COVID-19 host response signature,” Cell Systems 13(12), pg. 989-1001; Supplementary Table 4, which is hereby incorporated by reference in its entirety for all purposes.

For each gene j, the present disclosure combined the two quantities to form an overall ATAC-seq score as follows:

$${score}_{j}^{ATAC} = - {log}_{10} ( {pval}_{j} * f {c}_{j} )$$

where pvalⱼ and fcⱼ are respectively the p-value and fold-change of the peak assigned to gene j.

### Calculation of the AUROC Given a Signature and Transcriptional Contrast

A signature σ is a set of up-regulated genes σ(up) and a set of down-regulated genes σ(down). A transcriptional contrast is a pair (xₖ, yₖ), where xₖ is the vector of gene expression values in sample k, and yₖ is an associated binary label (e.g. COVID-19 versu healthy). Given a signature and a transcriptional contrast, the resulting area under the ROC curve (AUROC) is computed as described in Sweeney et al., 2016, and briefly summarized here. First, a signature score is computed for each sample in the study. The score is defined as the geometric mean of the expression values of σ(up) minus the geometric mean of the expression values of σ(down) genes. Second, the signature score is used to rank all the samples in the transcriptional contrast. The resulting ranking, paired with the binary labels y is then used to compute the study AUROC.

### Calculating the Multi-Objective Fitness

The multi-objective fitness recapitulates the different objectives of a signature: high detection in COVID-19 studies; low cross-reactivity with all non-COVID-19 contrasts; consistency with the additional data sources. In some embodiments, the systems and methods of the present disclosure now describes the formulation of each of these objectives.

### Formulating the Fitness for COVID-19 Detection

To quantify COVID-19 detection for a given signature a, the present disclosure forms the vector AUROC Covid-19(σ), whose components are the AUROCs produced by a with respect to the COVID-19 studies used for training. The fitness component related to COVID-19 detection—denoted by ƒ(σ)—is defined as the minimum of these AUROCs:

$${f}_{det} ( σ ) = min ( {AUROC}_{COVID - 19} ( σ )$$

The function ƒ_{det}(σ) takes on values in the range [0, 1], and is maximized at the value of 1.0 for any signature with perfect discrimination in all COVID-19 versus healthy used for training. Following the same functional definition, the present disclosure derives a component of the fitness function for direct contrasts between COVID-19 and other infections.

Formulating the Fitness for Cross-Reactivity with Non-COVID-19 Studies

When evaluating the signature in non-COVID-19 studies, the objective is to minimize cross-reactivity. Consider the cross-reactivity with respect to a class c of non-COVID-19 studies, such as contrasts involving respiratory infections. Denote by AUROC_{c}(σ), the vector of AUROCs produced by the signature σ in studies belonging to class c that are used for training. The goal is to define fitness rewarding signatures for which AUROC has all components less or equal than 0.5. An AUROC of 0.5 is consistent with random classification and corresponds to absence of cross-reactivity. Note that AUROC values below 0.5 are not problematic in terms of cross-reactivity. As an example, consider the limit case of a single gene X that is consistently up-regulated in COVID-19 contrasts, while consistently down-regulated in bacterial contrasts. A classifier based on this single gene would return AUROCs close to 1 in the COVID-19 contrasts, and AUROCs close to 0 in bacterial contrasts. This scenario is still perfectly consistent with the goal of minimizing cross-reactivity, because the gene regulation is qualitatively different in COVID-19 contrasts (up-regulated) compared to bacterial contrasts (down-regulated). For this reason, any AUROC value below 0.5 is considered as lacking cross-reactivity, and only values above 0.5 are penalized in the signature optimization.

As such, the fitness component related to cross-reactivity with respect to class c—denoted by ƒ(σ) is defined as follows:

$${f}_{c} ( σ ) = {min}_{contrasts in c} ( 1 - {2 [ {AURUC}_{c} ( σ ) - 0.5 ]}_{+} )$$

In the above formula, the symbol [x]+ denotes the positive part defined as:

$${[ x ]}_{+} = \{ x if x > 0 , 0 if x \leq 0 \}$$

The minimum is taken with respect to the available contrasts in class c used for raining. The fitness ƒ_{c}(σ) has values in the range [0, 1], where the value of 1.0 corresponds to an ideal signature producing no cross-reactivity in all the contrasts in class c. Following the same reasoning and functional definitions, the present disclosure derive components of the fitness function involving cross-reactivity with all the considered classes of contrasts.

Formulating the Consistency of a Signature with Respect to ATAC-Seq Data and Annotation Terms

To assess the consistency between a signature and the additional sources of information, the present disclosure uses the notion of projection. The signature σ can be represented as a binary vector whose components correspond to one of the pre-selected genes, and the value of the component is either one or zero depending on whether the gene belongs or does not belong to the signature. Similarly, the pre-selected annotation terms are represented as binary vectors, and the value of the component is either one or zero depending on whether the gene belongs or does not belong to the corresponding annotation term. Finally, the ATAC-seq gene-level scores are represented as vectors whose components are ordered in the same way as the components of the signature vector. The consistency of the signature σ with the additional sources provided by the annotation terms and the vector of ATAC-seq gene-level scores is computed as the mean of the scalar products between the signature vector and each of these vectors:

$$consistency ( \underline{σ} ) 〈 ( \frac{\underline{σ} \cdot {\underline{t}}_{1}}{‖ \underline{σ} ‖ \cdot ‖ {\underline{t}}_{1} ‖} , \frac{\underline{σ} \cdot {\underline{t}}_{2}}{‖ \underline{σ} ‖ \cdot ‖ {\underline{t}}_{2} ‖} , … , \frac{\underline{σ} \cdot {\underline{t}}_{K}}{‖ \underline{σ} ‖ \cdot ‖ {\underline{t}}_{K} ‖} , \frac{\underline{σ} \cdot {\underline{score}}^{ATAC}}{‖ \underline{σ} ‖ \cdot ‖ {\underline{score}}^{ATAC} ‖} ) 〉$$

In the above equation, the vector tₖ is the binary vector representation of the k^{th}annotation term; score ATAC is the vector of gene-by gene ATACseq scores (see above, section “analysis of ATAC-seq data”); and the symbol<⋅> denotes the average of the vector components in the parentheses.

By combining all the partial objectives described above, the multi-objective fitness corresponding to the signature σ consists of the following vector:+

$${f}_{k} ( σ ) = ( {f}_{k} ( σ ) , consistency ( σ ) )$$

where w₁, w₂, . . . , wₖ are non-negative weights. The procedure of linear scalarization corresponds to maximizing the family of scalar fitness functions F_{w}(σ; w₁, w₂, . . . , wₖ) for variable weights. In some embodiments, the systems and methods of the present disclosure considered weight combinations by letting the weights w vary in a suitable range of values. The choice of the grid points was driven by an initial exploratory analysis, and by the need to limit the computational cost. In some embodiments, the systems and methods of the present disclosure explored the following values: [0; 1] for the two groups of COVID-19 contrasts: COVID-19 vs healthy, and COVID-19 vs pathogens; [0.0; 0.33; 0.66; 1] for three classes of non-COVID-19 contrasts: respiratory viral and bacterial vs healthy; non-respiratory viral and bacterial versus healthy; non-infectious versus healthy; and [0; 1] for the consistency with other data sources. This produced a total of 2²·4³2=512 points. For each grid point, the present disclosure maximized the scalarized objective function using a genetic algorithm, as implemented in the R package GA. As optimization parameters, the present disclosure set a population of 200 solutions and 100 iterations. In some embodiments, the systems and methods of the present disclosure restricted the search to signatures satisfying additional constraints. First, the present disclosure focused on signatures containing less than twelve genes. Second, the present disclosure focused on signatures with an approximately balanced representation of up- and down-regulated genes. This constraint was imposed as follows:

$$\frac{max ( \vert {σ}_{up} \vert , \vert {σ}_{down} \vert )}{min ( \vert {σ}_{up} \vert , \vert {σ}_{down} \vert )} \leq 1.5$$

where |σᵤₚ| and |σ_{down}| denote the number of up- and down-regulated genes in the signature, and the maximum imbalance factor was set to 1.5. Third, the present disclosure imposed a constraint on the minimal overlap between the signatures' genes and the genes measured in the different studies in the compendium, typically generated with a wide variety of microarray platforms. In some embodiments, the systems and methods of the present disclosure filtered out signatures whose median overlap with the compendium of studies was less than nine genes. The population of feasible solutions produced by the genetic algorithm for each grid point were then pooled and globally analyzed to select the optimal signature.

### Stability Analysis of the Solution Space

In some embodiments, the systems and methods of the present disclosure conducted a global analysis, to assess the stability of the different genes in the overall solution space. For each gene i, the present disclosure defined a stability metric as the fraction sᵢ of solutions containing i:

$${s}_{i} = \frac{number of solutions containing gene i}{total number of solutions}$$

Based on the stability of the different genes, the present disclosure then defined the stability of the different signatures. The stability of a signature σ, denoted by S(σ), was defined as the mean stability of its member genes.

### Signature Selection

For each signature σ, the present disclosure computed the corresponding multi-objective performance vector separately for the sets of training and development studies, as described above (see section Calculating the multi-objective fitness). Let us denote by

$${f}_{k}^{train} ( σ ) and {f}_{k}^{dev} ( σ )$$

the multi-objective performance of σ in the training and development studies, respectively. Furthermore, let us denote by p* the ideal point corresponding to a vector with all components equal to one, which corresponds to the perfect detection in all COVID-19 studies and zero cross-reactivity in any other non-COVID-19 studies. To reduce the dimensionality of multi-objective performance vector, each signature was mapped to a 2D plane whose components were the Euclidean distances:

$${d}_{train} ( σ ) = dist ( {f}_{k}^{train} ( σ ) , p \text{*)} and {d}_{dev} ( σ ) = dist ( {f}_{k}^{dev} ( σ ) , p \text{*)}$$

The selected signature σ* showed consistently small dₜᵣₐᵢₙ(σ*) and d_{dev}(σ*). The selection of σ* was further substantiated by stability analysis (see section Stability analysis of the solution space). This showed that σ* contained a majority of highly stable genes, frequently selected also in other candidate signatures.

### Pathway and Network Analysis of the Identified Signature

To infer blood-specific functional associations among the COVID-19 signature genes, the signature was processed using the online resource HumanBase (hb.flatironinstitute.org/). The following parameters were used: maximum number of genes=0; and minimum interaction confidence=0.1. The tool was also used to retrieve gene-level annotation.

### Correlation Between the Transcriptional and Epigenetic Regulation of the Signature Genes by COVID-19

To quantify the correlation between the transcriptional and epigenetic regulation of the signature genes by COVID-19, the present disclosure first defined an mRNA score for each signature gene in analogy with the previously defined ATAC-seq scores (see section Analysis of ATAC-seq data). The mRNA score for the signature gene j was defined as

$${score}_{j}^{mRNA} = - {log}_{10} ( {FDR}_{j} ) * {ES}_{j}$$

where FDRⱼ and ESⱼ are respectively the pooled False Discovery Rate and the effect size (ES) of gene j resulting from the meta-analysis of COVID-19 training studies (see section Pre-selection of genes and annotation terms for the optimization framework). The correlation between the transcriptional and epigenetic regulation of the signature genes was then computed as the correlation between the vectors of scores

$${score}_{j}^{mRNA} and {score}_{j}^{ATAC} .$$

To assess the significance level of the correlation level obtained with the COVID-19 signature, the present disclosure performed a resampling analysis. In some embodiments, the systems and methods of the present disclosure generated 1000 signatures of eleven genes randomly extracted from the pool of 398 pre-selected genes (see section Pre-selection of genes and annotation terms for the optimization framework). The significance level was estimated as the fraction of randomly extracted signatures produced a correlation level larger than the one obtained with the COVID-19 signature.

### Signature Validation

To assess the generalization performance of the selected signature, additional COVID-19 and non-COVID-19 studies were retrieved from GEO and pre-processed following the same steps applied to the training and development studies (see section Pre-processing of transcriptional data). For each validation study, the corresponding AUROC was computed as previously described (see section Calculation of the AUROC given a signature and a transcriptional contrast).

Fitting the COVID-19 Signature with Cell Type-Specific Effects

The purpose of this analysis was to find a minimal combination of cell-type specific effects best correlating with the performance of the COVID-19 signature. As cell-specific signatures, the present disclosure used the IRIS database (PMID: 15789058) containing signatures for 22 immune cell types. Let us denote by {σ_{c}} the set of cell-specific signatures. Starting from this set, the present disclosure derived new signatures to represent the following effects: 1) depletion of a cell type, and 2) combination of two cell types, which are examined below.

### Signature Representing the Depletion of a Cell Type.

To represent the depletion of cell type c, the present disclosure derived a new signature, denoted by σ_{-c′}, which has the same genes in σ_{c} but considered as down-regulated instead of up-regulated.

### Signature Representing the Combination of Two Cell Types.

Given two cell types c₁, c, the present disclosure derived a new signature representing their combination, denoted by σ_{c}_{₁}_{+c}_{₂}. The signature σ_{c}_{₁}_{+c}_{₂} has up-regulated genes given by the set union of the genes up-regulated by the two cells.

Using the two rules above, one can obtain the signature corresponding to a generic model of cell type-specific effects. The generic model can be written as

$$m = {\sum }_{k} {a}_{k} {c}_{k}$$

where the coefficients αₖ take on three values: −1 (depletion of cell type k); 0 (no change of cell type k); 1 (increase of cell type k).

Next, in some embodiments, the systems and methods of the present disclosure obtain the model m that best approximated the performance of the COVID-19 signature σ*. Let us denote by AUROC(σ*) the vector of AUROCs given by σ* across all the studies in our curation. Similarly, let us denote by AUROC(m) the vector of AUROCs given by a generic model m of cell-specific effects. The model best explaining the performance of σ* was found as the one whose associated performance vector AUROC(m) produced the largest correlation with AUROC(σ*). The correlation was through a greedy search: at each iteration, the cell-type producing the largest increase in correlation was added to the model, till no further improvement was possible. In our application, the process stopped after two iterations, which corresponded to the sequential addition of plasmablasts and inactivated memory T cells.

### Part 4: Section B—Systems and Methods for Earlier Detection of SARS-CoV-2 Infection by Blood RNA Signature Microfluidics Assay

Early SARS-CoV-2 diagnosis is a key non-pharmacological strategy to contain the current pandemic. Nucleic acid amplification tests (NAATs), the reference standard for SARS-CoV-2 diagnosis, are poorly sensitive during the first four days after infection, with false negative rates estimated in the range 67%-100% 1. Here, the present disclosure implemented a new assay that shows increased sensitivity to SARS-CoV-2 infection during the early window of NAAT false-negativity.

Host response assays (HRAs) are emerging as a new paradigm for infection diagnosis 2, recently implemented to discriminate viral from bacterial infections 3,4, and to detect early respiratory viral illnesses 5. Unlike NAATs that target viral genetic material, HRAs target transcriptional alterations in the host blood. These alterations may become detectable by RT-PCR as early as 12 hours after viral challenge 6. Given the potentially higher sensitivity early in infection, the present disclosure set out to implement the first HRA for SARS-CoV-2 diagnosis.

In some embodiments, the systems and methods of the present disclosure leveraged the COVID-19 Health Action Response for Marines (CHARM), a prospective study that identified incident SARS-CoV-2 infection among US Marine recruits from May 12 through Nov. 5, 2020, 7,8. The cohort included 3249 predominantly young, male participants. Participants were typically tested by an FDA-approved NAAT for SARS-CoV-2 three times during an initial two-week quarantine, and then biweekly for six weeks during basic training. Most infected participants were asymptomatic at the first positive NAAT and none required hospitalization. During basic training, 45.1% of participants showed a SARS-CoV-2 NAAT positive result at one or more time points. The high infection rate, along with the longitudinal design, made the CHARM study highly instrumental for benchmarking a new SARS-CoV-2 diagnostic assay.

The strategy to develop a SARS-CoV-2 HRA followed four main steps: (1) bio-informatics-driven identification of a SARS-CoV-2 host response signature; (2) technical implementation; (3) cross-sectional benchmark, by comparing HRA and NAAT results from different participants at randomly selected time points; (4) longitudinal benchmark, by comparing HRA and NAAT repeated measures over time for the same participants.

The first challenge the present disclosure faced was to identify a host transcriptional response specific for SARS-CoV-2 infection. In some embodiments, the systems and methods of the present disclosure aimed to find a compact set of 40-50 genes whose expression in blood would indicate SARS-CoV-2-infection, but not related infections such as influenza. To address this problem, the present disclosure curated a compendium of public blood transcriptomes from 15 COVID-19 studies and from 112 studies on a wide variety of viral and bacterial infections. Furthermore, the compendium included transcriptomes on COVID-19 comorbidities (e.g., obesity, hypertension) and risk factors (e.g., age, sex) which might act as potential confounders. Applying a combination of meta-analysis and optimization techniques to the data compendium, the present disclosure identified 41 genes that together provided robust SARS-CoV-2 detection (ROC AUC 0.7-0.9), and low cross-reactivity with other infections and confounding factors (ROC AUC≤0.5).

Next, the present disclosure implemented a HRA with three main components: whole blood collection through a PAXgene® Blood RNA Tube (BD Biosciences, San Jose, CA, USA); measurement of the expression levels of the 41 transcripts on an integrated fluidic circuit; sample interpretation through a machine learning algorithm. The algorithm was based on a regularized logistic regression classifier taking as input the combined ex-pression levels of the 41 transcripts measured in a blood sample, and returning as output the sample interpretation in one of the following classes: SARS-CoV-2 positive; SARS-CoV-2 negative; inclusive, in case of highly uncertain interpretation. The algorithm was developed using a training set of 245 SARS-CoV-2 positive and 296 SARS-CoV-2 negative samples from the CHARM study. To control for viral cross-reactivity, the training set included 63 blood samples from subjects in a vaccine trial after H3N2 influenza virus challenge 9. During algorithm training, the influenza samples were treated as SARS-CoV-2 negative. In some embodiments, the systems and methods of the present disclosure performed extensive tests to ensure that the machine learning-generated interpretation calls were highly reproducible across sample technical replicates.

In some embodiments, the systems and methods of the present disclosure first assessed the HRA performance in a cross-sectional way. In some embodiments, the systems and methods of the present disclosure extracted samples from the SARS-CoV-2 positive (n=93) and negative (n=93) groups at random time points, disregarding the participants' testing history. All of these samples were from participants not contributing to the training data, to avoid leakage from the training to the benchmark data. Using a NAAT-based comparator as the reference standard, HRA had a PPA of 96.6% (95% CI, 90.7-98.9%), an NPA of 97.7% (95% CI, 92.2-99.4%). To assess cross-reactivity, the present disclosure used 33 additional influenza samples from subjects in the influenza vaccine trial cohort used for training. Two samples produced inconclusive HRA results, and the cross-reactivity rate was 4/31=12.9% (95% CI, 4.2-30.7%). Overall, the cross-sectional benchmark demonstrated a high concordance between HRA and NAAT results.

In some embodiments, the systems and methods of the present disclosure then performed a longitudinal benchmark by comparing HRA and NAAT repeated measures for the same participants over time. The goal of this assessment was to explore whether HRA could anticipate SARS-CoV-2 diagnosis compared to NAAT. Due to the absence of a reference standard for SARS-CoV-2 diagnosis prior to NAAT positivity, the present disclosure performed a validation study 10. In some embodiments, the systems and methods of the present disclosure reasoned that some study participants were infected before their first positive NAAT result, but undetected due to low NAAT sensitivity early in infection. First, the present disclosure defined groups of samples with higher and lower risk for NAAT early false negativity, based on phylogenetic and epidemiological evidence. Second, the present disclosure compared HRA results in the two groups. In the higher-risk group, HRA was positive before NAAT in 10 of 15 participants (66.6%). In the lower-risk group, HRA was positive in 0 of 8 participants (0%). The results support an earlier SARS-CoV-2 diagnosis using HRA as compared to NAAT (Fisher exact test, p=0.0027).

Limitations of our study include an unknown generalizability beyond young, healthy, male participants; some cross-reactivity with influenza and possibly with other infections such as other coronaviruses; lack of knowledge of when SARS-CoV-2 exposure occurred or of when NAAT would first turn positive with more frequent testing.

Since the beginning of the COVID-19 pandemic, several diagnostic technologies have been proposed, including surface-enhanced Raman spectroscopy and field-effect transistor based biosensors. Compared to these and other technologies, the main advantage of HRAs is the potentially higher sensitivity early in infection. This benefit should be assessed relative to the additional cost associated with blood draws. Although a cost-benefit analysis was be-yond the scope of our work, the present disclosure envisage scenarios where using an HRA may be cost-effective. These scenarios include, for example, hospitals and nursing homes where the need to ensure virus-free environments is of critical importance.

In some embodiments, the systems and methods of the present disclosure provides the first implementation of a SARS-CoV-2 HRA, and initial evidence that monitoring the host response can anticipate NAAT infection diagnosis.

### Part 5: Systems and Methods for Xnnet: An Interpretable Machine Learning System and Method Using Prior Knowledge

### Description:

Provided is a neural net method that incorporates pathway information so that the model developed is easily interpretable, unlike typical neural networks, and more likely to be generalizable, rather than emphasizing classification signals only in the training data. The disclosed model is compact, being based on a relatively small number of features. It solves following problems: 1) creates a neural network classifier that reveals how it is classifying 2) by using outside information, such as pathways, it applies to a more general classification problem than the data used for training it, 3) the classification basis for any subject can be directly determined, and 4) it creates a model using a limited number of features that balances high performance with high interpretability.

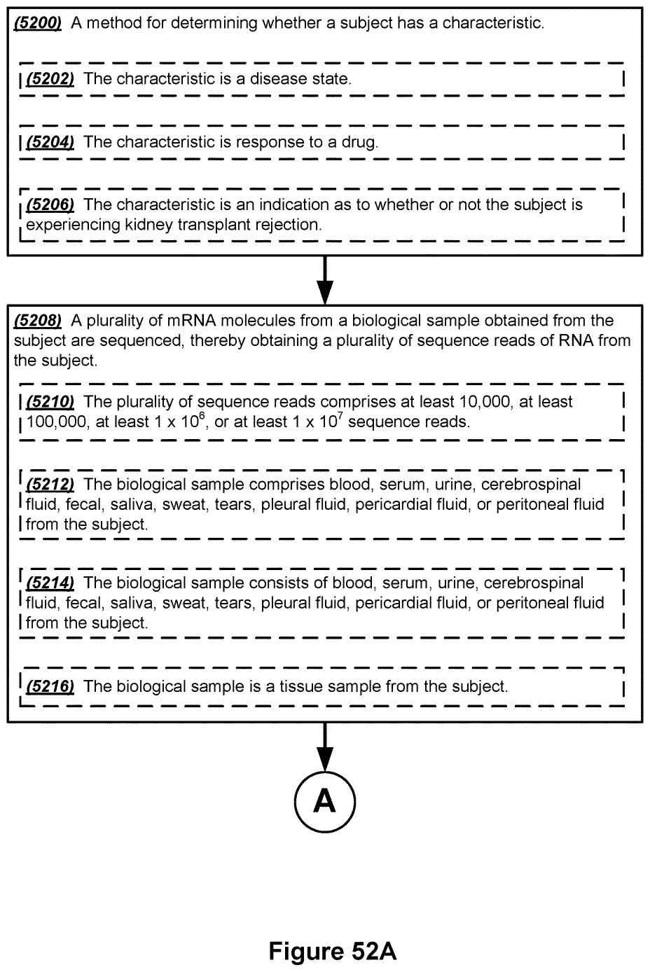

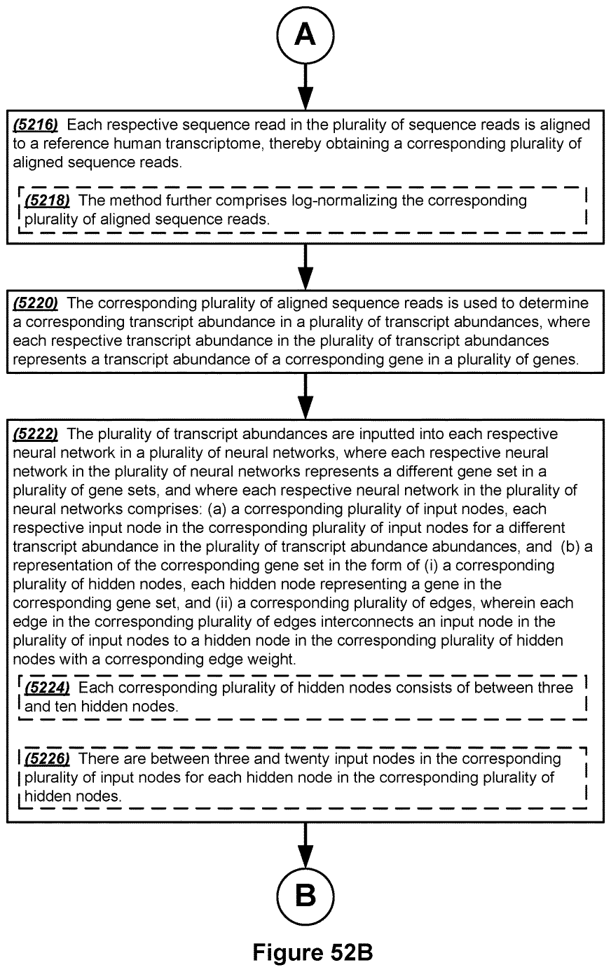

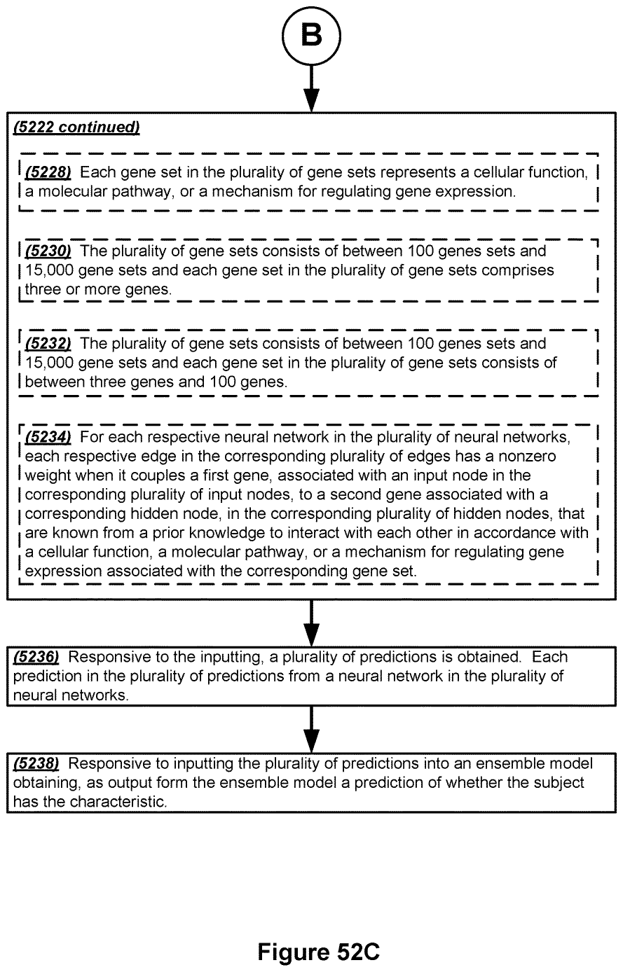

Accordingly, referring to block 5200 of FIG. 52A, one aspect in accordance with part 5 of the present disclosure provides a method for determining whether a subject has a characteristic. Referring to block 5202, in some embodiments, the characteristic is a disease state. Referring to block 5204, in some embodiments, the characteristic is response to a drug. Referring to block 5206, in some embodiments, the characteristic is an indication as to whether or not the subject is experiencing kidney transplant rejection.

Referring to block 5208, in the method, a plurality of mRNA molecules from a biological sample obtained from the subject are sequence, thereby obtaining a plurality of sequence reads of RNA from the subject. Referring to block 5210, in some embodiments, the plurality of sequence reads comprises at least 10,000, at least 100,000, at least 1×10⁶, or at least 1×10⁷ sequence reads. Referring to block 5212, in some embodiments, the biological sample comprises blood, serum, urine, cerebrospinal fluid, fecal, saliva, sweat, tears, pleural fluid, pericardial fluid, or peritoneal fluid from the subject. Referring to block 5214, in some embodiments, the biological sample consists of blood, serum, urine, cerebrospinal fluid, fecal, saliva, sweat, tears, pleural fluid, pericardial fluid, or peritoneal fluid from the subject.

Referring to block 5216 of FIG. 52B, in some embodiments, the biological sample is a tissue sample from the subject.

Referring to block 5216, in some embodiments, each respective sequence read in the plurality of sequence reads is aligned to a reference human transcriptome, thereby obtaining a corresponding plurality of aligned sequence reads.

Referring to block 5218, the method further comprises log-normalizing the corresponding plurality of aligned sequence reads.

Referring to block 5220, the corresponding plurality of aligned sequence reads is used to determine a corresponding transcript abundance in a plurality of transcript abundances, where each respective transcript abundance in the plurality of transcript abundances represents a transcript abundance of a corresponding gene in a plurality of genes.

Referring to block 5222, the plurality of transcript abundances are inputted into each respective neural network in a plurality of neural networks, where each respective neural network in the plurality of neural networks represents a different gene set in a plurality of gene sets, and where each respective neural network in the plurality of neural networks comprises: (a) a corresponding plurality of input nodes, each respective input node in the corresponding plurality of input nodes for a different transcript abundance in the plurality of transcript abundance abundances, and (b) a representation of the corresponding gene set in the form of (i) a corresponding plurality of hidden nodes, each hidden node representing a gene in the corresponding gene set, and (ii) a corresponding plurality of edges, wherein each edge in the corresponding plurality of edges interconnects an input node in the plurality of input nodes to a hidden node in the corresponding plurality of hidden nodes with a corresponding edge weight.

Referring to block 5224, in some embodiments, each corresponding plurality of hidden nodes consists of between three and ten hidden nodes.

Referring to block 5226, in some embodiments, there are between three and twenty input nodes in the corresponding plurality of input nodes for each hidden node in the corresponding plurality of hidden nodes.

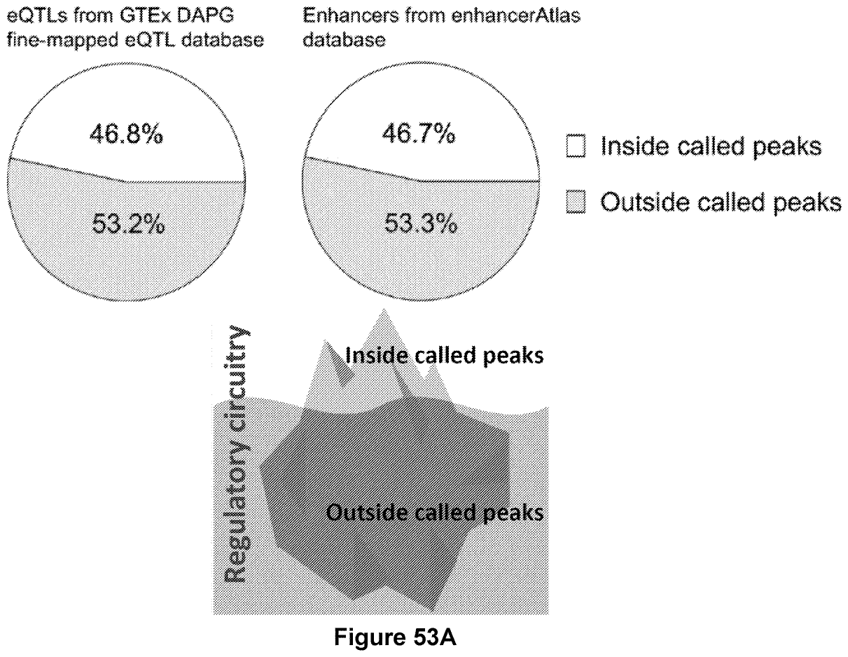

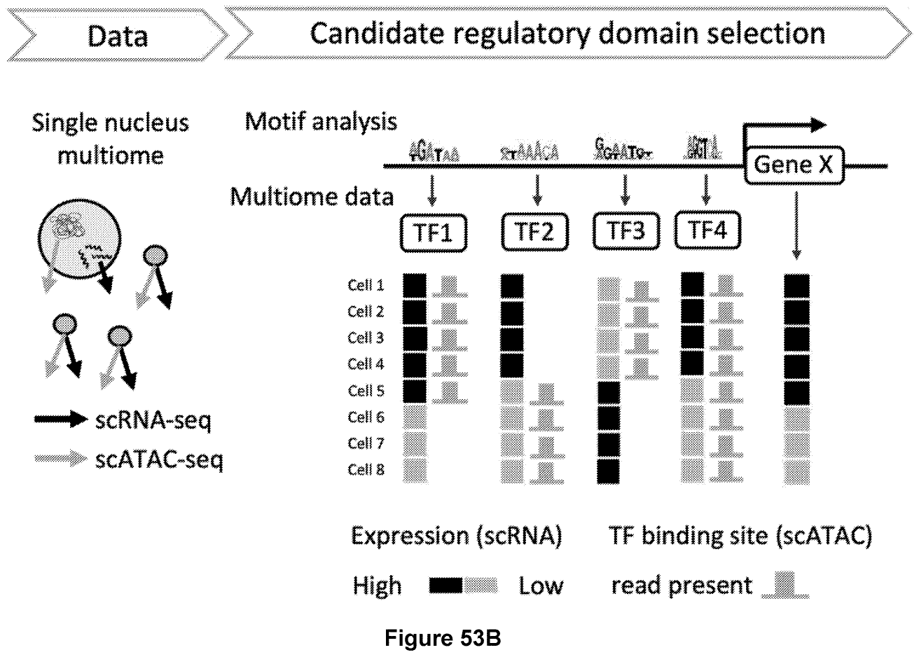

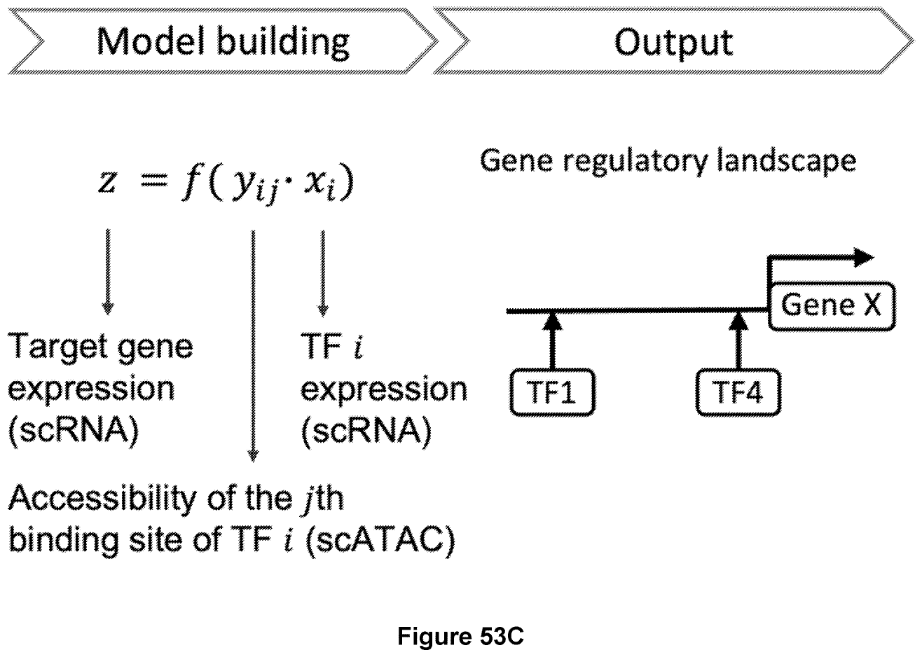

Referring to block 5228 of FIG. 53C, in some embodiments, each gene set in the plurality of gene sets represents a cellular function, a molecular pathway, or a mechanism for regulating gene expression.

Referring to block 5230, in some embodiments, the plurality of gene sets consists of between 100 genes sets and 15,000 gene sets and each gene set in the plurality of gene sets comprises three or more genes.

Referring to block 5232, in some embodiments, the plurality of gene sets consists of between 100 genes sets and 15,000 gene sets and each gene set in the plurality of gene sets consists of between three genes and 100 genes.

Referring to block 5234, in some embodiments, for each respective neural network in the plurality of neural networks, each respective edge in the corresponding plurality of edges has a nonzero weight when it couples a first gene, associated with an input node in the corresponding plurality of input nodes, to a second gene associated with a corresponding hidden node, in the corresponding plurality of hidden nodes, that are known from a prior knowledge to interact with each other in accordance with a cellular function, a molecular pathway, or a mechanism for regulating gene expression associated with the corresponding gene set.

Referring to block 5236, responsive to the inputting, a plurality of predictions is obtained. Each prediction in the plurality of predictions from a neural network in the plurality of neural networks.

Referring to block 5238, responsive to inputting the plurality of predictions into an ensemble model obtaining, as output form the ensemble model a prediction of whether the subject has the characteristic.

Another aspect in accordance with part 5 of the present disclosure provides is a computer system for determining whether a subject has a characteristic. The computer system comprises: one or more processors and memory addressable by the one or more processors. The memory stores at least one program for execution by the one or more processors. The at least one program comprises instructions for aligning each respective sequence read in a plurality of sequence reads, wherein the plurality of sequence reads represent a plurality of mRNA molecules in a biological sample obtained from the subject, to a reference human transcriptome, thereby obtaining a corresponding plurality of aligned sequence reads.

The at least one program further comprises instructions for using the corresponding plurality of aligned sequence reads to determine a corresponding transcript abundance in a plurality of transcript abundances, wherein each respective transcript abundance in the plurality of transcript abundances represents a transcript abundance of a corresponding gene in a plurality of genes.

The at least one program further comprises instructions for inputting the plurality of transcript abundances into each respective neural network in a plurality of neural networks, wherein each respective neural network in the plurality of neural networks represents a different gene set in a plurality of gene sets, and wherein each respective neural network in the plurality of neural networks comprises: (a) a corresponding plurality of input nodes, each respective input node in the corresponding plurality of input nodes for a different transcript abundance in the plurality of transcript abundance abundances, and (b) a representation of the corresponding gene set in the form of (i) a corresponding plurality of hidden nodes, each hidden node representing a gene in the corresponding gene set, and (ii) a corresponding plurality of edges, wherein each edge in the corresponding plurality of edges interconnects an input node in the plurality of input nodes to a hidden node in the corresponding plurality of hidden nodes with a corresponding edge weight,

The at least one program further comprises instructions for, responsive to the inputting, obtaining a plurality of predictions, each prediction in the plurality of predictions from a neural network in the plurality of neural networks.

The at least one program further comprises instructions for, responsive to inputting the plurality of predictions into an ensemble model obtaining, as output form the ensemble model, a prediction of whether the subject has the characteristic.

Another aspect in accordance with part 5 of the present disclosure provides a non-transitory computer readable storage medium. The non-transitory computer readable storage medium stores instructions, which when executed by a computer system, cause the computer system to perform a method for determining whether a subject has a characteristic. The method comprises aligning each respective sequence read in a plurality of sequence reads, wherein the plurality of sequence reads represent a plurality of mRNA molecules in a biological sample obtained from the subject, to a reference human transcriptome, thereby obtaining a corresponding plurality of aligned sequence reads;

The method further comprises using the corresponding plurality of aligned sequence reads to determine a corresponding transcript abundance in a plurality of transcript abundances, wherein each respective transcript abundance in the plurality of transcript abundances represents a transcript abundance of a corresponding gene in a plurality of genes.

The method further comprises inputting the plurality of transcript abundances into each respective neural network in a plurality of neural networks, wherein each respective neural network in the plurality of neural networks represents a different gene set in a plurality of gene sets, and wherein each respective neural network in the plurality of neural networks comprises: (a) a corresponding plurality of input nodes, each respective input node in the corresponding plurality of input nodes for a different transcript abundance in the plurality of transcript abundance abundances, and (b) a representation of the corresponding gene set in the form of (i) a corresponding plurality of hidden nodes, each hidden node representing a gene in the corresponding gene set, and (ii) a corresponding plurality of edges, wherein each edge in the corresponding plurality of edges interconnects an input node in the plurality of input nodes to a hidden node in the corresponding plurality of hidden nodes with a corresponding edge weight.

The method further comprises, responsive to the inputting, obtaining a plurality of predictions, each prediction in the plurality of predictions from a neural network in the plurality of neural networks.

The method further comprises, responsive to inputting the plurality of predictions into an ensemble model, obtaining, as output form the ensemble model, a prediction of whether the subject has the characteristic.

### 5.1 Introduction

Machine learning (ML) may revolutionize healthcare by assisting and ultimately automating medical decisions in diagnostics, health monitoring, and precision treatments. The quality of ML models is generally evaluated by performance metrics such as prediction accuracy on validation data. Previous work showed that ML classifiers, notably artificial neural networks, can solve medical classification problems with high accuracy, including near human-level performance.

While reaching high performance is a necessary property of ML models, it may not be sufficient to guide medical decisions. Accurate ML models can still lack human-level interpretability. If the criteria underlying predictions are obscure, their use in real-world problems is severely limited. The ability to explain ML predictions will increase trust in future ML models and their overall applicability.

An emerging challenge is generating ML models able to provide high performance while still preserving human-level interpretability. A key element of interpretable models is the integration of prior information and domain knowledge. The analysis of -omics data makes extensive use of domain knowledge in the form of gene annotation libraries and protein interaction networks (Avey et al. 2017; Mao et al. 2019).

Building on this idea, the present disclosure provides xnnet, a new framework for interpretable ML that combines prior knowledge, powerful bioinformatics analysis tools, and ensemble modelling. Xnnet achieves state-of-the-art performance on benchmark datasets, and results in highly interpretable decisions.

### 5.2 Results

### 5.2.1 Building Xnnet: a Super-Learner of Interpretable Neural Networks

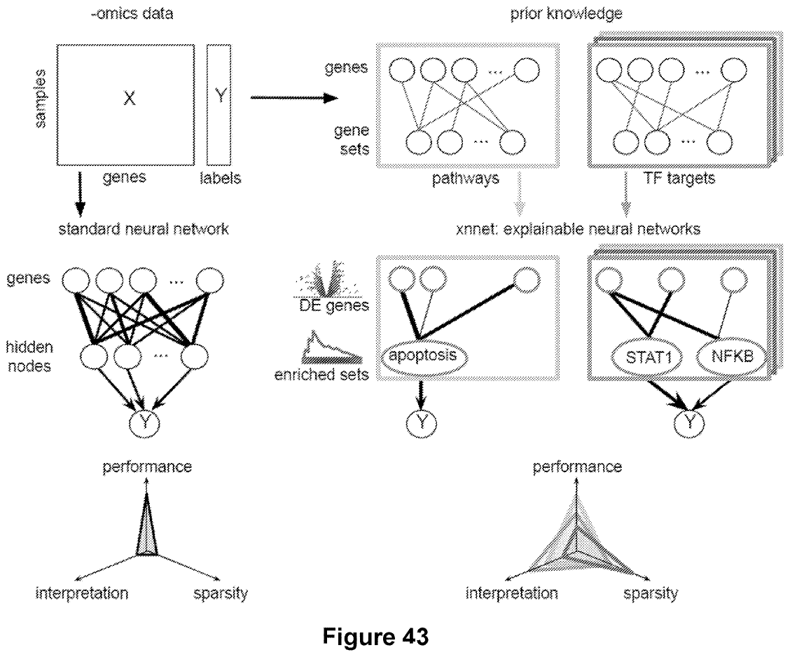

Consider the problem of predicting a patient's characteristic, such as a disease state or the response to a drug, based on a transcriptional profile of a tissue. Neural networks are highly instrumental for this purpose. They use the different genes as input nodes, and a set of hidden nodes to capture non-linearities between the inputs and the outcome of interest. While typically achieving high predictive power, standard neural networks can be complex, retaining many nodes and all possible edges in the solution. Furthermore, neither the hidden nodes nor the edges carry specific biological information, which obscures the criteria behind the classification (FIG. 43, left panel).

To overcome these limitations, the systems and methods of the present disclosure in accordance with part 5 integrates domain knowledge in the form of gene annotation libraries. These contain gene sets that cover a broad range of cellular functions, pathways, and mechanisms that regulate gene expression. By default, a compendium of the most established annotation libraries including over 12,000 gene sets (Table 5.1) is used, and user-defined gene sets can also be leveraged.

TABLE 5.1

| Library | Terms |
|---|---|
| BioCarta_2016 | 237 |
| BioPlanet_2019 | 1510 |
| ChEA_2016 | 645 |
| Chromosome_Location | 386 |
| ENCODE_Histone_Modifications_215 | 412 |
| ENCODE_TF_ChIP-seq_2015 | 816 |
| Genome_Browser_PWMs | 615 |
| GO_Biological_Process_2018 | 5103 |
| GO_Molecular_Function_2018 | 1151 |
| KEGG_2016 | 293 |
| NCI-Nature_2016 | 209 |
| Reactome_2016 | 1530 |
| TargetScan microRNA_2017 | 683 |
| TRANSFAC_and_JASPAR_PWMs | 326 |
| TRRUST_Transcription_Factors_2019 | 571 |
| WikiPathways_2016 | 437 |

Each of the libraries listed in the left hand column of Table 5.1 is downloadable from the Internet at maayanlab.cloud/Enrichr/index.jsp #libraries.

For each annotation library, the systems and methods in accordance with the present disclosure builds one or more base learners consisting of sparse, easily interpretable neural networks (FIG. 43, right panel). The input nodes are genes, the hidden nodes are gene sets, and edges between genes and gene sets are present only if supported by prior information, which vastly reduces the network complexity.

Transcriptomics datasets include tens of thousands of input genes, and annotation libraries typically contain hundreds of gene sets. A key difficulty is to distill the most relevant genes and gene sets for the network definition distinguishing the two classes of interest (e.g., healthy versus disease). To achieve this, systems and methods in accordance with the present disclosure process the data with powerful bioinformatics tools including differential expression, Gene Set Enrichment Analysis (GSEA), and a weighted set cover algorithm (see Methods).

Finally, systems and methods in accordance with the present disclosure make predictions that are based on a “super learner”, an ensemble model that aggregates predictions from neural networks derived from all the annotation libraries. As the present disclosure demonstrates, the performance of the ensemble model is superior to that of the individual networks, and improves on state of the art interpretable ML models.

Overall, the systems and methods of the present disclosure return a collection of interpretable, sparse classifiers that leverage the established prior biological knowledge and state-of-art bioinformatics analyses of transcriptomics data to discover a small set of pathways and regulatory processes driving the classification.

5.2.2 Xnnet Performance is Consistent with State-of-the-Art Interpretable Classification

The performance of systems and methods in accordance with the present disclosure was evaluated on three benchmark classification problems previously analyzed with LogMiNeR, an interpretable machine learning algorithm based on network-constrained logistic regression (Avey et al. 2017). The classification problems use blood transcriptomics data to discriminate the following groups: 1) subjects with systemic lupus erythematosus (SLE) versus control subjects (Bienkowska et al. 2014); 2) subjects with active tuberculosis vs. subjects with latent tuberculosis (Kaforou et al. 2013); 3) subjects with idiopathic dilated cardiomyopathy vs. subjects with ischemic heart disease (Liu et al. 2015). The three problems cover diverse biomedical applications and are of variable difficulty levels.

For each benchmark classification problem, the present disclosure measured the performance of 23 base neural networks derived from 18 annotation libraries (Table 5.1, FIG. 45) along with the ensemble model. In some embodiments, the systems and methods of the present disclosure fixed the size of each base network to include five hidden nodes and five input genes per hidden node. Furthermore, each network included an extra hidden node of ‘unassigned genes’. This includes top differentially expressed genes between the classes which are not the input of other hidden nodes (see Methods). Consistent with Avey et al. 2017, the present disclosure quantified performance in a robust manner, by generating a distribution of cross-validation accuracies for 50 random splits of the data in a training and test set (see Methods).

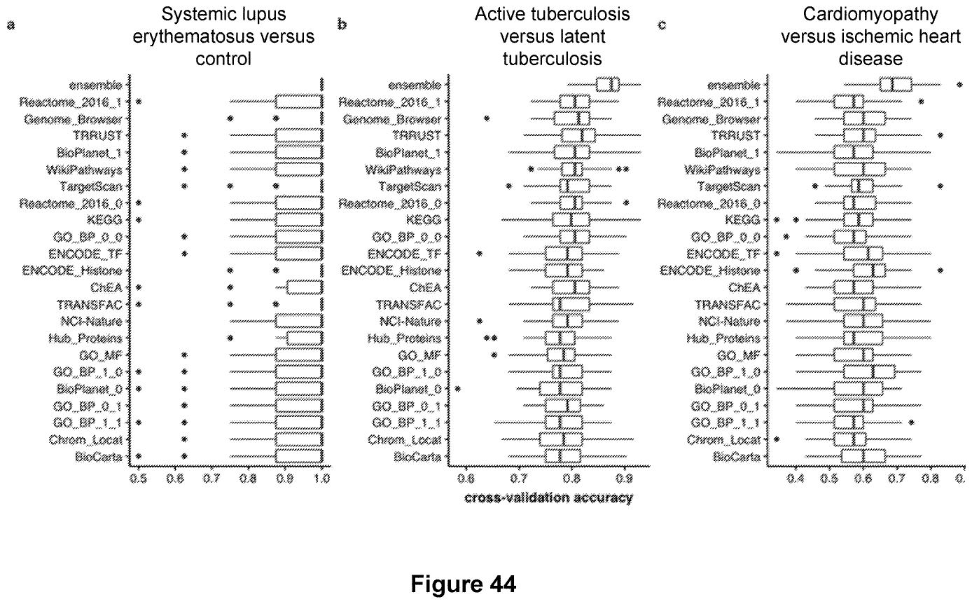

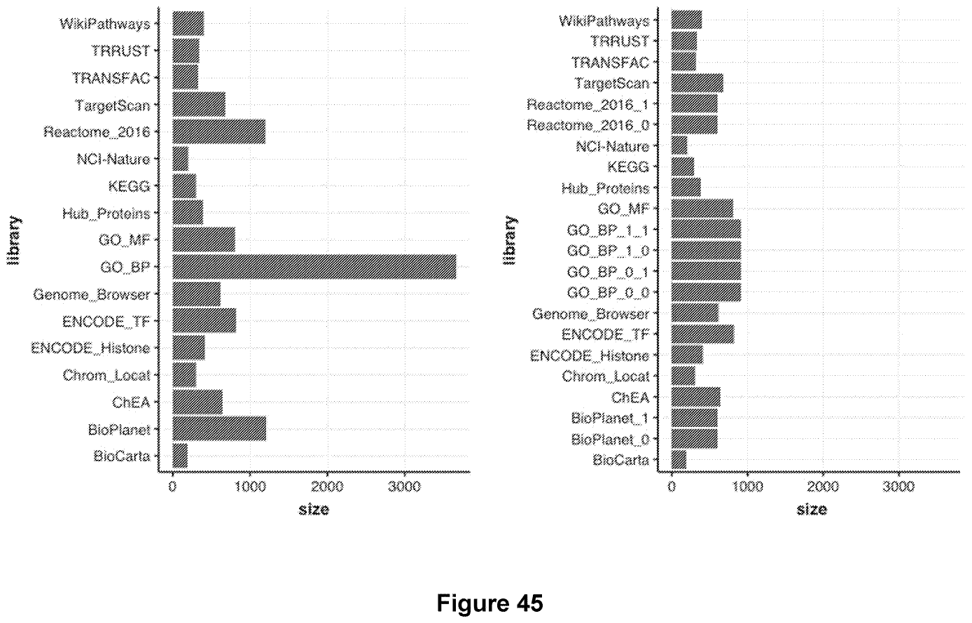

The ensemble classifier systematically improved the accuracy distribution observed with the individual neural networks (FIG. 44), suggesting that the different annotation libraries capture complementary aspects of the data, and synergize upon aggregation. Xnnet median accuracy was higher than LogMiNeR for all datasets. In particular, xnnet achieved a perfect discrimination of SLE subjects from control subjected in all the 50 random splits of the original data, which was higher than LogMiNeR (top median accuracy=98.3%, top IQR=98.0%-98.4%). In the discrimination of active vs. latent tubercolosis, xnnet produced a median accuracy of 87.5% (IQR=84.7-90.3%), higher than LogMiNeR (top median accuracy=85.4%, top IQR=85.0%-85.9%). Finally, xnnet the discrimination of idiopathic dilated cardiomyopathy vs. ischemic heart disease had median accuracy of 68.6%, (IQR=65.7%-74.3%), outperforming LogMiNeR (top median accuracy=66.5%, top IQR=64.4%-68.3%).

Altogether, these results demonstrate that xnnet outperforms state-of-the-art interpretable classification on a range of benchmark datasets.

### 5.2.3 Interpretable Prediction of Kidney Transplant Rejection

Next, the present disclosure aimed to test xnnet's ability to elucidate the classification process. As a case study, the present disclosure used a dataset previously generated to diagnose patients rejecting kidney transplant based on transcriptional profiles from renal biopsies (Reeve et al. 2013; Reeve et al. 2017). The goal was to derive an interpretable classifier providing a core set of biological and regulatory processes distinguishing patients resulting in kidney transplant rejection vs. no rejection. To this end, the present disclosure combined all samples associated with kidney transplant rejection, regardless of the particular rejection mechanism (see Methods).

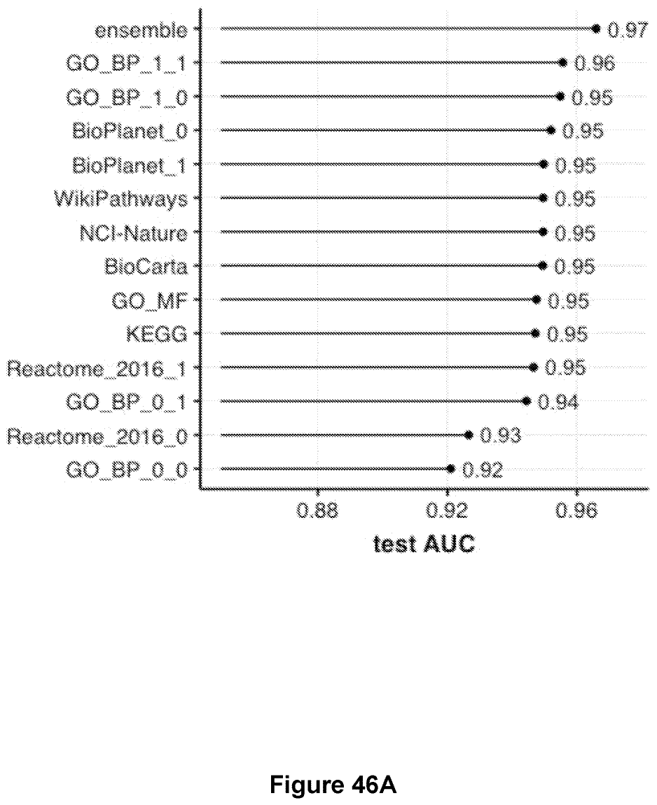

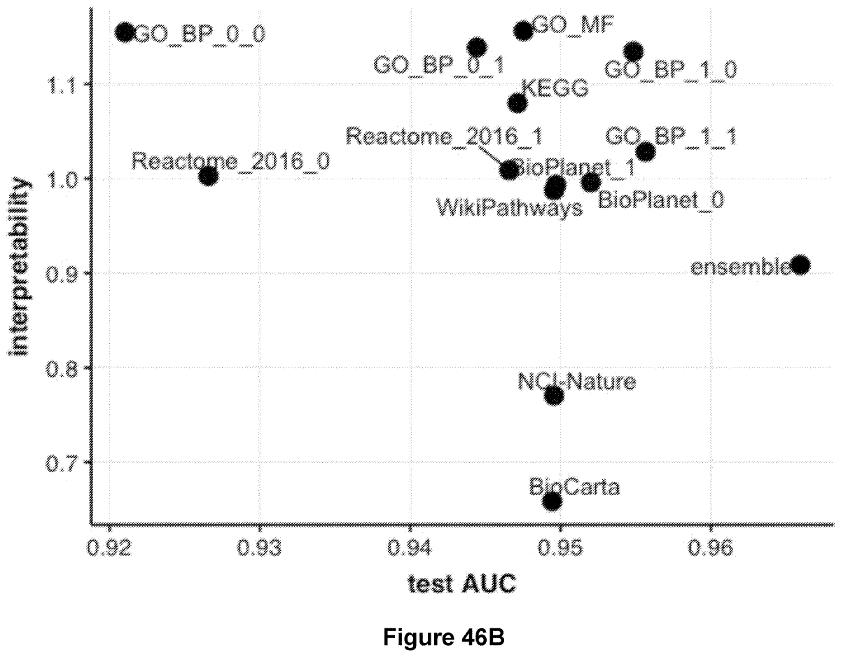

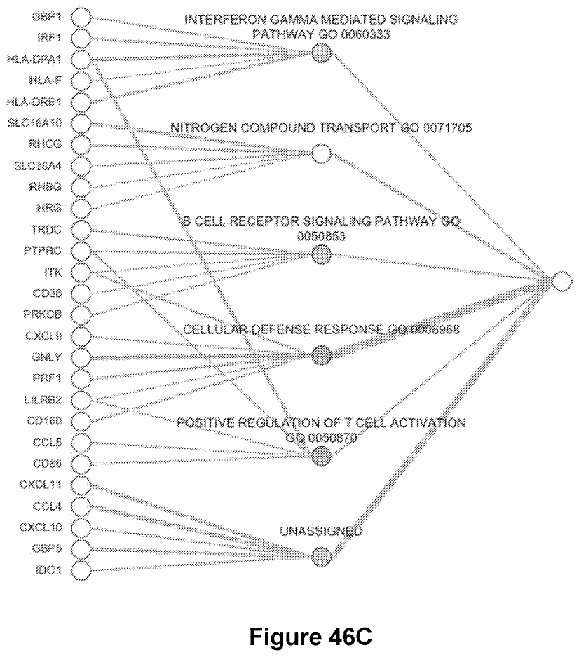

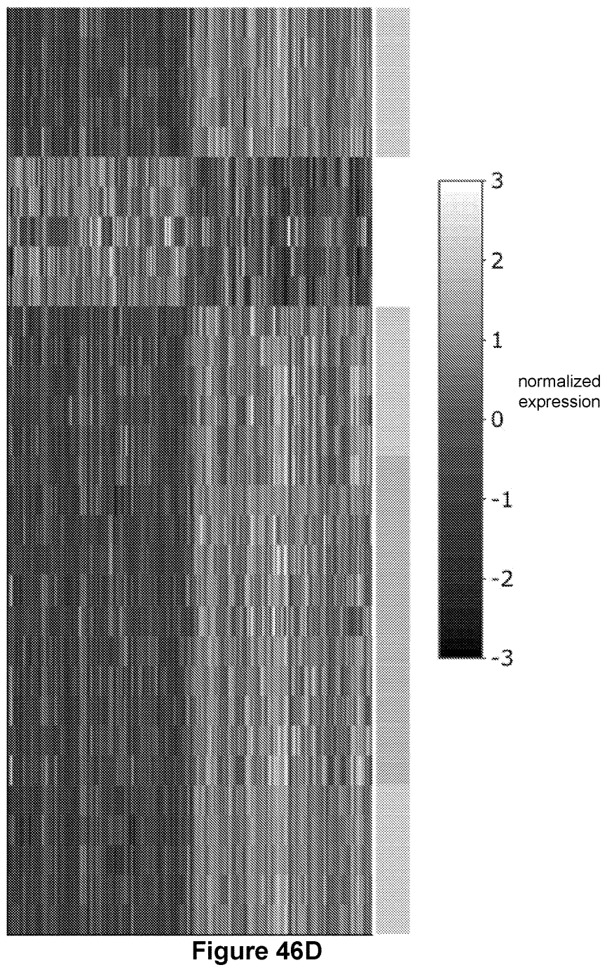

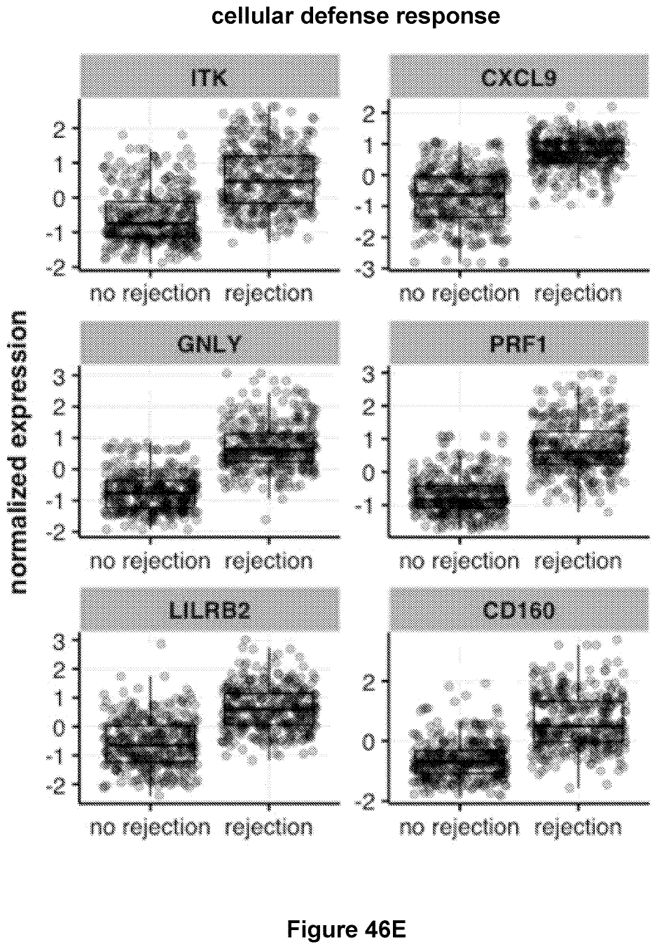

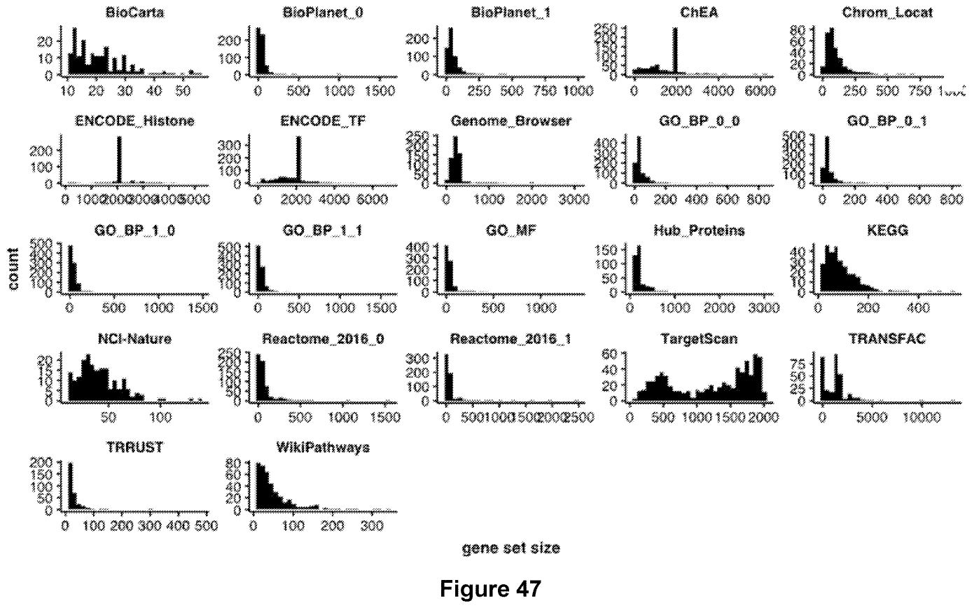

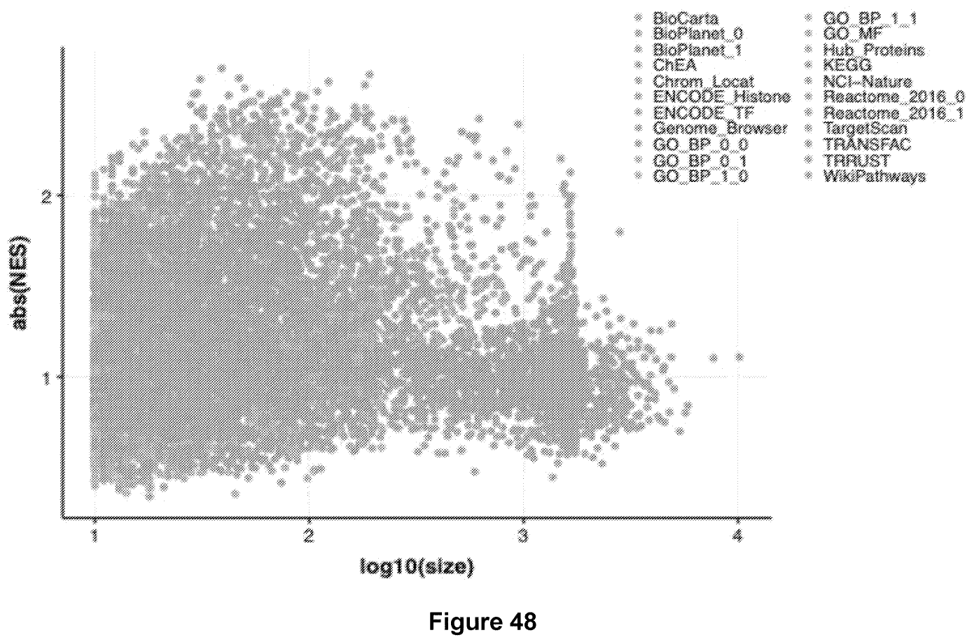

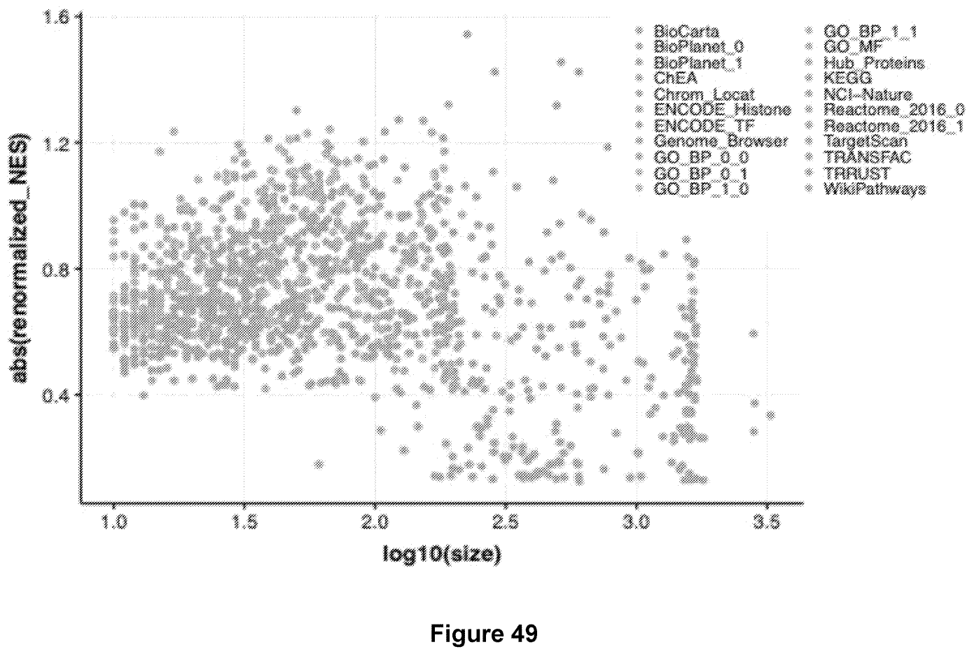

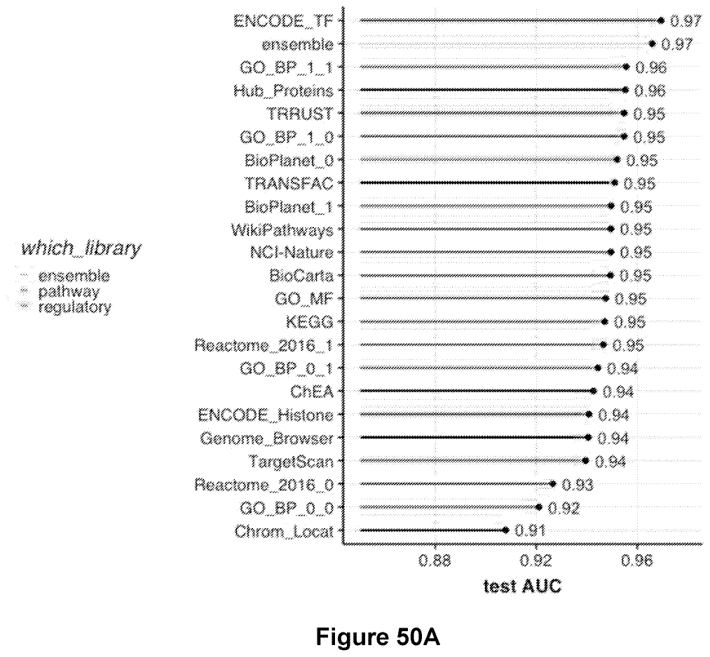

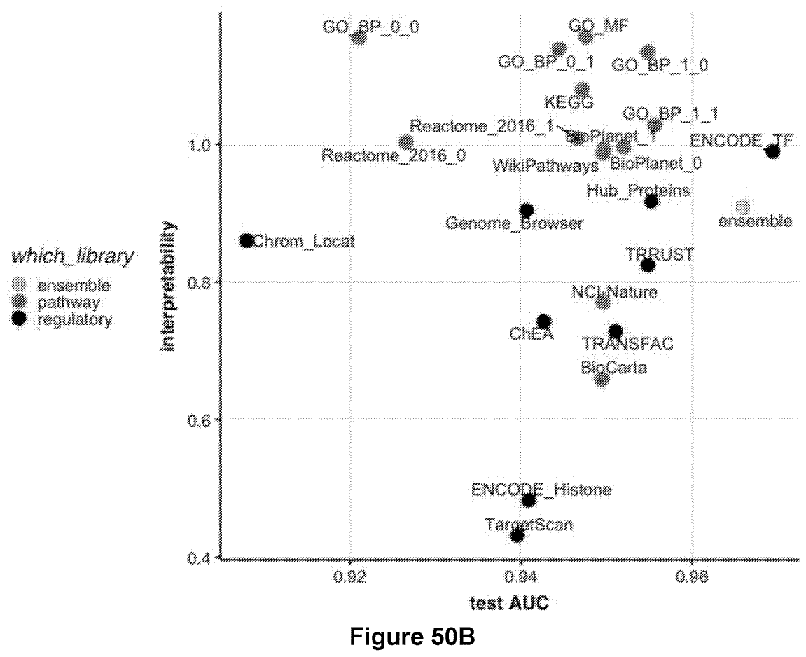

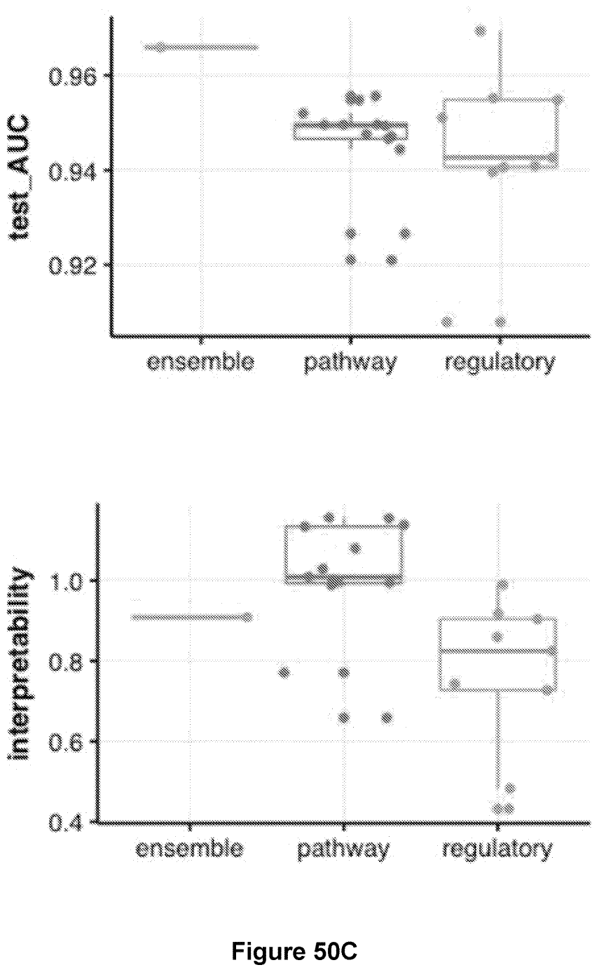

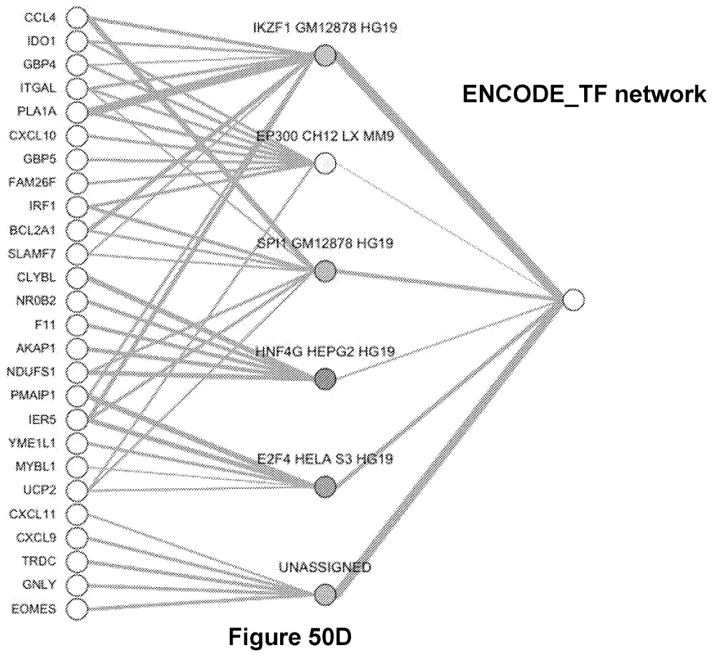

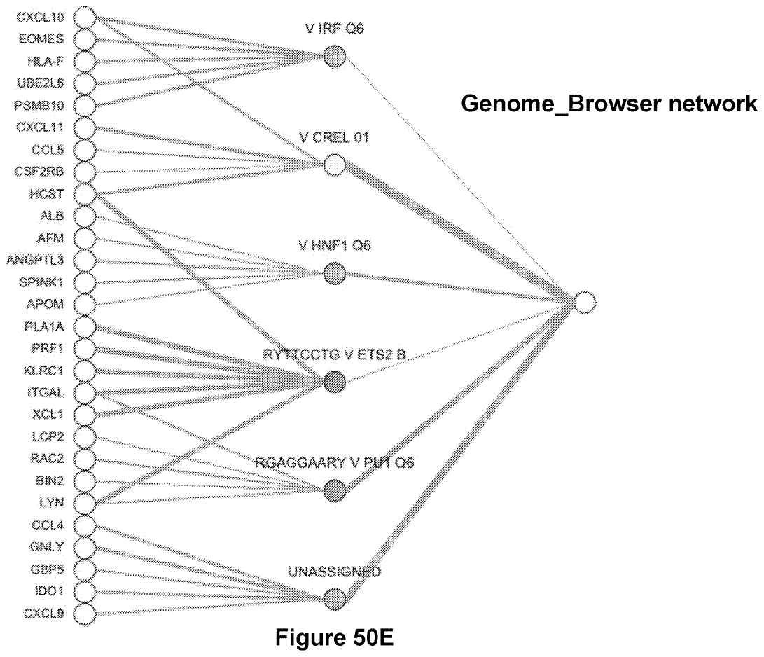

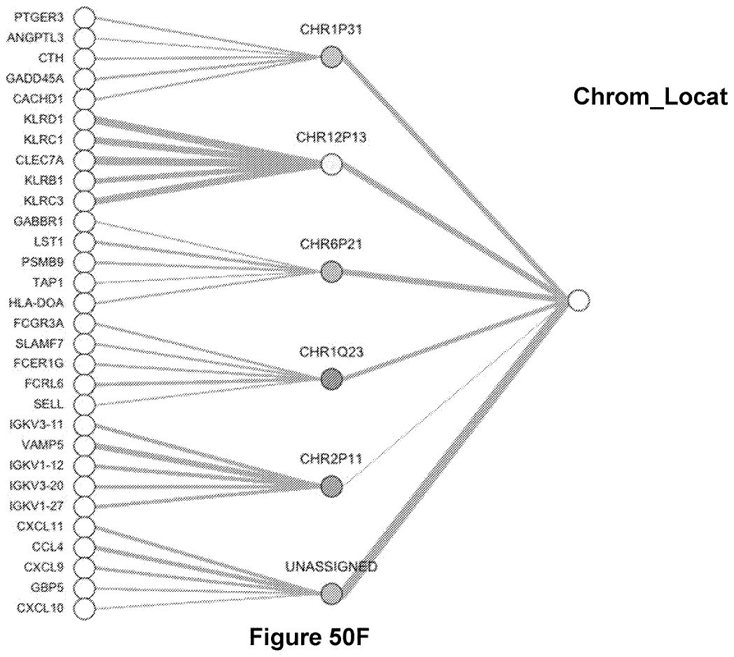

Both the ensemble model and the base learners resulted in high performance, with a ROC AUC in the range 0.93-0.97 on hold-out samples (FIGS. 46A, 50A, 50B, 50C, 50D, 50E, and 50F). In addition to this standard performance metric, the present disclosure defined a score measuring the interpretability of the base neural networks. To this end, the present disclosure used Normalized Enrichment Score (NES), the primary GSEA statistic measuring the association between a gene set and a phenotype of interest. In some embodiments, the systems and methods of the present disclosure quantified the interpretability of a base network as the mean NES of its hidden nodes. To fairly compare NES within and across the different networks, the present disclosure renormalized the NES's by regressing out systematic biases related to gene set size (FIGS. 48 and 49).

The standard performance, combined with the interpretability score, gives a broader evaluation of the base neural networks (FIGS. 46B, 50A, 50B, 50C, 50D, 50E, and 50F). Networks with similar ROC AUC show large variations in the interpretability score. The interpretability of networks derived from pathway annotation libraries was significantly higher than that of regulatory processes (FIGS. 50A, 50B, 50C, 50D, and 50E). This may depend on the larger characteristic gene set size (FIG. 47), which makes the corresponding gene sets less informative. Conversely, Gene Ontology, which contains more specific gene sets, produced more interpretable networks.

In some embodiments, the systems and methods of the present disclosure then examined the base neural network resulting in the best compromise between performance and interpretability (FIG. 46C). The network hidden nodes include various aspects of an immunological response including Interferon Gamma signaling pathway, B cell receptor, cellular defense response, and regulation of T cell activation. Overall, the network clearly separates the two classes (FIG. 46D). By analyzing the network weights, the hidden nodes can be ranked according to their influence on the decision process. The term ‘cellular defense response’ has the largest influence in discriminating between kidney transplant rejection and no rejection. The six input genes in this pathway are consistently up-regulated (FIG. 46E).

Similarly, by analyzing base neural networks derived from other annotation libraries, one can investigate the classification criteria from multiple angles (FIGS. 50A, 50B, 50C, 50D, and 50E), including the involvement of specific transcription factors or preferential genomic locations (FIGS. 50A, 50B, 50C, 50D, and 50E).

### 5.2.4 Interpretable Decisions at the Sample-Level

A frequent limitation of standard classifiers is the inability to explain why a new observation is assigned to a specific class. To address this problem, a solution frequently applied in computer vision was adopted. Using the network weights, each observation was mapped from the original input state (the measured genes) to the activation state of the hidden nodes (gene sets from an annotation library). By construction, the separation of the two classes in the hidden space is much more evident compared with the original input space (FIG. 51A). In the hidden layer, each individual sample can be represented as a vector of activation levels corresponding to the different hidden nodes, and visualized as a parallel coordinate plot (FIG. 51B). Although the two classes of patients are evident, the plot also shows important variation in the hidden state of patients within either class.

For each sample, the neural network returns a probability of that sample being in the positive class or negative class. By looking at the distribution of such probabilities over all samples, one can typically distinguish three types of samples: samples assigned to class 0 with high probability (FIG. 51C, black, left); samples assigned to class 1 with high probability (FIG. 51C, dark grey, right); samples whose decision appears more uncertain (FIG. 51C, light gray middle). To better understand the characteristics of the three groups, the present disclosure generated a corresponding characteristic hidden state. This analysis generates a continuous deformation from profiles of patients predicted to be in class 0 to patients predicted in class 1. New observations can be mapped into this space to reveal what functions and processes make a patient more similar to either group, ultimately driving a certain decision.

In some embodiments, the systems and methods of the present disclosure show that xnnet is instrumental in clarifying and visualizing the decision process for new observations.

### 5.3 Discussion

### 5.3.1 The Present Disclosure Developed a Solution to the Problem of Interpretable Classification.

In some embodiments, the systems and methods of the present disclosure shows analogies with previous works integrating prior knowledge in neural networks. However, a unique feature of our work is the selection of input and hidden nodes that integrates the most established bioinformatics tools for analysis of transcriptional data. This results in small networks capturing the most important genes and gene sets.

### 5.3.2 Integration of Prior Knowledge as a Way to Reduce the Risk of Shortcut Learning.

A fundamental need of classification in biomedical contexts is the ability to explain exactly what drives the decision process. Inspired by related problems in computer vision, the present disclosure addressed this problem by tracking how the activation of the hidden state changes from one class to the other in the training set. Given a new sample, this analysis enables us to identify the components most relevant to the decision process. Techniques from adversarial learning would then make it possible to define minimal changes to the input genes that would cause a change in the decision, which may be useful for robust classification.

An aspect of interpretable models is their dependence on prior knowledge, which is typically incomplete and prone to false positive and false negative associations between genes and gene sets. Xnnet is able to partly overcome this problem by selecting non annotated genes that are very relevant to the classification problem. A similar approach may be adopted for the edges, however this may increase the network complexity and overall interpretability.

### 5.3.3 Discuss Extension to Other Omics Data Types

In biomedical contexts, machine learning classification poses new challenges that make conventional models and performance measures insufficient. By integrating prior knowledge and the most established bioinformatics tools, xnnet provides a new solution to interpretable and explainable classification.

### 5.4 Methods

### 5.4.1 Availability and Pre-Processing of Annotation Libraries

Annotation libraries were downloaded from maayanlab.cloud/Enrichr/#stats. The library size, defined as the number of gene sets contained in each library, is highly variable ranging from 22 to 3340 (FIG. 45). To alleviate potential biases due to differences in library size, libraries with over 1000 gene sets were randomly split into smaller libraries each of which having size<1000.

### 5.4.2 Availability and Pre-Processing of Transcriptional Profiling Data

All datasets used in this work are publicly available from GEO and were used as follows. GSE45291: The classifier was built to distinguish a random subset of 20 out of the available 292 samples from subjects with SLE from the 20 control samples at baseline (time 0). GSE37250: The classifier was built to distinguish the 195 samples from subjects with active tuberculosis from the 167 samples with latent tuberculosis. GSE57338: The classifier was built to distinguish the 82 samples from subjects with idiopathic dilated cardiomyopathy from the 95 samples with ischemic heart disease. GSE36059: The classifier was built to distinguish samples from biopsies of subjects with kidney transplant rejection from subjects with no rejection.

All datasets were pre-processed as follows. Expression data were log-normalized and quantile normalized across arrays. Probe identifiers were mapped to official gene symbols. Multiple probes corresponding to the same gene symbol were collapsed by choosing the probe with maximum coefficient of variation. Finally, the expression data was standardized before being used as input to the neural network classifier.

### 5.4.3 Building and Training Xnnet

The xnnet nodes are selected as a result of established bioinformatics tools to analyze gene expression data. Node selection and network training are performed only on bootstrap samples generated from the training set, which corresponds to 75% of the input dataset. Because functions and regulatory processes are more robust features compared to individual genes, our approach starts by identifying the most significantly enriched differential gene sets scored by GSEA (Subramanian et al. 2005) for each annotation library and bootstrap sample. These gene sets play the role of hidden nodes (typically 3-5) in the network. From each hidden node, the present disclosure rank its member genes by the corresponding fold-change between the two classes, as estimated from differential expression analysis between the classes. Genes with the largest pi-value within each hidden node (typically 3-5 per hidden node) are then selected as the input genes.

As the selection of input nodes is completely driven by significant annotation terms, the above strategy would fail to capture important input nodes that are not currently annotated. To overcome this problem, the present disclosure extend the network to include a hidden node of relevant “unassigned genes”, consisting of top differentially expressed genes that are not selected in the previous steps.

Weights of edges between the selected input and hidden nodes that are not supported by prior knowledge are initialized to zero and excluded from the network training. For each network, the decay parameter is determined through a grid search in the range 0.1-0.5 through bootstrapping. Network training is performed using the caret package in R.

Networks corresponding to different libraries are trained in parallel and used as base learners whose probabilistic outputs are averaged in an ensemble model.

### 5.5 Benchmark

To compare the performance of xnnet with logminer, the present disclosure followed the strategy proposed in (Avey et al. 2017). The benchmark datasets GSE45291, GSE37250, and GSE36059 were randomly split multiple times in two components corresponding to 75% and 25% of the data. For each split, the xnnet was trained on 75% and the resulting performance was measured on the 25%. This strategy produces a distribution of accuracy values that enables robust evaluation of performance. Xnnet has a flexible structure that depends on the number of input and hidden nodes. To determine these parameters, the present disclosure explored a grid search limited to small values of input nodes (3-5) and of hidden nodes (3-5), for a total of 9 combinations. Finally, the present disclosure selected the combination resulting in the maximum median accuracy over the multiple random splits in a training and test set. To guarantee network interpretability, the grid search is constrained to small values of input and hidden nodes (see Methods).

### 5.5.1 Measuring Interpretability

### Interpretability at the Single-Sample Level.

Transcriptomics datasets include tens of thousands of input genes, and annotation libraries contain hundreds of gene sets. Thus, for each annotation library, xnnet returns a sparse neural network whose edges and nodes carry a straightforward interpretation that can be easily interpreted (FIG. 43).

The network nodes are selected in a data-driven manner, in order to capture the most relevant biological signals while minimizing the network complexity.

Part 6: Systems and Methods for Control of Regulation Extracted from Multi-Omics Assays (CREMA)

### Description:

In some embodiments, the disclosed CREMA (Control of Regulation Extracted from Multi-omics Assays) addresses the problem of identifying transcriptional factor-regulatory site-gene regulation units using same cell multiomics data. The disclosed analysis utilizes the full power of same cell multiomics and provides more robust identification of inter-related regulatory changes.

In some embodiments, the disclosed systems and methods provide a wide utility, such as, but not limited to, helping identify targets in developing a chromatin or mRNA diagnostic signature.

In some aspects, provided herein is a computational framework for understanding gene regulation from multi-omics same-cell measurements of both gene expression and chromatin accessibility.

In some embodiments, the disclosed model is advantageous in two aspects: (1) by incorporating chromatin accessibility, it tends to identify direct TF-target relations rather than indirect correlations; and, (2) it identifies regulatory domains in both the proximal and distal regions.

One aspect in accordance with part 6 of the present disclosure provides a method for determining one or more transcription factors that regulate a first gene in a cell type. The method comprises obtaining a single nucleus multi-omics dataset, in electronic form, comprising: (i) a respective ATAC fragment count for each ATAC peak in a corresponding plurality of ATAC peaks, for each respective cell in a plurality of cells, and (ii) a respective discrete attribute value for each gene transcript in a corresponding plurality of gene transcripts, for each respective cell in the plurality of cells, where the plurality of cells is from a biological sample from a subject.

A plurality of transcription factor binding sites is obtained. Each respective transcription factor binding site in the plurality of transcription factor binding sites is associated with (i) a gene in a plurality of genes and (ii) a transcription factor in a plurality of transcription factors.

For each respective cell represented in the plurality of cells, for each respective transcription factor binding site in the plurality of transcription factor binding sites, the respective ATAC fragment count for each corresponding ATAC peak from the respective cell in the single nucleus multi-omics dataset within a threshold distance of the respective transcription factor binding site is used to determine a respective binary openness assignment for the respective transcription factor binding site for the respective cell represented in the plurality of cells.

For each respective cell represented in the plurality of cells, for each respective gene in the plurality of genes, where the plurality of genes includes the first gene, a respective regressor of form:

$$z = f ( {y}_{ij} \cdot {x}_{i} )$$

where z is the respective discrete attribute value of the respective gene for the respective cell in the single nucleus multi-omics dataset, xᵢ is the respective discrete attribute value of the ith transcription factor associated with the respective gene for the respective cell in the single nucleus multi-omics dataset, and yᵢⱼ is the binary openness of the jth transcription factor binding site of the ith transcription factor in the respective cell, ƒ is a linear model, and i and j are positive integers is constructed, thereby forming a plurality of regressors.

The plurality of regressors are regressed against the single nucleus multi-omics dataset, thereby identifying one or more transcription factors in the plurality of transcription factors that regulate the first gene.

In some embodiments, a first transcription factor binding site in the plurality of transcription factor binding sites is associated with a first transcription factor in the plurality of transcription factors when the first transcription factor binding site is within a window around a start site of the first transcription factor. In some such embodiments, the window is +/−50 kilobases, +/−100 kilobases, +/−150 kilobases, or +/−200 kilobases around a start site of the first transcription factor.

In some embodiments the threshold distance is a value between 25 bases and 1000 bases. In some embodiments the threshold distance is 400 bases.

In some embodiments the plurality of cells comprises a plurality of cell types and the method further comprises using the plurality of regressors to identify one or more transcription factors in the plurality of transcription factors that regulate the first gene in a first cell type in the plurality of cell types.

In some embodiments, the plurality of cell types comprises 2, 3, 4, 5, 6, 7, 8, 9, or 10 different cell types.

In some embodiments, the plurality of cells comprises 50 or more cells, 100 or more cells or 1000 or more cells.

In some embodiments, each corresponding plurality of gene transcripts represents 50 or more genes, 100 or more genes, 150 or more genes, 200 or more genes, or 250 or more genes, and each corresponding plurality of ATAC peaks comprises 50 or more peaks, 100 or more peaks, 150 or more peaks, 200 or more peaks, or 250 or more peaks.

In some embodiments, the plurality of genes comprises 2, 3, 4, 5, 6, 7, 8, 9, or 10 genes.

In some embodiments, the plurality of genes comprises 10 or more, 20 or more, or 100 or more genes.

In some embodiments, the plurality of genes consists of between 2 and 15000 genes.

In some embodiments, the plurality of regressors comprises between twenty and one thousand regressors.

In some embodiments, the plurality of regressors comprises 100 or more regressors.

Another aspect of the present disclosure provides a computer system for determining one or more transcription factors that regulate a first gene in a cell type. The computer system comprises one or more processors and memory addressable by the one or more processors. The memory stores at least one program for execution by the one or more processors. The at least one program comprising instructions for performing any of the methods discloses in part 6 of the present disclosure.

Another aspect of the present disclosure provides a non-transitory computer readable storage medium. The non-transitory computer readable storage medium stores instructions, which when executed by a computer system, cause the computer system to perform a method for determining one or more transcription factors that regulate a first gene in a cell type. The method comprises any of the methods disclosed in part 6 of the present disclosure.

### 6.1 Abstract

Single same cell RNAseq/ATACseq multiome data provide unparalleled potential to develop high resolution maps of the cell-type specific transcriptional regulatory circuitry underlying gene expression. In some embodiments, the systems and methods of the present disclosure present a framework that recovers the full cis-regulatory circuitry by modeling gene expression and chromatin activity in individual cells without peak-calling or cell type labeling constraints. In some embodiments, the systems and methods of the present disclosure demonstrate that the disclosed systems and methods overcome the limitations of existing methods that fail to identify about half of functional regulatory elements that are outside the called chromatin “peaks”. These circuit sites outside called peaks are shown to be important cell type specific functional regulatory loci, sufficient to distinguish individual cell types. Analysis of mouse pituitary data identifies a Gata2-circuit for the gonadotrope-enriched disease-associated Pcsk1 gene, which is experimentally validated by reduced gonadotrope expression in a gonadotrope conditional Gata2-knockout model. In some embodiments, the systems and methods of the present disclosure provide a web accessible human immune cell regulatory circuit resource.

Elucidating the mechanisms underlying the regulation of gene expression is important for understanding the molecular basis of cell type identity, biological processes and disease. Cis-gene regulatory circuits, which consist of transcription factors (TFs) and their interactions with specific cis-regulatory sites on chromatin, serve a major role in determining gene expression. RNA-seq and ATAC-seq multiome technology, by profiling the regulatory circuit components within each nucleus, sets the stage for reconstructing cell type-specific gene control circuitry at single cell resolution. See Kim et al., 2009; Ma et al., 2020; Chen et al., 2019, each of which is hereby incorporated by reference in its entirety for all purposes.

Analysis of single cell data typically initially reduces the search space by first calling chromatin peaks in pseudo-bulk data. Studies of ChIP-seq data have shown that weak binding sites, while functionally important, are often missed by genome-wide peak calling methods. It was considered that for single cell ATAC-seq data, the peak calling algorithms also may fail to identify many open or partly open regulatory loci that do not reach the statistical significance required for differential accessibility calling. Evaluation of this possibility using functional domain databases indicated that restricting the circuit search to functional peaks neglects about half of known functional regulatory regions. Accordingly, a framework that does not require peak calling is desirable to leverage the power of single cell multiome datasets for understanding gene control mechanisms. See Stuart et al., 2021; Schep et al., 2017; Bravo Gonzalez-Blas et al., 2019; Nakato et al., 2016; Landt et al., 2012; and Schmidt et al., 2010, each of which is hereby incorporated by reference in its entirety for all purposes.

To address this bottleneck, the systems and methods of the present disclosure developed a framework (e.g., Control of Regulation Extracted from Multiomics Assays) for the systematic survey of gene regulatory circuits from single cell multiomics data. The disclosed framework recovers circuitry by modeling gene expression and chromatin accessibility directly over the entire cis-regulatory region, without being restricted by either peak calling or cell type identification. Improvement of regulatory circuit recovery by the disclosed systems and methods relative to the current state-of-the-art method is shown below as well as the value of the disclosed framework for identifying new circuitry and accessibility site variation that defines individual cell types. Applying the disclosed framework to mouse pituitary data, the systems and methods of the present disclosure show how it can identify cell type specific circuits and identify a gata2-circuit regulating a disease-associated target that is validated in a conditional gata2 mouse knockout model. The framework is available at github.com/zidongzh/CREMA). The systems and methods of the present disclosure use CREMA to generate a web-accessible research resource comprising the regulatory circuitry of human blood immune cells, which is available at rstudio-connect.hpc.mssm.edu/crema-browser/.

### 6.2 Results

### 6.2.1 Motivation

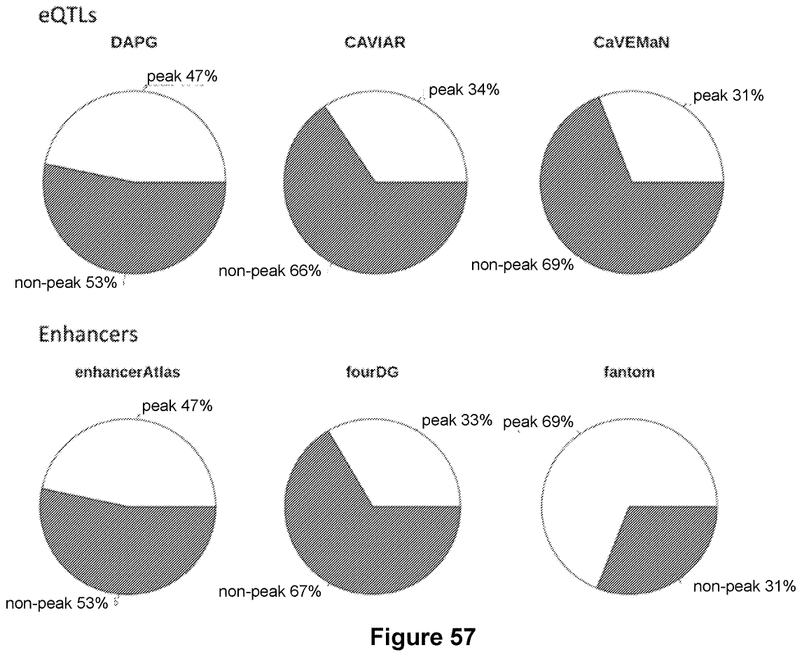

Each gene regulatory circuit consists of a transcription factor (TF), a cis-regulatory domain that interacts with the TF, and a target gene that has altered transcription resulting from this interaction. Multiple circuits involving the same TF binding at different locations or multiple TFs interacting at the same or different loci are the major cis-regulatory mechanisms regulating gene expression. Existing gene control circuit analysis methods only identify the potential regulatory domains for these circuits that are contained within called chromatin peaks in ATAC-seq data. In order to assess the degree to which this restriction may limit identification of cis-regulatory domains and their associated circuits, the fraction of known regulatory loci in human blood that are outside of called chromatin peaks was investigated. The proportion of known functional domains in two reference databases that were contained within called chromatin peaks was determined using high resolution reference single cell ATACseq data (see Online Methods). A majority of eQTLs in the GTEX DAPG fine-mapped eQTL database (See Wen et al., 2017) and of enhancers in the EnhancerAtlas database 2.0 (Gao and Qian, 2019) are located outside of the peaks called using reference high resolution human peripheral blood mononuclear cell (PBMC) chromatin accessibility data (FIG. 53A) (Jiang et al., 2022; Hao et al., 2021). Similar results were observed in other fine-mapped eQTL and enhancer databases (FIG. 57). These results suggest that multiome circuit inference methods that rely on chromatin peak calling will miss about half of the regulatory landscape and circuitry underlying gene control. To address this gap, the systems and methods of the present disclosure developed the disclosed framework to improve the reconstruction of gene regulatory circuitry.

### 6.2.2 Example Framework

An example framework in accordance with the present disclosure was designed to identify transcriptional regulatory circuits over the entire cis-regulatory region of each gene. The disclosed framework finds circuits that are supported by the co-incidence of TF expression, target gene expression and binding site accessibility in individual cells. A schematic of an example of the disclosed method is shown in FIGS. 53B and 53C. The disclosed framework selects the target genes to model that have detectable expression above a threshold in a minimum number of cells and proportion of all cells (See Methods). For each of these target genes, the disclosed framework uses motif analysis to select potential TF binding sites in a +/−100 kb window surrounding the transcription start site (TSS).

Each site, together with the TF and gene constitute a potential regulatory circuit. A linear model for each potential circuit is constructed where the expression of each gene in each cell is a function of the expression of the TF and the binarized accessibility in a 400 bp window centered on the site. Using all the cells in the dataset, the TF-site-gene circuits showing the best fits are selected (See Methods).

### 6.2.3 Benchmarking

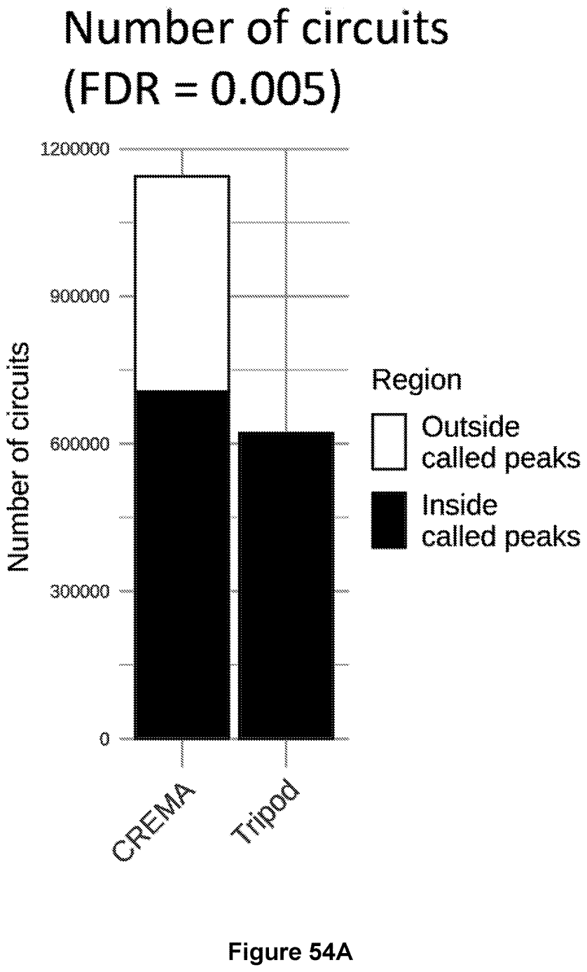

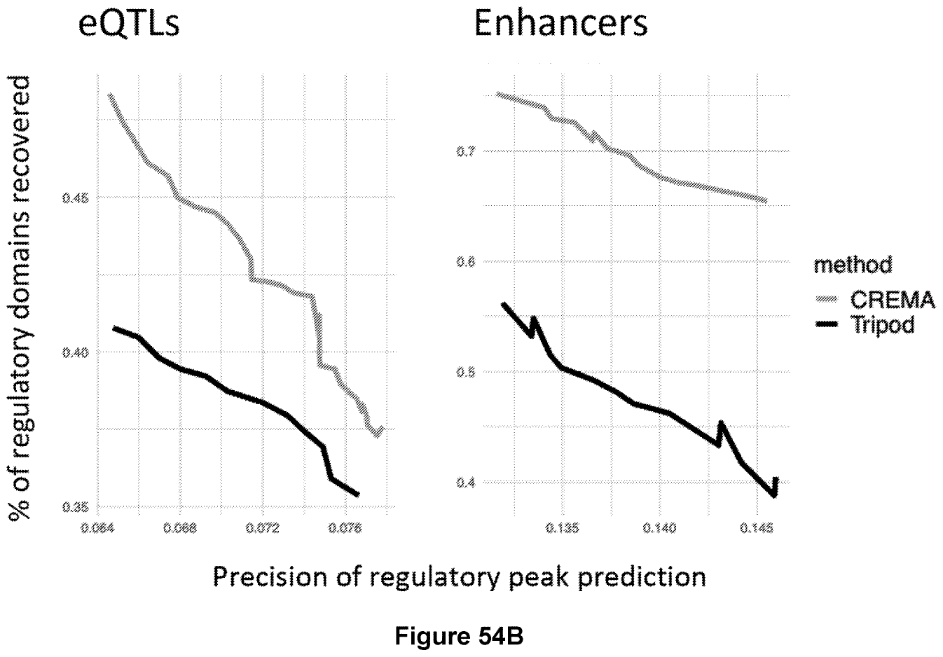

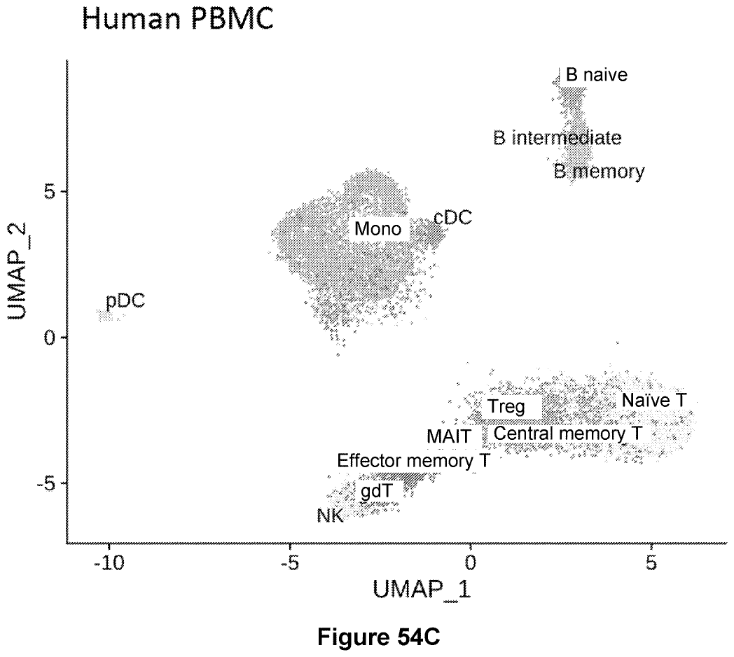

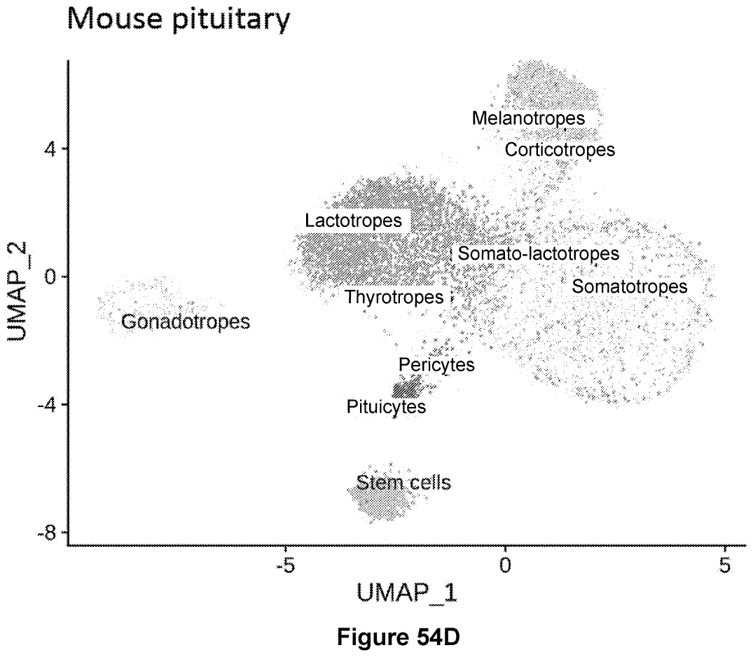

Because the disclosed framework does not rely on a predefined set of chromatin peaks called at the pseudo-bulk level, it has the potential to recover many more regulatory domains compared to analyses relying on peak calling. Analysis of single cell blood multiome data with the disclosed framework identified regulatory circuits both inside and outside of chromatin peaks. The number of circuits identified within peaks was comparable to that obtained using the currently available multiome regulatory circuit discovery method, which relies on peak calling (Jiang et al. 2022). The disclosed framework also identified a large number of circuits that are outside of called peaks, which cannot be found with a peak-calling dependent method (FIG. 54A).

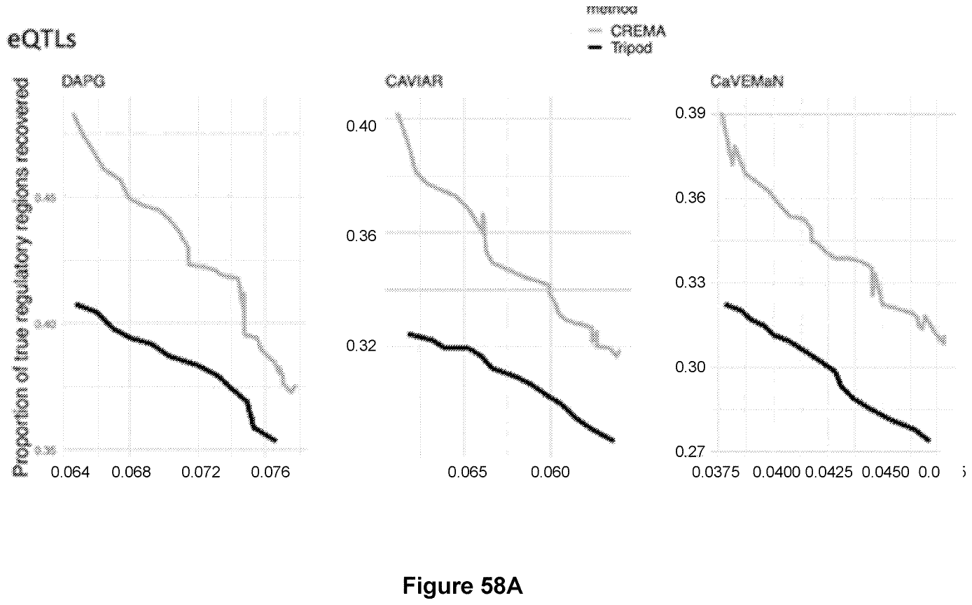

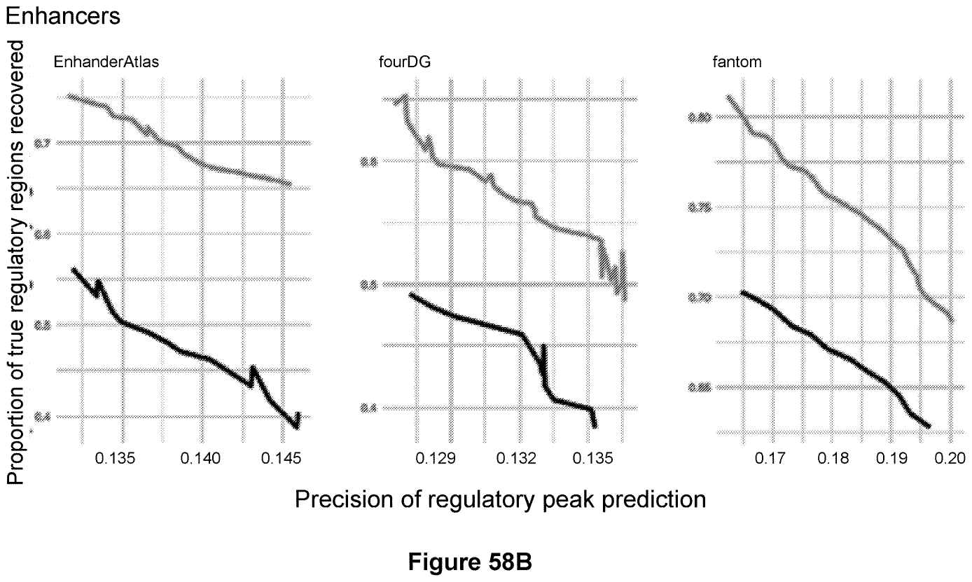

The importance of the additional regulatory landscape recovered by the disclosed framework was evaluated using gold standard functional domain databases. The disclosed framework greatly improved recovery of circuits acting at functional domains in both reference eQTL and enhancer databases (FIG. 54B, and FIGS. 58A and 58B). To further assess the importance of the extra-peak regulatory circuitry that the disclosed framework recovers, it was evaluated whether the regulatory circuit chromatin domains identified by the disclosed framework that were outside of called chromatin peaks contributed to cell type specification. In addition to the PBMC dataset analysis shown in FIG. 54A, a mouse pituitary multiome dataset was generated that was also analyzed using the disclosed systems and methods. In both cases, only the chromatin regulatory sites discovered by the disclosed framework that are outside of called peaks were used as features for UMAP projections. It was found that in both tissues, the major cell types were distinguishable (FIGS. 54C and 54D). These results indicate that the comprehensive circuitry mapping achievable with the disclosed framework elucidates the gene control mechanisms underlying the differences among cell types.

### 6.2.4 Gata2 Circuit Identified Using the Disclosed Framework

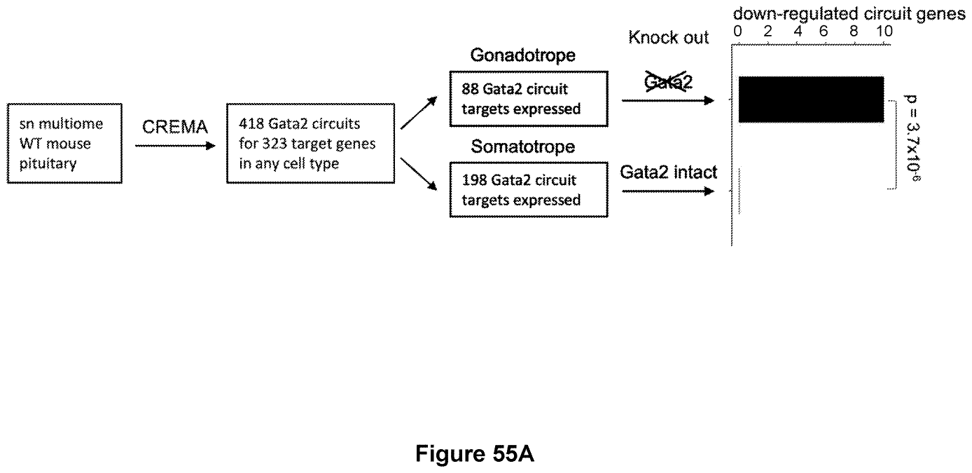

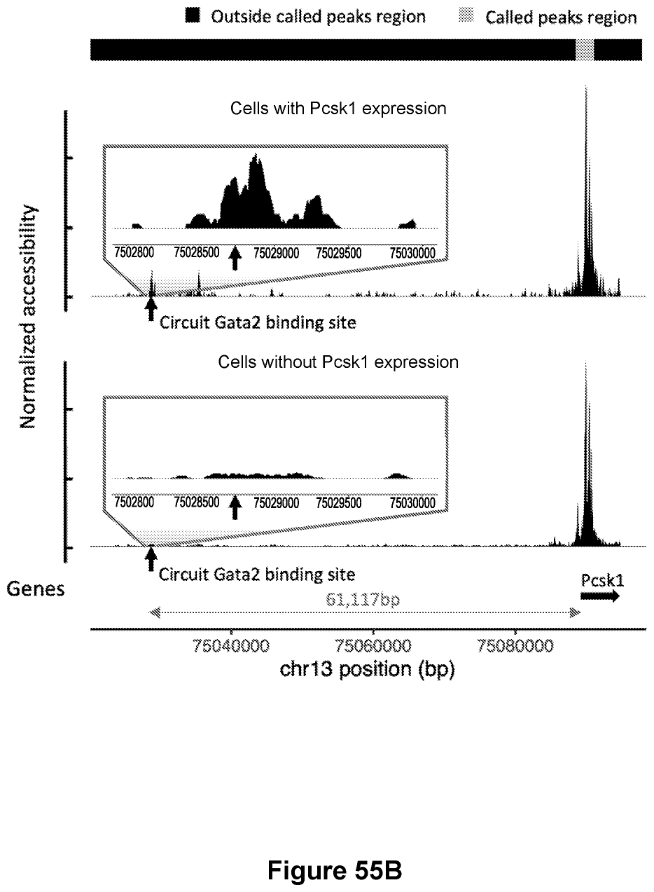

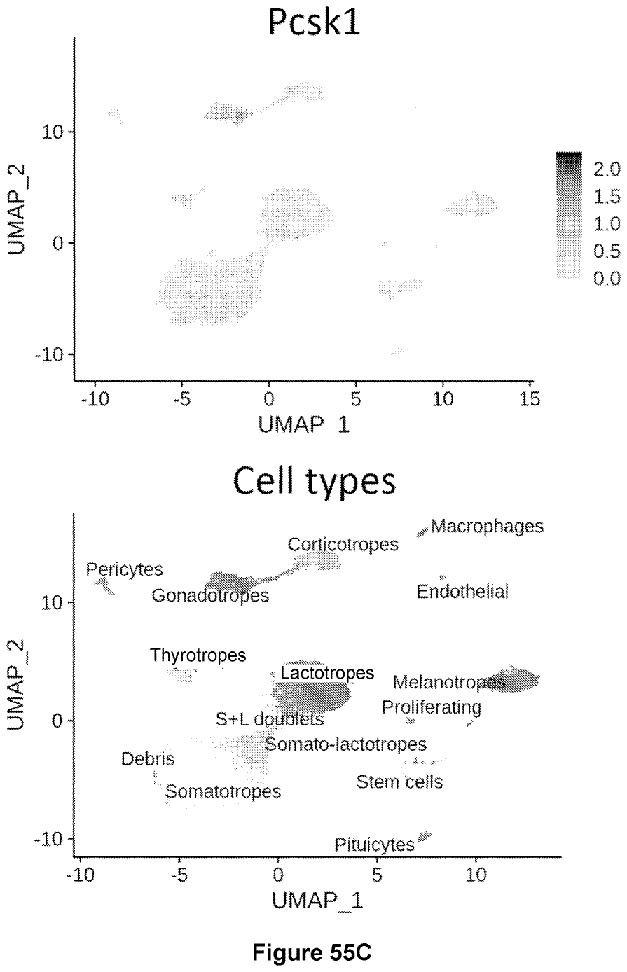

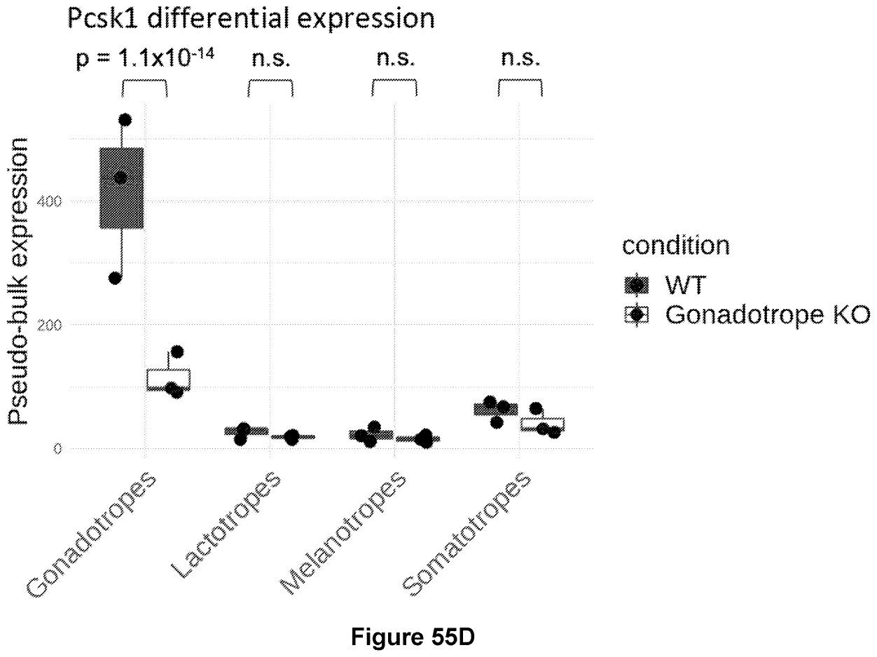

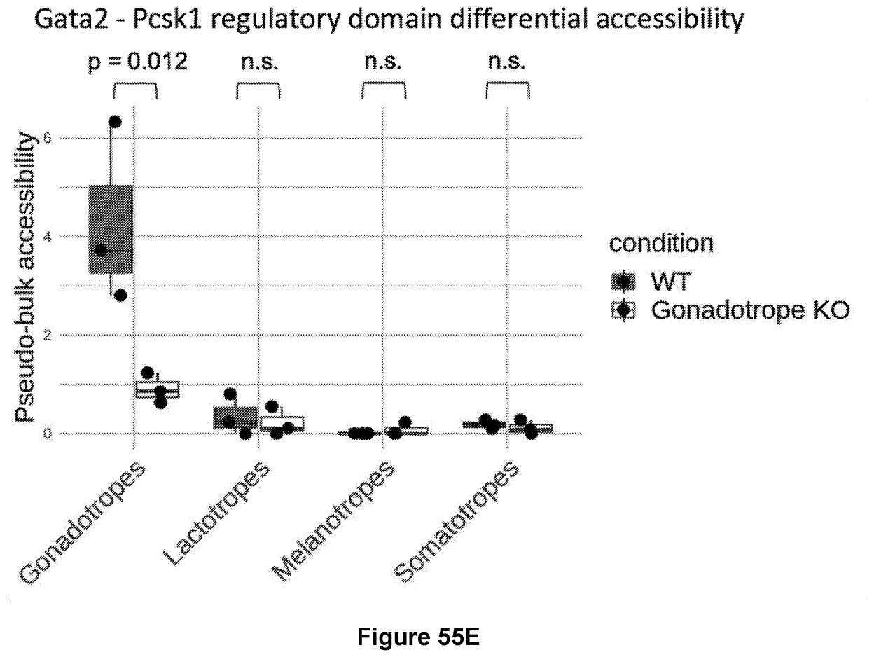

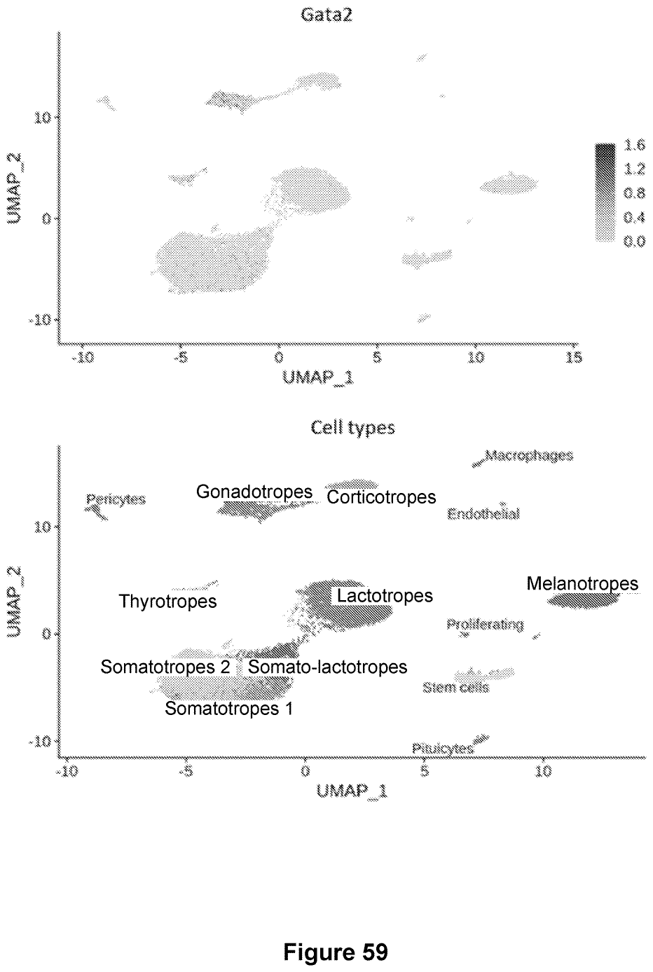

The regulatory circuits identified by the disclosed framework in pituitary involving the pioneer TF, Gata2 (Wu et al., 2014), were studied. In pituitary, Gata2 is necessary for gonadotrope lineage specification and regulates the production of follicle-stimulating hormone. In mouse pituitary single cell multiome data, the disclosed framework identified circuits regulating 323 target genes. Because Gata2 was highly expressed in both gonadotrope and somatrope cell types (FIG. 59), attention was turned to the circuits in these two cell types for validation. Among the 323 target genes in Gata2 circuits, 88 were highly expressed in the gonadotropes and 200 were highly expressed in the somatotropes (FIG. 55A). To validate these circuits predicted by the disclosed framework, the expression of the target genes for these circuits in single cell RNAseq data obtained from a gonadotrope-specific conditional Gata2 knockout (Schang et al., 2022) was assessed. In this knockout, Gata2 function was absent in gonadotropes, and 10 of the predicted gonadotrope Gata2 target genes were significantly down regulated. In contrast, Gata2 function was preserved in somatotropes and none of the predicted Gata2 target genes showed significant down regulation (p=3.5×10-6, z-test of two proportions, FIG. 55A). These results provide strong support for the recovery of the Gata2 circuitry by CREMA.

The Gata2 circuit involved in the regulation of the Pcsk1 gene (See Folon et al., 2023; Frank et al. 2013; and Wei et al. (2014)), which is implicated in infertility, obesity and diabetes. The disclosed framework identified a significant cis-regulatory domain with a Gata2 binding motif at 61 kb upstream of the Pcsk1 TSS. This domain was highly accessible in cells with Pcsk1 expression but was not included within called peak regions and could not have been identified by a peak-calling dependent method (FIG. 55A). Pcsk1 was expressed in multiple cell types in the pituitary: gonadotropes, lactotropes, melanotropes and somatotropes (FIG. 55B). However, the expression of Pcsk1 and the accessibility of this cis-regulatory domain were down-regulated only in the gonadotropes in the conditional knockout data, where Gata2 activity was eliminated, while remaining unchanged in the other cell types (FIG. 55C). These results demonstrate the usefulness of the disclosed framework for leveraging single cell multiome data to obtain insight into the regulatory circuitry controlling gene expression at cell type resolution.

### 6.2.5 Regulatory Circuitry Resource for Human Immune Cells

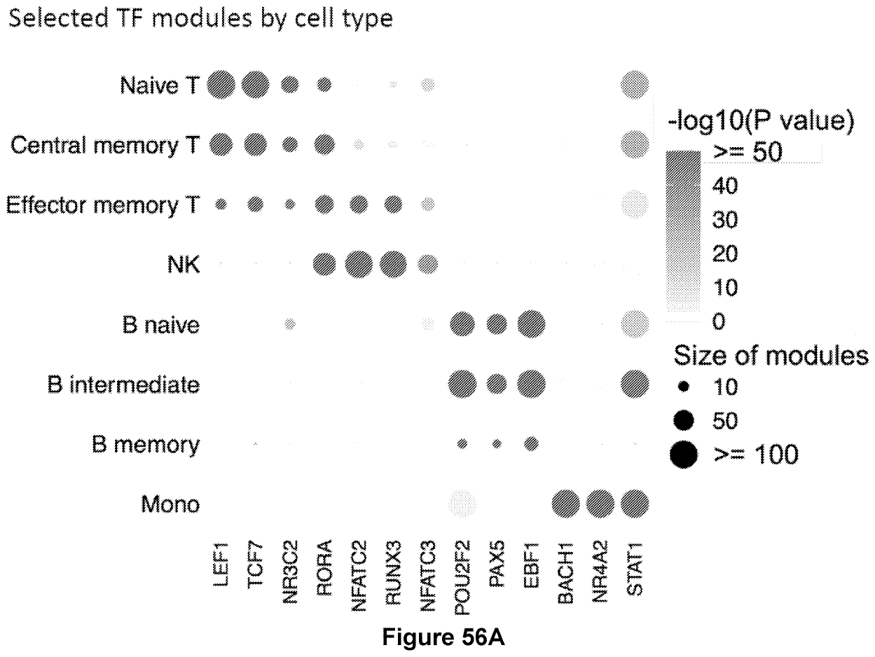

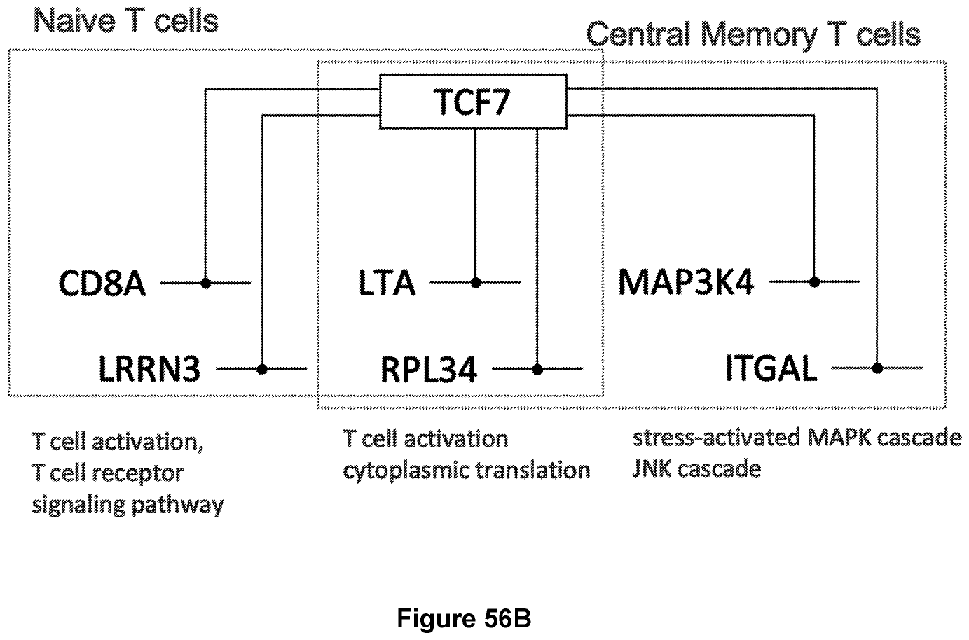

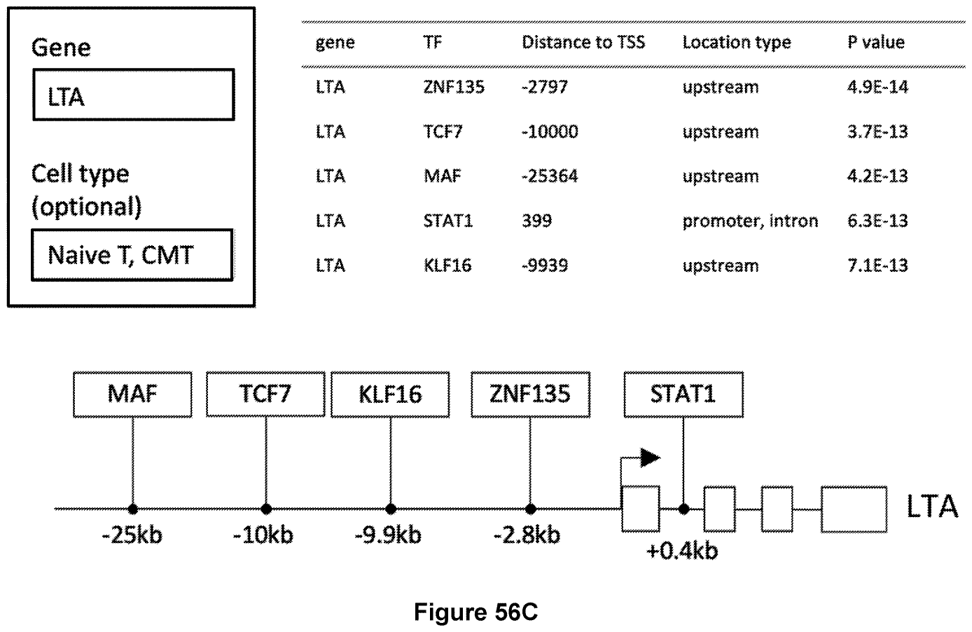

The orchestration of the immune response in health and disease depends on the modulation of gene expression in the different immune cell types. In order to provide a resource for the study of gene regulatory mechanisms in immune cells, the disclosed framework was used to identify the regulatory circuitry in blood using a single cell multi-omic dataset and to provide this analysis as a resource. Circuitry can be summarized both in a TF-centric and gene-centric manner. In some embodiments, the systems and methods of the present disclosure first summarized the regulatory circuits identified by the disclosed framework in a TF-centric perspective, defining a TF module as the collection of regulatory circuits sharing a common TF in each cell type (see Methods). Selected TF modules and their activities in the major immune cell types are presented in FIG. 56A.

As an example, the systems and methods of the present disclosure focused on the TCF7 module, which is active mainly in the naive T cells and central memory T cells. Within the TCF7 module, there were circuits shared by the two cell types, such as the circuit regulating the target gene LTA which encodes a cytokine expressed by resting and activated T cells (FIG. 56B) (Ware et al. 1992; and Ohshima et al. 1999). There were also TCF7 circuits specifically active in one of the cell types. For example, the TCF7-CD8A circuit was active only in the naive T cells and CD8A is involved in T cell activation. The TCF7-MAP3K4 circuit was active only in the central memory T cells and MAP3K4 is involved in the stress-response MAPK cascade (FIG. 56B and Table 6.1).

TABLE 6.1
List of TCF7 target genes identified by CREMA in the immune cell types

| Cell Type | Genes |
|---|---|
| Central memory T | CDC14A, TNFRSF1B, NBPF15, S100A11, FLAD1, ITGB1, |
| | ARID5B, CD5, ETV6, DUSP16, PLEKHA5, ST8SIA1, KIF21A, |
| | IFNG-AS1, KLRB1, EPSTII, LCP1, LPAR6, KLF12, LINC00402, |
| | ITGAL, MAF, ABR, RARA, CD226, IL411, MYOIF, DPP4, |
| | ICOS, EPHA4, WDFY1, FOSL2, GALM, EML4, CFAP36, |
| | RRBP1, TSHZ2, TMX4, MICAL3, PARVB, TIGIT, ARHGAP31, |
| | MB21D2, KAT2B, CMTM8, CMTM6, CCR4, CLDND1, TRIM2, |
| | ARAP2, ANTXR2, PTPN13, GPRIN3, ADAM19, SEMA5A, |
| | MAP3K5, TNFAIP3, ZC3H12D, MAP3K4, HLA-DQB1, |
| | TBXAS1, NCALD, BLK, MTSS1, RAB11FIP1, AP3M2, GLIPR2, |
| | ANXA1, LINC00892, CD40LG, FAAH2, CXCR3 |
| Naive T | SELL, ITPKB, MDS2, LDLRAP1, MANIC1, LRRC7, CA6, |
| | RGS10, SFMBT2, CRTAM, PDE3B, PSMA1, PGGHG, KLRK1, |
| | KLRC4, NELL2, KRT72, NAA16, SLC7A8, ACTNI, |
| | LINC01550, APBA2, IGFIR, CCR7, NOG, PITPNC1, SDK2, |
| | CD7, FCGBP, ARRDC5, ZNF331, ITGA6, PDK1, FAM117B, |
| | SNED1, TRABD2A, CD8B, CD8A, SNTG2, PDE9A, PRMT2, |
| | PIK3IP1, OXNAD1, SATB1, SATB1-AS1, TGFBR2, FHIT, |
| | FOXP1, LEF1-AS1, LEF1, MAML3, ATP8A1, RAPGEF6, |
| | NDFIP1, ARHGAP26, IL6ST, RASGRF2, SLC16A10, THEMIS, |
| | PTPRK, CARMIL1, LY86, NT5E, BACH2, NRCAM, LRRN3, |
| | AOAH, LEPROTL1, FAM102A, MLLT3 |
| Both | NOTCH2, MCL1, CTSS, GABPB2, THEM4, RPS27, TPM3, |
| | SMG5, USF1, SDHC, CD247, CACYBP, PTPRC, TRAF3IP3, |
| | RPL11, NIPAL3, RCAN3, WASF2, LCK, RPS8, ZSWIM5, |
| | ECHDC2, PATJ, RPL5, EVI5, ADD3, USP6NL, NMT2, CCDC7, |
| | GDI2, PRKCQ, PRKCQ-AS1, PSAP, SPOCK2, ANAPC16, |
| | RPS24, TMEM123, CD3D, CD3E, CD3G, RPS25, ETS1, |
| | NAPIL4, CD6, FTH1, FAU, MALATI, PACS1, GSTP1, RPS3, |
| | SESN3, BTBD11, RBM19, RPLPO, NCOR2, YARS2, PCEDIB- |
| | AS1, TESPA1, DGKA, RPL41, NACA, GNS, HELB, LYZ, |
| | SLC2A3, DUSP6, BTG1, LINC01619, CLEC2D, TPP2, RASA3, |
| | MRPS31, RGCC, TPT1, EVL, BAZIA, RPS29, KIAA0586, |
| | FUT8, SPTLC2, CALM1, CCDC88C, TC2N, BCL11B, TARSL2, |
| | B2M, MYEF2, USP3, RPLP1, AKAP13, ZNF710, CRTC3, CHD2, |
| | TMEM204, RPS15A, RPS2, ARHGAP17, LAT, CORO7, CYLD, |
| | RBL2, SLC7A6, WWP2, CYBA, RPL13, RPL23A, SLFN5, |
| | MLLT6, RPL23, SKAP1, ABI3, FAM117A, TSPOAP1-AS1, |
| | ACAP1, GRB2, RNF157, SEC14L1, TMC8, CHD3, RNF213, |
| | VAMP2, CSNKID, LDLRAD4, RNF125, BCL2, TMX3, |
| | ARHGAP45, PRDX2, KLF2, RPL18A, IFI30, MOB3A, |
| | KIAA0355, ZNF529, RPS16, EEF2, CEACAM21, POU2F2, |
| | FOSB, RPL18, FLT3LG, NOSIP, RPL13A, RPS11, AP2A1, |
| | ZNF836, RPS9, RPL36, RPS5, CLPP, ALKBH7, STXBP2, CFD, |
| | NCK2, UXS1, GYPC, BIN1, CXCR4, ARHGAP15, RBMS1, |
| | PRKRA, STK17B, AC013264.1, IKZF2, LBH, ZFP36L2, RHOQ, |
| | RPS27A, CCDC88A, CCT4, PPP3R1, AAK1, TET3, MAL, |
| | ADAM17, ZAP70, MGAT4A, SDCBP2-AS1, RBL1, SLC23A2, |
| | TMEM230, GPCPD1, CASS4, ZNF831, RPS21, OGFR, |
| | LINC00649, AGPAT3, TRIOBP, RPL3, GRAP2, ST13, SENP7, |
| | RPL24, RPL32, TNIK, FNDC3B, ACAP2, RPL15, SFMBT1, |
| | CCDC66, RPL34, ZNF827, FBXL5, DDX60L, LAP3, RHOH, |
| | AFAP1, ST8SIA4, CAMK4, TNFAIP8, CDC42SE2, AFF4, SKP1, |
| | IK, ANKH, RPS14, CD74, ITK, CYFIP2, NPM1, SFXN1, |
| | MAML1, RACK1, IL7R, ANKRD55, ZSWIM6, SERINC5, |
| | RPS23, SCML4, STX7, HIVEP2, UTRN, ACAT2, IGF2R, |
| | ERMARD, LTA, LST1, HLA-DRB5, HLA-DQA1, HLA-DRB1, |
| | HLA-DPB1, RPS18, PHF1, SYNGAP1, RPS10, RPL10A, STK38, |
| | CCND3, EEF1A1, SNHG5, PILRA, ZCWPW1, CUX1, NAMPT, |
| | TMEM106B, KDM7A, BRAF, TRBC2, TRBC1, EPHB6, |
| | GIMAP7, STK17A, IKZF1, FGL2, GSAP, SAMD12, MYC, |
| | ST3GAL1, EEFID, ASAHI, CHMP7, PTK2B, SARAF, CEBPD, |
| | AGPAT5, TNFSF8, RPL35, RPL12, PSIP1, DOCK8, RPL36A, |
| | RPL39, RPL10, CFP, PIM2, PPPIR3F, RPS4X |

A full picture of the gene control within each cell type is obtained by aggregating the multiple regulatory circuits involved in the control of specific genes. In the immune cell resource, access to the entire regulatory circuitry was provided within each cell type. The user can query a gene of interest to obtain a list of regulatory circuits targeting this gene, including the TF and the locations of the cis-regulatory domains interacting with these TFs. An example of a query gene LTA and the top five regulatory circuits identified by the disclosed framework is illustrated FIG. 56C. This immune cell resource is designed to help the research community generate hypotheses about the gene control mechanisms specific to immune cell subtypes and may also help the selection of specific TFs to target for therapeutic immune modulation.

### 6.3 Discussion

The disclosed framework leverages single cell multiome data to infer cis-regulatory circuitry covering the entire cis-regulatory region. The disclosed framework identifies cis-regulatory domains by directly combining the local chromatin accessibility of potential TF binding sites and TF expression without relying on calling ATAC peaks. This expanded search space enables the identification of the large proportion of regulatory circuitry outside of called peaks that contributes to gene control and to cell type specification. The performance of the disclosed framework has been validated using public functional domain databases and a conditional knockout model and an immune cell gene circuitry analysis has been developed as a public resource.

For circuits that are located within peaks, because the disclosed framework models local chromatin accessibility of the TF binding site in a small chromatin window relative to the peak region, the disclosed framework provides higher resolution of the chromatin domain for the circuit than peak-calling dependent approaches. In addition to being chromatin peak-agnostic, the disclosed framework provides is cell type agnostic. Cell type identification is utilized only after the disclosed framework provides analysis in order to evaluate the cell type specificity of the circuits identified. This gives the disclosed framework provides the potential to identify circuits in poorly represented or unlabeled cell types or unlabeled cell types.

In some embodiments, the systems and methods of the present disclosure have developed a resource of the full regulatory circuitry of human immune cells to facilitate hypothesis generation and experiment design for the immune research community (rstudio-connect.hpc.mssm.edu/crema-browser/). CREMA, publicly available via an R package (github.com/zidongzh/CREMA), can help realize the potential of multiome datasets to resolve the circuitry underlying gene control in individual cells.

### 6.4 Methods

### 6.4.1 CREMA Framework

### 6.4.1.1 Gene Filtering

In some embodiments, the systems and methods of the present disclosure focused on modeling genes and TFs above a certain level of expression in the dataset. Specifically, the systems and methods of the present disclosure applied 2 filters on the genes: 1) the gene counts must be non-zero in at least 0.1% of the cells or 3 cells, whichever was larger, and 2) the gene total count in all cells should be larger than (0.2%× total cell number).

### 6.4.1.2 Candidate Regulatory Domain Selection

For each target gene, the entire+/−100 kb window around the transcription start site (TSS) was analyzed without reference to ATAC-seq peak calling. In some embodiments, the systems and methods of the present disclosure scanned for potential TF binding sites in this region by motif analysis. The human TF position weight matrices from the JASPAR database and mouse TF position weight matrices from the CIS-BP database were used. For the motif analysis the systems and methods of the present disclosure used the function matchMotifs from the r package motifmatchr having p<5e-5.

### 6.4.1.3 Model Building

To select regulatory circuits supported by the co-incidence of TF expression, target gene expression and binding site accessibility, a linear regression framework was used where the level of TF is weighted by the accessibility of that TF's binding site. Specifically, for each TF and each binding site found in the candidate regulatory domains, the number of ATACseq cut sites overlapping with a 400 bp window centered around the binding site in each single cell was counted, and binarized the results as open (counts>=1) or closed (counts=0). Then the level of TF RNA and the accessibility of TF binding sites were combined in a linear regression:

$$z = f ( {y}_{ij} \cdot {x}_{i} )$$

where z is the RNA level of the target gene, xᵢ is the RNA level of the ith TF, and yᵢⱼ is the binarized chromatin openness of the jth binding site of the ith TF in the candidate regulatory regions, and f is a linear model. The RNA levels used in the model are normalized RNA levels with SCTranscform. The rationale was that TFs with a closed binding site would not be selected as significant regulators in this framework. Because many TFs had more than one binding site, there was high collinearity among the regressors. Therefore, the significance of each TF-site combination was evaluated by linear regression individually and all significant TF-site combinations were reported, instead of using a multi-regression framework.

### 6.4.2 Data and Preprocessing

6.4.2.1 Human PBMC Data from 10× Genomics

The single nucleus multi-omics dataset of human PBMC was provided by 10× Genomics as a reference dataset. Specifically, the dataset “pbmc_granulocyte_sorted_10k” processed using CellRanger v1.0.0 was downloaded from 10× Genomics, and it was processed following the vignette “Joint RNA and ATAC analysis: 10× multiomic” from the R package Signac v1.5.0.

### 6.4.2.2 Single Nucleus Multiome (RNA+ATAC) of Male Mouse Pituitary

The pituitary used in this study was collected from a male C57BL/6 mice aged 10 weeks. Animals were on a 12-hour on, 12-hour off light cycle (lights on at 7 AM; off at 7 PM). Once collected, the pituitary was immediately snap-frozen following dissection, and stored at −80 C until the assay was started.

Nuclei isolation was performed as described in See Ruf-Zamojski et al., 2021; Mendelev et al., 2022. Briefly, the snap-frozen pituitary was thawed on ice. RNAse inhibitor (NEB M0314L) was added to the homogenization buffer (0.32 M sucrose, 1 mM EDTA, 10 mM Tris-HCl, pH 7.4, 5 mM CaCl₂), 3 mM Mg(Ac)2, 0.1% IGEPAL CA-630), 50% OptiPrep (Stock is 60% Media from Sigma; cat #D1556), 35% OptiPrep and 30% OptiPrep right before isolation. The pituitary was homogenized in a dounce glass homogenizer (1 ml, VWR cat #71000-514), and the homogenate filtered through a 40 m cell strainer. An equal volume of 50% OptiPrep was added, and the gradient centrifuged (SW41 rotor at 9200 rpm; 4 C; 25 min). Nuclei were collected from the interphase, washed, resuspended in 1× nuclei dilution buffer (10× Genomics), and counted (Nexcelom K2 counter), each of which is hereby incorporated by reference in its entirety for all purposes.

Sn multiome was performed following the Chromium Single Cell Multiome ATAC and Gene Expression Reagent Kits V1 User Guide (10× Genomics, Pleasanton, CA) on a male mouse wild-type sample. Nuclei were counted as described above, transposition was performed in 10 l at 37 C for 60 min targeting 10,000 nuclei, before loading of the Chromium Chip J (PN-2000264) for GEM generation and barcoding. Following post-GEM cleanup, the library was pre-amplified by PCR, after which the sample was split into three parts: one part for generating the snRNAseq library, one part for the snATACseq library, and the rest was kept at −20 C. SnATAC and snRNA libraries were indexed for multiplexing (Chromium i7 Sample Index N, Set A kit PN-3000262, and Chromium i7 Sample Index TT, Set A kit PN-3000431 respectively). The library was quantified by Qubit 3 fluorometer (Invitrogen) and quality was assessed by Bioanalyzer (Agilent). This library was sequenced first in a Miseq (Illumina) to assess the reads and balance the sequencing pool, then it was sequenced in a Novaseq 6000 (Illumina) at the New York Genome Center (NYGC) following 10× Genomics recommendations.

The sequencing data was preprocessed with cellranger-arc-2.0.0. The dataset was then processed as described by the vignette “Joint RNA and ATAC analysis: 10× multiomic” from the r package Signac v1.5.0. Cell types were identified by label transfer from a well annotated single nucleus RNAseq dataset (Ruf-Zamojski et al., 2021) using the r package Seurat v4.1.0

### 6.4.2.3 Single Nucleus RNAseq and ATACseq of WT and Gata2KO Mice

Processed single nucleus RNAseq and single nucleus ATACseq datasets of 3 wild type mice (WT) and 3 mice with Gata2 conditionally knocked out in the gonadotrope cells of the pituitary were provided by Daniel Bernard's lab at McGill University (Schang et al., 2022). Cell clusters corresponding to the gonadotropes were located using marker genes of gonadotropes as described before.

### 6.4.3 Benchmarking

### 6.4.3.1 Number of Discoveries

Both the disclosed framework and TRIPOD were run on a human PBMC sn multiome dataset. The same FDR cutoff of 0.005 was used for both methods. For TRIPOD, all the TF-peak-gene combinations passing the FDR cutoff were selected and each of these combinations was counted as one regulatory circuit. For the disclosed framework all the TF-site-gene combinations passing the FDR cutoff were selected, and the site locations were overlaid to chromatin peaks to determine whether the regulatory circuit was within peak regions or outside of peak regions.

### 6.4.3.2 Public Databases of True Regulatory Regions

EnhancerAtlas was downloaded from EnhancerAtlas 2.0 database and all the enhancer-gene interactions in blood cell types were combined. Fantom and 4D genome databases were downloaded from the processed datasets provided by the TRIPOD package. Fine-mapped eQTLs were downloaded from GTEx v8. See Table 6.2 for the URLs of these databases.

TABLE 6.2

| Datasets | URL or GEO access number |
|---|---|
| Publicly available | |
| datasets | |
| Single nucleus multiome | https://www.10xgenomics.com/resources/datasets/pbmcfrom-a- |
| of human PBMC | healthy-donor-granulocytes-removed-through-cell-sorting-10- |
| | k-1-standard-1-0-0 |
| Single nucleus RNAseq | GSE190066 |
| and ATACseq of mouse | |
| pituitary Gata2 | |
| conditional knockout | |
| Gold standard databases | |
| for evaluation | |
| DAPG fine-mapped | https://storage.googleapis.com/gtex_analysis_v8/single_tissue_ |
| eQTLs (from GTEx v8) | qtl_data/GTEx_v8_finemapping_DAPG.tar |
| CAVIAR fine-mapped | https://storage.googleapis.com/gtex_analysis_v8/single_tissue_ |
| eQTLs (from GTEx v8) | qtl_data/GTEx_v8_fineapping_CAVIAR.tar |
| CaVEMaN fine-mapped | https://storage.googleapis.com/gtex_analysis_v8/single_tissue_ |
| eQTLs (from GTEx v8) | qtl_data/GTEx_v8_finemapping_CaVEMaN.tar |
| EnhancerAtlas v2.0 | http://www.enhanceratlas.org/downloadv2.php |
| 4D Genome | https://github.com/yuchaojiang/TRIPOD |
| Fantom v5 | https://github.com/yuchaojiang/TRIPOD |
| New datasets | |
| Mouse pituitary single | GSE234943 |
| nucleus multiome | |

### 6.4.3.3 Recovery of True Regulatory Regions

Both the disclosed framework and TRIPOD were applied to the human PBMC sn multiome dataset to extract regulatory regions for the top 1000 variable genes. Specifically, TRIPOD was run with default settings and all regulatory peaks with both level 1 and level 2 testings were extracted. Three enhancer databases and three fine-mapped eQTL databases described in the last section were used to evaluate the precision of regulatory region predictions and recovery of the true regulatory regions. To compare across the two methods, the performance from the two methods was evaluated by setting different FDR cutoffs in the range of 0.1 to 0.0001. For each FDR cutoff, there was calculated: 1) the recovery of true regulatory regions, defined as the percentage of true regulatory regions from the databases that overlap with the regulatory regions predicted by TRIPOD and the disclosed framework, 2) precision of predictions, defined as the percentage of predicted regions that overlap with true regulatory regions from the databases.

Specifically, chromatin peaks predicted by TRIPOD are larger in sizes than the regulatory sites predicted by the disclosed framework, and larger regions are more likely to overlap with a true regulatory region from the reference databases. To make the calculation of the precision of prediction in the same space for TRIPOD and the disclosed framework, the regulatory sites predicted by CREMA were converted to the chromatin peaks that overlapped with these sites for calculating the precision of predictions. If a chromatin peak overlapped with multiple sites from the disclosed framework, the minimum p-value among these sites was used as the p-value for this peak.

### 6.4.3.4 Recovery of Cell Types

The disclosed framework was applied on the human PBMC sn multiome dataset and the mouse pituitary sn multiome dataset. In both cases, regulatory circuits were extracted with an FDR cutoff of 0.0001 and cis-regulatory regions outside of the called chromatin peaks were selected. The chromatin accessibility in these regions was calculated and used as features for LSI and UMAP dimension reduction on the datasets. In the UMAP visualization, the cells were colored by the original cell type annotations obtained by label transfer from reference datasets as described in the “Data and preprocessing” section.

### 6.4.4 Gata2 Regulatory Circuits in the Pituitary

### 6.4.4.1 Extraction of Gata2 Circuits in the Pituitary Cell Types

The disclosed framework was applied to the sn multiome dataset of wildtype mouse pituitary. All the regulatory circuits with an FDR cutoff of 0.0001 were extracted. All the regulatory circuits where Gata2 was the TF were selected. In this dataset, there were 866 gonadotrope cells and 7420 somatotrope cells. For gonadotropes, target genes of Gata2 were determined as active in gonadotropes if they were detected in at least 260 cells (30%) of the gonadotropes. The same cutoff of 260 cells was used to determine Gata2 targets as active in the somatotropes. The number of cells detected was chosen as the cutoff in order to accommodate possible higher heterogeneity within the somatotrope cells. The cell type specific target genes were analyzed for differential expression between the wild type and conditional knockout datasets.

### 6.4.4.2 Differential Analysis of the Gata2-Pcsk1 Circuit

The expression of Pcsk1 and the accessibility of the Gata2 cis-regulatory site chr13:75028714-75028724 was compared between the 3 wild type samples and 3 conditional knockout samples by pseudobulk analysis. The single cell expression and accessibilities were summed at cell type resolution and differential analysis were performed using DESeq2.

### 6.4.5 Regulatory Circuitry of the Human Immune Cells

### 6.4.5.1 Regulatory Circuits in PBMC

The disclosed framework was applied to the sn multiome dataset of human PBMC. Regulatory circuits with a FDR cutoff of 0.0001 were selected.

### 6.4.5.2 Circuit Activities and TF Module Activities in Cell Types

For visualizing the highly active TF modules in the major immune cell types, the circuit activities and TF modules activities were calculated in each cell type. The activity of each regulatory circuit in each cell was calculated by taking the product of the expression level of the TF, the expression level of the target gene and the binarized accessibility of the cis-regulatory site in the cell. To summarize the activities of regulatory circuits at cell type resolution, two methods were used: 1) a binary activity score where a regulatory circuit was defined as active in a cell type if it was active in more than 10% of the cells in that cell type and more than 50 cells of that cell type, 2) a continuous activity score where the activity of a regulatory circuit in a cell type was defined as the proportion of cells in that cell type where the regulatory circuit was active. To summarize the activities of regulatory circuits in a TF-centric view, a TF module was defined as the collection of all the regulatory circuits involving that TF. For each TF module and each cell type, there was calculated 1) the number of active regulatory circuits in that cell type as measured by the binary activity score under that TF module, 2) the specificity of the regulatory circuits of that TF module to that cell type, measured by summing the continuous activity scores of the regulatory circuits and converting to a z score.

### 6.5 Data and Code Availability

The lab generated single nucleus multiome dataset of mouse pituitary is accessible at GSE234943. CREMA is available as an R package at github.com/zidongzh/CREMA. The web-accessible resource of the regulatory circuitry of human blood immune cells is available at rstudio-connect.hpc.mssm.edu/crema-browser/. The source code for the analysis in this manuscript is available at github.com/zidongzh/CREMA_manuscript.

### Part 7: Systems and Methods for Using Latent Variable Decomposition for Predictive Model Development

### Description:

In one aspect, the present disclosure provides systems and methods for using a previously described method, PLIER, to reduce the feature space and improve model development. In some aspects, the present disclosure provides a predictive machine learning model. In some embodiments, the data is reduced to latent variables (LVs) using PLIER which incorporates outside prior information, such as pathways. In some embodiments, specific set of informative LVs are selected. In some embodiments, a machine learning (ML) model is trained.

### REFERENCES FOR PART 1

Schoenfelder and Fraser (2019). Long-range enhancer-promoter contacts in gene expression control. Nat. Rev. Genet. 20, 437-455.
Kim et al., (2009) Transcriptional regulatory circuits: predicting numbers from alphabets. Science 325, 429-432.
modENCODE Consortium et al. (2010) Identification of functional elements and regulatory circuits by Drosophila modENCODE. Science 330, 1787-1797.
Marbach et al. (2016) Tissue-specific regulatory circuits reveal variable modular perturbations across complex diseases. Nat. Methods 13, 366-370.
Wilk et al. (2021) Multi-omic profiling reveals widespread dysregulation of innate immunity and hematopoiesis in COVID-19. J. Exp. Med. 218, e20210582.
Krijger and de Laat (2016). Regulation of disease-associated gene expression in the 3D genome. Nat. Rev. Mol. Cell Biol. 17, 771-782.
Cao et al. (2018) Joint profiling of chromatin accessibility and gene expression in thousands of single cells. Science 361, 1380-1385.
Kreitmaier et al. (2022), Insights from multi-omics integration in complex disease primary tissues. Trends Genet. 39, 46-58.
Stuart et al. (2019) Comprehensive integration of single-cell data. Cell 177, 1888-1902.
Ma et al. (2020) Chromatin potential identified by shared single-cell profiling of RNA and chromatin. Cell 183, 1103-1116.
Jiang et al. (2022) Nonparametric single-cell multiomic characterization of trio relationships between transcription factors, target genes, and cis-regulatory regions. Cell Syst. 13, 737-751.
Cao and Gao (2022) Multi-omics single-cell data integration and regulatory inference with graph-linked embedding. Nat. Biotechnol. 40, 1458-1466.
Javierre, et al. (2016) Lineage-specific genome architecture links enhancers and non-coding disease variants to target gene promoters. Cell 167, 1369-1384.
Arnold et al (2006). Changing patterns of acute hematogenous osteomyelitis and septic arthritis: emergence of community-associated methicillin-resistant Staphylococcus aureus. J. Pediatr. Orthop. 26, 703-708.
Saavedra-Lozano et al. (2008) Changing trends in acute osteomyelitis in children: impact of methicillin-resistant Staphylococcus aureus infections. J. Pediatr. Orthop. 28, 569-575.
Liao et al. (2003) Network component analysis: reconstruction of regulatory signals in biological systems. Proc. Natl Acad. Sci. USA 100, 15522-15527.
Tran et al. (2005). gNCA: a framework for determining transcription factor activity based on transcriptome: identifiability and numerical implementation. Metab. Eng. 7, 128-141.
Kartha et al. (2022) Functional inference of gene regulation using single-cell multi-omics. Cell Genom. 2, 100166.
Teng et al. (2015) 4DGenome: a comprehensive database of chromatin interactions. Bioinformatics 31, 2560-2564.
Mumbach et al. (2017) Enhancer connectome in primary human cells identifies target genes of disease-associated DNA elements. Nat. Genet. 49, 1602-1612.
Arunachalam et al. (2020) Systems biological assessment of immunity to mild versus severe COVID-19 infection in humans. Science 369, 1210-1220.
Lucas et al. (2020) Longitudinal analyses reveal immunological misfiring in severe COVID-19. Nature 584, 463-469.
Mathew et al. (2020) Deep immune profiling of COVID-19 patients reveals distinct immunotypes with therapeutic implications. Science 369, eabc8511.
Schulte-Schrepping et al. (2020) Severe COVID-19 is marked by a dysregulated myeloid cell compartment. Cell 182, 1419-1440.
Granja et al. (2021) ArchR is a scalable software package for integrative single-cell chromatin accessibility analysis. Nat. Genet. 53, 403-411.
Feng et al. (2012) Identifying ChIP-seq enrichment using MACS. Nat. Protoc. 7, 1728-1740.
Li et al. (2021) Epigenetic landscapes of single-cell chromatin accessibility and transcriptomic immune profiles of T cells in COVID-19 patients. Front Immunol. 12, 625881 (2021).
Jung et al. (2019) A compendium of promoter-centered long-range chromatin interactions in the human genome. Nat. Genet. 51, 1442-1449.
Chen et al. (2021) Tissue-specific enhancer functional networks for associating distal regulatory regions to disease. Cell Syst. 12, 353-362.
Yao et al. (2021) Cell-type-specific immune dysregulation in severely ill COVID-19 patients. Cell Rep. 34, 108590.
Unterman et al. (2022) Single-cell multi-omics reveals dyssynchrony of the innate and adaptive immune system in progressive COVID-19. Nat. Commun. 13, 440.
Magill et al (2018). Changes in prevalence of health care-associated infections in U.S. hospitals. N. Engl. J. Med. 379, 1732-1744.
Tong et al. (2015) Staphylococcus aureus infections: epidemiology, pathophysiology, clinical manifestations, and management. Clin. Microbiol Rev. 28, 603-661.
Marquez-Ortiz et al. (2014) USA300-related methicillin-resistant Staphylococcus aureus clone is the predominant cause of community and hospital MRSA infections in Colombian children. Int J. Infect. Dis. 25, 88-93.
Hao et al. (2021) Integrated analysis of multimodal single-cell data. Cell 184, 3573-3587.
Skjeflo et al., (2014) Combined inhibition of complement and CD14 efficiently attenuated the inflammatory response induced by Staphylococcus aureus in a human whole blood model. J. Immunol. 192, 2857-2864.
Kusunoki et al. (1995) Molecules from Staphylococcus aureus that bind CD14 and stimulate innate immune responses. J. Exp. Med. 182, 1673-1682.
Ludwig, S. et al. (2001) Influenza virus-induced AP-1-dependent gene expression requires activation of the JNK signaling pathway. J. Biol. Chem. 276, 10990-10998.
Gjertsson et al. (2002) Impact of transcription factors AP-1 and NF-κB on the outcome of experimental Staphylococcus aureus arthritis and sepsis. Microbes Infect. 3, 527-534.
Liu. et al. (2011) Cistrome: an integrative platform for transcriptional regulation studies. Genome Biol. 12, R83.
Gillespie et al. (2022), The reactome pathway knowledgebase. Nucleic Acids Res. 50, D687-D692.
Kyriakis (1999), Activation of the AP-1 transcription factor by inflammatory cytokines of the TNF family. Gene Expr. 7, 217-231.
Hannemann et al. (2017), The AP-1 transcription factor c-Jun promotes arthritis by regulating cyclooxygenase-2 and arginase-1 expression in macrophages. J. Immunol. 198, 3605-3614.
Gasperini et al. (2019) A genome-wide framework for mapping gene regulation via cellular genetic screens. Cell 176, 377-390.
Consortium et al. (2020) Expanded encyclopaedias of DNA elements in the human and mouse genomes. Nature 583, 699-710.
Buniello et al. (2019) The NHGRI-EBI GWAS Catalog of published genome-wide association studies, targeted arrays and summary statistics 2019. Nucleic Acids Res. 47, D1005-D1012.
DeLorenze et al. (2016) Polymorphisms in HLA class II genes are associated with susceptibility to Staphylococcus aureus infection in a white population. J. Infect. Dis. 213, 816-823.
Chen et al. (2016) Genetic drivers of epigenetic and transcriptional variation in human immune cells. Cell 167, 1398-1414.
Ahn et al. (2013) Gene expression-based classifiers identify Staphylococcus aureus infection in mice and humans. PLoS One 8, e48979.
Ramilo et al (2007) Gene expression patterns in blood leukocytes discriminate patients with acute infections. Blood 109, 2066-2077.
Ardura et al. (2009) Enhanced monocyte response and decreased central memory T cells in children with invasive Staphylococcus aureus infections. PLoS One 4, e5446.
Cho et al. (2010) IL-17 is essential for host defense against cutaneous Staphylococcus aureus infection in mice. J. Clin. Invest. 120, 1762-1773.
Xiao et al. (2014) A novel significance score for gene selection and ranking. Bioinformatics 30, 801-807.
Chaussabel et al. (2008) A modular analysis framework for blood genomics studies: application to systemic lupus erythematosus. Immunity 29, 150-164.
Wenric and Shemirani (2018) Using supervised learning methods for gene selection in RNA-Seq case-control studies. Front. Genet. 9, 297.
Love et al. (2014) Moderated estimation of fold change and dispersion for RNA-seq data with DESeq2. Genome Biol. 15, 550.
Korsunsky et al. (2019) Fast, sensitive and accurate integration of single-cell data with Harmony. Nat. Methods 16, 1289-1296.
Squair et al. (201) Confronting false discoveries in single-cell differential expression. Nat. Commun. 12, 5692.
Schep et al. (2017) chromVAR: inferring transcription-factor-associated accessibility from single-cell epigenomic data. Nat. Methods 14, 975-978.
Anderson & Gusella (1984) Use of cyclosporin A in establishing Epstein-Barr virus-transformed human lymphoblastoid cell lines. Vitro 20, 856-858.
Tan et al. (2018), Three-dimensional genome structures of single diploid human cells. Science 361, 924-928.
McArthur and Capra (2021), Topologically associating domain boundaries that are stable across diverse cell types are evolutionarily constrained and enriched for heritability. Am. J. Hum. Genet 108, 269-283.
Rao et al. (2014), A 3D map of the human genome at kilobase resolution reveals principles of chromatin looping. Cell 159, 1665-1680.
Shin et al. (2016), TopDom: an efficient and deterministic method for identifying topological domains in genomes. Nucleic Acids Res. 44, e70.
Letizia et al. (2021), SARS-CoV-2 seropositivity and subsequent infection risk in healthy young adults: a prospective cohort study. Lancet Respir. Med. 9, 712-720.
Schmidt et al. (2015) GREGOR: evaluating global enrichment of trait-associated variants in epigenomic features using a systematic, data-driven approach. Bioinformatics 31, 2601-2606.
Chen (2023) Source data for paper “Mapping disease regulatory circuits at cell-type resolution from single-cell multiomics data”. Zenodo doi.org/10.5281/zenodo.7992711.
Chen, MAGICAL (v1.1). Zenodo doi.org/10.5281/zenodo.7951577 (2023).

### REFERENCES FOR PART 2

Balnis et al. (2021) Blood DNA methylation and COVID-19 outcomes. Clin Epigenetics 13: 118.
Bannister et al. (2022) Neonatal BCG vaccination is associated with a long-term DNA methylation signature in circulating monocytes. Sci Adv 8: eabn4002.
Behrens et al. (2020) The susceptibility to other infectious diseases following
measles during a three year observation period in Switzerland. Pediatr Infect Dis J 39: 478-482.
Castro de Moura et al. (2021) Epigenome-wide association study of COVID-19 severity with respiratory failure. EBioMedicine 66: 103339.
Chang et al. (2021) New-onset IgG autoantibodies in hospitalized patients with COVID-19. Nat Commun 12: 5417.
Chen et al. (2018) Longitudinal personal DNA methylome dynamics in a human with a chronic condition. Nat Med 24: 1930-1939.
Corley et al. (2021) Genome-wide DNA methylation profiling of peripheral blood reveals an epigenetic signature associated with severe COVID-19. J Leukoc Biol 110: 21-26.
DeDiego et al., (2019a) Novel functions of IFI44L as a feedback regulator of host antiviral responses. J Virol 93: e01159-19.
DeDiego et al. (2019b) Interferoninduced protein 44 interacts with cellular FK506-binding protein 5, negatively regulates host antiviral responses, and supports virus replication. mBio 10: e01839-19
Duttke et al. (2019) Identification and dynamic quantification of regulatory elements using total RNA. Genome Res 29: 1836-1846.
Friedman et al. (2010) Regularization Paths for Generalized Linear Models via Coordinate Descent. J Stat Softw 33: 1-22.
Furukawa et al. (2016) Intraindividual dynamics of transcriptome and genome-wide stability of DNA methylation. Sci Rep 6: 26424.
Heinz et al. (2010) Simple combinations of lineage-determining transcription factors prime cis-regulatory elements required for macrophage and B cell identities. Mol Cell 38: 576-589.
Horvath and Raj (2018) DNA methylation-based biomarkers and the epigenetic clock theory of ageing. Nat Rev Genet 19: 371-384.
Houseman et al. (2012) DNA methylation arrays as surrogate measures of cell mixture distribution. BMC Bioinformatics 13: 86.
Illumina (2014) Infinium MethylationEPIC Manifest Column Headings.
Johnson and Rabinovic (2007) Adjusting batch effects in microarray expression data using empirical Bayes methods. Biostatistics 8: 118-127
Konigsberg et al. (2021) Host methylation predicts SARS-CoV-2 infection and clinical outcome. Communications Medicine 1: 42.
Lee and Ashkar (2018) The dual nature of type I and type II interferons. Front Immunol. 9: 2061.
Leng and Muller (2006) Classification using functional data analysis for temporal gene expression data. Bioinformatics 22: 68-76.
Leodolter et al. (2021) IncDTW: An R package for incremental calculation of dynamic time warping. Journal of Statistical Software 99: 1-23.
Letizia et al. (2021) SARS-CoV-2 seropositivity and subsequent infection risk in healthy young adults: a prospective cohort study. Lancet Respir Med 9: 712-720.
Liberzon et al. (2015) The Molecular Signatures Database (MSigDB) hallmark gene set collection. Cell Syst 1: 417-425.
Liberzon et al. (2011) Molecular signatures database (MSigDB) 3.0. Bioinformatics 27: 1739-1740.
Liu and Muller(2003) Modes and clustering for time-warped gene expression profile data. Bioinformatics 19: 1937-1944.
Liu et al. (2020) Longitudinal characteristics of lymphocyte responses and cytokine profiles in the peripheral blood of SARS-CoV-2 infected patients. EBioMedicine 55: 102763.
Logue et al., (2021) Sequelae in Adults at 6 Months After COVID-19 Infection. JAMA Netw Open 4: e210830.
Lu et al. (2019) DNA methylation GrimAge strongly predicts lifespan and healthspan. Aging (Albany NY) 11: 303-327.
McNab et al. (2015) Type I interferons in infectious disease. Nat Rev. Immunol 15: 87-103.
Malkova et al., (2021) Post COVID-19 Syndrome in Patients with Asymptomatic/Mild Form. Pathogens 10.
Netea et al. (2020) Defining trained immunity and its role in health and disease. Nat Rev Immunol 20: 375-388.
Newman et al. (2015) Robust enumeration of cell subsets from tissue expression profiles. Nat Methods 12: 453-457.
Newman et al. (2019) Determining cell type abundance and expression from bulk tissues with digital cytometry. Nat. Biotechnol. 37: 773-782.
Pavli et al. (2021) Post-COVID Syndrome: Incidence, Clinical Spectrum, and Challenges for Primary Healthcare Professionals. Arch Med Res 52: 575-581.
Pohl and Beato (2014) bwtool: a tool for bigWig files. Bioinformatics 30: 1618-1619.
Ramos et al (2021) Antibody Responses to SARS-CoV-2 Following an Outbreak Among Marine Recruits With Asymptomatic or Mild Infection. Front Immunol 12: 681586.
Ritchie et al. (2015) limma powers differential expression analyses for RNA-sequencing and microarray studies. Nucleic Acids Res 43: e47.
Ronnblom and Leonoard (2019) Interferon pathway in SLE: one key to unlocking the mystery of the disease. Lupus Sci Med 6: e000270
Roy et al. (2021) DNA methylation signatures reveal that distinct combinations of transcription factors specify human immune cell epigenetic identity. Immunity 54: 2465-2480 e2465.
Sah et al. (2021) Asymptomatic SARS-CoV-2 infection: A systematic review and meta-analysis. Proc Natl Acad Sci USA 118.
Salas et al. (2022) Enhanced cell deconvolution of peripheral blood using DNA methylation for high-resolution immune profiling. Nature Com. 13: 763.
Simon et al, (2003) Pitfalls in the use of DNA microarray data for diagnostic and prognostic classification. J. Natl. Cancer Inst 95: 14-18.
Stuart et al., (2019) Comprehensive Integration of Single-Cell Data. Cell 177: 1888-1902 e1821.
Stunnenberg, International Human Epigenome C, Hirst M (2016) The International Human Epigenome Consortium: A Blueprint for Scientific Collaboration and Discovery. Cell 167: 1145-1149.
Su et al., (2022) Multiple Early Factors Anticipate Post-Acute COVID-19 Sequelae. Cell 185(5): 881-895.
Teschendorff (2019) Avoiding common pitfalls in machine learning omic data science. Nat. Mater. 18:422-427.
Thompson et al. (2022) Methylation risk scores are associated with a collection of phenotypes within electronic health record systems. medRxiv: 2022.2002.2007.22270047.
Tian et al. (2017) ChAMP: updated methylation analysis pipeline for Illumina BeadChips. Bioinformatics 33: 3982-3984.
Yousefi et al. (2022) DNA methylation-based predictors of health: applications and statistical considerations. Nat Rev Genet.
Zhou et al. (2021) An epigenome-wide DNA methylation study of patients with COVID-19. Ann Hum Genet 85: 221-234.

### REFERENCES FOR PART 3

Ferrer et al. (2014) Empiric anti-biotic treatment reduces mortality in severe sepsis and septic shock from the first hour: results from a guideline-based performance improvement program. Crit. Care Med. 42, 1749-1755.
CDC (2020). Antibiotic Resistance is a National Priority (Centers for Disease Control and Prevention). https://www.cdc.gov/drugresistance/us-activities.html.
Killingley et al. (2022). Safety, tolerability and viral kinetics during SARS-CoV-2 human challenge in young adults. Nat. Med. 28, 1031-1041.
Kucirka et al. (2020). Variation in false-negative rate of reverse transcriptase polymerase chain reaction-based SARS-CoV-2 tests by time since exposure. Ann. Intern. Med. 173, 262-267.
Self et al. (2017). Procalcitonin as a marker of etiology in adults hospitalized with community-acquired pneumonia. Clin. Infect. Dis. 65, 183-190.
Ramilo et al. (2007). Gene expression patterns in blood leukocytes discriminate pa-tients with acute infections. Blood 109, 2066-2077.
Suarez et al. (2015). Superiority of transcriptional profiling over procalcitonin for distinguishing bacterial from viral lower respiratory tract infections in hospitalized adults. J. Infect. Dis. 212, 213-222.
Sweeney et al. (2016). Robust classification of bacterial and viral infections via integrated host gene expression diagnostics. Sci. Transl. Med. 8, 346ra91.
Tsalik et al. (2021). Discriminating bacterial and viral infection using a rapid Host Gene Expression Test. Crit. Care Med. 49, 1651-1663.
Warsinske et al. (2019). Host-response-based gene signatures for tuberculosis diagnosis: a systematic comparison of 16 signatures. PLoS Med. 16, e1002786.
Tato and Khatri (2015). Integrated, multi-cohort analysis iden-tifies conserved transcriptional signatures across multiple respiratory vi-ruses. Immunity 43, 1199-1211.
Davenport et al. (2015). Transcriptomic profiling facilitates classification of response to influenza challenge. J. Mol. Med. (Berl.) 93, 105-114.
Parnell et al. (2012). A distinct influenza infection signature in the blood transcriptome of patients with severe community-acquired pneumonia. Crit. Care 16, R157.
Tang et al. (2017). A novel immune biomarker IFI27 discriminates between influenza and bacteria in patients with suspected respiratory infection. Eur. Respir. J. 49, 1602098.
Zaas et al. (2009). Gene expression signatures diagnose influenza and other symptomatic respiratory viral infections in humans. Cell Host Microbe 6, 207-217. https://doi.org/10.1016/j.chom.2009.07.006.
Huang et al. (2011). Temporal dynamics of host molecular responses differentiate symptom-atic and asymptomatic influenza A infection. PLOS Genet. 7.
McNab et al. (2015). Type I interferons in infectious disease. Nat. Rev. Immunol. 15, 87-103.
Bodkin et al., (2022). Systematic comparison of published host gene expression signatures for bacterial/viral discrimination. Genome Med. 14, 18.
Tsalik et al. (2016). Host gene expression classifiers diagnose acute respiratory illness etiology. Sci. Transl. Med. 8, 322ra11.
Herberg et al. (2016). Diagnostic test accuracy of a 2-transcript Host RNA Signature for Discriminating Bacterial vs Viral Infection in Febrile Children. JAMA 316, 835-845.
Smith et al. (2012). Identification of common biological pathways and drug targets across multiple respiratory viruses based on human host gene expression analysis. PLoS One 7, e33174.
Smith et al., (2013). Host response to respiratory bacterial pathogens as identified by integrated analysis of human gene expression data. PLoS One 8, e75607.
Statnikov et al. (2010). Improving development of the molecular signature for diagnosis of acute respiratory viral infections. Cell Host Microbe 7, 100-101.
Hu et al. (2013). Gene expression profiles in febrile children with defined viral and bacterial infection. Proc. Natl. Acad. Sci. USA 110, 12792-12797.
Bhattacharya et al. (2017). Transcriptomic biomarkers to discriminate bacterial from nonbacterial infection in adults hospitalized with respiratory illness. Sci. Rep. 7, 6548.
Zhu et al. (2014). Antiviral activity of human OASL protein is mediated by enhancing signaling of the RIG-I RNA sensor. Immunity 40, 936-948.
Barrett et al. (2013). NCBI GEO: archive for functional genomics data sets-update. Nucleic Acids Res. 41, D991-D995.
Frasca and Blomberg (2017). Adipose tissue inflammation in-duces B cell inflammation and decreases B cell function in aging. Front. Immunol. 8, 1003.
29. Pereira, B. I., and Akbar, A. N. (2016). Convergence of innate and adaptive immunity during human aging. Front. Immunol. 7, 445. https://doi.org/10. 3389/fimmu.2016.00445.
Kauffmann et al. (2009). arrayQualityMetrics—a bioconductor package for quality assessment of microarray data. Bioinformatics 25, 415-416.
Haynes et al. (2017) Empowering multi-cohort gene expression analysis to increase reproducibility. Pac. Symp. Biocomput. 22, 144-153.
Sweeney et al. (2015). A comprehensive time-course-based multicohort analysis of sepsis and sterile inflammation reveals a robust diagnostic gene set. Sci. Transl. Med. 7, 287.
Sampson et al. (2017). A four-biomarker blood signature discriminates systemic inflammation due to viral infection versus other etiologies. Sci. Rep. 7, 2914.
Liu et al. (2016). An individualized predictor of health and disease using paired reference and target samples. BMC Bioinformatics 17, 47.
Iuliano et al. (2018). Estimates of global seasonal influenza-associated respiratory mor-tality: a modelling study. Lancet 391, 1285-1300.
Emmerich and Deutz (2018). A tutorial on multiobjective optimization: fundamentals and evolutionary methods. Nat. Comput. 17, 585-609.
Berry et al. (2010). An interferon-inducible neutrophil-driven blood transcriptional signature in human tuberculosis. Nature 466, 973-977.
Holcomb et al. (2017). Host-Based Peripheral Blood Gene Expression Analysis for Diagnosis of Infectious Diseases. J. Clin. Microbiol. 55, 360-368.
Cappuccio et al. (2022). Multi-objective optimization identifies a specific and interpretable COVID-19 host response signature. Cell Syst.
Wickham et al. (2019). Welcome to the tidyverse. J. Open Source Software 4, 1686.
Ritchie et al. (2015). limma powers differential expression analyses for RNA-sequencing and microarray studies. Nucleic Acids Res. 43, e47.
Bolstad et al. (2003). A comparison of normalization methods for high density oligonucleotide array data based on variance and bias. Bioinformatics 19, 185-193.
Davis and Meltzer (2007). GEOquery: a bridge between the Gene Expression Omnibus (GEO) and BioConductor. Bioinformatics 23, 1846-1847.
Pages et al. (2020). AnnotationDbi: manipulation of SQLite-based annotations in bioconductor. https://aur. archlinux.org/r-annotationdbi.git.
Kuleshov et al. (2016). Enrichr: a comprehensive gene set enrichment analysis web server 2016 update. Nucleic Acids Res. 44, W90-W97.
Collado-Torres et al. (2017). Reproducible RNA-seq analysis using recount2. Nat. Biotechnol. 35, 319-321.
Mason (2002). Areas beneath the Relative Operating Characteristics (ROC) and Relative Operating Levels (ROL) Curves: Statistical Significance and Interpretation. Quarterly Journal of the Royal Meteorological Society 128 ((584):), 2145-2166.
Kuhn, (2008). Building Predictive Models in R Using the caret Package. J. Stat. Software 28, 1-26.

### REFERENCES FOR PART 4

Abbas et al. (2005). Immune response in silico (IRIS): immune-specific genes identified from a compendium of microarray expression data. Genes Immun. 6, 319-331.
Abbas et al. (2009). Deconvolution of blood microarray data identifies cellular activation patterns in systemic lupus erythematosus. PLoS ONE 4, e6098.
Andres-Terre et al., (2015). Integrated, Multi-cohort Analysis Identifies Conserved Transcriptional Signatures across Multiple Respiratory Viruses. Immunity 43, 1199-1211.
Arunachalam et al. (2020). Systems biological assessment of immunity to mild versus severe COVID-19 infection in humans. Science 369, 1210-1220.
Aschenbrenner et al. (2021). Disease severity-specific neutrophil signatures in blood transcriptomes stratify COVID-19 patients. Genome Med. 13, 7.
Azad et al. (2018). Inflammatory macrophage-associated 3-gene signature predicts subclinical allograft injury and graft survival. JCI Insight 3.
Bergamaschi et al. (2021). Longitudinal analysis reveals that delayed bystander CD8+ T cell activation and early immune pathology distinguish severe COVID-19 from mild disease. Immunity 54, 1257-1275.e8.
Bhaskaran et al. (2021). Factors associated with deaths due to COVID-19 versus other causes: population-based cohort analysis of UK primary care data and linked national death registrations within the OpenSAFELY platform. Lancet Reg. Health Eur. 6, 100109.
Bhattacharya et al. (2018). ImmPort, toward repurposing of open access immunological assay data for translational and clinical research. Sci. Data 5, 180015.
Blanco-Melo et al. (2020). Imbalanced Host Response to SARS-CoV-2 Drives Development of COVID-19. Cell 181, 1036-1045.e9
Bolen et al. (2011). Cell subset prediction for blood genomic studies. BMC Bioinformatics 12, 258.
Bongen et al. (2019). Sex Differences in the Blood Transcriptome Identify Robust Changes in Immune Cell Proportions with Aging and Influenza Infection. Cell Rep. 29, 1961-1973.e4.
Cappuccio et al. (2022). Earlier detection of SARS-CoV-2 infection by blood RNA signature microfluidics assay. Clin. and Transl. Disc. 2(3).
Chawla et al. (2022). Benchmarking transcriptional host response signatures for infection diagnosis. Cell Systems 13.
Chen et al. (2021). Tissue-specific enhancer functional networks for associating distal regulatory regions to disease. Cell Syst. 12, 353-362.e6.
COvid-19 Multi-omics Blood ATlas (COMBAT) Consortium. (2022). A blood atlas of COVID-19 defines hallmarks of disease severity and specificity. Cell 185, 916-938.e58.
Daamen et al. (2021). Comprehensive transcriptomic analysis of COVID-19 blood, lung, and airway. Sci. Rep. 11, 7052.
Dobin et al. (2013). STAR: ultrafast universal RNA-seq aligner. Bioinformatics 29, 15-21.
Emmerich and Deutz (2018). A tutorial on multiobjective optimization: fundamentals and evolutionary methods. Nat. Comput. 17, 585-609.
Fink (2012). Origin and Function of Circulating Plasmablasts during Acute Viral Infections. Front. Immunol. 3, 78.
Greene et al. (2015). Understanding multicellular function and disease with human tissue-specific networks. Nat. Genet. 47, 569-576.
Gupta et al. (2020). Extrapulmonary manifestations of COVID-19. Nat. Med. 26, 1017-1032.
Haynes et al. (2017). Empowering multi-cohort gene expression analysis to increase reproducibility. Pac. Symp. Biocomput. 22, 144-153.
Heinz et al. (2010). Simple combinations of lineage-determining transcription factors prime cis-regulatory elements required for macrophage and B cell identities. Mol. Cell 38, 576-589.
Holcomb et al. (2017). Host-Based Peripheral Blood Gene Expression Analysis for Diagnosis of Infectious Diseases. J. Clin. Microbiol. 55, 360-368.
Hong et al. (2019). Longitudinal profiling of human blood transcriptome in healthy and lupus pregnancy. J. Exp. Med. 216, 1154-1169.
Jassal et al. (2020). The Reactome Pathway Knowledgebase. Nucleic Acids Res. 48, D498-D503.
Langmead and Salzberg (2012). Fast gapped-read alignment with Bowtie 2. Nat. Methods 9, 357-359.
Law et al. (2014). voom: Precision weights unlock linear model analysis tools for RNA-seq read counts. Genome Biol. 15, R29.
Lee et al. (2020). Immunophenotyping of COVID-19 and influenza highlights the role of type I interferons in development of severe COVID-19. Sci. Immunol. 5.
Liao et al. (2014). featureCounts: an efficient general purpose program for assigning sequence reads to genomic features. Bioinformatics 30, 923-930.
de Lucena et al. (2020). Mechanism of inflammatory response in associated comorbidities in COVID-19. Diabetes Metab. Syndr. 14, 597-600.
Lydon et al. (2019a). Validation of a host response test to distinguish bacterial and viral respiratory infection. EBioMedicine 48, 453-461.
Lydon et al. (2019b). A host gene expression approach for identifying triggers of asthma exacerbations. PLoS ONE 14, e0214871.
McClain et al. (2021). Dysregulated transcriptional responses to SARS-CoV-2 in the periphery. Nat. Commun. 12, 1079.
Monaco et al. (2019). RNA-Seq Signatures Normalized by mRNA Abundance Allow Absolute Deconvolution of Human Immune Cell Types. Cell Rep. 26, 1627-1640.e7.
Moreira et al. (2021). Blood-based host biomarker diagnostics in active case finding for pulmonary tuberculosis: A diagnostic case-control study. EClinicalMedicine 33, 100776.
Nalbandian et al. (2021). Post-acute COVID-19 syndrome. Nat. Med. 27, 601-615.
Newman et al. (2015). Robust enumeration of cell subsets from tissue expression profiles. Nat. Methods 12, 453-457.
Ng et al. (2021). A diagnostic host response biosignature for COVID-19 from RNA profiling of nasal swabs and blood. Sci. Adv. 7.
Novershtern et al. (2011). Densely interconnected transcriptional circuits control cell states in human hematopoiesis. Cell 144, 296-309.
Phua et al. (2020). Intensive care management of coronavirus disease 2019 (COVID-19): challenges and recommendations. Lancet Respir. Med. 8, 506-517.
Rinchai et al. (2020). A modular framework for the development of targeted Covid-19 blood transcript profiling panels. J. Transl. Med. 18, 291.
Roy et al. (2018). A multi-cohort study of the immune factors associated with M. tuberculosis infection outcomes. Nature 560, 644-648.
Sampson et al. (2017). A Four-Biomarker Blood Signature Discriminates Systemic Inflammation Due to Viral Infection Versus Other Etiologies. Sci. Rep. 7, 2914.
Schulte-Schrepping et al. (2020). Severe COVID-19 Is Marked by a Dysregulated Myeloid Cell Compartment. Cell 182, 1419-1440.e23.
Schultheiβ et al. (2021). Maturation trajectories and transcriptional landscape of plasmablasts and autoreactive B cells in COVID-19. IScience 24, 103325.
Södersten et al. (2021). Diagnostic Accuracy Study of a Novel Blood-Based Assay for Identification of Tuberculosis in People Living with HIV. J. Clin. Microbiol. 59.
Stephenson et al. (2021). Single-cell multi-omics analysis of the immune response in COVID-19. Nat. Med.
Subramanian et al. (2005). Gene set enrichment analysis: a knowledge-based approach for interpreting genome-wide expression profiles. Proc Natl Acad Sci USA 102, 15545-15550.
Su et al. (2022). Multiple early factors anticipate post-acute COVID-19 sequelae. Cell 185, 881-895.e20.
Sweeney et al. (2016). Robust classification of bacterial and viral infections via integrated host gene expression diagnostics. Sci. Transl. Med. 8, 346ra91.
Sweeney et al. (2018). Validation of the sepsis metascore for diagnosis of neonatal sepsis. J. Pediatric Infect. Dis. Soc. 7, 129-135.
Tay et al. (2020). The trinity of COVID-19: immunity, inflammation and intervention. Nat. Rev. Immunol. 20, 363-374.
Thair et al. (2021a). Transcriptomic similarities and differences in host response between SARS-CoV-2 and other viral infections. IScience 24, 101947.
Thair et al. (2021b). Gene Expression-Based Diagnosis of Infections in Critically Ill Patients-Prospective Validation of the SepsisMetaScore in a Longitudinal Severe Trauma Cohort. Crit. Care Med. 49(8):e751-e760.
Tsalik et al. (2021). Discriminating bacterial and viral infection using a rapid host gene expression test. Crit. Care Med. 49, 1651-1663.
Turner et al. (2021). SARS-CoV-2 infection induces long-lived bone marrow plasma cells in humans. Nature 595, 421-425.
Warsinske et al. (2019). Host-response-based gene signatures for tuberculosis diagnosis: A systematic comparison of 16 signatures. PLoS Med. 16, e1002786.
Williamson et al. (2020). Factors associated with COVID-19-related death using OpenSAFELY. Nature 584, 430-436.
Xiong et al. (2020). Transcriptomic characteristics of bronchoalveolar lavage fluid and peripheral blood mononuclear cells in COVID-19 patients. Emerg. Microbes Infect. 9, 761-770.
Zhang et al. (2008). Model-based analysis of ChIP-Seq (MACS). Genome Biol. 9, R137.
Zheng et al. (2021). Multi-cohort analysis of host immune response identifies conserved protective and detrimental modules associated with severity across viruses. Immunity 54, 753-768.e5.

### REFERENCES FOR PART 5

Agius et al. (2020) Machine learning can identify newly diagnosed patients with CLL at high risk of infection. Nat. Commun., 11, 363.
Avey et al. (2017) Multiple network-constrained regressions expand insights into influenza vaccination responses. Bioinformatics, 33, i208-i216.
Camacho et al. (2018) Next-Generation Machine Learning for Biological Networks. Cell, 173, 1581-1592.
Fourati et al. (2018) A crowdsourced analysis to identify ab initio molecular signatures predictive of susceptibility to viral infection. Nat. Commun., 9, 4418.
Gold, et al. (2019) Shallow Sparsely-Connected Autoencoders for Gene Set Projection. Pac. Symp. Biocomput., 24, 374-385.
Kang et al. (2017) A biological network-based regularized artificial neural network model for robust phenotype prediction from gene expression data. BMC Bioinformatics, 18, 565.
Kaforou et al. (2013) Detection of tuberculosis in HIV-infected and-uninfected African adults using whole blood RNA expression signatures: a case-control study. PLoS medicine 10.10 (2013).
Kuleshov et al. (2016) Enrichr: a comprehensive gene set enrichment analysis web server 2016 update. Nucleic Acids Res., 44, W90-7.
Lin, et al. (2017) Using neural networks for reducing the dimensions of single-cell RNA-Seq data. Nucleic Acids Res., 45, e156.
Mao et al. (2019) Pathway-level information extractor (PLIER) for gene expression data. Nat. Methods, 16, 607-610.
Murdoch et al. (2019) Definitions, methods, and applications in interpretable machine learning. Proc Natl Acad Sci USA, 116, 22071-22080.
Patel-Murray et al. (2020) A Multi-Omics Interpretable Machine Learning Model Reveals Modes of Action of Small Molecules. Sci. Rep., 10, 954.
Peng et al. (2019) Combining gene ontology with deep neural networks to enhance the clustering of single cell RNA-Seq data. BMC Bioinformatics, 20, 284.
Stoney et al. (2018) Using set theory to reduce redundancy in pathway sets. BMC Bioinformatics, 19, 386.
Subramanian et al. (2005) Gene set enrichment analysis: a knowledge-based approach for interpreting genome-wide expression profiles. Proc Natl Acad Sci USA, 102, 15545-15550.
Taroni, et al. (2019) Multiplier: A transfer learning framework for transcriptomics reveals systemic features of rare disease. Cell Syst., 8, 380-394.e4.
Zhang et al. (2022) Single nucleus transcriptome and chromatin accessibility of postmortem human pituitaries reveal diverse stem cell regulatory mechanisms. Cell Rep. 8;38(10):110467.

### REFERENCES FOR PART 6

Kim et al. (2009) Transcriptional Regulatory Circuits: Predicting Numbers from Alphabets. Science 325, 429-432.
Ma et al. (2020) Chromatin Potential Identified by Shared Single-Cell Profiling of RNA and Chromatin. Cell 183, 1103-1116.e20.
Chen et al. (2019) High-throughput sequencing of the transcriptome and chromatin accessibility in the same cell. Nat. Biotechnol. 37, 1452-1457.
Stuart et al. (2021) Single-cell chromatin state analysis with Signac. Nat. Methods 18, 1333-1341.
Schep et al. (2017) ChromVAR: Inferring transcription-factor-associated accessibility from single-cell epigenomic data. Nat. Methods 14, 975-978.
Bravo González-Blas et al. (2019) cisTopic: cis-regulatory topic modeling on single-cell ATAC-seq data. Nat. Methods 16, 397-400.
Nakato and Shirahige (2016) Recent advances in ChIP-seq analysis: from quality management to whole-genome annotation. Brief Bioinform. bbw023.
Landt et al. (2012) ChIP-seq guidelines and practices of the ENCODE and modENCODE consortia. Genome Res. 22, 1813-1831.
Schmidt et al. (2010) A CTCF-independent role for cohesin in tissue-specific transcription. Genome Res. 20, 578-588.
Wen et al. (2017) Integrating molecular QTL data into genome-wide genetic association analysis: Probabilistic assessment of enrichment and colocalization. PLOS Genet. 13, e1006646.
Gao and Qian (2019) EnhancerAtlas 2.0: an updated resource with enhancer annotation in 586 tissue/cell types across nine species, Nucleic Acids Research.
Jiang et al. (2022) Nonparametric single-cell multiomic characterization of trio relationships between transcription factors, target genes, and cis-regulatory regions. Cell Syst. 13, 737-751.e4.
Hao et al. (2021) Integrated analysis of multimodal single-cell data. Cell 184, 3573-3587.e29.
Wu et al. (2014) NAR Breakthrough Article: Three-tiered role of the pioneer factor GATA2 in promoting androgen-dependent gene expression in prostate cancer. Nucleic Acids Res. 42, 3607.
Schang et al. (2022) Transcription factor GATA2 may potentiate follicle-stimulating hormone production in mice via induction of the BMP antagonist gremlin in gonadotrope cells. J. Biol. Chem. 298.
Folon et al. (2023) Contribution of heterozygous PCSK1 variants to obesity and implications for precision medicine: a case-control study. Lancet Diabetes Endocrinol. 11, 182-190.
Frank et al. (2013) Severe obesity and diabetes insipidus in a patient with PCSK1 deficiency, Molecular Genetics and Metabolism 110(1-2), pp. 191-194.
Wei et al. (2014) Genetic Variants in PCSK1 Gene Are Associated with the Risk of Coronary Artery Disease in Type 2 Diabetes in a Chinese Han Population: A Case Control Study, PLoS One 9(1): e87168.
Ware et al. (1992) Expression of surface lymphotoxin and tumor necrosis factor on activated T, B, and natural killer cells. J. Immunol. Baltim. Md 1950 149, 3881-3888.
Ohshima et al. (1999), Naive human CD4+ T cells are a major source of lymphotoxin alpha. J. Immunol. Baltim. Md 1950 162, 3790-3794.
Ruf-Zamojski, et al. (2021) Single nucleus multi-omics regulatory landscape of the murine pituitary. Nat. Commun. 12, 2677.
Mendelev et al. (2022) Multi-omics profiling of single nuclei from frozen archived postmortem human pituitary tissue. STAR Protoc. 3, 101446.

### REFERENCES FOR PART 7

Mao et al. (2019) Pathway-level information extractor (PLIER) for gene expression data. Nat Methods. 2019 July; 16(7):607-610.

### CONCLUSION

The terminology used herein is for the purpose of describing particular cases and is not intended to be limiting. As used herein, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated. The implementations were chosen and described in order to best explain the principles and their practical applications, to thereby enable others skilled in the art to best utilize the implementations and various implementations with various modifications as are suited to the particular use contemplated.

What is claimed:

1. A method for constructing a model that determines whether a subject is afflicted with a condition, the method comprising:
A) for each respective first subject in a first plurality of subjects not afflicted with the condition,
obtaining a first RNA-seq dataset comprising a respective discrete attribute value for each gene transcript in a corresponding first plurality of gene transcripts, for each cell in a respective first plurality of cells from a corresponding first biological sample from the respective first subject, and
obtaining a first ATAC-seq dataset comprising a respective ATAC fragment count for each corresponding ATAC peak in a corresponding first plurality of ATAC peaks, for each respective cell in a respective second plurality of cells from a corresponding second biological sample from the respective subject;
B) for each respective second subject in a second plurality of subjects afflicted with the condition,
obtaining a second RNA-seq dataset comprising a respective discrete attribute value for each gene transcript in a corresponding second plurality of gene transcripts, for each cell in a respective third plurality of cells from a corresponding third biological sample from the respective second subject, and
obtaining a second ATAC-seq dataset comprising a respective ATAC fragment count for each ATAC peak in a corresponding second plurality of ATAC peaks, for each respective cell in a respective fourth plurality of cells from a corresponding fourth biological sample from the respective subject;
C) using the first RNA-seq dataset and the second RNA-seq dataset to identify a plurality of candidate genes having differential transcription;
D) using the first ATAC-seq dataset and the second ATAC-seq dataset to identify a plurality of candidate ATAC peaks having differential accessibility between the first plurality of subjects and the second plurality of subjects;
E) for each respective transcription factor motif in a plurality of transcription factor motifs, mapping the respective transcription factor motif onto the plurality of candidate ATAC peaks form a plurality of mapped transcription factor motifs; and
F) constructing the model that determines whether a subject is afflicted with a condition using ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

2. The method of claim 1, wherein each respective first plurality of cells comprises 50 cells, each respective second plurality of cells comprises 50 cells, each respective third plurality of cells comprises 50 cells, and each respective fourth plurality of cells comprises 50 cells.

3. The method of claim 1 or 2, wherein each corresponding first plurality of gene transcripts represents 50 or more genes, each corresponding first plurality of ATAC peaks comprises 50 or more peaks, each corresponding second plurality of gene transcripts represents 50 or more genes, each corresponding second plurality of ATAC peaks comprises 50 or more peaks.

4. The method of any one of claims 1-3, wherein the plurality of candidate genes having differential transcription comprises 50 or more candidate genes, and the plurality of candidate ATAC peaks having differential accessibility comprises 50 or more candidate peaks.

5. The method of any one of claims 1-4, wherein the first plurality of subjects comprises 25 or more subjects and the second plurality of subjects comprises 25 or more subjects.

6. The method of any one of claims 1-5, wherein the first RNA-seq dataset is a single cell RNA-seq dataset, the second RNA-seq dataset is a single cell RNA-seq dataset, the first ATAC-seq dataset is a single cell ATAC-seq dataset, and the second ATAC-seq dataset is a single cell ATAC-seq dataset.

7. The method of any one of claims 1-5, wherein the first RNA-seq dataset is a bulk RNA-seq dataset, the second RNA-seq dataset is a bulk RNA-seq dataset, the first ATAC-seq dataset is a bulk ATAC-seq dataset, and the second ATAC-seq dataset is a bulk ATAC-seq dataset.

8. The method of any one of claims 1-5, wherein the first RNA-seq dataset, the second RNA-seq dataset, the first ATAC-seq dataset, and the second ATAC-seq dataset are determined using cells from the first and second plurality of subjects that have a common cell type.

9. The method of claim 8, wherein the common cell type is B memory, B naïve, CD4 TCM, CD8 Naïve, CD8 TEM, CD14 Mono, CD16 Mono, cDC2, MAIT, NK, NK_CD56bright, Platelets, CD14 monocytes, CD16 monocytes, CD4 TCM cells, CD8 TEM cells, CD4 Naïve cells, or natural killer.

10. The method of any one of claims 1-9, wherein a candidate gene in the plurality of candidate genes satisfied the proximity threshold with respect to a respective candidate ATAC peak when the candidate gene is within 20 kilobases, within 15 kilobases, within 10 kilobases, or within 5 kilobases of the respective candidate ATAC peak in a reference genome for the first and second plurality of subjects.

11. The method of claim 10, wherein the reference genome is a human reference genome.

12. The method of any one of claims 1-11, wherein the condition is a pathogenic infection.

13. The method of claim 12, wherein the pathogenic infection is a Covid infection or a Staph infection.

14. The method of claim 12, wherein the pathogenic infection is a bacterial infection or a viral infection.

15. The method of any one of claims 1-15, wherein the condition is a disease.

16. The method of any one of claims 1-15, wherein the forming F) uses Bayesian analysis of ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

17. The method of any one of claims 1-16, wherein the model comprises 1000, 10,000, 100,000 or 1×10⁶ parameters.

18. The method of any one of claims 1-17, wherein
only data for a first cell type is used by the using C) to identify a plurality of candidate genes having differential transcriptions and
only data for the first cell type is used by the using D) to identify the plurality of candidate ATAC peaks having differential accessibility, wherein optionally the first cell type is CD8 effector memory T cells, CD14 monocytes, or natural killer cells.

19. A computer system for constructing a model that determines whether a subject is afflicted with a condition, the computer system comprising:
one or more processors; and
memory addressable by the one or more processors, the memory storing at least one program for execution by the one or more processors, the at least one program comprising instructions for:
A) for each respective first subject in a first plurality of subjects not afflicted with the condition,
obtaining a first RNA-seq dataset comprising a respective discrete attribute value for each gene transcript in a corresponding first plurality of gene transcripts, for each cell in a respective first plurality of cells from a corresponding first biological sample from the respective first subject, and
obtaining a first ATAC-seq dataset comprising a respective ATAC fragment count for each corresponding ATAC peak in a corresponding first plurality of ATAC peaks, for each respective cell in a respective second plurality of cells from a corresponding second biological sample from the respective subject;
B) for each respective second subject in a second plurality of subjects afflicted with the condition,
obtaining a second RNA-seq dataset comprising a respective discrete attribute value for each gene transcript in a corresponding second plurality of gene transcripts, for each cell in a respective third plurality of cells from a corresponding third biological sample from the respective second subject, and
obtaining a second ATAC-seq dataset comprising a respective ATAC fragment count for each ATAC peak in a corresponding second plurality of ATAC peaks, for each respective cell in a respective fourth plurality of cells from a corresponding fourth biological sample from the respective subject;
C) using the first RNA-seq dataset and the second RNA-seq dataset to identify a plurality of candidate genes having differential transcription;
D) using the first ATAC-seq dataset and the second ATAC-seq dataset to identify a plurality of candidate ATAC peaks having differential accessibility between the first plurality of subjects and the second plurality of subjects;
E) for each respective transcription factor motif in a plurality of transcription factor motifs, mapping the respective transcription factor motif onto the plurality of candidate ATAC peaks form a plurality of mapped transcription factor motifs; and
F) constructing the model that determines whether a subject is afflicted with a condition using ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

20. A non-transitory computer readable storage medium, wherein the non-transitory computer readable storage medium stores instructions, which when executed by a computer system, cause the computer system to perform a method for constructing a model that determines whether a subject is afflicted with a condition, the method comprising:
A) for each respective first subject in a first plurality of subjects not afflicted with the condition,
obtaining a first RNA-seq dataset comprising a respective discrete attribute value for each gene transcript in a corresponding first plurality of gene transcripts, for each cell in a respective first plurality of cells from a corresponding first biological sample from the respective first subject, and
obtaining a first ATAC-seq dataset comprising a respective ATAC fragment count for each corresponding ATAC peak in a corresponding first plurality of ATAC peaks, for each respective cell in a respective second plurality of cells from a corresponding second biological sample from the respective subject;
B) for each respective second subject in a second plurality of subjects afflicted with the condition,
obtaining a second RNA-seq dataset comprising a respective discrete attribute value for each gene transcript in a corresponding second plurality of gene transcripts, for each cell in a respective third plurality of cells from a corresponding third biological sample from the respective second subject, and
obtaining a second ATAC-seq dataset comprising a respective ATAC fragment count for each ATAC peak in a corresponding second plurality of ATAC peaks, for each respective cell in a respective fourth plurality of cells from a corresponding fourth biological sample from the respective subject;
C) using the first RNA-seq dataset and the second RNA-seq dataset to identify a plurality of candidate genes having differential transcription;
D) using the first ATAC-seq dataset and the second ATAC-seq dataset to identify a plurality of candidate ATAC peaks having differential accessibility between the first plurality of subjects and the second plurality of subjects;
E) for each respective transcription factor motif in a plurality of transcription factor motifs, mapping the respective transcription factor motif onto the plurality of candidate ATAC peaks form a plurality of mapped transcription factor motifs; and
F) constructing the model that determines whether a subject is afflicted with a condition using ATAC-seq abundance data in the first and second RNA-seq dataset for those candidate genes in the plurality of candidate genes satisfying a proximity threshold with respect to a respective candidate ATAC peak to which a transcription factor motif in the plurality of transcription factor motifs mapped.

21. A method for determining whether a subject is afflicted with an S. aureses infection, the method comprising:
obtaining a plurality of discrete attribute values, wherein each discrete attribute value in the plurality of discrete attribute values represents a transcript abundance of a respective gene in a plurality of genes in a biological sample from the subject, wherein the plurality of genes comprises three or more genes listed in Table 1.13; and
inputting the plurality of discrete attribute values into a model comprising a plurality of parameters, wherein the model applies the plurality of parameters to the plurality of discrete attribute values to generate as output from the model an indication as to whether the subject is afflicted with the S. aureses infection.

22. The method of claim 21, wherein the plurality of discrete attribute values is obtained by bulk transcriptome sequencing of nucleic acids in the biological sample.

23. The method of claim 21, wherein a first gene in the plurality of genes is associated with the cell type CD14 Mono in Table 1.13.

24. The method of any one of claims 21-23, wherein a second gene in the plurality of genes is associated with the cell type CD16 Mono in Table 1.13.

25. The method of any one of claims 21-24, the method further comprising:
obtaining, in electronic form, a plurality of sequence reads from the biological sample, wherein the plurality of sequence reads comprises at least 10,000 RNA sequence reads; and
using the plurality of sequence reads to determine each discrete attribute value in the plurality of discrete attribute values.

26. The method of claim 26, wherein the using maps each respective sequence read in the plurality of sequence reads to a reference genome.

27. The method of any one of claims 21-26, wherein the biological sample is blood, whole blood, or plasma.

28. The method of any one of claims 25-27, wherein the biological sample comprises a plurality of mRNA molecules and the obtaining the plurality of sequence reads further comprises sequencing the plurality of mRNA molecules using RNA sequencing.

29. The method of any one of claims 21-28, wherein the plurality of sequence reads comprises at least 100,000, at least 1×10⁶, or at least 1×10⁷ sequence reads.

30. The method of any one of claims 21-29, wherein the model is selected from the group consisting of: a logistic regression model, a neural network, a support vector machine, a Naive Bayes model, a nearest neighbor model, a boosted trees model, a random forest model, a decision tree, or a clustering model.

31. The method of any one of claims 21-30, wherein the plurality of parameters comprises 100 or more parameters, 1000 or more parameters, 10,000 or more parameters, 100,000 or more parameters, or 1×10⁶ or more parameters.

32. The method of any one of claims 21-31, wherein the indication as to whether the subject is afflicted with the S. aureses infection is a likelihood that the subject is afflicted with the S. aureses infection.

33. The method of any one of claims 21-32, wherein the indication as to whether the subject is afflicted with the S. aureses infection is a binary indication as to whether or not the subject is afflicted with the S. aureses infection.

34. The method of any one of claims 21-26, or 28-33, wherein the biological sample comprises serum, urine, cerebrospinal fluid, fecal, saliva, sweat, tears, pleural fluid, pericardial fluid, or peritoneal fluid from the subject.

35. The method of any one of claims 21-26, or 28-33, wherein the biological sample consists of blood, whole blood, plasma, serum, urine, cerebrospinal fluid, fecal, saliva, sweat, tears, pleural fluid, pericardial fluid, or peritoneal fluid from the subject.

36. The method of any one of claims 1-35, the method further comprises treating the subject with a drug when the model indicates that the subject has an S. aureses infection.

37. The method of claim 36, wherein the drug is cefazolin, nafcillin, oxacillin, vancomycin, daptomycin, linezolid, or a combination thereof.

38. A method for determining whether a subject is afflicted with an antibiotic resistant S. aureses infection or an antibiotic sensitive S. aureses infection, the method comprising:
obtaining a plurality of discrete attribute values, wherein each discrete attribute value in the plurality of discrete attribute values represents a transcript abundance of a respective gene in a plurality of genes in a biological sample from the subject, wherein the plurality of genes comprises three or more genes listed in Table 1.14; and
inputting the plurality of discrete attribute values into a model comprising a plurality of parameters, wherein the model applies the plurality of parameters to the plurality of discrete attribute values to generate as output from the model an indication as to whether the subject is afflicted with an antibiotic resistant S. aureses infection or an antibiotic sensitive S. aureses infection.

39. The method of claim 38, wherein the plurality of discrete attribute values is obtained by bulk transcriptome sequencing of nucleic acids in the biological sample.

40. The method of 38, the method further comprising:
obtaining, in electronic form, a plurality of sequence reads from the biological sample, wherein the plurality of sequence reads comprises at least 10,000 RNA sequence reads; and
using the plurality of sequence reads to determine each discrete attribute value in the plurality of discrete attribute values.

41. The method of claim 40, wherein the using maps each respective sequence read in the plurality of sequence reads to a reference genome.

42. The method of any one of claims 38-41, wherein the biological sample is blood, whole blood, or plasma.

43. The method of any one of claims 38-41, wherein the biological sample comprises a plurality of mRNA molecules and the obtaining the plurality of sequence reads further comprises sequencing the plurality of mRNA molecules using RNA sequencing.

44. The method of any one of claims 40-43, wherein the plurality of sequence reads comprises at least 100,000, at least 1×10⁶, or at least 1×10⁷ sequence reads.

45. The method of any one of claims 38-44, wherein the model is selected from the group consisting of: a logistic regression model, a neural network, a support vector machine, a Naive Bayes model, a nearest neighbor model, a boosted trees model, a random forest model, a decision tree, or a clustering model.

46. The method of any one of claims 38-45, wherein the plurality of parameters comprises 100 or more parameters, 1000 or more parameters, 10,000 or more parameters, 100,000 or more parameters, or 1×10⁶ or more parameters.

47. The method of any one of claims 38-41, or 43-46, wherein the biological sample comprises serum, urine, cerebrospinal fluid, fecal, saliva, sweat, tears, pleural fluid, pericardial fluid, or peritoneal fluid from the subject.

48. The method of any one of claims 38-41, or 43-46, wherein the biological sample consists of blood, whole blood, plasma, serum, urine, cerebrospinal fluid, fecal, saliva, sweat, tears, pleural fluid, pericardial fluid, or peritoneal fluid from the subject.

49. The method of any one of claims 38-48, the method further comprises treating the subject with a drug when the model indicates that the subject is afflicted with an antibiotic sensitive S. aureses infection

50. The method of claim 49, wherein the drug is cefazolin, nafcillin, oxacillin, vancomycin, daptomycin, linezolid, or a combination thereof.

51. A method for determining whether a subject is afflicted with COVID-19, the method comprising:
obtaining a plurality of discrete attribute values, wherein each discrete attribute value in the plurality of discrete attribute values represents a transcript abundance of a respective gene in a plurality of genes in a biological sample from the subject, wherein the plurality of genes comprises three or more genes listed in FIG. 60; and
inputting the plurality of discrete attribute values into a model comprising a plurality of parameters, wherein the model applies the plurality of parameters to the plurality of discrete attribute values to generate as output from the model an indication as to whether the subject is afflicted with COVID-19.

52. The method of claim 51, wherein the plurality of discrete attribute values is obtained by bulk transcriptome sequencing of nucleic acids in the biological sample.

53. The method of claims 51 or 42, the method further comprising:
obtaining, in electronic form, a plurality of sequence reads from the biological sample, wherein the plurality of sequence reads comprises at least 10,000 RNA sequence reads; and
using the plurality of sequence reads to determine each discrete attribute value in the plurality of discrete attribute values.

54. The method of claim 53, wherein the using maps each respective sequence read in the plurality of sequence reads to a reference genome.

55. The method of any one of claims 51-54, wherein the biological sample is blood, whole blood, or plasma.

56. The method of any one of claims 51-55, wherein the biological sample comprises a plurality of mRNA molecules and the obtaining the plurality of sequence reads further comprises sequencing the plurality of mRNA molecules using RNA sequencing.

57. The method of any one of claims 53-56, wherein the plurality of sequence reads comprises at least 100,000, at least 1×10⁶, or at least 1×10⁷ sequence reads.

58. The method of any one of claims 51-57, wherein the model is selected from the group consisting of: a logistic regression model, a neural network, a support vector machine, a Naive Bayes model, a nearest neighbor model, a boosted trees model, a random forest model, a decision tree, or a clustering model.

59. The method of any one of claims 51-58, wherein the plurality of parameters comprises 100 or more parameters, 1000 or more parameters, 10,000 or more parameters, 100,000 or more parameters, or 1×10⁶ or more parameters.

60. The method of any one of claims 51-54, or 56-59, wherein the biological sample comprises serum, urine, cerebrospinal fluid, fecal, saliva, sweat, tears, pleural fluid, pericardial fluid, or peritoneal fluid from the subject.

61. The method of any one of claims 51-54, or 56-60, wherein the biological sample consists of blood, whole blood, plasma, serum, urine, cerebrospinal fluid, fecal, saliva, sweat, tears, pleural fluid, pericardial fluid, or peritoneal fluid from the subject.

62. The method of any one of claims 51-61, the method further comprises treating the subject with a drug when the model indicates that the subject is afflicted with COVID-19

63. The method of claim 62, wherein the drug is Nirmatrelvir, Ritonavir, Remdesvir, Molnupiravir, or a combination thereof, or a combination thereof.

64. A method for predicting a future severity of an infection or inflammatory disease in a subject afflicted with the infection or inflammatory disease, the method comprising:
obtaining a plurality of methylation levels, wherein each respective methylation level in the plurality of methylation levels represents a corresponding methylation level at a CpG site at a corresponding genetic locus in a plurality of genetic loci in a biological sample obtained from the subject; and
inputting the plurality of methylation levels into a model comprising a plurality of parameters, wherein the model applies the plurality of parameters to the plurality of methylation levels to generate as output from the model an indication as to future severity of an infection or inflammatory disease in the subject.

65. A method for predicting susceptibility a subject has to an infection in a subject presently free of the infection, the method comprising:
obtaining a plurality of methylation levels, wherein each respective methylation level in the plurality of methylation levels represents a corresponding methylation level at a CpG site at a corresponding genetic locus in a plurality of genetic loci in a biological sample obtained from the subject; and
inputting the plurality of methylation levels into a model comprising a plurality of parameters, wherein the model applies the plurality of parameters to the plurality of methylation levels to generate as output from the model the susceptibility the subject has to incurring a severe form of the infection upon exposure to the invention.

66. A method for predicting how long a subject has had an infection, the method comprising:
obtaining a plurality of methylation levels, wherein each respective methylation level in the plurality of methylation levels represents a corresponding methylation level aa CpG site at a corresponding genetic locus in a plurality of genetic loci in a biological sample obtained from the subject; and
inputting the plurality of methylation levels into a model comprising a plurality of parameters, wherein the model applies the plurality of parameters to the plurality of methylation levels to generate as output from the model a period of time the subject has had the infection.

67. The method of any one of claims 64-66, wherein the infection is a chronic hepatitis C virus infection, chronic human immunodeficiency virus infection, or SARS-CoV-2.

68. The method of claim 64, wherein the inflammatory disease is systemic lupus erythematosus, multiple sclerosis, rheumatoid arthritis, or inflammatory bowel disease.

69. The method of any one of claims 64-68, wherein each genetic loci in the plurality of genetic loci corresponds to a CpG site in a human genome.

70. The method of claim 69, wherein at least five genetic loci in the plurality of genetic loci are in FIG. 20B.

71. The method of any one of claims 64-70, wherein the biological sample is blood, whole blood, or plasma.

72. The method of any one of claims 64-71, wherein the plurality of methylation levels is obtained from sequencing a plurality of sequence reads of nucleic acids in the biological sample.

73. The method of claim 72 wherein the plurality of sequence reads comprises at least 10,000, at least 100,000, at least 1×10⁶, or at least 1×10⁷ sequence reads.

74. The method of any one of claims 64-73, wherein the model is selected from the group consisting of: a logistic regression model, a neural network, a support vector machine, a Naive Bayes model, a nearest neighbor model, a boosted trees model, a random forest model, a decision tree, or a clustering model.

75. The method of any one of claims 64-74, wherein the plurality of parameters comprises 100 or more parameters, 1000 or more parameters, 10,000 or more parameters, 100,000 or more parameters, or 1×10⁶ or more parameters.

76. The method of any one of claims 64-70, or 71-75, wherein the biological sample comprises serum, urine, cerebrospinal fluid, fecal, saliva, sweat, tears, pleural fluid, pericardial fluid, or peritoneal fluid from the subject.

77. The method of any one of claims 64-70, or 71-75, wherein the biological sample consists of blood, whole blood, plasma, serum, urine, cerebrospinal fluid, fecal, saliva, sweat, tears, pleural fluid, pericardial fluid, or peritoneal fluid from the subject.

78. The method of any one of claims 64-66 or 71-77, wherein the infection is SARS-CoV-2 and the plurality of CpG sites comprises 5 or more, 10 or more, 20 or more, 30 or more, 40 or more, or 50 or more CpG sites listed in Tables 2.3 or 2.4.

79. The method of claim 78 wherein a first CpG site in the plurality of CpG sites is a CpG site that is indicated to be hypomethylated during First-Control, Mid-Control, EarlyPost-Control, or Late Post-Control in Tables 2.3 or 2.4.

80. The method of claim 78 or 79 wherein a second CpG site in the plurality of CpG sites is a CpG site that is indicated to be hypermethylated during First-Control, Mid-Control, EarlyPost-Control, or Late Post-Control in Tables 2.3 or 2.4.

81. The method of any one of claims 64-66 or 71-77, wherein the infection is SARS-CoV-2 and the plurality of CpG sites comprises 5 or more, 10 or more, 20 or more, 30 or more, 40 or more, or 50 or more CpG sites listed in Tables 2.5 or 2.6.

82. The method of claim 81 wherein a first CpG site in the plurality of CpG sites is a CpG site that is indicated to be hypomethylated during Asymptomatic.Control-Symptomatic.Control, First-Symptomatic.First, Asymptomatic.Mid-Symptomatic.Mid, Asymptomatic.EarlyPost-Symptomatic.EarlyPost, or Asymptomatic.LatePost-Symptomatic.LatePost, in Tables 2.5 or 2.6.

83. The method of claim 81 or 82 wherein a second CpG site in the plurality of CpG sites is a CpG site that is indicated to be hypermethylated during Asymptomatic.Control-Symptomatic.Control, First-Symptomatic.First, Asymptomatic.Mid-Symptomatic.Mid, Asymptomatic.EarlyPost-Symptomatic.EarlyPost, or Asymptomatic.LatePost-Symptomatic.LatePost, in Tables 2.5 or 2.6.

84. The method of any one of claims 64-66 or 71-77, wherein the infection is SARS-CoV-2 and the plurality of CpG sites comprises 1, 2, 3, 4, 5, 6, 7, 8, 9, 10, 11, 12, 13, 14, 15, 16, 17, 18, 19, 20 or more CpG sites listed in FIG. 20B.

85. The method of any one of claims 65-84, wherein each genetic locus in the plurality of genetic loci consists of a single CpG site in the plurality of CpG sites.

86. The method of any one of claims 65-84, wherein each genetic locus in the plurality of genetic loci is less than 1000 nucleotides, less than 500 nucleotides, or less than 300 nucleotides in length.

87. The method of any one of claims 65-84, wherein each genetic locus in the plurality of genetic loci is between 50 and 500 nucleotides in length.

88. A method of evaluating a gene signature associated with a target condition that can afflict a host species, wherein the gene signature comprises a first plurality of positive genes that are up-regulated when the test subject has the target condition and a second plurality of genes that are down-regulated when the test subject has the target condition, the method comprising:
A) obtaining an indication of each gene in the first plurality of positive genes;
B) obtaining an indication of each gene in the second plurality of negative genes;
C) obtaining a plurality of datasets, wherein
each dataset in the plurality of datasets includes transcriptional data for each respective subject in a corresponding plurality of subjects and an indication of whether the respective subject has or does not have a respective test condition in a plurality of test conditions,
the plurality of datasets includes at least one dataset for each test condition in the plurality of test conditions,
at least one test condition in the plurality of test conditions is the target condition,
D) for each respective dataset in a plurality of datasets, for each respective time point in a set of time points represented by the respective dataset:
for each respective subject in the respective dataset, determining a score for the respective subject at the respective time point by determining a difference between a geometric mean of abundance values for the first plurality of positive genes and a geometric mean of abundance values for the second plurality of positive genes indicated in the respective dataset,
determining an area under a receiver operator characteristic curve (AUROC) value for the respective dataset for the test condition using the respective score for each subject in the respective dataset at each respective timepoint;
E) evaluating a performance of the gene signature using the AUROC value of each dataset in the plurality of datasets associated with the target condition; and
F) evaluating a cross-reactivity of the gene signature from the AUROC value of each dataset in the plurality of datasets associated with a test condition that is other than the target condition.

89. The method of claim 88, wherein the plurality of datasets comprises 10 or more datasets, 100 or more datasets, 1000 or more datasets, or 10,000 or more datasets.

90. The method of claim 88 or 89, wherein the target condition an infection from a predetermined virus species.

91. The method of claim 88 or 89, wherein the target condition an infection from a predetermined bacterial species.

92. The method of any one of claims 88-91, wherein the plurality of test conditions represents viral infections from 10 or more different viral species, 20 or more different viral species, or 30 or more viral species.

93. The method of any one of claims 88-92, wherein the plurality of test conditions represents bacterial infections from 10 or more different bacterial species, 20 or more different bacterial species, or 30 or more different bacterial species.

94. The method of any one of claims 88-93, wherein the set of time points consists of a single time point and the cross-reactivity of the gene signature is a mean of the AUROC value of each dataset in the plurality of datasets associated with a test condition that is other than the target condition.

95. The method of any one of claims 88-94, wherein
the set of time points is a plurality of time points,
the maximal AUROC value for each dataset in the plurality of datasets associated with the target condition is used to determine the performance of the gene signature, and
the maximal AUROC value for each dataset in the plurality of datasets associated with a test condition that is other than the target condition is used to determine the cross-reactivity of the gene signature.

96. The method of claim 88, wherein
each respective dataset in the plurality of datasets has, for each respective subject in the respective dataset, RNA-seq data for each gene in the first plurality of positive genes and each gene in the second plurality of positive genes, and
each dataset in the plurality of datasets comprises twenty or more subjects.

97. The method of claim 88, wherein the target condition is a first cancer type and each test condition in the plurality of test conditions is a different second cancer type.

98. The method of claim 88, wherein the target condition is a first degree of severity of a viral infection in the host species and a test condition in the plurality of test conditions is a second degree of severity of a viral infection in the host species.

99. The method of any one of claims 88-98, wherein the host species is human.

100. The method of any one of claims 88-99, wherein
the first plurality of positive genes consists of between three and thirty genes of the host species, and
the second plurality of negative genes consists of between three and thirty genes of the host species, other than the first plurality of positive genes.

101. The method of any one of claims 88-100, wherein
the first plurality of positive genes consists of between three and one hundred genes of the host species, and
the second plurality of negative genes consists of between three and one hundred genes of the host species, other than the first plurality of positive genes.

102. The method of any one of claims 88-101, wherein each dataset in the plurality of datasets comprises thirty or more subjects, forty or more subjects, 100 or more subjects, or between 5 and 1000 subjects.

103. A computer system for evaluating a gene signature associated with a target condition that can afflict a host species, wherein the gene signature comprises a first plurality of positive genes that are up-regulated when the test subject has the target condition and a second plurality of genes that are down-regulated when the test subject has the target condition, the computer system comprising:
one or more processors; and
memory addressable by the one or more processors, the memory storing at least one program for execution by the one or more processors, the at least one program comprising instructions for:
A) obtaining an indication of each gene in the first plurality of positive genes;
B) obtaining an indication of each gene in the second plurality of negative genes;
C) obtaining a plurality of datasets, wherein
each dataset in the plurality of datasets includes transcriptional data for each respective subject in a corresponding plurality of subjects and an indication of whether the respective subject has or does not have a respective test condition in a plurality of test conditions,
the plurality of datasets includes at least one dataset for each condition in the plurality of test conditions,
at least one test condition in the plurality of test conditions is the target condition,
D) for each respective dataset in a plurality of datasets, for each respective time point in a set of time points represented by the respective dataset:
for each respective subject in the respective dataset, determining a score for the respective subject at the respective time point by determining a difference between a geometric mean of abundance values for the first plurality of positive genes and a geometric mean of abundance values for the second plurality of positive genes indicated in the respective dataset,
determining an area under a receiver operator characteristic curve (AUROC) value for the respective dataset for the test condition using the respective score for each subject in the respective dataset at each respective timepoint;
E) evaluating a performance of the gene signature using the AUTROC value of each dataset in the plurality of datasets associated with the target condition; and
F) evaluating a cross-reactivity of the gene signature from the AUROC value of each dataset in the plurality of datasets associated with a test condition that is other than the target condition.

104. A non-transitory computer readable storage medium, wherein the non-transitory computer readable storage medium stores instructions, which when executed by a computer system, cause the computer system to perform a method for evaluating a gene signature associated with a target condition that can afflict a host species, wherein the gene signature comprises a first plurality of positive genes that are up-regulated when the test subject has the target condition and a second plurality of genes that are down-regulated when the test subject has the target condition, the method comprising:
A) obtaining an indication of each gene in the first plurality of positive genes;
B) obtaining an indication of each gene in the second plurality of negative genes;
C) obtaining a plurality of datasets, wherein
each dataset in the plurality of datasets includes transcriptional data for each respective subject in a corresponding plurality of subjects and an indication of whether the respective subject has or does not have a respective test condition in a plurality of test conditions,
the plurality of datasets includes at least one dataset for each condition in the plurality of test conditions,
at least one test condition in the plurality of test conditions is the target condition,
D) for each respective dataset in a plurality of datasets, for each respective time point in a set of time points represented by the respective dataset:
for each respective subject in the respective dataset, determining a score for the respective subject at the respective time point by determining a difference between a geometric mean of abundance values for the first plurality of positive genes and a geometric mean of abundance values for the second plurality of positive genes indicated in the respective dataset,
determining an area under a receiver operator characteristic curve (AUROC) value for the respective dataset for the test condition using the respective score for each subject in the respective dataset at each respective timepoint;
E) evaluating a performance of the gene signature using the AUROC value of each dataset in the plurality of datasets associated with the target condition; and
F) evaluating a cross-reactivity of the gene signature from the AUROC value of each dataset in the plurality of datasets associated with a test condition that is other than the target condition.

105. A method for determining whether a subject is infected with SARS-CoV-2, the method comprising:
obtaining a plurality of discrete attribute values, wherein each discrete attribute value in the plurality of discrete attribute values represents a transcript abundance of a respective gene in a plurality of genes in a biological sample from the subject, wherein the plurality of genes comprises three or more genes in the group consisting of PIF1, BANF1, ROCK2, DOCK5, SLK, TVP23B, GUDC1, ARAP2, SLC25A46, TCEAL3, and EHD3; and
inputting the plurality of discrete attribute values into a model comprising a plurality of parameters, wherein the model applies the plurality of parameters to the plurality of discrete attribute values to generate as output from the model an indication as to whether the subject is infected with SARS-CoV-2.

106. The method of claim 105, where the biological sample is a blood sample comprising plasmablast cells and T cells.

107. The method of claim 105 or 106, wherein the plurality of genes comprises PIF1 and EHD3.

108. The method of claim 106 or 106, wherein the plurality of genes comprises PIF1.

109. The method of claim 105, wherein the biological sample is a blood sample comprising at least plasmablast cells.

110. The method of any one of claims 105-109, wherein each discrete attribute value in in the plurality of discrete attribute values is determined by RNA-sequencing of the biological sample or by ATAC-sequencing of the biological sample.

111. The method of any one of claims 105-109, wherein the plurality of discrete attribute values is obtained by bulk transcriptome sequencing of nucleic acids in the biological sample.

112. The method of any one of claims 105-111, the method further comprising:
obtaining, in electronic form, a plurality of sequence reads from the biological sample, wherein the plurality of sequence reads comprises at least 10,000 RNA sequence reads; and
using the plurality of sequence reads to determine each discrete attribute value in the plurality of discrete attribute values.

113. The method of claim 112, wherein the using maps each respective sequence read in the plurality of sequence reads to a reference genome.

114. The method of claim 105, wherein the biological sample is blood, whole blood, or plasma.

115. The method of claim 105, wherein the biological sample comprises a plurality of mRNA molecules and the obtaining the plurality of sequence reads further comprises sequencing the plurality of mRNA molecules using RNA sequencing.

116. The method of claim 112, wherein the plurality of sequence reads comprises at least 100,000, at least 1×10⁶, or at least 1×10⁷ sequence reads.

117. The method of any one of claims 105-116, wherein the model is selected from the group consisting of: a logistic regression model, a neural network, a support vector machine, a Naive Bayes model, a nearest neighbor model, a boosted trees model, a random forest model, a decision tree, or a clustering model.

118. The method of any one of claims 105-117, wherein the plurality of parameters comprises 100 or more parameters, 1000 or more parameters, 10,000 or more parameters, 100,000 or more parameters, or 1×10⁶ or more parameters.

119. The method of any one of claims 105-118, wherein the indication as to whether the subject is infected with SARS-CoV-2 is a likelihood that the subject is infected with SARS-CoV-2.

120. The method of any one of claims 105-118, wherein the indication as to whether the subject is infected with SARS-CoV-2 is a binary indication as to whether or not the subject is infected with SARS-CoV-2.

121. The method claim 105, wherein the biological sample comprises serum, urine, cerebrospinal fluid, fecal, saliva, sweat, tears, pleural fluid, pericardial fluid, or peritoneal fluid from the subject.

122. The method of claim 105, wherein the biological sample consists of blood, whole blood, plasma, serum, urine, cerebrospinal fluid, fecal, saliva, sweat, tears, pleural fluid, pericardial fluid, or peritoneal fluid from the subject.

123. The method of any one of claims 105-122, wherein the plurality of genes comprises four, five, six, seven, eight, nine, or ten or more genes in the group consisting of PIF1, BANF1, ROCK2, DOCK5, SLK, TVP23B, GUDC1, ARAP2, SLC25A46, TCEAL3, and EHD3.

124. The method of any one of claims 105-122, wherein the plurality of genes consists of four, five, six, seven, eight, nine, or ten or more genes in the group consisting of PIF1, BANF1, ROCK2, DOCK5, SLK, TVP23B, GUDC1, ARAP2, SLC25A46, TCEAL3, and EHD3.

125. A computer system for determining whether a subject is infected with SARS-CoV-2, the computer system comprising:
one or more processors; and
memory addressable by the one or more processors, the memory storing at least one program for execution by the one or more processors, the at least one program comprising instructions for:
obtaining, in electronic form, a plurality of discrete attribute values, wherein each discrete attribute value in the plurality of discrete attribute values represents a transcript abundance of a respective gene in a plurality of genes in a biological sample from the subject, wherein the plurality of genes comprises three or more genes in the group consisting of PIF1, BANF1, ROCK2, DOCK5, SLK, TVP23B, GUDC1, ARAP2, SLC25A46, TCEAL3, and EHD3; and
inputting the plurality of discrete attribute values into a model comprising a plurality of parameters, wherein the model applies the plurality of parameters to the plurality of discrete attribute values to generate as output from the model an indication as to whether the subject is infected with SARS-CoV-2.

126. A non-transitory computer readable storage medium, wherein the non-transitory computer readable storage medium stores instructions, which when executed by a computer system, cause the computer system to perform a method for determining whether a subject is infected with SARS-CoV-2, the method comprising:
obtaining, in electronic form, a plurality of discrete attribute values, wherein each discrete attribute value in the plurality of discrete attribute values represents a transcript abundance of a respective gene in a plurality of genes in a biological sample from the subject, wherein the plurality of genes comprises three or more genes in the group consisting of PIF1, BANF1, ROCK2, DOCK5, SLK, TVP23B, GUDC1, ARAP2, SLC25A46, TCEAL3, and EHD3, and
inputting the plurality of discrete attribute values into a model comprising a plurality of parameters, wherein the model applies the plurality of parameters to the plurality of discrete attribute values to generate as output from the model an indication as to whether the subject is infected with SARS-CoV-2.

127. A method for determining whether a subject has a characteristic, the method comprising:
sequencing a plurality of mRNA molecules from a biological sample obtained from the subject, thereby obtaining a plurality of sequence reads of RNA from the subject;
aligning each respective sequence read in the plurality of sequence reads to a reference human transcriptome, thereby obtaining a corresponding plurality of aligned sequence reads;
using the corresponding plurality of aligned sequence reads to determine a corresponding transcript abundance in a plurality of transcript abundances, wherein each respective transcript abundance in the plurality of transcript abundances represents a transcript abundance of a corresponding gene in a plurality of genes;
inputting the plurality of transcript abundances into each respective neural network in a plurality of neural networks, wherein each respective neural network in the plurality of neural networks represents a different gene set in a plurality of gene sets, and wherein each respective neural network in the plurality of neural networks comprises:
(a) a corresponding plurality of input nodes, each respective input node in the corresponding plurality of input nodes for a different transcript abundance in the plurality of transcript abundance abundances, and
(b) a representation of the corresponding gene set in the form of (i) a corresponding plurality of hidden nodes, each hidden node representing a gene in the corresponding gene set, and (ii) a corresponding plurality of edges, wherein each edge in the corresponding plurality of edges interconnects an input node in the plurality of input nodes to a hidden node in the corresponding plurality of hidden nodes with a corresponding edge weight;
responsive to the inputting, obtaining a plurality of predictions, each prediction in the plurality of predictions from a neural network in the plurality of neural networks; and
responsive to inputting the plurality of predictions into an ensemble model obtaining, as output form the ensemble model a prediction of whether the subject has the characteristic.

128. The method of claim 127, wherein each corresponding plurality of hidden nodes consists of between three and ten hidden nodes.

129. The method of claim 127, wherein there are between three and twenty input nodes in the corresponding plurality of input nodes for each hidden node in the corresponding plurality of hidden nodes.

130. The method of any one of claims 127-129, wherein the characteristic is a disease state.

131. The method of any one of claims 127-129, wherein the characteristic is response to a drug.

132. The method of any one of claims 127-131, wherein each gene set in the plurality of gene sets represents a cellular function, a molecular pathway, or a mechanism for regulating gene expression.

133. The method of any one of claims 127-129, wherein the characteristic is an indication as to whether or not the subject is experiencing kidney transplant rejection.

134. The method of any one of claims 127-133, wherein the plurality of gene sets consists of between 100 genes sets and 15,000 gene sets and each gene set in the plurality of gene sets comprises three or more genes.

135. The method of any one of claims 127-133, wherein the plurality of gene sets consists of between 100 genes sets and 15,000 gene sets and each gene set in the plurality of gene sets consists of between three genes and 100 genes.

136. The method of any one of claims 127-135, the method further comprising log-normalizing the corresponding plurality of aligned sequence reads.

137. The method of any one of claims 127-136, wherein for each respective neural network in the plurality of neural networks, each respective edge in the corresponding plurality of edges has a nonzero weight when it couples a first gene, associated with an input node in the corresponding plurality of input nodes, to a second gene associated with a corresponding hidden node, in the corresponding plurality of hidden nodes, that are known from a prior knowledge to interact with each other in accordance with a cellular function, a molecular pathway, or a mechanism for regulating gene expression associated with the corresponding gene set.

138. The method of any one of claims 127-137, wherein the plurality of sequence reads comprises at least 10,000, at least 100,000, at least 1×10⁶, or at least 1×10⁷ sequence reads.

139. The method of any one of claims 127-138, wherein the biological sample comprises blood, serum, urine, cerebrospinal fluid, fecal, saliva, sweat, tears, pleural fluid, pericardial fluid, or peritoneal fluid from the subject.

140. The method of any one of claims 127-139, wherein the biological sample consists of blood, serum, urine, cerebrospinal fluid, fecal, saliva, sweat, tears, pleural fluid, pericardial fluid, or peritoneal fluid from the subject.

141. The method of any one of claims 127-139, wherein the biological sample is a tissue sample from the subject.

142. A computer system for determining whether a subject has a characteristic, the computer system comprising:
one or more processors; and
memory addressable by the one or more processors, the memory storing at least one program for execution by the one or more processors, the at least one program comprising instructions for:
aligning each respective sequence read in a plurality of sequence reads, wherein the plurality of sequence reads represent a plurality of mRNA molecules in a biological sample obtained from the subject, to a reference human transcriptome, thereby obtaining a corresponding plurality of aligned sequence reads;
using the corresponding plurality of aligned sequence reads to determine a corresponding transcript abundance in a plurality of transcript abundances, wherein each respective transcript abundance in the plurality of transcript abundances represents a transcript abundance of a corresponding gene in a plurality of genes;
inputting the plurality of transcript abundances into each respective neural network in a plurality of neural networks, wherein each respective neural network in the plurality of neural networks represents a different gene set in a plurality of gene sets, and wherein each respective neural network in the plurality of neural networks comprises:
(a) a corresponding plurality of input nodes, each respective input node in the corresponding plurality of input nodes for a different transcript abundance in the plurality of transcript abundance abundances, and
(b) a representation of the corresponding gene set in the form of (i) a corresponding plurality of hidden nodes, each hidden node representing a gene in the corresponding gene set, and (ii) a corresponding plurality of edges, wherein each edge in the corresponding plurality of edges interconnects an input node in the plurality of input nodes to a hidden node in the corresponding plurality of hidden nodes with a corresponding edge weight;
responsive to the inputting, obtaining a plurality of predictions, each prediction in the plurality of predictions from a neural network in the plurality of neural networks; and
responsive to inputting the plurality of predictions into an ensemble model obtaining, as output form the ensemble model a prediction of whether the subject has the characteristic.

143. A non-transitory computer readable storage medium, wherein the non-transitory computer readable storage medium stores instructions, which when executed by a computer system, cause the computer system to perform a method for determining whether a subject has a characteristic, the method comprising:
aligning each respective sequence read in a plurality of sequence reads, wherein the plurality of sequence reads represent a plurality of mRNA molecules in a biological sample obtained from the subject, to a reference human transcriptome, thereby obtaining a corresponding plurality of aligned sequence reads;
using the corresponding plurality of aligned sequence reads to determine a corresponding transcript abundance in a plurality of transcript abundances, wherein each respective transcript abundance in the plurality of transcript abundances represents a transcript abundance of a corresponding gene in a plurality of genes;
inputting the plurality of transcript abundances into each respective neural network in a plurality of neural networks, wherein each respective neural network in the plurality of neural networks represents a different gene set in a plurality of gene sets, and wherein each respective neural network in the plurality of neural networks comprises:
(a) a corresponding plurality of input nodes, each respective input node in the corresponding plurality of input nodes for a different transcript abundance in the plurality of transcript abundance abundances, and
(b) a representation of the corresponding gene set in the form of (i) a corresponding plurality of hidden nodes, each hidden node representing a gene in the corresponding gene set, and (ii) a corresponding plurality of edges, wherein each edge in the corresponding plurality of edges interconnects an input node in the plurality of input nodes to a hidden node in the corresponding plurality of hidden nodes with a corresponding edge weight;
responsive to the inputting, obtaining a plurality of predictions, each prediction in the plurality of predictions from a neural network in the plurality of neural networks; and
responsive to inputting the plurality of predictions into an ensemble model obtaining, as output form the ensemble model a prediction of whether the subject has the characteristic.

144. A method for determining one or more transcription factors that regulate a first gene in a cell type, the method comprising:
A) obtaining a single nucleus multi-omics dataset, in electronic form, comprising:
(i) a respective ATAC fragment count for each ATAC peak in a corresponding plurality of ATAC peaks, for each respective cell in a plurality of cells, and
(ii) a respective discrete attribute value for each gene transcript in a corresponding plurality of gene transcripts, for each respective cell in the plurality of cells, wherein the plurality of cells is from a biological sample from a subject;
B) obtaining a plurality of transcription factor binding sites, wherein each respective transcription factor binding site in the plurality of transcription factor binding sites is associated with (i) a gene in a plurality of genes and (ii) a transcription factor in a plurality of transcription factors;
C) for each respective cell represented in the plurality of cells, for each respective transcription factor binding site in the plurality of transcription factor binding sites, using the respective ATAC fragment count for each corresponding ATAC peak from the respective cell in the single nucleus multi-omics dataset within a threshold distance of the respective transcription factor binding site to determine a respective binary openness assignment for the respective transcription factor binding site for the respective cell represented in the plurality of cells;
D) for each respective cell represented in the plurality of cells, for each respective gene in the plurality of genes, wherein the plurality of genes includes the first gene, forming a respective regressor of form:
$$z = f ( {y}_{ij} \cdot {x}_{i} )$$
wherein,
z is the respective discrete attribute value of the respective gene for the respective cell in the single nucleus multi-omics dataset,
xᵢ is the respective discrete attribute value of the ith transcription factor associated with the respective gene for the respective cell in the single nucleus multi-omics dataset, and
yᵢⱼ is the binary openness of the jth transcription factor binding site of the ith transcription factor in the respective cell,
ƒ is a linear model, and
i and j are positive integers,
thereby forming a plurality of regressors; and
E) regressing the plurality of regressors against the single nucleus multi-omics dataset, thereby identifying one or more transcription factors in the plurality of transcription factors that regulate the first gene.

145. The method of claim 144, wherein a first transcription factor binding site in the plurality of transcription factor binding sites is associated with a first transcription factor in the plurality of transcription factors when the first transcription factor binding site is within a window around a start site of the first transcription factor.

146. The method of claim 145, wherein the window is +/−50 kilobases, +/−100 kilobases, +/−150 kilobases, or +/−200 kilobases around a start site of the first transcription factor.

147. The method of any one of claims 144-146, wherein the threshold distance is a value between 25 bases and 1000 bases.

148. The method of any one of claims 144-146, wherein the threshold distance is 400 bases.

149. The method of any one of claims 144-148, wherein the plurality of cells comprises a plurality of cell types and the method further comprises using the plurality of regressors to identify one or more transcription factors in the plurality of transcription factors that regulate the first gene in a first cell type in the plurality of cell types.

150. The method of claim 149, wherein the plurality of cell types comprises 2, 3, 4, 5, 6, 7, 8, 9, or 10 different cell types.

151. The method of any one of claims 144-150, wherein the plurality of cells comprises 50 or more cells, 100 or more cells or 1000 or more cells.

152. The method of any one of claims 144-151, wherein
each corresponding plurality of gene transcripts represents 50 or more genes, and
each corresponding plurality of ATAC peaks comprises 50 or more peaks.

153. The method of any one of claims 144-152, wherein the plurality of genes comprises 2, 3, 4, 5, 6, 7, 8, 9, or 10 genes.

154. The method of any one of claims 144-152, wherein the plurality of genes comprises 10 or more, 20 or more, or 100 or more genes.

155. The method of any one of claims 144-152, wherein the plurality of genes consists of between 2 and 15000 genes.

156. The method of any one of claims 144-155, wherein the plurality of regressors comprises between twenty and one thousand regressors.

157. The method of any one of claims 144-155, wherein the plurality of regressors comprises 100 or more regressors.

158. The method of any one of claims 144-157, wherein the biological sample comprises blood, serum, urine, cerebrospinal fluid, fecal, saliva, sweat, tears, pleural fluid, pericardial fluid, or peritoneal fluid from the subject.

159. A computer system for determining one or more transcription factors that regulate a first gene in a cell type, the computer system comprising:
one or more processors; and
memory addressable by the one or more processors, the memory storing at least one program for execution by the one or more processors, the at least one program comprising instructions for:
A) obtaining a single nucleus multi-omics dataset, in electronic form, comprising:
(i) a respective ATAC fragment count for each ATAC peak in a corresponding plurality of ATAC peaks, for each respective cell in a plurality of cells, and
(ii) a respective discrete attribute value for each gene transcript in a corresponding plurality of gene transcripts, for each respective cell in the plurality of cells, wherein the plurality of cells is from a biological sample from a subject;
B) obtaining a plurality of transcription factor binding sites, wherein each respective transcription factor binding site in the plurality of transcription factor binding sites is associated with (i) a gene in a plurality of genes and (ii) a transcription factor in a plurality of transcription factors;
C) for each respective cell represented in the plurality of cells, for each respective transcription factor binding site in the plurality of transcription factor binding sites, using the respective ATAC fragment count for each corresponding ATAC peak from the respective cell in the single nucleus multi-omics dataset within a threshold distance of the respective transcription factor binding site to determine a respective binary openness assignment for the respective transcription factor binding site for the respective cell represented in the plurality of cells;
D) for each respective cell represented in the plurality of cells, for each respective gene in the plurality of genes, wherein the plurality of genes includes the first gene, forming a respective regressor of form:
$$z = f ( {y}_{ij} \cdot {x}_{i} )$$
wherein,
z is the respective discrete attribute value of the respective gene for the respective cell in the single nucleus multi-omics dataset,
xᵢ is the respective discrete attribute value of the ith transcription factor associated with the respective gene for the respective cell in the single nucleus multi-omics dataset, and
yᵢⱼ is the binary openness of the jth transcription factor binding site of the ith transcription factor in the respective cell,
ƒ is a linear model, and
i and j are positive integers,
thereby forming a plurality of regressors; and
E) regressing the plurality of regressors against the single nucleus multi-omics dataset, thereby identifying one or more transcription factors in the plurality of transcription factors that regulate the first gene.

160. A non-transitory computer readable storage medium, wherein the non-transitory computer readable storage medium stores instructions, which when executed by a computer system, cause the computer system to perform a method for determining one or more transcription factors that regulate a first gene in a cell type, the method comprising:
A) obtaining a single nucleus multi-omics dataset, in electronic form, comprising:
(i) a respective ATAC fragment count for each ATAC peak in a corresponding plurality of ATAC peaks, for each respective cell in a plurality of cells, and
(ii) a respective discrete attribute value for each gene transcript in a corresponding plurality of gene transcripts, for each respective cell in the plurality of cells, wherein the plurality of cells is from a biological sample from a subject;
B) obtaining a plurality of transcription factor binding sites, wherein each respective transcription factor binding site in the plurality of transcription factor binding sites is associated with (i) a gene in a plurality of genes and (ii) a transcription factor in a plurality of transcription factors;
C) for each respective cell represented in the plurality of cells, for each respective transcription factor binding site in the plurality of transcription factor binding sites, using the respective ATAC fragment count for each corresponding ATAC peak from the respective cell in the single nucleus multi-omics dataset within a threshold distance of the respective transcription factor binding site to determine a respective binary openness assignment for the respective transcription factor binding site for the respective cell represented in the plurality of cells;
D) for each respective cell represented in the plurality of cells, for each respective gene in the plurality of genes, wherein the plurality of genes includes the first gene, forming a respective regressor of form:
$$z = f ( {y}_{ij} \cdot {x}_{i} )$$
wherein,
z is the respective discrete attribute value of the respective gene for the respective cell in the single nucleus multi-omics dataset,
xᵢ is the respective discrete attribute value of the ith transcription factor associated with the respective gene for the respective cell in the single nucleus multi-omics dataset, and
yᵢⱼ is the binary openness of the jth transcription factor binding site of the ith transcription factor in the respective cell,
ƒ is a linear model, and
i and j are positive integers,
thereby forming a plurality of regressors; and
E) regressing the plurality of regressors against the single nucleus multi-omics dataset, thereby identifying one or more transcription factors in the plurality of transcription factors that regulate the first gene.
